(19) United States
(12) Patent Application Publication
(10) Pub. No.: US 2026/0311813 A1
(43) Pub. Date: Oct. 8, 2026

(54) METHODS FOR GENE TRAINING

(71) Applicant: MENDAGENE LLC, Johnston, IA (US)

(72) Inventor: Edward Jason STANEK, Johnston, IA (US)

(21) Appl. No.: 19/425,652

(22) Filed: Dec. 18, 2025

Related U.S. Application Data
(63) Continuation-in-part of application No. 19/244,001, filed on Jun. 20, 2025.
(60) Provisional application No. 63/731,933, filed on Jun. 21, 2024.

(51) Int. Cl. G16B 30/20; G16B 25/10

(52) U.S. Cl. CPC G16B 30/20; G16B 25/10

(57) ABSTRACT

Specific proteins catalyze specific reactions, transport specific molecules, and form specific cellular structures and changing their presence and relative abundance can be applied, for example to improve human health, animal health, crop health, carbon capture, agricultural productivity and protection, and biologic activity. However, precisely effectuating phenotypic changes and outcomes is expensive and error prone; and precisely changing protein expression in an organism has been challenging due to limited ability to control experimental factors, limitations of measurement, the exponential number of ways to affect gene expression of a protein, and limited precision and accuracy in predicting changes to expression when modifying transcription factors, promoters, and GC content. This disclosure introduces methods of gene training to help enable more direct, expedient and successful product development pipelines to digitally adjust or aim expression level of one or more proteins, such as for improving crops, carbon capture, biologics, research and development, and human health.

### CROSS-REFERENCE TO RELATED APPLICATIONS

This application is a continuation-In-Part of U.S. patent application Ser. No. 19/244,001, filed on Jun. 20, 2025, which claims the benefit of U.S. Provisional Patent Application No. 63/731,933, filed on Jun. 21, 2024, both of which are incorporated by reference herein in their entirety.

### REFERENCE TO SEQUENCE LISTING

This application contains a sequence listing, which is submitted electronically as a XML formatted sequence listing with a file name “M4GT-2025-06-08.xml”, creation date Jun. 9, 2025, and having a size of 301,409 bytes. The sequence listing submitted electronically is part of the specification and is herein incorporated by reference in its entirety.

### FIELD OF THE INVENTION

This invention relates generally to methods for sequence design by gene training and their applications. Gene training is the precise aiming, focusing, or adjusting of gene expression to a desired extent, degree, and/or resolution. More particularly, the present disclosure relates to methods for crop improvement, carbon capture, livestock improvement, biologics improvement, biological discovery research and development, and human health improvement.

### BACKGROUND OF THE INVENTION

The demand for food is expected to increase by 50% by 2050 while there is increasing scarcity of limited resources. Crop performance improvement is an expensive process done with multiple stages and gates. Tools for crop improvement include breeding pollinations, transferring genes from one organism to another, altering gene promoters, introducing random mutations (for example through irradiation, amino acid sequence perturbations, GC content, or aligning with genome-wide codon frequencies), and introducing so-called nonsense mutations or deletions.

US healthcare spending is close to five trillion dollars annually, currently two fifths of which is paid for by the federal government. Pharmaceutical consumption alone has reached $600 billion annually in the United States. The average cost of developing a new pharmaceutical is reported to be around $1 billion. Gene therapies are reported to be a promising approach to medicine. However, only one in five gene therapies receive approval.

### SUMMARY OF THE INVENTION

The present application discloses hereby invented new methods for designing a polynucleotide sequence capable of training expression of a target protein or polypeptide to a desired relative expression level. In some embodiments, an example comprises obtaining two sequences representing RNA that encode the protein or polypeptide, wherein one of the RNAs is capable of expressing the target protein or polypeptide at a greater level than the other; selecting differences between the two sequences representing RNA, wherein a third sequence representing potential RNA encoding the protein is generated that differs from one of the two RNA sequences by the selected differences. In some methods, the obtaining step is performed by obtaining a sequence representation of RNA with an expression level greater than the desired expression level and obtaining another sequence representation of RNA with an expression level less than the desired expression level. In some methods, the selecting step is performed by choosing differences between sequences representing codons wherein each codon represents a possible encoding of an amino acid within the protein sequence translatable from RNA. In some methods, the method includes obtaining a sequence representation of RNA from an in silico reverse translation method, such as CDSFold. In some methods, a nucleotide analogue might be used in place of a native nucleotide in an RNA molecule, wherein a nucleotide analogue may be selected from: pseudouridine, inosine, I-methyl-pseudouridine, and 5-methyl-cytidine, 1-methoxy-pseudouridine, and pseudo-isocytidine.

In some methods, the method further comprises designing a specification for treating an organism with polynucleotide molecules, for example by indicating a configuration that the delivery mechanism and polynucleotide deliverables must have. In some methods, a design might comprise formulating a gene therapy for medical use. In some methods, the formulation of the design for medical use might comprise combining the gene therapy molecule with one or more of: a buffer, a lubricant, a binder, a flavorant, a coating, and an adjuvant. In some methods, the specification requires the gene therapy molecule comprises or is encapsulated via carrier, with the carrier optionally selected from: a virus, an adeno-associated virus, a viroid, a virion, a capsid, a micelle, a vesicle, a lipid nanoparticle, a protein nanoparticle, a DNA structure such as a 3DNA or DNA origami, an RNA Structure, or analogue thereof. In some methods, the carrier comprising or of the designed therapeutic is decorated with transporter-specific proteins. In some methods, the carrier or the designed therapeutic is decorated with autologous HLA proteins to mitigate immunogenicity. In some methods, the design specification includes an antisense oligonucleotide (ASO). In some methods, the ASO is designed to target a specific gene variant RNA or RNA of an allele. In some methods, the ASO is specific to one or more target RNAs transcribed from the gene variant. In some methods, the ASO is specific to one or more target RNAs transcribed from the gene variant, but a proper prefix of the sequence of the ASO is not sufficiently specific to ensure nontargeting of an RNA transcribed from a different variant of the same or different. In still further methods, the ASO is designed to specifically to target one or more RNAs transcribed from the gene variant, but a suffix of the sequence of the ASO is not sufficiently specific to ensure nontargeting of an RNA transcribed from a different gene or gene variant. In some methods, the ASO targets one or more RNAs transcribed from the gene variant, but neither a suffix nor a prefix of the sequence of the ASO is sufficiently specific to be an ASO that targets RNA transcribed from the gene variant, but not another variant of the gene or a different gene. In some methods, the design specification of a treatment includes a plurality of ASOs.

In some methods, an example comprises specifying requirements for editing DNA in a genome to transcribe to the improved RNA molecule for agricultural use. In some methods, a method to arrive at a specification of DNA editing comprises: determining the difference between the sequence of a desired RNA sequence and the sequence of a putative RNA sequence encoding of the desired protein; mapping the putative RNA sequence to the subsequence of the DNA sequence that is capable of being transcribed into the sequence of the putative RNA encoding the protein; and using the mapping to create a specification for editing the DNA to be capable of transcribing desired RNA with designed expression level, comprising a sequence of edits identifying polynucleotides of the sequence of DNA to change to create a new DNA sequence, a DNA molecule thereof capable of transcribing in cell the desired RNA molecule. In some methods, a difference is determined by aligning the ith nucleotide of the desired sequence to the ith nucleotide of the existing sequence. In some methods, the mapping of RNA to DNA is a set of DNA transcription intervals obtainable from: transcription tracing, transformation tracking, or aligning the sequence of RNA to the sequence of DNA. In some methods, the specification of editing DNA may comprise one or more site-specific or site-directed methods from: DNA editing, recombinase mediated cassette exchange, transformation, and other genetic engineering techniques. In some methods, a DNA editing method may be selected from: genome editing, gene editing, prime editing, twin prime editing, and base editing.

In some methods, the method comprises designing specifications to training expression of a plurality of proteins. In some methods, the specification of a plurality of proteins may originate from recommendations from: a systems biology method, solver, simulation, or experimentation. In some methods, the expression level of the plurality of proteins is specified from a user interface.

In some methods, the design specifications are created via a semi-automated, or partially-automated system, or fully-automated system. In some methods, the system may specify to design in parallel. In some methods, a method of breeding may be specified from: trait integration, backcrossing, continuous breeding, speed breeding, forward breeding, and trait stacking.

The present application discloses hereby invented new computer-aided methods for designing a sequence for a polypeptide-inducing polynucleotide that induces a specified polypeptide whose translation will help train expression of one or more proteins comprising the polypeptide to a desired relative expression level, the method comprising: generating a sequence of a polypeptide-inducing polynucleotide that induces the specified protein with the approximate expression design level in one step. In some methods, the method comprises specifying a sequence representing a potential nucleic acid for putatively delivering in cell an mRNA composed of or transcribed from the polypeptide-inducing polynucleotide, in order to deliver a therapeutic protein or therapeutic ASO. In some examples, the protein of the mRNA may be produced in cell via transcription of the polypeptide-inducing polynucleotide. In some examples, the expression design level of protein inducible from the polypeptide-inducing polynucleotide is or is correlated with a difference percentage used as the desired relative expression level. In some methods, an example comprises specifying one or more constraints on the set of nucleotides considered to be acceptable at one or more positions within the generated sequence. In some methods, an example comprises specifying one or more untranslated regions to use for generating a sequence using one or more constraints. In some methods, the method allows specification of a set of nucleotides considered to be acceptable at one or more positions in the design of the sequence and optionally includes nucleotide variants considered to be natural variation.

The present application discloses hereby invented new computer-aided methods for designing a sequence for a polypeptide-inducing polynucleotide that will help train expression of an induced polypeptide to a desired relative expression level, the method comprising: obtaining a first sequence representing a first mRNA encoding the polypeptide with half-life longer than or equal to the half-life of a minimum MFE mRNA that encodes the polypeptide; and generating a second mRNA sequence, the mRNA encoding the polypeptide, wherein the second mRNA design has a half-life estimate longer than the half-life estimate of the first mRNA design, wherein in cell the polypeptide-inducing polynucleotide is or is capable of being transcribed and/or spliced to the second mRNA. In some methods, a method further comprises specifying a sequence for producing in cell the second mRNA identical to or transcribed and/or spliced from the polypeptide-inducing polynucleotide, wherein the encoded protein would be produced in greater quantity by the second mRNA than if the coding region of the polypeptide-inducing polynucleotide were instead the coding region of the first mRNA. In some methods the second mRNA may be produced via transcription of the polypeptide-inducing polynucleotide of the designed sequence. In some methods, the second mRNA may be produced via splicing of the polypeptide-inducing polynucleotide or of a transcript of the polypeptide-inducing polynucleotide of the designed sequence. In some methods, the method comprises identifying a first sequence of first polynucleotide capable of being transcribed in a cell; generating a second sequence of a second polynucleotide capable of being transcribed (and optionally spliced) to the mRNA of the second sequence; and using a technique to facilitate cellular entry for therapeutic use for human health or in agricultural crop improvement to apply a genetic engineering method to edit the genome of the cell to transcribe the second polynucleotide in place of the first polynucleotide.

The present application discloses hereby invented a methods for designing a sequence for a polypeptide-inducing polynucleotide that can help train expression of a polypeptide to a desired relative expression level comprising: obtaining two sequences of (potential) RNA that encode the polypeptide, wherein one of the RNA sequences has a greater expression design level than the other; and selecting differences between the sequences representing the two RNAs, wherein a polypeptide-inducing polynucleotide sequence that induces the polypeptide is generated from a third RNA sequence design, wherein the third RNA sequence differs from one of the first two RNA sequences by the selected differences, wherein the third RNA sequence design has an expression design level intermediate of the design levels of the RNAs of the first two sequences. In some methods, examples comprises specifying to produce in cell an mRNA comprising the RNA of the third sequence, wherein the mRNA is or is transcribed and/or spliced from the polypeptide-inducing polynucleotide. In some methods, the mRNA is to be produced via transcription of the polypeptide-inducing polynucleotide of the sequence design, or is to be spliced from the polypeptide-inducing polynucleotide of the sequence design, and/or is a spliced from a transcript of the polypeptide-inducing polynucleotide of the sequence design. In some methods, either or both of the sequences of the obtaining step represent existing RNA, wherein the expression level of each existing RNA differs from the desired expression level. In some methods, either or both of the sequences from existing RNA are considered wildtype. In some methods, either or both of the sequences of the obtaining step are considered a reference or consensus sequence. In some methods, the sequence of RNA of the obtaining step has a minimum or minimal length-adjusted MFE, optionally subject to a set of constraints. In some methods, the sequence of RNA of the obtaining step has a maximum or maximal length-adjusted MFE, optionally subject to a set of constraints. In some methods, the sequence of RNA of the obtaining step is a sequence of mRNA. In some methods, the sequence of mRNA of the obtaining step has a half-life estimate longer than the half-life estimate of an mRNA with a maximum or maximal length-adjusted MFE, optionally subject to a set of constraints or has a half-life estimate shorter than the half-life estimate of an mRNA with a minimal length-adjusted MFE, optionally subject to a set of constraints. In some methods, the selecting step comprises selecting a difference set comprising indices and/or pairs of indices, wherein an indice may represent codon position in its codon sequence, wherein the codon sequence represents the respective coding region. In some methods, the selecting step comprises selecting a difference set wherein each pair of indices in the difference identifies the indices of nucleotides in the respective sequence. In some methods, the amino acid sequence of the protein comprising the polypeptide encoded by the first RNA is the same as the amino acid sequence of the protein comprising the polypeptide encoded by the second RNA. In some methods, one or more untranslated regions of the first RNA are the same as the respective untranslated region(s) of the second RNA.

In some methods, a series of sequences may be obtained, wherein each sequence in the series corresponds to a selected difference set, wherein every difference set that precedes another difference set in the series is a subset thereof, or wherein every difference set that succeeds another difference set in the series is a subset thereof. In some methods, a series of sets of sequences is obtained, wherein each sequence in a set in the series was subjected to a test, wherein the difference set implying each sequence is the group tested, wherein each difference set implying each sequence in a set of the series was chosen using a method of group testing. In some methods, a series of sequences is obtained, wherein selection of each of the differences in the series comprises a search via a divide and conquer approach, such as a divide and conquer approach comprising a binary search and/or a level lowering approach.

The present application also discloses hereby invented new computer-aided method for designing sequences of mRNA that help train expression level of a second protein to the expression level of another protein, comprising: identifying the sequence of a first mRNA encoding the first protein, wherein the first mRNA has a first half-life; determining a half-life estimate of the first mRNA; and generating a second mRNA encoding the second protein, wherein the second mRNA has a half-life estimate approximately the same as the half-life estimate of the first mRNA. In some methods, the second protein may be functionally analogous to the first protein. In some methods, the second protein and first protein may have the same enzymatic classification and/or Enzyme Commission number. In some methods, the second mRNA is analogous to the first mRNA and/or mobility analogous to the first mRNA. In some methods, the second mRNA and first mRNA have the same predicted subcellular localizations. In some methods, the RNAs are CDS RNAs and the expression design level is selected from or correlated with a negative length-adjusted MFE, a percentage. In some examples, the RNAs are mRNAs and the expression design level is selected from or correlated with a half-life, a negative length-adjusted MFE, a percentage. In some methods, the method further comprises testing of generated sequences for the satisfaction of specified required properties, wherein each sequence in the obtained series satisfies the required properties. In some methods, invariance of predicted untranslated region secondary structure is a required property. In some methods, if two sequences differ at one element according to an identity alignment and exactly one of the two sequences satisfies the specified required properties, then the element is retained in all subsequent difference sets that imply sequences in the series. In some methods, the element is a codon. In some methods the element is a nucleotide. In some methods, the element may in certain cases be either.

Some methods, further comprise specifying either or both of a 5′ untranslated region that flanks the coding regions of the CDS RNAs on the 5′ side and a 3′ untranslated region that flanks the coding regions of the CDS RNAs on the 3′ side. In some methods, a method further comprises designing a specification for treatment of an organism with the polypeptide-inducing polynucleotide molecule. In some methods, the treatment design comprises specifying for formulation the polypeptide-inducing polynucleotide molecule for medical use. In some methods, if the specification for polynucleotide is transcription capable, the polynucleotide may include a promoter to control the context of expression. In some methods, the specified promoter may be tetracycline-dependent to allow for controlled transcription. In some methods, the formulation is to be specified by combining the polypeptide-inducing polynucleotide molecule with one or more of: a buffer, a lubricant, a binder, a flavorant, a coating, and an adjuvant. In some methods, the specification of treatment design further comprises a delivery mechanism of the polypeptide-inducing polynucleotide molecule, optionally wherein the capsule/carrier is selected from: 3DNA, a DNA structure, a virus, an adeno-associated virus, a viroid, a virion, a capsid, a micelle, a vesicle, a lipid nanoparticle, a protein nanoparticle, and an RNA Structure. In some methods, the delivery mechanism of the specification is to be decorated with transporter-specific proteins specific to the target tissue and/or cell type. In some methods, the delivery mechanism of the specification is to be decorated with autologous human leukocyte antigen (HLA) proteins specific to the intended treatment recipient. In some methods, the design specification further comprises allowing substitution of at least one nucleotide analogue for a native nucleotide in the polypeptide-inducing polynucleotide molecule. In some examples, the nucleotide analog might be selected from pseudouridine, inosine, I-methyl-pseudouridine, and 5-methyl-cytidine, 1-methoxy-pseudouridine, and pseudo-isocytidine. In some examples, the treatment design specification further comprises designing a specification for treating an organism with an antisense oligonucleotide (ASO).

The present application also discloses hereby invented new methods of generating a sequence for a polypeptide-inducing polynucleotide such that a produced mRNA would be fortified against an ASO, wherein the difference subset is selected so that the generated sequence is fortified against the ASO. The present application also discloses hereby invented new methods comprising of generating a sequence for a polypeptide-inducing polynucleotide such that a produced mRNA is fortified against the ASO, wherein the constraints are selected so that the generated sequence representing the polypeptide-inducing polynucleotide is fortified against the ASO. The present application also relates to a method wherein a sequence representing a pathogenicity-specific supplemental and derived RNA is fortified against an ASO.

The present application also discloses hereby invented a new computer-aided method of designing a specification for a sequence for an antisense oligonucleotide that can be used to aim or help aim expression of a polypeptide to some desired expression levels comprising obtaining a sequence representing a polynucleotide such that its pairing-sensitive reverse complement is a signature of one or more sequences representing a transcript or spliced version (or portion thereof) of a target gene variant with respect to a reference set; wherein the reference set comprises one or more transcriptomes and optionally one or more genomes. In some examples, the antisense oligonucleotide of the design specification is a pareto-specific ASO or a MS-ASO. In some methods, the sequence is pairing-sensitive to a transcript against a reference set comprising a reference transcriptome and/or a reference genome. In some methods, the sequence is pairing-sensitive to a class of transcripts with a specific mutation against a reference set comprising a reference transcriptome and/or a reference genome or sequences thereof.

The present application also discloses hereby invented new computer-aided methods of checking for off-target potential of an ASO comprising: obtaining sequence data; and computing the loci and/or variants to which the ASO may bind via computational homology.

The present application also discloses hereby invented new methods of identifying signatures comprising use of a suffix automaton or an equivalent thereof. In some methods, the method of identifying signatures comprises use of matrix multiplication or an equivalent thereof. In some methods, the method comprises memoization of matrix multiplications corresponding to substrings of the target transcript. In some methods, an example comprises specification of properties for a treatment for an organism by the ASO molecule. In some methods, an example involves designing a specification for a polynucleotide comprising the ASO or a polynucleotide from which the ASO molecule is transcribed. In some methods, an example may require specification of a polynucleotide that comprises a promoter to control the context of expression, wherein the promoter is optionally dependent on a small molecule, for example optionally, tetracycline-dependent, to allow for controlled transcription.

In some methods, the design further comprises specification for carrying the deliverables. In some methods, the specification of carrier/capsule is selected from: 3DNA, a DNA structure, a virus, an adeno-associated virus, a viroid, a virion, a capsid, a micelle, a vesicle, a lipid nanoparticle, a protein nanoparticle, and an RNA Structure, or analogue thereof. In some methods, the capsule/carrier specification is to include decoration by transporter-specific proteins specific to the target tissue and/or cell type. In some examples, the capsule/carrier specification is to include decoration by autologous human leukocyte antigen (HLA) proteins specific to the intended treatment recipient to mitigate immunogenicity. In some methods, the design further comprises substituting at least one nucleotide analog for a native nucleotide in the ASO molecule, which optionally is selected from pseudouridine, inosine, I-methyl-pseudouridine, and 5-methyl-cytidine, 1-methoxy-pseudouridine, and pseudo-isocytidine.

The present application also discloses hereby invented new methods of specification of treatment for an organism including one or more pathogenicity-specific supplemental or pathogenicity-specific suppressive and optionally additional polynucleotides.

The present application also discloses hereby invented new computer-aided methods of screening an organism for suitability to treat the organism with a supplemental, suppressive, or multi-modal modality for a pathogenic genetic condition comprising: detecting a sequence containing a pathogenic signature that uniquely identifies a locus and allele in the organism, wherein the pathogenic sequence indicates the pathogenic genetic condition; and affirming suitability of the modality if the modality is among a list of acceptable modalities according to the phenotype of the pathogenic genetic condition, wherein determination of suitability is further refined for a modality that comprises a suppressive. In some methods the list of acceptable modalities for a pathogenic genetic condition with Mendelian phenotype of autosomal recessive or X-linked recessive is a pathogenicity-specific supplemental. In some methods, if the pathogenic genetic condition of the specific pathogenic variant has the autosomal dominant phenotype, then the list of acceptable modalities consists of: (1) a suppressive for the detected pathogenic sequence when the cause comprises a transcript of the pathogenic sequence that is actively disruptive; (2) a set of suppressives wherein the set consists of a suppressive for one out of each set of benign co-expressed variants that remain expressed in duplicate (due to ploidy) in order that balanced co-expression ratio be restored, when the cause comprises unbalanced co-expression; and (3) a set of co-expressed supplementals necessary to restore expression level sufficiency while maintaining balanced co-expression, when the cause comprises haploinsufficiency. In some methods, if the pathogenic genetic condition of the specific pathogenic variant has the X-linked dominant phenotype, then the list of acceptable modalities consists of: (1) a suppressive for the detected pathogenic sequence when the cause comprises a transcript of the pathogenic sequence that is actively disruptive; (2) a set of suppressives wherein the set consists of a suppressive for one out of each set of benign co-expressed variants that remain expressed in duplicate (due to ploidy) in order that balanced co-expression ratio be restored, when the cause comprises unbalanced co-expression; and (3) a set of co-expressed supplementals necessary to restore expression level sufficiency while maintaining balanced co-expression, when the cause comprises haploinsufficiency. In some methods, the supplementals may be fortified against included suppressive(s) as necessary. In some methods, the list of acceptable modalities for a pathogenic genetic condition with Y-linked, cytoplasmic inheritance, or incomplete-dominance, and the pathogenic condition is caused solely by a missing functional transcript is a pathogenicity-specific supplemental. In some methods, the list of acceptable modalities for a pathogenic genetic condition is suppressive for Y-linked, cytoplasmic inheritance, or incomplete-dominance, pathogenic conditions when the cause comprises a transcript of the pathogenic sequence that is actively disruptive. In some methods, the list of acceptable modalities for a pathogenic genetic condition with Y-linked, cytoplasmic inheritance, or incomplete-dominance, and the pathogenic condition is caused both by a missing functional transcript and by an actively disruptive transcript is multimodal, comprising a suppressive for the detected pathogenic sequence and a pathogenicity-specific supplemental. In some methods, if there does not exist a needed pathogenicity-specific suppressive, then a non-specific suppressive must not be delivered without a pathogenicity-specific supplemental and must use a mechanism of co-delivery. In some methods, if the modality is multimodal, the supplementals may be fortified against included suppressive(s) as necessary. In further embodiments, wherein the pathogenic sequence is detected in RNA or DNA or sequences thereof of the organism. In some methods, the reverse complement of the pathogenic sequence is detected in DNA or sequences thereof of the organism.

The present application also discloses hereby invented new methods of designing specification of a nucleic acid therapy for personalized medical treatment to reduce, mute, delay, slow, prevent, or treat an undesired genetic condition according to the pathogenicity-specific phenotype comprising one or more a pathogenicity-specific supplemental or suppressive for the genetic condition, wherein if the therapy includes both a suppressive and a supplemental then each included supplemental is fortified against each included suppressive. In some methods, if a supplemental is included in the therapy and the supplemental encodes a wildtype protein, then the subcellular localization of the therapy is predicted to equal or approximates the subcellular localization of a wildtype mRNA encoding the wildtype protein. In some examples, the nucleic acid therapy further comprises a delivery mechanism. In some examples, the delivery mechanism may support transcription of one or more polynucleotides. In some examples, the delivery mechanism may support protein decorations. In some examples, a protein decoration may include a protein to help direct the deliverables(s) to specific tissue(s) and/or cell(s). In some examples, a protein decoration may include one or more autologous human leukocyte antigen (HLA) proteins to help reduce immunogenicity. In some methods, transcription may be dependent upon one or more promoters, wherein a promoter may be optionally tetracycline-dependent. In some methods, the delivery mechanism may support design of a stoichiometric delivery ratio for balancing co-expression. In some methods, the delivery mechanism may be selected from a 3DNA, a DNA structure, a virus, an adeno-associated virus, a viroid, a virion, a capsid, a micelle, a vesicle, a lipid nanoparticle, a protein nanoparticle, and an RNA Structure. In some examples, the nucleic acid therapy is for an autosomal recessive disorder phenotype. In some examples, the nucleic acid therapy comprises designs for a pathogenicity-specific supplemental of hemoglobin subunit beta (HBB) for an autosomal recessive Sickle Cell genetic disorder or designs for a pathogenicity-specific supplemental of hexosaminidase A (HEXA) for an autosomal recessive Tay-Sachs genetic disorder. In some examples, the specification is for design of a nucleic acid therapy is for an X-linked recessive disorder phenotype or an autosomal dominant disorder phenotype.

In some examples, the specification of nucleic acid therapy comprises sequences of a pathogenicity-specific suppressive for a pathogenic transthyretin (TTR) RNA for an autosomal dominant Familial Amyloid Polyneuropathy and/or Amyloidogenic Transthyretin Amyloidosis disorder. In some examples, the specification of nucleic acid therapy comprises sequences of a pathogenicity-specific suppressive for a pathogenic tumor protein p53 (TP53) RNA together with a sequence for a supplemental for a non-pathogenic TP53 for an autosomal dominant Li-Fraumeni syndrome disorder. In some examples, the nucleic acid therapy comprising sequences for a co-expression balance restoring suppressive or multimodal involving PKD1 and/or PKD2 for an autosomal dominant polycystic kidney disease (ADPKD) disorder.

In some hereby invented and disclosed new methods are computer-aided methods of specification of personalized medical treatment comprising: screening the genetic and/or transcriptional data of an organism for one or more pathogenic sequences; identifying of one or more prognosticated genetic conditions in the organism associated with the pathogenic sequences; and obtaining one or more mending treatment sequence designs that are pathogenicity-specific for the pathogenic sequences contained therein. In some methods, the screening step further comprises: detection of one or more locus signatures; and either detection of a known pathogenic signature; or detection of a sequence (or subsequence thereof) in a relevant and appropriate pathogenic reference set but not in a relevant and appropriate non-pathogenic reference set. In some methods, the obtaining step may further comprises signaling via electronic communication to initiate retrieving, ordering, and/or producing the sequence design for a mending treatment for the detected pathogenic sequence of the screening step. In some methods, the obtaining step may further comprises: retrieving information about or describing relevant pathogenic variant(s), relevant loci(us), relevant pathogenic genetic condition(s), relevant pathogenicity-specific polynucleotides(s), and/or relevant, available, and/or producible mending treatments from a database, dictionary, index, catalogue, or other information store using a pathogenic signature and/or locus signature. In some methods, the method further comprises specification of a quality control step after the obtaining step wherein sequence designs and/or their metadata are to be checked for appropriate modality for the pathogenic genetic condition(s).

The present application discloses hereby invented new methods for identifying a likely splicing from one polynucleotide to a second polynucleotide that utilizes dynamic programming together with longest common prefix queries between suffixes of the sequences of the two polynucleotides.

The present application discloses hereby invented new methods for designing a specification for editing DNA in a genome to be transcribed and/or spliced into an mRNA for agricultural use. In some methods the specification step comprises: determining the difference between the desired RNA sequence and an existing RNA sequence encoding of the polypeptide; mapping the difference to the sequence of DNA that is transcribed into the existing RNA encoding the polypeptide; and identifying the (poly)nucleotides to substitute into the DNA molecule to create a substituted DNA molecule that transcribes and/or splices the desired RNA sequence. In some methods, the difference is determined by an order-preserving alignment that includes all the positions of the existing RNA sequence or an order-preserving alignment that includes all the positions of the desired RNA sequence. In some methods, the mapping is a set of sequence intervals that describe transcription obtained from data. In additional examples, the specification of substitution of (poly)nucleotides identified comprises specification of one or more site-specific or site-directed methods from: DNA editing, recombinase mediated cassette exchange, and transformation. In some examples, the specification of DNA editing method is selected from: genome editing, gene editing, prime editing, twin prime editing, and base editing. In some examples, the specification of the type of DNA editing process is selected from: gene modification and gene replacement.

In some embodiments, the method further comprises computer-aided design of a plurality of sequences of polynucleotides to help train expression of a plurality of proteins comprising polypeptides. In some examples, the plurality of polypeptides may be selected using: a systems biology method, solver, simulation, or experimentation. In some examples, the expression design level or direction of change of one or more of the polynucleotide sequences may be a recommendation from: a systems biology method, solver, simulation, or experimentation. In some methods, the expression design level of one or more of the sequences for polynucleotide designs is specified from a user interface. In some methods, the design of the sequences with specified expression level design(s) is computer-automated. In some methods, the design of the sequences may be carried out in parallel.

The present application also discloses hereby invented new computer-aided methods for identifying needed genome editing to assess a novel polypeptide sequence, the methods comprising: identifying the sequence of a first potential polynucleotide capable of being transcribed (and optionally spliced) into a first mRNA encoding a first protein, wherein the first mRNA has a first half-life estimate; generating the sequence of a second potential polynucleotide capable of being transcribed (and optionally spliced) into a second mRNA encoding the novel polypeptide, wherein the second mRNA has the same half-life estimate as the first mRNA;

wherein the sequence of the first protein does not comprise the novel polypeptide; and wherein editing of a genome by genetic engineering such that it transcribes the second polynucleotide in the place of the first polynucleotide is an enabler for assessment of the novel polypeptide sequence.

The present application discloses hereby invented new computer-aided methods for designing specifications to identify needs for editing a genome to assess a novel expression level of a polypeptide, a methods comprising: identifying the sequence of a first polynucleotide capable of being transcribed (and optionally spliced) into a first mRNA encoding the polypeptide, wherein the first mRNA has a first half-life estimate; generating the sequence of a second polynucleotide capable of being transcribed (and optionally spliced) into a second mRNA encoding the polypeptide, wherein the second mRNA has a novel half-life estimate, wherein the first half-life estimate differs from the novel half-life estimate; wherein the sequence of the second mRNA differs from the sequence of the sequence of the first mRNA according to an identity alignment; and wherein editing of a genome by genetic engineering to facilitate transcription of the second polynucleotide in the place of the first polynucleotide would help enable assessment of the novel expression level.

The present application also discloses hereby invented new computer-aided methods for assessing phenotypes, the method comprising: a conventional method of signal analysis that includes a term for each polypeptide of interest, wherein the term captures (a) the effect of the (difference in) expression level of the polypeptide; and/or (b) the effect of the identity of the polypeptide.

The present application also discloses hereby invented new computer-aided methods for determining a set of a plurality of polypeptides whose expression levels increases are together necessary to enhance a biological pathway, the method comprising: performing group testing on subsets of the set of genes implicated by a signal analysis, wherein a test comprises an increased expression level of all the genes in the subset relative to the expression levels used to conduct the signal analysis.

The present application also discloses hereby invented new computer-aided methods for designing specifications for editing a genome to produce a novel expression level of one or more proteins comprising a polypeptide, the methods comprising: identifying the sequence of a first polynucleotide capable of being transcribed (and optionally spliced) into a first mRNA encoding the polypeptide, wherein the first mRNA has a first half-life estimate; generating the sequence of a second polynucleotide capable of being transcribed (and optionally spliced) into a second mRNA encoding the polypeptide, wherein the second mRNA has a novel half-life estimate, wherein the first half-life estimate differs from the novel half-life estimate; wherein the sequence of the second mRNA differs from the sequence of the first mRNA according to an identity alignment; and wherein editing of a genome by genetic engineering to facilitate transcription of the second polynucleotide in the place of the first polynucleotide would help enable the edited genome to produce the polypeptide at the novel expression level in cell.

The present application discloses hereby invented new computer-aided methods for designing specifications to identify needs for editing a genome to produce a novel polypeptide variant at a desired expression level, the methods comprising: identifying the sequence of a first polynucleotide capable of being transcribed (and optionally spliced) into a first mRNA encoding a first protein, wherein the first mRNA has a first half-life estimate; generating the sequence of a second polynucleotide capable of being transcribed (and optionally spliced) into a second mRNA encoding the novel polypeptide variant, wherein the second mRNA has a desired half-life estimate; wherein the sequence of the first protein does not comprise the novel polypeptide variant; and wherein editing of a genome by genetic engineering to transcribe the second polynucleotide in the place of the first polynucleotide enables an edited genome produce a novel polypeptide variant at the desired expression level in cell.

The present application also discloses hereby invented new methods for designing specifications for introducing new genetic variation into a germplasm pool comprising the methods and techniques described elsewhere herein.

In a further aspect, the present application also discloses hereby invented new methods for designing a sequence for a polynucleotide to aim or help aim expression of a polypeptide in a cell, the methods comprising: obtaining a sequence of a potential first polynucleotide, wherein the first polynucleotide is capable of being transcribed into first mRNA encoding the polypeptide, wherein the first mRNA has a first half-life; generating a sequence of a potential second polynucleotide, wherein the second polynucleotide is capable of being transcribed into a second mRNA encoding the polypeptide, wherein the second mRNA has a second half-life estimate that differs from the first half-life estimate; wherein the second mRNA differs from the first mRNA according to an order-preserving alignment with minimum difference; and wherein if the second half-life estimate is longer than the first half-life estimate, then on average the encoded polypeptide would be produced in greater quantity by the second mRNA molecule in cell than would be produced by the first mRNA molecule in cell or if the second half-life is shorter than the first half-life, then on average the encoded polypeptide would be produced in lesser quantity by the second mRNA molecule in cell than would be produced by the first mRNA molecule in cell. In some examples, the second half-life estimate is longer than the first half-life estimate. In some examples, the second half-life estimate is shorter than the first half-life estimate. In some examples, the protein produced from the second mRNA has an amino acid sequence that is the same as the protein produced from the first mRNA. In some examples, the first nucleotide sequence is a wildtype sequence.

The present application discloses hereby invented new methods for designing a sequence for a polynucleotide to aim or help aim expression of one or more proteins comprising a polypeptide in a cell, the methods comprising: obtaining a sequence of a first polynucleotide capable of being transcribed into a first mRNA encoding the polypeptide, wherein the first mRNA has a first half-life estimate; obtaining a sequence of a second polynucleotide capable of being transcribed into a second mRNA encoding the polypeptide, wherein the second mRNA has a second half-life estimate, and wherein the sequence of the second mRNA differs from the sequence of the first mRNA according to an order-preserving alignment with minimum size difference; generating a sequence of a third polynucleotide capable of being transcribed into a third mRNA encoding the target peptide, wherein the third mRNA has a third half-life estimate, wherein the sequence of the third mRNA differs from the sequences of the first mRNA and second mRNA according to order-preserving alignments with minimum size differences, wherein the third half-life estimate is intermediate to the first half-life and the second half-life; and wherein if the third half-life is longer than the first half-life, the one or more proteins comprising the polypeptide will on average be produced in greater quantity by the third mRNA molecule in cell than would be from the first mRNA molecule in cell or if the third half-life estimate is shorter than the first half-life estimate, then the one or more proteins comprising the polypeptide will be produced in lesser quantity by the third mRNA molecule in cell than would be from the first mRNA molecule in cell. In some examples, the second mRNA has a maximal or minimum MFE for mRNAs encoding the polypeptide. In some examples, the first mRNA has a maximal or minimum MFE estimate for mRNAs encoding the polypeptide. In some examples, a MFE estimate of the third mRNA is greater than a MFE estimate of the second mRNA. In some examples, a MFE estimate of the third mRNA is less than a MFE estimate of the second mRNA. In some examples, a protein producible from the third mRNA has an amino acid sequence that is the same as a protein producible from the first mRNA. In some examples, the first polynucleotide sequence is a wildtype sequence.

The present application discloses hereby invented new methods for designing a polynucleotide sequence whose implementation in cell would help aim expression of a target polypeptide, the methods comprising: identifying a first sequence of a first polynucleotide capable of being transcribed into a first mRNA encoding the target peptide, wherein the first mRNA has a first half-life estimate; generating a second sequence of a second polynucleotide capable of being transcribed into a second mRNA encoding the target peptide, wherein the second mRNA has a second half-life estimate; wherein the second sequence varies from the first mRNA by one or more nucleotides and wherein, as a result of the nucleotide variations, the second half-life estimate differs from the first half-life estimate; and wherein if producing in a cell the second polynucletide, the cell produces the target polypeptide from the second mRNA, wherein if the second half-life estimate is longer than the first half-life estimate, then in aggregate the one or more proteins comprising the target polypeptide will be produced in greater quantity than if producing in a cell the first polynucleotide, or if the second half-life estimate is shorter than the first half-life estimate, then in aggregate the one or more proteins comprising the target polypeptide will be produced in lesser quantity than if producing in a cell the first polynucleotide. In some examples, the methods further comprise identifying needs for gene editing for agriculture or needs for in vivo therapy comprising entry into the cell the second polynucleotide, wherein said edits may be accomplished by genetic engineering or said entry may be accomplished by endocytosis. In some examples, the first and second mRNA each have a minimum free energy (MFE), and wherein the second mRNA has a MFE estimate that differs from the MFE estimate of the first mRNA. In some examples, the MFE estimate of the second mRNA is greater than the MFE estimate of the first mRNA. In some examples, the MFE estimate of the second mRNA is less than the MFE estimate of the first mRNA. In some examples, the second half-life estimate is longer than the first half-life estimate, while in some examples the second half-life estimate is shorter than the first half-life estimate. In some examples, the target peptide produced from the second mRNA has an amino acid sequence that is identical to the target peptide produced from the first mRNA. In additional examples, the first polynucleotide sequence is a wildtype sequence.

The present application discloses hereby invented new methods for designing a sequence to aim or help aim expression of one or more proteins comprising a target polypeptide in a cell, the methods comprising: identifying a first sequence of a first polynucleotide capable of being transcribed into a first mRNA encoding the target polypeptide, wherein the first mRNA has a first minimum free energy (MFE) estimate; identifying a second sequence of a second polynucleotide capable of being transcribed into a second mRNA encoding the target polypeptide, wherein the second mRNA has a second MFE estimate, wherein the sequence of the second mRNA varies from the sequence of the first mRNA by a plurality of nucleotides, wherein the second MFE estimate differs from the first MFE estimate; generating a sequence of a third polynucleotide capable of being transcribed into a third mRNA encoding the target polypeptide, wherein the third mRNA has a third MFE estimate, wherein the sequence of the third mRNA varies from the sequences of the first and second mRNAs by a plurality of nucleotides, wherein the third MFE estimate is intermediate to the first MFE estimate and the second MFE estimate; wherein if producing in a cell the third polynucleotide, the cell would produce the target polypeptide from the third mRNA, wherein if the third MFE estimate has value lesser than the first MFE estimate, then the proteins comprising the polypeptide produced from the target polypeptide would be produced in greater quantity than would be from the first mRNA or if the third MFE estimate has value greater than the first MFE estimate, then the target polypeptide would be produced in lesser quantity than would from the first mRNA. In some embodiments the method further comprises the step of specifying gene editing in agriculture or to entry into the cell in gene therapy such that the third nucleotide sequence will reside in cell, wherein said editing is accomplished by genetic engineering and said entry if via endocytosis. In some embodiments, the second MFE estimate is a maximum or minimum MFE estimate for mRNAs encoding the target polypeptide. In further embodiments, the MFE estimate from the third mRNA sequence is less than the MFE estimate from the first mRNA sequence. In still further embodiments the MFE estimate for the third mRNA sequence is greater than the MFE estimate of the first mRNA sequence. In some examples, a protein produced from the third mRNA has an amino acid sequence that is identical to the protein produced from the first mRNA. In some examples, the sequence of the first nucleotide sequence is a wildtype sequence.

The present application discloses hereby invented new methods of sequence design of a polynucleotide for improving the expression of a target polypeptide to improve a performance capability of a genome of an organism for agriculture or carbon capture comprising: identifying a sequence of a first polynucleotide that induces the target polypeptide and is capable of being transcribed to a first RNA whose sequence has first expression design level, wherein the target polypeptide is encoded by the first mRNA; generating a sequence of a second polynucleotide that induces the target polypeptide and is capable of being transcribed to a second RNA whose sequence has second expression design level, wherein the target polypeptide is encoded by the second mRNA, wherein the second expression design level differs from the first expression design level, wherein the sequence of the second polynucleotide differs from the sequence of the first polynucleotide according to an order-preserving alignment that aligns all the positions of the first polynucleotide or aligns all the positions of the second polynucleotide; wherein a genome edited to comprise the second polynucleotide in place of the first polynucleotide would express collectively a greater quantity in protein molecules comprising the target polypeptide than in the scenario the genome comprised the first polynucleotide when the second mRNA sequence has greater expression design level than that of the sequence of the first mRNA, and wherein the second polynucleotide expresses collectively the proteins comprising the target polypeptide in lesser quantity than the first polynucleotide when the second mRNA sequence has lesser expression design level than that of the first mRNA sequence, whereby the difference in protein expression may improve, alone or requiring the adjustment of expression levels of other mRNAs in tandem, a performance capability of an organism with the changed genome for agriculture or carbon capture. In some embodiments, if the second expression design level is greater than the first expression design level, then the change may improve the performance capability alone or require the increase of expression levels of other mRNAs in tandem. In other embodiments, if the second expression design level is less than the first expression design level, then the change would improve the performance capability. In some methods, the second expression design level is used to aim or help aim expression level. In some methods, the second expression design level is aimed to a specified quantity. In some methods, the specified quantity is determined from the sequence of an identified third polynucleotide that induces the target polypeptide and is capable of being transcribed to a third RNA whose sequence has third expression design level. In some methods, the second expression design level is equal to the third expression design level. In some examples, the second expression design level is greater than the third expression design level. In some examples, the second expression design level is less than the third expression design level. In yet further examples, the quantity described by each expression design level is or is correlated with a negative length-adjusted minimum free energy (MFE) estimate. In some examples, the quantity described by each expression design level is or is correlated with a half-life. In some examples, the quantity described by each expression design level is a percentage.

In some methods, the quantity described by each expression design level is or is correlated with a protein production capacity per mRNA molecule. In some methods, the quantity described by each expression design level is equal to transcription initiation efficiency times protein production capacity per mRNA molecule. In some methods, if a change comprises gene editing of the first polynucleotide to the second polynucleotide or a gene replacement of the first polynucleotide by the second polynucleotide, then the modification improves the performance capability. In some methods, the change comprises a gene therapy via cellular entry for human health or editing of the second polynucleotide in a cell or editing for agriculture, crop improvement, and carbon capture. In some examples, a protein produced from the second mRNA has an amino acid sequence may be identical to the amino acid sequence of a protein produced from the first mRNA. In other examples, the first mRNA may be wildtype and the second mRNA differs from wildtype.

In some examples, the protein comprising the target polypeptide is an enzyme of a hormone producing pathway, the performance capability is increased development, and the organism is of the type harvested for agriculture. In some examples, the second mRNA has lower half-life than the first mRNA, the enzyme comprises a maize GA20ox3, the stalk circumference has increased development, and the organism is a maize plant. In some examples, the second mRNA sequence has higher expression design level than the sequence of the first mRNA, the enzyme comprises a maize GA20ox3, one or more florescence features of have increased development in the absence of a damaging wind event, and the organism is a maize plant. In some examples, the florescence features comprise increased kernel row number or increased yield.

In some examples, the protein comprising the target polypeptide is an insect toxin, the performance capability is resistance to insect pests, and the organism is of the type harvested for agriculture. In some examples, the second mRNA has longer half-life than the first mRNA, the insect toxin comprises a maize Cry3Bb1, the performance capability is increased trait durability of resistance is to Coleopteran, and the organism is a maize plant. In some examples, the protein comprising the target polypeptide is an enzyme of a chemical defense pathway, the performance capability is resistance to bacteria, fungus, and/or pests, and the organism is of the type harvested for agriculture. In some examples, the chemical defense pathway produces DIMBOA glucosides and the organism is a maize plant. In some examples, the second mRNA whose sequence has higher expression design level than the sequence of the first mRNA and the target polypeptide comprises a maize DIMBOA UDP-glucosyltransferase.

In some examples, the protein comprising the target polypeptide is an enzyme designed for herbicide tolerance, the performance capability is tolerance to a herbicide. In some examples, the sequence of the second mRNA has higher expression design level than the sequence of the first mRNA, the protein comprising the target polypeptide is a maize acetolactate synthase that was redesigned towards herbicide tolerance, the herbicide comprises a sulfonylurea and/or triazolopyrimidine herbicide, and the organism is a maize plant. In some examples, the sequence of the second mRNA has lower half-life estimate than that of the sequence of the first mRNA, the protein comprising the target polypeptide is considered a fruit size regulator, and the performance capability is increased fruit size. In some examples, the protein comprising the target polypeptide is considered to be the fruit size regulator CLAVATA3 and the organism is a tomato plant or an eggplant plant.

In some examples, the sequence of the second mRNA has greater expression design level than that of the sequence of the first mRNA, the target polypeptide is a root growth factor inducible transcription factor, the performance capability is increased root growth, and the organism is a crop valued for root growth.

### BRIEF DESCRIPTION OF THE FIGURES

FIG. 1: Agriculture improvement business process diagram.

FIG. 2: Health improvement business process diagram.

FIG. 3: Example User Interface for Exploratory Analysis.

FIG. 4A-4C: SEQ ID NO: 5 shown as sequence of codons, a CDS sequence trained to a 45.1% expression design level encoding of the Zm00001eb366090_P001 variant of a GA20ox3 protein of Zea mays.

FIG. 5A-5D: SEQ ID NO: 12 shown as a sequence of codons, a CDS sequence trained to a 60% expression design level encoding of a Cry3Bb1 protein variant from U.S. Pat. No. 7,705,216.

FIG. 6A-6D: SEQ ID NO: 13 shown as a sequence of codons, a CDS sequence trained to a 50% expression design level encoding of a Cry3Bb1 protein variant from U.S. Pat. No. 7,705,216.

FIG. 7A-7D: SEQ ID NO: 14 shown as a sequence of codons, a CDS sequence trained to a 40% expression design level encoding of a Cry3Bb1 protein variant from U.S. Pat. No. 7,705,216.

FIG. 8A-8B: SEQ ID NO: 55 shown as a sequence of codons with CDS trained to a 50.3% expression design level encoding of a Zm00001eb284010_P001 variant of a ZAG1 protein of Zea mays.

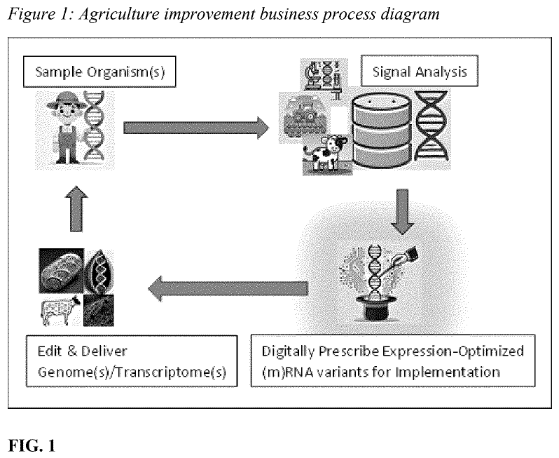

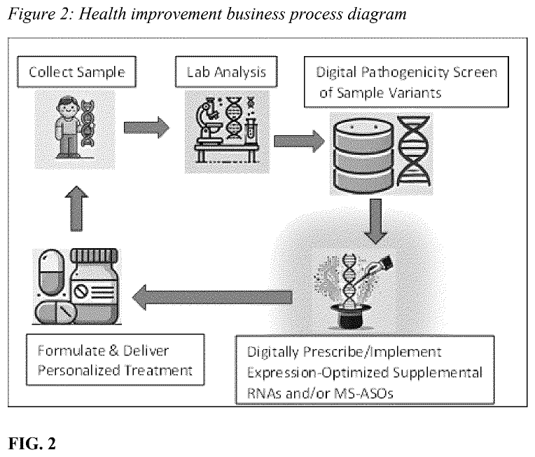

### BRIEF DESCRIPTION OF THE SEQUENCES

SEQ ID NO: 1 provides the amino acid sequence of the Zm00001eb366090_P001 variant of a GA20ox3 protein of Zea mays.

SEQ ID NO: 2 provides a design for a nucleic acid CDS sequence trained to a 100% expression design level encoding of the Zm00001eb366090_P001 variant of a GA20ox3 protein of Zea mays.

SEQ ID NO: 3 provides a design for a nucleic acid CDS sequence trained to a 0% expression design level encoding of the Zm00001eb366090_P001 variant of a GA20ox3 protein of Zea mays.

SEQ ID NO: 4 provides the nucleic acid sequence of the Zm00001eb366090_T001 CDS encoding of the Zm00001eb366090_P001 variant of a GA20ox3 protein of Zea mays.

SEQ ID NO: 5 provides a design for a nucleic acid CDS sequence trained to a 45.1% expression design level encoding of the Zm00001eb366090_P001 variant of a GA20ox3 protein of Zea mays.

SEQ ID NO: 6 provides a design for a nucleic acid CDS sequence trained to a 44.9% expression design level encoding of the Zm00001eb366090_P001 variant of a GA20ox3 protein of Zea mays.

SEQ ID NO: 7 provides the polynucleotide sequence of the coding strand of the DNA region transcribed then spliced to the Zm00001eb366090_T001 mRNA encoding of the Zm00001eb366090_P001 variant of a GA20ox3 protein of Zea mays.

SEQ ID NO: 8 provides a design for a polynucleotide sequence to help identify what needs DNA editing with CDS trained to a 44.9% expression design level encoding of the Zm00001eb366090_P001 variant of a GA20ox3 protein of Zea mays.

SEQ ID NO: 9 provides the amino acid sequence of a variant of Cry3Bb1 protein from U.S. Pat. No. 7,705,216 SEQ ID NO: 10 provides a design for a nucleic acid CDS sequence trained to a 44.9% expression design level encoding of the Zm00001eb366090_P001 variant of a GA20ox3 protein of Zea mays.

SEQ ID NO: 11 provides a design for a nucleic acid CDS sequence trained to a 0% expression design level encoding of a Cry3Bb1 protein variant from U.S. Pat. No. 7,705,216.

SEQ ID NO: 12 provides a design for a nucleic acid CDS sequence trained to a 60% expression design level encoding of a Cry3Bb1 protein variant from U.S. Pat. No. 7,705,216.

SEQ ID NO: 13 provides a design for a nucleic acid CDS sequence trained to a 50% expression design level encoding of a Cry3Bb1 protein variant from U.S. Pat. No. 7,705,216.

SEQ ID NO: 14 provides a design for a nucleic acid CDS sequence trained to a 40% expression design level encoding of a Cry3Bb1 protein variant from U.S. Pat. No. 7,705,216.

SEQ ID NO: 15 provides the polynucleotide sequence of the coding strand of the DNA region transcribed then spliced to the Zm00001eb379110_T001 CDS encoding of the Zm00001eb379110_P001 variant of a Transcription factor MYB39 protein of Zea mays.

SEQ ID NO: 16 provides a design for a polynucleotide sequence to help identify what needs DNA editing with CDS trained to a 53% expression design level with putative encoding of the Zm00001eb379110_P001 variant of a Transcription factor MYB39 protein of Zea mays.

SEQ ID NO: 17 provides the polynucleotide sequence of the coding strand of the DNA region transcribed then spliced to the Zm00001eb379120_T001 transcript with CDS encoding of the Zm00001eb379120_P001 variant of an uncharacterized protein of Zea mays.

SEQ ID NO: 18 provides a design for a polynucleotide sequence to help identify what needs DNA editing with CDS trained to a 65% expression design level encoding of a Zm00001eb379120_P001 variant of an uncharacterized protein of Zea mays.

SEQ ID NO: 19 provides the polynucleotide sequence of the coding strand of the DNA region transcribed then spliced to the Zm00001eb379130_T001 transcript with CDS encoding of the Zm00001eb379130_P001 variant of an Haloacid dehalogenase-like hydrolase domain-containing protein Sgpp of Zea mays.

SEQ ID NO: 20 provides a design for a polynucleotide sequence to help identify what needs DNA editing with CDS trained to a 56% expression design level encoding of a Zm00001eb379130_P001 variant of an Haloacid dehalogenase-like hydrolase domain-containing protein Sgpp of Zea mays.

SEQ ID NO: 21 provides the polynucleotide sequence of the coding strand of the DNA region transcribed then spliced to the Zm00001eb379140_T001 transcript with CDS encoding of the Zm00001eb379140_P001 variant of a different Haloacid dehalogenase-like hydrolase domain-containing protein Sgpp of Zea mays.

SEQ ID NO: 22 provides a design for a polynucleotide sequence to help identify what needs DNA editing with CDS trained to a 57% expression design level encoding of a Zm00001eb379140_P001 variant of the preceding Haloacid dehalogenase-like hydrolase domain-containing protein Sgpp of Zea mays.

SEQ ID NO: 23 provides the polynucleotide sequence of the coding strand of the DNA region transcribed then spliced to the Zm00001eb064870_T001 transcript with CDS encoding of the Zm00001eb064870_P001 variant of a Phosphoglycolate phosphatase protein of Zea mays.

SEQ ID NO: 24 provides a design for a polynucleotide sequence to help identify what needs DNA editing with CDS trained to a 11% expression design level with putative encoding of a Zm00001eb064870_P001 variant of the Phosphoglycolate phosphatase protein of Zea mays.

SEQ ID NO: 25 provides the polynucleotide sequence of the coding strand of the DNA region transcribed then spliced to the Zm00001eb299920_T001 transcript with CDS encoding of the Zm00001eb299920_P001 variant of a Glyoxylate reductase protein of Zea mays.

SEQ ID NO: 26 provides a design for a polynucleotide sequence to help identify what needs DNA editing with CDS trained to a 74% expression design level with putative encoding of a Zm00001eb299920_P001 variant of the Glyoxylate reductase protein of Zea mays.

SEQ ID NO: 27 provides the amino acid sequence of the RITF1 protein of Arabidopsis thaliana.

SEQ ID NO: 28 provides the amino acid sequence of the Zm00001eb234360_P001 variant of a putative homolog of Arabidopsis thaliana RITF1 protein from Zea mays.

SEQ ID NO: 29 provides the nucleic acid sequence of the Zm00001eb234360 T001 CDS encoding of the Zm00001eb234360_P001 variant of a putative homolog of Arabidopsis thaliana RITF1 protein from Zea mays.

SEQ ID NO: 30 provides a design for a CDS sequence trained to a 40.2% expression design level encoding of a Zm00001eb234360_P001 variant of a putative homolog of Arabidopsis thaliana RITF1 protein of Zea mays.

SEQ ID NO: 31 provides a design for a polynucleotide sequence to help identify what needs DNA editing with CDS trained to a 40.2% expression design level with putative encoding of a Zm00001eb234360_P001 variant of a putative homolog of Arabidopsis thaliana RITF1 protein of Zea mays.

SEQ ID NO: 32 provides a design for a polynucleotide sequence to help identify what needs DNA editing in a gene replacement with intron sequences excluded with CDS trained to a 40.2% expression design level encoding of a Zm00001eb234360_P001 variant of a putative homolog of Arabidopsis thaliana RITF1 protein of Zea mays.

SEQ ID NO: 33 provides the amino acid sequence of the Zm00001eb165590_P001 variant of a BX8 DIMBOA UDP-glucosyltransferase protein of Zea mays.

SEQ ID NO: 34 provides the nucleic acid sequence of the Zm00001eb165590_T001 CDS encoding of the Zm00001eb165590_P001 variant of a BX8 DIMBOA UDP-glucosyltransferase protein (also annotated as a glycosyltransferase) from Zea mays.

SEQ ID NO: 35 provides a design for a nucleic acid CDS sequence trained to a 65.0% expression design level encoding of a Zm00001eb165590_P001 variant of a BX8 DIMBOA UDP-glucosyltransferase protein of Zea mays.

SEQ ID NO: 36 provides a design for a polynucleotide sequence to help identify what needs DNA editing with CDS trained to a 65.0% expression design level with putative encoding of a Zm00001eb165590_P001 variant of a BX8 DIMBOA UDP-glucosyltransferase protein of Zea mays.

SEQ ID NO: 37 provides the amino acid sequence of the Zm00001eb241810_P001 variant of an acetolactate synthase 1 protein of Zea mays.

SEQ ID NO: 38 provides a design for a variant Zm00001eb241810_P001 sequence with hra-like mutations towards herbicide tolerance, wherein Zm00001eb241810_P001 is an acetolactate synthase 1 protein of Zea mays.

SEQ ID NO: 39 provides the nucleic acid sequence of the Zm00001eb241810 T001 CDS encoding of the Zm00001eb241810_P001 variant of a herbicide-susceptible acetolactate synthase (ALS1) protein from Zea mays.

SEQ ID NO: 40 provides a design for a nucleic acid sequence with CDS trained to a 45.2% expression design level encoding of a synthetic design of a variant of Zm00001eb241810_P001 with hra-like mutations towards herbicide tolerance, wherein Zm00001eb241810_P001 is an acetolactate synthase 1 protein of Zea mays.

SEQ ID NO: 41 provides a design for a polynucleotide sequence to help identify what needs DNA editing with CDS trained to a 45.2% expression design level with putative encoding of a design for a variant Zm00001eb241810_P001 sequence with hra-like mutations, wherein Zm00001eb241810_P001 is an acetolactate synthase 1 protein of Zea mays.

SEQ ID NO: 42 provides the amino acid sequence of the Zm00001eb180890_P001 variant of an herbicide-susceptible acetolactate synthase 2 protein of Zea mays.

SEQ ID NO: 43 provides a design for a variant Zm00001eb180890_P001 sequence with hra-like mutations towards herbicide tolerance, wherein Zm00001eb180890_P001 is an acetolactate synthase 2 protein of Zea mays.

SEQ ID NO: 44 provides the nucleic acid sequence of the Zm00001eb180890 T001 CDS encoding of the Zm00001eb180890_P001 variant of a different herbicide-susceptible acetolactate synthase (ALS2) protein from Zea mays.

SEQ ID NO: 45 provides a design for a nucleic acid sequence with CDS trained to a 45.2% expression design level encoding of a design for a Zm00001eb180890_P001 variant with hra-like mutations, wherein Zm00001eb180890_P001 is an acetolactate synthase 2 protein of Zea mays.

SEQ ID NO: 46 provides a design for a polynucleotide sequence to help identify what needs DNA editing with CDS trained to a 45.2% expression design level with putative encoding of a design of a Zm00001eb180890_P001 variant with hra-like mutations, wherein Zm00001eb180890_P001 is an acetolactate synthase 2 protein of Zea mays.

SEQ ID NO: 47 provides the amino acid sequence of the Zm00001eb145600_P002 variant of an GA20ox4 protein of Zea mays.

SEQ ID NO: 48 provides the nucleic acid sequence of the Zm00001eb366090_T001 CDS encoding of the Zm00001eb366090_P001 variant of a GA20ox3 protein from Zea mays.

SEQ ID NO: 49 provides a design for a nucleic acid sequence with CDS trained to a 65.1% expression design level encoding of a Zm00001eb366090_P001 variant of a GA20ox3 protein of Zea mays.

SEQ ID NO: 50 provides a design for a polynucleotide sequence to help identify what needs DNA editing with CDS trained to a 65.1% expression design level encoding of a Zm00001eb366090_P001 variant of a GA20ox3 protein of Zea mays.

SEQ ID NO: 51 provides the amino acid sequence of the Zm00001eb284010_P001 variant of an ZAG1 protein of Zea mays.

SEQ ID NO: 52 provides a design for a nucleic acid sequence with CDS trained to a 100% expression design level encoding of a Zm00001eb284010_P001 variant of a ZAG1 protein of Zea mays.

SEQ ID NO: 53 provides a design for a nucleic acid sequence with CDS trained to a 0% expression design level encoding of a Zm00001eb284010_P001 variant of a ZAG1 protein of Zea mays.

SEQ ID NO: 54 provides the nucleic acid sequence of the Zm00001eb284010_T001 CDS encoding of the Zm00001eb284010_P001 variant of a ZAG1 protein from Zea mays.

SEQ ID NO: 55 provides a design for a nucleic acid sequence with CDS trained to a 50.3% expression design level encoding of a Zm00001eb284010_P001 variant of a ZAG1 protein of Zea mays.

SEQ ID NO: 56 provides a design for a nucleic acid sequence with CDS trained to a 49.3% expression design level encoding of a Zm00001eb284010_P001 variant of a ZAG1 protein of Zea mays.

SEQ ID NO: 57 provides the polynucleotide sequence of the coding strand of the DNA region transcribed then spliced to the Zm00001eb284010_T001 transcript with CDS encoding of a Zm00001eb284010_P001 variant of a ZAG1 protein of Zea mays.

SEQ ID NO: 58 provides a design for a polynucleotide sequence to help identify what needs DNA editing with CDS trained to a 50% expression design level encoding of a Zm00001eb284010_P001 variant of a ZAG1 protein of Zea mays.

SEQ ID NO: 59 provides a design for a polynucleotide sequence to help identify what needs DNA editing with CDS trained to a 30% expression design level with putative encoding of a Zm00001eb284010_P001 variant of a ZAG1 protein of Zea mays.

SEQ ID NO: 60 provides a design for a polynucleotide sequence to help identify what needs DNA editing with CDS trained with Zm00001eb284010_T001 UTR-sequence-and-structure constraints to a 50% expression design level with putative encoding of a Zm00001eb284010_P001 variant of a ZAG1 protein of Zea mays.

SEQ ID NO: 61 provides a design for a polynucleotide sequence to help identify what needs DNA editing with CDS trained with Zm00001eb284010_T001 UTR-sequence-and-structure constraints to a 30% expression design level with putative encoding of a Zm00001eb284010_P001 variant of a ZAG1 protein of Zea mays.

SEQ ID NO: 62 provides the amino acid sequence of an HEX protein of Homo sapiens.

SEQ ID NO: 63 provides the nucleic acid sequence of a HEXA CDS encoding a variant of a HEX protein from Homo sapiens.

SEQ ID NO: 64 provides a design for a nucleic acid HEXA CDS sequence trained to a 100% expression design level encoding a variant of a HEX protein from Homo sapiens.

SEQ ID NO: 65 provides the nucleic acid sequence of a HEXA 5′ untranslated region of Homo sapiens.

SEQ ID NO: 66 provides the nucleic acid sequence of a HEXA 3′ untranslated region of Homo sapiens.

SEQ ID NO: 67 provides a design for a nucleic acid HEXA mRNA sequence with CDS trained to a 100% expression design level encoding a variant of a HEX protein of Homo sapiens.

SEQ ID NO: 68 provides a design for a nucleic acid HEXA mRNA sequence trained with HEXA UTR-sequence constraints to a 100% expression design level encoding a variant of a HEX protein of Homo sapiens.

SEQ ID NO: 69 provides a design for a nucleic acid mRNA sequence trained from Homo sapiens reference HEXA mRNA under UTR-sequence-and-structure constraints to a higher expression design level.

SEQ ID NO: 70 provides a nucleic acid sequence scenario with CDS encoding a variant of a putatively pathogenic V50M mutant TTR protein of Homo sapiens.

SEQ ID NO: 71 provides the reverse complement of a nucleic acid sequence scenario with CDS encoding a variant of a putatively pathogenic V50M mutant TTR protein of Homo sapiens.

SEQ ID NO: 72 provides a polynucleotide sequence of a doubly-minimal signature of a simulated V50M mutant TTR of Homo sapiens.

SEQ ID NO: 73 provides a polynucleotide sequence of a doubly-minimal signature of a simulated V50M mutant TTR of Homo sapiens.

SEQ ID NO: 74 provides a polynucleotide sequence of a doubly-minimal signature of a simulated V50M mutant TTR of Homo sapiens.

SEQ ID NO: 75 provides a polynucleotide sequence of a doubly-minimal signature of a simulated V50M mutant TTR of Homo sapiens.

SEQ ID NO: 76 provides a polynucleotide sequence of a doubly-minimal signature of a simulated V50M mutant TTR of Homo sapiens.

SEQ ID NO: 77 provides a polynucleotide sequence of a doubly-minimal signature of a simulated V50M mutant TTR of Homo sapiens.

SEQ ID NO: 78 provides a polynucleotide sequence of a doubly-minimal signature of a simulated V50M mutant TTR of Homo sapiens.

SEQ ID NO: 79 provides a polynucleotide sequence of a doubly-minimal signature of a simulated V50M mutant TTR of Homo sapiens.

SEQ ID NO: 80 provides a polynucleotide sequence of a doubly-minimal signature of a simulated V50M mutant TTR of Homo sapiens.

SEQ ID NO: 81 provides a polynucleotide sequence of a doubly-minimal signature of a simulated V50M mutant TTR of Homo sapiens.

SEQ ID NO: 82 provides a polynucleotide sequence of a doubly-minimal signature of a simulated V50M mutant TTR of Homo sapiens.

SEQ ID NO: 83 provides a polynucleotide sequence of a doubly-minimal signature of a simulated V50M mutant TTR of Homo sapiens.

SEQ ID NO: 84 provides a sequence for design of an antisense oligonucleotide corresponding to a doubly-minimal signature of a V50M mutated TTR of Homo sapiens.

SEQ ID NO: 85 provides a sequence for design of an antisense oligonucleotide corresponding to a doubly-minimal signature of a simulated V50M mutated TTR of Homo sapiens.

SEQ ID NO: 86 provides a sequence for design of an antisense oligonucleotide corresponding to a doubly-minimal signature of a simulated V50M mutated TTR of Homo sapiens.

SEQ ID NO: 87 provides a sequence for design of an antisense oligonucleotide corresponding to a doubly-minimal signature of a simulated V50M mutated TTR of Homo sapiens.

SEQ ID NO: 88 provides a sequence for design of an antisense oligonucleotide corresponding to a doubly-minimal signature of a simulated V50M mutated TTR of Homo sapiens.

SEQ ID NO: 89 provides a sequence for design of an antisense oligonucleotide corresponding to a doubly-minimal signature of a simulated V50M mutated TTR of Homo sapiens.

SEQ ID NO: 90 provides a sequence for design of an antisense oligonucleotide corresponding to a doubly-minimal signature of a simulated V50M mutated TTR of Homo sapiens.

SEQ ID NO: 91 provides a sequence for design of an antisense oligonucleotide corresponding to a doubly-minimal signature of a simulated V50M mutated TTR of Homo sapiens.

SEQ ID NO: 92 provides a sequence for design of an antisense oligonucleotide corresponding to a doubly-minimal signature of a simulated V50M mutated TTR of Homo sapiens.

SEQ ID NO: 93 provides a sequence for design of an antisense oligonucleotide corresponding to a doubly-minimal signature of a simulated V50M mutated TTR of Homo sapiens.

SEQ ID NO: 94 provides a sequence for design of an antisense oligonucleotide corresponding to a doubly-minimal signature of a simulated V50M mutated TTR of Homo sapiens.

SEQ ID NO: 95 provides a sequence for design of an antisense oligonucleotide corresponding to a doubly-minimal signature of a simulated V50M mutated TTR of Homo sapiens.

SEQ ID NO: 96 provides a sequence of a doubly-minimal DNA signature of a simulated V50M mutant TTR of Homo sapiens.

SEQ ID NO: 97 provides a polynucleotide sequence of a doubly-minimal DNA signature of a simulated V50M mutant TTR of Homo sapiens.

SEQ ID NO: 98 provides a polynucleotide sequence of a doubly-minimal DNA signature of a simulated V50M mutant TTR of Homo sapiens.

SEQ ID NO: 99 provides a polynucleotide sequence of a doubly-minimal DNA signature of a simulated V50M mutant TTR of Homo sapiens.

SEQ ID NO: 100 provides a polynucleotide sequence of a doubly-minimal DNA signature of a simulated V50M mutant TTR of Homo sapiens.

SEQ ID NO: 101 provides a polynucleotide sequence of a doubly-minimal DNA signature of a simulated V50M mutant TTR of Homo sapiens.

SEQ ID NO: 102 provides a polynucleotide sequence of a doubly-minimal DNA signature of a simulated V50M mutant TTR of Homo sapiens.

SEQ ID NO: 103 provides a polynucleotide sequence of a doubly-minimal DNA signature of a simulated V50M mutant TTR of Homo sapiens.

SEQ ID NO: 104 provides a polynucleotide sequence of a doubly-minimal DNA signature of a simulated V50M mutant TTR of Homo sapiens.

SEQ ID NO: 105 provides a polynucleotide sequence of a doubly-minimal DNA signature of a simulated V50M mutant TTR of Homo sapiens.

SEQ ID NO: 106 provides a polynucleotide sequence of a doubly-minimal DNA signature of a simulated V50M mutant TTR of Homo sapiens.

SEQ ID NO: 107 provides a polynucleotide sequence of a doubly-minimal DNA signature of a simulated V50M mutant TTR of Homo sapiens.

SEQ ID NO: 108 provides a polynucleotide sequence of a doubly-minimal DNA signature of a simulated V50M mutant TTR of Homo sapiens.

SEQ ID NO: 109 provides a polynucleotide sequence of a doubly-minimal DNA signature of a simulated V50M mutant TTR of Homo sapiens.

SEQ ID NO: 110 provides a polynucleotide sequence of a doubly-minimal DNA signature of a simulated V50M mutant TTR of Homo sapiens.

SEQ ID NO: 111 provides a polynucleotide sequence of a doubly-minimal DNA signature of a simulated V50M mutant TTR of Homo sapiens.

SEQ ID NO: 112 provides a polynucleotide sequence of a doubly-minimal DNA signature of a simulated V50M mutant TTR of Homo sapiens.

SEQ ID NO: 113 provides a polynucleotide sequence of a doubly-minimal DNA signature of a simulated V50M mutant TTR of Homo sapiens.

SEQ ID NO: 114 provides the nucleic acid sequence of the TTR CDS encoding a variant of TTR protein from Homo sapiens.

SEQ ID NO: 115 provides a polynucleotide sequence of a minimal on-site locus signature of a putatively non-pathogenic TTR of Homo sapiens.

SEQ ID NO: 116 provides a polynucleotide sequence of a minimal on-site locus signature of a putatively non-pathogenic TTR of Homo sapiens.

SEQ ID NO: 117 provides a polynucleotide sequence of a minimal on-site locus signature of a putatively non-pathogenic TTR of Homo sapiens.

SEQ ID NO: 118 provides a polynucleotide sequence of a minimal on-site locus signature of a putatively non-pathogenic TTR of Homo sapiens.

SEQ ID NO: 119 provides a polynucleotide sequence of a minimal on-site locus signature of a putatively non-pathogenic TTR of Homo sapiens.

SEQ ID NO: 120 provides a polynucleotide sequence of a minimal on-site locus signature of a putatively non-pathogenic TTR of Homo sapiens.

SEQ ID NO: 121 provides a polynucleotide sequence of a minimal on-site locus signature of a putatively non-pathogenic TTR of Homo sapiens.

SEQ ID NO: 122 provides a polynucleotide sequence of a minimal on-site locus signature of a putatively non-pathogenic TTR of Homo sapiens.

SEQ ID NO: 123 provides a polynucleotide sequence of a minimal on-site locus signature of a putatively non-pathogenic TTR of Homo sapiens.

SEQ ID NO: 124 provides a polynucleotide sequence of a minimal on-site locus signature of a putatively non-pathogenic TTR of Homo sapiens.

SEQ ID NO: 125 provides a polynucleotide sequence of a minimal on-site locus signature of a putatively non-pathogenic TTR of Homo sapiens.

SEQ ID NO: 126 provides a polynucleotide sequence of a minimal on-site locus signature of a putatively non-pathogenic TTR of Homo sapiens.

SEQ ID NO: 127 provides a polynucleotide sequence of a minimal on-site locus signature of a putatively non-pathogenic TTR of Homo sapiens.

SEQ ID NO: 128 provides a polynucleotide sequence of a minimal on-site locus signature of a putatively non-pathogenic TTR of Homo sapiens.

SEQ ID NO: 129 provides a polynucleotide sequence of a minimal on-site locus signature of a putatively non-pathogenic TTR of Homo sapiens.

SEQ ID NO: 130 provides a polynucleotide sequence of a minimal on-site locus signature of a putatively non-pathogenic TTR of Homo sapiens.

SEQ ID NO: 131 provides a polynucleotide sequence of a locus signature flanking the V50M variant site of TTR of Homo sapiens.

SEQ ID NO: 132 provides a polynucleotide sequence of a locus signature flanking the V50M variant site of TTR of Homo sapiens.

SEQ ID NO: 133 provides a design for a mRNA sequence with CDS trained to a 100% expression design level encoding a variant of a TTR protein from Homo sapiens.

SEQ ID NO: 134 provides the amino acid sequence of an TTR protein of Homo sapiens.

SEQ ID NO: 135 provides a design for a mRNA sequence trained from Homo sapiens reference TTR mRNA under UTR-sequence-and-structure constraints to a higher expression design level.

SEQ ID NO: 136 provides the amino acid sequence of an HBB protein of Homo sapiens.

SEQ ID NO: 137 provides the mRNA sequence encoding an HBB protein from Homo sapiens.

SEQ ID NO: 138 provides a design for a mRNA sequence trained under UTR-sequence constraints from Homo sapiens reference HBB mRNA to a higher expression design level.

SEQ ID NO: 139 provides a design for a mRNA sequence trained under UTR-sequence-and-structure constraints from Homo sapiens reference HBB mRNA to a higher expression design level.

SEQ ID NO: 140 provides the TP53 mRNA sequence encoding a variant of a p53 protein from Homo sapiens.

SEQ ID NO: 141 provides a nucleic acid sequence scenario with CDS encoding a variant of a putatively pathogenic R337H mutant p53 protein of Homo sapiens.

SEQ ID NO: 142 provides a sequence for design of an antisense oligonucleotide sequence corresponding to a doubly-minimal signature of a simulated R337H mutated p53 of Homo sapiens.

SEQ ID NO: 143 provides the polynucleotide sequence of a TP53 5′ untranslated region of Homo sapiens.

SEQ ID NO: 144 provides the polynucleotide sequence of a TP53 3′ untranslated region of Homo sapiens.

SEQ ID NO: 145 provides a design for a mRNA sequence trained under UTR-sequence-and-structure from Homo sapiens reference TP53 mRNA constraints to a higher expression design level.

SEQ ID NO: 146 provides a design for a mRNA sequence trained under UTR-sequence-and-structure constraints from Homo sapiens reference TP53 mRNA to a higher expression design level and fortified against an R337H ASO design.

SEQ ID NO: 147 provides a PKD2 mRNA scenario with a pair of nucleic acid sequences representing synonymous putatively non-pathogenic variants via IUPAC variant codes encoding a PKD2 protein from Homo sapiens.

SEQ ID NO: 148 provides a sequence for design of an antisense oligonucleotide corresponding to a doubly-minimal signature of a simulated synonymous putatively non-pathogenic variant encoding a PKD2 of Homo sapiens.

### DETAILED DESCRIPTION OF THE INVENTION

Proteins and (poly) peptides have many different functions in biological organisms that include but are not limited to catalysis of reactions as enzymes, transporters of molecules, and cellular structures. The presence and relative abundance of specific proteins plays an important role in organismal molecular biology and changing the presence and relative abundance of specific analogous proteins can be applied to improve human health, animal health, crop health, carbon capture, agricultural productivity and protection, and/or biologic activity. However, precisely affecting phenotypic changes and outcomes is expensive and error prone. And precisely changing expression of one or more analogous proteins in an organism has been challenging due to limited ability to control experimental factors, limitations of measurement, the exponential number of ways to affect gene expression of a (poly) peptide, and the limited precision and accuracy in predicting changes to expression when modifying transcription factors, promoters, and GC content. This disclosure introduces methods of gene training to help enable more direct, expedient, and successful product development pipelines to digitally adjust or aim expression level of one or more (poly) peptides for improving crops, carbon capture, biologics, research and development, and human health.

### A. Agriculture

With a ballooning world population, the associated demand for food is expected to increase by 50% by 2050, and with increasing scarcity of limited resources such as water, land, and fuel, there is a need to improve crops, carbon capture, livestock, and biologics to provide more value while using less.

From the transition from hunting and gathering, to cultivation of seeds that produced more, and to modern agriculture, methods of increasing sophistication have been ideated and put into production that increase crop productivity. Historical efforts to improve crops have led to directed breeding, hybrid crops, higher planting densities, weed and pest control, and greater productivity that supplies billions with food, fuel, and fiber. And in modern agriculture increasingly sophisticated analytical tools have been created to find and leverage useful genetic variation.

Tools such as genomic prediction and QTL analysis and GWAS are used to guide breeding recombination designs. However, a challenge in directed breeding has been that variation identified as beneficial with such tools does not always perform additively when applied in combination with other variation.

Modern analyses have not only become more sophisticated at screening and detecting variation for utility, but modern analyses have also significantly widened the search space by scouring the Earth for new and interesting species and genes that may prove useful for improving medicine, crop genetics, carbon capture, and livestock.

In addition to breeding, another approach, the biotech trait approach, was developed to make it possible to leverage global genetic variation that could not otherwise be used through breeding. Having identified genes with unique and useful properties, the biotech approach to gene insertion also helped facilitate further productivity gains via the transfer of genes from other species to add capability to the target genome. Use of alternative gene promoters has also been used to experimentally change the context of expression of a gene by changing which transcription factors initiate transcription (for example to express in a particular tissue or cell type) or to experimentally change expression itself by changing the degree to which transcription factors can bind to DNA to initiate transcription.

Additional biotech tools for complementing gene insertion that are used towards improving candidate gene constructs include tools to introduce codon-augmenting perturbations. Codon-augmenting perturbations are used to change encoded protein sequences towards improving protein function. However, codon-augmenting perturbations have also been used to experimentally change expression through trial and error, by enriching GC content and modifying codon frequencies to reflect those found in a target genome.

And with an understanding that genetic variation is of great importance to continued crop productivity increases, irradiation, said to be capable of causing gene variations much like the sun's rays do over millennia or eons, has also been used as a method of direct genome modification.

Site-Directed DNA editing tools, for example for gene modification (e.g. CRISPR) and for replacement (e.g. RMCE) have existed for more than a decade genome and can be used to improve genomes. Directed DNA editing tools have been used to ensure the precise location of genome changes, for example to replace one biotech trait protein with another newer biotech trait protein, to introduce so-called nonsense mutations, to turn off genes that impact crop performance, and even to undo a genetic mutation to cure sickle cell disease ex vivo.

These breeding and biotech tools have driven the discovery and application of performance-improving variation through use of global germplasm, the breeders' equation, and genome engineering to create new products has been incredibly successful together with better agronomic practices at increasing productivity several fold over the last one hundred years, for example in corn, from around thirty (30) bushels per acre to around one-hundred eighty (180) bushels per acre.

However, creating phenotypic improvements necessary to bring a new product to market is said to be extremely expensive. State-of-the-art biotech-based phenotypic modulation is done by creating a panel of polynucleotide constructs that have different promoters and/or varied GC content and/or encode protein variants, then creating many genomic augmentation events using genome engineering tools, and then screening each event individually within the context of a genome. It is not atypical for there to be hundreds or more attempts and multiple stages and gates to adjust a single protein and/or its expression to obtain the desired performance when applying the biotech trait approach. And in aggregate between the biotech and breeding approaches, a seed company's research stations can conduct as many or more than two million pollinations per year towards screening newly found genes and genomes in the hopes of finding and/or leveraging performance-improving variations [U.S. Pat. No. 5,811,639A].

The inexact, empirical, and cryptic nature of performance modulation has meant that adding an extra copy or turning off existing genes and introducing existing genes of other species represents the state-of-the-art of intersection of biotechnology and breeding today. As a result, breeding and/or development of biotech traits to produce superior genetics that achieve improvements in crops, carbon capture, livestock, and/or biologics has remained an expensive and challenging problem.

### B. Human Health

US healthcare spending is close to five trillion dollars annually, currently two fifths of which is paid for by the federal government. Pharmaceutical consumption alone has reached $600 billion annually in the United States. And pharmaceuticals are not just expensive for consumers, the development of pharmaceuticals is expensive. It is said that the average cost associated with developing a new pharmaceutical is over a billion dollars. And it is not unheard of for the resulting products to have side effects and drug interactions.

Gene therapies are a promising new approach to medicine, with three main approaches to developing gene therapies include genome modification, gene interference, and gene replacement. Genome modification such as through gene editing is a technique of growing importance that is intended to help address genetic disease by changing the recipient's genetics. However, addressing genetic disease with gene editing or gene insertion is considered by some to be risky due to the approaches' permanence and possibilities for introducing permanent genetic changes that end up being undesired or off-target. And genome modifications done through editing DNA can alter the context of transcription or disrupt the expression of other genes.

With gene interference and gene replacement therapies, the process to create a gene therapy requires one to pick a modality and create a design for a gene therapeutic for potential development. And once a method is selected, for example to develop antisense oligonucleotides (a type of gene interference therapy), additional analysis and experimentation is required to find a specific antisense oligonucleotide sequence that is both safe and effective.

However, despite the large amount of available information and analysis, many gene therapies have not achieved or sustained desired health benefits. And the development of gene therapies has remained largely elusive, with only one in five gene therapies receiving approval. Development of better medicines and better pipelines that increase success rate, including gene therapies, has remained a challenging problem and an ongoing need.

### C. About the Descriptions and Examples

In the descriptions that follow, for purposes of explanation, specific details are set forth in order to provide an understanding of the disclosure. One skilled in the art will recognize that embodiments of the present disclosure, described below, may be implemented in a variety of ways, such as a process, an apparatus, a system, a device, or a method on a tangible computer-readable medium, on a cpu, gpu, or other computational device or cloud, but some elements may not necessarily require a computer to implement. Components, or modules, shown in diagrams are illustrative of example embodiments of the disclosure and are meant to avoid obscuring the disclosure.

It shall be understood that when we refer to a peptide or protein, we mean the polypeptide produced by translation from mRNA. And when we refer to a polypeptide we mean a chain of amino acids of linked by peptide bonds that includes all of or a contiguous portion of a protein. It shall also be understood that when we refer to a “sequence” or “sequence design” or the like, that we are referring to a digital data string that may represent but has not necessarily been implemented into a potential biological counterpart. It shall be understood that when we refer to a “subsequence” we mean a contiguous portion of a sequence. It shall also be understood that while many of the example sequence designs do preserve intronic region sequences, preservation of intronic region sequences is not necessary to practice an embodiment of the invention. It shall also be understood that bioinformatics data such as polynucleotide sequences or polypeptide sequences found in but not limited to data, data files, and data file content referenced herein, can be considered to have been cleansed of line breaks and/or other artifacts or obscuring formatting found in the data representing bioinformatics sequences prior to analysis, and that reference in the disclosure to a sequence refers to the bioinformatics sequence and not necessarily in the format the raw bioinformatics data is found, to avoid obscuring the disclosure. Similarly, it shall be understood by one skilled in the art that when we refer to RNA, we mean one or more types of RNA, including but not limited to RNAs of specific origin or so-called maturity (e.g. mRNA from a primary transcript, rRNA from a ribosome, etc. . . . ), RNAs of a diversity of lengths (e.g. so-called small or long RNAs), RNAs purposes (e.g. interfering, transfer RNAs), contiguous portions of mRNA such as coding regions, and RNAs that have or have not been edited, for example by polyadenylation or other natural or human-directed means. In examples of this disclosure, the nucleotides adenine, cytosine, guanine, thymine, and uracil are represented respectively by letters A, C, G, T, U. It shall be understood that in general, T's found in DNA of a coding strand and are converted to U's when transcribed from the template strand or subsequently spliced to RNA, and we may in some circumstances refer to T's and U's interchangeably for convenience of communication, and substitution of U's for T's or T's for U's is sometimes done to expedite sequence searches and indexing.

It shall also be understood, that the methods and examples presented here are to be used also with other techniques. For example, the methods and examples provided here for agricultural use are to be used together with other methods to select genes for expression redesigns, gene-editing tools and other genome engineering techniques that effectuate genetic augmentation, and may require additional designs of guide RNAs, use of laboratory equipment, growth chambers, greenhouses, and other facilities or instrumentation, breeding tools and techniques, and due experimentation, to implement gene designs that can be produced with disclosed method(s), to evaluate the gene expression candidates like, but not limited to, those of the examples.

It shall be understood that the methods and examples of this disclosure are applicable to other phenotypes. It shall be understood that the methods of this disclosure when used for agriculture are to be used to influence phenotypes, which include, but are not be limited to, florescence traits, size, height, width, length, weight, volume, density, count, angle, growth rate, sugar content or production, starch content or production, oil content or production, fat content or production, protein content or production, hormone production, vitamins or other nutrient content or production, water use efficiency, nitrogen use efficiency, biotic disease resistance, pest resistance, herbicide tolerance, salt tolerance, temperature tolerance, and other traits deemed of value to, for example but not limited to, farmers, consumers, producers, seed companies, governments, organizations, and society.

It shall be understood that the methods of this disclosure when used for agriculture can be used to influence phenotype of a wide variety of organisms such as but not limited to livestock animals such as cattle, sheep, pigs, poultry, fish; agricultural crops such but not limited to grains, legumes, seeds, vegetables, fruits; when used in combination with genome engineering and/or gene augmentation techniques such as but not limited to base editing, prime editing, recombinase mediated cassette exchange to carry out processes such as including gene editing and gene replacement. It shall be understood that grains include but are not limited to maize, sorghum, rice, wheat, rye, oats, barley, millet. It shall be understood that legumes include but are not limited to soybeans, chickpeas, lentils, fava beans, peanuts. It shall be understood that seeds include but are not limited to oil seeds like rapeseed, canola, safflower, sunflower, flax seed, hemp seed, mustard. It shall be understood that vegetables and fruits include but are not limited to roots, corms, bulbs, rhizomes, tubers, leaves, stems, flowers and flower parts, buds, pods, seeds.

It shall be understood that algorithms, solvers, simulations, experimentation, and techniques from systems biology, plant and animal breeding, and operations research are all mechanisms that may assist with determining both what genes can be used as control mechanisms and what may be desired relative expression design levels to select with respect to a product concept, set of traits, and/or environment.

It shall be understood that breeding techniques, such as QTL studies or GWAS may identify a set of transcripts as important in the development of a particular trait. Using the techniques of breeding may also identify the directionality of effects (advantages or disadvantages) of variation in the set of transcripts. Simulation techniques, such as systems biology or operations research, may also identify constraints or bottlenecks in metabolism that can be widened or narrowed as desired to affect target traits.

It shall be understood that reference in the disclosure to “group testing”, “pooled testing”, or the like shall refer to one or more techniques for identifying which items of a set are important (or unimportant) to an application or need. Group testing methods shall be understood to include but not be limited to various probabilistic group testing, combinatorial group testing techniques, or subset finding techniques.

It shall be understood that the methods and examples of designs of medicinal polynucleotides for human health are to be used together with nucleic acid production techniques, medicinal formulation, clinical systems, due experimentation, and other techniques as well as with other data such as an individual's or a subpopulation's genomes, transcriptomes, and/or exomes and the medicinal polynucleotides can be redesigned using the disclosed methods for application to a specific individual and/or subpopulation using different data, formulation, and/or algorithmic augmentations.

It shall be understood that, to be effective at treating or preventing a genetic disease, multiple and/or distinct medicinal polynucleotides may be required, and those polynucleotides may focus on one or more genes and/or gene variants, and may require the same or separate delivery mechanisms or encapsulations. Additionally, it shall be understood that the treatment of multiple genetic diseases may also leverage the same or separate delivery mechanisms or encapsulations, and that autologous specificity, binding specificity and binding strength to proteins such as transporters, aversion of interaction with off-target proteins, or other properties of capsule decorations, may aid the proper delivery of one or more medicinal polynucleotides.

Also, the methods and example designs of sequences for medicinal polynucleotides are to be or can be used together with other methods to aid with formulation, efficacy, half-life, immune bypass, tissue and/or cell specific delivery, induction or control of protein expression, and other useful medicinal properties, and those methods may include but are not limited to the use of deep learning, machine learning, artificial intelligence, or other tools or calculations, such as but not limited to tools for protein folding, protein design, and protein augmentation/optimization.

Furthermore, medicinal polynucleotides must be formulated and/or evaluated for medical use, and typically require further formulation and due experimentation to enable delivery of the polynucleotides in proper dosages to the proper tissues, cells, and/or cell localities to be safe and effective, and that improper formulation, improper dosage, and/or improper locality of tissue/cell delivery can lead to serious side effects, additional disease, or death, and formulation needs can vary between genetic diseases and between individuals, depending on the nature of the disease, specific variant, and an individual's own genetics/transcriptomics.

It shall be understood that expression levels are expected to vary between organisms, within organisms, between tissues, between cell types, across developmental stages, and at different times within stages, times of day, organismal or gender-specific cycles or maturation phase, and in response to external factors and inputs including but not limited to food, O₂, CO₂, water, light, pH, temperature, environment, concentrations and/or availability of nutrients, environmental substances, or stressors, and that measurement of expression level is often obscured by such micro, macro, and other poorly, difficult, or generally not controlled or perhaps even impossible to completely or precisely control factors.

It shall be understood that there are additional biomolecules and/or factors that impact, increase, decrease, repress, initiate, or halt transcription and splicing of RNA and/or translation to proteins in various tissues and cell types, and that when affecting expression levels that it is generally, but not always, undesirable to override the natural course and context of in vivo expression (excepting that which is specifically intended by some genetic therapies), and that when aiming abundance of RNAs and/or expression level of proteins it is generally reasonable to keep intact, and not override the cell type by altering, the mechanisms that increase, decrease, repress, initiate, halt, or otherwise impact the timing or locality of expression of the targeted and untargeted RNAs and/or proteins (excepting traits such as including florescence or flowering time).

It shall be understood that measuring expression levels experimentally is often an imprecise endeavor, and that reference in the disclosure to “expression”, “expression level”, “relative expression level”, or the like refers to one or more notion(s) of (sometimes latent or aggregate) abundance (all else equal), and not simply to empirically measured value(s) that can be expected to have large and often non-deterministic or not (well or fully) understood variations.

Similarly, it shall be understood that reference in the disclosure to “expression design”, “expression design level”, “relative expression design level”, or the like means a specification of one or more values that impact the degree of presence of mRNA, accumulated mRNA, and/or translation to encoded proteins including a percentage of what is possible or of interest, minimum free energy, and/or half-life, and not necessarily to empirically measured value(s), and that there are expected to be differences between specifications and measurements, just as there are many situations (in biology and beyond) where observations may not reflect specifications, due to numerous factors, some of which are mentioned above.

It shall be understood that a length-adjusted MFE is a minimum free energy of an RNA divided by length of the RNA. It shall also be understood that the expression design levels including MFE and half-life used in methods are estimated values using one or more computational methods, including using RNAFold.

Furthermore, it shall be understood that in the context of RNA gene therapies, half-life is one mechanism to influence desired abundance of encoded proteins and that there are other mechanisms that can and do influence abundance of proteins (but that half-life differences can affect RNA abundance when all else is equal, including dosing and frequency). In addition, it shall be understood that in the context of a genome, transcribed mRNAs' half-lives affect the relative abundance of mRNA and thereby may affect the relative abundance of encoded proteins. It shall be understood that in the context of the disclosure “cellular” or the like means pertaining to a biological cell. Also, it shall be understood that in the context of the disclosure, “portion” or the like means one or more, not necessarily contiguous, parts. It shall be understood that CDS sequences and coding region RNA sequences are sometimes referred to interchangeably, with T's and U's intended according to context. Similarly, it shall be understood that CDNA sequences and mRNA sequences are sometimes referred to interchangeably, with T's and U's intended according to context.

It shall also be understood that throughout this disclosure that components may be described as separate functional units, which may comprise sub-units, but those skilled in the art will recognize that various components, or portions thereof, may be divided into separate components or may be integrated together, including, for example, being in a single system or component. It should be noted that functions or operations discussed herein may be implemented as components. Components may be implemented in software, hardware, or a combination thereof. Furthermore, connections between components or systems within the figures are not intended to be limited to direct connections. Rather, data between these components may be modified, re-formatted, or otherwise changed by intermediary components. Also, additional or fewer connections may be used. It shall also be noted that any communication, such as a signal, response, reply, acknowledgement, message, query, etc., may comprise one or more exchanges of information.

Reference in the disclosure to “one or more embodiments”, “an embodiment”, “embodiments”, “an example”, “examples”, “illustration”, “illustrations”, or the like means that a particular feature, structure, characteristic, or function described in connection with the embodiment is included in at least one embodiment of the disclosure and may be in more than one embodiment. Also, the appearances of the above-noted phrases in various places in the specification are not necessarily all referring to the same embodiment or embodiments.

The use of certain terms in various places in the disclosure are for illustration and should not be construed as limiting. A service, function, or resource is not limited to a single service, function, or resource; usage of these terms may refer to a grouping of related services, functions, or resources, which may be distributed or aggregated. The terms “include”, “including”, “comprise”, and “comprising” and the like shall be understood to be open terms and any lists that follow are examples and not meant to be limited to the listed items. A “layer” may comprise one or more operations. The use of memory, database, information base, data store, tables, hardware, cache, and the like may be used herein to refer to a system component or components into which information may be entered or otherwise recorded.

### D. Introduction

Given the expensive nature of using the current state-of-the-art pipelines and methods for agriculture [Paterson, Ioannidis, Cooper et al, Kelly, Halliburton, U.S. Pat. No. 5,811,639A] and gene therapies, what are needed are systems and methods that increase the efficiency and success rate of product development without as much screening and guesswork. This disclosure improves the state-of-the-art by describing product development pipelines that can increase the efficiency and success rate of product development, and significantly reduce the need for screening and guesswork. More specifically, the new product development pipeline designs more efficiently and effectively allow for the research and production of better agricultural products as well as higher success rates for gene therapy products intended to improve human health.

The current state-of-the-art biotech techniques (e.g. promoter substitution [Engstrom], amino acid perturbation [Wang et al], GC content modulation [Presnyak], genome-wide codon frequency alignment [U.S. Pat. No. 8,697,359 B1]), while helpful tools for genetic engineering, do have limitations and introduce performance uncertainties when attempting to improve genes and their genomes' phenotypic outcomes. What is needed are methods that precisely aim expression half-life of mRNAs encoding proteins of interest to reduce the need for additional analysis and experimentation while mitigating unnecessary off-target gene expression changes.

Without unnecessarily replacing targeted proteins with different proteins, the methods described in this disclosure improve the state-of-the-art by: controlling relative expression of proteins by precisely aiming mRNA half-lives, reducing the need for additional analysis and experimentation, helping mitigate the disruption of the performance of other genes and gene variants, and avoiding alteration of associated introns, untranslated regions (UTRs), or the genomic location or other known transcriptional or translational context.

The disclosed fluxual methods for gene training enables in silico expression assessment and sequence design to precisely aim RNA half-life and/or protein expression to specific levels to improve agriculture and to design new nucleic acid sequences, sequences for therapeutics, and/or sequences that do not require viral epitopes nor depend on an existing RNA therapeutic. One skilled in the art will recognize that fluxual gene training methods for affecting expression are not limited to finding a coding region sequence with maximum −MFE (e.g. CDSFold, LinearFold) [Terai et al., Zhang et al.] and are capable of generating an mRNA sequence with maximum −MFE that encodes a protein, under untranslated region sequence constraints. In addition, one skilled in the art will recognize that gene training is also capable of generating mRNA sequences with target expression level subject to UTR sequence and structure constraints. One skilled in the art will also recognize that the methods of this disclosure differ from the existing experimentally driven state-of-the-art in agriculture that modifies GC-content or changes codon frequencies to reflect codon frequencies of a target organism. Further one skilled in the art will recognize that the methods of this disclosure are not limited to maximization, minimization, or top k sequences and are designed to also produce sequences with desired expression level.

The disclosed antisense sequence design methods for gene training enables and improves in silico methods for designing antisense oligonucleotides. The in silico methods are designed to also reduce unnecessary interactions with non-target variants compared to the existing state-of-the-art in order to improve the safety and efficacy of therapeutics that leverage one or more ASOs. Furthermore, the disclosed methods for digitally designing sequences for ASOs improves the state-of-the-art by facilitating enumeration of a set of maximally-specific sequences for ASOs. Furthermore, the disclosed methods for digitally designing sequences for ASOs improves the state-of-the-art by mitigating binding to a combinatorial number of possible genotypes. When applied to design a suppressive mending treatment, the in silico methods work by reducing binding to non-target variants. When applied to design a multimodal mending treatment, the in silico methods are designed to work in tandem to reduce binding to non-target genes and non-target variants.

In agriculture, the disclosed fluxual gene training methods for aiming expression allow for previously unattained and more precise expression levels and maximal leverage of genomes' available protein designs (both native and non-native), awhile also facilitating creation of improved alternative or replacement polynucleotide sequences and/or constructs that encode variants of synthetically designed proteins, such as but not limited to experimental enzymes designed by Rosetta Fold Diffusion (RFDiffusion) [Watson et al.] and its successor(s).

In addition, the fluxual gene training methods described in this disclosure can be used to more directly improve the precision, direction, effectiveness, and expediency of discovering, creating, and leveraging variation to be used in agricultural performance-improving efforts. Together with the pipeline design disclosed, the fluxual gene training methods can be used to increase the success rate of creating performance-improving variation, to significantly improve the efficiency of development of agricultural improvements over existing screening approaches used today in breeding and biotech.

This disclosure also improves the state of the art of personalized healthcare. The mending treatment design process is disclosed that is designed to increase the safety and efficacy of nucleic acid therapeutic design and create more efficient development processes with a higher success rate of preventing, muting, delaying, or treating genetic disorders. The analytical process and use of specific phenotypic information together with an individual's and/or familial genotypic information are key design process enablers for developing one or more mending treatments for a prospective recipient, improving the state of the art over existing healthcare technology.

In human health, fluxual and antisense gene training methods can be used to create new gene therapies that supplement and/or suppress expression levels. When nucleic acid therapeutics are transcribable from a DNA-like structure [Bokobza et al.], the addition of a promoter may allow for the proper context of transcription, and the use of a tetracycline-dependent promoter [Gossen et al.][Yao et al.] may assist with the proper dosing. The gene training methods together with the pipeline disclosed reduces drug development risk and complexity by using specific types of clinical and/or familial phenotypic information together with genotypic information to produce a drug design recommendation for gene therapy consisting of recommended antisense oligonucleotides (ASOs) and/or supplemental mRNA(s) and any sequence fortifications to mitigate interactions, as well as human leukocyte antigens (HLAs) recommended for decoration to reduce undesired immune response and indicate the therapeutic to be considered so-called self. Optionally, an operator may add, select, and modify design elements such as the use of nucleotide analogs, adjustment of MFE (or design level), extension of ASO(s), fortifications, refinement of HLAs, and delivery mechanism (e.g. vesicle, LNP, polynucleotide structure), together with one or more transporter-specific protein decorations to guide the delivery of the polynucleotide(s) to the right tissue and/or cells [Dillard et al.][Bokobza et al.][Kaksonen et al.]. In the fluxual methods for gene training, a value, t, is used to design expression level. In some embodiments the value t is a percentage. In some embodiments the value t is a difference percentage from an originating sequence. In some embodiments the value t is or includes MFE. In some embodiments the value t is a half-life.

In some embodiments the originating sequence is a wild-type sequence. In some embodiments the originating sequence is a synthetic sequence. In some embodiments the synthetic sequence is a modified wild-type sequence. In some embodiments the originating sequence is a reference sequence that may or may not be a consensus of other sequences. In some embodiments the originating sequence has an estimated maximum −MFE. In some embodiments the originating sequence has an estimated maximal half-life. In addition, an extension of fluxual methods is disclosed that uses group testing, level lowering, and/or enumeration to aim (m) RNA half-life.

Gene training works by selecting a design level whereby sequences of polynucleotides are generated, wherein the designed polynucleotides are capable of affecting half-life or abundance of mRNA and expression of proteins (sometimes by transcription to mRNAs, other times through interference of mRNA). In some embodiments gene training identifies gene editing or gene replacement needs of DNA sequences such that the DNA with the designed sequence transcribes and/or splices (m) RNA encoding proteins of interest. In some embodiments, gene training can be used for the design of gene therapeutics that have specified half-life and/or help prevent, mitigate, mute, lessen, or treat a genetic disorder.

Gene training can be used together with transformation tools for gene insertion and construct design. In some embodiments, the desired expression level is presented or controlled on a scale for each protein that includes polypeptide(s) of interest. In some embodiments the desired expression level is specified as a percentage difference towards an extremal sequence. In some embodiments the desired expression level is specified as a percentage difference from an extremal sequence. In some embodiments the desired expression level is specified as a desired MFE. In some embodiments the desired expression level is specified as a desired half-life.

In some embodiments the scale is between 0-100%, where all-else-equal 0% represents a lower expression level and 100% represents a higher expression level. In some embodiments the scale is between −100% and 100% where all-else-equal −100% represents a lower expression level, 0% represents the expression level of a sequence intended to be used as an originating sequence, and 100% represents a higher expression level. In some embodiments the design levels may extend beyond the extremes of the scale, for example by adjusting half-life beyond the half-lives associated with the sequences having maximal or minimum MFE.

In some embodiments of the scale, the lower expression level represents the expression level of a sequence that has lower expression than desired. In some embodiments of the scale, the lower expression level is a minimal available expression level (all-else-equal).

In some embodiments of the scale, the higher expression level represents the expression level of a sequence that has higher expression than desired. In some embodiments of the scale, the higher expression level is a maximal available expression level (all-else-equal). In some embodiments of the scale, the higher expression level has a maximum −MFE over possible polynucleotides encoding a protein given two UTRs. In some embodiments of the scale, the higher expression level has a higher predicted half-life than a maximum −MFE polynucleotide.

In some embodiments where one is interested in increasing expression, the expression level of an existing polynucleotide sequence encoding a polypeptide of interest is used as the lower expression level in a scale. In some embodiments where one is interested in decreasing expression, the expression level of a polynucleotide sequence encoding a polypeptide of interest is used as the higher expression level in a scale.

Some example method embodiments for gene training are disclosed to generate a sequence that has the desired expression level, specified as a percentage of difference to implement with respect to a polynucleotide sequence. In other example method embodiments that work by group testing, level-lowering search, or enumeration, the desired expression level can also be specified as a half-life.

In agriculture, when it is necessary to edit or replace a gene in the context of a genome, the disclosed methods can help prioritize some DNA editing options over others, for example but not limited to reducing the length of the span of genome augmentation necessary when limiting, or prioritizing gene edits that are easier to make or require less expensive enzymes to implement.

In some embodiments, the methods of gene training enable the design of new pathogenicity-specific supplemental polynucleotides for autosomal recessive and more complex genetic disorders. In some embodiments the gene training enables the design of new pathogenicity-specific supplemental polynucleotides from a protein sequence together with flanking untranslated regions. In some embodiments the methods of gene training enable the design of new supplemental polynucleotides from consensus sequences and/or reference sequences. In some embodiments the methods of gene training enable the design of new supplemental polynucleotides from wild-type mRNA sequences with greater half-life over the wild-type mRNA and/or greater half-life over an mRNA whose coding region was not optimized in the context of flanking UTRs. In some embodiments the methods of gene training enable the design of new supplemental polynucleotides from mRNA sequences designed with CDSFold and/or a variant thereof and/or assembled together with flanking UTRs, or with decreased expression design level relative thereto.

And in the context of suppressive genetic medicines, the current state-of-the-art methods for in silico methods for designing antisense oligonucleotides currently report a range of possible lengths for antisense oligonucleotides, requiring extra effort and experimentation to arrive at a specific formulation. An ASO designed with more nucleotides than needed can have increased binding affinity to a non-pathological gene variant, which may cause negative side effects. An ASO designed with fewer nucleotides than needed can bind to sites unrelated to the pathological gene variant, which may also cause negative side effects. What are needed are in silico methods to maximize the binding specificity to the target gene variant of interest awhile avoiding binding to non-target or off-target polynucleotides. This disclosure also describes a second method that can be used for special cases of gene training for designing maximally-specific antisense oligonucleotides.

Furthermore, genetic medicines that only use a gene supplement to treat or prevent genetic disease without a suppressor may be less effective or ineffective. Similarly, genetic medicines that are designed to suppress a gene to treat or prevent genetic disease without supplementing a gene may be less effective, ineffective, or worse. What are needed are genetic medicines that are designed to best suit each individual, depending on the specific pathogenicity, and designed to reduce interference between suppressive and supplemental polynucleotides. In the context of designing gene therapies and personalized medicines, and using one or more computers, the disclosed methods are combined with specific phenotypic and sequence information to enable the disclosed pipeline to produce a drug design recommendation that reduces drug development risk and complexity.

The methods for gene training described in this disclosure are to be used to address the above-mentioned problems in designing new gene therapies to better assure specificity, help treat pathogenicity, improve efficacy, generate UTR-property-preserving mRNA with designed expression level, and individual-tailor treatments awhile averting unnecessary off-target side effects.

### E. Gene Training Methods-Aiming Expression Level

Two types of in silico design methods for gene training are disclosed here. Both types of methods are polynucleotide sequence design methods. We refer to the two types of gene training methods as fluxual and antisense. Fluxual gene training is for the design of polypeptide-inducing polynucleotide sequences. We say a polynucleotide is polypeptide-inducing if the polynucleotide directly or indirectly, for example through transcription and/or splicing, encodes one or more proteins that comprise the referent polypeptides. Antisense gene training is for the design of sequences for polynucleotides that inhibit production of one or more proteins that include polypeptides of interest.

Fluxual gene training can be used to tune mRNA half-life and accumulated expression of in-cell production of analogous proteins. Antisense gene training can be used to more expediently design antisense gene therapies.

In the descriptions that follow, the fluxual gene training methods are first disclosed, followed by disclosure of their applications to improve agriculture and agricultural pipelines. Then the antisense gene training methods are disclosed to improve existing antisense oligonucleotide design methods, followed by disclosure of gene training methods' applications to increase success rate of gene therapies and gene therapy pipelines to improve personalized healthcare.

### Fluxual Gene Training

Fluxual gene training methods are focused on in silico design of sequences for polynucleotides that produce proteins at specified levels. For example, in some example embodiments, given a protein sequence P with polypeptide(s) of interest, untranslated regions with necessary mobility properties, and a percentage t representing an expression level, fluxual gene training designs sequences for mRNA (and/or redesigns DNA wherein the DNA is capable of being transcribed and/or spliced into mRNA encoding the polypeptides) with expression level t.

Fluxual gene training is able to use a variety of different estimators, including but not limited to examples such as calculated difference percentage, −MFE of a coding region, estimates of −MFE of an mRNA and not just its coding region or a portion thereof, an estimate of half-life, production capacity of an mRNA for assessment of the mRNAs' relative ability to express an encoded protein. However, fluxual gene training may also use other estimators that assist therein. Let us denote an estimator as a function EL where S is an RNA sequence and EL(S) represents some estimate of interest for a polynucleotide of sequence S.

Fluxual gene training uses a parameter, t, for design of coding region sequences and/or mRNA sequences. In some embodiments the parameter value can be viewed as a difference percentage. In some embodiments the difference percentage can be viewed as the expression level between 0% to 100%. In some embodiments the difference percentage can be viewed as the degree of difference in expression level with respect to a polynucleotide sequence. In some embodiments, the parameter t can be viewed in the units of the supplied estimator. In some embodiments the units of the supplied estimator is MFE. In some embodiments the units of the supplied estimator is half-life.

The parameter value of t can be used to precisely control the accumulation of translatable mRNAs to affect the preferential translation of encoded polypeptides and their expression level relative to other polypeptides (all-else-equal). When t is in units of half-life and p represents a constant rate of production, p, of mRNA that decays exponentially, it can be deduced that the equilibrium accumulated abundance of mRNA (as time goes to infinity) can be described by Q(t,p), where Q(t,p)=t*p*log 2 (e). DegScore is an example in silico calculator [DegScore] of mRNA half-life [Mauger et al.].

Furthermore, given an existing equilibrium level of mRNA abundance, Q, and a new half-life h′, it is then possible to deduce that the new equilibrium mRNA abundance Q′=Q*h′/h. That is, a decrease (increase) in half-life by multiplier m, can be expected to decrease (increase) the abundance of mRNA also by multiplier m. One skilled in the art shall recognize that other factors can influence abundance of mRNA (such as but not limited to transcription factors and promoters mentioned before) and that abundance of mRNA need not be observable to influence phenotype. Furthermore, it is possible to deduce that protein production rate PPR is equal toQ* TR, where TR represents translation rate, where translation rate is the translation rate capacity times the percentage of the capacity supplied by ribosomes. Then, overall protein production may be considered equal to the integral of PPR over time.

In silico design of expression level by aiming t both contrasts with and complements the use of promoters because promoter changes can both directly impact the production rate of (m) RNA and impact which cells express which proteins, while aiming t can be used to aim the abundance of (m) RNA for a target protein without changing the production rate (i.e. transcription) nor changing the translation frequency of the (m) RNA molecule itself.

Given a polynucleotide sequence, S, other estimators may include, but are not limited to, various definitions of adjusted minimum free energy (e.g. MFE(S) divided by the length of S), half-life-related estimates such as in silico half-life from DegScore(S), in vitro half-life, levels or flows of metabolic substrate(s) or product(s), and other qualitative and quantitative properties of the polynucleotide of S, its splices and/or their coding regions, and/or respective primary transcript, and/or protoplast or organism containing the sequence, directly or indirectly, alone or in aggregate, via product or composition of functions, through measurement, phenotypic observation, calculation, and/or prediction.

In some embodiments, aiming by t can also be done to generate sequences for mRNA that have other important properties. Some example sequence properties of (m) RNA include what polypeptides are encoded and the arrangement of the polypeptides, the sequences of codons that encode the polypeptides, and untranslated regions (optionally inclusive of introns). Other properties of (m) RNA include structural properties, such as secondary structure, and consequential properties, such as mobility-related properties that help facilitate translational localization and cellular transport (e.g. export from the nucleus), and functionality-related properties such as binding characteristics and/or enzymatic classification(s) of induced polypeptide(s).

Let us refer to two polynucleotides that have the subsequences and conformational elements necessary for transport to equivalent cellular locality (optionally to escape the cellular nucleus) as mobility analogous. Let us refer to two biological sequences that include or induce polypeptides with the same function as functionally analogous. We say two polynucleotides are analogous if the two sequences are mobility analogous and also functionally analogous.

Fluxual gene training is capable of aiming expression design levels by generating sequences for polynucleotides that are capable of transcribing functionally analogous proteins. One can apply fluxual gene training by identifying a sequence for a protein with one or more polypeptides of interest, obtaining one or two polynucleotide sequences that encode the protein, and generating one or more new sequences for polynucleotides that induce functionally analogous proteins at the designed expression level, leveraging existing untranslated region sequences that target the desired subcellular localities and/or tools capable of untranslated region sequence design where needed. Desired subcellular localities can be targeted using one or more methods of determining or predicting subcellular localization, including but not limited to untranslated region sequence and/or secondary structure [Wang et al.][Engel et al.].

Six example difference-based approaches to affecting expression levels are shared here, five of which can be said to be capable of aiming expression levels in a genome (approaches 1, 3, 4, 5, 6). In the methods that are capable of aiming expression levels, gene training can be done as simply as specifying a target protein sequence, desired UTRs, and a value for the parameter t.

Alternatively, gene training can be done by specifying an (m) RNA sequence and a value for the parameter t when the UTRs, exons, and/or coding region are identified or identifiable.

In example methods, a coding region sequence and/or mRNA sequence is identified or generated (e.g. using composition); a second coding region sequence and/or mRNA sequence is identified or generated (optionally with UTR sequence constraints); and optionally a third coding region sequence and/or mRNA sequence is generated (optionally with UTR sequence and/or property constraints, including for example conformation, secondary structure constraints).

Note that the fluxual gene training methods can be used to generate (and/or find differences between) contiguous portion(s) of a sequence (two sequences). A contiguous portion can be described as an interval within a sequence. A contiguous portion can also be described as a subset PD of the difference D between two sequences such that the range of i's and range of j's of the pairs (i,j) in PD and the range of i's and range of j's of the pairs (i,j) in D-PD do not overlap. One skilled in the art will recognize that there are |D|-|PD| ways to define a contiguous partial difference when the difference is D and partial difference is PD.

In the context of editing genes (e.g. through genome engineering techniques that perform gene replacement), finding a contiguous partial difference with desired properties may help reduce editing time and complexity. However, one skilled in the art shall recognize that contiguousness of partial differences is not a requirement to practice fluxual gene training, because the methods can work with sequence constraints. Furthermore, in some circumstances it may be beneficial to avoid selecting differences near splicing sites if it is valuable to avoid altering splicing.

### Approach 1: The Dynamic Programming (DP) Approach

In a first example approach, a first sequence, S, is obtained that represents a polynucleotide that encodes a polypeptide P and a second sequence, T, is generated that represents a polynucleotide that encodes P. In some embodiments, all or a portion of the polynucleotide of S is an encoding of P. In embodiments where the polynucleotide of S is exactly an encoding of P, the polynucleotide of T also exactly encodes P.

In some embodiments, all or a portion of S is from one or more existing coding region sequences. In some embodiments, all or a portion of S is generated by tooling.

In some embodiments, the polynucleotide of S has one or more untranslated regions. In some embodiments, all or a portion of the untranslated region sequence(s) in S are from one or more existing mRNA sequence and/or pre-mRNA sequence. In some embodiments, all or a portion of untranslated region sequence(s) in S are generated by tooling. In some embodiments, tooling used to generate or help generate all or part of S includes a method for (re) design of untranslated region sequence(s). In one or more embodiments, the untranslated region(s) of the polynucleotide of S have required mobility-properties. In embodiments where S has one or more untranslated regions, T has the same untranslated regions.

In some embodiments, the tooling is capable of producing S such that EL(S) is maximal, maximum, and/or minimal. In some embodiments EL(S)=−MFE (CRP(S)), where CRP(S) denotes all or a portion of the coding region sequence of S. In some embodiments, EL(S)=−MFE(S).

In some embodiments, tooling used to generate or help generate all or part of S and/or T allows for additional constraints on all or a portion of the polynucleotide solution space. In some embodiments, the portion with constraints on generation includes the untranslated region(s). In some embodiments, the portion with constraints on generation includes part of the coding region(s).

In some embodiments, the tooling includes CDSFold or a variant thereof.

In some embodiments, the variant is a customized version of CDSFold that includes further extension of the Zuker approach by simultaneously including both amino acid and nucleotide constraints for the generation of a sequence. In an example implementation of the further extension and using the same notation as CDSFold (Nᵢ|n denoting the set of allowable nucleotides at position i after nucleotide n according to amino acid constraints and Nᵢ\hat{ }n denoting the set of allowable nucleotides at position i before nucleotide n according to amino acid constraints), we use mᵢ to denote the set of nucleotide constraints at position i:

$${F}^{{n}_{i} , {n}_{j}} ( i , j ) = min \{ \begin{matrix}{min}_{{n}_{i + 1} ∈ ( {m}_{i + 1} ⋂ ( {N}_{i + 1} \vert {n}_{i} ) )} [ {F}^{{n}_{i + 1} , {n}_{j}} ( i + 1 , j ) ] \\ {min}_{{n}_{j - 1} ∈ ( {m}_{j - 1} ⋂ ( {N}_{j - 1} ∧ {n}_{i} ) )} [ {F}^{{n}_{i} , {n}_{j - 1}} ( i , j - 1 ) ] \\ {C}^{{n}_{i} , {n}_{j}} ( i , j ) \\ {min}_{\begin{matrix}i < k < j , {n}_{k} ∈ ( {m}_{k} ⋂ {N}_{k} ) , \\ {n}_{k + 1} ∈ ( {m}_{k + 1} ⋂ ( {N}_{k + 1} \vert {n}_{k} ) )\end{matrix}} [ {F}^{{n}_{i} , {n}_{k}} ( i , k ) + {F}^{{n}_{k + 1} , {n}_{j}} ( k + 1 , j ) ]\end{matrix}$$

The choice of nucleotide constraints defined by mᵢ at each position i depends on the particular need or combination of needs. For each position i in a polynucleotide sequence, mᵢ is defined to be the set intersection of the additional nucleotide constraints.

For example, in embodiments where S at position i is required to match an untranslated region sequence at position j, the set intersection defining mᵢ includes {UTR[j]}. In another example, in embodiments where T[i] must equal S[i], the set intersection defining mᵢ includes {S[i]}. In another example, in some embodiments, the set intersection defining mᵢ includes a set of allowed nucleotide variants at position i. In another example, in some embodiments, the set intersection defining mᵢ includes a set of available and/or naturally occurring variation. For all mᵢ or mᵢ that do not need additional nucleotide constraints or synthetic or other nucleotides, the set intersection defining mᵢ includes a set of available nucleotides B (explicitly and/or implicitly), where B includes A,C,G,U.

In some embodiments, the portion of the polynucleotide sequence (not) only constrained by B is contiguous. In some embodiments, the portion of the polynucleotide sequence (not) only constrained by B is not contiguous.

To implement nucleotide constraints, one may also define a set of excluded codons for each position wherein nucleotide constraints are desired. The set of excluded codons for an amino acid position are therefore equal to the set of codons that encode the amino acid, wherein codon Cᵢ is in the set of excluded codons if Cᵢ[1]∩m₃ᵢ₋₂ is empty or Cᵢ[2]∩m₃ᵢ₋₁ is empty or Cᵢ[3]∩m₃ᵢ is empty.

In some embodiments, the portion of the coding region of T not needing explicit additional nucleotide constraints is determined approximately by the absolute value of a parameter t, where the remaining portion of T is composed to have same sequence(s) as the respective portion of S, where t is a percentage between −100% and 100%, where the sign of t indicates whether T is to be designed with decreased or increased expression level.

In some embodiments, a combination of the aforementioned embodiments is applied.

Let us denote DP-based fluxual gene training using the following notations G_{DP}(S,t), G_{DP}((N5,P,N3),t), G_{DP}(S,t,C), and G_{DP}((N5,P,N3),t,C), where S is an mRNA sequence or coding region sequence of polypeptide P, N5 and N3 are 5′ and 3′ polynucleotide sequences, t represents an expression level (e.g. percentage between extrema) or difference in expression level (e.g. percentage to extrema), and C represents additional constraints on the generation of T.

In some embodiments N5 equals U5 and N3 equals U3 are wherein U5 and U3 are 5′ and 3′ untranslated region sequences (respectively) from a (digital) library compatible with P. In some embodiments T is generated as a concatentation of results of multiple runs of the dynamic programming approach on a protein, wherein polypeptide P represents a contiguous portion of the protein.

### Approach 2: The Evolutionary Approach—May Extend Expression Range

In a second example approach, a second sequence T of an mRNA that includes an encoding of a protein P is generated from a first identified sequence, S, representing a polynucleotide that also includes an encoding of P such that: PROD(S)<PROD (T) or PROD(S)>PROD (T), there exists at least one position i such that T[i] and S[i] are unequal, and PROD is a function correlated with expected total protein production capacity of an mRNA molecule, or components thereof, for example PROD(S)=2*HL(S)*RC(S)/TT(S), where RC is an estimator of ribosomal capacity (the number of ribosomes that can co-translate on an mRNA molecule), HL determines mRNA half-life or an estimator thereof, and TT is mRNA translation time for an individual ribosome to translate an mRNA molecule, or an estimator thereof.

In some embodiments, the choice of number of positions where T[i] is chosen to be a nucleotide other than S[i] is done stochastically, via a parameter t, where t ranges between 0% and 100% and describes the percentage of the positions of T where T[i] is to be chosen to be a nucleotide other than S[i]. In some embodiments the choice of positions where T[i] is chosen to be a nucleotide other than S[i] is done stochastically. In some embodiments, the choice of nucleotide for T[i] when T[i] is chosen to be a nucleotide other than S[i] is done stochastically. An example application of the evolutionary approach for improving an existing RNA therapeutic is given in the following reference [Ser. No. 18/546,698].

One skilled in the art shall understand that this second example approach and embodiments are examples of an evolutionary algorithm. One skilled in the art shall also understand that evolutionary algorithms often have difficulty escaping local optima and have difficulty with finding or recognizing optimum.

In some embodiments an enumerative approach is applied wherein each synonymous codon at each position of a coding region sequence is tested for capability to extend the span of the range of available half-lives, and repeating if desired until no additional span extensions are possible.

A secondary approach may be suitable for increasing the span of the range(s) of attainable mRNA production rates when S originates from tooling that minimizes (or maximizes) a related but different measure (e.g. MFE), such as with one or more of the other approaches including but not limited to the dynamic programming approach described previously, and a half-life outside the bounds of MFE optimized sequences is needed. In some embodiments, all or a portion of S is identified from one or more existing RNA sequences. In some embodiments, all or a portion of S is identified from one or more sequences generated by tooling.

In some embodiments, generation of all or a portion of T is subject to constraints including but not limited to subsequence and/or conformation such as secondary structure as determined by a tool including but not limited to RNAFold. In some embodiments, T is generated by composition. S can also be altered to become T.

One skilled in the art shall understand that when circumstances require extended expression level extrema or range, a secondary algorithm such as an evolutionary algorithm may be substituted into or appended to the process. However, for brevity of disclosure and to avoid obscuring the disclosure, for the remainder of this disclosure this extra step can be understood to be available even when not explicitly denoted.

### Approach 3: The Difference-Based Approach

In a third example approach, two unequal sequences S and T, representing polynucleotides encoding the same protein P are obtained, wherein EL(S)>EL(T) and S is obtained prior to T, wherein a partial difference between S and T is used to generate a third sequence R representing a polynucleotide encoding P. In some embodiments the S and T polynucleotides have untranslated regions.

A sequence alignment, A, between two sequences S and T is a one-to-one mapping between a subset of the indices of S and a subset of indices of T. A sequence alignment can be represented as a set of paired indices (i,j), where i represents an index of the first sequence and j represents an index of the second sequence.

A sequence alignment, A, is defined to be order-preserving (or monotonicity-preserving) if there does not exist paired indices (i,j) and (i′,j′) in A such that i<i′ and j>j′. An order-preserving alignment is defined to be an identity alignment if and only if there exists an (i,j) in A for each position i of S and also for each position j of T.

With respect to an alignment, A, between sequences S and T, each index pair (i,j) of A can be considered not different if S[i] and T[j] are equal, and otherwise (i,j) can be said to be included in the difference.

In embodiments where the sequences are polynucleotide sequences aligned by an identity alignment, the difference includes a set of pairs of indices, each indice indicating nucleotide position in its polynucleotide sequence. In embodiments where the sequences are codon sequences aligned by an identity alignment, the difference includes a set of pairs of indices, each indice indicating codon position in its codon sequence.

In the differencing approach, the sequence, R, encoding P is generated by calculating a difference between S and T, selecting a subset D of the difference, selecting either S or T to designate as the primary bounding sequence (PBS), whereas the remaining sequence is designated the ultimate bounding sequence (UBS), and by using a method that implies, generates, or composes R from D and the PBS.

Here we note that when the difference between coding region sequences is defined at the nucleotide resolution, care can be required to not inadvertently change the corresponding amino acid when selecting a partial difference. For example, when applying an embodiment with the standard codon map, selection of a nucleotide difference in a codon encoding Serine, Leucine, Arginine, or STOP (S, L, R, and *, respectively) can require care to not inadvertently change the encoded protein. The care to be taken depends upon the PBS codon, the corresponding synonymous UBS codon, and the full set of synonymous codons for the applicable codon mapping. In some embodiments, care can be implemented as constraints.

In some embodiments, there are constraints on what can be included in D. In some embodiments, only pairs of indices may be included in D. In some embodiments, the constraints describe which pairs of indices may be included in D. In some embodiments, the constraints only indicate for a given D, whether a selected set of nucleotide index pairs may be included in D. For example in some embodiments where the difference is defined with respect to an identity alignment, a nucleotide index pair (i,j) may be included in D if and only if: S[i] is unequal to T[i], (i′,j′) is also included in D when S[i′] is unequal to T[j′], and position i′ has the same codon position and position in codon as position i in S and position j′ has the same codon position and position in codon as position j in T. Let us refer to the preceding example as the all-or-none-codon-nucleotide-differences example.

In some embodiments where a difference subset D is with respect to an identity alignment between S and T, R is implied, generated, or composed by the method described in Table 1: Implying R from originating sequence S and a subset D of the difference between S and T. Embodiments can also alter S or T to become R.

TABLE 1
Implying R from originating sequence S and a
subset D of the difference between S and T.

| | |
|---|---|
| R[i] = T[j] | if (i,j) in D |
| R[i] = S[i] | otherwise. |

In some embodiments, a target expression level, t, is given as percentage between 0% and 100%, wherein t represents approximately the percentage of a difference to be included in D.

In some embodiments, all or a portion of S and/or T originates from one or more existing RNA sequences. In some embodiments, all or a portion of S and/or T is generated by tooling. In some embodiments, generation of S and/or T is done such that EL(S) and/or EL(T) are minimized, maximized, or maximum. In some embodiments, the tooling includes CDSFold or a variant thereof. In some embodiments, EL(S) is a difference percentage. In some embodiments, EL(S)=−MFE (CRP(S)), where CRP(S) denotes all or a portion of the coding region of S. In some embodiments, EL(S)=−MFE(S). In some embodiments EL(S)=HL(S) where HL is a function that estimates the half-life of an mRNA sequence.

In some embodiments, the generation of all or a portion of S and/or T and/or R is done under constraints. In some embodiments a constraint for a position i of S and/or T and/or R is described via a set m; that further restricts the set of nucleotides or codons allowable at position i. For example, the generation of S and/or T may be constrained such that one or more codon positions of S and/or T are constrained to one or more specific amino acids. In another example, given sequences S and T and using the identity alignment between S and T, the codon at position i in R must be a codon that encodes for the same amino acid at as the codon at position i in S or T, even if the codon at position i in R is unequal to either of the codons at position i in S or T. Let us refer to this as the as-long-as-its-the-same-amino-acid-example. Please note that the all-or-none-codon-nucleotide-differences-example and the as-long-as-its-the-same-amino-acid-example differ in their flexibility of constraint on the generation of R.

In some embodiments, mᵢ is the result of a set intersection. For example, in embodiments where the nucleotide of position i of the generated sequence is required to be equal to the nucleotide of an untranslated region sequence at position j, the set intersection defining mᵢ includes {UTR[j]}. In another example, in embodiments where S[i] and/or T[i] must equal a nucleotide b, the set intersection defining mᵢ for generating S and/or T includes {b}. In another example, in some embodiments, the set intersection defining mᵢ includes a set of allowed nucleotides representing variants at position i. In another example, in some embodiments, the set intersection defining mᵢ includes a set of available and/or naturally occurring variation. For all mᵢ or mᵢ that do not need additional constraints or synthetic or other nucleotides, the set intersection defining mᵢ includes a set of available nucleotides B (explicitly and/or implicitly), where B includes A,C,G,U.

In some embodiments, the portion of the polynucleotide sequence (not) only constrained by B is contiguous. In some embodiments, the portion of the polynucleotide sequence (not) only constrained by B is not contiguous.

In some embodiments, a combination of the above embodiments is applied.

Let us denote difference-based fluxual gene training using the following notations G_{DB}(S,T,t) and G_{DB}(S,T,t,C) where S and T are either coding region sequences or mRNA sequences, t represents an expression level (e.g. percentage between extrema when S and T are considered to be relevant extrema) or difference in expression level (e.g. percentage to an extrema when only one of S or T are considered to be a relevant extremal), and C represents additional constraints on the generation of T.

### Approach 4: The One-Step Approach

It is worth pointing out at this point that the three preceding approaches use a percentage t in their parameterizations, where t represents a measure of difference from an originating sequence from which to generate a new sequence.

The follow approach uses the dynamic programming approach to provide a result in one-step when t is a percentage between −100% and 100%, representing approximately the desired percentage of difference from the current sequence with respect to maximal and maximum −MFE. The approach does not use structural constraints, but does allow for sequence constraints, is a generative one-step approach, and uses an existing mRNA and/or coding region polynucleotide sequence.

H_{DP}(S,t,C): //one step when t is a percentage between −100% and 100%
T = G_{DP}(S,t,C.sequence) //result

The following approach uses the dynamic programming approach to provide a result in one-step when t is a percentage between 0% and 100%, representing approximately the desired expression level on a scale between a minimal and maximum −MFE. The approach does not use structural constraints, but does allow for sequence constraints, is a generative one-step approach, uses a protein sequence and flanking untranslated region sequences as inputs, and does not require an existing polynucleotide coding region sequence to work.

```
H_{DP}((U5,P,U3),t,C): //one step when t is a percentage between 0% and 100%
  S = G_{DP}((U5,P,U3), 100%,C.sequence) //maximum −MFE mRNA
  T = G_{DP}(S,-t,C.sequence) //result
```

The follow approach uses the difference-based approach to provide a result in one-step when t is in the units of an expression level estimator representing approximately the desired expression level (e.g. a percentage, an −MFE, an adjusted −MFE, a half-life). The approach does not use structural constraints, but does allow for sequence constraints, is a generative one-step approach, and leverages two existing mRNA and/or coding region sequences as sequences with extremal expression levels.

```
H_{DB}(S,T,t,C): //one step when t is in units of EL (e.g. half-life, −MFE, or percentage)
  tₕᵢ = EL(S)
  tₗₒ = EL(T)
  tᵢₙ = (t-tₗₒ)/(tₕᵢ-tₗₒ)
  R = G_{DB}(S,T,tᵢₙ, C.sequence) //result
```

The follow approach uses the difference-based approach to provide a result in one-step when t is in the units of an expression level estimator representing approximately the desired expression level (e.g. a percentage, an −MFE, an adjusted −MFE, a half-life). The approach does not use structural constraints, but does allow for sequence constraints, is a generative one-step approach, and leverages an existing mRNA sequence or coding region sequence.

```
H_{DB}(S,t,C): //one step when t is in units of EL (e.g. half-life, −MFE, or
percentage)
  t_{cur} = EL(S)
  if t < t_{cur} then:
    T = G_{DP}(S,−100%,C.sequence) //maximal MFE mRNA
    tₗₒ = EL(T)
    tᵢₙ = (t−tₗₒ)/(t_{cur}−tₗₒ)
    R = G_{DB}(S,T,tᵢₙ,C.sequence) //result
  else:
    T = G_{DP}(S,100%,C.sequence) //maximal −MFE mRNA
    tₕᵢ = EL(T)
    tᵢₙ = (tₕᵢ−t)/(tₕᵢ−t_{cur})
    R = G_{DB}(T,S,tᵢₙ,C.sequence) //result
  Result = R
```

The following approach uses the difference-based approach to provide a result in one-step when t is in the units of an expression level estimator representing approximately the desired expression level (e.g. a percentage, an −MFE, an adjusted −MFE, a half-life). The approach does not use structural constraints, but does allow for sequence constraints, is a generative one-step approach, uses as inputs a protein sequence and untranslated region sequences for flanking the generated coding region, and does not require an existing polynucleotide coding region sequence to work.

```
H_{DB}((U5,P,U3),t,C): //one step when t is in units of EL (e.g. half-life, -MFE, or percentage)
  S = G_{DP}((U5,P,U3),100%,C) //maximal −MFE mRNA
  T= G_{DP}((U5,P,U3),0%,C) //maximal MFE mRNA
  t_{S} = EL(S)
  t_{T} = EL(T)
  tᵢₙ = (t-t_{T})/(t_{S}-t_{T})
  R = G_{DB}(S,T,tᵢₙ,C) //result
```

Approach 5: A Narrowing Approach-Constrained and/or Extra Precision Over One-Step

We refer to a fifth approach as a narrowing approach. The narrowing approach can in some situations increase precision over one-step approaches, especially when there is desire to aim expression with an alternative assessment function, or when it is desirable to ensure that newly produced polynucleotide sequence(s) have also the important properties (e.g. conformational) of the originating sequence, when the originating sequence is an mRNA sequence.

Embodiments of the narrowing approach can be considered to be level-lowering, enumeration, and/or group testing patterns, where level-lowering can provide extra precision quickly when not using structural constraints, enumeration can further provide extra precision and work under structural constraints, and group testing in some situations may improve speed to a result when working under structural constraints.

One skilled in the art shall recognize that decreases (or increases) in t do not strictly monotonically decrease (or increase) EL, especially considering the diversity of embodiments possible for EL including, but not limited to, −MFE, adjusted −MFE, half-life. However, one skilled in the art shall also recognize that −MFE is highly correlated with half-life of mRNA sequences that can be generated given flanking UTRs and an encoded protein, according to DegScore's half-life predictions, as can be seen in the Correlation C column in Table 2 below, where the correlation is between the −MFE of the mRNA and the half-life of the mRNA of contiguous difference enumerations between maximal and minimum MFE as found by CDSFold.

TABLE 2
Correlations between expression level measures of example enumerated contiguous
differences between the minimum and maximal MFE sequences reported by CDSFold.
Correlation A is between difference percentage and −MFE(CDS)). Correlation
B is between −MFE(CDS) and −MFE(mRNA). Correlation C is between −MFE(mRNA)
and HL(mRNA). Correlation D is between difference percentage and HL(mRNA).

| Protein | Correlation A | Correlation B | Correlation C | Correlation D |
|---|---|---|---|---|
| Zm00001eb008690_P001 | 0.987156 | 0.99972 | 0.990774 | 0.982106 |
| Zm00001eb064870_P001 | 0.991002 | 0.999782 | 0.996926 | 0.987761 |
| Zm00001eb064970_P001 | 0.996687 | 0.999947 | 0.984882 | 0.981206 |
| Zm00001eb073650_P001 | 0.99112 | 0.999911 | 0.99595 | 0.991397 |
| Zm00001eb145600_P002 | 0.988229 | 0.999895 | 0.994673 | 0.985527 |
| Zm00001eb145600_P002 | 0.988229 | 0.999895 | 0.994673 | 0.985527 |
| Zm00001eb156910_P001 | 0.993056 | 0.999781 | 0.992719 | 0.990163 |
| Zm00001eb165590_P001 | 0.996267 | 0.999879 | 0.993931 | 0.984804 |
| TolerantALS2 | 0.998079 | 0.999902 | 0.996407 | 0.993483 |
| Zm00001eb183780_P003 | 0.994532 | 0.999697 | 0.993588 | 0.993496 |
| Zm00001eb234360_P001 | 0.994253 | 0.999647 | 0.991194 | 0.990025 |
| TolerantALS1 | 0.997668 | 0.999864 | 0.98995 | 0.988569 |
| Zm00001eb284010_P001 | 0.972205 | 0.999608 | 0.996893 | 0.979618 |
| Zm00001eb299920_P001 | 0.997729 | 0.999792 | 0.995282 | 0.994195 |
| Zm00001eb366090_P001 | 0.993249 | 0.99994 | 0.99741 | 0.989241 |
| Zm00001eb369840_P001 | 0.969149 | 0.999492 | 0.96706 | 0.922587 |
| Zm00001eb369850_P001 | 0.993183 | 0.999265 | 0.993646 | 0.981474 |
| Zm00001eb379110_P001 | 0.987979 | 0.999793 | 0.995634 | 0.976837 |
| Zm00001eb379120_P001 | 0.997234 | 0.999924 | 0.986907 | 0.981707 |
| Zm00001eb379130_P001 | 0.994505 | 0.999837 | 0.988241 | 0.977405 |
| Zm00001eb379140_P001 | 0.985117 | 0.999833 | 0.994611 | 0.979022 |
| Zm00001eb402550_P001 | 0.990078 | 0.999647 | 0.997569 | 0.985904 |
| Zm00001eb402560_P001 | 0.991229 | 0.999946 | 0.996067 | 0.980736 |

While the non-monotonicity of EL adds a degree of complexity to precise design, the level-lowering, enumeration, and/or group testing methods can be used in the narrowing approach to overcome the complexity due to non-monotonicity of EL.

Let (U5,P,U3) denote a triplet of a sequence U5 representing an intended 5′ UTR, a sequence P representing a protein, and a sequence U3 representing an intended 3′ UTR. When useful, an mRNA sequence S can be transformed into a triplet (U5,P,U3) when the coding region sequence of S is known or the UTR sequences of S are known. Let us denote separation of sequence S representing mRNA into triplet form by (UTR5(S),TRANSLATE(CR(S)),UTR(3))=TRIPLET(S).

One skilled in the art shall understand that the coding region sequence of a given mRNA sequence can be determined in non-degenerative situations when the sequence P is available, by producing three digital translations (with first codon starting at indices 1, 2, and 3, respectively) of the mRNA sequence and identifying the translation that contains sequence P. Also, when the untranslated region sequences of S are known, the coding region sequence is the portion of S between the subsequences that extend to either end of S and match the untranslated regions.

We also note that it is also possible to define a triplet (Null, TRANSLATE(S),Null) such that the first and/or third elements are nulls that indicate the respective untranslated regions are not relevant or are not of interest, and the second element is a sequence representing the digital translation of a polynucleotide sequence S to polypeptide encoding.

Then, let us use H((U5,P,U3),t,C) to denote a fluxual gene training approach that accepts a triplet consisting of a 5′ UTR sequence U5, a 3′ UTR sequence U3, and a protein sequence P, as well as a target expression level t, and solution constraint set C. Let H(S,t,C) denote a fluxual gene training approach that accepts an mRNA or coding region sequence S.

In the narrowing approach, iterations of H are used with successively different values of t until a stopping condition is reached. The example narrowing approaches are parameterizable to allow one to retain or prioritize different properties of the result, depending on the need. In some embodiments, the narrowing approach is a level-lowering approach that in some ways resembles (and/or may be implemented as) a binary search with memoization of two or more sequences and their expression window. In some embodiments, the narrowing approach can be viewed as an enumeration of subsets of monotonically changing size with memoization of two or more sequences with closest encountered expression level.

In the context of the narrowing approach, let an expression interval be defined by two values, that includes the intermediate values between the two defining values, and is optionally inclusive or exclusive of one or both of the two defining values themselves. The values defining an interval may be maximum and minimum values with respect to what is of interest for a particular problem or context and what is of interest may differ between specific opportunities for application and are not necessarily required to be the extrema that are known nor the extrema that may be possible.

An expression window is an expression interval that guarantees there exists a sequence available within the interval. This approach makes use of expression windows to identify sequences and uses expression window narrowing to identify sequences that meet or nearly meet specified expression level criteria. Expression window narrowing is a process of reducing the span of the interval of which the expression window is defined.

Expression window narrowing uses the difference between the sequences representing coding regions of RNA to meet or nearly meet specified expression level criteria. When t is within the initial expression window, expression window narrowing results in a sequence that meets the specified expression level estimate criteria or two sequence options to choose from, one option with expression level estimate greater than or equal to the specified expression level criteria and one option with expression level estimate less than or equal to the specified expression level criteria.

Now, we say an expression window [xᵣ,xₛ] is narrower than expression window [xᵢ,xⱼ] when xᵢ≤xᵣ≤xₛ≤xⱼ and either xᵢ<xᵣ or xₛ<xⱼ. An example of the narrowing approach to find monotonically narrower expression windows using one or more guesses is described below. We refer to the example approach as NARROWING. NARROWING can be parameterized by a context initializer, an approach to sequence-guessing, a sequence property checker, a memoization approach, and a termination condition. Different parameter choices can be made that affect the speed to and span of a result that meets or approximates the target expression level.

Let C.sequence represent sequence constraints. Let C represent all constraints.

Then an example enumerative approach denoted H_{CONSTRAIN} using H_{DB} subject to C is as follows:

The following example enumerative approach uses preceding approaches to generate a sequence with expression estimate that may be nearer level t, where t is in the units of an expression level estimator representing approximately the desired expression level (e.g. a percentage, an −MFE, an adjusted −MFE, a half-life). In this example embodiment, the approach does inductive checks for satisfaction of conformation or other mobility constraints with each generated sequence, does allow for sequence constraints, and starts with an existing mRNA sequence or coding region sequence that satisfies C.sequence and C.mobility. When satisfaction of the constraints, C, indicates whether a generated sequence is mobility-analogous to S, for example in cases where secondary structures of sequence untranslated regions define mobility, then H_{CONSTRAIN} is capable of producing sequences mobility-analogous to S.

Let C.sequence.constrain(i,x) denote the addition of a sequence constraint such that a generated sequence must have x at position i.

```
H_{CONSTRAIN}(S,t,C): //enumerative for property constraints
  Rₚᵣₑᵥ = R_{bestlo} = R_{besthi} = S
  Delta_{bestlo} = Delta_{besthi} = Inf
  tₒᵤₜ = EL(Rₚᵣₑᵥ)
  t_{delta} = tₒᵤₜ − t
  i = −1/length(P) * sign(t_{delta})
  For each multiple tᵢₙ of increment i in the expression level range:
    Rₜₑₛₜ = H(S,tᵢₙ,C.sequence)
    tₒᵤₜ = EL(Rₜₑₛₜ)
    Delta = tₒᵤₜ − t
    If Rₜₑₛₜ satisfies C Then:
      If Delta <= 0 Then:
        If |Delta| <= |Delta_{bestlo}| Then:
          R_{bestlo} = Rₜₑₛₜ
          Delta_{bestlo} = Delta
      If Delta >= 0:
        If |Delta| <= |Delta_{besthi}| Then:
          R_{besthi} = Rₜₑₛₜ
          Delta_{besthi} = Delta
    Else:
      D = set of differences between Rₚᵣₑᵥ and Rₜₑₛₜ
      For each d=(i,j) in D:
        C.sequence.constrain(j,Rₚᵣₑᵥ[i])
    Rₚᵣₑᵥ = Rₜₑₛₜ
    Result = (R_{bestlo},R_{besthi})
```

The following enumerative approach uses preceding approaches to generate a sequence with expression that may be nearer level t, where t is in the units of an expression level estimator representing approximately the desired expression level (e.g. a percentage, an −MFE, an adjusted −MFE, a half-life). The approach does not check conformation constraints, but does allow for sequence constraints, uses a protein sequence and flanking UTR sequences as inputs, and does not require an existing polynucleotide coding region sequence to work.

```
H_{PRECISION} ((U5,P,U3),t,C): //enumerative for extra precision
  Rₚᵣₑᵥ = R_{bestlo} = R_{besthi} = Null
  Delta_{bestlo} = Delta_{besthi} = Inf
  i = 1/length(P)
  For each decreasing multiple tᵢₙ of i in the expression level range:
    Rₜₑₛₜ = H((U5,P,U3),tᵢₙ,C.sequence)
    tₒᵤₜ = EL(Rₜₑₛₜ)
    Delta = tₒᵤₜ − t
    If Delta <= 0 Then:
      If |Delta| <= |Delta_{bestlo}| Then:
        R_{bestlo} = Rₜₑₛₜ
        Delta_{bestlo} = Delta
    If Delta >= 0:
      If |Delta| <= |Delta_{besthi}| Then:
        R_{besthi} = Rₜₑₛₜ
        Delta_{besthi} = Delta
  Result = (R_{bestlo},R_{besthi})
```

The following is a level lowering approach that uses preceding approaches to generate a sequence with expression that may be nearer level t, where t is in the units of an expression level estimator representing approximately the desired expression level (e.g. a percentage, an −MFE, an adjusted −MFE, a half-life). The approach does not check conformation constraints, but does allow for sequence constraints, and uses an existing mRNA sequence and/or coding region sequence to work. The approach helps keep the editing on a direct path to reach extrema when each individual difference position of a polynucleotide sequence is edited in series. The approach is denoted H_{MINMOD} using H subject to the sequence constraints and/or required resolution of C.

```
H_{MINMOD}(S,t,C): //for extra precision
  Transform S to (U5,P,U3)
  If EL(S) <= t Then:
    tₕᵢ_in = EL_{MAX}
    PBS = H((U5,P,U3),tₕᵢ_in,C.sequence)
    tₗₒ_in = t
    UBS = S
  Else:
    tₕᵢ_in = t
    PBS = S
    tₗₒ_in = EL_{MIN}
    UBS = H((U5,P,U3),tₗₒ_in,C.sequence)
  Result = H_{LOWERING}(PBS,UBS,tₕᵢ_in,tₗₒ_in,t,C.sequence)
```

The following is a level lowering approach that uses preceding approaches to generate a sequence with expression design level estimate that may be nearer level t, where t is in the units of an expression level estimator representing approximately the desired expression level (e.g. a percentage, an −MFE, an adjusted −MFE, a half-life). The approach does not check conformation constraints and uses an existing mRNA and/or coding region sequence to work. The approach helps reduce the size of the difference between maximal and minimal expression levels by using gene replacement and/or standardizing what is already present, to reduce efforts for when subsequent rounds of inductive gene training are expected to be implemented in DNA. Let us denote H_{REPLACE} using H subject to the sequence constraints of C as follows:

H_{REPLACE}((U5,P,U3),t,C): //for extra precision
tₕᵢ_in = EL_{MAX}
PBS = H((U5,P,U3),tₕᵢ_in,C.sequence)
tₗₒ_in = EL_{MIN}
UBS = H((U5,P,U3),tₗₒ_in,C.sequence)
Result = H_{LOWERING}(PBS,UBS,tₕᵢ_in,tₗₒ_in,t, C.sequence)

Let C.resolution represent the desired nearness of the expression level of the resulting sequence to t. The following is a helper method for the above two level-lowering approaches:

```
H_{LOWERING}(PBS,UBS,tₕᵢ_in,tₗₒ_in,t,C): //for extra precision
  BestPS = PBS
  BestUS = UBS
  PS_{Delta} = PT_{Delta} = | EL(PBS) − t |
  US_{Delta} = UT_{Delta} = | EL(UBS) − t |
  Delta = PT_{Delta}
  While (Delta > C.resolution AND (tₕᵢ_in−tₗₒ_in) > (1/length(PBS)) :
    tᵢₙ = tₕᵢ_in − (tₕᵢ_in − tₗₒ_in) / 2
    Current = H((U5,P,U3),tᵢₙ,C.sequence)
    tₒᵤₜ = EL(Current)
    CT_{Delta} = |t − tₒᵤₜ|
    UC_{Delta} = |EL(UBS) − tₒᵤₜ|
    PC_{Delta} = |EL(PBS) − tₒᵤₜ|
    If (UT_{Delta} >= UC_{Delta}) OR (UT_{Delta} + UC_{Delta} <= CT_{Delta}) Then:
      tₕᵢ_in = tᵢₙ
      If CT_{Delta} <= US_{Delta} Then:
        BestUS = Current
        US_{Delta} = CT_{Delta}
    If (PT_{Delta} >= PC_{Delta}) OR (PT_{Delta} + PC_{Delta} <= CT_{Delta}) Then:
      tₗₒ_in = tᵢₙ
      If CT_{Delta} <= PS_{Delta} Then:
        BestPS = Current
        PS_{Delta} = CT_{Delta}
  Result = (BestUS, BestPS)
```

### Approach 6: Functionally Analogous Gene Training

In some circumstances, it is desirable to replace or edit an existing gene to produce a modified or different polypeptide or protein. Rosetta Fold Diffusion, amino acid perturbation, and assisted continuous evolution are some examples of protein engineering tools that can be used to help design or redesign a protein sequence including one or more polypeptides of interest. Redesigned proteins may be functionally analogous with respect to an existing protein sequence of interest, while designed proteins can be new or functionally analogous with respect to an existing protein sequence of interest.

In a sixth approach to fluxual gene training, we describe how to use other fluxual gene training approaches to enable functionally analogous gene training. Functionally analogous gene training can be used in experimental validations of protein engineering, as well as in processes such as gene replacement and/or gene editing and/or transformation efforts for production crop improvements.

Let sequence P_{T} represent a polypeptide of interest, chosen from a set of existing polypeptide sequences or generated using protein sequence engineering tools. Then, a sequence including or encoding the polypeptide of P_{T}, where P_{T} is functionally analogous to a polypeptide P_{S}, and where P_{S} is encoded by a second sequence, S representing an mRNA, is trained to produce a third sequence, R representing an mRNA encoding P_{T}, wherein EL(R)≈EL(S), where EL is an estimator of expression level including for examples, a percentage, an −MFE, an adjusted −MFE, a half-life.

In some embodiments where the sequence provided represents P_{T}, a method of reverse translation is used to produce a coding region sequence that is flanked by the untranslated region sequences of S, which together we refer to as sequence T representing an mRNA to be used as input for fluxual gene training. In some embodiments, the sequence provided represents an mRNA encoding of a protein including P_{T} with coding region identified explicitly, for example provided in components, for example as a triplet, and a concatenation of its components is composed as a sequence T representing an mRNA to be used as input for fluxual gene training. In some embodiments, the sequence provided represents an mRNA encoding of a protein including P_{T} with coding region identified implicitly, for example where untranslated regions are identified by constraints described in C, and a solution using a method (including ones disclosed here) is composed or generated as an mRNA T to be used as input for fluxual gene training. In some embodiments, the sequence provided represents an mRNA encoding of a protein including P_{T} with coding region specified using a hybrid approach where some sequence elements are generated or defined implicitly and some sequence elements are specified explicitly, and a solution using a method (including ones disclosed here) is composed or generated as an mRNA T to be used as input for fluxual gene training.

Then, examples that allow for generation of R from S and T can be done as follows, where H* represents an in silico approach to aiming gene expression design level, for example including but not limited to aforementioned gene training narrowing approaches, and EL is an approach (e.g. in silico) for assessing gene expression.

H_{ANALOGOUS}(S,T,EL,C)
Result = R = H*(T,EL(S),C)

Similarly the following is an example embodiment where untranslated regions of S may be encoded in the constraints, C.

H_{ANALOGOUS}(S,P_{T},EL,C)
Result = R = H*(P_{T},EL(S),C)

### Implementation in a Genome

To implement the result of fluxual gene training in a genome, the first step is to determine whether an existing gene site will be edited or whether a new gene site will be created. New gene sites can be created through genome engineering tools. When an existing gene site is edited, one or more edits to one or more parts of a gene are made in a genome to change the gene, or one or more sections of DNA are replaced to replace one or more existing portions of DNA representing one or more genes, for example via multiplexing. In gene replacement introns might be desirable to retain in polynucleotide designs when the introns serve a purpose such as but not limited to mobility, but introns may also not be retained in polynucleotide designs if the introns are known to have no function, or if the risks of attempted intron inclusion outweigh the risks without intron.

In the case of gene training in a genome, identification of the coding region sequences of the original DNA sequence may assist with specification of the necessary gene modifications. When sections of DNA (or primary transcript) sequence that represent exon (or coding region) sequences are already identified or available, and the splicing positions can be understood to remain unchanged following gene modification, then an approach such as and including the one that follows can be used to describe the specification for a modified DNA (or primary transcript) that includes existing intron sequences while making exonic (or coding region) sequence substitutions.

Given a sequence U, let U(i,j) denote the sequence U[i], U[i+1], . . . , U[j−1], j>i. We say a sequence of pairs (i₁,j₁), i₂,j₂), . . . , (iₙ,jₙ) are encoding when iₖ<jₖ<iₖ₊₁<jₖ₊₁ for all k, 1<=k<n. Let P=(i₁,j₁), (i₂,j₂), . . . , (iₙ,jₙ) be an encoding sequence of pairs and let Pₖ=Pₖ₋₁+jₖ₋₁−iₖ₋₁ for 1<k<=n and P1=1.

Let X(U,P) denote the sequence U(i₁,j₁), U(i₂,j₂), . . . , U(iₙ,jₙ).

Let I(U,P) denote the sequence U(j₁,i₂), U(j₂,i₃), . . . , U(jₙ₋₁,iₙ).

We say an encoding sequence of pairs R is the composition of P when R=(P₁,P₂), (P₂,P₃), . . . , (Pₙ,Pₙ₊₁).

When P is an exonic splicing (or coding region portions thereof) of gene U, then X(U,P) and I(U,P) are the sequence of exon sequences (or coding region sequences) and intron sequences of U, respectively and the concatenation of the sequences of sequence X(U,P) is the mRNA (or coding region sequence), S, spliced according to P. And when T=H*(S,t,C), then the new gene sequence V can be described as the concatenation of X(T,R)₁,I(U,P)₁,X(T,R)₂,I(U,P)₂, . . . , X(T,R)ₙ, which we refer to as the construction from P, U, and T.

Then, the difference between U and V according to an alignment in the context of the broader genome can help identify changes that need to be made to produce G_{V} from the original genome G_{U}, wherein the identified DNA changes can be used to produce a sequence of edits or replacements t₁,t₂, . . . , tₖ such that tₖ (t . . . (t₂ (t₁ (G_{U}))))=G_{V} wherein each edit or replacement may be designed to resolve one or more changes of one or more genes.

For example, let (i,j) be an element of the difference between G_{U} and G_{V} (e.g. implied by the difference between U and V) according to an alignment. Then, a transformation that addresses an individual needed change (i,j) can be described as an edit of U[i] to V[j] at position i in genome G_{U} to produce G_{U}′. Then a next individual transformation that addresses needed change (i′,j′) from difference between G_{U}′ and G_{V} can be described as an edit of U′ [i] to V[j] at position i′ in genome G_{U}′ to produce G_{U}″, and so on. Similarly unaligned indices of U and V help identify needed deletions and additions in the broader context of transforming G_{U} to G_{V}, wherein deletions and additions may also similarly be applied until G_{V} is produced.

Each transformation is to be implemented with a genome engineering process such as including but not limited to gene editing done via CRISPR [U.S. Pat. No. 8,697,359 B1], gene replacement done via RMCE [Schlake et al., Turan et al.], or other transformation technology, wherein each edit or replacement may be designed to address more than one need by performing more than one edit or replacement together. For example, more complex edits or replacements can be performed, for example wherein one or more difference, deletion, and/or addition may be encapsulated in one or more edits or replacements, depending on the availability and cost of genome engineering tools and/or components thereof, such as enzymes and PAM sequences, or by using multiplexing or a flexible editing enzyme or apparatus that allows for polynucleotide edits.

When the exonic region coordinates of DNA sequences or primary transcript sequences that represent exons or coding region are not already identified or available, the first step is to identify the exonic region coordinates for the mRNA and/or coding region of interest. Various approaches for exonic and/or coding region coordinate identification exist [Kapustin et al], some of which rely on knowledge of the mRNA sequence or coding region sequence [Jammali et al].

The following is a disclosure of a new method that identifies accurate exonic region coordinates and/or coding region coordinates P from two sequences, S and U, where S represents an mRNA or coding region, and U represents the primary transcript and/or DNA strand from which S originated, when an accurate splicing is a unique splicing that has the minimum number of exons, over all possible splices, excepting when the concatenation of accurate exonic regions of U, X(U,P), are unequal to S.

Section Reporting

```
m = length(S)
n = length(U)
Let LCP_LR(_{j,i}) be a function that returns the longest common prefix
between strings Sᵢ,Sᵢ₊₁,...,Sₘ and
Uⱼ,Uⱼ₊₁,...,Uₙ as described by [Fischer et al., Kasai et al., Karkkainen et
al.]
Let X be such that X[i] = infinity if 0<i<m+2
Let M be such that M[i] = 0 if 0<i<m+2
Let I be such that I[i] = −1 if 1<i<m+2 and I[i] = 0 if i=1
Let J be such that J[i] = −1 if 1<i<m+2 and J[i] = 0 if i=1
For each i from 1 to m:
  For each j from max(1,J[i])+M[i] to n:
    start = X[i]
    lcp = LCP_LR(j,i)
    next = i+lcp
    best = X[next]
    If start is not infinity AND start + 1 < best Then:
      X[next] = start + 1
      M[next] = lcp
      I[next] = i
      J[next] = j
If I[m+1] equal −1 Then:
  Result = None // There was no solution.
Else:
  Let i = m+1
  While i>1:
    stack.push((J[i], J[i]+M[i]))
    i = i − M[i]
  Result = pop stack into result
```

Applied to example sequences S=ACTTUGTAC and U=ACTACTTGTACTUGTUAC, the method gives the following result: [1,4), [12,16), [17,19), which can be visualized as ACTACTTGTACTUGTUAC, wherein the bolded polynucleotides represent the order-preserving alignment between S and U.

One may determine whether the result P is a unique solution by applying the method to the reverse of S and reverse of U, and transforming the resulting encoding sequence of pairs (i₁,j₁), (i₂,j₂), . . . , (iₙ,jₙ) to P′=(length (U)-jₙ+1,length (U)-iₙ+1), (length (U)-jₙ₋₁+1,length (U)-iₙ₋₁+1), . . . , (length (U)-j₁₊₁,length (U)-i₁₊₁), and comparing P to P′. If there are no differences between P and P′, then P is a unique solution.

Applied to the above example sequences, we have [4,7), [12,16), [17,19), which can be visualized as ACTACTTGTACTUGTUAC, wherein the bolded polynucleotides represent the order-preserving alignment between the reverse of S and the reverse of U.

If P is not a unique solution, we adjust P with U using the following heuristic to recognize one or more intron start sequences and one or more intron end sequences. In the below example the selected intron start sequences are GU(and GT in DNA) and the selected intron end sequence is AG.

```
Let IS = {“GU”,”GT”}
Let IE = {“AG”}
For each x from 2 to n:
  Done = False
  While Not Done and U[jₓ₋₁−1] equals U[iₓ−1]:
    Left= U(jₓ₋₁,jₓ₋₁+1)
    Right= U(iₓ−2,iₓ−1)
    If Left in IS and Right in IE Then:
      Done = True
    Else:
      jₓ₋₁ = jₓ₋₁ − 1
      iₓ = iₓ − 1
```

We refer to P as the likely splicing of U with respect to S if the adjustments of P and P′ are equal. If the adjustments of P and P′ are still not equal, then one might apply a heuristic that selects one of the two solutions with minimum span (i.e. minimum jₙ−i₁) with caution. However, our experience is this is ultra rare in practice.

In the case of replacing a gene in a genome, a gene with sequence U may be replaced by a nucleic acid sequence V or X(V,P), using, for example, a site-directed recombinase mediated cassette exchange [Bode et al], to replace the gene.

One skilled in the art will recognize that one or more embodiments of gene training can be applied to optimize the expression of one or a plurality of genes, alone or in combination, for experimentation, product development, and/or simulation, including but not limited to systems biology modeling, protoplast experimentation, and/or other computational or empirical methods, and that the partial difference need not be contiguous to practice the methods of the disclosure.

### Agricultural Applications and Pipelines

### Improving Traditional Signal Analyses

Approaches to traditional signal analyses, genomic selection, and genomic change may be improved by using the methods of fluxual gene training. Existing SNP-based genomic signal analyses map SNPs to {0,1,2} or {−1,0,1} states for statistical analyses where each SNP is a polymorphism between transcripts. This disclosure of a transcript-based half-life gradient from −100% to 100% or 0% to 100% and/or normalization and/or regularization thereof can be applied in transcript-inclusive signal analyses.

In one or more embodiments, one converts transcript sequence −MFE and/or half-lives and/or other expression level related estimates (and/or in some circumstances even difference percentages between extremal sequences) to percentages between [0%, 100%] or [−100%, 100%] where each percentage represents the degree between extremal expression level estimates for the transcripts encoding one or more proteins. Then each transcript percentage (or homologous analogous transcript percentages, or average or sum thereof) may become a variable in one or more analyses including but not limited to QTL and GWAS analyses. By using a gradient for each analogous transcript, one may reduce the number of variables and may improve predictive and prescriptive outcomes.

Furthermore, the expression design levels of analogous and non-analogous proteins may be viewed, compared, and evaluated using difference percentages, negative length-adjusted MFE, and/or half-life of the proteins in visual analyses and tools therefore, as well as in other analyses, such as systems biology type simulations, for example to tune the expression design levels of mRNAs in a pathway to tune enzyme production and consequent reaction rates.

To practice the invention, one includes two terms per protein, the first term representing the effect of the protein analog, the second term representing the effect of the expression level.

### Group Testing of Genomic Signals

In most circumstances, the results of signal analyses are a region or set of genes that are putatively important. However, successful exploitation of such found regions and/or genes is often elusive. Fluxual gene training provides novel opportunities to understand and exploit results of signal analyses.

For example, given a set of transcripts resulting from signal analysis (e.g. GxP, GxE, and others), apply one or more types of group testing to determine a minimal subset of transcripts for which it is necessary to (optionally modestly) increase (or decrease) half-life to see phenotypic improvement. This differs from the approach used in current state-of-the-art signal analyses that leverage only knock out, knock down, or copy adding approaches.

### Introduction of New Variation

Breeding programs and biotech programs have thus far been limited to existing germplasm and existing natural variation found across the globe. Furthermore, signal analyses intended to isolate exploitable causal genetic variation are often confounded by promoter variation, UTR variation, amino acid sequence variation, and variations of hundreds or thousands of other genes. Fluxual gene training can assist with overcoming limitations of signal analyses by allowing for the creation of precise and controlled variation. For example, given a genome G, one may create many genome variants G₁,G₂, . . . . Gₖ each with one or more transcript variants, each with one or more half-lives. Then, fluxual gene training is compatible with both new and existing crop improvement techniques.

For example, ideally working together, those with understanding of the biology might apply existing knowledge to identify opportunities for crop improvement, those who have an understanding of computational techniques might apply such techniques to identify opportunities for crop improvements, and those with knowledge of breeding and germplasm might leverage breeding techniques to interbreed or recombine with other germplasm.

### Directional Modification of Half-Life of One or More Specified Genes

In some circumstances, getting the desired phenotype may be reduced to a directional change in expression with experimentation and repetition as necessary or desired. For example, decreasing plant stature or increasing hormone production, without a priori known target half-life, can be done by generating phenotypes A with specified transcript half-life HL_{A} and then designing transcript with increased (or decreased) half-life HL_{B} to be greater than (or less than) HL_{A} to produce phenotype B with greater (or lesser) expression of polypeptide translated from specified transcript mRNAs.

### Aiming Half-Life of One or More Specified Genes

However, in other circumstances, for example with an a priori known target half-life or relative expression design level, one can specify the half-life or difference percentage as t in one or more approaches to gene training. For example, decreasing plant stature or increasing hormone production, without a priori known target half-life, can be done by generating phenotypes A and B with targeted transcript half-lives HL_{A} and HL_{B}, and then using lowering to design transcript half-life HL_{C} to be between HL_{A} and HL_{B} to produce a phenotype between A and B.

### Pipeline for Agriculture

TABLE 3
Gene Training for Application to Agriculture and Carbon Capture

| | |
|---|---|
| a) | Conduct genetic/transcriptomic signal analysis to identify genes/variants/promoters of interest |
| | (e.g. via simulation, experimentation, solver, deep learning, artificial intelligence). |
| b) | Determine expression window(s) of interest (optionally identifying current expression level). |
| c) | Specify expression design level, an expression design level t, for each gene of interest (e.g. for |
| | evaluation or improvement). |
| d) | Implement gene expression design levels with traditional or modern techniques (e.g. editing, |
| | RMCE). |
| e) | Repeat a-d as desired (e.g. assess new genomes/transcriptomes for performance, pick features, |
| | precisely modulate expression, refine genomes/transcriptomes). |
| f) | Optionally repeat a-e as desired from different starting points/genomes/transcriptomes. |

### Antisense Gene Training

Antisense gene training is focused on the computer-aided design of maximally-specific antisense oligonucleotides (MS-ASOs). Traditional antisense oligonucleotides (ASOs) design is a process that results in the identification of a transcript of which complementary nucleic acids are to be designed [U.S. Pat. No. 5,166,195]. However, the process of designing complementary nucleic acids with the desired properties remains challenging because application of the current state-of-the-art methods start with discovery of a nucleic acid sequence and wherein the problem is reduced to finding a complementary sequence within the prescribed range of lengths that does not observably negatively affect the host.

Traditionally designed ASOs may have off-target effects. When the ASO is too short, more than one gene may be affected. When the ASO is too long, the ASO may affect a non-pathological variant necessary for healthy living. Also, some ASOs may bind to unintended targets because of multiple possible pairings. In this disclosure, we describe a computer-assisted method of design of polynucleotide sequences for maximally-specific antisense oligonucleotides to comprise that are neither unnecessarily short, nor unnecessarily long, and respect nucleotide pairings, given a pathogenic transcript. The method is to assist in the development of ASOs, subject to other knowledge, for example guidance about the accessibility of the region in the target pathogenic RNA.

### Pairing Sensitivity and Off-Target Mitigation

Let us define the notion of a pairing-sensitive reverse complement (PSRC) of an ASO to be a regular expression that describes the set of polynucleotides of same length as the ASO to which the ASO may completely pair.

For example, a PSRC that allows for Watson-Crick base pairing complement for DNA is shown below:

PSRC(A)=T
PSRC(C)=G
PSRC(G)=C
PSRC(T)=A

An example PSRC that allows for an additional pairing of U and G, and A and U instead of A and T for RNA is shown below:

PSRC(A)=U (matching uracil)
PSRC(C)=G
PSRC(G)=[CU] (matching cytosine or uracil)
PSRC(U)=[AG] (matching adenine or guanine)
PSRC(T)=A

An example combined PSRC that allows all the above for convenience is shown below:

PSRC(A)=[UT] (matching uracil and thymine)
PSRC(C)=G
PSRC(G)=[CU] (matching cytosine or uracil)
PSRC(T)=A
PSRC(U)=[AG] (matching adenine or guanine)

However, one skilled in the art shall recognize that the PSRC may be extended or be refined to describe pairings of nucleotides, alternative nucleotides, or analogous nucleotides. The PSRC should be chosen to be a digital twin of the biochemistry of pairing by nucleotides, alternative nucleotides, and analogous nucleotides.

A pairing-sensitive reverse complement may differ from the traditional reverse complement in that the PSRC of a nucleic acid may match more than one nucleic acid. For example, in the above example PSRC, the PSRC of guanine (G) is a regular expression [CU], matching cytosine or uracil with respect to RNA. The specific definition of the PSRC can be refined to suit the need, for example by adding nucleotides (or analogs thereof) and/or pairing rules.

The pairing-sensitive reverse complement (PSRC) of a sequence S=s₁,s₂, . . . , sₖ₋₁,sₖ is the regular expression defined by the sequence PSRC(S)=PSRC(s_{K}),PSRC(sₖ₋₁), . . . , PSRC(s₂),PSRC(s₁). The pairing-sensitive reverse complement of a sequence may differ from the traditional nucleic acid reverse complement in that the PSRC of a sequence is a regular expression that accepts a sequence if it may pair with the original sequence when the original sequence is an antisense oligonucleotide (ASO).

Given a target RNA sequence and a reference set of polynucleotide sequences, an antisense oligonucleotide (ASO) sequence is target-specific for the RNA sequence with respect to the reference set if the PSRC regular expression of the ASO matches a subsequence of the target sequence but no other sequence of the reference set. For example, if a reference set represents an individual's transcriptome, then the number of distinct sequences identified by probe-based sequencing of each of the strings matching the PSRC regular expression must total one (1) to be considered target-specific.

An ASO is a maximally-specific antisense oligonucleotide (MS-ASO) with respect to the target sequence and reference set if no proper prefix of the ASO sequence is target-specific and no proper suffix of the ASO sequence is target-specific to the target sequence with respect to the reference set.

A maximally-specific ASO minimizes the off-target problem when a corresponding supplemental polynucleotide is not used, by minimizing pairings with non-variant polynucleotide positions, while ensuring specificity to a target RNA when the target RNA is transcribed from an undesired gene variant and the reference set is the set of sequences of an intended recipient's transcriptome, and optionally the sequences of the intended recipient's genome.

### Minimal Signatures and Context Sensitivity

Let us define the notion of a context-sensitive regular expression expansion (CSREE) of a nucleotide to be a regular expression to represent the set of possible nucleotides that complement its complement, which is dependent on what pairings are possible, which is context dependent (for example which may be the case when designing an ASO versus designing a marker). The context-sensitive regular expression expansion (CSREE) of a polynucleotide sequence S=s₁,s₂, . . . , sₖ₋₁,sₖ is a regular expression defined by the sequence CSREE(S)=CSREE(s₁),CSREE(s₂), . . . , CSREE(sₖ₋₁), CSREE(sₖ). A CSREE may be useful in generating minimal signatures and sequences for antisense oligonucleotides.

We refer to the expansion as the identity or supplemental when the CSREE of each relevant nucleic acid is equal to itself. We refer to the CSREE as pairing-sensitive when it is defined with respect to the PSRC. Please note that an identity CSREE is a special case of a pairing-sensitive CSREE. Using the above example PSRCs defined for convenience, associated pairing-sensitive CSREEs can be defined as follows.

CSREE(A)=A or CSREE(A)=[AG] (matching adenine or guanine)
CSREE(C)=[CU] (matching cytosine or uracil)
CSREE(G)-G or CSREE(G)=[AG] (matching adenine or guanine)
CSREE(T)=T
CSREE(U)=[UT] (matching uracil or thymine) or CSREE(U)=[CU] (matching cytosine or uracil)

Here it is noted that given nucleotides sᵢ and sⱼ of a sequence S, where sᵢ=sⱼ, CSREE(sᵢ) need not be equal to CSREE(sⱼ). For example, when sᵢ=sⱼ=A, it is possible that CSREE(sᵢ)=A and CSREE(sᵢ)=[AG]. In addition, it may be helpful that when given a target-sequence of given length, one also defines the target-sequence CSREE of same length, whereby we say the CSREE associated with a subsequence of the target-sequence is defined by the range [i,j] of the target-sequence CSREE when the subsequence of the target-sequence has range [i,j]. For convenience and for the purposes of this disclosure, we use the terminology CSREE of the (sub) sequence to be understood to mean the CSREE defined by the range [i,j] of the target-sequence CSREE wherein the sequence is the subsequence of the target-sequence defined by the range [i,j].

Then, with an understanding that molecules of a single ASO design may pair with regions of one or more polynucleotides wherein the sequences of the paired regions differ, we define a signature to be a subsequence of a given target-sequence wherein the CSREE of the subsequence matches within the target-sequence but does not match any other sequence of the reference set. For the remainder of this disclosure, a “signature” shall be understood to be with respect to the context of an applicable and relevant target-sequence, target-sequence CSREE, and reference set. Biochemistry and digital approaches may be used to determine whether a sequence is a signature. Biochemistry approaches can involve identifying the distinct number of sequences identified by a set of probe-based sequencing assays of a transcriptome and optionally a genome. Digital approaches may include regular expression pattern matching, string search(es), or lookup(s) in a reference set of sequences or an index thereof can be used to determine whether a sequence is a signature.

With the digital approach, we say that a signature is pairing-sensitive when the signature is found with a pairing-sensitive CSREE. With a probe-based sequencing biochemistry approach, we say a signature is found when the number of distinct sequences identified in total by the set of probe-based sequencing assays for the strings exactly matched by the pairing-sensitive CSREE is one (1). We say that a signature is supplemental when the signature is found with a supplemental CSREE.

Then we refer to a signature as right-minimal when the signature does not have a proper prefix that is also a signature with respect to the CSREE. Similarly, we refer to a signature as left-minimal when the signature does not have a proper suffix that is also a signature with respect to the CSREE. We refer to a signature as doubly-minimal when the signature is both left-minimal and right-minimal.

One skilled in the art shall recognize there are multiple and substantially equivalent approaches to find a minimal signature, for example including but not limited to, probe-based sequencing for the individual sequences represented by a CSREE, searching for individual sequences represented by a CSREE, searching for their reverse complements, using regular expression pattern matching in a sequence, a graph, or an index thereof.

One skilled in the art shall recognize variants of, deterministic finite automata (DFA) of, and/or decorated versions of suffix arrays, suffix trees, their extensions, generalized versions, and augmentations such as data structures for longest common prefix (LCP) queries, inverted suffix arrays, can assist with the regular expression search, for example by intersection of a suffix DFA with a regular expression DFA, matrix multiplication, graph traversals, and/or even substring search can suffice when the search includes all the sequences represented by the regular expression.

Here we present digital and biochemistry approaches to identify signatures. The digital approaches uses a pathological sequence to search for the subsequences in the transcriptome of the intended treatment recipient and optionally genome. The biochemistry approaches uses a pathological sequence together with ability to perform biochemical probing or probe-based sequencing the transcriptome and or genome of the intended treatment recipient. Then given an approach to determine if a sequence is a signature, the additional techniques may be used to identify one or more minimal signatures.

### Approach 1: a Suffix Automata-Based Digital Approach for Signature Identification and Design:

In one example approach, one may produce a suffix tree and suffix links from a suffix array and its LCP array and use the suffix tree to check successively larger prefixes of a query string until the substring of the query does not exist in the suffix tree more than the number of times it appears in the query string (as can be determined with the LCP array), at which point the string is a right minimal signature and then use suffix links to consider suffixes in order of decreasing length to test for double-minimality, for example by using approaches 3 and/or 4 below.

### Approach 2: a Matrix-Based Digital Approach for Signature Identification and Design

In a second example approach, a sparse matrix M can be defined as the graph of allowable nucleotide sequences (where each edge is labeled with a nucleotide). In some embodiments, subgraph representations of each amino acid can be connected to represent coding regions. Transitions can be added between adjacent exons according to one or more splicings or to help represent so-called indel variants, for example using null transitions and their transitivity. M can then be used to represent a reference set.

Then the sparse matrix can be separated into a set of matrices, (e.g. one matrix per nucleotide A,C,G,T,U, and 0, where 0 represents transitive closure of null transitions, would be 6 matrices M[A],M[C],M[G],M[T],M[U],M[0] respectively), where each matrix retains the exact set of nodes from M, but matrix for nucleotide b retains exactly the set of edges of M labeled b (with each edge represented as a one in the sparse matrix).

Then, a search for existence of a polynucleotide sequence can be represented as a series of matrix vector multiplications and/or graph/matrix unions together with a final count of nonzero elements in the resulting vector. For example, a search for the string ATTACCA comprises the multiplication SEARCH (ATTACCA,ONES)=M[A]*(M[0](M[C]*(M[0](M[C]*(M[0](M[A]*(M[0](M[T]*(M[0](M[T]*(M[0](M[A]*ONES)))))), wherein ONES represents a vector of ones with same number of nodes as M, and M[0](V)=M[0]*V+V. An example matrix multiplication technique of determining whether a sequence is a signature is as follows. For example, determining whether ATTACCA is a signature further comprises computing the number of matching paths by further multiplications, using the matrix of the graph with edge direction reversed as follows, where REVERSE(S) indicates the string S in reversed order: SEARCH(REVERSE(S),MIN(SEARCH(S,ONES),1)), wherein MIN sets the nonzero elements of the vector to one (1). Then, the sum of the resulting nodes (identified by non-zero values in resultant vector) gives the number of paths that have the path-label of the query string S. If the number of paths in the matrix matches the number of paths in the query string, then the query string is a signature.

A similar process can be applied if searching for a regular expression an extra matrix per bracketed nucleotide set is created for each combination of bracketed nucleotide. For example, searching for A[TU]TA[CU][CG][A] can be done with additional matrices M[TU], M[CU], and M[CG] such that an edge is in matrix M[b1b2] if and only if either the edge is in M[b₁] or M[b₂], where b₁ and b₂ are each a nucleotide, carried out with multiplication M[A]*(M[0](M[CG]*(M[0](M[CU]*(M[0](M[A]*(M[0](M[T]*(M[0](M[TU]*(M[0](M[A]*ONES)))))), again with the sum of the elements of the vector produced by SEARCH(REVERSE(S),MIN(SEARCH(S,ONES),1)) indicating whether the query string is a signature, and if there does not exist a prefix of the query regular expression that is also a signature, then the signature is right minimal, and if there does not exist a suffix of the query regular expression that is also a signature, then the signature is left minimal.

The multiplications themselves may be accelerated using specialized hardware including, but not limited to, one or more GPUs. Enumerating minimal signatures of a target sequence can also be accelerated, for example using matrix multiplication memoization tricks.

### Approach 3: An Approach to Find a Doubly-Minimal Signature

Here we now describe three approaches to identifying a single doubly-minimal signature in a set of sequences (either biological or digital) with respect to a target-sequence CSREE, if a doubly-minimal signature can be ascertained. Which of the three approaches recommended for use depends upon what information is already known that can be used as a hint. We refer to the three approaches as the signature-seeded approach, the site-seeded approach, and the no-seed approach. Each of the three approaches can be practiced on a genome or a transcriptome or the sequences thereof. These three approaches are described below.

The signature-seeded approach to identifying a doubly-minimal signature assumes that we already have a known signature that satisfies optional constraints and may or may not be a minimal signature. The site-seeded approach assumes that we know of a path (or range or site of variation) within the target sequence that must be at least partially spanned to be a signature of interest, if one exists. The no-seed approach does not require additional information beyond the target-sequence, target-sequence CSREE, and reference set.

When a doubly-minimal signature is to be determined via biochemical probing of a transcriptome (as opposed to via computation on the sequences of a transcriptome or probe-based sequencing) then constraints may apply or be helpful. For example, choosing an exon-exon junction may be helpful with some biochemical probe chemistries and a maximal path length may be a practical limitation of the biochemical probe chemistry for reliable reporting.

### Signature-Seeded Approach:

With the signature-seeded approach, prefixes of the known signature are considered with a (not necessarily balanced) set of searches, for example a binary search keyed on prefix length or iteratively by decreasing length order until the shortest prefix that is also a signature is found, which must be a right minimal signature.

Then suffixes of the known right minimal signature are considered with a set of searches, for example one or more binary searches keyed on prefix length or iteratively by decreasing length order until the shortest suffix that is also a signature is found, which must be a doubly minimal signature.

If there is a site-spanning constraint, an alternative approach to finding a minimal signature is to consider a prefix of a signature if there are more prefixes of the signature that span the site than suffixes of the signature that span the site, and consider a suffix of the signature otherwise until a left or right minimal signature is found, then once a left-minimal or right-minimal signature is found one further considers the prefixes and suffixes, respectfully, of the minimal signature in decreasing order to find a doubly minimal signature.

### No-Seed Approach:

With the no-seed approach to identifying a doubly minimal signature, the prefixes of the target sequence may be considered with a set of (not necessarily balanced) searches, for example one or more binary searches keyed on prefix length or iteratively by increasing length order until a signature is identified, until the path is of maximal length like when the end of the target sequence is reached, or the target sequence is determined to not be a signature. If a signature is identified, the signature is used together with the signature-seeded approach to identify a doubly minimal signature.

### Site-Seeded Approach:

With the site-seeded approach, a path is first chosen that intersects with the known site. If the chosen path gives a signature in the target sequence, the signature-seeded approach can be used to find a doubly-minimal signature. If the chosen path does not give a signature in the target sequence, the path is extended in either or both directions until the path identifies a signature (and then the signature-seeded approach is applied).

In the case an additional constraint is necessary on the length of the path, for example because of length-limitations on reliability of probes for biological sequences, and the maximal path length is reached without identifying a signature, then one may identify the set of paths with maximal path length that span the known site, and check whether each one until a signature is found (after which the signature-seeded approach is applied), or until it is determined that no signature satisfies the constraints.

### Approach 4: Enumerating all the Remaining Doubly-Minimal Signatures

Given a doubly-minimal signature in a target sequence, the following example procedure can be used to identify all the remaining minimal signatures with respect to the given target-sequence CSREE.

### Example Procedure

Given an initial state identifies a sequence that is a left minimal signature, the right extensions of the initial state can be considered in order of increasing length to also identify left minimal signatures, until we identify an extension [i,j] of the initial state whose longest proper suffix is a right minimal signature (or j has reached the end of the target sequence, in which case the procedure is terminated). We define the intermediate state of an instance of the procedure as the range [i+1,j].

Given the sequence described by the intermediate state is a right minimal signature, the suffixes of the intermediate state are considered in order of decreasing length to also be right minimal signatures, until we identify a suffix of the intermediate state that is also a left minimal signature, at which point we consider the instance of the procedure to be finished. We define the ending state of an instance of the procedure as the range that gives the doubly minimal signature and ends at the same endpoint of the intermediate state.

One skilled in the art shall recognize that the endpoints of the ranges representing states of the procedure increase monotonically, and that given the procedure, it is easy to construct a substantially equivalent procedure that monotonically decreases the endpoints of the ranges representing the states. We refer to the procedure that monotonically increases the endpoints of the ranges representing the states as the right-reporting procedure. We refer to the procedure that monotonically decreases the endpoints of the ranges representing the states as the left-reporting procedure.

Furthermore, given a doubly-minimal signature in a target sequence, the ending state of one execution of the procedure can be used as the initial state of another execution of the same procedure, and that when applied in this manner each iteration of the right-reporting or left-reporting procedure identifies at least one additional minimal signature of each type with range endpoints that monotonically increase or decrease, respectively (if an additional minimal signature exists).

The procedures for finding a single doubly-minimal signature, together with the left-reporting procedure and/or right-reporting procedure can be used to enumerate all doubly-minimal signatures of the target-sequence with respect to the reference set and target-sequence CSREE.

To do this, one first uses the procedure to find a single doubly-minimal signature (if one exists). Then given the doubly minimal signature, and with respect to the CSREE, all doubly-minimal signatures can be reported from the union of the results of iteration (until termination) of the right-reporting procedure from the given doubly-minimal signature and of the results of iteration (until termination) of the left-reporting procedure from the given doubly-minimal signature.

The above procedure can also be performed subject to expense and probe-length limitations, but wherein the enumeration of doubly-minimal signatures may be incomplete when a maximal probe length is reached.

### Approach 5: Design of a Probe-Based Copy Number Assay for Identifying Target-Specificity and Minimal Signatures

Identifying a minimal signature, confirming an ASO is designed from a minimal signature with respect to the intended recipient, and conducting a personalized on-target-off-target check can be reduced to determination of a set of sequences and the overall copy number thereof, that we refer to as regular-expression-targeted sequences (RETS) and RETS-copy-number, respectively. A RETS set of a sequence is the set of sequences that exactly match the CSREE of the sequence or

PSRC of the sequence when the sequence is an ASO. The RETS-copy-number describes the number of times a sequence of the RETS set appears in a reference set of nucleic acids relative to the number of times an appropriately selected control sequence appears.

Here we describe the design of a new type of assay for determining a RETS copy number, which may be implemented using digital PCR as an alternative to using a digital analysis method on the complete set of mRNA sequences. In preparation of the use of digital PCR, a set of probes that exactly covers the set of RETS (or their mRNA-derived cDNAs) are used as well as at least one probe for a control sequence.

We refer to the sequence of a polynucleotide of an mRNA that is transcribed and/or spliced from only one strand of DNA from the set of chromosomes and does not appear at multiple positions with the mRNA as being of singular origin. A positive control sequence is best chosen to be of singular origin to avoid the additional uncertainties associated with control sequences not of singular origin that may lead to improper digital PCR interpretations, misdesigned ASOs, and inadvertent harm.

Choosing a positive control sequence to be of singular origin helps one to best ascertain the copy-number with digital PCR analyses. However, we acknowledge that choosing a positive control sequence to be of singular origin may not always be feasible, for example in a situation of possible familial inbreeding. However, when a positive control sequence is of singular origin, we refer to it as a singleton control sequence.

A singleton control sequence may be selected to be a subsequence of the target sequence that spans at least one exon-exon junction if necessary, and is to be of maximal length wherein the probe designed to report the presence of the singleton control sequence has highly reliable reporting.

A negative control sequence may also be selected to be used for a probe for a negative control sequence such that no sequence of the genome nor of the transcriptome of the intended recipient is expected to or does comprise the control sequence.

The selected digital PCR plate well partitions must be designed to accept equivalent volumes of liquid from the mixture being assayed. Prior to dispensing, the sample mixture must be homogenized (and may contain the probe for the negative control sequence), wherein each well to be used is assigned a single probe (in addition to the probe for the negative control sequence when applicable), wherein the single probe may be assigned to multiple wells, wherein the probe solution/mixture/suspension once dispensed to the otherwise homogenized sample mixture must constitute either a small percentage of the combined (and now heterogeneous) sample mixture or the dispensed quantity must be dispensed with standard deviation of similar small percentage, to mitigate uncertainties that can be introduced by variations of liquid quantities and nucleic acid concentrations, wherein the nucleic acid concentrations are recommended to be enough to fill a majority of partitions, but not so much as to introduce additional uncertainties.

Then, having performed the cycles necessary for digital PCR readout, and given the standard deviations in liquid quantities and nucleic acid concentrations between wells, one may use the analytical methods of digital PCR to count the partitions that are positive for each probed-targeted sequence, estimate the number of copies of probe-targeted sequence in each well, and determine the copy number of each probed RETS well relative to the probed control sequence well(s), with a probe for the negative control sequence that may help with interpretation, and sum the copy numbers associated with the set of RETS to produce the RETS-copy-number.

When the RETS-copy-number is one (1) with respect to the individual recipient's transcriptome and optionally zero (0) with respect to the individual recipient's genome, the degree of uncertainty with the RETS-copy-number calls are acceptable, and the sequence from which the RETS set was defined is a subsequence of the positive control sequence, then one may consider the sequence from which the RETS set was defined to be a signature.

When the RETS-copy-number is one (1) with respect to the individual recipient's transcriptome and optionally zero (0) with respect to the individual recipient's genome, the degree of uncertainty with the RETS-copy-number calls are acceptable, and the sequence from which the RETS set was defined is an ASO whose PSRC is a subsequence of a positive control sequence, then one may consider the sequence from which the RETS set was defined to be a target-specific ASO.

When the RETS-copy-number is one (1) with respect to the individual recipient's mRNA transcriptome, one (1) with respect to the individual recipient's transcriptome, and one (1) with respect to the individual recipient's genome, then one may also consider the sequence (subsequence of positive control sequence or ASO) from which the RETS set was defined to be a signature or target-specific ASO, respectively.

By using the above methods, one may repeat the procedure using probes for RETS sets defined from different length subsequences of the positive control sequence (while retaining the exon-exon junction-crossing property if necessary) to identify a minimal signature.

One skilled in the art shall recognize that it is also possible to perform a RETS-copy-number assay on more than one subsequence of the positive control sequence at a time. For example, one may identify the RETS sets (optionally implicitly, and/or sizes) for a set of subsequences of the positive control sequence, wherein the set of subsequences form a total ordering with respect to the subsequence relation (while retaining the exon-exon junction-crossing property if necessary), and the set of probes needed to cover the RETS sets are neither size nor cost prohibitive, then use the collective set of probes in one or more RETS-copy-number assays to (for example) identify a minimal signature.

A minimal signature is identified by a pair of subsequences (wherein one of the sequences is a proper subsequence of maximum length of the other) of a positive control sequence, wherein the RETS-copy-number of the minimal signature (the longer of the two subsequences) is one (1) in the transcriptome and optionally zero (0) in the genome (or one in the mRNA transcriptome, the full transcriptome, and the genome) and the RETS-copy-number of the sequence that is not a minimal signature is more than one (1) in the transcriptome or the mRNA transcriptome, and optionally more than zero (0) in the genome (or greater than one in the genome when equal to one in the mRNA transcriptome).

We note here that a probe chemistry that signals a match to multiple possible nucleotides may be advantageous to reduce the number and expense of required probes to cover the RETS when the ASO chemistry allows for a nucleotide (analog or alternative) of the ASO to pair with more than one type of nucleotide.

Then, for example, if the subsequences are the suffixes of the positive control sequence, then the minimal signature is left-minimal. Alternatively, if the subsequences are the prefixes of the positive control sequence, then the minimal signature is right-minimal. Then, with another assay round using the prefixes or suffixes of the left-minimal or right-minimal signatures respectfully, a doubly-minimal signature can be identified.

One may also use the RETS-copy-number assay to confirm an ASO corresponds to a minimal signature of the intended recipient, by using as a sample the transcriptome of the intended recipient, using the RETS set for the ASO to confirm that the ASO corresponds to a signature and confirm that the longest proper prefix, longest proper suffix, or both the longest proper prefix and the longest proper suffix of the ASO are not signatures to confirm right-minimality, left-minimality, or doubly-minimality of the signature, respectfully.

A personalized on-target-off-target check for an ASO can be done by confirming that the RETS-copy-number is equal to one (1) for the transcriptome, and optionally zero (0) for the genome, or one (1) for the mRNA transcriptome, the transcriptome, and the genome.

### Approach 6: Probe-Based Sequencing Approach for Identifying Target-Specificity and Minimal Signatures

Another approach to identifying minimal signatures is to use probe-based sequencing, which can provide additional information (the portion of the sequence after the probe) for greater certainty than with assaying for the sequence only the probe was designed for. As described above, one may test whether a target subsequence of the target sequence is a signature by sequencing via a set of probes designed to identify the set of distinct sequences of a reference set that start with a sequence that matches a CSREE of the target sequence.

Because the length of the sequences identified by probe-based sequencing may differ from one another, it can be useful to consider two sequences (inclusive of the probe sequence) produced from probe-based sequencing to be the same if their longest common prefix has length equal to the shorter sequence, while retaining the longer of the two sequences as representative of the two.

Then, after identifying the set of sequences considered distinct (i.e. not the same) via probe-based sequencing in both the genome and the transcriptome, one may rule out the probe-targeted sequence as a signature if the number of the distinct genome sequences produced in total by the probe-based sequencing is greater than one (1), and then increase the length of the probe-targeted sequence and repeat until the number of distinct genome sequence produced is less than or equal to one (1).

If or once there are one or fewer distinct sequences that matches the genome, then it is also useful to reduce the set of sequences identified from the transcriptome further based on splicing information. To reduce the set of sequences further, we remove the distinct sequence from the transcriptome-based set that is considered the same (T's substituted for U's) as the distinct sequence from the genome-based set, to form a residual transcriptome-based set.

Then, if genome-based set is of size one (1), then the sequences of the residual transcriptome-based set are to be aligned with the sequence of the genome-based set to determine whether the transcriptome-based sequences are each plausible splices of genomic sequence. It is noted here that depending on the length of the genome-based sequence, the genome-based sequence may need to be extended by further sequencing. It is also noted here that depending on the reliability of the genome-based sequencing method, it may be necessary to repeat the probe-based sequencing to distinguish possible errors from legitimate variation.

If the residual transcriptome sequences splice plausibly do not splice to more than the number of origins in the target mRNA sequence in the genome-based sequence, and the plausibility of residual variation being due to genome variation is acceptably low, then the probe-targeted sequence can be considered a signature.

Then performing a method of approach 3 or 4 above that tests further subsequences of the probe-targeted sequence can help identify minimal signatures.

However, if there are no probe-based sequences that match the genome, but do match the transcriptome, then it may be that the probe-targeted sequence is a signature of an exon-exon junction, which may be further analyzed as above, or another probe-targeted sequence can be chosen.

Similarly to the above, the personalized on-target-off-target check can be performed by checking whether the PSRC of the ASO is a signature. If the PSRC of the ASO is a signature and targets the intended target mRNA sequence as determined via computational homology of the target-sequence to the reference for the species, then the ASO can be considered on-target and not off-target. Furthermore, if the PSRC of the ASO identifies a signature and the neither the longest proper prefix nor longest proper suffix of the target subsequence (CSRC that produced the ASO) is a signature, then the ASO corresponds to a minimal signature of the intended recipient.

### Digitally Identifying Pareto-Specific ASOs and Maximally-Specific ASOs

An example method embodiment to compute target-specific ASOs from identified minimal signatures is provided here.

Let us define the notion of context-sensitive reverse complement (CSRC) of a nucleotide to be a function such that (i,j) in CSRC when (i,t) in CSREE and (j,s) in PSRC and s=t. An example definition of a CSRC using the PSRC and CSREE defined above for convenience is as follows:

CSRC(A)=T when CSREE(A)=A
CSRC(A)=U when CSREE(A)=[AG]
CSRC(C)=G when CSREE(C)=[CU]
CSRC(G)=U when CSREE(G)=[AG]
CSRC(G)-C when CSREE(G)=G
CSRC(T)=A when CSREE(T)=T
CSRC(U)=G when CSREE(U)=[CU]
CSRC(U)=A when CSREE(U)=[UT]

Then the CSRC of a signature represents a target-specific ASO with respect to the CSREE and reference set. The context-sensitive reverse complement (CSRC) of a polynucleotide sequence S=s₁,s₂, . . . ,sₖ₋₁,sₖ is defined with respect to the CSREE, as CSRC(S)=CSRC(sₖ), CSRC(sₖ₋₁), . . . , CSRC(s₂),CSRC(s₁). In another important note, please note that CSRC(sᵢ) need not equal CSRC(sⱼ) when sᵢ=sⱼ but CSREE(sᵢ) does not equal CSREE(sⱼ).

### The Use of an Appropriate Pairing-Sensitive CSREE May Help Mitigate Against Off-Target Binding and Potentially Linked Disastrous Health Outcomes or Morbidities.

The above procedures of this disclosure are to be used with the pairing-sensitive CSREE and corresponding CSRC, to report sequences for MS-ASOs. When doing so, the CSRC of each pairing-sensitive right-minimal signature is reported as a target-specific ASO for the target sequence that does not have a proper suffix that is also a target-specific ASO sequence. The CSRC of any pairing-sensitive left-minimal signature is reported as a target-specific ASO for the target sequence that does not have a proper prefix that is also a target-specific ASO for the target sequence. Consequently, the CSRC of any pairing-sensitive doubly-minimal signature is reported as a maximally-specific ASO (MS-ASO) for the target sequence.

We refer to an ASO that is the CSRC of one of the three types of minimal signatures as pareto-specific. Furthermore, it is worth noting here that the PSRC of a pareto-specific ASO accepts at least one minimal signature of the target sequence with respect to a CSREE.

### Personalized Health Applications and Pipelines

### Marker Design, Sequencing, and Screening

In addition to the above methods, an initial detection or screening of a signature can be done by many approaches, methods, and/or tools, including but not limited to finding the sequence or a reverse complement in DNA of one or more genomes, finding the sequence in RNA of one or more transcriptomes, and/or finding the encoded protein; leveraging read-out or detection techniques such as sequencing technologies and/or marker technologies; applying one or more biochemistries that quantitate or signal presence through fluorescence or other means; and/or using signal-enhancing methods including but not limited to PCR, digital PCR, endpoint PCR, and/or real-time PCR. One skilled in the art shall recognize that detected sequences are sometimes referred to as reads (or subsequences thereof).

The detection of a variant (often referred to as an allele) signature may be used to identify associated pathogenic predisposition(s). However, the use of DNA detection methods can help assure quality, for example by designing a marker such that it also detects and/or quantifies the presence of a sequence present in reference set DNA. For an initial screening, we may refer to the chosen reference set DNA subsequence as a control sequence.

The control sequence is typically chosen by the marker designer to be a control gene (usually on a different chromosome than the targeted allele) or to be an alternate allele (usually sharing a locus with the target allele signature and differing from the target allele signature by typically one, but allowably more than one, nucleotide). The control gene and/or alternate allele sequence is typically chosen such that it appears co-located once per reference chromosome set, in order to simplify interpretation (referred to as scoring or “marker calling”). One skilled in the art shall recognize that reducing the genetic distance between the loci of the target and alternate alleles can generally help reduce type I error and type II error.

An initial development of candidate diagnostic markers can be done by substituting each pathogenic variant into a non-pathogenic mRNA sequence and then identifying subsequence(s) of the pathogenic mRNA sequence version that do not appear in the non-pathogenic reference set of the species or a specimen thereof composed of either a reference transcriptome, a reference genome, or both. We refer to a subsequence of the pathogenic mRNA sequence version that does not otherwise appear in the selected reference set as a pathogenic signature.

One skilled in the art shall recognize there are multiple and substantially equivalent approaches to find a pathogenic signature, for example including but not limited to, searching for individual subsequences of the pathogenic mRNA sequence in the reference set sequences, searching for their reverse complements, using regular expression pattern matching in a sequence, a graph, or an index thereof.

One skilled in the art shall recognize variants of, deterministic finite automata (DFA) of, and/or decorated versions of suffix arrays, suffix trees, their extensions, generalized versions, and augmentations such as data structures for longest common prefix (LCP) queries, inverted suffix arrays, and/or even substring search can suffice, can assist with the search.

In one example approach, one may produce a suffix tree and suffix links from a suffix array and its LCP array from the reference set and use the suffix tree to check successively larger prefixes of a query string from the pathogenic mRNA sequence until the substring of the query does not exist in the suffix tree, at which point the string can be considered a right minimal signature and then use suffix links to consider the suffixes in order of decreasing length to test for double-minimality if desired, such as by using approaches described above for finding minimal signatures.

Then the identified set of candidate pathogenic signatures may be further analyzed and refined and markers can be designed therefrom to comprise a pathogenic signature probe together with a probe that detects a non-pathogenic variant of the reference set.

Now, we refer to a read or detected sequence that contains a pathogenic signature as a pathogenic read or pathogenic sequence. We say a pathogenic genetic condition is indicated by a pathogenic sequence when a contained pathogenic signature traverses a path over an allele (or variant) that is considered pathogenic.

Pathogenic reads and signatures can help detect the presence of a pathogenic predisposition and can help determine the identity of the pathogenic predispositions by location of each read within a reference set. Consequently, one or more DNA assays and/or one or more RNA assays can be used together with the aforementioned procedures to identify the presence of a pathogenic signature if one exists.

Furthermore, one skilled in the art shall understand that a reference set with unrepresented relevant variation may cause spurious detections or interfere with detection of a targeted variant with a marker. Therefore, detection of a pathogenic signature is necessary but not sufficient to assure safety and efficacy, we must also assure locality of the detection (and optionally so-called copy number), and sequencing can be useful in doing so. One may use BLAST or similar tool with reference of the species to confirm homology of a candidate pathogenic read.

### Phenotype-Driven Personalized Treatment and Supplemental Fortification

We say a supplemented nucleic acid is a pathogenicity-specific supplemental polynucleotide with respect to a detected pathogenic sequence when the nucleic acid induces a protein analogous to the non-pathogenic reference protein induced by a non-pathogenic reference allele associated with the locus uniquely pinpointed by the detected pathogenic sequence, but not having any pathogenic signature. In addition, we say an ASO is a suppressive polynucleotide for a detected pathogenic sequence when a pathogenic signature contained by the pathogenic sequence is accepted by the PSRC of the ASO. However, we only say the ASO is a pathogenicity-specific suppressive polynucleotide when the ASO is target-specific for the pathogenic sequence with respect to the intended recipient's transcriptome.

We note here that in some situations (for example if a G may pair with a U) there may not exist a pathogenicity-specific suppressive for a pathogenic sequence variant. One such situation may be in the case a G can pair with a U and there is a DNA mutation that is a pathogenic C in place of a non-pathogenic T on the transcriptional strand (and a pathogenic G in place of a non-pathogenic A on the non-transcriptional strand). Under the same assumptions, and the site of variation is known, it is also reasonable to avoid CSRC(U)=G when the effect of a DNA mutation is a pathogenic T in place of a non-pathogenic C on the transcriptional strand (a pathogenic A in place of a non-pathogenic G on the non-transcriptional strand), and to avoid CSRC(G)=U when the effect of a DNA mutation is a pathogenic A or G in place of a non-pathogenic G or A, respectively, on the transcriptional strand (a pathogenic T or C in place of a non-pathogenic C or T, respectively, on the non-transcriptional strand).

When a present pathogenic genotype is expressed, the above procedures can be used to calculate, design, or lookup one or more sequence designs of pathogenicity-specific polynucleotides associated with the pinpointed locus and/or traversed pathogenic variant to compose the polynucleotides(s) of one or more personalized treatments. However, to further assure efficacy, phenotypic information about the nature of the specific disease can be used to further refine the personalized treatment composition.

To determine the appropriateness of treatment for a pathogenic predisposition, it can be helpful to determine whether the pathogenic genetic condition is caused by or amplified by non-presence of a functional transcript (i.e. a disease caused by the absence of the associated protein) and/or caused by or amplified by an actively disruptive transcript (i.e. the associated protein blocks a helpful pathway or creates a problematic pathway) and/or caused by a quantitative insufficiency (haploinsufficiency) and/or co-expression imbalance.

If the genetic disease is caused by non-presence of a functional transcript, then an appropriate modality of treatment is to include a pathogenicity-specific supplemental polynucleotide. If the pathogenic transcript is actively disruptive, then an appropriate modality of treatment is to include a pathogenicity-specific suppressive polynucleotide. In some circumstances of co-expression imbalance without quantitative insufficiency a co-expression rebalancing treatment may be appropriate, wherein one of two variants (i.e. on one of the two haploids in diploidy) of each gene requiring balance in co-expression is suppressed. In some circumstances of haploinsufficiency where overexpression is not pathogenic itself, a pathogenicity-specific supplemental polynucleotide may be the appropriate modality.

We note here that in some situations a therapeutic comprising a pathogenic-specific suppressive polynucleotide may benefit from inclusion of a corresponding pathogenicity-specific supplemental polynucleotide. We refer to a pathogenic-specific supplemental polynucleotide as fortified against an ASO, when the pairing-sensitive reverse complement of the ASO does not accept a subsequence of the pathogenic-specific supplemental nor any RNA transcribed and/or spliced therefrom. To design a pathogenic-specific supplemental polynucleotide to be fortified against an ASO, one may make one or more codon substitutions, where a substitution consists of substituting one codon for another codon, in a sequence where the two codons encode the same amino acid.

When a pathogenic phenotype indicated by a pathogenic read is autosomal recessive or X-linked recessive, the appropriate modality of the personalized nucleic acid treatment is the pathogenicity-specific supplemental polynucleotide(s). When a pathogenic phenotype indicated by a pathogenic read is autosomal dominant or X-linked dominant, but not due to haploinsufficiency or co-expression imbalance, the appropriate modality of the personalized nucleic acid treatment comprises the pathogenicity-specific suppressive polynucleotide(s). When a pathogenic phenotype indicated by a pathogenic read is Y-linked, cytoplasmic inheritance, or incomplete-dominance, the appropriate modality of the personalized nucleic acid treatment recommendation is the pathogenicity-specific supplemental polynucleotide, pathogenicity-specific suppressive polynucleotide, or both types of pathogenicity-specific polynucleotides, when the pathogenic condition is caused by a missing functional nucleic acid, an actively disruptive transcript, or both, respectively.

We refer to an appropriate treatment as supplementally-mending when it only includes pathogenicity-specific supplemental polynucleotides. We refer to an appropriate treatment as suppressively-mending when it only requires pathogenicity-specific suppressive polynucleotides and the pathogenic phenotype is not due to haploinsufficiency. We refer to an appropriate treatment as multimodal-mending when both pathogenicity-specific supplemental polynucleotide(s) and pathogenicity-specific suppressive polynucleotide(s) are required, such as in some situations including but not limited to incomplete dominance, polygenicity, and/or epistasis.

Other factors such as but not limited to tissue/cell targeting, dosing, timing, duration, and non-interruption of treatment may also be important considerations for safety and efficacy, as genetic diseases may be specific to certain tissues and/or cell types, sensitive to relative quantities of transcripts, be congenital, may develop over time after birth or later in life, or provide other phenotypic clues of importance to guide formulation (e.g. decoration by transporter and/or HLA proteins) and timing (e.g. in utero, developmental, or chronic) of treatment.

Decoration by transporter-specific proteins may aid in the specificity of delivery to the relevant tissues and/or cell types. Decoration by autologous human leukocyte antigen (HLA) proteins may aid in averting an immune response by the recipient. For congenital genetic conditions, for example, such as autosomal dominant polycystic kidney disease, preventative gestational/in utero treatment may be required to benefit the recipient. For developmental genetic diseases, for example, such as Marfan syndrome or familial amyloid polyneuropathy, preventative treatment during development may be required to benefit the recipient. And for chronic genetic disease, for example, cystic fibrosis and Tay-Sachs disease, sustained treatment may be required to benefit the recipient.

TABLE 4
Gene Training for Application to Personalized Health

| a) | Index pathogenic variants and associated phenotypes in database. | |
|---|---|---|
| b) | Obtain genome, transcriptome, and/or exome of intended treatment recipient. | |
| c) | Traverse database or screen sequenced polynucleotides for pathogenic signature(s). | |
| d) | Identify HLA variants from sequenced polynucleotides (or proteome). | |
| e) | Computer aided design and/or lookup of recommended mending treatment(s). | |
| | a. | Recommendation to include HLAs, ASO(s), supplemental mRNA(s) and any |
| | | fortifications. |
| | b. | Personalized on-target check. |
| f) | Optionally, an operator may add, select, and modify design elements such as: | |
| | a. | Use of polynucleotide analogs, adjustment of percentage t, extension of ASO(s), |
| | | fortifications, refinement of HLAs, delivery mechanism (e.g. vesicle, LNP, |
| | | polynucleotide structure). |
| | b. | Personalized on-target check. |

| | |
|---|---|
| g) | Each treatable genetic pathogenicity of sufficient concern to be treated via at least one |
| | (m)RNA variant and/or (MS−)ASO to supplement non-pathogenic variant(s) and/or suppress |
| | pathogenic variant(s), respectively. |

### EXAMPLES

For convenience and to avoid obscuring the disclosure, we use negative MFE (−MFE) of the coding region of sequence S as the estimator EL(S) in the examples that follow, using CDSFold and RNAFold. However, as mentioned previously there are many additional potential definitions of estimators and optimization algorithms, such as but not limited to difference percentages, −MFE of the mRNA sequence containing the CDS, the −MFE of the primary RNA transcript sequence from which the mRNA is spliced, and/or a composition or product of functions of multiple estimators. In addition, the examples and embodiments of methods of this disclosure are not to be construed as limited to a particular example, estimator, difference ordering, gene, protein, transcript, splice, genome, transcriptome, annotation, trait, phenotype, crop, livestock, genetic disease, species, person, or population.

### A. Example 1: Gene Training for Production Crop Improvement

In this example, we focus on design of a decrease in expression of an existing gene in a maize inbred toward decreasing plant stature as a step towards increased stalk strength. Increased stalk strength in maize can increase crop resistance to wind events both during seed production, but also during grain production. In this example, the Zm00001eb366090_T001 splice sequence of GA20ox3 (i.e. gibberellin 20-oxidase 3 aka gibberellin 20-oxidase 5) from the B73 reference is trained to a lower relative expression design level.

Example Protein Sequence (GA20ox3, e.g. Zm00001eb366090_P₀₀₁):

SEQ ID NO: 1 provides the amino acid sequence of the Zm00001eb366090_P001 variant of a
GA20ox3 protein of Zea mays
MRPRLPPNVPSLPSSLSLLANSLSSPVTNTPTRPDSFPAYLQLAHLMVSQERQEPAVPSSSSSSAKRAATSMDASPAPPLLLR
APTPSPSIDLPAGKDKADAAASKAGAAVFDLRREPKIPAPFLWPQEEARPSSAAELEVPMVDVGVLRNGDRAGLRRAAAQVAA
ACATHGFFQVCGHGVDAALGRAALDGASDFFRLPLAEKQRARRVPGTVSGYTSAHADRFAAKLPWKETLSFGYHDGAASPVVV
DYFVGTLGQDFEPMGWVYQRYCEEMKELSLTIMELLELSLGVELRGYYREFFEDSRSIMRCNYYPPCPEPERTLGTGPHCDPT
ALTILLQDDVGGLEVLVDGEWRPVRPVPGAMVINIGDTFMALSNGRYKSCLHRAVVNQRRARRSLAFFLCPREDRVVRPPASA
APRRYPDFTWADLMRFTQRHYRADTRTLDAFTRWLSHGPAQAAAPPCT

Upper Calibrating GA20ox3 CDS RNA Sequence (For Calibration via CDSFold, −MFE=1145; [0-1] design level=100%):

SEQ ID NO: 2 provides a design for a nucleic acid CDS sequence trained to a 100% expression
design level encoding of the Zm00001eb366090_P001 variant of a GA20ox3 protein of Zea mays
AUGCGCCCCCGGCUCCCGCCGAACGUGCCGUCACUCCCCAGCUCCUUGAGUCUCUUGGCGAACUCCCUGAGUAGCCCCGUGAC
CAACACCCCGACUCGGCCCGACAGCUUCCCUGCAUACUUGCAGCUGGCUCACCUAAUGGUGAGCCAGGAGCGGCAGGAGCCGG
CGGUGCCGUCCAGCUCGUCGUCGUCCGCAAAGCGGGCGGCGACGAGCAUGGACGCAUCGCCGGCUCCGCCGCUCCUGCUGCGU
GCACCCACCCCAUCGCCCUCGAUAGACCUGCCCGCGGGUAAAGACAAAGCGGACGCCGCCGCCUCCAAAGCGGGGGCGGCGGU
GUUCGACCUGCGGCGUGAGCCGAAGAUCCCGGCACCGUUCCUCUGGCCCCAGGAGGAGGCGCGACCCUCGUCGGCCGCAGAGC
UGGAAGUGCCCAUGGUGGACGUAGGCGUGCUGCGAAAUGGGGACCGCGCGGGGCUACGCCGCGCGGCUGCCCAGGUCGCAGCC
GCCUGCGCCACCCAUGGGUUCUUCCAGGUCUGCGGCCACGGGGUCGACGCCGCCCUGGGGCGAGCGGCACUCGACGGGGCCUC
UGACUUCUUCCGCCUACCUUUGGCGGAGAAGCAGAGGGCCCGUCGAGUGCCGGGAACGGUGUCGGGAUAUACUUCGGCUCACG
CCGACAGGUUCGCGGCAAAACUGCCGUGGAAGGAGACACUCUCCUUCGGCUACCACGACGGGGCGGCUAGCCCCGUCGUGGUA
GACUACUUUGUCGGUACCCUCGGGCAGGACUUCGAGCCGAUGGGGUGGGUGUACCAGCGGUAUUGUGAGGAGAUGAAAGAAUU
AUCCCUCACAAUAAUGGAGCUGUUGGAGCUGAGUCUGGGUGUUGAGUUACGGGGCUACUACAGGGAGUUCUUCGAGGACUCAA
GGAGCAUAAUGCGGUGUAAUUAUUAUCCCCCGUGCCCAGAGCCGGAACGGACUCUGGGCACGGGGCCGCAUUGUGAUCCAACA
GCACUGACAAUCCUCCUACAGGACGAUGUAGGAGGAUUGGAGGUGCUGGUGGAUGGGGAGUGGCGGCCCGUUCGGCCGGUGCC
GGGGGCGAUGGUCAUAAACAUUGGAGACACGUUUAUGGCCCUGAGUAAUGGGCGGUAUAAAAGCUGCCUGCACCGGGCCGUGG
UCAACCAGCGCCGAGCGCGUCGGAGCCUGGCGUUCUUCCUGUGUCCGCGGGAGGACCGCGUGGUCCGCCCUCCGGCUAGCGCC
GCACCGCGGCGCUAUCCGGAUUUUACUUGGGCGGACCUAAUGCGGUUCACCCAGCGGCACUACAGGGCGGACACCAGGACUCU
CGACGCGUUCACGCGCUGGUUGAGCCACGGCCCGGCGCAGGCAGCUGCGCCGCCCUGUACUUAG

Lower Calibrating GA20ox3 CDS RNA Sequence (For Calibration via CDSFold, −MFE=203; [0-1] design level=0%):

SEQ ID NO: 3 provides a design for a nucleic acid CDS sequence trained to a 0% expression
design level encoding of the Zm00001eb366090_P001 variant of a GA20ox3 protein of Zea mays
AUGAGACCAAGAUUACCACCAAAUGUACCAUCAUUACCAUCAUCAUUAUCAUUAUUAGCAAAUUCAUUAUCAUCACCAGUAAC
AAAUACACCAACAAGACCAGAUUCAUUUCCAGCAUAUUUACAAUUAGCACAUUUAAUGGUAUCACAAGAAAGACAAGAACCAG
CAGUACCAUCAUCAUCAUCAUCAUCAGCAAAAAGAGCAGCAACAUCAAUGGACGCAUCACCAGCACCACCAUUAUUAUUAAGA
GCACCAACACCAUCACCAUCAAUAGAUUUACCAGCAGGAAAAGAUAAAGCAGAUGCAGCAGCAUCAAAAGCAGGAGCAGCAGU
AUUUGAUUUAAGAAGAGAACCAAAAAUACCAGCACCAUUUUUAUGGCCACAAGAAGAAGCAAGACCAUCAUCAGCAGCAGAAU
UAGAAGUACCAAUGGUAGAUGUAGGAGUAUUAAGAAAUGGAGAUAGAGCAGGAUUAAGAAGAGCAGCAGCACAAGUAGCAGCA
GCAUGUGCAACACAUGGAUUUUUUCAAGUAUGCGGACAUGGAGUAGAUGCAGCAUUAGGAAGAGCAGCAUUAGACGGAGCAUC
AGAUUUUUUUAGAUUACCAUUAGCAGAAAAACAAAGAGCAAGAAGAGUACCAGGAACAGUAUCAGGAUAUACAUCAGCACAUG
CAGAUAGAUUUGCAGCAAAAUUACCAUGGAAAGAAACAUUAUCAUUUGGAUAUCAUGACGGAGCAGCAUCACCAGUAGUAGUA
GAUUAUUUUGUAGGAACAUUAGGACAAGAUUUUGAACCAAUGGGAUGGGUAUAUCAAAGAUAUUGUGAAGAAAUGAAAGAAUU
AUCAUUAACAAUAAUGGAAUUAUUAGAAUUAUCAUUAGGAGUAGAAUUAAGAGGAUAUUAUAGAGAAUUUUUUGAAGAUUCAA
GAUCAAUAAUGAGAUGUAAUUAUUAUCCACCAUGUCCAGAACCAGAAAGAACAUUAGGAACAGGACCACAUUGCGAUCCAACA
GCAUUAACAAUAUUAUUACAAGAUGAUGUAGGAGGACUAGAAGUAUUAGUAGACGGAGAAUGGAGACCAGUAAGACCAGUACC
AGGAGCAAUGGUAAUAAAUAUAGGAGAUACAUUUAUGGCAUUAUCAAAUGGAAGAUAUAAAUCAUGUUUACAUAGAGCAGUAG
UAAAUCAAAGAAGAGCAAGAAGAUCAUUAGCAUUUUUUUUAUGUCCAAGAGAAGAUAGAGUAGUAAGACCACCAGCAUCAGCA
GCACCAAGAAGAUAUCCAGAUUUUACAUGGGCAGAUUUAAUGAGAUUUACACAAAGACAUUAUAGAGCAGAUACAAGAACAUU
AGAUGCAUUUACAAGAUGGUUAUCACAUGGACCAGCACAAGCAGCAGCACCACCAUGUACAUAA

Example Reference GA20ox3 CDS RNA Sequence (Reference Level, −MFE-706; [0-1] design level=53.4%=(706-203)/(1145-203)

SEQ ID NO: 4 provides the nucleic acid sequence of the Zm00001eb366090_T001 CDS
encoding of the Zm00001eb366090_P001 variant of a GA20ox3 protein of Zea mays
AUGAGGCCGCGCCUCCCUCCAAAUGUUCCCUCCCUGCCUUCGUCUUUGUCGUUGCUCGCAAACUCCCUGUCCUCCCCUGUUAC
AAAUACCCCCACCCGCCCGGACAGCUUCCCUGCAUACUUGCAGCUCGCACAUCUCAUGGUGUCGCAGGAACGACAAGAGCCAG
CUGUGCCUAGCAGCAGCAGCAGCAGCGCCAAGCGCGCAGCCACGUCCAUGGACGCCAGCCCGGCCCCGCCGCUCCUCCUCCGC
GCCCCCACUCCCAGCCCCAGCAUUGACCUCCCCGCUGGCAAGGACAAGGCCGACGCGGCGGCCAGCAAGGCCGGCGCGGCCGU
GUUCGACCUGCGCCGGGAGCCCAAGAUCCCCGCGCCAUUCCUGUGGCCGCAGGAAGAGGCGCGGCCGUCCUCGGCCGCGGAGC
UGGAGGUGCCGAUGGUGGACGUGGGCGUGCUGCGCAAUGGCGACCGCGCGGGGCUGCGGCGCGCCGCGGCGCAGGUGGCCGCG
GCGUGCGCGACGCACGGGUUCUUCCAGGUGUGCGGGCACGGCGUGGACGCGGCGCUGGGGCGCGCCGCGCUGGACGGCGCCAG
CGACUUCUUCCGGCUGCCGCUCGCCGAGAAGCAGCGCGCCCGGCGCGUCCCCGGCACCGUGUCCGGGUACACGAGCGCGCACG
CCGACCGGUUCGCGGCCAAGCUCCCCUGGAAGGAGACCCUGUCGUUCGGCUACCACGACGGCGCCGCGUCGCCUGUCGUCGUG
GACUACUUCGUCGGCACCCUCGGCCAGGAUUUCGAGCCAAUGGGGUGGGUGUACCAGAGGUACUGCGAGGAGAUGAAGGAGCU
GUCGCUGACGAUCAUGGAGCUGCUGGAGCUGAGCCUGGGCGUGGAGCUGCGCGGCUACUACCGGGAGUUCUUCGAGGACAGCC
GGUCCAUCAUGCGGUGCAACUACUACCCGCCGUGCCCGGAGCCGGAGCGCACGCUGGGCACGGGCCCGCACUGCGACCCCACG
GCGCUCACCAUCCUCCUGCAGGACGACGUGGGCGGGCUGGAGGUGCUGGUGGACGGUGAGUGGCGCCCCGUCCGGCCCGUCCC
GGGCGCCAUGGUCAUCAACAUCGGCGACACCUUCAUGGCGCUGUCGAACGGGAGGUACAAGAGCUGCCUGCACCGCGCGGUGG
UGAACCAGCGGCGGGCGCGGCGGUCGCUGGCCUUCUUCCUGUGCCCGCGCGAGGACCGGGUGGUGCGCCCGCCGGCCAGUGCU
GCGCCGCGGCGCUACCCGGACUUCACCUGGGCCGACCUCAUGCGCUUCACGCAGCGCCACUACOGCGCCGACACCCGCACGCU
GGACGCCUUCACCCGCUGGCUCUCCCACGGCCCGGCCCAGGCGGCGGCGCCUCCCUGCACCUAG

If the desired training [0-1] design level=45%, we might choose to use the Reference CDS RNA Sequence as the Primary Bounding Sequence ([0-1] design level=53.4%) and the Lower Calibrating CDS RNA Sequence ([0-1] design level=0%) as the Ultimate Bounding Sequence ([0-1] design level=0%) for training expression. When using base or prime editing, choosing the Reference CDS RNA Sequence as the Primary Bounding Sequence can reduce the number of polynucleotide positions necessary to edit.

With the PBS at 53.4% and the UBS at 0%, our desired expression level is 45%, which is 84.3% between the selected UBS and PBS levels.

When defining the difference at the codon resolution, the triplet indices shown below (one-based indexing) are the set of differences from the PBS to the UBS. In this example, we use the descending contiguous ordering of the differences.

464 463 462 461 460 459 458 457 456 455 454 453 452 451 450 448 447 446 445 444 443
442 441 440 439 438 437 436 435 434 433 432 431 430 428 427 426 424 423 422 421 420
419 418 417 416 415 414 413 412 411 410 409 408 407 406 405 404 403 402 401 400 399
398 397 396 395 394 393 392 391 390 389 388 387 386 385 384 383 382 381 380 379 378
377 376 375 374 373 371 370 369 368 367 366 365 364 362 361 360 359 358 357 356 355
354 352 351 349 348 347 346 345 344 343 342 341 340 339 338 337 336 335 334 333 332
331 330 328 327 326 325 324 323 322 321 320 319 318 317 316 315 314 313 312 311 310
309 307 306 305 304 303 302 301 300 299 298 297 296 295 294 293 292 291 290 289 288
287 286 285 284 283 281 280 279 278 277 276 275 273 272 271 270 269 268 267 266 264
261 260 258 257 256 255 254 253 252 251 250 249 248 247 246 245 244 243 242 240 239
238 237 236 235 234 233 232 230 229 228 227 226 225 224 223 222 221 220 219 218 217
216 215 214 213 212 211 210 209 208 207 206 205 204 203 202 201 200 199 198 197 196
195 194 193 192 190 189 188 187 186 185 184 183 182 181 180 179 178 176 175 174 173
172 171 170 169 168 167 166 165 164 163 162 161 160 159 158 157 156 155 154 153 152
150 149 148 147 146 145 144 142 141 140 139 138 137 136 135 134 133 132 131 130 128
127 125 124 122 121 120 119 118 117 116 115 114 113 112 111 110 109 108 107 106 105
104 103 102 101 100 99 98 97 96 95 94 93 92 91 90 89 88 87 86 85 84 83 82 81 80 79
78 77 76 75 74 71 70 69 67 66 65 64 63 62 61 60 59 58 57 56 54 52 50 49 48 46 43 42
41 40 38 37 36 35 34 33 32 31 30 27 26 25 24 23 22 21 19 18 17 16 15 14 13 12 11 10
9 6 5 4 3 2

Given the difference above, an example of a (descending contiguous) partial difference PD₃₈₆₋ can be described by the triplet indices numbered lesser than or equal to 386. This partial difference gives −MFE=628, which is 84.4% between the selected UBS and PBS levels. Another example of a (contiguous) partial difference PD₃₈₄₋ can be described by the triplet indices numbered lesser than or equal to 384. This second partial difference gives −MFE=626, which is 84.1% between the selected UBS and PBS levels. Together, the window [84.1%, 84.4%] is a narrow window for the example protein with respect to the UBS and PBS, giving us the expression design level narrow window of [44.9%, 45.1%] with respect to the calibrating sequences encoding the protein sequence.

The implied coding sequence ICS(PD₃₈₆₋), with trained expression level 45.1%, is shown below with the difference from the Reference CDS RNA Sequence underlined:

SEQ ID NO: 5 provides a design for a nucleic acid CDS sequence trained to a 45.1% expression
design level encoding of the Zm00001eb366090_P001 variant of a GA20ox3 protein of Zea mays
AUGAGGCCGCGCCUCCCUCCAAAUGUUCCCUCCCUGCCUUCGUCUUUGUCGUUGCUCGCAAACUCCCUGUCCUCCCCUGUUAC
AAAUACCCCCACCCGCCCGGACAGCUUCCCUGCAUACUUGCAGCUCGCACAUCUCAUGGUGUCGCAGGAACGACAAGAGCCAG
CUGUGCCUAGCAGCAGCAGCAGCAGCGCCAAGCGCGCAGCCACGUCCAUGGACGCCAGCCCGGCCCCGCCGCUCCUCCUCCGC
GCCCCCACUCCCAGCCCCAGCAUUGACCUCCCCGCUGGCAAGGACAAGGCCGACGCGGCGGCCAGCAAGGCCGGCGCGGCCGU
GUUCGACCUGCGCCGGGAGCCCAAGAUCCCCGCGCCAUUCCUGUGGCCGCAGGAAGAGGCGCGGCCGUCCUCGGCCGCGGAGC
UGGAGGUGCCGAUGGUGGACGUGGGCGUGCUGCGCAAUGGCGACCGCGCGGGGCUGCGGCGCGCCGCGGCGCAGGUGGCCGCG
GCGUGCGCGACGCACGGGUUCUUCCAGGUGUGCGGGCACGGCGUGGACGCGGCGCUGGGGCGCGCCGCGCUGGACGGCGCCAG
CGACUUCUUCCGGCUGCCGCUCGCCGAGAAGCAGCGCGCCCGGCGCGUCCCCGGCACCGUGUCCGGGUACACGAGCGCGCACG
CCGACCGGUUCGCGGCCAAGCUCCCCUGGAAGGAGACCCUGUCGUUCGGCUACCACGACGGCGCCGCGUCGCCUGUCGUCGUG
GACUACUUCGUCGGCACCCUCGGCCAGGAUUUCGAGCCAAUGGGGUGGGUGUACCAGAGGUACUGCGAGGAGAUGAAGGAGCU
GUCGCUGACGAUCAUGGAGCUGCUGGAGCUGAGCCUGGGCGUGGAGCUGCGCGGCUACUACCGGGAGUUCUUCGAGGACAGCC
GGUCCAUCAUGCGGUGCAACUACUACCCGCCGUGCCCGGAGCCGGAGCGCACGCUGGGCACGGGCCCGCACUGCGACCCCACG
GCGCUCACCAUCCUCCUGCAGGACGACGUGGGCGGGCUGGAGGUGCUGGUGGACGGUGAGUGGCGCCCCGUCCGGCCCGUCCC
GGGCGCCAUGGUCAUCAACAUCGGCGACACCUUCAUGGCGCUGUCGAACGGGAGGUACAAGAGCUGCCUGCACCGCGCAGUAG
UAAAUCAAAGAAGAGCAAGAAGAUCAUUAGCAUUUUUUUUAUGUCCAAGAGAAGAUAGAGUAGUAAGACCACCAGCAUCAGCA
GCACCAAGAAGAUAUCCAGAUUUUACAUGGGCAGAUUUAAUGAGAUUUACACAAAGACAUUAUAGAGCAGAUACAAGAACAUU
AGAUGCAUUUACAAGAUGGUUAUCACAUGGACCAGCACAAGCAGCAGCACCACCAUGUACAUAA

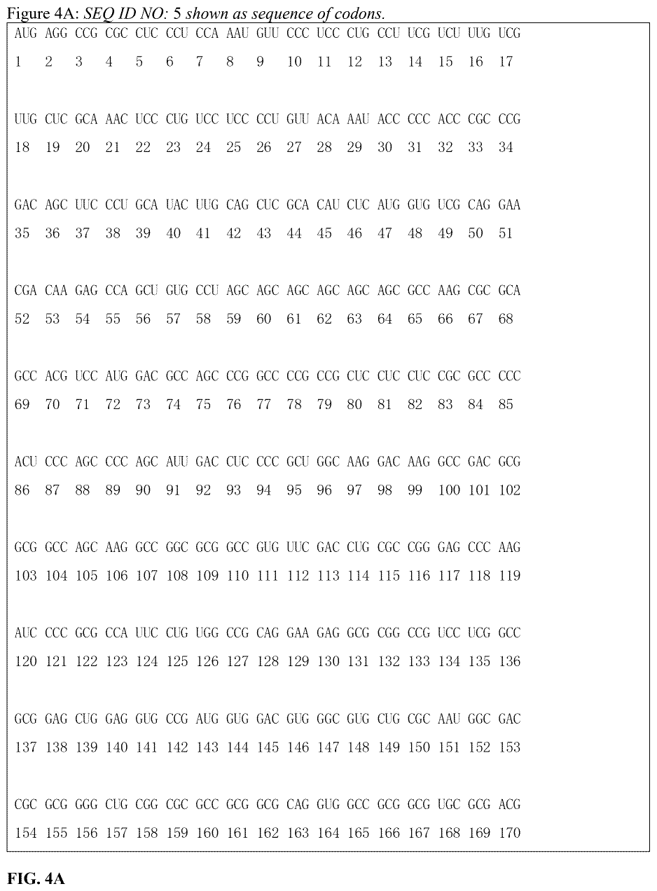

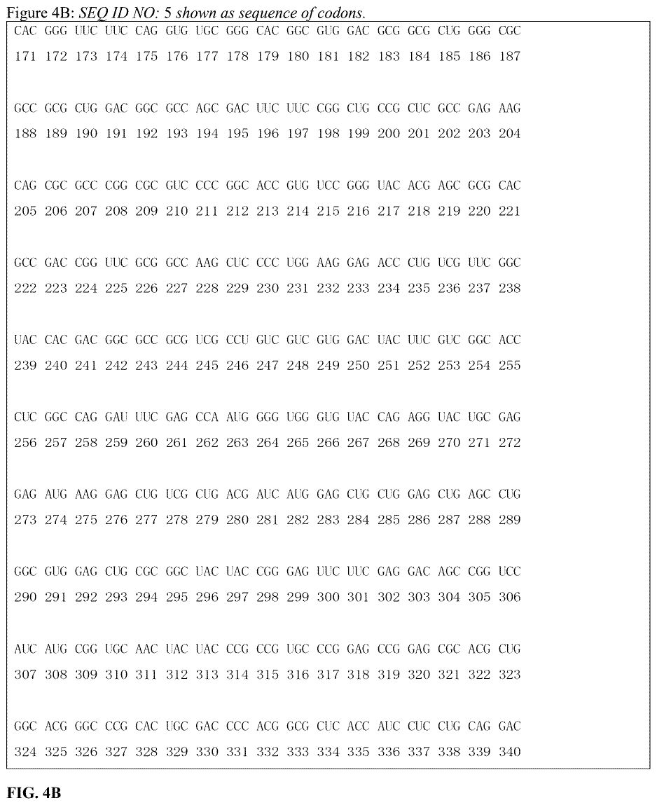

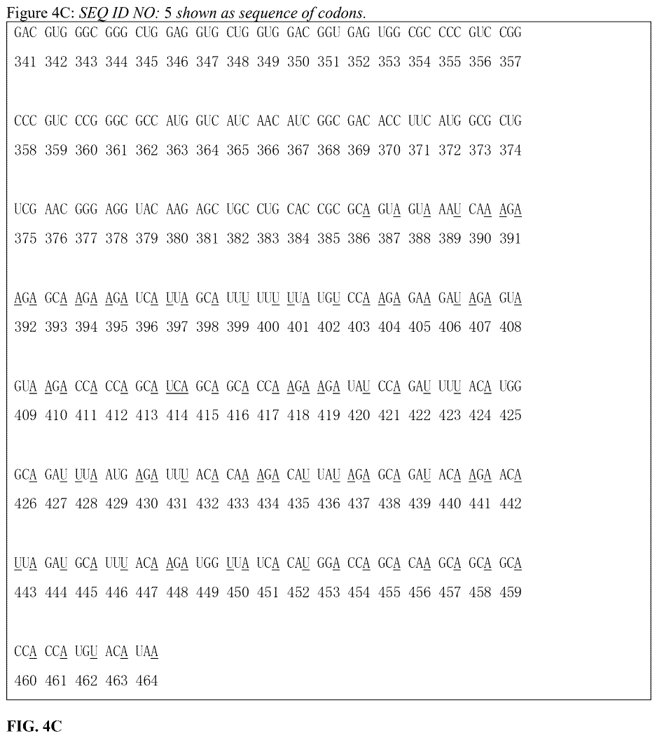

For convenience, FIG. 4 shows the ICS(PD₃₈₆₋) sequence with triplets numbered, differences underlined.

The implied coding sequence ICS(PD₃₈₄₋), with trained expression level 44.9%, is shown below with the difference from ICS(PD₃₈₆₋) underlined.

SEQ ID NO: 6 provides a design for a nucleic acid CDS sequence trained to a 44.9% expression
design level encoding of the Zm00001eb366090_P001 variant of a GA20ox3 protein of Zea mays
AUGAGGCCGCGCCUCCCUCCAAAUGUUCCCUCCCUGCCUUCGUCUUUGUCGUUGCUCGCAAACUCCCUGUCCUCCCCUGUUAC
AAAUACCCCCACCCGCCCGGACAGCUUCCCUGCAUACUUGCAGCUCGCACAUCUCAUGGUGUCGCAGGAACGACAAGAGCCAG
CUGUGCCUAGCAGCAGCAGCAGCAGCGCCAAGCGCGCAGCCACGUCCAUGGACGCCAGCCCGGCCCCGCCGCUCCUCCUCCGC
GCCCCCACUCCCAGCCCCAGCAUUGACCUCCCCGCUGGCAAGGACAAGGCCGACGCGGCGGCCAGCAAGGCCGGCGCGGCCGU
GUUCGACCUGCGCCGGGAGCCCAAGAUCCCCGCGCCAUUCCUGUGGCCGCAGGAAGAGGCGCGGCCGUCCUCGGCCGCGGAGC
UGGAGGUGCCGAUGGUGGACGUGGGCGUGCUGCGCAAUGGCGACCGCGCGGGGCUGCGGCGCGCCGCGGCGCAGGUGGCCGCG
GCGUGCGCGACGCACGGGUUCUUCCAGGUGUGCGGGCACGGCGUGGACGCGGCGCUGGGGCGCGCCGCGCUGGACGGCGCCAG
CGACUUCUUCCGGCUGCCGCUCGCCGAGAAGCAGCGCGCCCGGCGCGUCCCCGGCACCGUGUCCGGGUACACGAGCGCGCACG
CCGACCGGUUCGCGGCCAAGCUCCCCUGGAAGGAGACCCUGUCGUUCGGCUACCACGACGGCGCCGCGUCGCCUGUCGUCGUG
GACUACUUCGUCGGCACCCUCGGCCAGGAUUUCGAGCCAAUGGGGUGGGUGUACCAGAGGUACUGCGAGGAGAUGAAGGAGCU
GUCGCUGACGAUCAUGGAGCUGCUGGAGCUGAGCCUGGGCGUGGAGCUGCGCGGCUACUACCGGGAGUUCUUCGAGGACAGCC
GGUCCAUCAUGCGGUGCAACUACUACCCGCCGUGCCCGGAGCCGGAGCGCACGCUGGGCACGGGCCCGCACUGCGACCCCACG
GCGCUCACCAUCCUCCUGCAGGACGACGUGGGCGGGCUGGAGGUGCUGGUGGACGGUGAGUGGCGCCCCGUCCGGCCCGUCCC
GGGCGCCAUGGUCAUCAACAUCGGCGACACCUUCAUGGCGCUGUCGAACGGGAGGUACAAGAGCUGCCUGCACAGAGCAGUAG
UAAAUCAAAGAAGAGCAAGAAGAUCAUUAGCAUUUUUUUUAUGUCCAAGAGAAGAUAGAGUAGUAAGACCACCAGCAUCAGCA
GCACCAAGAAGAUAUCCAGAUUUUACAUGGGCAGAUUUAAUGAGAUUUACACAAAGACAUUAUAGAGCAGAUACAAGAACAUU
AGAUGCAUUUACAAGAUGGUUAUCACAUGGACCAGCACAAGCAGCAGCACCACCAUGUACAUAA

One skilled in the art shall recognize that one need not allow specific triplets or other subsequences of importance to change.

Example segment of GA20ox3 DNA of Zea Mays B73 inferred from the RNA (primary transcript) is shown below.

SEQ ID NO: 7 provides the polynucleotide sequence of the coding strand of the DNA region
transcribed then spliced to the Zm00001eb366090_T001 mRNA encoding of the
Zm00001eb366090_P001 variant of a GA20ox3 protein of Zea mays
TGTCGGGTCCCACATGAGGCCGCGCCTCCCTCCAAATGTTCCCTCCCTGCCTTCGTCTTTGTCGTTGCTCGCAAACTCCCTGT
CCTCCCCTGTTACAAATACCCCCACCCGCCCGGACAGCTTCCCTGCATACTTGCAGCTCGCACATCTCATGGTGTCGCAGGAA
CGACAAGAGCCAGCTGTGCCTAGCAGCAGCAGCAGCAGCGCCAAGCGCGCAGCCACGTCCATGGACGCCAGCCCGGCCCCGCC
GCTCCTCCTCCGCGCCCCCACTCCCAGCCCCAGCATTGACCTCCCCGCTGGCAAGGACAAGGCCGACGCGGCGGCCAGCAAGG
CCGGCGCGGCCGTGTTCGACCTGCGCCGGGAGCCCAAGATCCCCGCGCCATTCCTGTGGCCGCAGGAAGAGGCGCGGCCGTCC
TCGGCCGCGGAGCTGGAGGTGCCGATGGTGGACGTGGGCGTGCTGCGCAATGGCGACCGCGCGGGGCTGCGGCGCGCCGCGGC
GCAGGTGGCCGCGGCGTGCGCGACGCACGGGTTCTTCCAGGTGTGCGGGCACGGCGTGGACGCGGCGCTGGGGCGCGCCGCGC
TGGACGGCGCCAGCGACTTCTTCCGGCTGCCGCTCGCCGAGAAGCAGCGCGCCCGGCGCGTCCCCGGCACCGTGTCCGGGTAC
ACGAGCGCGCACGCCGACCGGTTCGCGGCCAAGCTCCCCTGGAAGGAGACCCTGTCGTTCGGCTACCACGACGGCGCCGCGTC
GCCTGTCGTCGTGGACTACTTCGTCGGCACCCTCGGCCAGGATTTCGAGCCAATGGGGTAAGTAAGGTAGTAAGAAGGAGCGC
CGGTTTACATTTACCGCACGTCGGCGTGCGGTCGAGTCGGGACTCGGGAGACGTATGAACCCCCGTCCCGTCCCATGCATGTG
TGGCAGGTGGGTGTACCAGAGGTACTGCGAGGAGATGAAGGAGCTGTCGCTGACGATCATGGAGCTGCTGGAGCTGAGCCTGG
GCGTGGAGCTGCGCGGCTACTACCGGGAGTTCTTCGAGGACAGCCGGTCCATCATGCGGTGCAACTACTACCCGCCGTGCCCG
GAGCCGGAGCGCACGCTGGGCACGGGCCCGCACTGCGACCCCACGGCGCTCACCATCCTCCTGCAGGACGACGTGGGCGGGCT
GGAGGTGCTGGTGGACGGTGAGTGGCGCCCCGTCCGGCCCGTCCCGGGCGCCATGGTCATCAACATCGGCGACACCTTCATGG
TAACGAAACGAAAGCGCTCGCTCCTCTGTTTTCCTTGGCCGCTCTTGTCCTGTGTGTATATTCAGTTGAGCTCTCTCTGTGCT
GTTATTTCCCGAATCCTAGTGGACCTAAACGGGCAGGTTATTACAGCACGCACACGTAGGCATGTCATGTAGCTAGTACATAC
ATAGCGATGCCGATGCAAATGCAATAGAGACATGCGTTCGAGTTGGTTCCTATCTCGGCGGGCTACGGCAGGTACACGCGGCC
GCGGCGCGCTCTCTCTAGTCTATCCGCGGCCGCGCCCAGGCCGATCGAGGCTTCCGGGGGAGAGTTGCGACAAGAGAACGGAC
CGAGGGGGTCGGCTAGCGGTAGCAAGTTCCCTGTTGGTTTGTGGCGTTGGAGCGTTGCGGAGAGGCTTGCGCGGCGGCGGGGA
CGTCGACGGGGACGTGGCGGGGAGACGATACGATGGGTGCCGGGCAGGGCAACGCTTTCGGCGGGTGGCCGTGTCCAGGTGCG
CGCGGCCTTGTCGGTTTCCCCCTCTCGGTGTCCATGGCCGAGAAATGGGTCGACGACCGAGACCGACGCTCGGTGCGGCGCCC
ATCCCGTCTGATCCGCCGCGCCACGCGAGCGGCCCTATGCGATGCCGCACGGGCGCGGAGGGCCGTCGCGCGGAGTATAATGT
ATAGTATATAGTACAAGGTTGGTTGGAGTCGGGTTGGGTTGGATCGGGTCACCGGTACGTGGTGGCTGCTGTTGCCCCCGCCG
TTTCCGCTTGCACTTTTGTCGCGGTTTCGCTGGCGATCCGGCACGCGGCGCCCACACCACGCCGGGGCTCCAAACAGCTCGGG
CCCTTGGCCGTGTGGGTGGCAGGCACTTGCACGCGTCCGGTTGTCGCGGCCTGGCCCGCCGCCGGGCGCACCGCAACAATGAG
ACAGCCCGACACGATGATTCTTGTGCACTGTGCTAACCCGCATGCCATGCAGGCGCTGTCGAACGGGAGGTACAAGAGCTGCC
TGCACCGCGCGGTGGTGAACCAGCGGCGGGCGCGGCGGTCGCTGGCCTTCTTCCTGTGCCCGCGCGAGGACCGGGTGGTGCGC
CCGCCGGCCAGTGCTGCGCCGCGGCGCTACCCGGACTTCACCTGGGCCGACCTCATGCGCTTCACGCAGCGCCACTACCGCGC
CGACACCCGCACGCTGGACGCCTTCACCCGCTGGCTCTCCCACGGCCCGGCCCAGGCGGCGGCGCCTCCCTGCACCTAGCGAG
CCGGGCCAAGGCCGTCTCTTTCGCCCCACGTGCGCGCCCAGCTGGGCAGGTGGCCAGACACGCGGCCCGCGGGCCCCGCGCCG
CCTTGCCATTTTTTGACGCTGGCCCTACTGCTGTGCTACTAGTGTACATATGCAAGAGTACATATATATATATATATATACGT
ATTTTCTATATATTATATATAAAAGCAAGGCGGCCCGGTGCCCTTCTCTTGTTTTGTCCACAACTGTTTGATCCCATTATTCT
ATGGACCATGGATACTTCAATGTTTGTACTAAGACCGTGAACGTGGGATTCTTTTCCTTCCTCTGTGTTTTTTCTGAGAAAAA
TTAAACTGATTTCTGTGAA

Given the B73 DNA and the CDS (i.e. the Reference RNA above), a mapping via an alignment between relevant section of DNA and the CDS sequences is shown via underlining in sequence below. The concatenation of the underlined subsequences forms the CDS.

TABLE 5
SEQ ID NO: 7
TGTCGGGTCCCACATGAGGCCGCGCCTCCCTCCAAATGTTCCCTCCCTGCCTTCGTCTTTGTCGTTGCTCGCAAACTCCCTGT
CCTCCCCTGTTACAAATACCCCCACCCGCCCGGACAGCTTCCCTGCATACTTGCAGCTCGCACATCTCATGGTGTCGCAGGAA
CGACAAGAGCCAGCTGTGCCTAGCAGCAGCAGCAGCAGCGCCAAGCGCGCAGCCACGTCCATGGACGCCAGCCCGGCCCCGCC
GCTCCTCCTCCGCGCCCCCACTCCCAGCCCCAGCATTGACCTCCCCGCTGGCAAGGACAAGGCCGACGCGGCGGCCAGCAAGG
CCGGCGCGGCCGTGTTCGACCTGCGCCGGGAGCCCAAGATCCCCGCGCCATTCCTGTGGCCGCAGGAAGAGGCGCGGCCGTCC
TCGGCCGCGGAGCTGGAGGTGCCGATGGTGGACGTGGGCGTGCTGCGCAATGGCGACCGCGCGGGGCTGCGGCGCGCCGCGGC
GCAGGTGGCCGCGGCGTGCGCGACGCACGGGTTCTTCCAGGTGTGCGGGCACGGCGTGGACGCGGCGCTGGGGCGCGCCGCGC
TGGACGGCGCCAGCGACTTCTTCCGGCTGCCGCTCGCCGAGAAGCAGCGCGCCCGGCGCGTCCCCGGCACCGTGTCCGGGTAC
ACGAGCGCGCACGCCGACCGGTTCGCGGCCAAGCTCCCCTGGAAGGAGACCCTGTCGTTCGGCTACCACGACGGCGCCGCGTC
GCCTGTCGTCGTGGACTACTTCGTCGGCACCCTCGGCCAGGATTTCGAGCCAATGGGGTAAGTAAGGTAGTAAGAAGGAGCGC
CGGTTTACATTTACCGCACGTCGGCGTGCGGTCGAGTCGGGACTCGGGAGACGTATGAACCCCCGTCCCGTCCCATGCATGTG
TGGCAGGTGGGTGTACCAGAGGTACTGCGAGGAGATGAAGGAGCTGTCGCTGACGATCATGGAGCTGCTGGAGCTGAGCCTGG
GCGTGGAGCTGCGCGGCTACTACCGGGAGTTCTTCGAGGACAGCCGGTCCATCATGCGGTGCAACTACTACCCGCCGTGCCCG
GAGCCGGAGCGCACGCTGGGCACGGGCCCGCACTGCGACCCCACGGCGCTCACCATCCTCCTGCAGGACGACGTGGGCGGGCT
GGAGGTGCTGGTGGACGGTGAGTGGCGCCCCGTCCGGCCCGTCCCGGGCGCCATGGTCATCAACATCGGCGACACCTTCATGG
TAACGAAACGAAAGCGCTCGCTCCTCTGTTTTCCTTGGCCGCTCTTGTCCTGTGTGTATATTCAGTTGAGCTCTCTCTGTGCT
GTTATTTCCCGAATCCTAGTGGACCTAAACGGGCAGGTTATTACAGCACGCACACGTAGGCATGTCATGTAGCTAGTACATAC
ATAGCGATGCCGATGCAAATGCAATAGAGACATGCGTTCGAGTTGGTTCCTATCTCGGCGGGCTACGGCAGGTACACGCGGCC
GCGGCGCGCTCTCTCTAGTCTATCCGCGGCCGCGCCCAGGCCGATCGAGGCTTCCGGGGGAGAGTTGCGACAAGAGAACGGAC
CGAGGGGGTCGGCTAGCGGTAGCAAGTTCCCTGTTGGTTTGTGGCGTTGGAGCGTTGCGGAGAGGCTTGCGCGGCGGCGGGGA
CGTCGACGGGGACGTGGCGGGGAGACGATACGATGGGTGCCGGGCAGGGCAACGCTTTCGGCGGGTGGCCGTGTCCAGGTGCG
CGCGGCCTTGTCGGTTTCCCCCTCTCGGTGTCCATGGCCGAGAAATGGGTCGACGACCGAGACCGACGCTCGGTGCGGCGCCC
ATCCCGTCTGATCCGCCGCGCCACGCGAGCGGCCCTATGCGATGCCGCACGGGCGCGGAGGGCCGTCGCGCGGAGTATAATGT
ATAGTATATAGTACAAGGTTGGTTGGAGTCGGGTTGGGTTGGATCGGGTCACCGGTACGTGGTGGCTGCTGTTGCCCCCGCCG
TTTCCGCTTGCACTTTTGTCGCGGTTTCGCTGGCGATCCGGCACGCGGCGCCCACACCACGCCGGGGCTCCAAACAGCTCGGG
CCCTTGGCCGTGTGGGTGGCAGGCACTTGCACGCGTCCGGTTGTCGCGGCCTGGCCCGCCGCCGGGCGCACCGCAACAATGAG
ACAGCCCGACACGATGATTCTTGTGCACTGTGCTAACCCGCATGCCATGCAGGCGCTGTCGAACGGGAGGTACAAGAGCTGCC
TGCACCGCGCGGTGGTGAACCAGCGGCGGGCGCGGCGGTCGCTGGCCTTCTTCCTGTGCCCGCGCGAGGACCGGGTGGTGCGC
CCGCCGGCCAGTGCTGCGCCGCGGCGCTACCCGGACTTCACCTGGGCCGACCTCATGCGCTTCACGCAGCGCCACTACCGCGC
CGACACCCGCACGCTGGACGCCTTCACCCGCTGGCTCTCCCACGGCCCGGCCCAGGCGGCGGCGCCTCCCTGCACCTAGCGAG
CCGGGCCAAGGCCGTCTCTTTCGCCCCACGTGCGCGCCCAGCTGGGCAGGTGGCCAGACACGCGGCCCGCGGGCCCCGCGCCG
CCTTGCCATTTTTTGACGCTGGCCCTACTGCTGTGCTACTAGTGTACATATGCAAGAGTACATATATATATATATATATACGT
ATTTTCTATATATTATATATAAAAGCAAGGCGGCCCGGTGCCCTTCTCTTGTTTTGTCCACAACTGTTTGATCCCATTATTCT
ATGGACCATGGATACTTCAATGTTTGTACTAAGACCGTGAACGTGGGATTCTTTTCCTTCCTCTGTGTTTTTTCTGAGAAAAA
TTAAACTGATTTCTGTGAA

Mapping the difference of ICS(PD₃₈₆₋) and the reference RNA sequence onto the B73 DNA that encodes the primary transcript, gives a new GA20ox3 DNA sequence with CDS underlined and the differences highlighted in bold with wavy underline

provides a design for a polynucleotide sequence to help identify what needs DNA
editing with CDS trained to a 44.9% expression design level encoding of the
Zm00001eb366090_P001 variant of a GA200x3 protein of Zea mays
SEQ ID NO: 8
TGTCGGGTCCCACATGAGGCCGCGCCTCCCTCCAAATGTTCCCTCCCTGCCTTCGTCTTTGTCGTTGCTCGCAAACTCCCTGT
CCTCCCCTGTTACAAATACCCCCACCCGCCCGGACAGCTTCCCTGCATACTTGCAGCTCGCACATCTCATGGTGTCGCAGGAA
CGACAAGAGCCAGCTGTGCCTAGCAGCAGCAGCAGCAGCGCCAAGCGCGCAGCCACGTCCATGGACGCCAGCCCGGCCCCGCC
GCTCCTCCTCCGCGCCCCCACTCCCAGCCCCAGCATTGACCTCCCCGCTGGCAAGGACAAGGCCGACGCGGCGGCCAGCAAGG
CCGGCGCGGCCGTGTTCGACCTGCGCCGGGAGCCCAAGATCCCCGCGCCATTCCTGTGGCCGCAGGAAGAGGCGCGGCCGTCC
TCGGCCGCGGAGCTGGAGGTGCCGATGGTGGACGTGGGCGTGCTGCGCAATGGCGACCGCGCGGGGCTGCGGCGCGCCGCGGC
GCAGGTGGCCGCGGCGTGCGCGACGCACGGGTTCTTCCAGGTGTGCGGGCACGGCGTGGACGCGGCGCTGGGGCGCGCCGCGC
TGGACGGCGCCAGCGACTTCTTCCGGCTGCCGCTCGCCGAGAAGCAGCGCGCCCGGCGCGTCCCCGGCACCGTGTCCGGGTAC
ACGAGCGCGCACGCCGACCGGTTCGCGGCCAAGCTCCCCTGGAAGGAGACCCTGTCGTTCGGCTACCACGACGGCGCCGCGTC
GCCTGTCGTCGTGGACTACTTCGTCGGCACCCTCGGCCAGGATTTCGAGCCAATGGGGTAAGTAAGGTAGTAAGAAGGAGCGC
CGGTTTACATTTACCGCACGTCGGCGTGCGGTCGAGTCGGGACTCGGGAGACGTATGAACCCCCGTCCCGTCCCATGCATGTG
TGGCAGGTGGGTGTACCAGAGGTACTGCGAGGAGATGAAGGAGCTGTCGCTGACGATCATGGAGCTGCTGGAGCTGAGCCTGG
GCGTGGAGCTGCGCGGCTACTACCGGGAGTTCTTCGAGGACAGCCGGTCCATCATGCGGTGCAACTACTACCCGCCGTGCCCG
GAGCCGGAGCGCACGCTGGGCACGGGCCCGCACTGCGACCCCACGGCGCTCACCATCCTCCTGCAGGACGACGTGGGCGGGCT
GGAGGTGCTGGTGGACGGTGAGTGGCGCCCCGTCCGGCCCGTCCCGGGCGCCATGGTCATCAACATCGGCGACACCTTCATGG
TAACGAAACGAAAGCGCTCGCTCCTCTGTTTTCCTTGGCCGCTCTTGTCCTGTGTGTATATTCAGTTGAGCTCTCTCTGTGCT
GTTATTTCCCGAATCCTAGTGGACCTAAACGGGCAGGTTATTACAGCACGCACACGTAGGCATGTCATGTAGCTAGTACATAC
ATAGCGATGCCGATGCAAATGCAATAGAGACATGCGTTCGAGTTGGTTCCTATCTCGGGGGCTACGGCAGGTACACGCGGCC
GCGGCGCGCTCTCTCTAGTCTATCCGCGGCCGCGCCCAGGCCGATCGAGGCTTCCGGGGGAGAGTTGCGACAAGAGAACGGAC
CGAGGGGGTCGGCTAGCGGTAGCAAGTTCCCTGTTGGTTTGTGGCGTTGGAGCGTTGCGGAGAGGCTTGCGCGGCGGCGGGGA
CGTCGACGGGGACGTGGCGGGGAGACGATACGATGGGTGCCGGGCAGGGCAACGCTTTCGGCGGGTGGCCGTGTCCAGGTGCG
CGCGGCCTTGTCGGTTTCCCCCTCTCGGTGTCCATGGCCGAGAAATGGGTCGACGACCGAGACCGACGCTCGGTGCGGCGCCC
ATCCCGTCTGATCCGCCGCGCCACGCGAGCGGCCCTATGCGATGCCGCACGGGCGCGGAGGGCCGTCGCGCGGAGTATAATGT
ATAGTATATAGTACAAGGTTGGTTGGAGTCGGGTTGGGTTGGATCGGGTCACCGGTACGTGGTGGCTGCTGTTGCCCCCGCCG
TTTCCGCTTGCACTTTTGTCGCGGTTTCGCTGGCGATCCGGCACGCGGCGCCCACACCACGCCGGGGCTCCAAACAGCTCGGG
CCCTTGGCCGTGTGGGTGGCAGGCACTTGCACGCGTCCGGTTGTCGCGGCCTGGCCCGCCGCCGGGCGCACCGCAACAATGAG
ACAGCCCGACACGATGATTCTTGTGCACTGTGCTAACCCGCATGCCATGCAGGCGCTGTCGAACGGGAGGTACAAGAGCTGCC

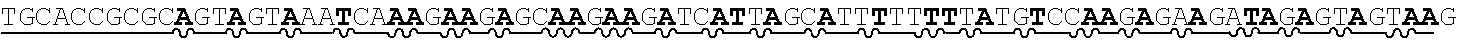

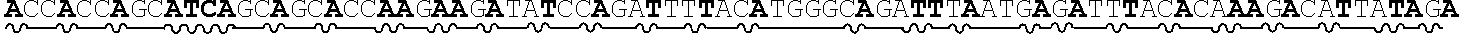

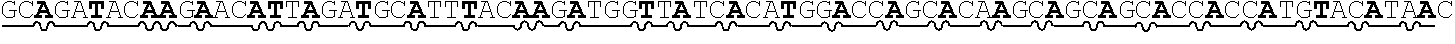

GAGCCGGGCCAAGGCCGTCTCTTTCGCCCCACGTGCGCGCCCAGCTGGGCAGGTGGCCAGACACGCGGCCCGCGGGCCCCGCG
CCGCCTTGCCATTTTTTGACGCTGGCCCTACTGCTGTGCTACTAGTGTACATATGCAAGAGTACATATATATATATATATATA
CGTATTTTCTATATATTATATATAAAAGCAAGGCGGCCCGGTGCCCTTCTCTTGTTTTGTCCACAACTGTTTGATCCCATTAT
TCTATGGACCATGGATACTTCAATGTTTGTACTAAGACCGTGAACGTGGGATTCTTTTCCTTCCTCTGTGTTTTTTCTGAGAA
AAATTAAACTGATTTCTGTGAA

Alternatively we may increase the expression of Zm00001eb366090_P₀₀₁ by increasing the expression design level towards increasing floral traits such as ear row number and maize yield when damaging wind events are not expected.

### B. Example 2: Gene Training to Dial in Expression of a Transgene

In this example, expression of a protein is trained to generate a polynucleotide sequence to a specified relative expression design level without starting from the reference transcript. One may choose to not leverage an existing transcript or its CDS when it is convenient, for example when one wishes to replace or add a gene.

In this example, we train a protein sequence without leveraging a reference transcript. Instead, we begin only with a protein sequence and a set of candidate relative expression design levels, to use in due experimentation towards determining a safe and effective level of an insect toxin to impart insect resistance to a crop. In this example we use the Cry3Bb1 protein sequence, an insect toxin for Coleopteran such as Western Corn Rootworm, which by way of methods of this disclosure, can be used to increase durability of the Cry3Bb1 trait to counteract some types of gradually acquired resistance by Coleopteran such as Western Corn Rootworm.

Example Protein Sequence (Cry3Bb1 protein)

SEQ ID NO: 9 provides the amino acid sequence of a variant of Cry3Bbl protein from
U.S. Pat. No. 7,705,216
MANPNNRSEHDTIKVTPNSELQTNHNQYPLADNPNSTLEELNYKEFLRMTEDSSTEVLDNSTVKDAVGTGISVVGQILGVVGV
PFAGALTSFYQSFLNTIWPSDADPWKAFMAQVEVLIDKKIEEYAKSKALAELQGLQNNFEDYVNALNSWKKTPLSLRSKRSQG
RIRELFSQAESHFRNSMPSFAVSKFEVLFLPTYAQAANTHLLLLKDAQVFGEEWGYSSEDVAEFYRRQLKLTQQYTDHCVNWY
NVGLNGLRGSTYDAWVKFNRFRREMTLTVLDLIVLFPFYDIRLYSKGVKTELTRDIFTDPIFLLTTLQKYGPTFLSIENSIRK
PHLFDYLQGIEFHTRLRPGYFGKDSFNYWSGNYVETRPSIGSSKTITSPFYGDKSTEPVQKLSFDGQKVYRTIANTDVAAWPN
GKVYLGVTKVDFSQYDDQKNETSTQTYDSKRNNGHVSAQDSIDQLPPETTDEPLEKAYSHQLNYAECFLMQDRRGTIPFFTWT
HRSVDFFNTIDAEKITQLPVVKAYALSSGASIIEGPGFTGGNLLFLKESSNSIAKFKVTLNSAALLQRYRVRIRYASTTNLRL
FVQNSNNDFLVIYINKTMNKDDDLTYQTFDLATTNSNMGFSGDKNELIIGAESFVSNEKIYIDKIEFIPVQL

Upper Calibrating Cry3Bb1 CDS RNA Sequence (For Calibration via CDSFold, −MFE=1248; [0-1] design level=100%):

SEQ ID NO: 10 provides a design for a nucleic acid CDS sequence trained to a 44.9%
expression design level encoding of the Zm00001eb366090_P001 variant of a GA200x3 protein
of Zea mays

AUGGCGAAUCCGAACAACCGGUCUGAGCAUGAUACGAUCAAAGUGACUCCGAACUCGGAGUUACAGACGAAUCAUAAUCAGUA
CCCGUUGGCGGAUAAUCCCAACAGCACACUGGAAGAACUCAAUUAUAAGGAGUUCUUACGAAUGACCGAAGAUUCGUCGACCG
AGGUCCUGGAUAACUCCACCGUGAAAGACGCGGUGGGGACCGGGAUCUCGGUUGUCGGUCAGAUCCUCGGCGUGGUCGGAGUG
CCUUUCGCGGGUGCGCUAACGUCGUUCUACCAGUCGUUUUUAAAUACGAUCUGGCCGAGCGACGCGGACCCGUGGAAGGCAUU
CAUGGCCCAGGUCGAGGUUCUGAUCGACAAGAAGAUAGAGGAGUAUGCCAAGAGCAAGGCGUUGGCGGAGCUUCAAGGUUUAC
AAAAUAACUUUGAAGACUACGUCAACGCCUUGAACUCUUGGAAAAAGACUCCUCUAUCUUUGCGGAGUAAGCGCAGUCAGGGC
CGGAUCCGUGAGCUCUUCUCCCAGGCGGAAUCGCAUUUUCGGAACUCGAUGCCAAGCUUCGCUGUGUCGAAGUUCGAAGUCCU
GUUCUUGCCCACGUAUGCCCAAGCGGCGAAUACCCACCUACUCCUCCUCAAAGACGCACAGGUCUUUGGGGAGGAGUGGGGGU
AUUCGUCGGAGGAUGUCGCCGAGUUCUAUCGGCGACAACUCAAGUUGACGCAGCAGUACACAGACCACUGCGUCAACUGGUAU
AACGUGGGCUUGAACGGACUUCGAGGUUCGACAUACGAUGCUUGGGUCAAGUUCAAUCGAUUCCGCCGGGAGAUGACGCUCAC
GGUUCUGGACCUGAUUGUGCUUUUUCCGUUUUACGACAUCCGUCUUUACUCCAAAGGAGUGAAGACGGAGUUGACUCGCGAUA
UCUUUACGGACCCGAUAUUUCUGCUAACAACGCUGCAGAAAUAUGGGCCGACUUUCUUAUCGAUUGAAAAUUCGAUAAGAAAG
CCCCAUUUGUUCGACUACCUUCAGGGCAUCGAGUUCCACACCCGGUUGAGGCCCGGGUACUUCGGUAAAGAUAGCUUCAACUA
CUGGUCGGGGAACUACGUGGAAACGAGGCCCUCGAUCGGUAGUUCGAAGACUAUCACGUCUCCGUUUUACGGCGACAAGUCGA
CGGAGCCUGUGCAGAAGCUAUCGUUCGAUGGACAAAAAGUCUAUCGAACGAUAGCUAAUACUGACGUGGCCGCUUGGCCGAAU
GGAAAGGUAUAUCUGGGUGUGACGAAAGUCGACUUCAGUCAGUACGAUGACCAGAAGAACGAGACGUCCACCCAGACGUACGA
UUCCAAGCGGAACAACGGCCACGUCUCUGCACAGGACUCCAUCGACCAGUUGCCGCCGGAGACGACGGACGAGCCGCUGGAGA
AGGCGUACUCUCACCAGCUGAAUUAUGCUGAGUGUUUUCUCAUGCAGGAUAGGAGGGGGACAAUCCCCUUCUUCACCUGGACG
CAUCGAUCGGUUGAUUUUUUCAACACGAUCGAUGCGGAGAAGAUCACUCAGCUUCCGGUGGUGAAGGCGUACGCCUUGUCCAG
CGGCGCGUCCAUUAUCGAAGGCCCGGGCUUCACCGGGGGGAACUUGUUGUUCCUGAAGGAGUCGAGCAACAGCAUCGCCAAAU
UUAAGGUGACGCUGAAUUCUGCAGCGUUGUUGCAGAGAUAUCGGGUCCGUAUAAGAUACGCGAGUACGACGAAUCUUCGGUUA
UUCGUGCAGAACUCCAAUAAUGAUUUUCUCGUUAUUUACAUUAAUAAAACUAUGAAUAAGGACGACGACCUCACUUAUCAAAC
GUUCGAUCUUGCCACCACGAACUCCAACAUGGGGUUCAGUGGUGACAAGAACGAACUGAUAAUUGGGGCGGAGUCCUUUGUGA
GUAACGAGAAGAUCUAUAUUGAUAAGAUUGAGUUCAUUCCAGUGCAGCUGUAA

Lower Calibrating Cry3Bb1 CDS RNA Sequence (For Calibration via CDSFold, −MFE=222; [0-1] design level-0%):

SEQ ID NO: 11 provides a design for a nucleic acid CDS sequence trained to a 0% expression
design level encoding of a Cry3Bbl protein variant from US7705216

AUGGCAAACCCAAACAACAGAUCAGAACACGACACAAUAAAAGUAACACCAAACUCAGAACUACAAACAAACCACAACCAAUA
CCCACUAGCAGACAACCCAAACUCAACACUAGAAGAACUAAACUACAAAGAAUUCCUAAGAAUGACAGAAGACUCAUCAACAG
AAGUACUAGACAACUCAACAGUAAAAGACGCAGUAGGAACAGGAAUAUCAGUAGUAGGACAAAUACUAGGAGUAGUAGGAGUA
CCAUUCGCAGGAGCACUAACAUCAUUCUACCAAUCAUUCCUAAACACAAUAUGGCCAUCAGACGCAGACCCAUGGAAAGCAUU
CAUGGCACAAGUAGAAGUACUAAUAGACAAAAAAAUAGAAGAAUACGCAAAAUCAAAAGCACUAGCAGAACUACAAGGACUAC
AAAACAACUUCGAAGACUACGUAAACGCACUAAACUCAUGGAAAAAAACACCACUAUCACUAAGAUCAAAAAGAUCACAAGGA
AGAAUAAGAGAACUAUUCUCACAAGCAGAAUCACACUUCAGAAACUCAAUGCCAUCAUUCGCAGUAUCAAAAUUCGAAGUACU
AUUCCUACCAACAUACGCACAAGCAGCAAACACACACCUACUACUACUAAAAGACGCACAAGUAUUCGGAGAAGAAUGGGGAU
ACUCAUCAGAAGACGUAGCAGAAUUCUACAGAAGACAACUAAAACUAACACAACAAUACACAGACCACUGCGUAAACUGGUAC
AACGUAGGACUAAACGGACUAAGAGGAUCAACAUACGACGCAUGGGUAAAAUUCAACAGAUUCAGAAGAGAAAUGACACUAAC
AGUACUAGACCUAAUAGUACUAUUCCCAUUCUACGACAUAAGACUAUACUCAAAAGGAGUAAAAACAGAACUAACAAGAGACA
UAUUCACAGACCCAAUAUUCCUACUAACAACACUACAAAAAUACGGACCAACAUUCCUAUCAAUAGAAAACUCAAUAAGAAAA
CCACACCUAUUCGACUACCUACAAGGAAUAGAAUUCCACACAAGACUAAGACCAGGAUACUUCGGAAAAGACUCAUUCAACUA
CUGGUCAGGAAACUACGUAGAAACAAGACCAUCAAUAGGAUCAUCAAAAACAAUAACAUCACCAUUCUACGGAGACAAAUCAA
CAGAACCAGUACAAAAACUAUCAUUCGACGGACAAAAAGUAUACAGAACAAUAGCAAACACAGACGUAGCAGCAUGGCCAAAC
GGAAAAGUAUACCUAGGAGUAACAAAAGUAGACUUCUCACAAUACGACGACCAAAAAAACGAAACAUCAACACAAACAUACGA
CUCAAAAAGAAACAACGGACACGUAUCAGCACAAGACUCAAUAGACCAACUACCACCAGAAACAACAGACGAACCACUAGAAA
AAGCAUACUCACACCAACUAAACUACGCAGAAUGCUUCCUAAUGCAAGACAGAAGAGGAACAAUACCAUUCUUCACAUGGACA
CACAGAUCAGUAGACUUCUUCAACACAAUAGACGCAGAAAAAAUAACACAACUACCAGUAGUAAAAGCAUACGCACUAUCAUC
AGGAGCAUCAAUAAUAGAAGGACCAGGAUUCACAGGAGGAAACCUACUAUUCCUAAAAGAAUCAUCAAACUCAAUAGCAAAAU
UCAAAGUAACACUAAACUCAGCAGCACUACUACAAAGAUACAGAGUAAGAAUAAGAUACGCAUCAACAACAAACCUAAGACUA
UUCGUACAAAACUCAAACAACGACUUCCUAGUAAUAUACAUAAACAAAACAAUGAACAAAGACGACGACCUAACAUACCAAAC
AUUCGACCUAGCAACAACAAACUCAAACAUGGGAUUCUCAGGAGACAAAAACGAACUAAUAAUAGGAGCAGAAUCAUUCGUAU
CAAACGAAAAAAUAUACAUAGACAAAAUAGAAUUCAUACCAGUACAACUAUAA

Then, using the lower calibrating CDS RNA sequence as the primary bounding sequence and the upper calibrating CDS RNA sequence as the ultimate bounding sequence, the PBS is at 0% and the UBS is at 100%, with our desire to create a panel of relative expression design levels of 60%, 50%, and 40% for evaluation in a set of insect toxin assays. The set of differences from the PBS to the UBS are the triplet indices shown below. In this example, we use the contiguous ordering of the differences.

2 3 4 7 8 9 10 11 12 13 15 16 17 19 20 21 22 23 24 25 26 27 29 30 31 32 33 34 36 38
41 42 43 44 45 47 48 50 52 53 54 55 56 57 58 59 61 62 63 66 67 68 69 70 71 72 73 74
75 76 77 78 79 80 81 83 84 86 87 88 90 91 94 95 96 97 98 99 100 102 103 105 107 109
113 114 115 116 117 118 119 121 122 124 125 126 127 128 129 130 131 132 133 134 135
137 138 140 142 146 148 149 151 154 155 156 158 159 160 161 162 163 164 165 166 167
168 169 170 171 173 174 175 177 178 179 180 182 185 187 188 189 190 193 194 196 197
198 199 200 202 203 204 205 208 209 210 214 215 216 217 218 219 221 222 223 224 225
226 227 228 229 231 232 233 235 236 237 238 239 240 246 249 251 252 253 256 257 258
259 262 263 265 266 268 269 271 272 273 275 276 277 278 279 281 282 283 284 285 286
287 290 291 292 294 297 298 299 300 301 302 303 304 305 306 307 309 311 312 315 316
317 319 320 321 322 324 325 326 328 329 332 333 334 335 339 340 341 342 343 346 347
348 349 350 351 354 356 357 362 363 366 368 369 370 371 372 373 374 375 376 377 378
379 380 381 382 384 386 387 388 389 390 391 392 393 395 397 401 402 403 404 406 407
408 410 411 412 414 415 417 419 420 421 422 423 425 428 429 431 433 434 436 437 438
439 440 441 443 444 445 446 449 451 452 454 456 457 459 460 461 462 463 464 465 467
468 469 470 471 472 474 476 477 478 479 480 481 482 483 484 486 487 488 489 490 492
493 496 498 499 500 501 502 503 504 507 508 509 510 511 512 513 514 515 516 517 518
519 520 521 523 524 525 526 527 528 529 530 531 533 534 535 537 538 539 541 542 544
545 546 547 548 550 551 552 554 555 556 557 558 559 560 562 563 564 565 567 568 569
570 574 575 576 577 578 579 580 581 583 584 586 587 588 589 590 591 592 593 595 596
598 600 601 605 606 607 609 611 612 613 614 615 617 620 622 623 625 628 630 631 632
633 634 635 636 637 639 640 641 642 643 644 645 646 647 649 651 652 653

Given the difference above, an example of a (ascending contiguous) partial difference PD₂₈₁ can be described by the triplet indices numbered greater than or equal to 281. Then three narrow windows corresponding to the panel of relative expression design levels of 60%, 50%, and 40% (using −MFE as an example estimator and example ascending contiguous partial differences) are (PD₄₄₉,PD₄₅₁), (PD₃₇₄,PD₃₇₅), (PD₂₈₁,PD₂₈₂) with the implied coding sequences ICS(PD₄₅₁), ICS(PD₃₇₅), and ICS(PD₂₈₂) as follows:

ICS(PD₄₅₁) is an example Cry3Bb1 protein CDS RNA at design level ~ 60%, underlining representing differences from the reference:

(example is using −MFE=842 from RNAFold as the estimator)

SEQ ID NO: 12 provides a design for a nucleic acid CDS sequence trained to a 60% expression
design level encoding of a Cry3Bb1 protein variant from US7705216

AUGGCGAAUCCGAACAACCGGUCUGAGCAUGAUACGAUCAAAGUGACUCCGAACUCGGAGUUACAGACGAAUCAUAAUCAGUA
CCCGUUGGCGGAUAAUCCCAACAGCACACUGGAAGAACUCAAUUAUAAGGAGUUCUUACGAAUGACCGAAGAUUCGUCGACCG
AGGUCCUGGAUAACUCCACCGUGAAAGACGCGGUGGGGACCGGGAUCUCGGUUGUCGGUCAGAUCCUCGGCGUGGUCGGAGUG
CCUUUCGCGGGUGCGCUAACGUCGUUCUACCAGUCGUUUUUAAAUACGAUCUGGCCGAGCGACGCGGACCCGUGGAAGGCAUU
CAUGGCCCAGGUCGAGGUUCUGAUCGACAAGAAGAUAGAGGAGUAUGCCAAGAGCAAGGCGUUGGCGGAGCUUCAAGGUUUAC
AAAAUAACUUUGAAGACUACGUCAACGCCUUGAACUCUUGGAAAAAGACUCCUCUAUCUUUGCGGAGUAAGCGCAGUCAGGGC
CGGAUCCGUGAGCUCUUCUCCCAGGCGGAAUCGCAUUUUCGGAACUCGAUGCCAAGCUUCGCUGUGUCGAAGUUCGAAGUCCU
GUUCUUGCCCACGUAUGCCCAAGCGGCGAAUACCCACCUACUCCUCCUCAAAGACGCACAGGUCUUUGGGGAGGAGUGGGGGU
AUUCGUCGGAGGAUGUCGCCGAGUUCUAUCGGCGACAACUCAAGUUGACGCAGCAGUACACAGACCACUGCGUCAACUGGUAU
AACGUGGGCUUGAACGGACUUCGAGGUUCGACAUACGAUGCUUGGGUCAAGUUCAAUCGAUUCCGCCGGGAGAUGACGCUCAC
GGUUCUGGACCUGAUUGUGCUUUUUCCGUUUUACGACAUCCGUCUUUACUCCAAAGGAGUGAAGACGGAGUUGACUCGCGAUA
UCUUUACGGACCCGAUAUUUCUGCUAACAACGCUGCAGAAAUAUGGGCCGACUUUCUUAUCGAUUGAAAAUUCGAUAAGAAAG
CCCCAUUUGUUCGACUACCUUCAGGGCAUCGAGUUCCACACCCGGUUGAGGCCCGGGUACUUCGGUAAAGAUAGCUUCAACUA
CUGGUCGGGGAACUACGUGGAAACGAGGCCCUCGAUCGGUAGUUCGAAGACUAUCACGUCUCCGUUUUACGGCGACAAGUCGA
CGGAGCCUGUGCAGAAGCUAUCGUUCGAUGGACAAAAAGUCUAUCGAACGAUAGCUAAUACUGACGUGGCCGCUUGGCCGAAU
GGAAAGGUAUAUCUGGGUGUGACGAAAGUCGACUUCAGUCAGUACGAUGACCAGAAGAACGAGACGUCCACCCAGACGUACGA
UUCCAAGCGGAACAACGGCCACGUAUCAGCACAAGACUCAAUAGACCAACUACCACCAGAAACAACAGACGAACCACUAGAAA
AAGCAUACUCACACCAACUAAACUACGCAGAAUGCUUCCUAAUGCAAGACAGAAGAGGAACAAUACCAUUCUUCACAUGGACA
CACAGAUCAGUAGACUUCUUCAACACAAUAGACGCAGAAAAAAUAACACAACUACCAGUAGUAAAAGCAUACGCACUAUCAUC
AGGAGCAUCAAUAAUAGAAGGACCAGGAUUCACAGGAGGAAACCUACUAUUCCUAAAAGAAUCAUCAAACUCAAUAGCAAAAU
UCAAAGUAACACUAAACUCAGCAGCACUACUACAAAGAUACAGAGUAAGAAUAAGAUACGCAUCAACAACAAACCUAAGACUA
UUCGUACAAAACUCAAACAACGACUUCCUAGUAAUAUACAUAAACAAAACAAUGAACAAAGACGACGACCUAACAUACCAAAC
AUUCGACCUAGCAACAACAAACUCAAACAUGGGAUUCUCAGGAGACAAAAACGAACUAAUAAUAGGAGCAGAAUCAUUCGUAU
CAAACGAAAAAAUAUACAUAGACAAAAUAGAAUUCAUACCAGUACAACUAUAA

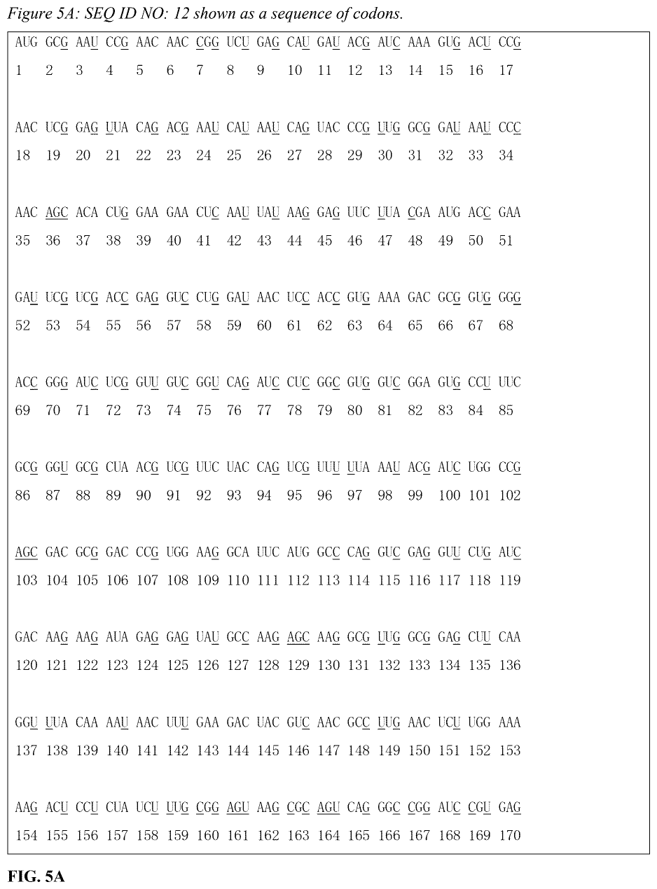

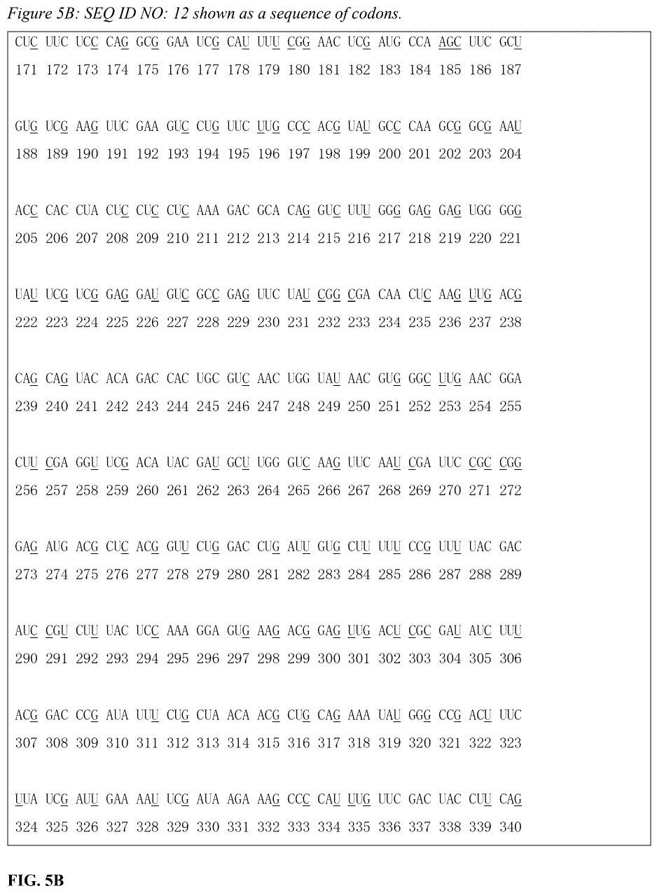

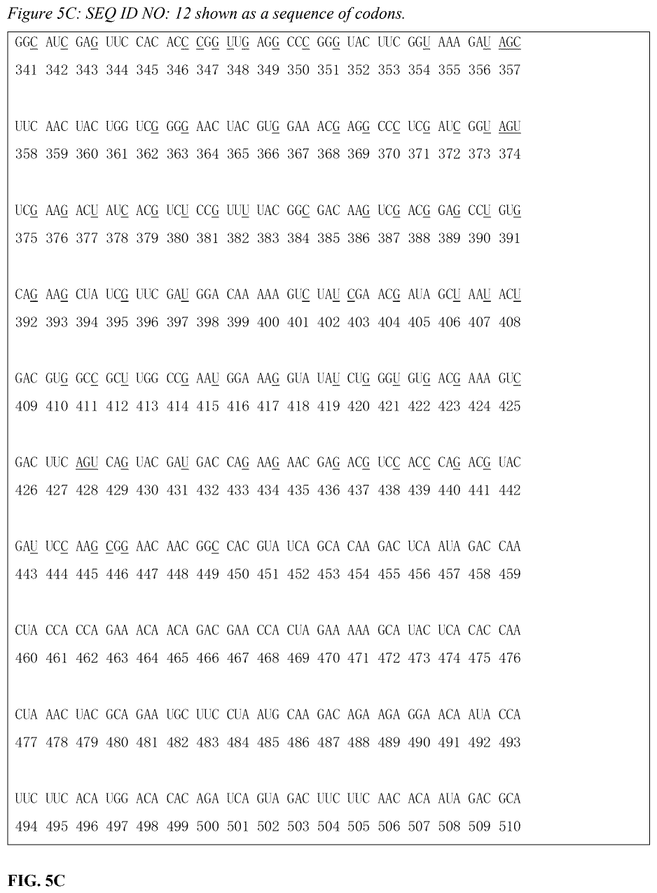

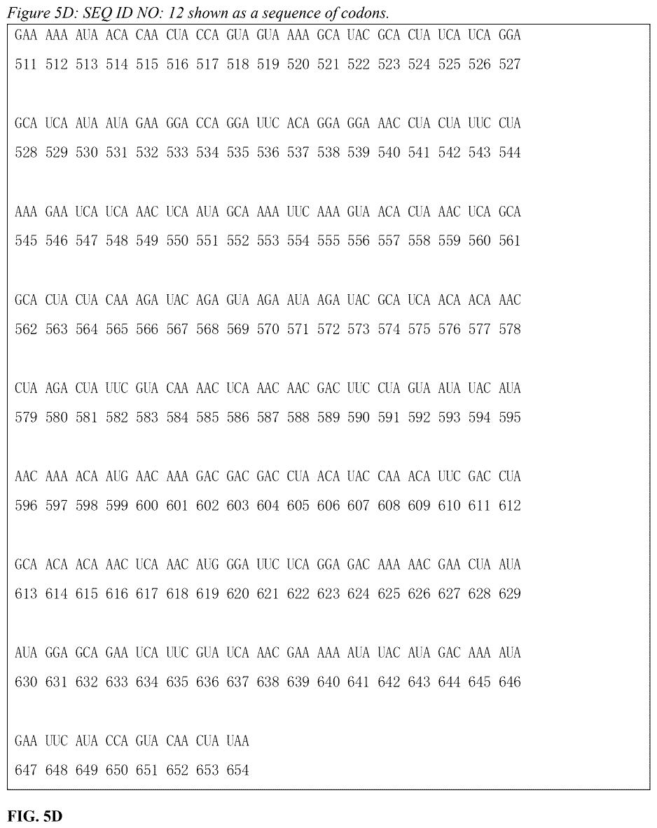

For convenience, FIG. 5 shows the ICS(PD₄₅₁) sequence SEQ ID NO: 12 with triplets numbered, with differences from reference underlined.

ICS(PD₃₇₅) is an example Cry3Bb1 protein CDS RNA at design level ~ 50%, with differences from reference underlined:

(example is using −MFE=735 from RNAFold as the estimator)

SEQ ID NO: 13 provides a design for a nucleic acid CDS sequence trained to a
50% expression design level encoding of a Cry3Bb1 protein variant from US7705216

AUGGCGAAUCCGAACAACCGGUCUGAGCAUGAUACGAUCAAAGUGACUCCGAACUCGGAGUUACAGACGAAUCAUAAUCAGUA
CCCGUUGGCGGAUAAUCCCAACAGCACACUGGAAGAACUCAAUUAUAAGGAGUUCUUACGAAUGACCGAAGAUUCGUCGACCG
AGGUCCUGGAUAACUCCACCGUGAAAGACGCGGUGGGGACCGGGAUCUCGGUUGUCGGUCAGAUCCUCGGCGUGGUCGGAGUG
CCUUUCGCGGGUGCGCUAACGUCGUUCUACCAGUCGUUUUUAAAUACGAUCUGGCCGAGCGACGCGGACCCGUGGAAGGCAUU
CAUGGCCCAGGUCGAGGUUCUGAUCGACAAGAAGAUAGAGGAGUAUGCCAAGAGCAAGGCGUUGGCGGAGCUUCAAGGUUUAC
AAAAUAACUUUGAAGACUACGUCAACGCCUUGAACUCUUGGAAAAAGACUCCUCUAUCUUUGCGGAGUAAGCGCAGUCAGGGC
CGGAUCCGUGAGCUCUUCUCCCAGGCGGAAUCGCAUUUUCGGAACUCGAUGCCAAGCUUCGCUGUGUCGAAGUUCGAAGUCCU
GUUCUUGCCCACGUAUGCCCAAGCGGCGAAUACCCACCUACUCCUCCUCAAAGACGCACAGGUCUUUGGGGAGGAGUGGGGGU
AUUCGUCGGAGGAUGUCGCCGAGUUCUAUCGGCGACAACUCAAGUUGACGCAGCAGUACACAGACCACUGCGUCAACUGGUAU
AACGUGGGCUUGAACGGACUUCGAGGUUCGACAUACGAUGCUUGGGUCAAGUUCAAUCGAUUCCGCCGGGAGAUGACGCUCAC
GGUUCUGGACCUGAUUGUGCUUUUUCCGUUUUACGACAUCCGUCUUUACUCCAAAGGAGUGAAGACGGAGUUGACUCGCGAUA
UCUUUACGGACCCGAUAUUUCUGCUAACAACGCUGCAGAAAUAUGGGCCGACUUUCUUAUCGAUUGAAAAUUCGAUAAGAAAG
CCCCAUUUGUUCGACUACCUUCAGGGCAUCGAGUUCCACACCCGGUUGAGGCCCGGGUACUUCGGUAAAGAUAGCUUCAACUA
CUGGUCGGGGAACUACGUGGAAACGAGGCCCUCGAUCGGUAGUUCAAAAACAAUAACAUCACCAUUCUACGGAGACAAAUCAA
CAGAACCAGUACAAAAACUAUCAUUCGACGGACAAAAAGUAUACAGAACAAUAGCAAACACAGACGUAGCAGCAUGGCCAAAC
GGAAAAGUAUACCUAGGAGUAACAAAAGUAGACUUCUCACAAUACGACGACCAAAAAAACGAAACAUCAACACAAACAUACGA
CUCAAAAAGAAACAACGGACACGUAUCAGCACAAGACUCAAUAGACCAACUACCACCAGAAACAACAGACGAACCACUAGAAA
AAGCAUACUCACACCAACUAAACUACGCAGAAUGCUUCCUAAUGCAAGACAGAAGAGGAACAAUACCAUUCUUCACAUGGACA
CACAGAUCAGUAGACUUCUUCAACACAAUAGACGCAGAAAAAAUAACACAACUACCAGUAGUAAAAGCAUACGCACUAUCAUC
AGGAGCAUCAAUAAUAGAAGGACCAGGAUUCACAGGAGGAAACCUACUAUUCCUAAAAGAAUCAUCAAACUCAAUAGCAAAAU
UCAAAGUAACACUAAACUCAGCAGCACUACUACAAAGAUACAGAGUAAGAAUAAGAUACGCAUCAACAACAAACCUAAGACUA
UUCGUACAAAACUCAAACAACGACUUCCUAGUAAUAUACAUAAACAAAACAAUGAACAAAGACGACGACCUAACAUACCAAAC
AUUCGACCUAGCAACAACAAACUCAAACAUGGGAUUCUCAGGAGACAAAAACGAACUAAUAAUAGGAGCAGAAUCAUUCGUAU
CAAACGAAAAAAUAUACAUAGACAAAAUAGAAUUCAUACCAGUACAACUAUAA

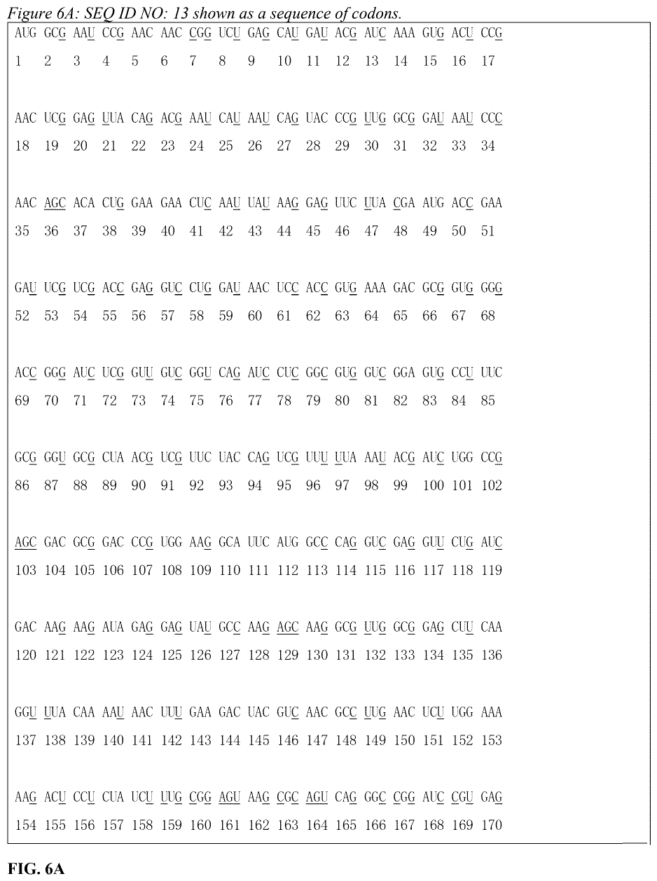

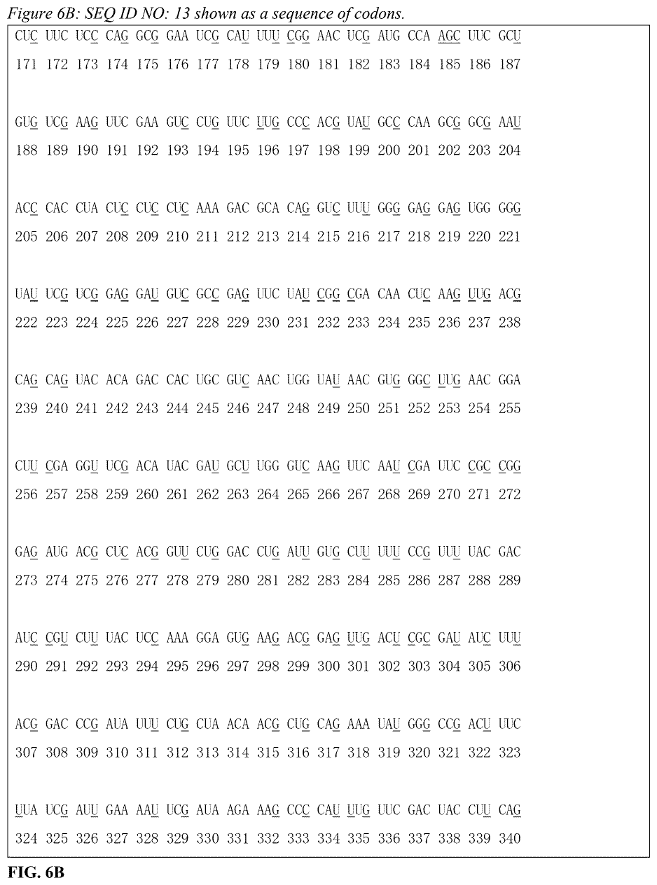

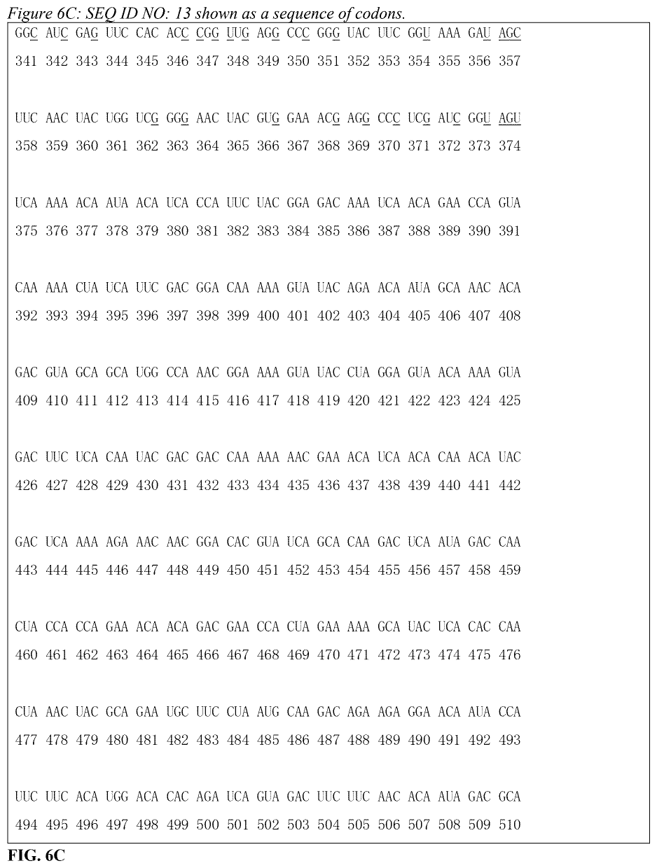

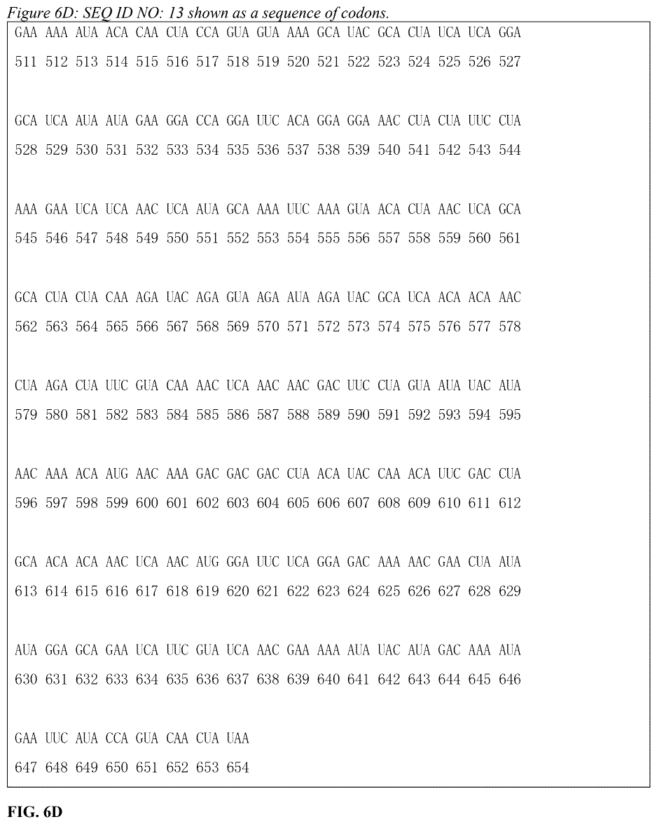

For convenience, FIG. 6 shows the ICS(PD₃₇₅) sequence SEQ ID NO: 13 with triplets numbered, with differences from reference underlined.

ICS(PD₂₈₂) is an example Cry3Bb1 protein CDS RNA at design level ~ 40%, with differences from reference underlined:

(example is using −MFE=633 from RNAFold as the estimator)

SEQ ID NO: 14 provides a design for a nucleic acid CDS sequence trained to a 40%
expression design level encoding of a Cry3Bb1 protein variant from US7705216

AUGGCGAAUCCGAACAACCGGUCUGAGCAUGAUACGAUCAAAGUGACUCCGAACUCGGAGUUACAGACGAAUCAUAAUCAGUA
CCCGUUGGCGGAUAAUCCCAACAGCACACUGGAAGAACUCAAUUAUAAGGAGUUCUUACGAAUGACCGAAGAUUCGUCGACCG
AGGUCCUGGAUAACUCCACCGUGAAAGACGCGGUGGGGACCGGGAUCUCGGUUGUCGGUCAGAUCCUCGGCGUGGUCGGAGUG
CCUUUCGCGGGUGCGCUAACGUCGUUCUACCAGUCGUUUUUAAAUACGAUCUGGCCGAGCGACGCGGACCCGUGGAAGGCAUU
CAUGGCCCAGGUCGAGGUUCUGAUCGACAAGAAGAUAGAGGAGUAUGCCAAGAGCAAGGCGUUGGCGGAGCUUCAAGGUUUAC
AAAAUAACUUUGAAGACUACGUCAACGCCUUGAACUCUUGGAAAAAGACUCCUCUAUCUUUGCGGAGUAAGCGCAGUCAGGGC
CGGAUCCGUGAGCUCUUCUCCCAGGCGGAAUCGCAUUUUCGGAACUCGAUGCCAAGCUUCGCUGUGUCGAAGUUCGAAGUCCU
GUUCUUGCCCACGUAUGCCCAAGCGGCGAAUACCCACCUACUCCUCCUCAAAGACGCACAGGUCUUUGGGGAGGAGUGGGGGU
AUUCGUCGGAGGAUGUCGCCGAGUUCUAUCGGCGACAACUCAAGUUGACGCAGCAGUACACAGACCACUGCGUCAACUGGUAU
AACGUGGGCUUGAACGGACUUCGAGGUUCGACAUACGAUGCUUGGGUCAAGUUCAAUCGAUUCCGCCGGGAGAUGACGCUCAC
GGUUCUGGACCUGAUAGUACUAUUCCCAUUCUACGACAUAAGACUAUACUCAAAAGGAGUAAAAACAGAACUAACAAGAGACA
UAUUCACAGACCCAAUAUUCCUACUAACAACACUACAAAAAUACGGACCAACAUUCCUAUCAAUAGAAAACUCAAUAAGAAAA
CCACACCUAUUCGACUACCUACAAGGAAUAGAAUUCCACACAAGACUAAGACCAGGAUACUUCGGAAAAGACUCAUUCAACUA
CUGGUCAGGAAACUACGUAGAAACAAGACCAUCAAUAGGAUCAUCAAAAACAAUAACAUCACCAUUCUACGGAGACAAAUCAA
CAGAACCAGUACAAAAACUAUCAUUCGACGGACAAAAAGUAUACAGAACAAUAGCAAACACAGACGUAGCAGCAUGGCCAAAC
GGAAAAGUAUACCUAGGAGUAACAAAAGUAGACUUCUCACAAUACGACGACCAAAAAAACGAAACAUCAACACAAACAUACGA
CUCAAAAAGAAACAACGGACACGUAUCAGCACAAGACUCAAUAGACCAACUACCACCAGAAACAACAGACGAACCACUAGAAA
AAGCAUACUCACACCAACUAAACUACGCAGAAUGCUUCCUAAUGCAAGACAGAAGAGGAACAAUACCAUUCUUCACAUGGACA
CACAGAUCAGUAGACUUCUUCAACACAAUAGACGCAGAAAAAAUAACACAACUACCAGUAGUAAAAGCAUACGCACUAUCAUC
AGGAGCAUCAAUAAUAGAAGGACCAGGAUUCACAGGAGGAAACCUACUAUUCCUAAAAGAAUCAUCAAACUCAAUAGCAAAAU
UCAAAGUAACACUAAACUCAGCAGCACUACUACAAAGAUACAGAGUAAGAAUAAGAUACGCAUCAACAACAAACCUAAGACUA
UUCGUACAAAACUCAAACAACGACUUCCUAGUAAUAUACAUAAACAAAACAAUGAACAAAGACGACGACCUAACAUACCAAAC
AUUCGACCUAGCAACAACAAACUCAAACAUGGGAUUCUCAGGAGACAAAAACGAACUAAUAAUAGGAGCAGAAUCAUUCGUAU
CAAACGAAAAAAUAUACAUAGACAAAAUAGAAUUCAUACCAGUACAACUAUAA

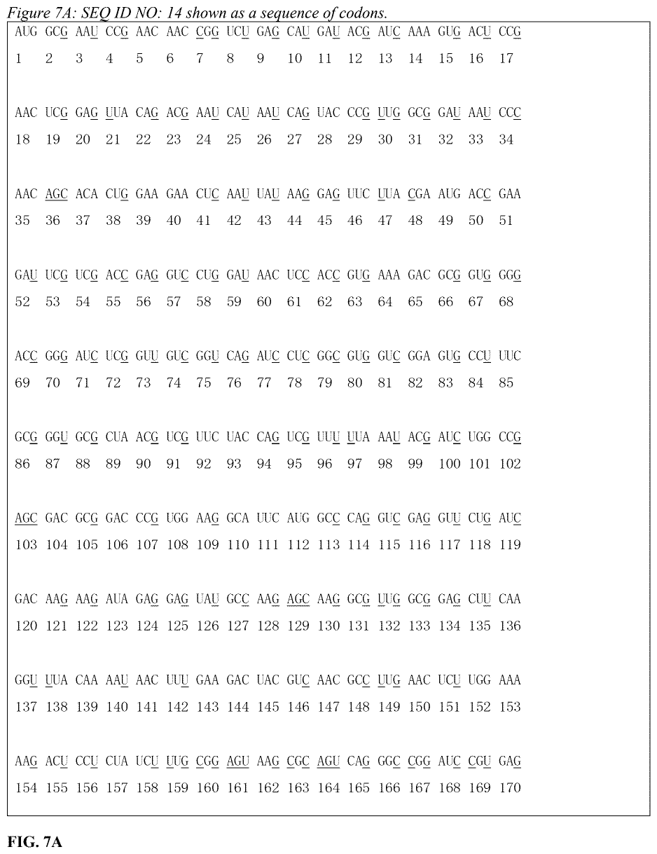

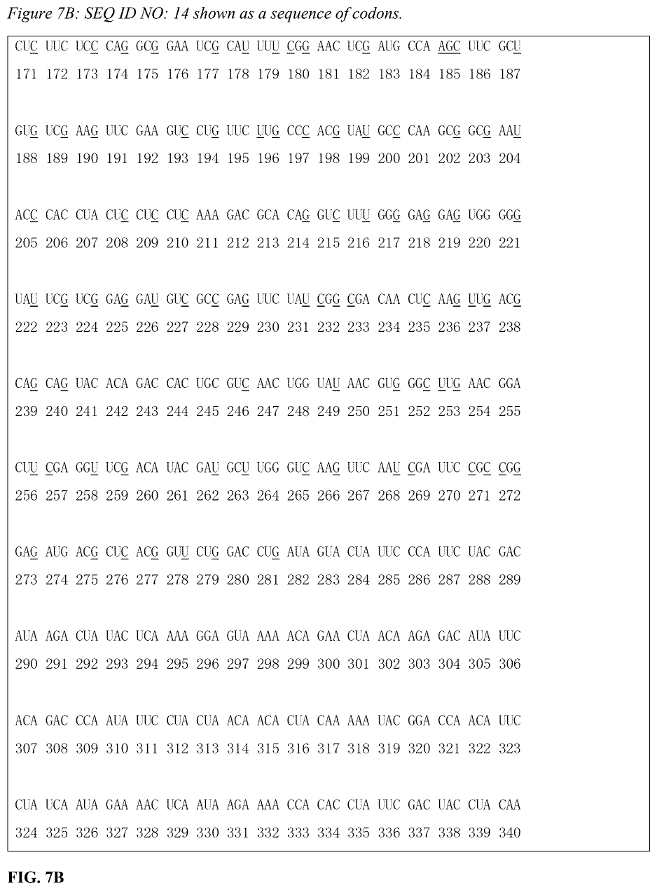

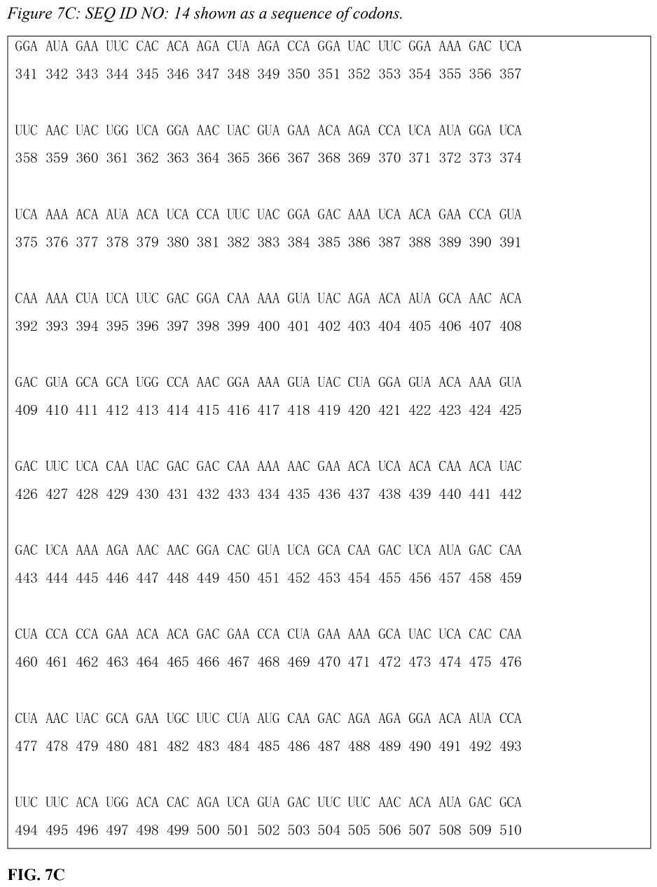

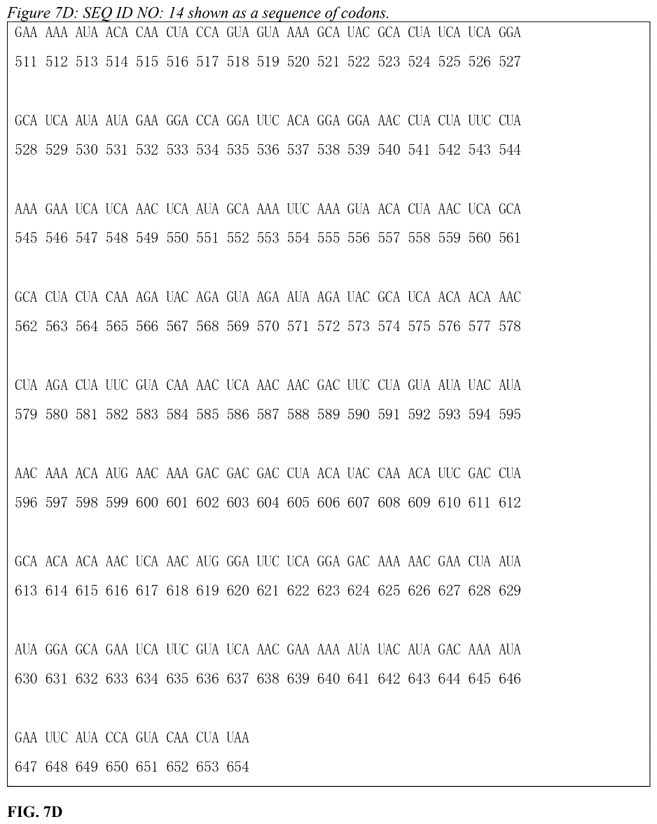

For convenience, FIG. 7 shows the ICS(PD₂₈₂) sequence SEQ ID NO: 14 with triplets numbered, with differences from reference underlined.

With the three expression levels, the assays may reveal that 40% is too low of a relative expression design level and that to ensure continued efficacy against growing insect resistance to Cry3Bb1, and may suggest the need to increase the design level to a higher level that increases the dose of the insect toxin. Depending on the results of the due experimentation with panel assays, one may choose to accept a design level or further refine the design level, optionally with a new panel of assays, and optionally repeating to accommodate a changing environment of insect resistance to Cry3Bb1.

### C. Example 3: Gene Training for Crop Research Involving Signal Analysis

A common task in crop research is to identify gene candidates that affect traits of interest. A number of different methods, including but not limited to, a solver such as via QTL analysis or GWAS, a simulation such as via a continuous process modeler, a systems biology tool such as via StochSS, or other mathematical computation, process, or algorithm, may be used to identify one or more genes of interest for further scrutiny.

In this example, we select genes in the vicinity of a putative QTL to help ascertain the nature of the cause of trait performance differences. In this particular example, a multi-gene example is used to illustrate our method, wherein the selection of the genes was done in part with results from a QTL solver analysis due to results indicating a region of genes that may affect tassel traits.

Although more or fewer proteins may be selected to further scrutinize the putative QTL, in this example the proteins selected for evaluation include the following:
Zm00001eb379110_P₀₀₁, Zm00001eb379120_P₀₀₁, Zm00001eb379130_P₀₀₁, Zm00001eb379140_P₀₀₁

For which the following respective or related annotations are found:

Transcription factor MYB39, Uncharacterized protein/Cytochrome P_{450 88}A1, Haloacid dehalogenase-like hydrolase domain-containing protein Sgpp, Haloacid dehalogenase-like hydrolase domain-containing protein Sgpp

For which the following respective relative expression design levels are found:
43%, 55%, 46%, 47%

While there are many approaches to design experiments that help tease apart a putative QTL, when variation in expression is suspected to be the cause (as opposed to other types of genetic variation), one approach is to increase and/or decrease the expression levels of the suspected causal genes. In doing so, one may carry out experimentation that tests the effect of changes in each gene's expression level individually or in pooled testing, where a genome may be tested that includes multiple genes with higher expression design level.

For the selection of expression levels, here we used an approach that amplifies existing expression levels for the target organism by a manually selected factor, but we note that additional approaches to identifying prospective expression levels of interest exist that may be determined through experimentation or in silico.

Coding Strand of Primary Transcript of Zm00001eb379110_T001 is @ 43%, with coding regions underlined:

SEQ ID NO: 15 provides the polynucleotide sequence of the coding strand of the DNA
region transcribed then spliced to the Zm00001eb379110_T001 CDS encoding of the
Zm00001eb379110_P001 variant of a Transcription factor MYB39 protein of Zea mays

ACGAACCCCACCTTCTTCGTTGCCTCTCTTCCTCCCTCTCCTCTCTTCCTTCCTCTCGATCGATCGTCATAGCAATCCAAGCT
AGCAAGCCCGTCCGTCGTCTCTCGTCCAAGAAGCATACAACTACTAATGGGGAGGTCACCGTGCTGCGACCAGGACGCGGGCG
TCAAGAAAGGCCCGTGGACGCCGGAGGAGGACAAGCTGCTGGTGGACTACATCAACGGCAACGGCCACGGGAGCTGGCGCCGC
CTGCCCAAGCACGCCGGCCTCAACCGCTGCGGCAAGAGCTGCCGCCTCCGCTGGACCAACTACCTCCGCCCGGACATCAAGCG
CGGCCGCTTCACCGACGACGAGGAGAAGCTCATCATCCACCTCCACTCCCTCCTCGGCAACAAGTGGTCCGCCATCGCCACCA
AGCTGCCCGGCCGGACCGACAACGAGATCAAGAACTACTGGAACACGCACCTCCGCAAGAAGCTGCTCGCCATGGGGATCGAC
CCCGTCACGCACCAGCGCCGCACCGACCTCAACCTCCTCGCGGCCGGCGGCTTCACCAACCTCCTCGCCGCCGCCAACCTCGC
GGCCGCCGCCGCTGGTGGCGGCCAAGCAGCTGGACCTGGAGGACCGCTCGCCGCCGCGCATGCTAGCTCCTGCTGGGACATCA
ACGCTCTCAGGCTCCAGGCCGACGCCGCCAAGTACCAGCTCCTCGAGGGCCTCGTCCGCGTGCTCACCGCCCCCGCCGCGCCC
ACCGTCGACCTCATGTCCCTCCTCGCCGCCGCCAATAATGGAGGCGCGCCCAGCGGCGGCGGCGGCCACCAGCAGCTGCTCGC
CGGAGTGGACCAAAGCACCGCCAGCAGGGTCGTGCAGCAGTACGACGGCATGCTCAGCCTTCCAGCGCTGACCAGCGCCGTGC
CGGCGGTGACGCAGGGCATGCCGTCCTCCTCCGCCTACTCCCTCTCTGGGCTCCTCAACGGCTTCTCCGGCGGTGCCGGGACT
ACGGCCGCTTTCGCCGGGGACGGGCTCAGCTCCACGGAGCTCGGCCACAGCGGGGCCACCACCGGGAGCAACGTGACTGCCGC
CGCGATGCCGCCACCTTTGGCAGCGGCGGCCAAGGGGTGCAGTGCAGGCAACGGCGGCGCCACGTCGTCGACCCCATCCCCGT
ACGAGGAGACGCCGGCGTCGAGCCCGTTCGACGGCCTGGACAGCCTCACGCTGGATTTTGACCCCAACAACGACAGTTGGAGA
GAATTGCTAGAGTGAGTATACCACCTATAGCATCGATCTAGCATACCATGCCATGCATGCATCAACTGTAGTGTTTGCTTATG
TATATTTTCTAGCTACAAAGCAGTATACATGCATGTAAATTATATACATACTAGCTAGACTAAGATTCAAAAAAAAAAAAAAA
ACTTTAGCGTACTAAATAGCATGTGTGTAGATACATATCCTGATTTAATTTGCTATTAGCTACAGGTATAGATGTATGTAAGT
TATATATATATATATATATATATATATATATATATATATATATATATATATATATATATATATATATATATATATATATATAT
ATATATATATATATAGACTAGATGCTATAAGATTCCAAAAAAATAAAACTTTAGCTTACTAAATATCATGTGTGTATATATAT
CCTTGTTTAATCTGCCAGCTAAAAGTATATATGCATGTAAATATGGATATACTACCTAGCTATGATTTCCAAAAAAAAGGCTT
AGCTTACGAAATAGTAACATGTATATAGTATTTATTTTCTTATTGTTGCCTCTGATCTCACCATGTATTTTCCTTCTCTTTGT
CCTTTGGTGCATGTGCAGGCAAATGTCATGGTTGAACAACCCCAATGATCAGCTGTGAG

Coding Strand of Primary Transcript of Zm00001eb379110_P₀₀₁ increased to 53% with coding regions underlined and differences from reference in bold with wavy underline:

provides a design for a polynucleotide sequence to help identify what needs
DNA editing with CDS trained to a 53% expression design level with putative
encoding of the Zm00001eb379110_P001 variant of a Transcription factor MYB39
protein of Zea mays
SEQ ID NO: 16
ACGAACCCCACCTTCTTCGTTGCCTCTCTTCCTCCCTCTCCTCTCTTCCTTCCTCTCGATCGATCGTCATAGCAATCCAAGCT

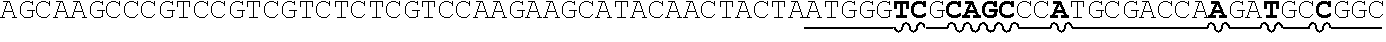

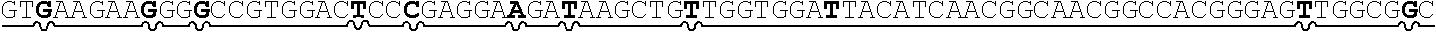

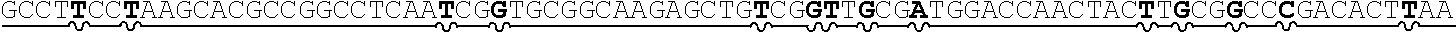

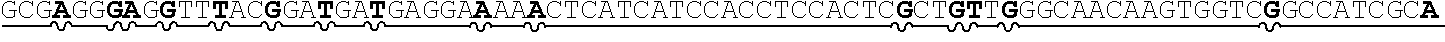

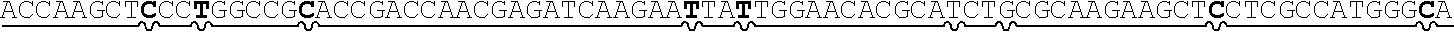

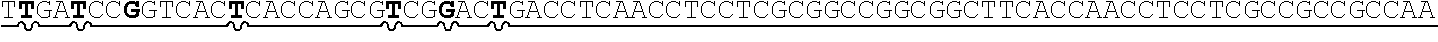

CCTCGCGGCCGCCGCCGCTGGTGGCGGCCAAGCAGCTGGACCTGGAGGACCGCTCGCCGCCGCGCATGCTAGCTCCTGCTGGG
ACATCAACGCTCTCAGGCTCCAGGCCGACGCCGCCAAGTACCAGCTCCTCGAGGGCCTCGTCCGCGTGCTCACCGCCCCCGCC
GCGCCCACCGTCGACCTCATGTCCCTCCTCGCCGCCGCCAATAATGGAGGCGCGCCCAGCGGCGGCGGCGGCCACCAGCAGCT
GCTCGCCGGAGTGGACCAAAGCACCGCCAGCAGGGTCGTGCAGCAGTACGACGGCATGCTCAGCCTTCCAGCGCTGACCAGCG
CCGTGCCGGCGGTGACGCAGGGCATGCCGTCCTCCTCCGCCTACTCCCTCTCTGGGCTCCTCAACGGCTTCTCCGGCGGTGCC
GGGACTACGGCCGCTTTCGCCGGGGACGGGCTCAGCTCCACGGAGCTCGGCCACAGCGGGGCCACCACCGGGAGCAACGTGAC
TGCCGCCGCGATGCCGCCACCTTTGGCAGCGGCGGCCAAGGGGTGCAGTGCAGGCAACGGCGGCGCCACGTCGTCGACCCCAT
CCCCGTACGAGGAGACGCCGGCGTCGAGCCCGTTCGACGGCCTGGACAGCCTCACGCTGGATTTTGACCCCAACAACGACAGT
TGGAGAGAATTGCTAGAGTGAGTATACCACCTATAGCATCGATCTAGCATACCATGCCATGCATGCATCAACTGTAGTGTTTG
CTTATGTATATTTTCTAGCTACAAAGCAGTATACATGCATGTAAATTATATACATACTAGCTAGACTAAGATTCAAAAAAAAA
AAAAAAACTTTAGCGTACTAAATAGCATGTGTGTAGATACATATCCTGATTTAATTTGCTATTAGCTACAGGTATAGATGTAT
GTAAGTTATATATATATATATATATATATATATATATATATATATATATATATATATATATATATATATATATATATATATAT
ATATATATATATATATATATAGACTAGATGCTATAAGATTCCAAAAAAATAAAACTTTAGCTTACTAAATATCATGTGTGTAT
ATATATCCTTGTTTAATCTGCCAGCTAAAAGTATATATGCATGTAAATATGGATATACTACCTAGCTATGATTTCCAAAAAAA
AGGCTTAGCTTACGAAATAGTAACATGTATATAGTATTTATTTTCTTATTGTTGCCTCTGATCTCACCATGTATTTTCCTTCT
CTTTGTCCTTTGGTGCATGTGCAGGCAAATGTCATGGTTGAACAACCCCAATGATCAGCTGTGAG

Coding Strand of Primary Transcript of Zm00001eb379120_T001 is @ 55%, with coding regions underlined:

SEQ ID NO: 17 provides the polynucleotide sequence of the coding strand of
the DNA region transcribed then spliced to the Zm00001eb379120_T001
transcript with CDS encoding of the Zm00001eb379120_P001 variant
of an uncharacterized protein of Zea mays

CCAAAAGCCGGCCACTTACTTGCGTCTGCATAATACTACAAGGCATCCGGCCTATATGCCGGGTGATCGATGATGATGATGAT
ATAAAACTCCGAGGACCAAAAAAGAAAAAACCGTACGTTACCAATTAAGAGCTTTTGCTTTCAAGAAGAATTTCTCTATACAG
GCGTGCATGGTGGGTGTTTGATTTCATTTTTGGAACGTAATGGTGTTTGATTTCTAGCACAGACAGAGACAGAGGGTGTGGCG
TTACACATCTCTGCCTATATATAAACAGGAGGCGTTGCGTGCAGAGCTGCCGGCTGGCGGTTGCCTCTCCTCCCCTCCCCTGC
CCTCGCAAACGCACGCACCACAGCATCGTCCTTCGTTCCATTCCGGCATTCCCATCCATATACCCCCTCGATCGGCCGGGATG
CTGGGTGTGGGGATGGCCGCGGCGGTGCTGCTCGGGGCCGTGGCGTTGCTGCTCGCGGACGCCGCTGCGAGGAGAGCGCACTG
GTGGTACAGGGAGGCGGCTGAGGCGGTGCTGGTCGGCGCCGTGGCTTTGGTGGTGGTGGACGCCGCGGCGCGGAGGGCGCACG
GGTGGTACAGGGAAGCGGCGCTGGGCGCGGCGCGGCGGGCGCGGCTGCCGCCGGGGGAGATGGGGTGGCCCTTGGTCGGCGGC
ATGTGGGCCTTCCTCCGCGCCTTCAAGTCCGGCAAGCCCGACGCCTTCATCGCCTCCTTCGTCCGGCGGTGAGTCAGTGCGCT
AATCCTCGATCTCCTCCCTCCTCCCCTGCACGCTCGCTCCGCTCTGCTCCTACCGAGGATGGGCCGGGCCCCACGTGTCATTG
TCCTCGTCGACACCTGCACCTGCACGCACCGGGCCCCACGGCCGTGTGAGCTTGTGGCGCATGGGTGCACCGGAGCAGCATTA
GCTAGCCAGCAGCAATGGATAGCTGTCGATTTTCTATCCCAGGCTGGTTAATTAATCCTCATCCCTTTGCCTGCTAAATAGGA
GTATTACTTGTACTGAAATCACCAAAATCTAGAACATAACCGTTATGACAGCTGTATATATAGAATATGAATAGTTAAATGGC
TCGTCAAGCCAAATTTTATCTAATCAACAGCAAAATAACTACATATATACAACAAAGTAACCATCTAAATAACTCATCAAGCT
ATTTGTCTCTTTAAATTGGCTCTCCCTCACAGGCCAAACTTGACTAGCTAAACTAGATAGATAGTCTATTGGATTGAGATGTT
ACACATAGAATGTAATCTTTATAGAAAAGTAAATAGATAGTCAAATAAAGAGAAAAAAAATGAAGAGTACTGTGAAGATGCTC
TACTAGTAGTACCAGAAATTAAGAAGTATATATAACTCACATAGGCATGCATTGCACTTGTAGTCGCAAGCAATTCACTGCTG
TTACTTTTGTAAACGAAGCAGTCAGACAGGAGAGCTATCGAGTATCAAAAGAAGGCCAGACCAGACTGAGACAGGGTAGAACG
ACGGTCGACGACGACGTCCCGCGCGCAGGAGAGGAAGGTAAAGGCAGTGGTGATTGGTGAATAATCACTGTCAGCCAGGCTAA
AAGCTCAGAGCATGTACAGTCATCAGAGTCAGTCAGACTGTGATGAGTGACATGATCCGACGTGAATGCCCCCACAAAAGGGC
ACATCCCATTCCGACTGACTGATTGCAACGCCACCACTCATCACTCGCCATCTTTGACAGTCTCCCTTTCCTGGATACATGCT
CGATGCTCTCCTAGTAAACACTAAACACCGTGCGTGATGCTCTGTTATTGATGCCCTGCTTGGCAGCTACCCCACCGCCATCC
CTGATTCTCCAGCCACATGCTGCCAAAAGCAACTAAAATTTGTCTCTGACGATTCTTCTGCCCTCGCGGCCGGTCCATCCAAA
GCGACTGGTTTTTTGTTTTTTTTTTTAATTCAGACACGAATATGTTCTGAACATCTATCTAGCTCTCTCTCTCTGAGCTAGAT
TTATGTGGTACGAAGAACCCTCCTGTTCAGATGGGCCTATGTATGATACGTGTTGCTCACTCGCTTGCTCCTCCTCGCAGTCT
CAATGGATGGATGAATCAGTTGTGCTGATCAAACAGACGAACAGTCAGATAGACAGGCAAGGCAACCAGACCATCTATCTGAC
CTGGACGCGCCATTAATTGCAGGTTCGGTCGCACGGGCGTGTACCGAAGCTTCATGTTCAGCAGCCCGACTGTGCTGGTGACG
ACGGCGGAGGGGTGCAAGCAGGTGCTGATGGACGACGACGCGTTCGTGACGGGGTGGCCCAAGGCGACGGTTGCGCTGGTCGG
GCCGCGGTCGTTCGTGGCGATGCCGTACGACGAGCACCGGCGCATCCGCAAGCTGACTGCCGCGCCCATCAACGGCTTCGACG
CGCTGACGGGGTACCTGCCCTTCATCGACCGCACCGTCACGTCGTCGCTGCGCGCGTGGGCGGACCACGGCGGCAGCGTCGAG
TTCCTGACGGAGCTGCGGCGCATGACGTTCAAGATCATCGTGCAGATCTTCCTGGGCGGCGCGGACCAGGCCACCACGCGCGC
GCTGGAGCGCAGCTACACGGAGCTCAACTACGGCATGCGCGCCATGGCCATCAACCTGCCCGGCTTCGCGTACCGCGGGGCGC
TGCGCGCGCGCCGCCGCCTCGTGGCCGTGCTGCAGGGCGTGCTGGACGAGCGCCGCGCCGCCAGGGCCAAGGGGGTCTCGGGC
GGAGGAGTGGACATGATGGACCGGCTGATCGAGGCCCAGGACGAGCGCGGGCGGCACCTGGACGACGACGAGATCATCGACGT
GCTCGTCATGTACCTCAACGCCGGCCACGAGTCGTCCGGCCACATCACCATGTGGGCCACCGTGTTCCTGCAGGAGAACCCGG
ACATGTTCGCGAGAGCAAAGGCAGGTTCTTTCTTTCTTTCTTTCTTTGTTGTTTAATTTTATCCTGAAAGCTAGCTGGACAAT
TAAGTGCGTGCTCTTCATTCCCATGCCACCAGGCGGAGCAGGAGGCGATCATGAGGAGCATCCCGTCGTCGCAGCGGGGGCTG
ACGCTCAGGGACTTCAGGAAGATGGAGTACCTGTCGCAGGTCTGCTCCGATCCATCCATTCTCTGACGATGCTAGCTTGCATC
AGTGCAAAAACATGCTCCATGGCCTGACTGACTGACTGATCCCGTGAACGAAGGTGATCGACGAGACGCTGCGGCTCGTCAAC
ATCTCCTTCGTCTCCTTCCGTCAGGCCACCAGAGACGTCTTCGTGAACGGTACGCTGCATGCTGTTGCTGTGCGGCTTCATTC
ATTCAGGAACCTCACCTCGCTCTGGAACAGGATACCTCATCCCCAAGGGGTGGAAGGTTCAGCTCTGGTACCGGAGCGTGCAC
ATGGACCCACAGGTGTACCCTGACCCCACCAAGTTCGACCCGTCGAGATGGGAGGGCCACTCGCCGAGAGCCGGCACGTTCCT
GGCGTTCGGGCTGGGCGCCAGGCTCTGCCCCGGCAACGACCTCGCCAAGCTGGAGATTTCCGTCTTCCTCCACCACTTCCTCC
TCGGCTACAAGTAACTAAATAACTATCTGTGTCGTCGAGACGACGATTATTCCTTTCTTTTTTTTTAGCTGGTCGAGCAGATG
AACTGTTGCTGATCGACGGGCATGCGTGCGTGCAGGCTGGCGAGGACGAACCCTAGGTGCCGGGTGAGGTACCTGCCGCACCC
GAGGCCGGTGGACAACTGCTTGGCCAAGATCACCAGAGTCGGTAGCTAGCTGCTAGATTGTGATGGAAGTACGAATGAAAAAA
AAGCCCAGCAGAAACTCTAGCGGCTGGGCCGGCTGGAAATTATGGGCCGGGCCATTGTGGTTGCCCCCCCTGTCTGTGCAAAA
ATAAAACAAGCAGAAAGGAATAAAAAAAAGGAAAAGTATTACTTCGAACGCTGAAATTCTTGGTCGGGTCGGGCCTGTTTTCG
GCCCATCTCAGGCAGCCAGGTGCGCTCGTGTCGTCCCACTGTCTCGAAATCAAAA

Coding Strand of Primary Transcript of Zm00001eb379120_P₀₀₁ increased to 65% with coding regions underlined and differences from reference in bold with wavy underline:

provides a design for a polynucleotide sequence to help identify what needs
DNA editing with CDS trained to a 65% expression design level encoding of a
Zm00001eb379120_P001 variant of an uncharacterized protein of Zea mays
SEQ ID NO: 18
CCAAAAGCCGGCCACTTACTTGCGTCTGCATAATACTACAAGGCATCCGGCCTATATGCCGGGTGATCGATGATGATGATGAT
ATAAAACTCCGAGGACCAAAAAAGAAAAAACCGTACGTTACCAATTAAGAGCTTTTGCTTTCAAGAAGAATTTCTCTATACAG
GCGTGCATGGTGGGTGTTTGATTTCATTTTTGGAACGTAATGGTGTTTGATTTCTAGCACAGACAGAGACAGAGGGTGTGGCG
TTACACATCTCTGCCTATATATAAACAGGAGGCGTTGCGTGCAGAGCTGCCGGCTGGCGGTTGCCTCTCCTCCCCTCCCCTGC
CCTCGCAAACGCACGCACCACAGCATCGTCCTTCGTTCCATTCCGGCATTCCCATCCATATACCCCCTCGATCGGCCGGGATG

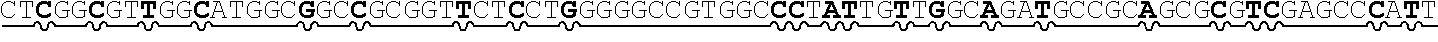

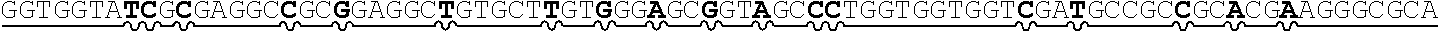

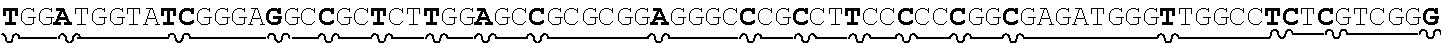

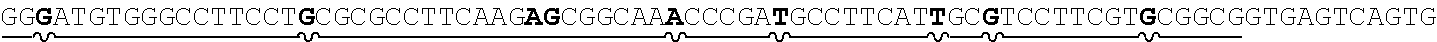

CGCTAATCCTCGATCTCCTCCCTCCTCCCCTGCACGCTCGCTCCGCTCTGCTCCTACCGAGGATGGGCCGGGCCCCACGTGTC
ATTGTCCTCGTCGACACCTGCACCTGCACGCACCGGGCCCCACGGCCGTGTGAGCTTGTGGCGCATGGGTGCACCGGAGCAGC
ATTAGCTAGCCAGCAGCAATGGATAGCTGTCGATTTTCTATCCCAGGCTGGTTAATTAATCCTCATCCCTTTGCCTGCTAAAT
AGGAGTATTACTTGTACTGAAATCACCAAAATCTAGAACATAACCGTTATGACAGCTGTATATATAGAATATGAATAGTTAAA
TGGCTCGTCAAGCCAAATTTTATCTAATCAACAGCAAAATAACTACATATATACAACAAAGTAACCATCTAAATAACTCATCA
AGCTATTTGTCTCTTTAAATTGGCTCTCCCTCACAGGCCAAACTTGACTAGCTAAACTAGATAGATAGTCTATTGGATTGAGA
TGTTACACATAGAATGTAATCTTTATAGAAAAGTAAATAGATAGTCAAATAAAGAGAAAAAAAATGAAGAGTACTGTGAAGAT
GCTCTACTAGTAGTACCAGAAATTAAGAAGTATATATAACTCACATAGGCATGCATTGCACTTGTAGTCGCAAGCAATTCACT
GCTGTTACTTTTGTAAACGAAGCAGTCAGACAGGAGAGCTATCGAGTATCAAAAGAAGGCCAGACCAGACTGAGACAGGGTAG
AACGACGGTCGACGACGACGTCCCGCGCGCAGGAGAGGAAGGTAAAGGCAGTGGTGATTGGTGAATAATCACTGTCAGCCAGG
CTAAAAGCTCAGAGCATGTACAGTCATCAGAGTCAGTCAGACTGTGATGAGTGACATGATCCGACGTGAATGCCCCCACAAAA
GGGCACATCCCATTCCGACTGACTGATTGCAACGCCACCACTCATCACTCGCCATCTTTGACAGTCTCCCTTTCCTGGATACA
TGCTCGATGCTCTCCTAGTAAACACTAAACACCGTGCGTGATGCTCTGTTATTGATGCCCTGCTTGGCAGCTACCCCACCGCC
ATCCCTGATTCTCCAGCCACATGCTGCCAAAAGCAACTAAAATTTGTCTCTGACGATTCTTCTGCCCTCGCGGCCGGTCCATC
CAAAGCGACTGGTTTTTTGTTTTTTTTTTTAATTCAGACACGAATATGTTCTGAACATCTATCTAGCTCTCTCTCTCTGAGCT
AGATTTATGTGGTACGAAGAACCCTCCTGTTCAGATGGGCCTATGTATGATACGTGTTGCTCACTCGCTTGCTCCTCCTCGCA
GTCTCAATGGATGGATGAATCAGTTGTGCTGATCAAACAGACGAACAGTCAGATAGACAGGCAAGGCAACCAGACCATCTATC

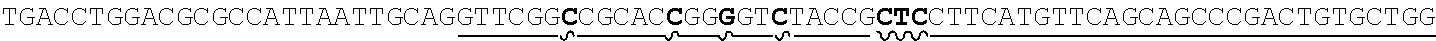

TGACGACGGCGGAGGGGTGCAAGCAGGTGCTGATGGACGACGACGCGTTCGTGACGGGGTGGCCCAAGGCGACGGTTGCGCTG
GTCGGGCCGCGGTCGTTCGTGGCGATGCCGTACGACGAGCACCGGCGCATCCGCAAGCTGACTGCCGCGCCCATCAACGGCTT
CGACGCGCTGACGGGGTACCTGCCCTTCATCGACCGCACCGTCACGTCGTCGCTGCGCGCGTGGGCGGACCACGGCGGCAGCG
TCGAGTTCCTGACGGAGCTGCGGCGCATGACGTTCAAGATCATCGTGCAGATCTTCCTGGGCGGCGCGGACCAGGCCACCACG
CGCGCGCTGGAGCGCAGCTACACGGAGCTCAACTACGGCATGCGCGCCATGGCCATCAACCTGCCCGGCTTCGCGTACCGCGG
GGCGCTGCGCGCGCGCCGCCGCCTCGTGGCCGTGCTGCAGGGCGTGCTGGACGAGCGCCGCGCCGCCAGGGCCAAGGGGGTCT
CGGGCGGAGGAGTGGACATGATGGACCGGCTGATCGAGGCCCAGGACGAGCGCGGGCGGCACCTGGACGACGACGAGATCATC
GACGTGCTCGTCATGTACCTCAACGCCGGCCACGAGTCGTCCGGCCACATCACCATGTGGGCCACCGTGTTCCTGCAGGAGAA
CCCGGACATGTTCGCGAGAGCAAAGGCAGGTTCTTTCTTTCTTTCTTTCTTTGTTGTTTAATTTTATCCTGAAAGCTAGCTGG
ACAATTAAGTGCGTGCTCTTCATTCCCATGCCACCAGGCGGAGCAGGAGGCGATCATGAGGAGCATCCCGTCGTCGCAGCGGG
GGCTGACGCTCAGGGACTTCAGGAAGATGGAGTACCTGTCGCAGGTCTGCTCCGATCCATCCATTCTCTGACGATGCTAGCTT
GCATCAGTGCAAAAACATGCTCCATGGCCTGACTGACTGACTGATCCCGTGAACGAAGGTGATCGACGAGACGCTGCGGCTCG
TCAACATCTCCTTCGTCTCCTTCCGTCAGGCCACCAGAGACGTCTTCGTGAACGGTACGCTGCATGCTGTTGCTGTGCGGCTT
CATTCATTCAGGAACCTCACCTCGCTCTGGAACAGGATACCTCATCCCCAAGGGGTGGAAGGTTCAGCTCTGGTACCGGAGCG
TGCACATGGACCCACAGGTGTACCCTGACCCCACCAAGTTCGACCCGTCGAGATGGGAGGGCCACTCGCCGAGAGCCGGCACG
TTCCTGGCGTTCGGGCTGGGCGCCAGGCTCTGCCCCGGCAACGACCTCGCCAAGCTGGAGATTTCCGTCTTCCTCCACCACTT
CCTCCTCGGCTACAAGTAACTAAATAACTATCTGTGTCGTCGAGACGACGATTATTCCTTTCTTTTTTTTTAGCTGGTCGAGC
AGATGAACTGTTGCTGATCGACGGGCATGCGTGCGTGCAGGCTGGCGAGGACGAACCCTAGGTGCCGGGTGAGGTACCTGCCG
CACCCGAGGCCGGTGGACAACTGCTTGGCCAAGATCACCAGAGTCGGTAGCTAGCTGCTAGATTGTGATGGAAGTACGAATGA
AAAAAAAGCCCAGCAGAAACTCTAGCGGCTGGGCCGGCTGGAAATTATGGGCCGGGCCATTGTGGTTGCCCCCCCTGTCTGTG
CAAAAATAAAACAAGCAGAAAGGAATAAAAAAAAGGAAAAGTATTACTTCGAACGCTGAAATTCTTGGTCGGGTCGGGCCTGT
TTTCGGCCCATCTCAGGCAGCCAGGTGCGCTCGTGTCGTCCCACTGTCTCGAAATCAAAA

Coding Strand of Primary Transcript of Zm00001eb379130_P_{001 @ 46}%, with coding regions underlined:

SEQ ID NO: 19 provides the polynucleotide sequence of the coding strand of the
DNA region transcribed then spliced to the Zm00001eb379130_T001 transcript with
CDS encoding of the Zm00001eb379130_P001 variant of an Haloacid dehalogenase-
like hydrolase domain-containing protein Sgpp of Zea mays

CCACAAAAAAAAACACACACACAGGGGAGGGAGACGAGACGAGATCCAAAATTCCCTGGCGGTGGGCTGTGGGCTGTGGCTGG
TAATAAAGCTCGCGCCACGGGAGCCACGCACGCGGACGCCGGCATATTCGCCAAACGCATCGCATCGGCCTCAGCCACCGGCG
CGGCTGCCCTCCTCTTCTCCGTCGATCCGGAGAGGACTTTCTCTCGCCTGCTCGTCTCTCCCGCCCCCGCCGGCCGGCCGGTC
CTCTTCCCGGCCCACTCCATTCCGATCGACCATGGCGGCTACCGCCCCCAACGGCAATCCCACTGTCAGGTACGGCTCCCGCC
TCGTTCCATCATGCGATGAGAGTTAACTACCCCCCCCCCCCCCCCCCCCCCCCCCCCCGTCGCGTGAACAGAGCGCCATCCAA
TGACGCCACTTCGTTGCGGTTTTAGATTTACCACCCACTCGTTCCGAGCCTGTGCGTGGTCAATATGTGTGTGAGATGAATTA
CATACTGTTTCTGCTCTTCTTCTTCTGAAACCGTCGAGTTGCGTGACTACCATCGAGCGATGCACAGCCTGAGATTTATCGCG
CGCGCACAAACACGCATACTCGTTCCAAGTTCCTGATCACCTGATGATTCAAATGATGGGCAATTGCCAATTGGGCATGATCT
GCAGCTCCCTCGCGGCAACCGTTCCGGTCCAGGCGGTTCTGTTCGACATCGACGGGACCCTGTGCGACTCGGATCCCCTCCAC
CACGTCGCTTTCCAAGAGATGCTTCTCGAGGTACGGTGGAACGTCGCTTTTCCCTGTTTCACTTTTCTTTTTTTAAACCAACG
AGCTGCCTGCGATGTGATGTGGTTTGATTTACGAAACCAAAATGGCTGGCATGGCATACGCAGATCGGGTACAACAATGGCGT
GCCGATAGACGAGGAGTTCTTCATCAAGAACATCGCTGGGAGGAGTGACGTCGAAGCTGCCCAGAACCTGTTCCCGGACTGGG
AGCTTGAGAAGGGGCTCAAATTCCTAGAGGACAAGGAAGCCAAATACAGGAGGTACGTACAAATGATAGCTGTGGTTTTCCTC
AGTATTAGATCAACTGAAAGATTCAGAGAGTAAGCGTGTTGTCCATTGTTACAAGTTTACAACAGTAAACCCCTTTGCGTACC
GTAGCACATACATAAATAACATCCATTTCAGCGCTATCACGCAAAAAAAAAACACCAACTTTTTGCTACTCAGAGAGATCGAT
TACTAGGTTGTGGAGAGTTCTGTCATTTCCTACAGCGCCCATTCGCTGCTGATTCTGCGGAATGTGATCCTTTCATAATCATT
ACAATACATTGCATTGCATTACAGAACTGATAGGTTCGTTATGACAACGTGCATGAGTGACTGAATGTTTCTAGTTGTTGGTT
CTTTTTATTCAGTCTGGCAAAAGAGCGCTTGGTGCCTGTGAAAGGCCTCGCGAAGGTGGTCCAGTGGGTGAAAGACCACGGGT
ACAAGCGCGCGGCGGTGACCAACGCCCCCAGGATCAACGCGGAGCTGATGATCTCGCTCCTGGGCCTCTCGGACTTCTTCCAG
GCCGTCATCGTCGGAGGCGAGTGCGAGCAGCCAAAGCCCGCGCCGTACCCTTACCTCAGGGCCCTCAAGGAGCTCCAGGTGTC
CGCAGAGCACAGCTTCGTCTTCGAGGTAAAATCAAACGACCTTGTGACTGGGATTCTGTGATCTGATGGTTGTATTACAGGAG
TAGTCACTGATAAAAGATGAGATTTCTTTTTGGGTTGGTGTAGGATTCTCCTGCTGGTATCCGGGCTGGCGTCGCGGCGGGGA
TGCCTGTCGTCGGTGTGGCGACGAGGAACCCGGAGAAGTCCTTGGTGGAAGCAGGAGCCGCGTTGCTCGTCAAGGACTACGAA
GATCCTAAGCTCTGGGCAGCGCTTGAGGAGATGGACGGAGAGGAAGCTAAGCTGAAGAAGGCCAGCGAATGATCTGATATGAC
GATGAGCCATTGATTAGATTTATTGCAGATTAAATCTGCCTCCAGCATGATTCTGTTCCTAGTTATGCGCTGCGGAGAGAGCT
CACCTCATCAGGGTATTTATGAGATTCTAGCTTCGTATATTGTTTTTCTTCTTCTTGATTGAACTGAGATCAAATAAGACAAA
GCTGCTGTGCCTTGTTGTGTGCCATCTGTGTATGAACTTTTCGGTTCAGCCTCCAGCAGTCTTTAATCTGAAAGAAAGACGAG
GTAGGAATTCTCTCCCTCAGGGAAGATGTGTGAAGAATGCACCGTGATGCTTGTTGCTTGTCGAATCCATGGACGTCATCCTT
TTTGAGCTTCAGCTCTGCTACGCAAACCGTGTTGAAAGTGGTTTCGGGGGCTATGCAGCA

Coding Strand of Primary Transcript of Zm00001eb379130_P₀₀₁ increased to 56% with coding regions underlined and difference from reference in bold with wavy underline:

provides a design for a polynucleotide sequence to help identify what needs
DNA editing with CDS trained to a 56% expression design level encoding of a
Zm00001eb379130_P001 variant of an Haloacid dehalogenase-like hydrolase domain-
containing protein Sgpp of Zea mays
SEQ ID NO: 20
CCACAAAAAAAAACACACACACAGGGGAGGGAGACGAGACGAGATCCAAAATTCCCTGGCGGTGGGCTGTGGGCTGTGGCTGG
TAATAAAGCTCGCGCCACGGGAGCCACGCACGCGGACGCCGGCATATTCGCCAAACGCATCGCATCGGCCTCAGCCACCGGCG
CGGCTGCCCTCCTCTTCTCCGTCGATCCGGAGAGGACTTTCTCTCGCCTGCTCGTCTCTCCCGCCCCCGCCGGCCGGCCGGTC

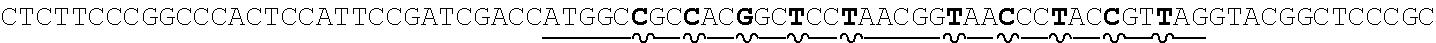

CTCGTTCCATCATGCGATGAGAGTTAACTACCCCCCCCCCCCCCCCCCCCCCCCCCCCCGTCGCGTGAACAGAGCGCCATCCA
ATGACGCCACTTCGTTGCGGTTTTAGATTTACCACCCACTCGTTCCGAGCCTGTGCGTGGTCAATATGTGTGTGAGATGAATT
ACATACTGTTTCTGCTCTTCTTCTTCTGAAACCGTCGAGTTGCGTGACTACCATCGAGCGATGCACAGCCTGAGATTTATCGC
GCGCGCACAAACACGCATACTCGTTCCAAGTTCCTGATCACCTGATGATTCAAATGATGGGCAATTGCCAATTGGGCATGATC

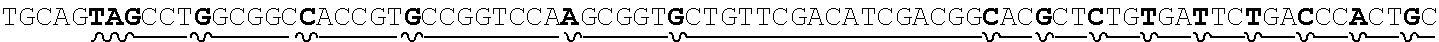

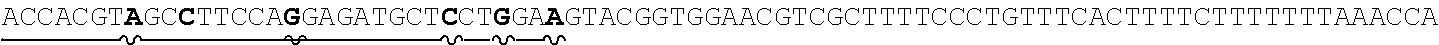

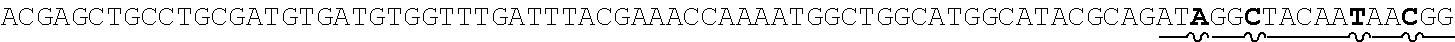

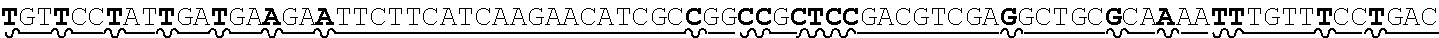

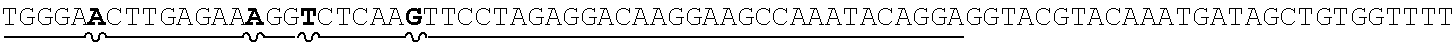

CCTCAGTATTAGATCAACTGAAAGATTCAGAGAGTAAGCGTGTTGTCCATTGTTACAAGTTTACAACAGTAAACCCCTTTGCG
TACCGTAGCACATACATAAATAACATCCATTTCAGCGCTATCACGCAAAAAAAAAACACCAACTTTTTGCTACTCAGAGAGAT
CGATTACTAGGTTGTGGAGAGTTCTGTCATTTCCTACAGCGCCCATTCGCTGCTGATTCTGCGGAATGTGATCCTTTCATAAT
CATTACAATACATTGCATTGCATTACAGAACTGATAGGTTCGTTATGACAACGTGCATGAGTGACTGAATGTTTCTAGTTGTT
GGTTCTTTTTATTCAGTCTGGCAAAAGAGCGCTTGGTGCCTGTGAAAGGCCTCGCGAAGGTGGTCCAGTGGGTGAAAGACCAC
GGGTACAAGCGCGCGGCGGTGACCAACGCCCCCAGGATCAACGCGGAGCTGATGATCTCGCTCCTGGGCCTCTCGGACTTCTT
CCAGGCCGTCATCGTCGGAGGCGAGTGCGAGCAGCCAAAGCCCGCGCCGTACCCTTACCTCAGGGCCCTCAAGGAGCTCCAGG
TGTCCGCAGAGCACAGCTTCGTCTTCGAGGTAAAATCAAACGACCTTGTGACTGGGATTCTGTGATCTGATGGTTGTATTACA
GGAGTAGTCACTGATAAAAGATGAGATTTCTTTTTGGGTTGGTGTAGGATTCTCCTGCTGGTATCCGGGCTGGCGTCGCGGCG
GGGATGCCTGTCGTCGGTGTGGCGACGAGGAACCCGGAGAAGTCCTTGGTGGAAGCAGGAGCCGCGTTGCTCGTCAAGGACTA
CGAAGATCCTAAGCTCTGGGCAGCGCTTGAGGAGATGGACGGAGAGGAAGCTAAGCTGAAGAAGGCCAGCGAATGATCTGATA
TGACGATGAGCCATTGATTAGATTTATTGCAGATTAAATCTGCCTCCAGCATGATTCTGTTCCTAGTTATGCGCTGCGGAGAG
AGCTCACCTCATCAGGGTATTTATGAGATTCTAGCTTCGTATATTGTTTTTCTTCTTCTTGATTGAACTGAGATCAAATAAGA
CAAAGCTGCTGTGCCTTGTTGTGTGCCATCTGTGTATGAACTTTTCGGTTCAGCCTCCAGCAGTCTTTAATCTGAAAGAAAGA
CGAGGTAGGAATTCTCTCCCTCAGGGAAGATGTGTGAAGAATGCACCGTGATGCTTGTTGCTTGTCGAATCCATGGACGTCAT
CCTTTTTGAGCTTCAGCTCTGCTACGCAAACCGTGTTGAAAGTGGTTTCGGGGGCTATGCAGCA

Coding Strand of Primary Transcript of Zm00001eb379140_T001 @ 47%, with coding regions underlined:

SEQ ID NO: 21 provides the polynucleotide sequence of the coding strand of the
DNA region transcribed then spliced to the Zm00001eb379140_T001 transcript with
CDS encoding of the Zm00001eb379140_P001 variant of a different Haloacid
dehalogenase-like hydrolase domain-containing protein Sgpp of Zea mays

TGGAACATGAGAATGTGAGAGACTACCACCAACCGCGCCAACTGAACCACGGAATCCTCACCAACTCTCCCTGCCTCTTCAGT
CTTCTCCTTATAAAATGTGCAGGCAGCATCAGCACTCGAGCAGTGACCACAAGACCAAAAGAAGCCTCAGTTTTGGCATCCAG
CAGCAACTGGCCATGGAAAGGTAATCATCCAGAGTATTTTCTGCATGATTTCACTCAGTTCTATTCCCCCATTCCCAGAGAGA
CAGAAACTGTCAAGATTTCGTTTCAATCCGACCAAAACTGTCAAGATTTCATTTCAATCCGAGCAAGTTCTCCGCAACCATGG
CCGTCCCAGTTTAAAGTTTTATCTTCTTTCCCTGTGTGTGTGTGTGTGGTTGCTTTGCAGCAGTGGGATCGGCAGAGTGGCCC
CACTGGAGGCCGTCCTCTTCGACATCGACGGCACCATGGCCATCTCCGACCCGTTCCACCACCGGGCCACCTCGGAGATGCTC
CTCAAGGTGGGCTACAACAACGGCGTGCCCATCACGCCCGAGTTCGGCATGGCGAACATGGCCGGCCGGAGCAACGAGCAGAT
CGGCCGCTTCCTGTTCCCGGACTGGGACCAGCGCCGCCTCGACGCCTTCTTCGCCGAGAAGGAGGCGCTCTTCGCCAGGTACG
CCGCCGAGGGGCTCCGGGAGATCGCCGGCCTCACGCCGCTCTGCCGCTGGGCCGCCGGCCGCGGGCTCAGGCGCGCCGCCGTC
ACCAACGCACCCAGGGCCAACGCCGAGCTCATGATCTCCATCCTCGGCCTCTCCGACTTTTTTTCCCTCGTCGTCACCGCCGA
GGAGTGCGGCCGATCCAAGCCCTACCCTGACCCGTACCTCAGGGCGCTCGACCTGCTCGGCGTCTCGCCGGACCACGCGCTCG
TGTTCGAGGACTCCACCACTGGCGTGCAGGCCGGCATTGCGGCAGGGATGCCGGTGGTTGCCATCGCCGAGGAAAGCAGAGAG
GACAAGCTTCTCGCCGTCGGCGCAACGCTCGTCATCAGGGACTACGAAGACCCCAAGCTATGGGCGGCTTTGGACAAACTGGA
CACCACCAGGCCTCAAGCTGCTGCTGCTGAGACCAATGGAAAACTCACACAACTGTAGCTGAGGAACACAAGTGCTAGTAGCC
AAAAACAATTTCTTGCAATATTCCACTTCCACGCATGATTCAATAACAACTCATTCGTGTATGTATGATGTATCTTGATGAGA
TGCATACAAAATTGCCTGTAGGCCAGTCTGTTACAGACAAATGAAAATGGAAACAAAACTGATCGCGATAACATGCAGATTTC
TATTTGTATCTAAACTACTCTACCACAGGCTCTCACATGAGATTATAATGTGCTTCAGAAATGGGTAAAAGCTGGTGACGGTG
CACACTCCTTGCAAACATGGTACAGACACCACATTTTTTTCACAAGGTTGTCCTCATATACACACTAACATGTGGACAGCTGG
T

Coding Strand of Primary Transcript of Zm00001eb379140_P₀₀₁ increased to 57% with coding regions underlined and differences from reference bold with wavy underline:

provides a design for a polynucleotide sequence to help identify what needs
DNA editing with CDS trained to a 57% expression design level encoding of a
Zm00001eb379140_P001 variant of the preceding Haloacid dehalogenase-like hydrolase
domain-containing protein Sgpp of Zea mays
SEQ ID NO: 22
TGGAACATGAGAATGTGAGAGACTACCACCAACCGCGCCAACTGAACCACGGAATCCTCACCAACTCTCCCTGCCTCTTCAGT

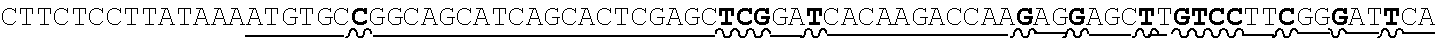

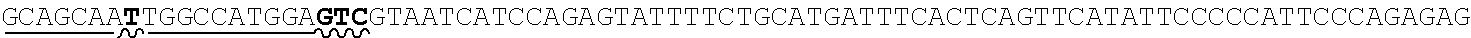

ACAGAAACTGTCAAGATTTCGTTTCAATCCGACCAAAACTGTCAAGATTTCATTTCAATCCGAGCAAGTTCTCCGCAACCATG

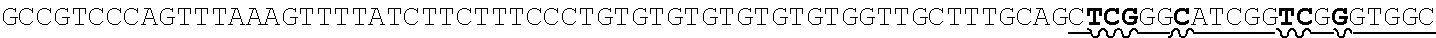

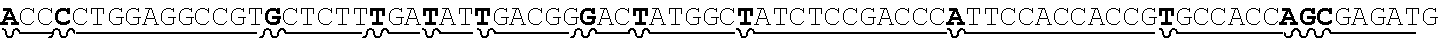

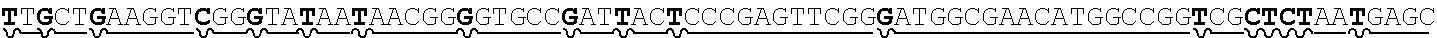

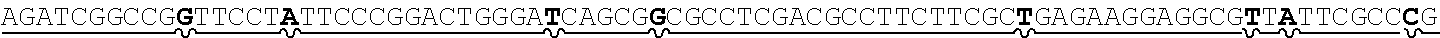

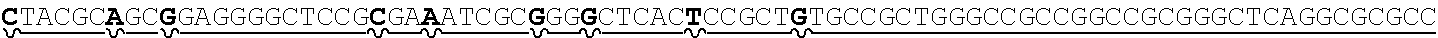

GCCGTCACCAACGCACCCAGGGCCAACGCCGAGCTCATGATCTCCATCCTCGGCCTCTCCGACTTTTTTTCCCTCGTCGTCAC
CGCCGAGGAGTGCGGCCGATCCAAGCCCTACCCTGACCCGTACCTCAGGGCGCTCGACCTGCTCGGCGTCTCGCCGGACCACG
CGCTCGTGTTCGAGGACTCCACCACTGGCGTGCAGGCCGGCATTGCGGCAGGGATGCCGGTGGTTGCCATCGCCGAGGAAAGC
AGAGAGGACAAGCTTCTCGCCGTCGGCGCAACGCTCGTCATCAGGGACTACGAAGACCCCAAGCTATGGGCGGCTTTGGACAA
ACTGGACACCACCAGGCCTCAAGCTGCTGCTGCTGAGACCAATGGAAAACTCACACAACTGTAGCTGAGGAACACAAGTGCTA
GTAGCCAAAAACAATTTCTTGCAATATTCCACTTCCACGCATGATTCAATAACAACTCATTCGTGTATGTATGATGTATCTTG
ATGAGATGCATACAAAATTGCCTGTAGGCCAGTCTGTTACAGACAAATGAAAATGGAAACAAAACTGATCGCGATAACATGCA
GATTTCTATTTGTATCTAAACTACTCTACCACAGGCTCTCACATGAGATTATAATGTGCTTCAGAAATGGGTAAAAGCTGGTG
ACGGTGCACACTCCTTGCAAACATGGTACAGACACCACATTTTTTTCACAAGGTTGTCCTCATATACACACTAACATGTGGAC
AGCTGGT

### D. Example 4: Gene Training for Crop Research Involving Exploratory Analysis

In this example, an example user interface is demonstrated to explore putative networks formed by enzymes and metabolites, allowing one to create hypotheses, conduct controlled expression perturbation analyses, and enable user-directed automated expression design prescribing gene edits.

This example illustrates one of the approaches to a user interface wherein one may set or change the expression design of genes with a user input device, with the specified designs implemented into the sequence representing the original DNA. This example is an exploration of the effect of setting or changing expression of two enzymes putatively involved in photorespiration.

The two genes selected can be found in the Kegg Photorespiration module zma_M00532 via Kegg reactions R01334 and R01388, via Kegg orthologs K19269 and K15919 respectively, via Kegg gene entries 100191272 and 100191765, respectively, with Zea Mays gene sequences equal to transcripts Zm00001eb064870_T001 and Zm00001eb299920_T001, respectively, with relative expression design levels of 23.1% and 59.6%, respectively, and annotated as B4F880_MAIZE Phosphoglycolate phosphatase and B4FA28_MAIZE Glyoxylate reductase, respectively, in uniprotkb last updated 2008 Sep. 23.

In particular, the selection of these two enzymes for exploration is in part due to their naturally low and high expression design levels, respectively, and towards an exploratory analysis of the result of expression perturbation that is a higher resolution approach to perturbation analysis, allowing for a greater precision in dynamic range and control of expression than existing perturbation methods such as gene knockouts, gene insertions, promoter augmentations, and protein sequence perturbations. Together with existing perturbation methods, precise expression perturbation may be used to reveal insights towards improving agricultural crops and livestock or carbon capture.

In this case, the user selects the circle representing Kegg compound C00160 (Glycolate) and associated reactions, which brings up the transcript identifiers associated with the Kegg genes associated with Kegg orthologs associated with the selected Kegg reactions. Then the user input device (a computer mouse or touchpad in this example) is used to set the expression design level by clicking the bar pertaining to the transcript of interest (on the right side of the screen)—the greater the filled area, the greater the expression design level.

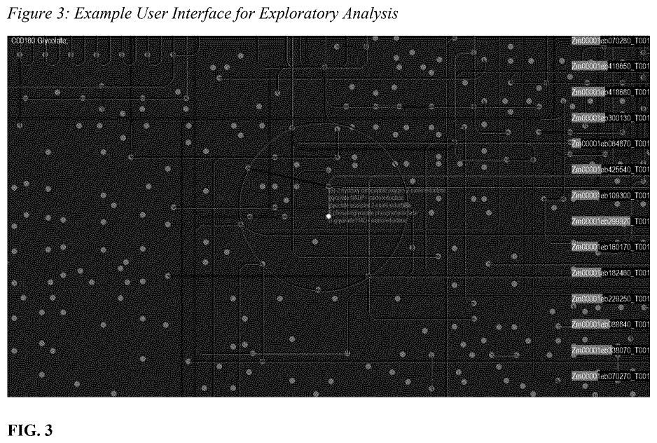

In this example, the user clicked to design a sequence that putatively decreases the expression design level of protein Zm00001eb064870_P₀₀₁ to 11% and clicked to design a sequence that putatively increases expression design level of protein Zm00001eb299920_P₀₀₁ to 74% to explore the impact of amplifying the natural expression design levels. When done setting expression design levels, the software automation outputs the sequences representing polynucleotides for use in gene editing, modification, or replacement in the DNA, wherein the expression design levels of Zm00001eb064870_P₀₀₁ and Zm00001eb299920_P₀₀₁ are updated to 11% and 74%, respectively, as illustrated in FIG. 3.

Original expression of Zm00001eb064870_T001 is @ 23% with coding regions underlined:

SEQ ID NO: 23 provides the polynucleotide sequence of the coding strand of the
DNA region transcribed then spliced to the Zm00001eb064870_T001 transcript with
CDS encoding of the Zm00001eb064870_P001 variant of a Phosphoglycolate
phosphatase protein of Zea mays

AGTTCTTTACCACTACCGGCCCAGACACCTAAACGATGACACAGCGTGCACCCACCACCACCAGCGCCGAGCGCCGGCCGCGA
TACCGTCGTGTCCCCCTCCCCACCGCATTAGCGCCGGCCGACAAAATTACTAGGTGCTTAATTTAACTACTAGTGGCCCTAGG
CGTGAAACTTCACCAGCTCCGCCACTGCCCATGGCCAACGGCCGTCCGGACCCTCGCTGCGTCGTCCTCACCGCCGACACTGC
CCGATCCCTCGTAGATTCCGTCGACGCCTTCCTCTTCGACTGCGATGGTATCTGATTGTTCACCTTTCTCCATTTTGTTTGTC
TGTTTTTTTTTCTTCGCTTCAGATTTTGACCCCTGCTTCGCTCTGACCGGTAATTCTAATTGCTTCTCTGAATTTGTCTAGGG
GTCATTTGGAAGGGAGATAAGCTCATTGAAGGGGTCCCAGAGACGATGGAGCTACTGAGGAAACTGGTATGGGAACATCCCTT
TATTACTATTTAAGTATTGAATTCGTTTCCTCATTTTCAATAGTTCAACTGTCAGGCAAATAAATGCCAAATGATCAAAACAA
GTACATAATTAAGGGCTAGCTTGTTTTGTCGTGCCTTAAAAAAAAAAACTGGTTCTAGATTGATTGGGGCTTTGAAGAATCTG
GTTCTCCAAAGTTGCATCTATTTAAGCTAAAATAGAGACAGGCAGTAAGTGCAACCACACCACACCAGGCACCAGATACCGTA
ATGAAATGAAAATCATTGACACTACTACTACTACATTGCTTTAAAGTTATTCTGGTTTTGTTCATCTACATAACTCCTGTCTG
AAACAAGGATAATAGGCCTATATCTTTTTTCTATGTGAATTCTAGCATGATGAACTCACCTCATTAACCAAGATGTGATCTTT
GTTGCGCCATTATTTAAGACATTGCTCTAGTACTCGGTGGCTTATTGGTTTATAGAAAAAGGTATATACAGATTATGAAATTT
ATTAATTGAGTAATAGTGCATGTATTGGCTTGGTGCAGGGAAAGAAATTAGTTTTTGTAACAAACAACTCTAGAAAGTCGAGA
AGGCAATACTCAAAGAAATTCAGATCACTTGGACTTGAAGTTACTGAGGTATGTTTTGGTCTGAGCGGAGACTCTTCCAGTAA
TAAATTTGTTTTGTATTGTAATCATTTGTTACTTTGTTGTAGGAAGAGATTTTTACATCATCGTTTGCGGCAGCCATGTTCTT
GAAGTTAAATAATTTTCCTCCAGAAAAGAAGGTATCATAAATTATTGTGATGTTCTTTCCTTTTGTTTTATGTGAATGACTTA
TGATTTCTTCGGCACATGATACTTACTGAAATTATCACCATGAGGAAACAAATTAAGAAAGTGGCACATATGTACGCGGGGAA
TTTGATAGCACAACAGCCTACACATTTACAGTAAGATTGGTCACGCCGAACTAATGAGTAAGGGTTTGCCATAAGACTGGAAC
AGCTATGTGGGAACTATGTGGTTGCGATTTTCACTAGAAGACCAATCAAAAGGCACTTATATGAGCCAATCAACCTTGCTTCA
CATAACAGGAGTGTTGTAGATAGTTTGGGTCCGCTATTTCCTGTATACCTCACTTTATTTATCTTGATTTTTCTTGTGTCTGG
TCTTACTTGAACTGCTTATTTTTCTTTAGGTTTATGTTGTTGGTGAAGATGGAATCTTGGAAGAGCTCAAGTTAGCTGGTTTT
GAATGTTTTGGTGGTCCGGTAAGACTTGCTTATTCTCCTCCCCCAACACACACACACCTTTCTCTCTCCTGATTACTAATCAT
TTAGTTTATAACAACTGTTTTCTGCTGAGGGGATGGAAACCATGTTAAGTGTTCAAGATTTTCTTGAATTTCTATCTGTTATC
CTTTTTCTTAGAGTTCCTTTACTATAATGTGAAGATGCTATGTGGTATACTGAATACTGGTTTGATATAGTTCTAACGGTGCA
TCCGTGCTAATGGATCATTTTTTTCATTTGCGAACTTCTCTATACATTGGCATATTCTAGGTTCTATAATTTTCATAATCATA
GTTGACAATAAAATATAAACATAGTGGATCATTGTATGGGATATTACCTGGTTGATCGGGAGTGCCTACTTCAAGGTATTACA
TGTTACTTCGATGTAGTAGTCACTGTGTGTGTGTGTGTGTGTGTGTGTGTGTGTGTGTGTGTGTGTGTGTGTGTGTGTGTGTG
TGTGTGTGTGTGTGTGTGTGTTGTGCTATATGAAATAAAATAGGCCAGAAACATGAAGTGCATTATAAAAACATGCCTAAGTG
TCCTGAGAGAGCCATAGCATACGGACAGTCAAAAGATTTTTTTTGTATACTTGATGAGGGCTTGAAAACATAAAGATTTTTCA
GATAAGTTATCCCTGTTTTACCTTCCATTTCAACTAATGATTTTTTCTGCAGGAGGATGGCAAGAAAAACATAAAGTTGGAGG
CGGATTTCTACTTTGAACATGACAAAAGTGTAATGTCAAATCTGTTTTTCTGATTTTTGTATCATCTTTCACATAAAATACAA
AAGCTACTCAGCTGTGATCTAAATGAAATTTCTTTTCCAGGTTGGAGCTGTCATTGTTGGACTTGATCAGTACTTCAATTATT
ATAAAATGCAGTTATTGCTCTAACCTTGCTGTCTATAGCCTTACCTTTGGCGTTGCTCTCCATGCTGATCAATCATTGCTTCA
CTTTTGTCATACGTGTATTTTCTTCCGTTTCAATGCAAGTTTCATCTCGCTTCTGGAAAATTGAACGAGATAAAACTACATTA
AGGTCCATTTGATTGATCTTTCTTTTCACCATATTTTTCCGGTATGTTGGATGCTCCACCCTTGGCCCTTGCCTAGGCAGTTG
AGCTTCCTTTGTCACATGATTTAGAACATGGTATATTACATATGATATAGACCGAGCCCTCCCAGATATGCTTGTTGTGCATT
TCGTTTTCATTTCCTTTGGTAACTCGTGTCTCCTTTAAACCAGCGGAGTAAAGTTTCATTGCTTGCTCAGTGTTGGTACCTCT
ATGTTGTTCGCCGTTACTTAAAACGTTTGTTGAAAGAGTACCCGTTATTTAATTCAGACCTAAAATTCAGAAGCTTCTAATTT
GAGATAATCCAGCCAAGCAATTATAATCTGCCTTATTTTTATGCTTTATAAAGACATATCACAGATTCACAGTTCCAGTTTTA
AACTTTTGTCGGCAAGGACCACGTTACCACAATAAAAGTACTCCGTCTGTTTTTTGTAGTAAATGGAAAATAAACTAATTAGC
ATGTACTAGAGTAATATGTTCAAAAAACGGAGTAAGAATTTATTTGAGCATCATTTTTCAACATTAGTTGTGACCGCAGCCCA
TGATTCTGTTTACCTTCTAGATCATTAGTTTGACAATGATCGAGTCATAGCGATGTGCTTGCAGGTATGCAAGAACATGTATT
AGTGAGAACCCTGACTGCCTTTTCATTGCGACCAACCGTGATCCAACCGGGCATATGACATCTGCCCAAGAATGGCCAGGTAT
TGTTAATGTAAAATTTATTGAGAATTTTTTTTTTTAAGTTTCGATCTGTAGCACAGTATAATTGGCAAGTGGTTACCTCAAAC
TTAACAGGAGCTGGAGCTATGGTTGCTGCAGTAAGCTGCTCAGTACAGAAAGAACCCATTGTTGTTGGAAAACCTTCAGGCTT
TTTGATGGACTTCCTCTTGAAAAGGTTTGCGAGTTGTATTATGAGAGAAAAAATATATCATTTATTCATAAGTTCAGGGGATT
GCATGCTTTGTTGATTTTTCAATGGCACAGTATGAGATATAAGGTTTCCCTGCTGCAGCTTCAATCTGGAGACGTCAAGGATG
TGCATGGTTGGTGATAGACTGGACACGGACATACTATTTGGCCAGAACACCGGCTGCAAGACCCTCCTTGTTTTGTCTGGTCA
GAGGCTCCTGCTCTTTCTTGTATCCCGAACCGCTTTCTTTCTTTCTTTCTTTCTTTCTCGTGTTCAATGATGATGATGATGTG
TCTCACGTGCGTGGGATTGCAATTGCAGGTTGCACTGCCTTACCAGAAGTGCAGGATGCTCGTAACAATATCCATCCAGATCT
CTACACAAACAGCGTGTATGATCTAGTTGGGTTACTGCAGAAGTAATTCTCCTTGGCCGCGTTGCAGCGCAATCTCAGCACGC
TTTGGATAGCGCCTATCAGCAATTTATTACGTGGATTACTTGCATGAAGCCATGTGTTCTTGAAACAACAATTTTGAACTTTC
TTTGGGGTATAACCTGTTTGACAGTAACAAAATCTTTCAATTGGATCCTACAAATTAGACCAATTTGACAACCTTGTAAGCGT
GGCTGTAAAGCTTTAAATCTGGCAACTTGATTCCCTTGCTGGAAAATATATTGGTTGGAGTATGGAATTGTACAGAATCGAAT
TGTAATGAAGCAACGTGTTGTACTTTGTTG

Updated expression of Zm00001eb064870_P₀₀₁ to 11% with coding regions underlined and difference from reference in bold with wavy underline

provides a design for a polynucleotide sequence to help identify what needs
DNA editing with CDS trained to a 11% expression design level with putative encoding of a
Zm00001eb064870_P001 variant of the Phosphoglycolate phosphatase protein of Zea mays
SEQ ID NO: 24
AGTTCTTTACCACTACCGGCCCAGACACCTAAACGATGACACAGCGTGCACCCACCACCACCAGCGCCGAGCGCCGGCCGCGA
TACCGTCGTGTCCCCCTCCCCACCGCATTAGCGCCGGCCGACAAAATTACTAGGTGCTTAATTTAACTACTAGTGGCCCTAGG
CGTGAAACTTCACCAGCTCCGCCACTGCCCATGGCCAACGGCCGTCCGGACCCTCGCTGCGTCGTCCTCACCGCCGACACTGC
CCGATCCCTCGTAGATTCCGTCGACGCCTTCCTCTTCGACTGCGATGGTATCTGATTGTTCACCTTTCTCCATTTTGTTTGTC
TGTTTTTTTTTCTTCGCTTCAGATTTTGACCCCTGCTTCGCTCTGACCGGTAATTCTAATTGCTTCTCTGAATTTGTCTAGGG
GTCATTTGGAAGGGAGATAAGCTCATTGAAGGGGTCCCAGAGACGATGGAGCTACTGAGGAAACTGGTATGGGAACATCCCTT
TATTACTATTTAAGTATTGAATTCGTTTCCTCATTTTCAATAGTTCAACTGTCAGGCAAATAAATGCCAAATGATCAAAACAA
GTACATAATTAAGGGCTAGCTTGTTTTGTCGTGCCTTAAAAAAAAAAACTGGTTCTAGATTGATTGGGGCTTTGAAGAATCTG
GTTCTCCAAAGTTGCATCTATTTAAGCTAAAATAGAGACAGGCAGTAAGTGCAACCACACCACACCAGGCACCAGATACCGTA
ATGAAATGAAAATCATTGACACTACTACTACTACATTGCTTTAAAGTTATTCTGGTTTTGTTCATCTACATAACTCCTGTCTG
AAACAAGGATAATAGGCCTATATCTTTTTTCTATGTGAATTCTAGCATGATGAACTCACCTCATTAACCAAGATGTGATCTTT
GTTGCGCCATTATTTAAGACATTGCTCTAGTACTCGGTGGCTTATTGGTTTATAGAAAAAGGTATATACAGATTATGAAATTT
ATTAATTGAGTAATAGTGCATGTATTGGCTTGGTGCAGGGAAAGAAATTAGTTTTTGTAACAAACAACTCTAGAAAGTCGAGA
AGGCAATACTCAAAGAAATTCAGATCACTTGGACTTGAAGTTACTGAGGTATGTTTTGGTCTGAGCGGAGACTCTTCCAGTAA
TAAATTTGTTTTGTATTGTAATCATTTGTTACTTTGTTGTAGGAAGAGATTTTTACATCATCGTTTGCGGCAGCCATGTTCTT
GAAGTTAAATAATTTTCCTCCAGAAAAGAAGGTATCATAAATTATTGTGATGTTCTTTCCTTTTGTTTTATGTGAATGACTTA
TGATTTCTTCGGCACATGATACTTACTGAAATTATCACCATGAGGAAACAAATTAAGAAAGTGGCACATATGTACGCGGGGAA
TTTGATAGCACAACAGCCTACACATTTACAGTAAGATTGGTCACGCCGAACTAATGAGTAAGGGTTTGCCATAAGACTGGAAC
AGCTATGTGGGAACTATGTGGTTGCGATTTTCACTAGAAGACCAATCAAAAGGCACTTATATGAGCCAATCAACCTTGCTTCA
CATAACAGGAGTGTTGTAGATAGTTTGGGTCCGCTATTTCCTGTATACCTCACTTTATTTATCTTGATTTTTCTTGTGTCTGG
TCTTACTTGAACTGCTTATTTTTCTTTAGGTTTATGTTGTTGGTGAAGATGGAATCTTGGAAGAACTAAAACTAGCAGGATT
CGAATGCTTCGGAGGACCAGTAAGACTTGCTTATTCTCCTCCCCCAACACACACACACCTTTCTCTCTCCTGATTACTAATC
ATTTAGTTTATAACAACTGTTTTCTGCTGAGGGGATGGAAACCATGTTAAGTGTTCAAGATTTTCTTGAATTTCTATCTGTTA

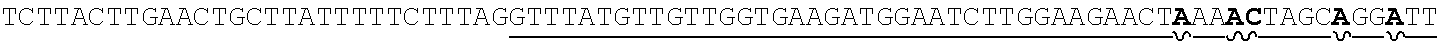

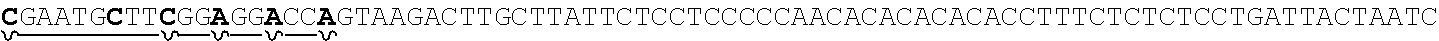

TAGTTGACAATAAAATATAAACATAGTGGATCATTGTATGGGATATTACCTGGTTGATCGGGAGTGCCTACTTCAAGGTATTA
CATGTTACTTCGATGTAGTAGTCACTGTGTGTGTGTGTGTGTGTGTGTGTGTGTGTGTGTGTGTGTGTGTGTGTGTGTGTGTG
TGTGTGTGTGTGTGTGTGTGTGTTGTGCTATATGAAATAAAATAGGCCAGAAACATGAAGTGCATTATAAAAACATGCCTAAG
TGTCCTGAGAGAGCCATAGCATACGGACAGTCAAAAGATTTTTTTTGTATACTTGATGAGGGCTTGAAAACATAAAGATTTTT

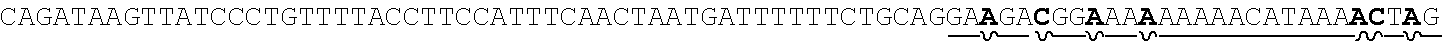

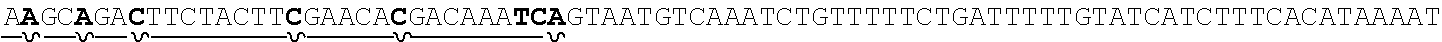

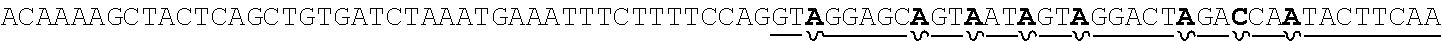

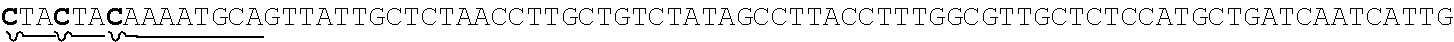

CTTCACTTTTGTCATACGTGTATTTTCTTCCGTTTCAATGCAAGTTTCATCTCGCTTCTGGAAAATTGAACGAGATAAAACTA
CATTAAGGTCCATTTGATTGATCTTTCTTTTCACCATATTTTTCCGGTATGTTGGATGCTCCACCCTTGGCCCTTGCCTAGGC
AGTTGAGCTTCCTTTGTCACATGATTTAGAACATGGTATATTACATATGATATAGACCGAGCCCTCCCAGATATGCTTGTTGT
GCATTTCGTTTTCATTTCCTTTGGTAACTCGTGTCTCCTTTAAACCAGCGGAGTAAAGTTTCATTGCTTGCTCAGTGTTGGTA
CCTCTATGTTGTTCGCCGTTACTTAAAACGTTTGTTGAAAGAGTACCCGTTATTTAATTCAGACCTAAAATTCAGAAGCTTCT
AATTTGAGATAATCCAGCCAAGCAATTATAATCTGCCTTATTTTTATGCTTTATAAAGACATATCACAGATTCACAGTTCCAG
TTTTAAACTTTTGTCGGCAAGGACCACGTTACCACAATAAAAGTACTCCGTCTGTTTTTTGTAGTAAATGGAAAATAAACTAA
TTAGCATGTACTAGAGTAATATGTTCAAAAAACGGAGTAAGAATTTATTTGAGCATCATTTTTCAACATTAGTTGTGACCGCA

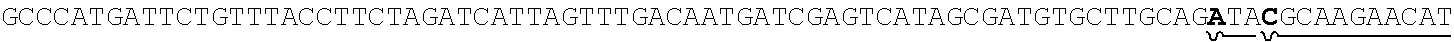

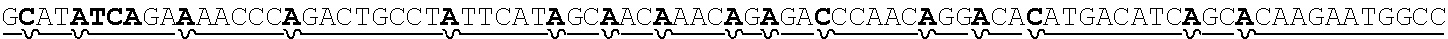

AGGTATTGTTAATGTAAAATTTATTGAGAATTTTTTTTTTTAAGTTTCGATCTGTAGCACAGTATAATTGGCAAGTGGTTACC

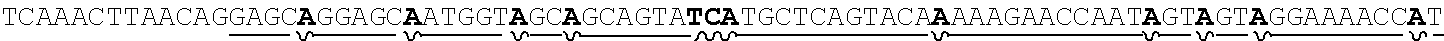

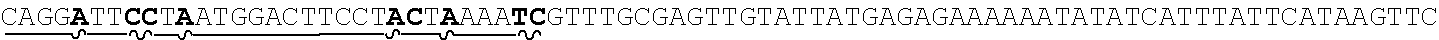

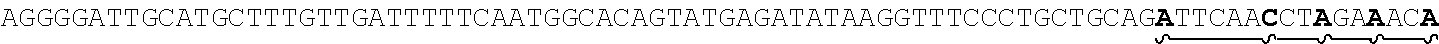

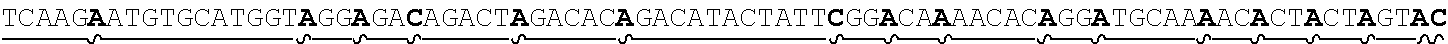

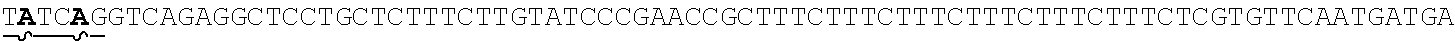

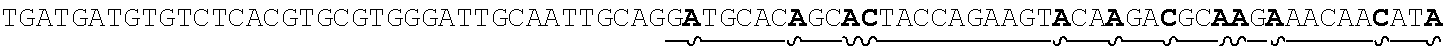

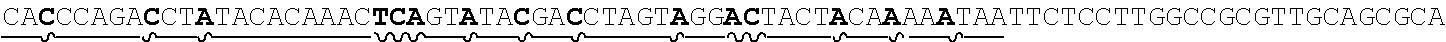

ATCTCAGCACGCTTTGGATAGCGCCTATCAGCAATTTATTACGTGGATTACTTGCATGAAGCCATGTGTTCTTGAAACAACAA
TTTTGAACTTTCTTTGGGGTATAACCTGTTTGACAGTAACAAAATCTTTCAATTGGATCCTACAAATTAGACCAATTTGACAA
CCTTGTAAGCGTGGCTGTAAAGCTTTAAATCTGGCAACTTGATTCCCTTGCTGGAAAATATATTGGTTGGAGTATGGAATTGT
ACAGAATCGAATTGTAATGAAGCAACGTGTTGTACTTTGTTG

Original expression of Zm00001eb299920_T001 is @ 59.5%, with coding regions underlined:

SEQ ID NO: 25 provides the polynucleotide sequence of the coding strand
of the DNA region transcribed then spliced to the Zm00001eb299920_T001
transcript with CDS encoding of the Zm00001eb299920_P001
variant of a Glyoxylate reductase protein of Zea mays

CCAGGGGCCAACACAGCACCGACACCTAAAAAAAATGGTCGTGCACAGGAGCGTTGCACTTGCAGCTGATCTCATCTGATCCG
ATCAATCAACGTGACATGATCTGCAGACGGTCGAATGATGCTTCAGTAAAATATTTTTATAAGTTTACGGTCGGTCCCTCAAA
TCATCATCATCACCAATATTATAATTGTTACCAACAGCTCGCCTGCCCACCAAATCGGCAAATCCTGGCGTACAGGAGGACGA
CCCAACCCAAGCGGCACACAAGATCCAAGAGGATGCCGCCGCCGCCGCCGCTCGTGCTGCTGGCGAAGCCGCTGTTCCCGAAC
TTCGCGGCGGCGCTGGAGGGCCGGTACCGCCTCGTCCTGGCCGGGGACGCCGACGCGGCCACGGCGGCGGAGGCCCGGGTCCT
GCTCGTGCCGGGGCTCGTGGCCGTGACAGCCGAGCTCGTCGACCGGCTCCCGGCGCTGGAGCTCGTGGCCGCGACCTCCGTCG
GGCTGGACCACGTGGACCTCCGTGCCTGCCGCCGCCGCGGGCTCGCCGTCACCAACGCCGGCGCGGCCTTCTCCGTCGACTCC
GCCGACTACGCCGTCGGCCTCGTCGTCGCCGTGCTGCGCAGGGTCGCCGCCGCCGAGGCGCATCTCCGGCGCGGCGGGTGGGC
TACCGATGGCGAGTACCCGCTCACCACCAAGGTATAGCAGCATACTGAGAATTGCCACCCAAAAAAAACAAACAAACAGAAGA
ACAGAATTCGACATGTCTCGGACTCTCGGTTGAATTAATTTGCAGGTGAGCGGCAAGCGGGTGGGGATCGTGGGGCTCGGCAG
CATCGGCTCGCTGGTGGCGCGGCGCCTGGCCGCCATGGGGTGCCGCGTCGCCTACCACTCCCGCGCGCCGAAGCCGTCGTGCT
CGTGCCCCTACGCCTTCTTCCCCACGGCGCGCGCCCTGGCCTTGGCCAGCGACGTGCTGGTGCTGTCGTGCGCGCTCACGGAG
GAGACGCGGCGCGTGGTGGGCCGCGAGGTGCTGGAGGCGCTGGGACAGGGCGGCGTGCTGGTCAACGTCGGCCGCGGCGGCCT
GGTCGACGAGCCGGAGCTGGTGCGGTGCCTGCGGGAGGGCGTCATCGGCGGCGCGGGGCTGGACGTGTTCGAGGACGAGCCCG
ACGTGCCCGCGGAGCTCCTCGCCATGGACAACGTCGTGCTGTCGCCCCACAGGGCCGTGCTCACGCCGGAGTCCATGCGCGGG
TTGCTGGATGTCGTCGCGGGAAACCTCGACGCCTTCTTCGCCGGCAGGCCGCTGCTCAGCCCCGTGTCGCTGTGAATTCTGAA
CCACATGTAGGATAGATACGTCAAGTCGTCAAGTTCCATTCCATCCAAGAAGCATCAACCAGACAACCAGTGTGTGAGACTCA
CCACGAACAATACGGAAGAACACTCAGAATTGGTAGGGTGGTTCGGGTTTCTTACTTTCTGTTCCAAGTTCATCATCCAAAAG
CCAAATTTCAAAATGACAACAGTTCGTCTTTTATACTTGCTACTGGAAAACCAAAACGAATTGGAATATATTACAGCCAGTTG
GCTCCA

Updated expression of Zm00001eb299920_P₀₀₁ to 74%, with coding regions underlined and differences from reference in bold with wavy underline:

provides a design for a polynucleotide sequence to help identify what needs
DNA editing with CDS trained to a 74% expression design level with putative encoding
of a Zm00001eb299920_P001 variant of the Glyoxylate reductase protein of Zea mays
SEQ ID NO: 26
CCAGGGGCCAACACAGCACCGACACCTAAAAAAAATGGTCGTGCACAGGAGCGTTGCACTTGCAGCTGATCTCATCTGATCCG
ATCAATCAACGTGACATGATCTGCAGACGGTCGAATGATGCTTCAGTAAAATATTTTTATAAGTTTACGGTCGGTCCCTCAAA
TCATCATCATCACCAATATTATAATTGTTACCAACAGCTCGCCTGCCCACCAAATCGGCAAATCCTGGCGTACAGGAGGACGA

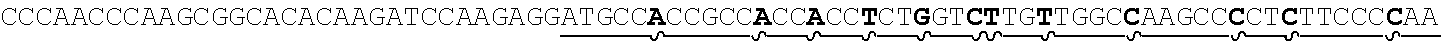

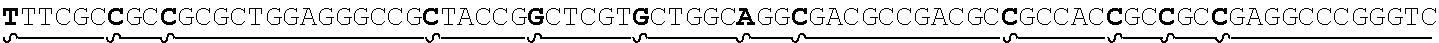

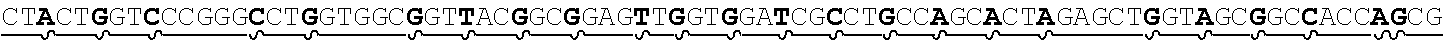

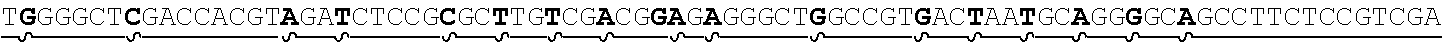

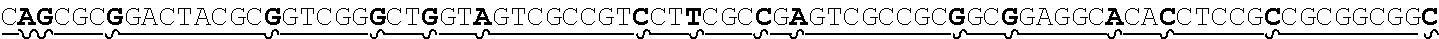

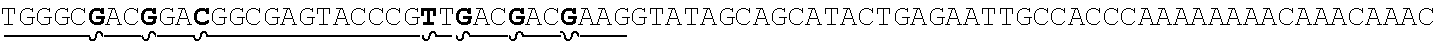

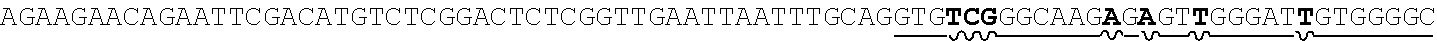

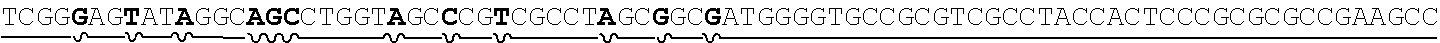

GTCGTGCTCGTGCCCCTACGCCTTCTTCCCCACGGCGCGCGCCCTGGCCTTGGCCAGCGACGTGCTGGTGCTGTCGTGCGCGC
TCACGGAGGAGACGCGGCGCGTGGTGGGCCGCGAGGTGCTGGAGGCGCTGGGACAGGGCGGCGTGCTGGTCAACGTCGGCCGC
GGCGGCCTGGTCGACGAGCCGGAGCTGGTGCGGTGCCTGCGGGAGGGCGTCATCGGCGGCGCGGGGCTGGACGTGTTCGAGGA
CGAGCCCGACGTGCCCGCGGAGCTCCTCGCCATGGACAACGTCGTGCTGTCGCCCCACAGGGCCGTGCTCACGCCGGAGTCCA
TGCGCGGGTTGCTGGATGTCGTCGCGGGAAACCTCGACGCCTTCTTCGCCGGCAGGCCGCTGCTCAGCCCCGTGTCGCTGTGA
ATTCTGAACCACATGTAGGATAGATACGTCAAGTCGTCAAGTTCCATTCCATCCAAGAAGCATCAACCAGACAACCAGTGTGT
GAGACTCACCACGAACAATACGGAAGAACACTCAGAATTGGTAGGGTGGTTCGGGTTTCTTACTTTCTGTTCCAAGTTCATCA
TCCAAAAGCCAAATTTCAAAATGACAACAGTTCGTCTTTTATACTTGCTACTGGAAAACCAAAACGAATTGGAATATATTACA
GCCAGTTGGCTCCA

### E. Example 5: Gene Training Towards Increased Root Growth

In this example, we focus on precisely increasing expression of an existing gene in a maize inbred toward increasing root growth. In this example, the Zm00001eb234360_T001 splice sequence from the B73 reference appears homologous to RITF1 (i.e. Arabidopsis root growth factor inducible transcription factor) and is trained from 36.3% design level to a higher relative expression design level of 40.2% towards increasing root growth as one example towards carbon capture.

RITF1_ARATH protein sequence:

SEQ ID NO: 27 provides the amino acid sequence
of the RITF41 protein of Arabidopsis thaliana

MGIQKPAWLDALYAEKFFVGCPYHETAKKNERNVCCLDCCTSLCPHCVPS
HRFHRLLQVRRYVYHDVVRLEDLQKLIDCSNVQAYTINSAKVVFIKKRPQ
NRQFKGAGNYCTSCDRSLQEPYIHCSLGCKVDFVMKRYRDITPFLKPCHT
LTLGPDYIIPQDLLTDDEVAAYETPRSTVVDGDESMSWSSASSDNNNAGA
AAAYAATTTHVVRKKRTGFCLCAKSANSYKEVSEDPDDISACINRRKGVP
QRSPLCC

Zm00001eb234360_P₀₀₁ protein sequence (putative root growth factor inducible transcription factor):

SEQ ID NO: 28 provides the amino acid sequence of
the Zm00001eb234360_P001 variant of a putative
homolog of Arabidopsis thaliana RITF1 protein
from Zea mays

MIMAMWKPGWLEALDTQKFFVACSFHEHAKKNEKNICCLDCCTSICPHCV
AAHRAHRLLQVRRYVYHDVVRLEDLEKLIDCSSVQSYTINSSKVVFLKKR
PQNRQFKGSGNICTSCDRSLQEPYFHCSLDCKVEYILRQKKKLSAYLRPC
KTLQLGPDFFIPHDADDDTTHSTLVDVDEPMGSSDSENLSVPCTNFVRKK
RSGPYICARSANRVSEEDMATNMSRRKGVPQRSPLC

Example Reference CDS RNA Sequence (Reference Level, −MFE=239; [0-1] design level=36.3%)

SEQ ID NO: 29 provides the nucleic acid sequence
of the Zm00001eb234360_T001 CDS encoding of the
Zm00001eb234360_P001 variant of a putative
homolog of Arabidopsis thaliana RITF1 protein
from Zea mays
AUGAUCAUGGCAAUGUGGAAGCCAGGAUGGCUAGAGGCCCUUGACACACA
GAAGUUCUUCGUAGCAUGCUCUUUCCAUGAGCAUGCCAAGAAGAACGAGA
AGAACAUCUGUUGCCUUGACUGCUGCACUAGCAUCUGCCCACACUGUGUG
GCAGCACACCGUGCACACAGGCUCCUGCAGGUGCGGCGAUACGUCUACCA
UGACGUUGUCCGGCUGGAGGACCUGGAGAAGCUUAUUGAUUGCUCUAGUG
UUCAGUCUUAUACUAUUAACAGCUCUAAGGUUGUUUUCCUGAAGAAGAGA
CCACAGAAUAGGCAAUUCAAGGGUUCAGGGAAUAUCUGCACCUCCUGCGA
CAGGAGCCUUCAAGAACCGUAUUUCCACUGCUCUCUGGAUUGCAAGGUAG
AGUAUAUACUACGACAGAAGAAAAAAUUGUCAGCAUAUUUGCGCCCAUGC
AAGACCUUGCAGCUUGGCCCUGAUUUCUUCAUUCCUCAUGAUGCUGAUGA
CGACACAACUCACUCAACCCUUGUUGAUGUUGAUGAGCCCAUGGGAUCAU
CGGACUCGGAGAAUUUGAGUGUGCCGUGCACAAAUUUUGUUCGGAAAAAA
CGGAGUGGACCAUAUAUUUGUGCACGGUCUGCAAACAGAGUGUCUGAAGA
AGACAUGGCCACAAAUAUGAGCAGAAGGAAAGGGGUUCCUCAGAGAUCGC
CUUUGUGCUAA

Example Trained CDS RNA Sequence (−MFE=254, [0-1] design level=40.2%), with differences from reference underlined

SEQ ID NO: 30 provides a design for a CDS sequence
trained to a 40.2% expression design level
encoding of a Zm00001eb234360_P001 variant of a
putative homolog of Arabidopsis thaliana RITF1
protein of Zea mays

AUGAUCAUGGCCAUGUGGAAGCCGGGCUGGCUGGAGGCUCUGGACACACA
AAAGUUCUUCGUGGCGUGCUCGUUCCACGAGCACGCCAAGAAGAACGAGA
AGAACAUAUGUUGCCUAGAUUGCUGCACCUCGAUCUGCCCGCAUUGUGUG
GCCGCUCAUCGGGCGCACCGCCUCCUCCAGGUGCGCCGCUACGUGUACCA
CGAUGUGGUGCGGCUGGAGGACCUGGAGAAGCUUAUUGAUUGCUCUAGUG
UUCAGUCUUAUACUAUUAACAGCUCUAAGGUUGUUUUCCUGAAGAAGAGA
CCACAGAAUAGGCAAUUCAAGGGUUCAGGGAAUAUCUGCACCUCCUGCGA
CAGGAGCCUUCAAGAACCGUAUUUCCACUGCUCUCUGGAUUGCAAGGUAG
AGUAUAUACUACGACAGAAGAAAAAAUUGUCAGCAUAUUUGCGCCCAUGC
AAGACCUUGCAGCUUGGCCCUGAUUUCUUCAUUCCUCAUGAUGCUGAUGA
CGACACAACUCACUCAACCCUUGUUGAUGUUGAUGAGCCCAUGGGAUCAU
CGGACUCGGAGAAUUUGAGUGUGCCGUGCACAAAUUUUGUUCGGAAAAAA
CGGAGUGGACCAUAUAUUUGUGCACGGUCUGCAAACAGAGUGUCUGAAGA
AGACAUGGCCACAAAUAUGAGCAGAAGGAAAGGGGUUCCUCAGAGAUCGC
CUUUGUGCUAA

Example Trained Coding Strand DNA Excerpt w/coding region underlined and differences from reference highlighted in bold with wavy underline (Template Strand putatively transcribed to primary transcript) wherein the differences can be implemented by way of gene editing, modification, or replacement.

provides a design for a polynucleotide sequence to help identify what needs
DNA editing with CDS trained to a 40.2% expression design level with putative encoding of a
Zm00001eb234360_P001 variant of a putative homolog of Arabidopsis thaliana RITF1 protein
of Zea mays
SEQ ID NO: 31
CTTCTAACAAGCATACTACACACCCCTCCTCCCTCCCCATCCTTCATAAGCTCTCATACGTCTCTCTCTCTCTCCCTTCTTCA
AAACACCCCACCCCATCCAAAACCTAGAACACACACAACACCAGAGGGCTTTACAACCGCGCACATGATCATGGTAAGGACTC
TAACCTTCATGTTTTCATGTGAATTCTCGTATGAAATCGCCATTTATTGAGCAAAAAAAAATATATGGAATTGGGCAACACCA

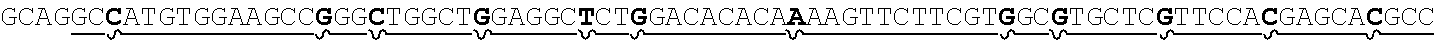

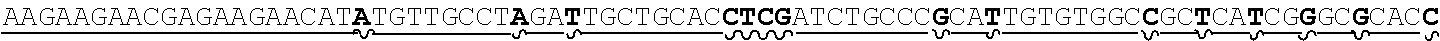

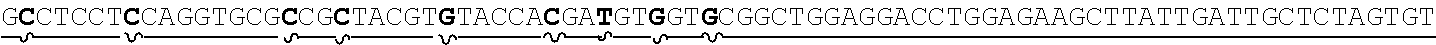

TCAGGTATATCAATTATGCACAGTGCATGCATGTGACTCTGGTGTTGTTCTGAGGTTCAAGATGCAAGAAGGCTTATAATTCT
GTATCAACGTGGTGCAGTCTTATACTATTAACAGCTCTAAGGTTGTTTTCCTGAAGAAGAGACCACAGAATAGGCAATTCAAG
GGTTCAGGGAATATCTGCACCTCCTGCGACAGGAGCCTTCAAGAACCGTATTTCCACTGCTCTCTGGATTGCAAGGTGATTAA
CTCGAACTGATATGCTATGGTTATCCTACTATGTTTCAGAAAATAAAATTCATGCTAAATGAGGGGAGCAGAATAATTATGAT
CTAAAAGGTCGTTAGGAAGAGCAAGTATTTCGAACTTGTTTGTCATGCAATATTCATGCCAGTTATAAAAAAATTTGTTTCAT
AGTATGCATCATTTTCAGGTAGAGTATATACTACGACAGAAGAAAAAATTGTCAGCATATTTGCGCCCATGCAAGACCTTGCA
GCTTGGCCCTGATTTCTTCATTCCTCATGATGCTGATGACGACACAACTCACTCAACCCTTGTTGATGTTGATGAGCCCATGG
GATCATCGGACTCGGAGAATTTGAGTGTGCCGTGCACAAATTTTGTTCGGAAAAAACGGAGTGGACCATATATTTGTGCACGG
TCTGCAAACAGAGTGTCTGAAGAAGACATGGCCACAAATATGAGCAGAAGGAAAGGGGTTCCTCAGAGATCGCCTTTGTGCTA
ATAAATTCAATAGTTAATTATTTTCCCTTTTCTTTACTTCTTTCATGTTCTTTATCTCTTGGACTAAACTGGTGCAACAATGA
TAGAATTTTGTCTCAGTACTACCATGTGATTAAAATACATCACTAGAGGCAATTGTACTCAATTCTTCAAGTTGGTGCACCAG
TTTATATATATGTATGCTTTAACATAGTTCATGTTATCCCCTTTT

Although introns are said may be necessary for mobility within a cell, the introns need not be preserved if they indeed serve no function. The below is a version of the trained coding strand DNA excerpt without introns for an example gene replacement with coding region underlined and coding region differences highlighted in bold with wavy underline (Template Strand putatively transcribed to primary transcript).

provides a design for a polynucleotide sequence to help identify what needs
DNA editing in a gene replacement with intron sequences excluded with CDS trained
to a 40.2%expression design level encoding of a Zm00001eb234360_P001 variant of a
putative homolog of Arabidopsis thaliana RITF1 protein of Zea mays
SEQ ID NO: 32
CTTCTAACAAGCATACTACACACCCCTCCTCCCTCCCCATCCTTCATAAGCTCTCATACGTCTCTCTCTCTCTCCCTTCTTCA

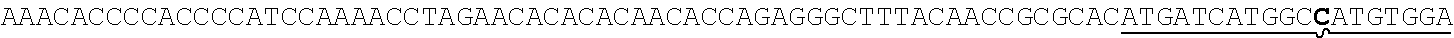

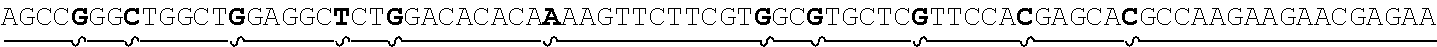

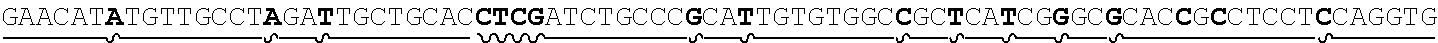

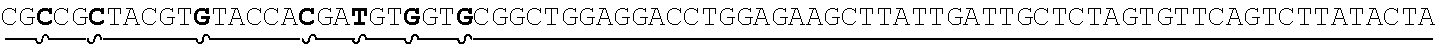

TTAACAGCTCTAAGGTTGTTTTCCTGAAGAAGAGACCACAGAATAGGCAATTCAAGGGTTCAGGGAATATCTGCACCTCCTGC
GACAGGAGCCTTCAAGAACCGTATTTCCACTGCTCTCTGGATTGCAAGGTAGAGTATATACTACGACAGAAGAAAAAATTGTC
AGCATATTTGCGCCCATGCAAGACCTTGCAGCTTGGCCCTGATTTCTTCATTCCTCATGATGCTGATGACGACACAACTCACT
CAACCCTTGTTGATGTTGATGAGCCCATGGGATCATCGGACTCGGAGAATTTGAGTGTGCCGTGCACAAATTTTGTTCGGAAA
AAACGGAGTGGACCATATATTTGTGCACGGTCTGCAAACAGAGTGTCTGAAGAAGACATGGCCACAAATATGAGCAGAAGGAA
AGGGGTTCCTCAGAGATCGCCTTTGTGCTAATAAATTCAATAGTTAATTATTTTCCCTTTTCTTTACTTCTTTCATGTTCTTT
ATCTCTTGGACTAAACTGGTGCAACAATGATAGAATTTTGTCTCAGTACTACCATGTGATTAAAATACATCACTAGAGGCAAT
TGTACTCAATTCTTCAAGTTGGTGCACCAGTTTATATATATGTATGCTTTAACATAGTTCATGTTATCCCCTTTT

### F. Example 6: Gene Training Towards Antibacterial, Antifungal, & Pest Resistance

In this example, we focus on precisely increasing expression of an existing gene in a maize inbred toward increasing resilience to biotic stressors and protection of yield. Zm00001eb165590_P₀₀₁ is putatively the BX8 enzyme (DIMBOA UDP-glucosyltransferase) that is said can stabilize DIMBOA and similar anti-biotics into DIMBOA glucosides and similar in grasses/crops such as maize. In this example, the Zm00001eb165590_T001 splice sequence from the B73 reference is trained from 56.3% to a higher relative expression design level of 65% towards allowing the increased presence of DIMBOA-associated anti-biotics and resistance to bacteria, fungi, and some insects.

Zm00001eb165590_P₀₀₁ protein sequence (putative BX8 DIMBOA UDP-glucosyltransferase):

SEQ ID NO: 33 provides the amino acid sequence of
the Zm00001eb165590_P001 variant of a BX8 DIMBOA
UDP-glucosyltransferase protein of Zea mays

MIMAMWKPGWLEALDTQKFFVACSFHEHAKKNEKNICCLDCCTSICPHCV
AAHRAHRLLQVRRYVYHDVVRLEDLEKLIDCSSVQSYTINSSKVVFLKKR
PQNRQFKGSGNICTSCDRSLQEPYFHCSLDCKVEYILRQKKKLSAYLRPC
KTLQLGPDFFIPHDADDDTTHSTLVDVDEPMGSSDSENLSVPCTNFVRKK
RSGPYICARSANRVSEEDMATNMSRRKGVPQRSPLC

Example Reference CDS RNA Sequence (Reference Level, −MFE=768; [0-1] design level=56.3%):

SEQ ID NO: 34 provides the nucleic acid sequence of the Zm00001eb165590_T001 CDS
encoding of the Zm00001eb165590_P001 variant of a BX8 DIMBOA UDP-glucosyl-
transferase protein (also annotated as a glycosyltransferase) from Zea mays

AUGCACACGGACGACGUCUUUCUGGCAUCGCUACUACCAGACAGAGAGGACAGGGGAGGUCAGACUCAGAAACGCUCGCUCAC
GACGGCAGCAGCCAUGGCAGCAUCGUGCGGCGGCCGCGUGGUGGUCUUCCCGUUCCCGUUCCAGGGCCACUUCAACCCGGUGA
UGCGCCUGGCCCGCGCGCUGCACGCCCGGGGCGUCGGGAUCACCGUGUUCCACACCGCCGGCGCGCGGGCGCCGGACCCGGCC
GACUACCCCGCCGACUACCGCUUCGUGCCCGUGCCCGUGGAGGUGGCCCCGGAGCUGAUGGCGUCCGAGGACAUCGCCGCCAU
CGUCACGGCGCUCAACGCCGCCUGCGAGGCGCCCUUCCGGGACCGCCUCUCGGCGCUGCUCUCCGCCGCCGACGGCGAGGCGG
GGGAGGCGGGGGGCCGCGUCCGCUGCGUCCUCACCGACGUCAGCUGGGACGCCGUGCUGUCGGCGGCCCGCGGCCUCGGCGUG
CCCGCGCUCGGCGUCAUGACGGCCAGCGCCGCCACGUUCCGCGUCUACAUGGCGUACCGCACCUUGGUCGACAAGGGAUACCU
GCCGGUGAGAGAGGAGCGCAAGGACGACGCGGUCGCCGAGCUACCCCCGUACCGCGUGAAGGACCUGCUGCGGCACGAGACGU
GCGACCUGGAGGAGUUCGCGGACCUGCUGGGCCGCGUGGUCGCGGCGGCGCGGCUGUCCUCGGGGCUCAUCUUCCACACGUUC
CCCUUCAUCGAGGCCGGCACGCUGGGCGAGAUCCGGGACGACAUGUCGGUGCCGGUGUACGCCGUGGCGCCGCUCAACAAGCU
GGUGCCGGCGGCCACGGCCAGCCUGCACGGCGAGGUGCAGGCGGACCGGGGCUGCCUGCGCUGGCUGGACGCGCAGCGGGCGC
GCUCCGUGCUGUACGUGAGCUUCGGGAGCAUGGCGGCCAUGGACCCGCACGAGUUCGUGGAGCUGGCGUGGGGGCUGGCCGAC
GCCGGCCGCCCCUUCGUGUGGGUGGUCCGGCCCAACCUCAUCCGCGGCUUCGAGUCGGGCGCGCUGCCCGACGGCGUGGAGGA
CCGGGUGCGCGGCCGCGGCGUCGUCGUCAGCUGGGCGCCGCAGGAGGAGGUGCUCGCGCACCCGGCCGUGGGCGGCUUCUUCA
CCCACUGCGGCUGGAACUCCACCGUGGAGGCCGUGUCGGAGGGCGUGCCCAUGAUCUGCCACCCGCGCCACGGGGACCAGUAC
GGCAACGCGAGGUACGUGUGCCACGUCUGGAAGGUGGGCACGGAGGUCGCCGGGGACCAGCUGGAGAGAGGGGAGAUCAAGGC
CGCCAUCGACAGGCUCAUGGGCGGCAGCGAGGAAGGGGAGGGCAUCAGGAAGAGGAUGAACGAGCUCAAGAUCGCUGCGGACA
AGGGCAUCGAUGAAUCUGCUGGGUCGGAUUUAACUAAUUUGGUUCAUCUCAUAAACUCCUACUGA

Example Trained CDS RNA Sequence (−MFE=853, [0-1] design level=65.0%), with differences from reference underlined:

SEQ ID NO: 35 provides a design for a nucleic acid CDS sequence trained to a 65.0%
expression design level encoding of a Zm00001eb165590_P001 variant of a BX8 DIMBOA
UDP-glucosyltransferase protein of Zea mays

AUGCACACCGACGACGUGUUCCUCGCAAGCUUGCUGCCAGAUCGCGAGGACCGCGGUGGUCAGACUCAAAAACGGAGUCUGAC
CACCGCGGCCGCGAUGGCAGCAAGCUGCGGGGGACGCGUCGUGGUGUUCCCCUUCCCCUUCCAGGGCCACUUUAAUCCCGUCA
UGCGCCUAGCGCGGGCACUCCAUGCCCGCGGGGUGGGCAUCACCGUGUUCCACACCGCCGGUGCGCGCGCACCGGACCCGGCC
GAUUACCCCGCCGAUUACCGGUUUGUCCCCGUCCCCGUGGAGGUAGCGCCGGAAUUGAUGGCCAGCGAGGAUAUCGCGGCCAU
CGUGACGGCCCUGAACGCGGCCUGCGAGGCCCCGUUCAGGGACCGUCUUUCGGCGCUGCUCUCCGCCGCCGACGGCGAGGCGG
GGGAGGCGGGGGGCCGCGUCCGCUGCGUCCUCACCGACGUCAGCUGGGACGCCGUGCUGUCGGCGGCCCGCGGCCUCGGCGUG
CCCGCGCUCGGCGUCAUGACGGCCAGCGCCGCCACGUUCCGCGUCUACAUGGCGUACCGCACCUUGGUCGACAAGGGAUACCU
GCCGGUGAGAGAGGAGCGCAAGGACGACGCGGUCGCCGAGCUACCCCCGUACCGCGUGAAGGACCUGCUGCGGCACGAGACGU
GCGACCUGGAGGAGUUCGCGGACCUGCUGGGCCGCGUGGUCGCGGCGGCGCGGCUGUCCUCGGGGCUCAUCUUCCACACGUUC
CCCUUCAUCGAGGCCGGCACGCUGGGCGAGAUCCGGGACGACAUGUCGGUGCCGGUGUACGCCGUGGCGCCGCUCAACAAGCU
GGUGCCGGCGGCCACGGCCAGCCUGCACGGCGAGGUGCAGGCGGACCGGGGCUGCCUGCGCUGGCUGGACGCGCAGCGGGCGC
GCUCCGUGCUGUACGUGAGCUUCGGGAGCAUGGCGGCCAUGGACCCGCACGAGUUCGUGGAGCUGGCGUGGGGGCUGGCCGAC
GCCGGCCGCCCCUUCGUGUGGGUGGUCCGGCCCAACCUCAUCCGCGGCUUCGAGUCGGGCGCGCUGCCCGACGGCGUGGAGGA
CCGGGUGCGCGGCCGCGGCGUCGUCGUCAGCUGGGCGCCGCAGGAGGAGGUGCUCGCGCACCCGGCCGUGGGCGGCUUCUUCA
CCCACUGCGGCUGGAACUCCACCGUGGAGGCCGUGUCGGAGGGCGUGCCCAUGAUCUGCCACCCGCGCCACGGGGACCAGUAC
GGCAACGCGAGGUACGUGUGCCACGUCUGGAAGGUGGGCACGGAGGUCGCCGGGGACCAGCUGGAGAGAGGGGAGAUCAAGGC
CGCCAUCGACAGGCUCAUGGGCGGCAGCGAGGAAGGGGAGGGCAUCAGGAAGAGGAUGAACGAGCUCAAGAUCGCUGCGGACA
AGGGCAUCGAUGAAUCUGCUGGGUCGGAUUUAACUAAUUUGGUUCAUCUCAUAAACUCCUACUGA

Example Trained Coding Strand DNA Excerpt w/coding regions underlined and example differences highlighted in bold with wavy underline (Template Strand putatively transcribed to primary transcript):

provides a design for a polynucleotide sequence to help identify what needs
DNA editing with CDS trained to a 65.0% expression design level with putative
encoding of a Zm00001eb165590_P001 variant of a BX8 DIMBOA UDP-glucosyltransferase
protein of Zea mays
SEQ ID NO: 36

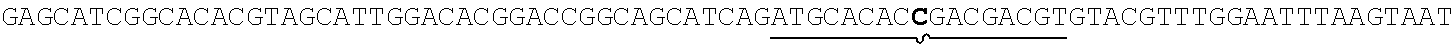

TTTTTTAGTGGAAGAATTATATCACCCACCACCTGCTGTTGGATCGAGCGTGTGCGTGTCGGTGTCTGTGTCTCATCATCGCC

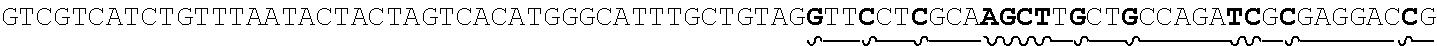

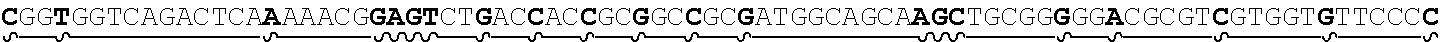

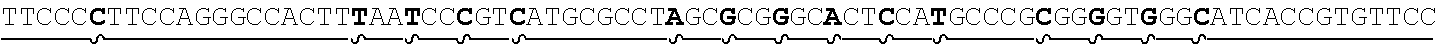

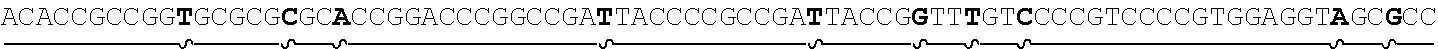

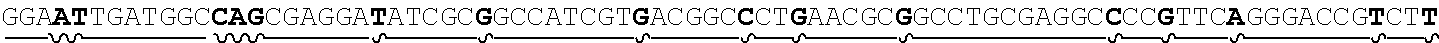

TCGGCGCTGCTCTCCGCCGCCGACGGCGAGGCGGGGGAGGCGGGGGGCCGCGTCCGCTGCGTCCTCACCGACGTCAGCTGGGA
CGCCGTGCTGTCGGCGGCCCGCGGCCTCGGCGTGCCCGCGCTCGGCGTCATGACGGCCAGCGCCGCCACGTTCCGCGTCTACA
TGGCGTACCGCACCTTGGTCGACAAGGGATACCTGCCGGTGAGAGGTGAGTCCGTCCAAGTCGATTTCGTCGGCGGCGGCGAG
GTTAGAGTTAGATTAGATCTGCCTACTTGCTTATACATAATGGTTTGGATTGGATGCGTGAATCGATCTAATCAAATCAGAGG
AGCGCAAGGACGACGCGGTCGCCGAGCTACCCCCGTACCGCGTGAAGGACCTGCTGCGGCACGAGACGTGCGACCTGGAGGAG
TTCGCGGACCTGCTGGGCCGCGTGGTCGCGGCGGCGCGGCTGTCCTCGGGGCTCATCTTCCACACGTTCCCCTTCATCGAGGC
CGGCACGCTGGGCGAGATCCGGGACGACATGTCGGTGCCGGTGTACGCCGTGGCGCCGCTCAACAAGCTGGTGCCGGCGGCCA
CGGCCAGCCTGCACGGCGAGGTGCAGGCGGACCGGGGCTGCCTGCGCTGGCTGGACGCGCAGCGGGCGCGCTCCGTGCTGTAC
GTGAGCTTCGGGAGCATGGCGGCCATGGACCCGCACGAGTTCGTGGAGCTGGCGTGGGGGCTGGCCGACGCCGGCCGCCCCTT
CGTGTGGGTGGTCCGGCCCAACCTCATCCGCGGCTTCGAGTCGGGCGCGCTGCCCGACGGCGTGGAGGACCGGGTGCGCGGCC
GCGGCGTCGTCGTCAGCTGGGCGCCGCAGGAGGAGGTGCTCGCGCACCCGGCCGTGGGCGGCTTCTTCACCCACTGCGGCTGG
AACTCCACCGTGGAGGCCGTGTCGGAGGGCGTGCCCATGATCTGCCACCCGCGCCACGGGGACCAGTACGGCAACGCGAGGTA
CGTGTGCCACGTCTGGAAGGTGGGCACGGAGGTCGCCGGGGACCAGCTGGAGAGAGGGGAGATCAAGGCCGCCATCGACAGGC
TCATGGGCGGCAGCGAGGAAGGGGAGGGCATCAGGAAGAGGATGAACGAGCTCAAGATCGCTGCGGACAAGGGCATCGATGAA
TCTGCTGGGTCGGATTTAACTAATTTGGTTCATCTCATAAACTCCTACTGATCGATCCTTGTATTGGGTACCATGTTCCGGAT
GATGAGTTGGAATAAATCAAAACTTGTGTTCCAGTCGATCTACGCTTCTCATCATTCATCATCATCAATGGACAACAAAACCT
ACAGCTACAGGGTTTAGAACATTAACAGACTTGTTTTCGAGCTTCCTCAGGATTCAACCCAGTTGTGCTAGAGCATCGTCTCT
AACAGATTATGGATGTTCTCCTGATATTAGTAACAGATTATGGATGTTCTCCTGATATTA

### G. Example 7: Gene Training Towards Increasing Durability of Herbicide Tolerance

In this example, we focus on precisely increasing expression of two potentially herbicide tolerant acetolactate synthase (ALS) genes (ALS1 and ALS2) with hra-like mutations in a maize inbred toward increasing durability and resilience to a wide range of ALS-inhibiting herbicides and to specific sulfonylurea and triazolopyrimidine herbicides to help with weed control and protection of yield.

Zm00001eb241810_P₀₀₁ is a non-herbicide tolerant ALS1 protein sequence:

SEQ ID NO: 37 provides the amino acid sequence
of the Zm00001eb241810_P001 variant of an
acetolactate synthase 1 protein of Zea mays

MATAAAASTALTGATTAAPKARRRAHLLATRRALAAPIRCSAASPAMPMA
PPATPLRPWGPTDPRKGADILVESLERCGVRDVFAYPGGASMEIHQALTR
SPVIANHLFRHEQGEAFAASGYARSSGRVGVCIATSGPGATNLVSALADA
LLDSVPMVAITGQVPRRMIGTDAFQETPIVEVTRSITKHNYLVLDVDDIP
RVVQEAFFLASSGRPGPVLVDIPKDIQQQMAVPVWDKPMSLPGYIARLPK
PPATELLEQVLRLVGESRRPVLYVGGGCAASGEELRRFVELTGIPVTTTL
MGLGNFPSDDPLSLRMLGMHGTVYANYAVDKADLLLALGVRFDDRVTGKI
EAFASRAKIVHVDIDPAEIGKNKQPHVSICADVKLALQGMNALLEGSTSK
KSFDFGSWNDELDQQKREFPLGYKTSNEEIQPQYAIQVLDELTKGEAIIG
TGVGQHQMWAAQYYTYKRPRQWLSSAGLGAMGFGLPAAAGASVANPGVTV
VDIDGDGSFLMNVQELAMIRIENLPVKVFVLNNQHLGMVVQWEDRFYKAN
RAHTYLGNPENESEIYPDFVTIAKGFNIPAVRVTKKNEVRAAIKKMLETP
GPYLLDIIVPHQEHVLPMIPSGGAFKDMILDGDGRTVY

An hra-like mutated version of ALS1 protein sequence:

SEQ ID NO: 38 provides a design for a variant Zm00001eb241810_P001 sequence with hra-like
mutations towards herbicide tolerance, wherein Zm00001eb241810_P001 is an acetolactate
synthase 1 protein of Zea mays
MATAAAASTALTGATTAAPKARRRAHLLATRRALAAPIRCSAASPAMPMAPPATPLRPWGPTDPRKGADILVESLERCGVRDV

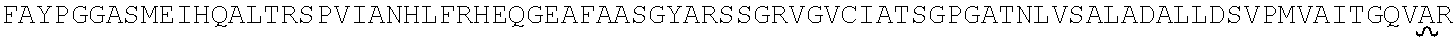

RMIGTDAFQETPIVEVTRSITKHNYLVLDVDDIPRVVQEAFFLASSGRPGPVLVDIPKDIQQQMAVPVWDKPMSLPGYIARLP
KPPATELLEQVLRLVGESRRPVLYVGGGCAASGEELRRFVELTGIPVTTTLMGLGNFPSDDPLSLRMLGMHGTVYANYAVDKA
DLLLALGVRFDDRVTGKIEAFASRAKIVHVDIDPAEIGKNKQPHVSICADVKLALQGMNALLEGSTSKKSFDFGSWNDELDQQ
KREFPLGYKTSNEEIQPQYAIQVLDELTKGEAIIGTGVGQHQMWAAQYYTYKRPRQWLSSAGLGAMGFGLPAAAGASVANPGV

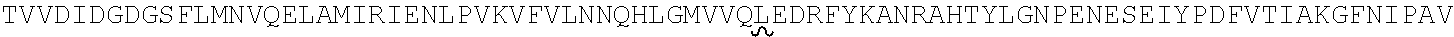

RVTKKNEVRAAIKKMLETPGPYLLDIIVPHQEHVLPMIPSGGAFKDMILDGDGRTVY

Example Non-Tolerant Reference ALS1 CDS RNA Sequence (Reference Level, −MFE=756; [0-1] design level=40.5%):

SEQ ID NO: 39 provides the nucleic acid sequence of the Zm00001eb241810_T001
CDS encoding of the Zm00001eb241810_P001 variant of a herbicide-susceptible
acetolactate synthase (ALS1) protein from Zea mays

AUGGCCACCGCCGCCGCCGCGUCUACCGCGCUCACUGGCGCCACUACCGCUGCGCCCAAGGCGAGGCGCCGGGCGCACCUCCU
GGCCACCCGCCGCGCCCUCGCCGCGCCCAUCAGGUGCUCAGCGGCGUCACCCGCCAUGCCGAUGGCUCCCCCGGCCACCCCGC
UCCGGCCGUGGGGCCCCACCGAUCCCCGCAAGGGCGCCGACAUCCUCGUCGAGUCCCUCGAGCGCUGCGGCGUCCGCGACGUC
UUCGCCUACCCCGGCGGCGCGUCCAUGGAGAUCCACCAGGCACUCACCCGCUCCCCCGUCAUCGCCAACCACCUCUUCCGCCA
CGAGCAAGGGGAGGCCUUUGCGGCCUCCGGCUACGCGCGCUCCUCGGGCCGCGUCGGCGUCUGCAUCGCCACCUCCGGCCCCG
GCGCCACCAACCUUGUCUCCGCGCUCGCCGACGCGCUGCUCGAUUCCGUCCCCAUGGUCGCCAUCACGGGACAGGUGCCGCGA
CGCAUGAUUGGCACCGACGCCUUCCAGGAGACGCCCAUCGUCGAGGUCACCCGCUCCAUCACCAAGCACAACUACCUGGUCCU
CGACGUCGACGACAUCCCCCGCGUCGUGCAGGAGGCUUUCUUCCUCGCCUCCUCUGGUCGACCGGGGCCGGUGCUUGUCGACA
UCCCCAAGGACAUCCAGCAGCAGAUGGCGGUGCCUGUCUGGGACAAGCCCAUGAGUCUGCCUGGGUACAUUGCGCGCCUUCCC
AAGCCCCCUGCGACUGAGUUGCUUGAGCAGGUGCUGCGUCUUGUUGGUGAAUCCCGGCGCCCUGUUCUUUAUGUUGGCGGUGG
CUGCGCAGCAUCUGGUGAGGAGUUGCGACGCUUUGUGGAGCUGACUGGAAUCCCGGUCACAACUACUCUUAUGGGCCUCGGCA
ACUUCCCCAGCGACGACCCACUGUCUCUGCGCAUGCUAGGUAUGCAUGGCACGGUGUAUGCAAAUUAUGCAGUGGAUAAGGCC
GAUCUGUUGCUUGCACUUGGUGUGCGGUUUGAUGAUCGUGUGACAGGGAAGAUUGAGGCUUUUGCAAGCAGGGCUAAGAUUGU
GCACGUUGAUAUUGAUCCGGCUGAGAUUGGCAAGAACAAGCAGCCACAUGUGUCCAUCUGUGCAGAUGUUAAGCUUGCUUUGC
AGGGCAUGAAUGCUCUUCUUGAAGGAAGCACAUCAAAGAAGAGCUUUGACUUUGGCUCAUGGAACGAUGAGUUGGAUCAGCAG
AAGAGGGAAUUCCCCCUUGGGUAUAAAACAUCUAAUGAGGAGAUCCAGCCACAAUAUGCUAUUCAGGUUCUUGAUGAGCUGAC
GAAAGGCGAGGCCAUCAUCGGCACAGGUGUUGGGCAGCACCAGAUGUGGGCGGCACAGUACUACACUUACAAGCGGCCAAGGC
AGUGGUUGUCUUCAGCUGGUCUUGGGGCUAUGGGAUUUGGUUUGCCGGCUGCUGCUGGUGCUUCUGUGGCCAACCCAGGUGUU
ACUGUUGUUGACAUCGAUGGAGAUGGUAGCUUUCUCAUGAACGUUCAGGAGCUAGCUAUGAUCCGAAUUGAGAACCUCCCGGU
GAAGGUCUUUGUGCUAAACAACCAGCACCUGGGGAUGGUGGUGCAGUGGGAGGACAGGUUCUAUAAGGCCAACAGAGCGCACA
CAUACUUGGGAAACCCAGAGAAUGAAAGUGAGAUAUAUCCAGAUUUCGUGACGAUCGCCAAAGGGUUCAACAUUCCAGCGGUC
CGUGUGACAAAGAAGAACGAAGUCCGCGCAGCGAUAAAGAAGAUGCUCGAGACUCCAGGGCCGUACCUCUUGGAUAUAAUCGU
CCCACACCAGGAGCAUGUGUUGCCUAUGAUCCCUAGUGGUGGGGCUUUCAAGGAUAUGAUCCUGGAUGGUGAUGGCAGGACUG
UGUACUGA

hra-like mutated ALS1 CDS RNA w/Selected Differences from reference underlined (−MFE=813, [0-1] design level=45.2%):

SEQ ID NO: 40 provides a design for a nucleic acid sequence with CDS trained to
a 45.2% expression design level encoding of a synthetic design of a variant of
Zm00001eb241810_P001 with hra-like mutations towards herbicide tolerance,
wherein Zm00001eb241810_P001 is an acetolactate synthase 1 protein of Zea mays

AUGGCCACGGCCGCCACGGCCGCAGCGGCGUUGACGGGCGCGACGACCGCGACGCCAAAGUCGCGGCGUCGCGCCCAUCACCU
GGCGACACGCCGGGCGCUGGCGGCGCCGAUUCGGUGUAGCGCGCUAUCGCGCGCUACACCGACGGCGCCCCCGGCCACCCCGC
UCCGGCCGUGGGGCCCCACCGAUCCCCGCAAGGGCGCCGACAUCCUCGUCGAGUCCCUCGAGCGCUGCGGCGUCCGCGACGUC
UUCGCCUACCCCGGCGGCGCGUCCAUGGAGAUCCACCAGGCACUCACCCGCUCCCCCGUCAUCGCCAACCACCUCUUCCGCCA
CGAGCAAGGGGAGGCCUUUGCGGCCUCCGGCUACGCGCGCUCCUCGGGCCGCGUCGGCGUCUGCAUCGCCACCUCCGGCCCCG
GCGCCACCAACCUUGUCUCCGCGCUCGCCGACGCGCUGCUCGAUUCCGUCCCCAUGGUCGCCAUCACGGGACAGGUGGCGCGA
CGCAUGAUUGGCACCGACGCCUUCCAGGAGACGCCCAUCGUCGAGGUCACCCGCUCCAUCACCAAGCACAACUACCUGGUCCU
CGACGUCGACGACAUCCCCCGCGUCGUGCAGGAGGCUUUCUUCCUCGCCUCCUCUGGUCGACCGGGGCCGGUGCUUGUCGACA
UCCCCAAGGACAUCCAGCAGCAGAUGGCGGUGCCUGUCUGGGACAAGCCCAUGAGUCUGCCUGGGUACAUUGCGCGCCUUCCC
AAGCCCCCUGCGACUGAGUUGCUUGAGCAGGUGCUGCGUCUUGUUGGUGAAUCCCGGCGCCCUGUUCUUUAUGUUGGCGGUGG
CUGCGCAGCAUCUGGUGAGGAGUUGCGACGCUUUGUGGAGCUGACUGGAAUCCCGGUCACAACUACUCUUAUGGGCCUCGGCA
ACUUCCCCAGCGACGACCCACUGUCUCUGCGCAUGCUAGGUAUGCAUGGCACGGUGUAUGCAAAUUAUGCAGUGGAUAAGGCC
GAUCUGUUGCUUGCACUUGGUGUGCGGUUUGAUGAUCGUGUGACAGGGAAGAUUGAGGCUUUUGCAAGCAGGGCUAAGAUUGU
GCACGUUGAUAUUGAUCCGGCUGAGAUUGGCAAGAACAAGCAGCCACAUGUGUCCAUCUGUGCAGAUGUUAAGCUUGCUUUGC
AGGGCAUGAAUGCUCUUCUUGAAGGAAGCACAUCAAAGAAGAGCUUUGACUUUGGCUCAUGGAACGAUGAGUUGGAUCAGCAG
AAGAGGGAAUUCCCCCUUGGGUAUAAAACAUCUAAUGAGGAGAUCCAGCCACAAUAUGCUAUUCAGGUUCUUGAUGAGCUGAC
GAAAGGCGAGGCCAUCAUCGGCACAGGUGUUGGGCAGCACCAGAUGUGGGCGGCACAGUACUACACUUACAAGCGGCCAAGGC
AGUGGUUGUCUUCAGCUGGUCUUGGGGCUAUGGGAUUUGGUUUGCCGGCUGCUGCUGGUGCUUCUGUGGCCAACCCAGGUGUU
ACUGUUGUUGACAUCGAUGGAGAUGGUAGCUUUCUCAUGAACGUUCAGGAGCUAGCUAUGAUCCGAAUUGAGAACCUCCCGGU
GAAGGUCUUUGUGCUAAACAACCAGCACCUGGGGAUGGUGGUGCAGCUGGAGGACAGGUUCUAUAAGGCCAACAGAGCGCACA
CAUACUUGGGAAACCCAGAGAAUGAAAGUGAGAUAUAUCCAGAUUUCGUGACGAUCGCCAAAGGGUUCAACAUUCCAGCGGUC
CGUGUGACAAAGAAGAACGAAGUCCGCGCAGCGAUAAAGAAGAUGCUCGAGACUCCAGGGCCGUACCUCUUGGAUAUAAUCGU
CCCACACCAGGAGCAUGUGUUGCCUAUGAUCCCUAGUGGUGGGGCUUUCAAGGAUAUGAUCCUGGAUGGUGAUGGCAGGACUG
UGUACUGA

Example Trained hra-like mutated ALS1 Coding Strand DNA Excerpt w/coding regions underlined and example differences from reference highlighted in bold with wavy underline (Template Strand putatively transcribed to primary transcript):

SEQ ID NO: 41 provides a design for a polynucleotide sequence to help identify what needs
DNA editing with CDS trained to a 45.2% expression design level with putative encoding of a
design for a variant Zm00001eb241810_P001 sequence with hra-like mutations, wherein
Zm00001eb241810_P001 is an acetolactate synthase 1 protein of Zea mays
CCATTCCCATCACCATCTGAGCCACACATCCTCTGAACAAAAGCAGGGAGGCCTCTACGCACATCCCCCTTTCTCCCACTCCG

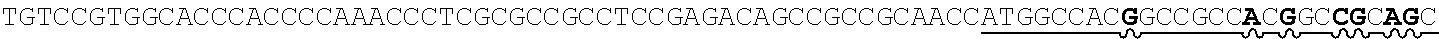

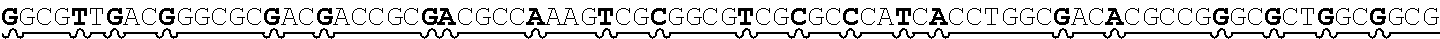

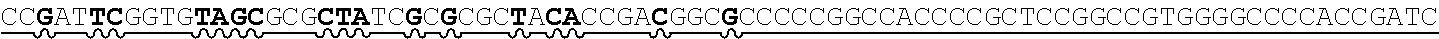

CCCGCAAGGGCGCCGACATCCTCGTCGAGTCCCTCGAGCGCTGCGGCGTCCGCGACGTCTTCGCCTACCCCGGCGGCGCGTCC
ATGGAGATCCACCAGGCACTCACCCGCTCCCCCGTCATCGCCAACCACCTCTTCCGCCACGAGCAAGGGGAGGCCTTTGCGGC
CTCCGGCTACGCGCGCTCCTCGGGCCGCGTCGGCGTCTGCATCGCCACCTCCGGCCCCGGCGCCACCAACCTTGTCTCCGCGC

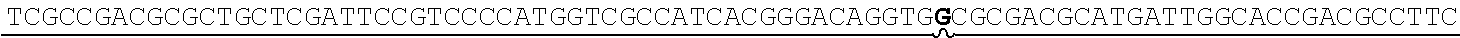

CAGGAGACGCCCATCGTCGAGGTCACCCGCTCCATCACCAAGCACAACTACCTGGTCCTCGACGTCGACGACATCCCCCGCGT
CGTGCAGGAGGCTTTCTTCCTCGCCTCCTCTGGTCGACCGGGGCCGGTGCTTGTCGACATCCCCAAGGACATCCAGCAGCAGA
TGGCGGTGCCTGTCTGGGACAAGCCCATGAGTCTGCCTGGGTACATTGCGCGCCTTCCCAAGCCCCCTGCGACTGAGTTGCTT
GAGCAGGTGCTGCGTCTTGTTGGTGAATCCCGGCGCCCTGTTCTTTATGTTGGCGGTGGCTGCGCAGCATCTGGTGAGGAGTT
GCGACGCTTTGTGGAGCTGACTGGAATCCCGGTCACAACTACTCTTATGGGCCTCGGCAACTTCCCCAGCGACGACCCACTGT
CTCTGCGCATGCTAGGTATGCATGGCACGGTGTATGCAAATTATGCAGTGGATAAGGCCGATCTGTTGCTTGCACTTGGTGTG
CGGTTTGATGATCGTGTGACAGGGAAGATTGAGGCTTTTGCAAGCAGGGCTAAGATTGTGCACGTTGATATTGATCCGGCTGA
GATTGGCAAGAACAAGCAGCCACATGTGTCCATCTGTGCAGATGTTAAGCTTGCTTTGCAGGGCATGAATGCTCTTCTTGAAG
GAAGCACATCAAAGAAGAGCTTTGACTTTGGCTCATGGAACGATGAGTTGGATCAGCAGAAGAGGGAATTCCCCCTTGGGTAT
AAAACATCTAATGAGGAGATCCAGCCACAATATGCTATTCAGGTTCTTGATGAGCTGACGAAAGGCGAGGCCATCATCGGCAC
AGGTGTTGGGCAGCACCAGATGTGGGCGGCACAGTACTACACTTACAAGCGGCCAAGGCAGTGGTTGTCTTCAGCTGGTCTTG
GGGCTATGGGATTTGGTTTGCCGGCTGCTGCTGGTGCTTCTGTGGCCAACCCAGGTGTTACTGTTGTTGACATCGATGGAGAT
GGTAGCTTTCTCATGAACGTTCAGGAGCTAGCTATGATCCGAATTGAGAACCTCCCGGTGAAGGTCTTTGTGCTAAACAACCA

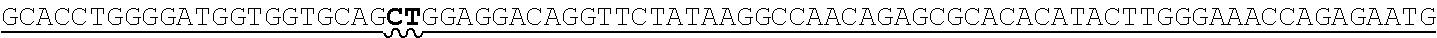

AAAGTGAGATATATCCAGATTTCGTGACGATCGCCAAAGGGTTCAACATTCCAGCGGTCCGTGTGACAAAGAAGAACGAAGTC
CGCGCAGCGATAAAGAAGATGCTCGAGACTCCAGGGCCGTACCTCTTGGATATAATCGTCCCACACCAGGAGCATGTGTTGCC
TATGATCCCTAGTGGTGGGGCTTTCAAGGATATGATCCTGGATGGTGATGGCAGGACTGTGTACTGATCTAAAATCCAGCAAG
CAACTGATCTAAAATCCAGCAAGCACCGCCTCCCTGCTAGTACAAGGGTGATATGTTTTTATCTGTGTGATGTTCTCCTGTAT
TCTATCTTTTTTTGTAGGCCGTCAGCTATCTGTTATGGTAATCCTATGTAGCTTCCGACCTTGTAATTGTGTAGTCTGTTGTT
TTCCTTCTGGCATGTGTCATAAGAGATCATTTAAGTGCCTTTTGCTACATATAAATAAGATAATAAGCACTGCTATGCAGTGG
TTCTGAATTGGCTTCTGTTGCCAAATTTAAGTGTCCAACTGGTCCTTGCTTTTGTTTTCGCTATTTTTTTCCTTTTTTAGTTA
TTATTATATTGGTAATTTCAACTCAACATATGATGTATGGAATAATGCTAGGGCTGCAATTTCAAACTATTTTACAAACCAGA
ATGGCATTTTCGTGGTTTGA

Zm00001eb180890_P₀₀₁ is a non-herbicide tolerant ALS2 protein sequence:

SEQ ID NO: 42 provides the amino acid sequence
of the Zm00001eb180890_P001 variant of an
herbicide-susceptible acetolactate synthase
2 protein of Zea mays

MATAATAAAALTGATTATPKSRRRAHHLATRRALAAPIRCSALSRATPTA
PPATPLRPWGPNEPRKGSDILVEALERCGVRDVFAYPGGASMEIHQALTR
SPVIANHLFRHEQGEAFAASGYARSSGRVGVCIATSGPGATNLVSALADA
LLDSVPIVAITGQVPRRMIGTDAFQETPIVEVTRSITKHNYLVLDVDDIP
RVVQEAFFLASSGRPGPVLVDIPKDIQQQMAVPAWDTPMSLPGYIARLPK
PPATEFLEQVLRLVGESRRPVLYVGGGCAASGEELCRFVELTGIPVTTTL
MGLGNFPSDDPLSLRMLGMHGTVYANYAVDKADLLLAFGVRFDDRVTGKI
EAFAGRAKIVHIDIDPAEIGKNKQPHVSICADVKLALQGMNTLLEGSTSK
KSFDFGSWHDELDQQKREFPLGYKIFNEEIQPQYAIQVLDELTKGKAIIA
TGVGQHQMWAAQYYTYKRPRQWLSSAGLGAMGFGLPAAAGAAVANPGVTV
VDIDGDGSFLMNIQELAMIRIENLPVKVFVLNNQHLGMVVQWEDRFYKAN
RAHTFLGNPENESEIYPDFVAIAKGFNIPAVRVTKKSEVHAAIKKMLEAP
GPYLLDIIVPHQEHVLPMIPSGGAFKDMILDGDGRTVY

An herbicide tolerant version of ALS2 protein sequence:

SEQ ID NO: 43 provides a design for a variant Zm00001eb180890_P001 sequence with hra-like
mutations towards herbicide tolerance, wherein Zm00001eb180890_P001 is an acetolactate
synthase 2 protein of Zea mays
MATAATAAAALTGATTATPKSRRRAHHLATRRALAAPIRCSALSRATPTAPPATPLRPWGPNEPRKGSDILVEALERCGVRDV

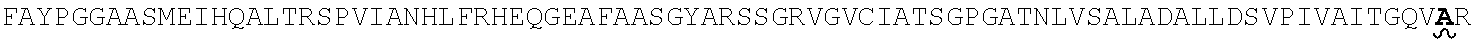

RMIGTDAFQETPIVEVTRSITKHNYLVLDVDDIPRVVQEAFFLASSGRPGPVLVDIPKDIQQQMAVPAWDTPMSLPGYIARLP
KPPATEFLEQVLRLVGESRRPVLYVGGGCAASGEELCRFVELTGIPVTTTLMGLGNFPSDDPLSLRMLGMHGTVYANYAVDKA
DLLLAFGVRFDDRVTGKIEAFAGRAKIVHIDIDPAEIGKNKQPHVSICADVKLALQGMNTLLEGSTSKKSFDFGSWHDELDQQ
KREFPLGYKIFNEEIQPQYAIQVLDELTKGKAIIATGVGQHQMWAAQYYTYKRPRQWLSSAGLGAMGFGLPAAAGAAVANPGV

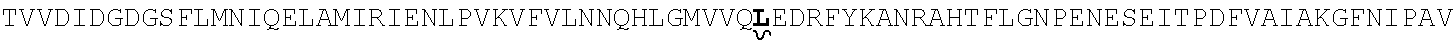

RVTKKSEVHAAIKKMLEAPGPYLLDIIVPHQEHVLPMIPSGGAFKDMILDGDGRTVY

Example Non-Tolerant Reference ALS2 CDS RNA Sequence (Reference Level, −MFE=736; [0-1] design level=38.9%):

SEQ ID NO: 44 provides the nucleic acid sequence of the Zm00001eb180890_T001 CDS
encoding of the Zm00001eb180890_P001 variant of a different herbicide-susceptible
acetolactate synthase (ALS2) protein from Zea mays

AUGGCCACCGCCGCCACCGCGGCCGCCGCGCUCACCGGCGCCACUACOGCUACGCCCAAGUCGAGGCGCCGAGCCCACCACUU
GGCCACCCGGCGCGCCCUCGCCGCGCCCAUCAGGUGCUCAGCGUUGUCACGCGCCACGCCGACGGCUCCCCCGGCCACUCCGC
UACGUCCGUGGGGCCCCAACGAGCCCCGCAAGGGCUCCGACAUCCUCGUCGAGGCUCUCGAGCGCUGUGGCGUCCGUGACGUC
UUCGCCUACCCCGGCGGCGCAUCCAUGGAGAUCCACCAGGCACUCACCCGCUCCCCCGUCAUCGCCAACCACCUCUUCCGCCA
CGAACAAGGGGAGGCCUUCGCCGCCUCCGGCUACGCGCGCUCCUCGGGCCGCGUUGGCGUCUGCAUCGCCACCUCCGGCCCCG
GCGCCACCAACCUAGUCUCUGCGCUCGCAGACGCGUUGCUCGACUCCGUCCCCAUUGUCGCCAUCACGGGACAGGUGGCGCGA
CGCAUGAUUGGCACCGACGCCUUUCAGGAGACGCCCAUCGUCGAGGUCACCCGCUCCAUCACCAAGCACAACUACCUGGUCCU
CGACGUCGACGACAUCCCCCGCGUCGUGCAGGAGGCCUUCUUCCUCGCAUCCUCUGGUCGCCCGGGGCCGGUGCUUGUUGACA
UCCCCAAGGACAUCCAGCAGCAGAUGGCGGUGCCGGCCUGGGACACGCCCAUGAGUCUGCCUGGGUACAUCGCGCGCCUUCCC
AAGCCUCCCGCGACUGAAUUUCUUGAGCAGGUGCUGCGUCUUGUUGGUGAAUCACGGCGCCCUGUUCUUUAUGUUGGCGGUGG
CUGUGCAGCAUCAGGUGAGGAGUUGUGCCGCUUUGUGGAGUUGACUGGAAUCCCAGUCACAACUACUCUUAUGGGCCUUGGCA
ACUUCCCCAGCGACGACCCACUGUCACUGCGCAUGCUUGGUAUGCAUGGCCAGUGUAUGCAAAUUAUGCAGUGGAUAAGGCCG
AUCUGUUGCUUGCAUUUGGUGUGCGGUUUGAUGAUCGUGUGACAGGGAAAAUUGAGGCUUUUGCAGGCAGAGCUAAGAUUGUG
CACAUUGAUAUUGAUCCUGCUGAGAUUGGCAAGAACAAGCAGCCACAUGUGUCCAUCUGUGCAGAUGUUAAGCUUGCUUUGCA
GGGCAUGAAUACUCUUCUGGAAGGAAGCACAUCAAAGAAGAGCUUUGACUUCGGCUCAUGGCAUGAUGAAUUGGAUCAGCAAA
AGCGGGAGUUUCCCCUUGGGUAUAAAAUCUUCAAUGAGGAAAUCCAGCCACAAUAUGCUAUUCAGGUUCUUGAUGAGUUGACG
AAGGGGAAGGCCAUCAUUGCCACAGGUGUUGGGCAGCACCAGAUGUGGGCGGCACAGUAUUACACUUACAAGCGGCCAAGGCA
GUGGCUGUCUUCAGCUGGUCUUGGGGCUAUGGGAUUUGGUUUGCCGGCUGCUGCUGGUGCUGCUGUGGCCAACCCAGGUGUCA
CUGUUGUUGACAUCGACGGAGAUGGUAGCUUCCUCAUGAACAUUCAGGAGCUAGCUAUGAUCCGUAUUGAGAACCUCCCAGUC
AAGGUCUUUGUGCUAAACAACCAGCACCUCGGGAUGGUGGUGCAGCUGGAGGACAGGUUCUAUAAGGCCAAUAGAGCACACAC
AUUCUUGGGAAACCCAGAGAACGAAAGUGAGAUAUAUCCAGAUUUUGUGGCAAUUGCCAAAGGGUUCAACAUUCCAGCAGUCC
GUGUGACAAAGAAGAGCGAAGUCCAUGCAGCAAUCAAGAAGAUGCUUGAGGCUCCAGGGCCGUACCUCUUGGAUAUAAUCGUC
CCGCACCAGGAGCAUGUGUUGCCUAUGAUCCCUAGUGGUGGGGCUUUCAAGGAUAUGAUCCUGGAUGGUGAUGGCAGGACUGU
GUAUUGA

Designed Tolerant ALS2 CDS w/the example selected differences from reference underlined (−MFE=812, [0-1] design level=45.2%):

SEQ ID NO: 45 provides a design for a nucleic acid sequence with CDS
trained to a 45.2% expression design level encoding of a design for a
Zm00001eb180890_P001 variant with hra-like mutations, wherein
Zm00001eb180890_P001 is an acetolactate synthase 2 protein of Zea mays

AUGGCCACGGCCGCCACGGCCGCAGCGGCGUUGACGGGCGCGACGACCGCGACGCCAAAGUCGCGGCGUCGCGCCCAUCACCU
GGCGACACGCCGGGCGCUGGCGGCGCCGAUUCGGUGUAGCGCGCUAUCGCGCGCUACACCGACGGCGCCGCCAGCGACGCCGC
UACGUCCGUGGGGCCCCAACGAGCCCCGCAAGGGCUCCGACAUCCUCGUCGAGGCUCUCGAGCGCUGUGGCGUCCGUGACGUC
UUCGCCUACCCCGGCGGCGCAUCCAUGGAGAUCCACCAGGCACUCACCCGCUCCCCCGUCAUCGCCAACCACCUCUUCCGCCA
CGAACAAGGGGAGGCCUUCGCCGCCUCCGGCUACGCGCGCUCCUCGGGCCGCGUUGGCGUCUGCAUCGCCACCUCCGGCCCCG
GCGCCACCAACCUAGUCUCUGCGCUCGCAGACGCGUUGCUCGACUCCGUCCCCAUUGUCGCCAUCACGGGACAGGUGGCGCGA
CGCAUGAUUGGCACCGACGCCUUUCAGGAGACGCCCAUCGUCGAGGUCACCCGCUCCAUCACCAAGCACAACUACCUGGUCCU
CGACGUCGACGACAUCCCCCGCGUCGUGCAGGAGGCCUUCUUCCUCGCAUCCUCUGGUCGCCCGGGGCCGGUGCUUGUUGACA
UCCCCAAGGACAUCCAGCAGCAGAUGGCGGUGCCGGCCUGGGACACGCCCAUGAGUCUGCCUGGGUACAUCGCGCGCCUUCCC
AAGCCUCCCGCGACUGAAUUUCUUGAGCAGGUGCUGCGUCUUGUUGGUGAAUCACGGCGCCCUGUUCUUUAUGUUGGCGGUGG
CUGUGCAGCAUCAGGUGAGGAGUUGUGCCGCUUUGUGGAGUUGACUGGAAUCCCAGUCACAACUACUCUUAUGGGCCUUGGCA
ACUUCCCCAGCGACGACCCACUGUCACUGCGCAUGCUUGGUAUGCAUGGCACAGUGUAUGCAAAUUAUGCAGUGGAUAAGGCC
GAUCUGUUGCUUGCAUUUGGUGUGCGGUUUGAUGAUCGUGUGACAGGGAAAAUUGAGGCUUUUGCAGGCAGAGCUAAGAUUGU
GCACAUUGAUAUUGAUCCUGCUGAGAUUGGCAAGAACAAGCAGCCACAUGUGUCCAUCUGUGCAGAUGUUAAGCUUGCUUUGC
AGGGCAUGAAUACUCUUCUGGAAGGAAGCACAUCAAAGAAGAGCUUUGACUUCGGCUCAUGGCAUGAUGAAUUGGAUCAGCAA
AAGCGGGAGUUUCCCCUUGGGUAUAAAAUCUUCAAUGAGGAAAUCCAGCCACAAUAUGCUAUUCAGGUUCUUGAUGAGUUGAC
GAAGGGGAAGGCCAUCAUUGCCACAGGUGUUGGGCAGCACCAGAUGUGGGCGGCACAGUAUUACACUUACAAGCGGCCAAGGC
AGUGGCUGUCUUCAGCUGGUCUUGGGGCUAUGGGAUUUGGUUUGCCGGCUGCUGCUGGUGCUGCUGUGGCCAACCCAGGUGUC
ACUGUUGUUGACAUCGACGGAGAUGGUAGCUUCCUCAUGAACAUUCAGGAGCUAGCUAUGAUCCGUAUUGAGAACCUCCCAGU
CAAGGUCUUUGUGCUAAACAACCAGCACCUCGGGAUGGUGGUGCAGCUGGAGGACAGGUUCUAUAAGGCCAAUAGAGCACACA
CAUUCUUGGGAAACCCAGAGAACGAAAGUGAGAUAUAUCCAGAUUUUGUGGCAAUUGCCAAAGGGUUCAACAUUCCAGCAGUC
CGUGUGACAAAGAAGAGCGAAGUCCAUGCAGCAAUCAAGAAGAUGCUUGAGGCUCCAGGGCCGUACCUCUUGGAUAUAAUCGU
CCCGCACCAGGAGCAUGUGUUGCCUAUGAUCCCUAGUGGUGGGGCUUUCAAGGAUAUGAUCCUGGAUGGUGAUGGCAGGACUG
UGUAUUGA

Example Trained ALS2 Coding Strand DNA Excerpt w/differences from reference highlighted in bold with wavy underline (Template Strand putatively transcribed to primary transcript):

SEQ ID NO: 46 provides a design for a polynucleotide sequence to help identify what needs
DNA editing with CDS trained to a 45.2% expression design level with putative encoding of a
design of a Zm00001eb180890_P001 variant with hra-like mutations, wherein
Zm00001eb180890_P001 is an acetolactate synthase 2 protein of Zea mays
CCCCCTTTCCCACAATCCCACTCCGTGCCAGGTGCCACCCTCCCCAAGCCCTCGCGCCGCCTCCGAGACAGCCGCCCGCAACC

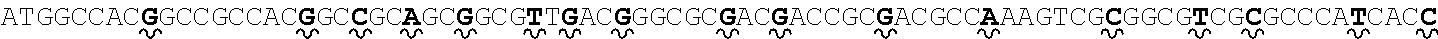

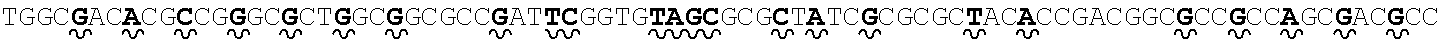

GCTACGTCCGTGGGGCCCCAACGAGCCCCGCAAGGGCTCCGACATCCTCGTCGAGGCTCTCGAGCGCTGTGGCGTCCGTGACG
TCTTCGCCTACCCCGGCGGCGCATCCATGGAGATCCACCAGGCACTCACCCGCTCCCCCGTCATCGCCAACCACCTCTTCCGC
CACGAACAAGGGGAGGCCTTCGCCGCCTCCGGCTACGCGCGCTCCTCGGGCCGCGTTGGCGTCTGCATCGCCACCTCCGGCCC

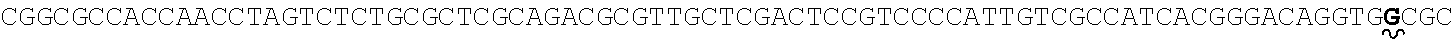

GACGCATGATTGGCACCGACGCCTTTCAGGAGACGCCCATCGTCGAGGTCACCCGCTCCATCACCAAGCACAACTACCTGGTC
CTCGACGTCGACGACATCCCCCGCGTCGTGCAGGAGGCCTTCTTCCTCGCATCCTCTGGTCGCCCGGGGCCGGTGCTTGTTGA
CATCCCCAAGGACATCCAGCAGCAGATGGCGGTGCCGGCCTGGGACACGCCCATGAGTCTGCCTGGGTACATCGCGCGCCTTC
CCAAGCCTCCCGCGACTGAATTTCTTGAGCAGGTGCTGCGTCTTGTTGGTGAATCACGGCGCCCTGTTCTTTATGTTGGCGGT
GGCTGTGCAGCATCAGGTGAGGAGTTGTGCCGCTTTGTGGAGTTGACTGGAATCCCAGTCACAACTACTCTTATGGGCCTTGG
CAACTTCCCCAGCGACGACCCACTGTCACTGCGCATGCTTGGTATGCATGGCACAGTGTATGCAAATTATGCAGTGGATAAGG
CCGATCTGTTGCTTGCATTTGGTGTGCGGTTTGATGATCGTGTGACAGGGAAAATTGAGGCTTTTGCAGGCAGAGCTAAGATT
GTGCACATTGATATTGATCCTGCTGAGATTGGCAAGAACAAGCAGCCACATGTGTCCATCTGTGCAGATGTTAAGCTTGCTTT
GCAGGGCATGAATACTCTTCTGGAAGGAAGCACATCAAAGAAGAGCTTTGACTTCGGCTCATGGCATGATGAATTGGATCAGC
AAAAGCGGGAGTTTCCCCTTGGGTATAAAATCTTCAATGAGGAAATCCAGCCACAATATGCTATTCAGGTTCTTGATGAGTTG
ACGAAGGGGAAGGCCATCATTGCCACAGGTGTTGGGCAGCACCAGATGTGGGCGGCACAGTATTACACTTACAAGCGGCCAAG
GCAGTGGCTGTCTTCAGCTGGTCTTGGGGCTATGGGATTTGGTTTGCCGGCTGCTGCTGGTGCTGCTGTGGCCAACCCAGGTG
TCACTGTTGTTGACATCGACGGAGATGGTAGCTTCCTCATGAACATTCAGGAGCTAGCTATGATCCGTATTGAGAACCTCCCA

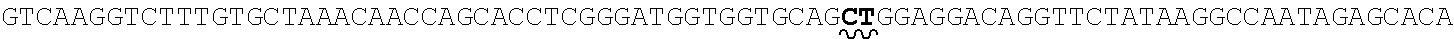

CACATTCTTGGGAAACCCAGAGAACGAAAGTGAGATATATCCAGATTTTGTGGCAATTGCCAAAGGGTTCAACATTCCAGCAG
TCCGTGTGACAAAGAAGAGCGAAGTCCATGCAGCAATCAAGAAGATGCTTGAGGCTCCAGGGCCGTACCTCTTGGATATAATC
GTCCCGCACCAGGAGCATGTGTTGCCTATGATCCCTAGTGGTGGGGCTTTCAAGGATATGATCCTGGATGGTGATGGCAGGAC
TGTGTATTGATCTAAAGTTCAGCAAGCACTGCCTACCTGCCTATCTTTGACATGCATGAGCTAGTACAAGTGTGATATGTTTT
TATCAATGTGATGGTACTCTGTTATGGTAATCTTAAGTAGCATCCAACCCTGTGTGTAGTATGTTGTTTTCGTGTTGGCATAT
GTTTCAGAAGCCATCATGTAAGTGCCTTTTACTACATATAAATAAGGTAATAAGCATTGTTATGCACCGGTTCTGAATTGGTC
TTCTTTTGCCAAATATAGGTCCTGTTTGATACCTATAGCTCTAGAAAATTTGGTGTGGTTGGTGGAGCAGGCCATTAGGTGTT
CCATAAAATAGTGGAGTTGTAAGACTTTAGAAGACATTTAGATAAATTATTTTATTTATTATTTA

### H. Example 8: Experimental Gene Training towards Greater Development

In this example, we focus on precisely redesigning expression of an existing gene in a maize inbred toward allowing for increased hormone expression as a step towards greater floral-trait-linked development, better productivity, and higher yields-instead of shorter stature when damaging wind events are not expected. For example, the splice of GA20ox3 In this example, the Zm00001eb366090_T001 splice sequence of GA20ox3 (i.e. gibberellin 20-oxidase 3 aka gibberellin 20-oxidase 5) from the B73 reference is trained to a higher relative expression design level under UTR sequence and structure constraints.

Zm00001eb366090_P₀₀₁ is a GA20ox3 protein sequence:

SEQ ID NO: 47 provides the amino acid sequence
of the Zm00001eb366090_P001 variant of an
GA200x3 protein of Zea mays

MRPRLPPNVPSLPSSLSLLANSLSSPVTNTPTRPDSFPAYLQLAHLMVSQ
ERQEPAVPSSSSSSAKRAATSMDASPAPPLLLRAPTPSPSIDLPAGKDKA
DAAASKAGAAVFDLRREPKIPAPFLWPQEEARPSSAAELEVPMVDVGVLR
NGDRAGLRRAAAQVAAACATHGFFQVCGHGVDAALGRAALDGASDFFRLP
LAEKQRARRVPGTVSGYTSAHADRFAAKLPWKETLSFGYHDGAASPVVVD
YFVGTLGQDFEPMGWVYQRYCEEMKELSLTIMELLELSLGVELRGYYREF
FEDSRSIMRCNYYPPCPEPERTLGTGPHCDPTALTILLQDDVGGLEVLVD
GEWRPVRPVPGAMVINIGDTFMALSNGRYKSCLHRAVVNQRRARRSLAFF
LCPREDRVVRPPASAAPRRYPDFTWADLMRFTQRHYRADTRTLDAFTRWL
SHGPAQAAAPPCT

Example Reference GA20ox3 CDS RNA Sequence (Reference Level, −MFE=706, corresponding mRNA half-life=0.61 hours estimated; [0-1] design level=53.4%=(706-203)/(1145-203)

SEQ ID NO: 48 provides the nucleic acid sequence of the Zm00001eb366090_T001 CDS
encoding of the Zm00001eb366090_P001 variant of a GA200x3 protein from Zea mays

AUGAGGCCGCGCCUCCCUCCAAAUGUUCCCUCCCUGCCUUCGUCUUUGUCGUUGCUCGCAAACUCCCUGUCCUCCCCUGUUAC
AAAUACCCCCACCCGCCCGGACAGCUUCCCUGCAUACUUGCAGCUCGCACAUCUCAUGGUGUCGCAGGAACGACAAGAGCCAG
CUGUGCCUAGCAGCAGCAGCAGCAGCGCCAAGCGCGCAGCCACGUCCAUGGACGCCAGCCCGGCCCCGCCGCUCCUCCUCCGC
GCCCCCACUCCCAGCCCCAGCAUUGACCUCCCCGCUGGCAAGGACAAGGCCGACGCGGCGGCCAGCAAGGCCGGCGCGGCCGU
GUUCGACCUGCGCCGGGAGCCCAAGAUCCCCGCGCCAUUCCUGUGGCCGCAGGAAGAGGCGCGGCCGUCCUCGGCCGCGGAGC
UGGAGGUGCCGAUGGUGGACGUGGGCGUGCUGCGCAAUGGCGACCGCGCGGGGCUGCGGCGCGCCGCGGCGCAGGUGGCCGCG
GCGUGCGCGACGCACGGGUUCUUCCAGGUGUGCGGGCACGGCGUGGACGCGGCGCUGGGGCGCGCCGCGCUGGACGGCGCCAG
CGACUUCUUCCGGCUGCCGCUCGCCGAGAAGCAGCGCGCCCGGCGCGUCCCCGGCACCGUGUCCGGGUACACGAGCGCGCACG
CCGACCGGUUCGCGGCCAAGCUCCCCUGGAAGGAGACCCUGUCGUUCGGCUACCACGACGGCGCCGCGUCGCCUGUCGUCGUG
GACUACUUCGUCGGCACCCUCGGCCAGGAUUUCGAGCCAAUGGGGUGGGUGUACCAGAGGUACUGCGAGGAGAUGAAGGAGCU
GUCGCUGACGAUCAUGGAGCUGCUGGAGCUGAGCCUGGGCGUGGAGCUGCGCGGCUACUACCGGGAGUUCUUCGAGGACAGCC
GGUCCAUCAUGCGGUGCAACUACUACCCGCCGUGCCCGGAGCCGGAGCGCACGCUGGGCACGGGCCCGCACUGCGACCCCACG
GCGCUCACCAUCCUCCUGCAGGACGACGUGGGCGGGCUGGAGGUGCUGGUGGACGGUGAGUGGCGCCCCGUCCGGCCCGUCCC
GGGCGCCAUGGUCAUCAACAUCGGCGACACCUUCAUGGCGCUGUCGAACGGGAGGUACAAGAGCUGCCUGCACCGCGCGGUGG
UGAACCAGCGGGGGCGCGGCGGUCGCUGGCCUUCUUCCUGUGCCCGCGCGAGGACCGGGUGGUGCGCCCGCCGGCCAGUGCU
GCGCCGCGGCGCUACCCGGACUUCACCUGGGCCGACCUCAUGCGCUUCACGCAGCGCCACUACOGCGCCGACACCCGCACGCU
GGACGCCUUCACCCGCUGGCUCUCCCACGGCCCGGCCCAGGCGGCGGCGCCUCCCUGCACCUAG

Designed Example GA20ox3 CDS RNA w/selected differences underlined (−MFE=816, corresponding mRNA half-life=0.63 hours estimated, [0-1] design level=65.1%):

SEQ ID NO: 49 provides a design for a nucleic acid sequence with CDS trained to
a 65.1% expression design level encoding of a Zm00001eb366090_P001 variant of
a GA200x3 protein of Zea mays

AUGAGGCCGCGCCUCCCGCCGAACGUUCCGUCACUCCCCAGCUCCUUGAGUCUCUUGGCGAACUCCCUGAGUAGCCCCGUGAC
CAACACCCCGACUCGGCCCGACAGCUUCCCUGCAUACUUGCAGCUGGCUCACCUAAUGGUGAGCCAGGAACGACAGGAGCCGG
CGGUGCCGUCCAGCUCGUCGUCGUCCGCAAAGCGGGCGGCGACGAGCAUGGACGCAAGCCCGGCCCCGCCGCUCCUGCUGCGU
GCACCCACCCCAUCGCCCUCGAUAGACCUGCCCGCGGGUAAAGACAAAGCGGACGCCGCCGCCUCCAAAGCGGGGGCGGCGGU
GUUCGACCUGCGGCGUGAGCCGAAGAUCCCGGCACCGUUCCUCUGGCCCCAGGAGGAGGCGCGACCCUCGUCGGCCGCAGAGC
UGGAAGUGCCCAUGGUGGACGUAGGCGUGCUGCGAAAUGGGGACCGCGCGGGGCUACGCCGCGCGGCUGCCCAGGUCGCAGCC
GCCUGCGCCACCCAUGGGUUCUUCCAGGUCUGCGGCCACGGGGUCGACGCCGCCCUGGGGCGAGCGGCACUCGACGGGGCCUC
UGACUUCUUCCGCCUACCUUUGGCGGAGAAGCAGAGGGCCCGUCGCGUCCCCGGCACCGUGUCCGGGUACACGAGCGCGCACG
CCGACCGGUUCGCGGCCAAGCUCCCCUGGAAGGAGACCCUGUCGUUCGGCUACCACGACGGCGCCGCGUCGCCUGUCGUCGUG
GACUACUUCGUCGGCACCCUCGGCCAGGAUUUCGAGCCAAUGGGGUGGGUGUACCAGAGGUACUGCGAGGAGAUGAAGGAGCU
GUCGCUGACGAUCAUGGAGCUGCUGGAGCUGAGCCUGGGCGUGGAGCUGCGCGGCUACUACCGGGAGUUCUUCGAGGACAGCC
GGUCCAUCAUGCGGUGCAACUACUACCCGCCGUGCCCGGAGCCGGAGCGCACGCUGGGCACGGGCCCGCACUGCGACCCCACG
GCGCUCACCAUCCUCCUGCAGGACGACGUGGGCGGGCUGGAGGUGCUGGUGGACGGUGAGUGGCGCCCCGUCCGGCCCGUCCC
GGGCGCCAUGGUCAUCAACAUCGGCGACACCUUCAUGGCGCUGUCGAACGGGAGGUACAAGAGCUGCCUGCACCGCGCGGUGG
UGAACCAGCGGCGGGCGCGGCGGUCGCUGGCCUUCUUCCUGUGCCCGCGCGAGGACCGGGUGGUGCGCCCGCCGGCCAGUGCU
GCGCCGCGGCGCUACCCGGACUUCACCUGGGCCGACCUCAUGCGCUUCACGCAGCGCCACUACOGCGCCGACACCCGCACGCU
GGACGCCUUCACCCGCUGGCUCUCCCACGGCCCGGCCCAGGCGGCGGCGCCUCCCUGCACCUAG

Example Trained GA20ox3 Coding Strand DNA Excerpt w/differences from reference highlighted in bold with wavy underline (Template Strand putatively transcribed to primary transcript):

SEQ ID NO: 50 provides a design for a polynucleotide sequence to help identify what needs
DNA editing with CDS trained to a 65.1% expression design level encoding of a
Zm00001eb366090_P001 variant of a GA200x3 protein of Zea mays

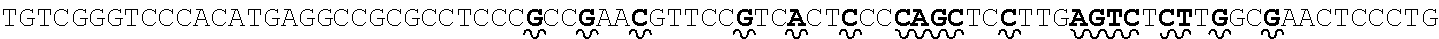

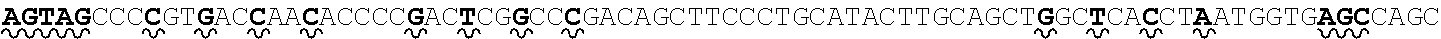

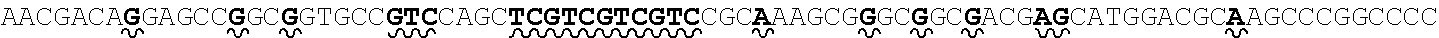

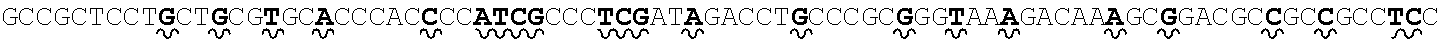

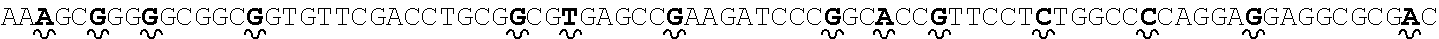

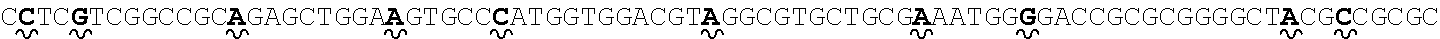

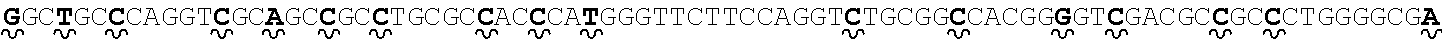

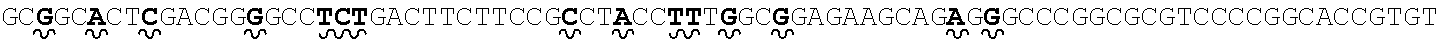

CCGGGTACACGAGCGCGCACGCCGACCGGTTCGCGGCCAAGCTCCCCTGGAAGGAGACCCTGTCGTTCGGCTACCACGACGGC
GCCGCGTCGCCTGTCGTCGTGGACTACTTCGTCGGCACCCTCGGCCAGGATTTCGAGCCAATGGGGTAAGTAAGGTAGTAAGA
AGGAGCGCCGGTTTACATTTACCGCACGTCGGCGTGCGGTCGAGTCGGGACTCGGGAGACGTATGAACCCCCGTCCCGTCCCA
TGCATGTGTGGCAGGTGGGTGTACCAGAGGTACTGCGAGGAGATGAAGGAGCTGTCGCTGACGATCATGGAGCTGCTGGAGCT
GAGCCTGGGCGTGGAGCTGCGCGGCTACTACCGGGAGTTCTTCGAGGACAGCCGGTCCATCATGCGGTGCAACTACTACCCGC
CGTGCCCGGAGCCGGAGCGCACGCTGGGCACGGGCCCGCACTGCGACCCCACGGCGCTCACCATCCTCCTGCAGGACGACGTG
GGCGGGCTGGAGGTGCTGGTGGACGGTGAGTGGCGCCCCGTCCGGCCCGTCCCGGGCGCCATGGTCATCAACATCGGCGACAC
CTTCATGGTAACGAAACGAAAGCGCTCGCTCCTCTGTTTTCCTTGGCCGCTCTTGTCCTGTGTGTATATTCAGTTGAGCTCTC
TCTGTGCTGTTATTTCCCGAATCCTAGTGGACCTAAACGGGCAGGTTATTACAGCACGCACACGTAGGCATGTCATGTAGCTA
GTACATACATAGCGATGCCGATGCAAATGCAATAGAGACATGCGTTCGAGTTGGTTCCTATCTCGGCGGGCTACGGCAGGTAC
ACGCGGCCGCGGCGCGCTCTCTCTAGTCTATCCGCGGCCGCGCCCAGGCCGATCGAGGCTTCCGGGGGAGAGTTGCGACAAGA
GAACGGACCGAGGGGGTCGGCTAGCGGTAGCAAGTTCCCTGTTGGTTTGTGGCGTTGGAGCGTTGCGGAGAGGCTTGCGCGGC
GGCGGGGACGTCGACGGGGACGTGGCGGGGAGACGATACGATGGGTGCCGGGCAGGGCAACGCTTTCGGCGGGTGGCCGTGTC
CAGGTGCGCGCGGCCTTGTCGGTTTCCCCCTCTCGGTGTCCATGGCCGAGAAATGGGTCGACGACCGAGACCGACGCTCGGTG
CGGCGCCCATCCCGTCTGATCCGCCGCGCCACGCGAGCGGCCCTATGCGATGCCGCACGGGCGCGGAGGGCCGTCGCGCGGAG
TATAATGTATAGTATATAGTACAAGGTTGGTTGGAGTCGGGTTGGGTTGGATCGGGTCACCGGTACGTGGTGGCTGCTGTTGC
CCCCGCCGTTTCCGCTTGCACTTTTGTCGCGGTTTCGCTGGCGATCCGGCACGCGGCGCCCACACCACGCCGGGGCTCCAAAC
AGCTCGGGCCCTTGGCCGTGTGGGTGGCAGGCACTTGCACGCGTCCGGTTGTCGCGGCCTGGCCCGCCGCCGGGCGCACCGCA
ACAATGAGACAGCCCGACACGATGATTCTTGTGCACTGTGCTAACCCGCATGCCATGCAGGCGCTGTCGAACGGGAGGTACAA
GAGCTGCCTGCACCGCGCGGTGGTGAACCAGCGGCGGGCGCGGCGGTCGCTGGCCTTCTTCCTGTGCCCGCGCGAGGACCGGG
TGGTGCGCCCGCCGGCCAGTGCTGCGCCGCGGCGCTACCCGGACTTCACCTGGGCCGACCTCATGCGCTTCACGCAGCGCCAC
TACCGCGCCGACACCCGCACGCTGGACGCCTTCACCCGCTGGCTCTCCCACGGCCCGGCCCAGGCGGCGGCGCCTCCCTGCAC
CTAGCGAGCCGGGCCAAGGCCGTCTCTTTCGCCCCACGTGCGCGCCCAGCTGGGCAGGTGGCCAGACACGCGGCCCGCGGGCC
CCGCGCCGCCTTGCCATTTTTTGACGCTGGCCCTACTGCTGTGCTACTAGTGTACATATGCAAGAGTACATATATATATATAT
ATATACGTATTTTCTATATATTATATATAAAAGCAAGGCGGCCCGGTGCCCTTCTCTTGTTTTGTCCACAACTGTTTGATCCC
ATTATTCTATGGACCATGGATACTTCAATGTTTGTACTAAGACCGTGAACGTGGGATTCTTTTCCTTCCTCTGTGTTTTTTCT
GAGAAAAATTAAACTGATTTCTGTGAA

### I. Example 9: Gene Training for Understanding a Popular Gene

In this example, we focus on precisely on modulating expression of an interesting gene in a maize inbred that has been implicated to affect number of silks, ear row number, and flowering time. In this example, the Zm00001eb284010_T001 splice sequence of ZAG1 (i.e. Zea agamous1) from the B73 reference is trained to a higher relative expression design level.

Example Protein Sequence (ZAG1, e.g. Zm00001eb284010_P₀₀₁):

SEQ ID NO: 51 provides the amino acid sequence of the Zm00001eb284010_P001
variant of an ZAG1 protein of Zea mays

MHIREEEATPSTVTGIMSTLTSAGQQKLKEPISPGGGSASVAGSAAERNNGGRGKGKTEIKRIENTTNRQVTFCKRRNGLLKK
AYELSVLCDAEVALIVFSSRGRLYEYANNSVKGTIERYKKATSDNSSAAGTIAEVTIQHYKQESARLRQQIVNLQNSNRALIG
DSITTMSHKELKHLETRLDKALGKIRAKKNDVLCSEVEYMQRREMELQNDNLYLRSRVDENERAQQTANMMGAPSTSEYQQHG
FTPYDPIRSFLQFNIVQQPQFYSQQEDRKDFNDQGGR

Upper Calibrating CDS RNA Sequence (For Calibration via CDSFold, −MFE=559; [0-1] design level=100%):

SEQ ID NO: 52 provides a design for a nucleic acid sequence with CDS trained
to a 100% expression design level encoding of a Zm00001eb284010_P001 variant of a
ZAG1 protein of Zea mays

AUGCAUAUCCGUGAGGAGGAGGCCACGCCUUCUACCGUCACGGGUAUCAUGUCUACACUGACCUCCGCUGGUCAGCAGAAGUU
GAAAGAGCCGAUCUCUCCUGGUGGGGGUAGUGCUUCGGUUGCUGGCAGUGCUGCAGAGAGGAACAAUGGGGGUCGUGGGAAGG
GUAAGACCGAGAUCAAGCGGAUCGAGAACACUACGAACCGCCAGGUCACCUUCUGCAAGAGGCGAAACGGCCUCUUGAAGAAG
GCUUAUGAGCUGUCGGUACUCUGCGACGCGGAGGUGGCCCUCAUCGUGUUCUCGUCGCGGGGCCGCUUGUAUGAGUACGCGAA
CAAUAGUGUCAAAGGCACUAUUGAGCGGUACAAAAAGGCGACCUCCGACAAUAGUUCCGCAGCCGGAACUAUUGCGGAGGUCA
CCAUCCAGCAUUAUAAGCAGGAGUCCGCCCGGCUCCGCCAGCAGAUUGUCAAUCUGCAGAACUCCAAUCGGGCGCUGAUUGGU
GACUCCAUCACUACCAUGAGCCACAAGGAGCUCAAGCACCUUGAGACUCGCUUGGAUAAGGCUCUUGGUAAAAUCCGAGCAAA
GAAGAAUGAUGUUCUUUGCUCGGAGGUGGAGUACAUGCAGCGCCGCGAGAUGGAGCUGCAGAAUGACAAUCUGUAUUUGCGGA
GCCGGGUGGACGAAAACGAGCGGGCCCAGCAGACGGCGAACAUGAUGGGGGCCCCCUCCACGUCGGAGUACCAGCAGCACGGU
UUUACCCCCUACGACCCCAUUCGUUCCUUUCUGCAGUUUAACAUUGUCCAGCAACCGCAGUUCUACUCCCAGCAGGAGGAUCG
GAAAGACUUCAAUGACCAGGGAGGUCGGUGA

Lower Calibrating CDS RNA Sequence (For Calibration via CDSFold, −MFE=87; [0-1] design level=0%):

SEQ ID NO: 53 provides a design for a nucleic acid sequence with CDS trained to a
0% expression design level encoding of a Zm00001eb284010_P001 variant of a ZAG1
protein of Zea mays

AUGCACAUAAGAGAAGAAGAAGCAACACCAUCAACAGUAACAGGAAUAAUGUCAACACUAACAUCAGCAGGACAACAAAAACU
AAAAGAACCAAUAUCACCAGGAGGAGGAUCAGCAUCAGUAGCAGGAUCAGCAGCAGAAAGAAACAACGGAGGAAGAGGAAAAG
GAAAAACAGAAAUAAAAAGAAUAGAAAACACAACAAACAGACAAGUAACAUUCUGCAAAAGAAGAAACGGACUACUAAAAAAA
GCAUACGAACUAUCAGUACUAUGCGACGCAGAAGUAGCACUAAUAGUAUUCUCAUCAAGAGGAAGACUAUACGAAUACGCAAA
CAACUCAGUAAAAGGAACAAUAGAAAGAUACAAAAAAGCAACAUCAGACAACUCAUCAGCAGCAGGAACAAUAGCAGAAGUAA
CAAUACAACACUACAAACAAGAAUCAGCAAGACUAAGACAACAAAUAGUAAACCUACAAAACUCAAACAGAGCACUAAUAGGA
GACUCAAUAACAACAAUGUCACACAAAGAACUAAAACACCUAGAAACAAGACUAGACAAAGCACUAGGAAAAAUAAGAGCAAA
AAAAAACGACGUACUAUGCUCAGAAGUAGAAUACAUGCAAAGAAGAGAAAUGGAACUACAAAACGACAACCUAUACCUAAGAU
CAAGAGUAGACGAAAACGAAAGAGCACAACAAACAGCAAACAUGAUGGGAGCACCAUCAACAUCAGAAUACCAACAACACGGA
UUCACACCAUACGACCCAAUAAGAUCAUUCCUACAAUUCAACAUAGUACAACAACCACAAUUCUACUCACAACAAGAAGACAG
AAAAGACUUCAACGACCAAGGAGGAAGAUAA

Example Reference CDS RNA Sequence (Reference Level, −MFE=272; [0-1] design level=39.2%)

SEQ ID NO: 54 provides the nucleic acid sequence
of the Zm00001eb284010_T001 CDS encoding of the
Zm00001eb284010_P001 variant of a ZAG1 protein
from Zea mays

AUGCACAUCCGAGAAGAGGAGGCUACACCAUCCACAGUAACAGGCAUCAU
GUCGACCCUGACUUCGGCGGGGCAGCAGAAGCUGAAGGAGCCCAUAUCCC
CUGGUGGCGGCUCCGCGUCGGUCGCUGGGUCCGCUGCGGAGAGGAACAAC
GGCGGCAGGGGCAAGGGCAAGACUGAGAUCAAGCGCAUCGAGAACACGAC
CAACAGGCAGGUCACCUUCUGCAAGCGCCGCAACGGCCUCCUCAAGAAGG
CGUACGAGCUCUCCGUGCUCUGCGACGCCGAGGUCGCGCUCAUCGUCUUC
UCCAGCCGCGGCCGCCUCUACGAGUACGCCAA

If desired training [0-1] design level=50%, we might choose to use the Reference CDS RNA Sequence as the Primary Bounding Sequence ([0-1] design level-39.2%) and the Upper Calibrating CDS RNA Sequence ([0-1] design level=100%) as the Ultimate Bounding Sequence ([0-1] design level=100%) for training expression. When using base or prime editing, choosing the Reference CDS RNA Sequence as the Primary Bounding Sequence can reduce the number of polynucleotide positions necessary to edit.

With the PBS at 39.2% and the UBS at 100%, our desired expression level is 50%, which is 17.8% between the selected PBS and UBS levels.

When defining the difference at the triplet (codon) resolution, the triplet indices shown below (one-based indexing) are the set of differences from the PBS to the UBS. In this example, we use the contiguous ordering of the differences.

2 4 5 8 9 10 11 12 13 14 15 18 19 21 22 23 24 28 29 31 32 33 36 37 38 39 41 43 44
46 50 51 52 53 54 56 58 62 66 67 69 76 77 81 84 85 87 88 89 93 95 96 99 101 102 103
106 107 110 112 113 114 115 117 120 122 124 126 128 129 130 132 133 134 136 140 143
146 147 148 149 150 151 154 155 156 157 161 162 163 165 167 168 170 176 177 180 183
184 185 186 188 189 190 191 192 194 196 198 199 200 201 202 203 208 209 210 213 217
218 219 220 221 224 225 226 227 228 229 230 231 233 239 240 241 242 243 245 251 252
253 254 255 256 257 258 259 262 264 265 267 268 271 272 276 281 283 284 285 286 287

Given the difference above, an example of a (contiguous) partial difference PD₈₇ can be described by the triplet indices numbered greater than or equal to 87. This partial difference gives −MFE-319, which is 16.6% between the selected PBS and UBS levels. Another example of a (contiguous) partial difference PD₈₈ can be described by the triplet indices numbered greater than or equal to 88. This second partial difference gives −MFE=324, which is 18.3% between the selected PBS and UBS levels. Together, the window [16.6%, 18.3%] is a narrow window for the example protein with respect to the PBS and UBS, giving us the expression design level narrow window of [49.3%, 50.3%] with respect to the calibrating sequences encoding the protein sequence.

The implied coding sequence ICS(PD₈₈), with trained expression level 50.3%, is shown below with the difference from the Reference CDS RNA Sequence underlined:

SEQ ID NO: 55 provides a design for a nucleic acid sequence with CDS trained
to a 50.3% expression design level encoding of a Zm00001eb284010_P001
variant of a ZAG1 protein of Zea mays

AUGCAUAUCCGUGAGGAGGAGGCCACGCCUUCUACCGUCACGGGUAUCAUGUCUACACUGACCUCCGCUGGUCAGCAGAAGUU
GAAAGAGCCGAUCUCUCCUGGUGGGGGUAGUGCUUCGGUUGCUGGCAGUGCUGCAGAGAGGAACAAUGGGGGUCGUGGGAAGG
GUAAGACCGAGAUCAAGCGGAUCGAGAACACUACGAACCGCCAGGUCACCUUCUGCAAGAGGCGAAACGGCCUCUUGAAGAAG
GCUUAUGAGCUGUCCGUGCUCUGCGACGCCGAGGUCGCGCUCAUCGUCUUCUCCAGCCGCGGCCGCCUCUACGAGUACGCCAA
CAACAGCGUGAAGGGCACCAUUGAGAGGUACAAGAAGGCAACCAGUGACAACUCCAGCGCAGCUGGUACGAUUGCAGAGGUCA
CCAUUCAGCAUUACAAGCAGGAAUCUGCUAGGCUGAGGCAGCAGAUCGUUAACUUGCAGAACUCCAACAGGGCCCUGAUAGGU
GAUUCUAUCACAACCAUGAGCCACAAGGAACUUAAGCACUUGGAGACUAGGUUAGACAAAGCUCUCGGAAAGAUUAGAGCAAA
AAAGAACGAUGUGCUGUGUUCUGAAGUCGAGUACAUGCAGAGAAGGGAAAUGGAGUUGCAGAAUGACAACUUGUACUUAAGGA
GCCGGGUUGAUGAGAAUGAAAGGGCACAACAGACAGCGAACAUGAUGGGGGCACCAUCGACAAGUGAGUAUCAGCAGCACGGU
UUUACUCCUUAUGAUCCAAUAAGGAGCUUCCUGCAGUUCAACAUCGUGCAGCAGCCUCAGUUCUAUUCUCAGCAGGAGGACCG
GAAAGACUUCAACGACCAAGGUGGAAGAUAA

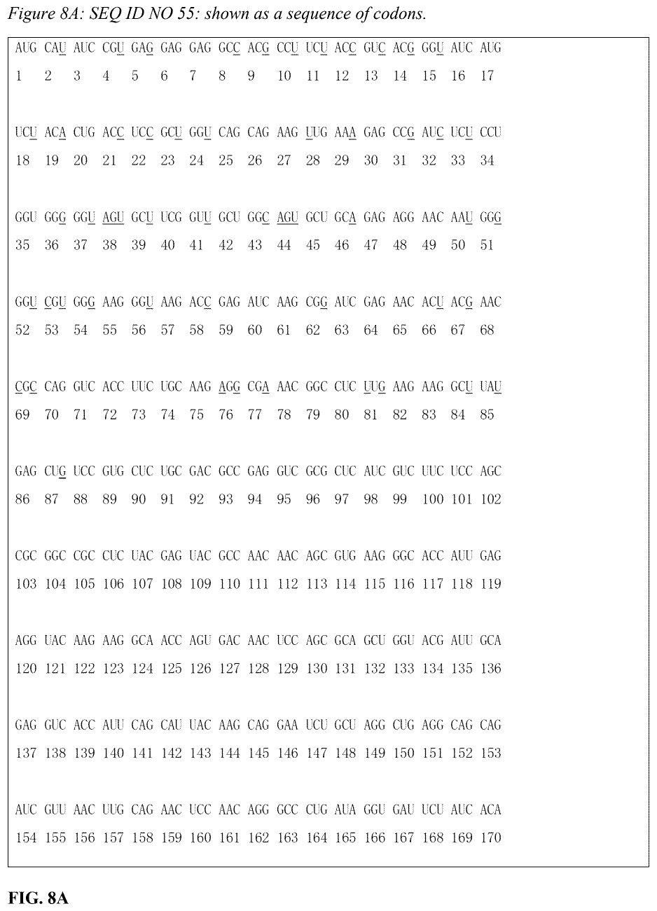

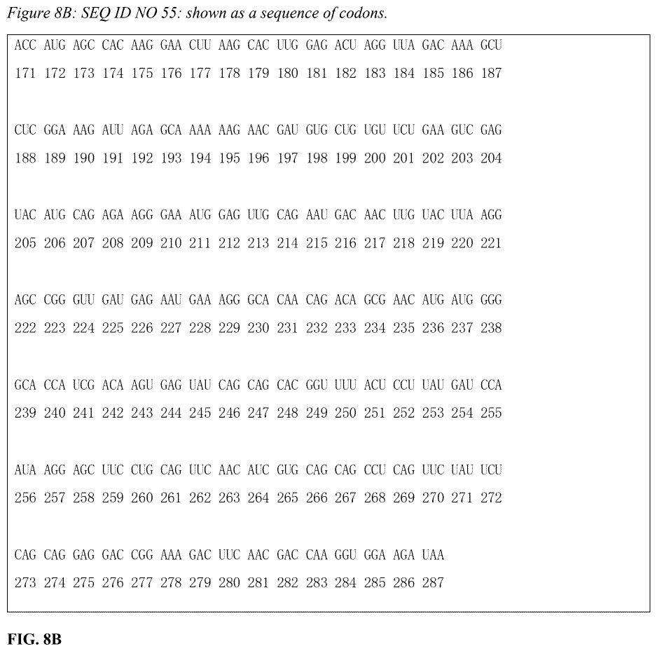

For convenience, FIG. 8 shows the ICS(PD₈₈) sequence SEQ ID NO: 55 with triplets numbered and the difference from reference underlined.

The implied coding sequence ICS(PD₈₇), with trained expression level 49.3%, is shown below with the difference from ICS(PD₈₈) underlined.

SEQ ID NO: 56 provides a design for a nucleic acid sequence with CDS trained
to a 49.3% expression design level encoding of a Zm00001eb284010_P001 variant
of a ZAG1 protein of Zea mays

AUGCAUAUCCGUGAGGAGGAGGCCACGCCUUCUACCGUCACGGGUAUCAUGUCUACACUGACCUCCGCUGGUCAGCAGAAGUU
GAAAGAGCCGAUCUCUCCUGGUGGGGGUAGUGCUUCGGUUGCUGGCAGUGCUGCAGAGAGGAACAAUGGGGGUCGUGGGAAGG
GUAAGACCGAGAUCAAGCGGAUCGAGAACACUACGAACCGCCAGGUCACCUUCUGCAAGAGGCGAAACGGCCUCUUGAAGAAG
GCUUAUGAGCUCUCCGUGCUCUGCGACGCCGAGGUCGCGCUCAUCGUCUUCUCCAGCCGCGGCCGCCUCUACGAGUACGCCAA
CAACAGCGUGAAGGGCACCAUUGAGAGGUACAAGAAGGCAACCAGUGACAACUCCAGCGCAGCUGGUACGAUUGCAGAGGUCA
CCAUUCAGCAUUACAAGCAGGAAUCUGCUAGGCUGAGGCAGCAGAUCGUUAACUUGCAGAACUCCAACAGGGCCCUGAUAGGU
GAUUCUAUCACAACCAUGAGCCACAAGGAACUUAAGCACUUGGAGACUAGGUUAGACAAAGCUCUCGGAAAGAUUAGAGCAAA
AAAGAACGAUGUGCUGUGUUCUGAAGUCGAGUACAUGCAGAGAAGGGAAAUGGAGUUGCAGAAUGACAACUUGUACUUAAGGA
GCCGGGUUGAUGAGAAUGAAAGGGCACAACAGACAGCGAACAUGAUGGGGGCACCAUCGACAAGUGAGUAUCAGCAGCACGGU
UUUACUCCUUAUGAUCCAAUAAGGAGCUUCCUGCAGUUCAACAUCGUGCAGCAGCCUCAGUUCUAUUCUCAGCAGGAGGACCG
GAAAGACUUCAACGACCAAGGUGGAAGAUAA

One skilled in the art shall recognize that one need not allow specific triplets (codons) or other subsequences of importance to change.

Example segment of DNA of Zea Mays B73 inferred from the RNA (primary transcript) is shown below.

SEQ ID NO: 57 provides the polynucleotide sequence of the coding strand of the
DNA region transcribed then spliced to the Zm00001eb284010_T001 transcript with
CDS encoding of a Zm00001eb284010_P001 variant of a ZAG1 protein of Zea mays

GGAGCAAGAAACACTCAGAAGCTGCCCAGAGCTACCACCCTTCTTATCCCCACCCCTCCTCCTCCTACCTTTTCTCCTTCAGA
CCTCAAAATCTGTGTGTCTCCTGCCGCGGCTAGCTGATAGGAACAAGAGCATGCACATCCGAGAAGAGGAGGCTACACCATCC
ACAGTAACAGGCATCATGGTGATTTCTTGTTCTTCCCATTGGCTTCTTTAATTTGGAGCAGATCCACCTCTCTGTGTCGTTGT
CGTCTCCTAGCAATTCTTGAGCGTGGTTCTCTTGTGTGCAGTCGACCCTGACTTCGGCGGGGCAGCAGAAGCTGAAGGAGCCC
ATATCCCCTGGTGGCGGCTCCGCGTCGGTCGCTGGGTCCGCTGCGGAGAGGAACAACGGCGGCAGGGGCAAGGGCAAGACTGA
GATCAAGCGCATCGAGAACACGACCAACAGGCAGGTCACCTTCTGCAAGCGCCGCAACGGCCTCCTCAAGAAGGCGTACGAGC
TCTCCGTGCTCTGCGACGCCGAGGTCGCGCTCATCGTCTTCTCCAGCCGCGGCCGCCTCTACGAGTACGCCAACAACAGGTCT
CTTCTTTCTCTCCCCAGCTCTGTGCATCTCTCTCTCTGTGTGGTACTAGCTAGCTAGTACCGCAATATAATGGTAGTAGGCGC
TACTACGACGAATCACCTTCGCTCCAAGAAATGGTAGGCTACATATATAAGTGATGAGTTTGTCGTCCCGTCTAGCTAGCTAG
ACACATGATGTGTATTTCGACGACGCTCTCCGAGAATTGAAGCAGCAAGTTAGCTCCCAGCTTTCTTGCTTGCAAAGAGGAAA
GCTGCATCTCGTTTGCTGCTACGACTACATGCGTTCTGTTTATTAACTGTCTTGCTTATTTTTTGTGCTACCGCGGAGAGGGG
CGAAAGAAATCGCAACGGTTTCTTTGCGCGCGTGCGTGCTCCGTCATCCTTTCCGGTTTCTCTTGTGTTCCTGTGAGGTGAGC
TAGATGTCTAGATAGATTCAGCGCAGGGGTATGCTTTGTGAGGTGAGCTTTTCTAGTGCGCGTGCGTGCAATGGGCGGGTGCC
TTGCGGTTGCTTTGGTTTGGTGTGGTGGTCCGCTTCTTTTCTTGCTCCATTTAATTATTTCTTTCCTTTCTATGATCTATATA
TCCCTGAGGAATATCAGTGAAGACTTATTAGATCTCTCATACCTCATGATACATAGCAAGCATTGTTCTCACACAAGGAAAAA
AAACTTTTTGTTGCTTTGGTCATGTATATATGTGCAGATCTAGTAATTATTATCCTTCGCTCTTAGATTTGGGTCACTAGCTG
GATGTGCGCGCGTGCAGTAACTCGGTAACTGGATGCAAACTTGTGAATGTGAGTGAGGGGAGGTAAGCATTTGAGACGTAAAA
AGATGCCAGGCGCGCATCCTGTCCTGCTAGTGCTATATGATGTTAGGGTTAGGGTTCCATCCCATGTGCGTCCGTCGTCATCA
TCACCATCTGGCCTCTCTCTCTCTCTTCTCTCTCTCTCTCTCTCTCTCTCTCTCTCTCTCTCTCTCTCATGCTGTCATACATT
GTGTTTTCTGTAGATGTTACTGTTGTCCCTGCGTGTGTGGTGGTTCTGCAGGTCTCCTCGTCACAGCCATGTATATCTGCGCG
CGCGCTGTGCACTGTGCTGCATATGCATATGCTAAAGTGATGCGCAACTGCCCATCACGGGAGCTCTGTCCTCTCTGCCACCA
CATAGCACTGTAGTTTTTAGGACTGGCAGGAAATAAAAGGGGGGTAATGTAATGCAGAAGGAAACGATAACATCAAATTGAAA
ACAAAGAAGCAACCTAGAAACAGCGTGATAGCAAATTAAATTTCCTTAGGCATTTGTGCTTATTTTTTAAGATTTCTTTATCA
CATCCTTATGTTGCACACGTACATGCAATGCAAGTGGCATGAGTGAACGAATATATAGATTCATTACAGCTTTGACAAATTGG
AACCACTCCATCTGATTGAATGCGTTTGACTTGTAAACTCAAAGGACAATATGTGTTGTGTTTCTTCGCAGAAATTTGAGCAT
TATGATTGGGTTCTCGTATCTTCAAGTGAATATTTACTGTTAATAGTAATAGTAGTGTTCATCACACTGAACCATAAATTTGA
GAGAAACACCATTTTGTAAGTAATCAAATTACTTTTCTTGAACAGATTCCAAAAGAATCAAAATTTCATCTATATTGTTGCAA
TATTGAAACTAAAAAGTAGCTCGGATGCATTAGTCAACATGGTATTGCAAATATTTGTACAATAAAATCATAGTTTTGAAACT
GTTGTATCTATATGGTGTTTGATTGGTTACTTGTGTTTGCAATGTATTCAGGCAGTGCAGATTGGTTGTATTTGGTTTGCCAG
ACATATATAGTCTCTTATTCATGCTTGCACTTTGCTCAAAGAAAGCACAATAATGCATATTAGCTAGTATGCCAGATTTGGTC
GTACTTAGACAAGGTTTGCACAATCTGATGGGATGGTCATACAAAAATAAAACAACCTCTTCCCTTTACATATACTAAAAATA
TGAGAACTCAAATAGAACATGGATCGGAGGTAGCAATAAAGTGTGAAGAACCAAACATATTTTTTATACACTGCATGTGTTTC
TGATCGACTCAGGTTGCATTGCATAAGGTCTAATGCAGAGAAGACTAATGCAAGCAAACAATTAGAGCCATAACATCTTATAA
TTGATATAAAAGTATACCTATCTATTTCATGATAATTTTAAATATAAAAGGTTTGCTCAAATAAAAATGCAAACTATAATTGA
TTTTTTTTTTGCTCAAAGCTCGACGGAAGCCAGATGTTCGAACGGGTGTAGCTGCAATTCATAGAGATAAAACACTGTAGAAA
TATTAGAGCGACCTGTACGAATTAAAGTCAATCGACGCTATAGATATATATTTGGTCTTTGAGGGCGTACGGGGCAACTGAAA
TGTGTGCCAATTTGTTTGTGTGATGTGAATGCTCTGTATACGGAACCAGTAGCATGACCAGTTTTCCTTGCCACTGCCAGAGT
TGTATCGGGGGACTACAAGGGGAAGAAAGCTGGCAATGCAAGCTGGTGTTGGTCAGAAGGGTTATGGCACCCAACGGTAGGGC
TGACCAGTTGGGTAACAGCTGAGTCCATGTCTTATCTATGCAGCACTCCTTTGTGACCAATGGGTAGTAGATGTCCACTGCTT
GCAGGCAATCATGTTGTGGGTAATGCATGGCTACTGCCAAGCATGCTCGAGGATGCTAAGGTTTTTGCTATGTTCACCTACAG
AGTACAGAACAGCAGTACCTTTGAAATTGGTAGCCATCCGTCTCAAAATTCGTTCTCATTTTCGAAAATGCAACCATTTTAAA
CTTACAAATATATATAATAAAATATTAATATTACATAATTAGTATCGTTAGAAGATCTTTGAATCTATTTTCATAACAAATAT
TTTTAAAGATACAAATATTACAAATATTTTTTTATAAATTTAAATCAGTCTTGACAAACTTTGACCTGCACGAAATGACTTAG
ACAACTATTTTAGAATGGAGAGAGCATATAAATTATGTGAGTCAAAGAGTTTCTTAGAAATAATGGTTGGCACTAAGTGGGTT
CTACATTATTGCCCCCATAAATATTTCTTTGAAGCACTGCTATTAAAATGCTTGGCCTCCGCGATGGATGTTATGAGTTCCAA
AAAAAGTGACGAGTTCCTGTTAATCTGTGTATGGCAACTGCAAAATTGGCATGTTTAGAAAATACATATCTCGTGTAAGCATA
GGGTTCAAATTTGAGTCAAGATGCATGATTCATGTCTCATATTTCTTTTAGCTAGAATAAAAGTGGTATTTACATTTCCTTTT
TGAACGTAAGACATTTACATGGATATGTCATCTTTTTCTGACAAATATTTAGACAACTGTTTTTTACCATCAAAGCTAATTAA
TGTGAATAACACATCATTTTTTAGATTAGATTGTTTAATTTTTTGCAATGAAAAAATCATTCCTTTGAAGAATCATGTGATCC
ATCTTTCTCCTTTCACTTCCGTTGCACCATTAACATAAGTTATTTAACAATACAATCCATATGTTCCTTTAAATTTGTTTATG
TCCTATTTAGTAAAAATATCCACTTAAGCAACACAAAGAGAGCTGGCTCAACTTAACTGTGAATGCATCGAATCCACTAACTA
TTGCGCTTTTGGTTATTATCAACAACAAATATTTTTATTAATATATCAAGATAATCTATATTTTATCAGTTTCTCAATGTTTC
AAGATTTAAAAATATAGGTCTTGAGTTTTTTTTCTTGAGAGGGTAATTTTACCAGGATGTTTTTTGGTTCAGTTCAAAATTTA
ATTCAATCCTAATGTTTAGATCTAGACCCCCTTTTTTTAAACAGTTCTTGTAGGATTTGACTTTGTTGAAGAAAACAAGGATG
AATTTTTTTGCTATGCTAGCTTAGGAGTTCCGATTTTCTTAAAAAGAACTGTAGTAGTTATTAGGTGTGGTATTACATAGGTT
CAAACTCCATGTATGTGCACTACAAACATATTATGGGTCTGTTTGGTTTAGCTGTAGATTCCTAAAACTCTGATTCTAGCTAT
GAGCTGTGAGAAAGCTACTATGAACTGACAATTATGAGAAAACTGAAATTAATTTGGCTGAAACAACTGTCAACTATAGATTC
TATAAGAGCATGACAGCTTGTAACATAGACATCATGAATCCATAAAATCTAGGGTAGGAGGTGATTCTGAATTGAACTAGAGG
AATCTATAGTTTATCTATTCTACTATTGTATATGTTTGATTGAGATTTCAGATTTTGGACAGCAAATCTAGTTCAGAAAGCTA
AACCAAACATACCATATAAGAACTCAAGTTCTATGTAGATGCATCAGTATTTATTAGAATGCTAACTCAATGGCAGTTAGGAT
TTGATTATGCTGGCAATGCGATAGTATACAATATTAGACTATTAGTGAAGAGTGAAGCAGTAATGTAAGCAATGCATGTGCTT
ACTAAAACGTGATAAATCATTGTTGCACCGAATATTAATACACAACATTAGGATGTGCTTGCACTTCCTGTTATTATCTGTAG
CATTGACGAGGACCACTCTCTATTTTCACATGACACTAAATAATGTGTTAAAATCAACTACTTCTAAGTGATTGCAGCAGTAT
TTACCACACCAAATTAATATCACAAAATAGATTATGAAATATATTTTCATAATTTAACCATTTAGAGTCCTAGATGATGTTAC
TATTTAAGTGATTGCTGGGATGGCTTTACTTTAGCATGGAAGTATTAATAGTATTTTGGGACGATGCGTTAGAATATTGGTTA
CTTTATGTGCTGATTGTATTGACTTTCACTAATGGGGCTGTTCTGGCTAGATTATTAAAGCCGTTCGGACTGCTGCTGCCGTA
TAGACAAAATACTGTAGAAAAGGTAGAAGCCGGTGGATTAAAGCCGAAATACTGTAGAAAAAGTAGAAGCCGGTGGATTAAAG
CCGCAACGGACAGGATGAATGTCAAATTGTAATCGACATACTTGCTAGAAGGCAATGTATCCATTATAATCGCTGGAAGATGT
CAAATTTCAGTTATTGTATATGTTCTATGAATCTCCATTATAATTTATTATGCTTGTGACGATTGTTCTATTATCTGTTGTAT
TTTTTCAAGAAAAATATCTAGTTTTGCCCTGGAACAATTTTGTTTCATATTTTCATTCTTTCGGCACATACAATTATCTCACC
TGATTGAATGTATGTGTACTAACTACTAACTACAGTATTATATTGTATGTACAGCGTGAAGGGCACCATTGAGAGGTACAAGA
AGGCAACCAGTGACAACTCCAGCGCAGCTGGTACGATTGCAGAGGTCACCATTCAGGTAACATCATGTCGATCAGTCAACACA
TCGTTTCTGATCCTACAAGACCCTAGTCGTAGTGTTGGCTACCACAGGCTATGCTGTCTATTGATGACCCTAGTGTTGTACTA
TGCAACATTCCTTGCAACTGTGTGCTCTACACAGCCAAAATATTTTCTCTGGGCGAAATCTTCAGACTGGACATGCACCCTGT
GGGTTCTGGGCATTACACGGCAGAAATTCCAGTGACGATACTAAGAGAACCTGCTTGGACCAGCATTTCATGTGGAGTTGTGC
AGATATATAATTGTCCTCTCTGGATGTCTGGGTAGCTTTGGTTATTCCATGCTCCAAATGGTAGAGAGGATAGCATGCATGTT
TTGGACAAAAACGAATAAAACCACTGAAGGCTAGTAGGAAAATTCTTCTTGATGGGTTTTCGTAAGATGTGGCAATTATAATA
TTCCTCTGATAAATATATATATGGCTAGTAGGAAACTTTATAAGTATATATATGGCAGTCAGTTTGGTGAAATGGAGTTTTAG
TGTTGCACATATTTTCTTTTCCCTGAAATTGATTTCGATGAATAAATTAATTGGACTACTTTTGTTTTTTCTTGCAATTGAGA
AATGAGTACACCATTTAATTCACATTTTGCAGTCGGACCTGTTTGTTATGAATGTCAATATGGAACATCCACTAACAATTTGG
ATAGAAACCAACAGCAGCATGAAATATTACTAAATGAACTTTTGCAAACTTGCAGCATTACAAGCAGGAATCTGCTAGGCTGA
GGCAGCAGATCGTTAACTTGCAGAACTCCAACAGGTTCAGTTCTTATAGAAAATTATCATGTGCGCGAGAATACAGAAAGGTC
ATTTGATGAACTTATAAGTTTTCAGGGCCCTGATAGGTGATTCTATCACAACCATGAGCCACAAGGAACTTAAGCACTTGGAG
ACTAGGTTAGACAAAGCTCTCGGAAAGATTAGAGCAAAAAAGGTACTACGTGCTATATATCTATAATAATCATATTTACTGCA
TTATGTGTGGAAAAATAAATATAGTATTTCCTTTTACGCAATTAATGCGGATCAAACTCTGCAGAACGATGTGCTGTGTTCTG
AAGTCGAGTACATGCAGAGAAGGGTAAACAGCAACAAGAACACATTTTTCCTCTCACGAACTTATTGATTTACTCGACATTGA
ACAAAATAAAAACAAGAATTTCGCATTTTCAGGAAATGGAGTTGCAGAATGACAACTTGTACTTAAGGAGCCGGGTAAGTAAC
CTGTAGTTGCTAATTAAGAAGGACAGTCTTAGAAGCAAAATATGGAACTGTAATTACTAAAATGCCAAGTTTACCTTAACTTT
GAAAATAAAAAATAGTGTAACAATTTAACTGTCTCACAATGCAAGCATAGGTTGATGAGAATGAAAGGGCACAACAGACAGCG
AACATGATGGGGGCACCATCGACAAGTGAGTATCAGCAGCACGGTTTTACTCCTTATGATCCAATAAGGAGCTTCCTGCAGTT
CAACATCGTGCAGCAGCCTCAGTTCTATTCTCAGCAGGAGGACCGGAAAGACTTCAACGACCAAGGTAGATTTTTTTTTTATA
TCTACCACATTTATTTGAAATGTATTTAAATGGACACCCTAAGGTATTTCATAATACCATGTATATTTTTCTAAATGAAATAA
TAATCCCTCCATCTCAAATTATTATTCATTTTAATTCTTGGTTTTTAAGAATGATGATGAATCTAGACACATACATCAATTAT
TAACTGTACGAATCTATTAGAAGTTTAAAATGAATTTTAATTTGGGACAGAGACAATATTTATTAGTCATCACAAAACTTGGA
AGCCATAGGGCTAGGAAAACAAAATCATTGCAGTTAGTTTTAAATAAAATGCCTAAACCTTCCACTTGGCTATATATATATAT
ATATATATATATATATATATATATATATATATATATATATATATATATATATATATATATATATATATATATATATATATATA
TATATATATATATATATATATATATATATATAATTTCCTCTCCAAGTACATGTTCTGCTATTCTGATTCCTTTTAATGTCTTT
AGGTGGAAGATAAATATTGGACCTCTCAAGCTTCAGTACTTATCCGTGATGATGCATGACTGCCAGTGAGAAACTGAGCTACA
TTACTGTGGAATTATATGTAAAGACTAGTACTACTGCTTCATATATGTGCATGTGCGCACACGCGCACGTAGTATGCACAATT
TCATCCCACTATTATGGCTTGGCACCAACTATGTCTCCTTAATTATCATAGGAGAAAAATAAGTCACGCACAAAAAAATTCTA
AAGATGAGGCGCAGTGTTGGATGACTGAAACTGAAAGATAGGATTCTCGTGGATGGCGGTGGTCATATATATGTCATGCACAT
CTGATCTCTGTTCCAAAATGCCACATCTTCTTTCCCAACCCACTCGAATCTACACCGAGAATATATATGTTTTATTCGTAATG
TATAGACACTATTAGCAATGATACTGTGAACTATGGTTATGGTCCACAATAAGAAATGCATATTTACCTGTG

Given the B73 DNA and the CDS (i.e. the Reference RNA above), a mapping via an alignment between relevant section of DNA and the CDS sequences is shown via underlining in sequence below. The concatenation of the underlined subsequences forms the CDS.

TABLE 1
SEQ ID NO: 57

GGAGCAAGAAACACTCAGAAGCTGCCCAGAGCTACCACCCTTCTTATCCCCACCCCTCCTCCTCCTACCTTTTCTCCTTCAGA
CCTCAAAATCTGTGTGTCTCCTGCCGCGGCTAGCTGATAGGAACAAGAGCATGCACATCCGAGAAGAGGAGGCTACACCATCC
ACAGTAACAGGCATCATGGTGATTTCTTGTTCTTCCCATTGGCTTCTTTAATTTGGAGCAGATCCACCTCTCTGTGTCGTTGT
CGTCTCCTAGCAATTCTTGAGCGTGGTTCTCTTGTGTGCAGTCGACCCTGACTTCGGCGGGGCAGCAGAAGCTGAAGGAGCCC
ATATCCCCTGGTGGCGGCTCCGCGTCGGTCGCTGGGTCCGCTGCGGAGAGGAACAACGGCGGCAGGGGCAAGGGCAAGACTGA
GATCAAGCGCATCGAGAACACGACCAACAGGCAGGTCACCTTCTGCAAGCGCCGCAACGGCCTCCTCAAGAAGGCGTACGAGC
TCTCCGTGCTCTGCGACGCCGAGGTCGCGCTCATCGTCTTCTCCAGCCGCGGCCGCCTCTACGAGTACGCCAACAACAGGTCT
CTTCTTTCTCTCCCCAGCTCTGTGCATCTCTCTCTCTGTGTGGTACTAGCTAGCTAGTACCGCAATATAATGGTAGTAGGCGC
TACTACGACGAATCACCTTCGCTCCAAGAAATGGTAGGCTACATATATAAGTGATGAGTTTGTCGTCCCGTCTAGCTAGCTAG
ACACATGATGTGTATTTCGACGACGCTCTCCGAGAATTGAAGCAGCAAGTTAGCTCCCAGCTTTCTTGCTTGCAAAGAGGAAA
GCTGCATCTCGTTTGCTGCTACGACTACATGCGTTCTGTTTATTAACTGTCTTGCTTATTTTTTGTGCTACCGCGGAGAGGGG
CGAAAGAAATCGCAACGGTTTCTTTGCGCGCGTGCGTGCTCCGTCATCCTTTCCGGTTTCTCTTGTGTTCCTGTGAGGTGAGC
TAGATGTCTAGATAGATTCAGCGCAGGGGTATGCTTTGTGAGGTGAGCTTTTCTAGTGCGCGTGCGTGCAATGGGCGGGTGCC
TTGCGGTTGCTTTGGTTTGGTGTGGTGGTCCGCTTCTTTTCTTGCTCCATTTAATTATTTCTTTCCTTTCTATGATCTATATA
TCCCTGAGGAATATCAGTGAAGACTTATTAGATCTCTCATACCTCATGATACATAGCAAGCATTGTTCTCACACAAGGAAAAA
AAACTTTTTGTTGCTTTGGTCATGTATATATGTGCAGATCTAGTAATTATTATCCTTCGCTCTTAGATTTGGGTCACTAGCTG
GATGTGCGCGCGTGCAGTAACTCGGTAACTGGATGCAAACTTGTGAATGTGAGTGAGGGGAGGTAAGCATTTGAGACGTAAAA
AGATGCCAGGCGCGCATCCTGTCCTGCTAGTGCTATATGATGTTAGGGTTAGGGTTCCATCCCATGTGCGTCCGTCGTCATCA
TCACCATCTGGCCTCTCTCTCTCTCTTCTCTCTCTCTCTCTCTCTCTCTCTCTCTCTCTCTCTCTCTCATGCTGTCATACATT
GTGTTTTCTGTAGATGTTACTGTTGTCCCTGCGTGTGTGGTGGTTCTGCAGGTCTCCTCGTCACAGCCATGTATATCTGCGCG
CGCGCTGTGCACTGTGCTGCATATGCATATGCTAAAGTGATGCGCAACTGCCCATCACGGGAGCTCTGTCCTCTCTGCCACCA
CATAGCACTGTAGTTTTTAGGACTGGCAGGAAATAAAAGGGGGGTAATGTAATGCAGAAGGAAACGATAACATCAAATTGAAA
ACAAAGAAGCAACCTAGAAACAGCGTGATAGCAAATTAAATTTCCTTAGGCATTTGTGCTTATTTTTTAAGATTTCTTTATCA
CATCCTTATGTTGCACACGTACATGCAATGCAAGTGGCATGAGTGAACGAATATATAGATTCATTACAGCTTTGACAAATTGG
AACCACTCCATCTGATTGAATGCGTTTGACTTGTAAACTCAAAGGACAATATGTGTTGTGTTTCTTCGCAGAAATTTGAGCAT
TATGATTGGGTTCTCGTATCTTCAAGTGAATATTTACTGTTAATAGTAATAGTAGTGTTCATCACACTGAACCATAAATTTGA
GAGAAACACCATTTTGTAAGTAATCAAATTACTTTTCTTGAACAGATTCCAAAAGAATCAAAATTTCATCTATATTGTTGCAA
TATTGAAACTAAAAAGTAGCTCGGATGCATTAGTCAACATGGTATTGCAAATATTTGTACAATAAAATCATAGTTTTGAAACT
GTTGTATCTATATGGTGTTTGATTGGTTACTTGTGTTTGCAATGTATTCAGGCAGTGCAGATTGGTTGTATTTGGTTTGCCAG
ACATATATAGTCTCTTATTCATGCTTGCACTTTGCTCAAAGAAAGCACAATAATGCATATTAGCTAGTATGCCAGATTTGGTC
GTACTTAGACAAGGTTTGCACAATCTGATGGGATGGTCATACAAAAATAAAACAACCTCTTCCCTTTACATATACTAAAAATA
TGAGAACTCAAATAGAACATGGATCGGAGGTAGCAATAAAGTGTGAAGAACCAAACATATTTTTTATACACTGCATGTGTTTC
TGATCGACTCAGGTTGCATTGCATAAGGTCTAATGCAGAGAAGACTAATGCAAGCAAACAATTAGAGCCATAACATCTTATAA
TTGATATAAAAGTATACCTATCTATTTCATGATAATTTTAAATATAAAAGGTTTGCTCAAATAAAAATGCAAACTATAATTGA
TTTTTTTTTTGCTCAAAGCTCGACGGAAGCCAGATGTTCGAACGGGTGTAGCTGCAATTCATAGAGATAAAACACTGTAGAAA
TATTAGAGCGACCTGTACGAATTAAAGTCAATCGACGCTATAGATATATATTTGGTCTTTGAGGGCGTACGGGGCAACTGAAA
TGTGTGCCAATTTGTTTGTGTGATGTGAATGCTCTGTATACGGAACCAGTAGCATGACCAGTTTTCCTTGCCACTGCCAGAGT
TGTATCGGGGGACTACAAGGGGAAGAAAGCTGGCAATGCAAGCTGGTGTTGGTCAGAAGGGTTATGGCACCCAACGGTAGGGC
TGACCAGTTGGGTAACAGCTGAGTCCATGTCTTATCTATGCAGCACTCCTTTGTGACCAATGGGTAGTAGATGTCCACTGCTT
GCAGGCAATCATGTTGTGGGTAATGCATGGCTACTGCCAAGCATGCTCGAGGATGCTAAGGTTTTTGCTATGTTCACCTACAG
AGTACAGAACAGCAGTACCTTTGAAATTGGTAGCCATCCGTCTCAAAATTCGTTCTCATTTTCGAAAATGCAACCATTTTAAA
CTTACAAATATATATAATAAAATATTAATATTACATAATTAGTATCGTTAGAAGATCTTTGAATCTATTTTCATAACAAATAT
TTTTAAAGATACAAATATTACAAATATTTTTTTATAAATTTAAATCAGTCTTGACAAACTTTGACCTGCACGAAATGACTTAG
ACAACTATTTTAGAATGGAGAGAGCATATAAATTATGTGAGTCAAAGAGTTTCTTAGAAATAATGGTTGGCACTAAGTGGGTT
CTACATTATTGCCCCCATAAATATTTCTTTGAAGCACTGCTATTAAAATGCTTGGCCTCCGCGATGGATGTTATGAGTTCCAA
AAAAAGTGACGAGTTCCTGTTAATCTGTGTATGGCAACTGCAAAATTGGCATGTTTAGAAAATACATATCTCGTGTAAGCATA
GGGTTCAAATTTGAGTCAAGATGCATGATTCATGTCTCATATTTCTTTTAGCTAGAATAAAAGTGGTATTTACATTTCCTTTT
TGAACGTAAGACATTTACATGGATATGTCATCTTTTTCTGACAAATATTTAGACAACTGTTTTTTACCATCAAAGCTAATTAA
TGTGAATAACACATCATTTTTTAGATTAGATTGTTTAATTTTTTGCAATGAAAAAATCATTCCTTTGAAGAATCATGTGATCC
ATCTTTCTCCTTTCACTTCCGTTGCACCATTAACATAAGTTATTTAACAATACAATCCATATGTTCCTTTAAATTTGTTTATG
TCCTATTTAGTAAAAATATCCACTTAAGCAACACAAAGAGAGCTGGCTCAACTTAACTGTGAATGCATCGAATCCACTAACTA
TTGCGCTTTTGGTTATTATCAACAACAAATATTTTTATTAATATATCAAGATAATCTATATTTTATCAGTTTCTCAATGTTTC
AAGATTTAAAAATATAGGTCTTGAGTTTTTTTTCTTGAGAGGGTAATTTTACCAGGATGTTTTTTGGTTCAGTTCAAAATTTA
ATTCAATCCTAATGTTTAGATCTAGACCCCCTTTTTTTAAACAGTTCTTGTAGGATTTGACTTTGTTGAAGAAAACAAGGATG
AATTTTTTTGCTATGCTAGCTTAGGAGTTCCGATTTTCTTAAAAAGAACTGTAGTAGTTATTAGGTGTGGTATTACATAGGTT
CAAACTCCATGTATGTGCACTACAAACATATTATGGGTCTGTTTGGTTTAGCTGTAGATTCCTAAAACTCTGATTCTAGCTAT
GAGCTGTGAGAAAGCTACTATGAACTGACAATTATGAGAAAACTGAAATTAATTTGGCTGAAACAACTGTCAACTATAGATTC
TATAAGAGCATGACAGCTTGTAACATAGACATCATGAATCCATAAAATCTAGGGTAGGAGGTGATTCTGAATTGAACTAGAGG
AATCTATAGTTTATCTATTCTACTATTGTATATGTTTGATTGAGATTTCAGATTTTGGACAGCAAATCTAGTTCAGAAAGCTA
AACCAAACATACCATATAAGAACTCAAGTTCTATGTAGATGCATCAGTATTTATTAGAATGCTAACTCAATGGCAGTTAGGAT
TTGATTATGCTGGCAATGCGATAGTATACAATATTAGACTATTAGTGAAGAGTGAAGCAGTAATGTAAGCAATGCATGTGCTT
ACTAAAACGTGATAAATCATTGTTGCACCGAATATTAATACACAACATTAGGATGTGCTTGCACTTCCTGTTATTATCTGTAG
CATTGACGAGGACCACTCTCTATTTTCACATGACACTAAATAATGTGTTAAAATCAACTACTTCTAAGTGATTGCAGCAGTAT
TTACCACACCAAATTAATATCACAAAATAGATTATGAAATATATTTTCATAATTTAACCATTTAGAGTCCTAGATGATGTTAC
TATTTAAGTGATTGCTGGGATGGCTTTACTTTAGCATGGAAGTATTAATAGTATTTTGGGACGATGCGTTAGAATATTGGTTA
CTTTATGTGCTGATTGTATTGACTTTCACTAATGGGGCTGTTCTGGCTAGATTATTAAAGCCGTTCGGACTGCTGCTGCCGTA
TAGACAAAATACTGTAGAAAAGGTAGAAGCCGGTGGATTAAAGCCGAAATACTGTAGAAAAAGTAGAAGCCGGTGGATTAAAG
CCGCAACGGACAGGATGAATGTCAAATTGTAATCGACATACTTGCTAGAAGGCAATGTATCCATTATAATCGCTGGAAGATGT
CAAATTTCAGTTATTGTATATGTTCTATGAATCTCCATTATAATTTATTATGCTTGTGACGATTGTTCTATTATCTGTTGTAT
TTTTTCAAGAAAAATATCTAGTTTTGCCCTGGAACAATTTTGTTTCATATTTTCATTCTTTCGGCACATACAATTATCTCACC
TGATTGAATGTATGTGTACTAACTACTAACTACAGTATTATATTGTATGTACAGCGTGAAGGGCACCATTGAGAGGTACAAGA
AGGCAACCAGTGACAACTCCAGCGCAGCTGGTACGATTGCAGAGGTCACCATTCAGGTAACATCATGTCGATCAGTCAACACA
TCGTTTCTGATCCTACAAGACCCTAGTCGTAGTGTTGGCTACCACAGGCTATGCTGTCTATTGATGACCCTAGTGTTGTACTA
TGCAACATTCCTTGCAACTGTGTGCTCTACACAGCCAAAATATTTTCTCTGGGCGAAATCTTCAGACTGGACATGCACCCTGT
GGGTTCTGGGCATTACACGGCAGAAATTCCAGTGACGATACTAAGAGAACCTGCTTGGACCAGCATTTCATGTGGAGTTGTGC
AGATATATAATTGTCCTCTCTGGATGTCTGGGTAGCTTTGGTTATTCCATGCTCCAAATGGTAGAGAGGATAGCATGCATGTT
TTGGACAAAAACGAATAAAACCACTGAAGGCTAGTAGGAAAATTCTTCTTGATGGGTTTTCGTAAGATGTGGCAATTATAATA
TTCCTCTGATAAATATATATATGGCTAGTAGGAAACTTTATAAGTATATATATGGCAGTCAGTTTGGTGAAATGGAGTTTTAG
TGTTGCACATATTTTCTTTTCCCTGAAATTGATTTCGATGAATAAATTAATTGGACTACTTTTGTTTTTTCTTGCAATTGAGA
AATGAGTACACCATTTAATTCACATTTTGCAGTCGGACCTGTTTGTTATGAATGTCAATATGGAACATCCACTAACAATTTGG
ATAGAAACCAACAGCAGCATGAAATATTACTAAATGAACTTTTGCAAACTTGCAGCATTACAAGCAGGAATCTGCTAGGCTGA
GGCAGCAGATCGTTAACTTGCAGAACTCCAACAGGTTCAGTTCTTATAGAAAATTATCATGTGCGCGAGAATACAGAAAGGTC
ATTTGATGAACTTATAAGTTTTCAGGGCCCTGATAGGTGATTCTATCACAACCATGAGCCACAAGGAACTTAAGCACTTGGAG
ACTAGGTTAGACAAAGCTCTCGGAAAGATTAGAGCAAAAAAGGTACTACGTGCTATATATCTATAATAATCATATTTACTGCA
TTATGTGTGGAAAAATAAATATAGTATTTCCTTTTACGCAATTAATGCGGATCAAACTCTGCAGAACGATGTGCTGTGTTCTG
AAGTCGAGTACATGCAGAGAAGGGTAAACAGCAACAAGAACACATTTTTCCTCTCACGAACTTATTGATTTACTCGACATTGA
ACAAAATAAAAACAAGAATTTCGCATTTTCAGGAAATGGAGTTGCAGAATGACAACTTGTACTTAAGGAGCCGGGTAAGTAAC
CTGTAGTTGCTAATTAAGAAGGACAGTCTTAGAAGCAAAATATGGAACTGTAATTACTAAAATGCCAAGTTTACCTTAACTTT
GAAAATAAAAAATAGTGTAACAATTTAACTGTCTCACAATGCAAGCATAGGTTGATGAGAATGAAAGGGCACAACAGACAGCG
AACATGATGGGGGCACCATCGACAAGTGAGTATCAGCAGCACGGTTTTACTCCTTATGATCCAATAAGGAGCTTCCTGCAGTT
CAACATCGTGCAGCAGCCTCAGTTCTATTCTCAGCAGGAGGACCGGAAAGACTTCAACGACCAAGGTAGATTTTTTTTTTATA
TCTACCACATTTATTTGAAATGTATTTAAATGGACACCCTAAGGTATTTCATAATACCATGTATATTTTTCTAAATGAAATAA
TAATCCCTCCATCTCAAATTATTATTCATTTTAATTCTTGGTTTTTAAGAATGATGATGAATCTAGACACATACATCAATTAT
TAACTGTACGAATCTATTAGAAGTTTAAAATGAATTTTAATTTGGGACAGAGACAATATTTATTAGTCATCACAAAACTTGGA
AGCCATAGGGCTAGGAAAACAAAATCATTGCAGTTAGTTTTAAATAAAATGCCTAAACCTTCCACTTGGCTATATATATATAT
ATATATATATATATATATATATATATATATATATATATATATATATATATATATATATATATATATATATATATATATATATA
TATATATATATATATATATATATATATATATAATTTCCTCTCCAAGTACATGTTCTGCTATTCTGATTCCTTTTAATGTCTTT
AGGTGGAAGATAAATATTGGACCTCTCAAGCTTCAGTACTTATCCGTGATGATGCATGACTGCCAGTGAGAAACTGAGCTACA
TTACTGTGGAATTATATGTAAAGACTAGTACTACTGCTTCATATATGTGCATGTGCGCACACGCGCACGTAGTATGCACAATT
TCATCCCACTATTATGGCTTGGCACCAACTATGTCTCCTTAATTATCATAGGAGAAAAATAAGTCACGCACAAAAAAATTCTA
AAGATGAGGCGCAGTGTTGGATGACTGAAACTGAAAGATAGGATTCTCGTGGATGGCGGTGGTCATATATATGTCATGCACAT
CTGATCTCTGTTCCAAAATGCCACATCTTCTTTCCCAACCCACTCGAATCTACACCGAGAATATATATGTTTTATTCGTAATG
TATAGACACTATTAGCAATGATACTGTGAACTATGGTTATGGTCCACAATAAGAAATGCATATTTACCTGTG

Mapping the difference of ICS(PD₈₈) and the reference RNA sequence onto the B73 DNA that encodes the primary transcript, gives a new DNA sequence with CDS underlined and the differences highlighted in bold with wavy underline.

DNA with ZAG1 CDS increased to 50% without UTR structure constraints

SEQ ID NO: 58 provides a design for a polynucleotide sequence to help identify what needs
DNA editing with CDS trained to a 50% expression design level encoding of a
Zm00001eb284010_P001 variant of a ZAG1 protein of Zea mays
GGAGCAAGAAACACTCAGAAGCTGCCCAGAGCTACCACCCTTCTTATCCCCACCCCTCCTCCTCCTACCTTTTCTCCTTCAGA

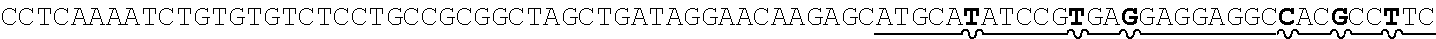

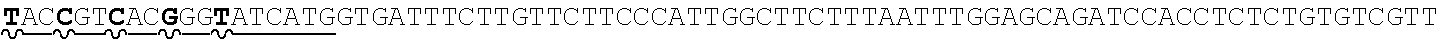

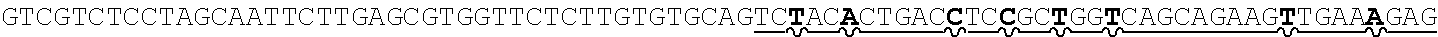

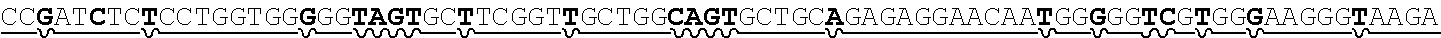

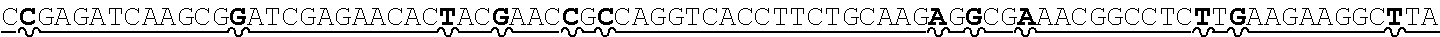

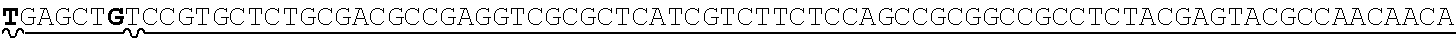

GGTCTCTTCTTTCTCTCCCCAGCTCTGTGCATCTCTCTCTCTGTGTGGTACTAGCTAGCTAGTACCGCAATATAATGGTAGTA
GGCGCTACTACGACGAATCACCTTCGCTCCAAGAAATGGTAGGCTACATATATAAGTGATGAGTTTGTCGTCCCGTCTAGCTA
GCTAGACACATGATGTGTATTTCGACGACGCTCTCCGAGAATTGAAGCAGCAAGTTAGCTCCCAGCTTTCTTGCTTGCAAAGA
GGAAAGCTGCATCTCGTTTGCTGCTACGACTACATGCGTTCTGTTTATTAACTGTCTTGCTTATTTTTTGTGCTACCGCGGAG
AGGGGCGAAAGAAATCGCAACGGTTTCTTTGCGCGCGTGCGTGCTCCGTCATCCTTTCCGGTTTCTCTTGTGTTCCTGTGAGG
TGAGCTAGATGTCTAGATAGATTCAGCGCAGGGGTATGCTTTGTGAGGTGAGCTTTTCTAGTGCGCGTGCGTGCAATGGGCGG
GTGCCTTGCGGTTGCTTTGGTTTGGTGTGGTGGTCCGCTTCTTTTCTTGCTCCATTTAATTATTTCTTTCCTTTCTATGATCT
ATATATCCCTGAGGAATATCAGTGAAGACTTATTAGATCTCTCATACCTCATGATACATAGCAAGCATTGTTCTCACACAAGG
AAAAAAAACTTTTTGTTGCTTTGGTCATGTATATATGTGCAGATCTAGTAATTATTATCCTTCGCTCTTAGATTTGGGTCACT
AGCTGGATGTGCGCGCGTGCAGTAACTCGGTAACTGGATGCAAACTTGTGAATGTGAGTGAGGGGAGGTAAGCATTTGAGACG
TAAAAAGATGCCAGGCGCGCATCCTGTCCTGCTAGTGCTATATGATGTTAGGGTTAGGGTTCCATCCCATGTGCGTCCGTCGT
CATCATCACCATCTGGCCTCTCTCTCTCTCTTCTCTCTCTCTCTCTCTCTCTCTCTCTCTCTCTCTCTCTCTCATGCTGTCAT
ACATTGTGTTTTCTGTAGATGTTACTGTTGTCCCTGCGTGTGTGGTGGTTCTGCAGGTCTCCTCGTCACAGCCATGTATATCT
GCGCGCGCGCTGTGCACTGTGCTGCATATGCATATGCTAAAGTGATGCGCAACTGCCCATCACGGGAGCTCTGTCCTCTCTGC
CACCACATAGCACTGTAGTTTTTAGGACTGGCAGGAAATAAAAGGGGGGTAATGTAATGCAGAAGGAAACGATAACATCAAAT
TGAAAACAAAGAAGCAACCTAGAAACAGCGTGATAGCAAATTAAATTTCCTTAGGCATTTGTGCTTATTTTTTAAGATTTCTT
TATCACATCCTTATGTTGCACACGTACATGCAATGCAAGTGGCATGAGTGAACGAATATATAGATTCATTACAGCTTTGACAA
ATTGGAACCACTCCATCTGATTGAATGCGTTTGACTTGTAAACTCAAAGGACAATATGTGTTGTGTTTCTTCGCAGAAATTTG
AGCATTATGATTGGGTTCTCGTATCTTCAAGTGAATATTTACTGTTAATAGTAATAGTAGTGTTCATCACACTGAACCATAAA
TTTGAGAGAAACACCATTTTGTAAGTAATCAAATTACTTTTCTTGAACAGATTCCAAAAGAATCAAAATTTCATCTATATTGT
TGCAATATTGAAACTAAAAAGTAGCTCGGATGCATTAGTCAACATGGTATTGCAAATATTTGTACAATAAAATCATAGTTTTG
AAACTGTTGTATCTATATGGTGTTTGATTGGTTACTTGTGTTTGCAATGTATTCAGGCAGTGCAGATTGGTTGTATTTGGTTT
GCCAGACATATATAGTCTCTTATTCATGCTTGCACTTTGCTCAAAGAAAGCACAATAATGCATATTAGCTAGTATGCCAGATT
TGGTCGTACTTAGACAAGGTTTGCACAATCTGATGGGATGGTCATACAAAAATAAAACAACCTCTTCCCTTTACATATACTAA
AAATATGAGAACTCAAATAGAACATGGATCGGAGGTAGCAATAAAGTGTGAAGAACCAAACATATTTTTTATACACTGCATGT
GTTTCTGATCGACTCAGGTTGCATTGCATAAGGTCTAATGCAGAGAAGACTAATGCAAGCAAACAATTAGAGCCATAACATCT
TATAATTGATATAAAAGTATACCTATCTATTTCATGATAATTTTAAATATAAAAGGTTTGCTCAAATAAAAATGCAAACTATA
ATTGATTTTTTTTTTGCTCAAAGCTCGACGGAAGCCAGATGTTCGAACGGGTGTAGCTGCAATTCATAGAGATAAAACACTGT
AGAAATATTAGAGCGACCTGTACGAATTAAAGTCAATCGACGCTATAGATATATATTTGGTCTTTGAGGGCGTACGGGGCAAC
TGAAATGTGTGCCAATTTGTTTGTGTGATGTGAATGCTCTGTATACGGAACCAGTAGCATGACCAGTTTTCCTTGCCACTGCC
AGAGTTGTATCGGGGGACTACAAGGGGAAGAAAGCTGGCAATGCAAGCTGGTGTTGGTCAGAAGGGTTATGGCACCCAACGGT
AGGGCTGACCAGTTGGGTAACAGCTGAGTCCATGTCTTATCTATGCAGCACTCCTTTGTGACCAATGGGTAGTAGATGTCCAC
TGCTTGCAGGCAATCATGTTGTGGGTAATGCATGGCTACTGCCAAGCATGCTCGAGGATGCTAAGGTTTTTGCTATGTTCACC
TACAGAGTACAGAACAGCAGTACCTTTGAAATTGGTAGCCATCCGTCTCAAAATTCGTTCTCATTTTCGAAAATGCAACCATT
TTAAACTTACAAATATATATAATAAAATATTAATATTACATAATTAGTATCGTTAGAAGATCTTTGAATCTATTTTCATAACA
AATATTTTTAAAGATACAAATATTACAAATATTTTTTTATAAATTTAAATCAGTCTTGACAAACTTTGACCTGCACGAAATGA
CTTAGACAACTATTTTAGAATGGAGAGAGCATATAAATTATGTGAGTCAAAGAGTTTCTTAGAAATAATGGTTGGCACTAAGT
GGGTTCTACATTATTGCCCCCATAAATATTTCTTTGAAGCACTGCTATTAAAATGCTTGGCCTCCGCGATGGATGTTATGAGT
TCCAAAAAAAGTGACGAGTTCCTGTTAATCTGTGTATGGCAACTGCAAAATTGGCATGTTTAGAAAATACATATCTCGTGTAA
GCATAGGGTTCAAATTTGAGTCAAGATGCATGATTCATGTCTCATATTTCTTTTAGCTAGAATAAAAGTGGTATTTACATTTC
CTTTTTGAACGTAAGACATTTACATGGATATGTCATCTTTTTCTGACAAATATTTAGACAACTGTTTTTTACCATCAAAGCTA
ATTAATGTGAATAACACATCATTTTTTAGATTAGATTGTTTAATTTTTTGCAATGAAAAAATCATTCCTTTGAAGAATCATGT
GATCCATCTTTCTCCTTTCACTTCCGTTGCACCATTAACATAAGTTATTTAACAATACAATCCATATGTTCCTTTAAATTTGT
TTATGTCCTATTTAGTAAAAATATCCACTTAAGCAACACAAAGAGAGCTGGCTCAACTTAACTGTGAATGCATCGAATCCACT
AACTATTGCGCTTTTGGTTATTATCAACAACAAATATTTTTATTAATATATCAAGATAATCTATATTTTATCAGTTTCTCAAT
GTTTCAAGATTTAAAAATATAGGTCTTGAGTTTTTTTTCTTGAGAGGGTAATTTTACCAGGATGTTTTTTGGTTCAGTTCAAA
ATTTAATTCAATCCTAATGTTTAGATCTAGACCCCCTTTTTTTAAACAGTTCTTGTAGGATTTGACTTTGTTGAAGAAAACAA
GGATGAATTTTTTTGCTATGCTAGCTTAGGAGTTCCGATTTTCTTAAAAAGAACTGTAGTAGTTATTAGGTGTGGTATTACAT
AGGTTCAAACTCCATGTATGTGCACTACAAACATATTATGGGTCTGTTTGGTTTAGCTGTAGATTCCTAAAACTCTGATTCTA
GCTATGAGCTGTGAGAAAGCTACTATGAACTGACAATTATGAGAAAACTGAAATTAATTTGGCTGAAACAACTGTCAACTATA
GATTCTATAAGAGCATGACAGCTTGTAACATAGACATCATGAATCCATAAAATCTAGGGTAGGAGGTGATTCTGAATTGAACT
AGAGGAATCTATAGTTTATCTATTCTACTATTGTATATGTTTGATTGAGATTTCAGATTTTGGACAGCAAATCTAGTTCAGAA
AGCTAAACCAAACATACCATATAAGAACTCAAGTTCTATGTAGATGCATCAGTATTTATTAGAATGCTAACTCAATGGCAGTT
AGGATTTGATTATGCTGGCAATGCGATAGTATACAATATTAGACTATTAGTGAAGAGTGAAGCAGTAATGTAAGCAATGCATG
TGCTTACTAAAACGTGATAAATCATTGTTGCACCGAATATTAATACACAACATTAGGATGTGCTTGCACTTCCTGTTATTATC
TGTAGCATTGACGAGGACCACTCTCTATTTTCACATGACACTAAATAATGTGTTAAAATCAACTACTTCTAAGTGATTGCAGC
AGTATTTACCACACCAAATTAATATCACAAAATAGATTATGAAATATATTTTCATAATTTAACCATTTAGAGTCCTAGATGAT
GTTACTATTTAAGTGATTGCTGGGATGGCTTTACTTTAGCATGGAAGTATTAATAGTATTTTGGGACGATGCGTTAGAATATT
GGTTACTTTATGTGCTGATTGTATTGACTTTCACTAATGGGGCTGTTCTGGCTAGATTATTAAAGCCGTTCGGACTGCTGCTG
CCGTATAGACAAAATACTGTAGAAAAGGTAGAAGCCGGTGGATTAAAGCCGAAATACTGTAGAAAAAGTAGAAGCCGGTGGAT
TAAAGCCGCAACGGACAGGATGAATGTCAAATTGTAATCGACATACTTGCTAGAAGGCAATGTATCCATTATAATCGCTGGAA
GATGTCAAATTTCAGTTATTGTATATGTTCTATGAATCTCCATTATAATTTATTATGCTTGTGACGATTGTTCTATTATCTGT
TGTATTTTTTCAAGAAAAATATCTAGTTTTGCCCTGGAACAATTTTGTTTCATATTTTCATTCTTTCGGCACATACAATTATC
TCACCTGATTGAATGTATGTGTACTAACTACTAACTACAGTATTATATTGTATGTACAGCGTGAAGGGCACCATTGAGAGGTA
CAAGAAGGCAACCAGTGACAACTCCAGCGCAGCTGGTACGATTGCAGAGGTCACCATTCAGGTAACATCATGTCGATCAGTCA
ACACATCGTTTCTGATCCTACAAGACCCTAGTCGTAGTGTTGGCTACCACAGGCTATGCTGTCTATTGATGACCCTAGTGTTG
TACTATGCAACATTCCTTGCAACTGTGTGCTCTACACAGCCAAAATATTTTCTCTGGGCGAAATCTTCAGACTGGACATGCAC
CCTGTGGGTTCTGGGCATTACACGGCAGAAATTCCAGTGACGATACTAAGAGAACCTGCTTGGACCAGCATTTCATGTGGAGT
TGTGCAGATATATAATTGTCCTCTCTGGATGTCTGGGTAGCTTTGGTTATTCCATGCTCCAAATGGTAGAGAGGATAGCATGC
ATGTTTTGGACAAAAACGAATAAAACCACTGAAGGCTAGTAGGAAAATTCTTCTTGATGGGTTTTCGTAAGATGTGGCAATTA
TAATATTCCTCTGATAAATATATATATGGCTAGTAGGAAACTTTATAAGTATATATATGGCAGTCAGTTTGGTGAAATGGAGT
TTTAGTGTTGCACATATTTTCTTTTCCCTGAAATTGATTTCGATGAATAAATTAATTGGACTACTTTTGTTTTTTCTTGCAAT
TGAGAAATGAGTACACCATTTAATTCACATTTTGCAGTCGGACCTGTTTGTTATGAATGTCAATATGGAACATCCACTAACAA
TTTGGATAGAAACCAACAGCAGCATGAAATATTACTAAATGAACTTTTGCAAACTTGCAGCATTACAAGCAGGAATCTGCTAG
GCTGAGGCAGCAGATCGTTAACTTGCAGAACTCCAACAGGTTCAGTTCTTATAGAAAATTATCATGTGCGCGAGAATACAGAA
AGGTCATTTGATGAACTTATAAGTTTTCAGGGCCCTGATAGGTGATTCTATCACAACCATGAGCCACAAGGAACTTAAGCACT
TGGAGACTAGGTTAGACAAAGCTCTCGGAAAGATTAGAGCAAAAAAGGTACTACGTGCTATATATCTATAATAATCATATTTA
CTGCATTATGTGTGGAAAAATAAATATAGTATTTCCTTTTACGCAATTAATGCGGATCAAACTCTGCAGAACGATGTGCTGTG
TTCTGAAGTCGAGTACATGCAGAGAAGGGTAAACAGCAACAAGAACACATTTTTCCTCTCACGAACTTATTGATTTACTCGAC
ATTGAACAAAATAAAAACAAGAATTTCGCATTTTCAGGAAATGGAGTTGCAGAATGACAACTTGTACTTAAGGAGCCGGGTAA
GTAACCTGTAGTTGCTAATTAAGAAGGACAGTCTTAGAAGCAAAATATGGAACTGTAATTACTAAAATGCCAAGTTTACCTTA
ACTTTGAAAATAAAAAATAGTGTAACAATTTAACTGTCTCACAATGCAAGCATAGGTTGATGAGAATGAAAGGGCACAACAGA
CAGCGAACATGATGGGGGCACCATCGACAAGTGAGTATCAGCAGCACGGTTTTACTCCTTATGATCCAATAAGGAGCTTCCTG
CAGTTCAACATCGTGCAGCAGCCTCAGTTCTATTCTCAGCAGGAGGACCGGAAAGACTTCAACGACCAAGGTAGATTTTTTTT
TTATATCTACCACATTTATTTGAAATGTATTTAAATGGACACCCTAAGGTATTTCATAATACCATGTATATTTTTCTAAATGA
AATAATAATCCCTCCATCTCAAATTATTATTCATTTTAATTCTTGGTTTTTAAGAATGATGATGAATCTAGACACATACATCA
ATTATTAACTGTACGAATCTATTAGAAGTTTAAAATGAATTTTAATTTGGGACAGAGACAATATTTATTAGTCATCACAAAAC
TTGGAAGCCATAGGGCTAGGAAAACAAAATCATTGCAGTTAGTTTTAAATAAAATGCCTAAACCTTCCACTTGGCTATATATA
TATATATATATATATATATATATATATATATATATATATATATATATATATATATATATATATATATATATATATATATATAT
ATATATATATATATATATATATATATATATATATATAATTTCCTCTCCAAGTACATGTTCTGCTATTCTGATTCCTTTTAATG
TCTTTAGGTGGAAGATAAATATTGGACCTCTCAAGCTTCAGTACTTATCCGTGATGATGCATGACTGCCAGTGAGAAACTGAG
CTACATTACTGTGGAATTATATGTAAAGACTAGTACTACTGCTTCATATATGTGCATGTGCGCACACGCGCACGTAGTATGCA
CAATTTCATCCCACTATTATGGCTTGGCACCAACTATGTCTCCTTAATTATCATAGGAGAAAAATAAGTCACGCACAAAAAAA
TTCTAAAGATGAGGCGCAGTGTTGGATGACTGAAACTGAAAGATAGGATTCTCGTGGATGGCGGTGGTCATATATATGTCATG
CACATCTGATCTCTGTTCCAAAATGCCACATCTTCTTTCCCAACCCACTCGAATCTACACCGAGAATATATATGTTTTATTCG
TAATGTATAGACACTATTAGCAATGATACTGTGAACTATGGTTATGGTCCACAATAAGAAATGCATATTTACCTGTG

DNA with ZAG1 CDS decreased to 30% without UTR structure constraints, coding regions underlined and differences from reference in bold with wavy underline

SEQ ID NO: 59 provides a design for a polynucleotide sequence to help identify what needs
DNA editing with CDS trained to a 30% expression design level with putative encoding of a
Zm00001eb284010_P001 variant of a ZAG1 protein of Zea mays
GGAGCAAGAAACACTCAGAAGCTGCCCAGAGCTACCACCCTTCTTATCCCCACCCCTCCTCCTCCTACCTTTTCTCCTTCAGA

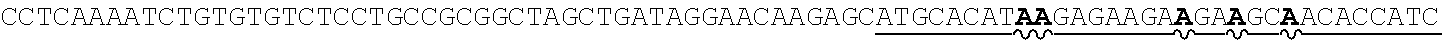

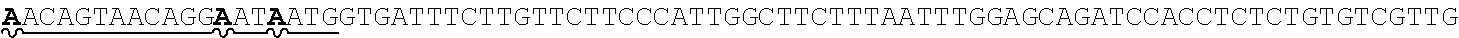

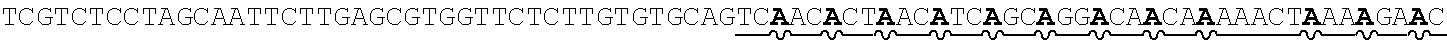

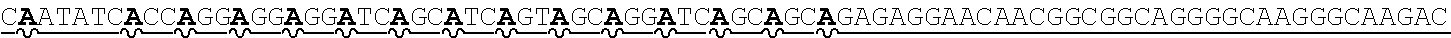

TGAGATCAAGCGCATCGAGAACACGACCAACAGGCAGGTCACCTTCTGCAAGCGCCGCAACGGCCTCCTCAAGAAGGCGTACG
AGCTCTCCGTGCTCTGCGACGCCGAGGTCGCGCTCATCGTCTTCTCCAGCCGCGGCCGCCTCTACGAGTACGCCAACAACAGG
TCTCTTCTTTCTCTCCCCAGCTCTGTGCATCTCTCTCTCTGTGTGGTACTAGCTAGCTAGTACCGCAATATAATGGTAGTAGG
CGCTACTACGACGAATCACCTTCGCTCCAAGAAATGGTAGGCTACATATATAAGTGATGAGTTTGTCGTCCCGTCTAGCTAGC
TAGACACATGATGTGTATTTCGACGACGCTCTCCGAGAATTGAAGCAGCAAGTTAGCTCCCAGCTTTCTTGCTTGCAAAGAGG
AAAGCTGCATCTCGTTTGCTGCTACGACTACATGCGTTCTGTTTATTAACTGTCTTGCTTATTTTTTGTGCTACCGCGGAGAG
GGGCGAAAGAAATCGCAACGGTTTCTTTGCGCGCGTGCGTGCTCCGTCATCCTTTCCGGTTTCTCTTGTGTTCCTGTGAGGTG
AGCTAGATGTCTAGATAGATTCAGCGCAGGGGTATGCTTTGTGAGGTGAGCTTTTCTAGTGCGCGTGCGTGCAATGGGCGGGT
GCCTTGCGGTTGCTTTGGTTTGGTGTGGTGGTCCGCTTCTTTTCTTGCTCCATTTAATTATTTCTTTCCTTTCTATGATCTAT
ATATCCCTGAGGAATATCAGTGAAGACTTATTAGATCTCTCATACCTCATGATACATAGCAAGCATTGTTCTCACACAAGGAA
AAAAAACTTTTTGTTGCTTTGGTCATGTATATATGTGCAGATCTAGTAATTATTATCCTTCGCTCTTAGATTTGGGTCACTAG
CTGGATGTGCGCGCGTGCAGTAACTCGGTAACTGGATGCAAACTTGTGAATGTGAGTGAGGGGAGGTAAGCATTTGAGACGTA
AAAAGATGCCAGGCGCGCATCCTGTCCTGCTAGTGCTATATGATGTTAGGGTTAGGGTTCCATCCCATGTGCGTCCGTCGTCA
TCATCACCATCTGGCCTCTCTCTCTCTCTTCTCTCTCTCTCTCTCTCTCTCTCTCTCTCTCTCTCTCTCTCATGCTGTCATAC
ATTGTGTTTTCTGTAGATGTTACTGTTGTCCCTGCGTGTGTGGTGGTTCTGCAGGTCTCCTCGTCACAGCCATGTATATCTGC
GCGCGCGCTGTGCACTGTGCTGCATATGCATATGCTAAAGTGATGCGCAACTGCCCATCACGGGAGCTCTGTCCTCTCTGCCA
CCACATAGCACTGTAGTTTTTAGGACTGGCAGGAAATAAAAGGGGGGTAATGTAATGCAGAAGGAAACGATAACATCAAATTG
AAAACAAAGAAGCAACCTAGAAACAGCGTGATAGCAAATTAAATTTCCTTAGGCATTTGTGCTTATTTTTTAAGATTTCTTTA
TCACATCCTTATGTTGCACACGTACATGCAATGCAAGTGGCATGAGTGAACGAATATATAGATTCATTACAGCTTTGACAAAT
TGGAACCACTCCATCTGATTGAATGCGTTTGACTTGTAAACTCAAAGGACAATATGTGTTGTGTTTCTTCGCAGAAATTTGAG
CATTATGATTGGGTTCTCGTATCTTCAAGTGAATATTTACTGTTAATAGTAATAGTAGTGTTCATCACACTGAACCATAAATT
TGAGAGAAACACCATTTTGTAAGTAATCAAATTACTTTTCTTGAACAGATTCCAAAAGAATCAAAATTTCATCTATATTGTTG
CAATATTGAAACTAAAAAGTAGCTCGGATGCATTAGTCAACATGGTATTGCAAATATTTGTACAATAAAATCATAGTTTTGAA
ACTGTTGTATCTATATGGTGTTTGATTGGTTACTTGTGTTTGCAATGTATTCAGGCAGTGCAGATTGGTTGTATTTGGTTTGC
CAGACATATATAGTCTCTTATTCATGCTTGCACTTTGCTCAAAGAAAGCACAATAATGCATATTAGCTAGTATGCCAGATTTG
GTCGTACTTAGACAAGGTTTGCACAATCTGATGGGATGGTCATACAAAAATAAAACAACCTCTTCCCTTTACATATACTAAAA
ATATGAGAACTCAAATAGAACATGGATCGGAGGTAGCAATAAAGTGTGAAGAACCAAACATATTTTTTATACACTGCATGTGT
TTCTGATCGACTCAGGTTGCATTGCATAAGGTCTAATGCAGAGAAGACTAATGCAAGCAAACAATTAGAGCCATAACATCTTA
TAATTGATATAAAAGTATACCTATCTATTTCATGATAATTTTAAATATAAAAGGTTTGCTCAAATAAAAATGCAAACTATAAT
TGATTTTTTTTTTGCTCAAAGCTCGACGGAAGCCAGATGTTCGAACGGGTGTAGCTGCAATTCATAGAGATAAAACACTGTAG
AAATATTAGAGCGACCTGTACGAATTAAAGTCAATCGACGCTATAGATATATATTTGGTCTTTGAGGGCGTACGGGGCAACTG
AAATGTGTGCCAATTTGTTTGTGTGATGTGAATGCTCTGTATACGGAACCAGTAGCATGACCAGTTTTCCTTGCCACTGCCAG
AGTTGTATCGGGGGACTACAAGGGGAAGAAAGCTGGCAATGCAAGCTGGTGTTGGTCAGAAGGGTTATGGCACCCAACGGTAG
GGCTGACCAGTTGGGTAACAGCTGAGTCCATGTCTTATCTATGCAGCACTCCTTTGTGACCAATGGGTAGTAGATGTCCACTG
CTTGCAGGCAATCATGTTGTGGGTAATGCATGGCTACTGCCAAGCATGCTCGAGGATGCTAAGGTTTTTGCTATGTTCACCTA
CAGAGTACAGAACAGCAGTACCTTTGAAATTGGTAGCCATCCGTCTCAAAATTCGTTCTCATTTTCGAAAATGCAACCATTTT
AAACTTACAAATATATATAATAAAATATTAATATTACATAATTAGTATCGTTAGAAGATCTTTGAATCTATTTTCATAACAAA
TATTTTTAAAGATACAAATATTACAAATATTTTTTTATAAATTTAAATCAGTCTTGACAAACTTTGACCTGCACGAAATGACT
TAGACAACTATTTTAGAATGGAGAGAGCATATAAATTATGTGAGTCAAAGAGTTTCTTAGAAATAATGGTTGGCACTAAGTGG
GTTCTACATTATTGCCCCCATAAATATTTCTTTGAAGCACTGCTATTAAAATGCTTGGCCTCCGCGATGGATGTTATGAGTTC
CAAAAAAAGTGACGAGTTCCTGTTAATCTGTGTATGGCAACTGCAAAATTGGCATGTTTAGAAAATACATATCTCGTGTAAGC
ATAGGGTTCAAATTTGAGTCAAGATGCATGATTCATGTCTCATATTTCTTTTAGCTAGAATAAAAGTGGTATTTACATTTCCT
TTTTGAACGTAAGACATTTACATGGATATGTCATCTTTTTCTGACAAATATTTAGACAACTGTTTTTTACCATCAAAGCTAAT
TAATGTGAATAACACATCATTTTTTAGATTAGATTGTTTAATTTTTTGCAATGAAAAAATCATTCCTTTGAAGAATCATGTGA
TCCATCTTTCTCCTTTCACTTCCGTTGCACCATTAACATAAGTTATTTAACAATACAATCCATATGTTCCTTTAAATTTGTTT
ATGTCCTATTTAGTAAAAATATCCACTTAAGCAACACAAAGAGAGCTGGCTCAACTTAACTGTGAATGCATCGAATCCACTAA
CTATTGCGCTTTTGGTTATTATCAACAACAAATATTTTTATTAATATATCAAGATAATCTATATTTTATCAGTTTCTCAATGT
TTCAAGATTTAAAAATATAGGTCTTGAGTTTTTTTTCTTGAGAGGGTAATTTTACCAGGATGTTTTTTGGTTCAGTTCAAAAT
TTAATTCAATCCTAATGTTTAGATCTAGACCCCCTTTTTTTAAACAGTTCTTGTAGGATTTGACTTTGTTGAAGAAAACAAGG
ATGAATTTTTTTGCTATGCTAGCTTAGGAGTTCCGATTTTCTTAAAAAGAACTGTAGTAGTTATTAGGTGTGGTATTACATAG
GTTCAAACTCCATGTATGTGCACTACAAACATATTATGGGTCTGTTTGGTTTAGCTGTAGATTCCTAAAACTCTGATTCTAGC
TATGAGCTGTGAGAAAGCTACTATGAACTGACAATTATGAGAAAACTGAAATTAATTTGGCTGAAACAACTGTCAACTATAGA
TTCTATAAGAGCATGACAGCTTGTAACATAGACATCATGAATCCATAAAATCTAGGGTAGGAGGTGATTCTGAATTGAACTAG
AGGAATCTATAGTTTATCTATTCTACTATTGTATATGTTTGATTGAGATTTCAGATTTTGGACAGCAAATCTAGTTCAGAAAG
CTAAACCAAACATACCATATAAGAACTCAAGTTCTATGTAGATGCATCAGTATTTATTAGAATGCTAACTCAATGGCAGTTAG
GATTTGATTATGCTGGCAATGCGATAGTATACAATATTAGACTATTAGTGAAGAGTGAAGCAGTAATGTAAGCAATGCATGTG
CTTACTAAAACGTGATAAATCATTGTTGCACCGAATATTAATACACAACATTAGGATGTGCTTGCACTTCCTGTTATTATCTG
TAGCATTGACGAGGACCACTCTCTATTTTCACATGACACTAAATAATGTGTTAAAATCAACTACTTCTAAGTGATTGCAGCAG
TATTTACCACACCAAATTAATATCACAAAATAGATTATGAAATATATTTTCATAATTTAACCATTTAGAGTCCTAGATGATGT
TACTATTTAAGTGATTGCTGGGATGGCTTTACTTTAGCATGGAAGTATTAATAGTATTTTGGGACGATGCGTTAGAATATTGG
TTACTTTATGTGCTGATTGTATTGACTTTCACTAATGGGGCTGTTCTGGCTAGATTATTAAAGCCGTTCGGACTGCTGCTGCC
GTATAGACAAAATACTGTAGAAAAGGTAGAAGCCGGTGGATTAAAGCCGAAATACTGTAGAAAAAGTAGAAGCCGGTGGATTA
AAGCCGCAACGGACAGGATGAATGTCAAATTGTAATCGACATACTTGCTAGAAGGCAATGTATCCATTATAATCGCTGGAAGA
TGTCAAATTTCAGTTATTGTATATGTTCTATGAATCTCCATTATAATTTATTATGCTTGTGACGATTGTTCTATTATCTGTTG
TATTTTTTCAAGAAAAATATCTAGTTTTGCCCTGGAACAATTTTGTTTCATATTTTCATTCTTTCGGCACATACAATTATCTC
ACCTGATTGAATGTATGTGTACTAACTACTAACTACAGTATTATATTGTATGTACAGCGTGAAGGGCACCATTGAGAGGTACA
AGAAGGCAACCAGTGACAACTCCAGCGCAGCTGGTACGATTGCAGAGGTCACCATTCAGGTAACATCATGTCGATCAGTCAAC
ACATCGTTTCTGATCCTACAAGACCCTAGTCGTAGTGTTGGCTACCACAGGCTATGCTGTCTATTGATGACCCTAGTGTTGTA
CTATGCAACATTCCTTGCAACTGTGTGCTCTACACAGCCAAAATATTTTCTCTGGGCGAAATCTTCAGACTGGACATGCACCC
TGTGGGTTCTGGGCATTACACGGCAGAAATTCCAGTGACGATACTAAGAGAACCTGCTTGGACCAGCATTTCATGTGGAGTTG
TGCAGATATATAATTGTCCTCTCTGGATGTCTGGGTAGCTTTGGTTATTCCATGCTCCAAATGGTAGAGAGGATAGCATGCAT
GTTTTGGACAAAAACGAATAAAACCACTGAAGGCTAGTAGGAAAATTCTTCTTGATGGGTTTTCGTAAGATGTGGCAATTATA
ATATTCCTCTGATAAATATATATATGGCTAGTAGGAAACTTTATAAGTATATATATGGCAGTCAGTTTGGTGAAATGGAGTTT
TAGTGTTGCACATATTTTCTTTTCCCTGAAATTGATTTCGATGAATAAATTAATTGGACTACTTTTGTTTTTTCTTGCAATTG
AGAAATGAGTACACCATTTAATTCACATTTTGCAGTCGGACCTGTTTGTTATGAATGTCAATATGGAACATCCACTAACAATT
TGGATAGAAACCAACAGCAGCATGAAATATTACTAAATGAACTTTTGCAAACTTGCAGCATTACAAGCAGGAATCTGCTAGGC
TGAGGCAGCAGATCGTTAACTTGCAGAACTCCAACAGGTTCAGTTCTTATAGAAAATTATCATGTGCGCGAGAATACAGAAAG
GTCATTTGATGAACTTATAAGTTTTCAGGGCCCTGATAGGTGATTCTATCACAACCATGAGCCACAAGGAACTTAAGCACTTG
GAGACTAGGTTAGACAAAGCTCTCGGAAAGATTAGAGCAAAAAAGGTACTACGTGCTATATATCTATAATAATCATATTTACT
GCATTATGTGTGGAAAAATAAATATAGTATTTCCTTTTACGCAATTAATGCGGATCAAACTCTGCAGAACGATGTGCTGTGTT
CTGAAGTCGAGTACATGCAGAGAAGGGTAAACAGCAACAAGAACACATTTTTCCTCTCACGAACTTATTGATTTACTCGACAT
TGAACAAAATAAAAACAAGAATTTCGCATTTTCAGGAAATGGAGTTGCAGAATGACAACTTGTACTTAAGGAGCCGGGTAAGT
AACCTGTAGTTGCTAATTAAGAAGGACAGTCTTAGAAGCAAAATATGGAACTGTAATTACTAAAATGCCAAGTTTACCTTAAC
TTTGAAAATAAAAAATAGTGTAACAATTTAACTGTCTCACAATGCAAGCATAGGTTGATGAGAATGAAAGGGCACAACAGACA
GCGAACATGATGGGGGCACCATCGACAAGTGAGTATCAGCAGCACGGTTTTACTCCTTATGATCCAATAAGGAGCTTCCTGCA
GTTCAACATCGTGCAGCAGCCTCAGTTCTATTCTCAGCAGGAGGACCGGAAAGACTTCAACGACCAAGGTAGATTTTTTTTTT
ATATCTACCACATTTATTTGAAATGTATTTAAATGGACACCCTAAGGTATTTCATAATACCATGTATATTTTTCTAAATGAAA
TAATAATCCCTCCATCTCAAATTATTATTCATTTTAATTCTTGGTTTTTAAGAATGATGATGAATCTAGACACATACATCAAT
TATTAACTGTACGAATCTATTAGAAGTTTAAAATGAATTTTAATTTGGGACAGAGACAATATTTATTAGTCATCACAAAACTT
GGAAGCCATAGGGCTAGGAAAACAAAATCATTGCAGTTAGTTTTAAATAAAATGCCTAAACCTTCCACTTGGCTATATATATA
TATATATATATATATATATATATATATATATATATATATATATATATATATATATATATATATATATATATATATATATATAT
ATATATATATATATATATATATATATATATATATAATTTCCTCTCCAAGTACATGTTCTGCTATTCTGATTCCTTTTAATGTC
TTTAGGTGGAAGATAAATATTGGACCTCTCAAGCTTCAGTACTTATCCGTGATGATGCATGACTGCCAGTGAGAAACTGAGCT
ACATTACTGTGGAATTATATGTAAAGACTAGTACTACTGCTTCATATATGTGCATGTGCGCACACGCGCACGTAGTATGCACA
ATTTCATCCCACTATTATGGCTTGGCACCAACTATGTCTCCTTAATTATCATAGGAGAAAAATAAGTCACGCACAAAAAAATT
CTAAAGATGAGGCGCAGTGTTGGATGACTGAAACTGAAAGATAGGATTCTCGTGGATGGCGGTGGTCATATATATGTCATGCA
CATCTGATCTCTGTTCCAAAATGCCACATCTTCTTTCCCAACCCACTCGAATCTACACCGAGAATATATATGTTTTATTCGTA
ATGTATAGACACTATTAGCAATGATACTGTGAACTATGGTTATGGTCCACAATAAGAAATGCATATTTACCTGTG

DNA with ZAG1 CDS increased to 50% with UTR structure constraints, coding region underlined and differences from reference in bold with wavy underline,

SEQ ID NO: 60 provides a design for a polynucleotide sequence to help identify what needs
DNA editing with CDS trained with Zm00001eb284010_T001 UTR-sequence-and-structure
constraints to a 50% expression design level with putative encoding of a
Zm00001eb284010_P001 variant of a ZAG1 protein of Zea mays
GGAGCAAGAAACACTCAGAAGCTGCCCAGAGCTACCACCCTTCTTATCCCCACCCCTCCTCCTCCTACCTTTTCTCCTTCAGA

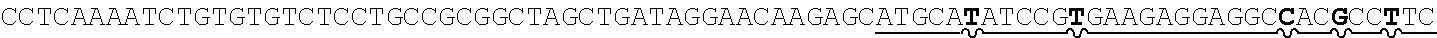

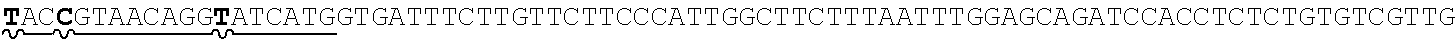

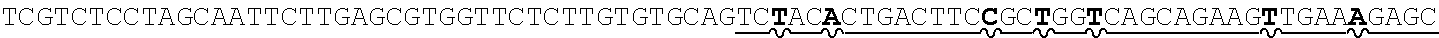

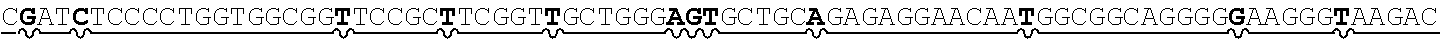

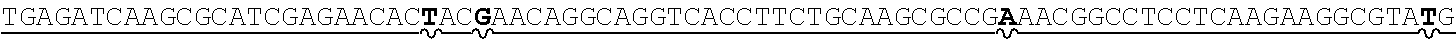

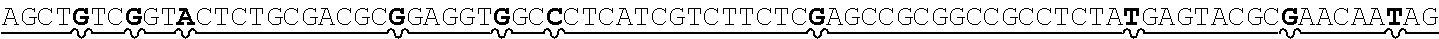

GTCTCTTCTTTCTCTCCCCAGCTCTGTGCATCTCTCTCTCTGTGTGGTACTAGCTAGCTAGTACCGCAATATAATGGTAGTAG
GCGCTACTACGACGAATCACCTTCGCTCCAAGAAATGGTAGGCTACATATATAAGTGATGAGTTTGTCGTCCCGTCTAGCTAG
CTAGACACATGATGTGTATTTCGACGACGCTCTCCGAGAATTGAAGCAGCAAGTTAGCTCCCAGCTTTCTTGCTTGCAAAGAG
GAAAGCTGCATCTCGTTTGCTGCTACGACTACATGCGTTCTGTTTATTAACTGTCTTGCTTATTTTTTGTGCTACCGCGGAGA
GGGGCGAAAGAAATCGCAACGGTTTCTTTGCGCGCGTGCGTGCTCCGTCATCCTTTCCGGTTTCTCTTGTGTTCCTGTGAGGT
GAGCTAGATGTCTAGATAGATTCAGCGCAGGGGTATGCTTTGTGAGGTGAGCTTTTCTAGTGCGCGTGCGTGCAATGGGGGG
TGCCTTGCGGTTGCTTTGGTTTGGTGTGGTGGTCCGCTTCTTTTCTTGCTCCATTTAATTATTTCTTTCCTTTCTATGATCTA
TATATCCCTGAGGAATATCAGTGAAGACTTATTAGATCTCTCATACCTCATGATACATAGCAAGCATTGTTCTCACACAAGGA
AAAAAAACTTTTTGTTGCTTTGGTCATGTATATATGTGCAGATCTAGTAATTATTATCCTTCGCTCTTAGATTTGGGTCACTA
GCTGGATGTGCGCGCGTGCAGTAACTCGGTAACTGGATGCAAACTTGTGAATGTGAGTGAGGGGAGGTAAGCATTTGAGACGT
AAAAAGATGCCAGGCGCGCATCCTGTCCTGCTAGTGCTATATGATGTTAGGGTTAGGGTTCCATCCCATGTGCGTCCGTCGTC
ATCATCACCATCTGGCCTCTCTCTCTCTCTTCTCTCTCTCTCTCTCTCTCTCTCTCTCTCTCTCTCTCTCTCATGCTGTCATA
CATTGTGTTTTCTGTAGATGTTACTGTTGTCCCTGCGTGTGTGGTGGTTCTGCAGGTCTCCTCGTCACAGCCATGTATATCTG
CGCGCGCGCTGTGCACTGTGCTGCATATGCATATGCTAAAGTGATGCGCAACTGCCCATCACGGGAGCTCTGTCCTCTCTGCC
ACCACATAGCACTGTAGTTTTTAGGACTGGCAGGAAATAAAAGGGGGGTAATGTAATGCAGAAGGAAACGATAACATCAAATT
GAAAACAAAGAAGCAACCTAGAAACAGCGTGATAGCAAATTAAATTTCCTTAGGCATTTGTGCTTATTTTTTAAGATTTCTTT
ATCACATCCTTATGTTGCACACGTACATGCAATGCAAGTGGCATGAGTGAACGAATATATAGATTCATTACAGCTTTGACAAA
TTGGAACCACTCCATCTGATTGAATGCGTTTGACTTGTAAACTCAAAGGACAATATGTGTTGTGTTTCTTCGCAGAAATTTGA
GCATTATGATTGGGTTCTCGTATCTTCAAGTGAATATTTACTGTTAATAGTAATAGTAGTGTTCATCACACTGAACCATAAAT
TTGAGAGAAACACCATTTTGTAAGTAATCAAATTACTTTTCTTGAACAGATTCCAAAAGAATCAAAATTTCATCTATATTGTT
GCAATATTGAAACTAAAAAGTAGCTCGGATGCATTAGTCAACATGGTATTGCAAATATTTGTACAATAAAATCATAGTTTTGA
AACTGTTGTATCTATATGGTGTTTGATTGGTTACTTGTGTTTGCAATGTATTCAGGCAGTGCAGATTGGTTGTATTTGGTTTG
CCAGACATATATAGTCTCTTATTCATGCTTGCACTTTGCTCAAAGAAAGCACAATAATGCATATTAGCTAGTATGCCAGATTT
GGTCGTACTTAGACAAGGTTTGCACAATCTGATGGGATGGTCATACAAAAATAAAACAACCTCTTCCCTTTACATATACTAAA
AATATGAGAACTCAAATAGAACATGGATCGGAGGTAGCAATAAAGTGTGAAGAACCAAACATATTTTTTATACACTGCATGTG
TTTCTGATCGACTCAGGTTGCATTGCATAAGGTCTAATGCAGAGAAGACTAATGCAAGCAAACAATTAGAGCCATAACATCTT
ATAATTGATATAAAAGTATACCTATCTATTTCATGATAATTTTAAATATAAAAGGTTTGCTCAAATAAAAATGCAAACTATAA
TTGATTTTTTTTTTGCTCAAAGCTCGACGGAAGCCAGATGTTCGAACGGGTGTAGCTGCAATTCATAGAGATAAAACACTGTA
GAAATATTAGAGCGACCTGTACGAATTAAAGTCAATCGACGCTATAGATATATATTTGGTCTTTGAGGGCGTACGGGGCAACT
GAAATGTGTGCCAATTTGTTTGTGTGATGTGAATGCTCTGTATACGGAACCAGTAGCATGACCAGTTTTCCTTGCCACTGCCA
GAGTTGTATCGGGGGACTACAAGGGGAAGAAAGCTGGCAATGCAAGCTGGTGTTGGTCAGAAGGGTTATGGCACCCAACGGTA
GGGCTGACCAGTTGGGTAACAGCTGAGTCCATGTCTTATCTATGCAGCACTCCTTTGTGACCAATGGGTAGTAGATGTCCACT
GCTTGCAGGCAATCATGTTGTGGGTAATGCATGGCTACTGCCAAGCATGCTCGAGGATGCTAAGGTTTTTGCTATGTTCACCT
ACAGAGTACAGAACAGCAGTACCTTTGAAATTGGTAGCCATCCGTCTCAAAATTCGTTCTCATTTTCGAAAATGCAACCATTT
TAAACTTACAAATATATATAATAAAATATTAATATTACATAATTAGTATCGTTAGAAGATCTTTGAATCTATTTTCATAACAA
ATATTTTTAAAGATACAAATATTACAAATATTTTTTTATAAATTTAAATCAGTCTTGACAAACTTTGACCTGCACGAAATGAC
TTAGACAACTATTTTAGAATGGAGAGAGCATATAAATTATGTGAGTCAAAGAGTTTCTTAGAAATAATGGTTGGCACTAAGTG
GGTTCTACATTATTGCCCCCATAAATATTTCTTTGAAGCACTGCTATTAAAATGCTTGGCCTCCGCGATGGATGTTATGAGTT
CCAAAAAAAGTGACGAGTTCCTGTTAATCTGTGTATGGCAACTGCAAAATTGGCATGTTTAGAAAATACATATCTCGTGTAAG
CATAGGGTTCAAATTTGAGTCAAGATGCATGATTCATGTCTCATATTTCTTTTAGCTAGAATAAAAGTGGTATTTACATTTCC
TTTTTGAACGTAAGACATTTACATGGATATGTCATCTTTTTCTGACAAATATTTAGACAACTGTTTTTTACCATCAAAGCTAA
TTAATGTGAATAACACATCATTTTTTAGATTAGATTGTTTAATTTTTTGCAATGAAAAAATCATTCCTTTGAAGAATCATGTG
ATCCATCTTTCTCCTTTCACTTCCGTTGCACCATTAACATAAGTTATTTAACAATACAATCCATATGTTCCTTTAAATTTGTT
TATGTCCTATTTAGTAAAAATATCCACTTAAGCAACACAAAGAGAGCTGGCTCAACTTAACTGTGAATGCATCGAATCCACTA
ACTATTGCGCTTTTGGTTATTATCAACAACAAATATTTTTATTAATATATCAAGATAATCTATATTTTATCAGTTTCTCAATG
TTTCAAGATTTAAAAATATAGGTCTTGAGTTTTTTTTCTTGAGAGGGTAATTTTACCAGGATGTTTTTTGGTTCAGTTCAAAA
TTTAATTCAATCCTAATGTTTAGATCTAGACCCCCTTTTTTTAAACAGTTCTTGTAGGATTTGACTTTGTTGAAGAAAACAAG
GATGAATTTTTTTGCTATGCTAGCTTAGGAGTTCCGATTTTCTTAAAAAGAACTGTAGTAGTTATTAGGTGTGGTATTACATA
GGTTCAAACTCCATGTATGTGCACTACAAACATATTATGGGTCTGTTTGGTTTAGCTGTAGATTCCTAAAACTCTGATTCTAG
CTATGAGCTGTGAGAAAGCTACTATGAACTGACAATTATGAGAAAACTGAAATTAATTTGGCTGAAACAACTGTCAACTATAG
ATTCTATAAGAGCATGACAGCTTGTAACATAGACATCATGAATCCATAAAATCTAGGGTAGGAGGTGATTCTGAATTGAACTA
GAGGAATCTATAGTTTATCTATTCTACTATTGTATATGTTTGATTGAGATTTCAGATTTTGGACAGCAAATCTAGTTCAGAAA
GCTAAACCAAACATACCATATAAGAACTCAAGTTCTATGTAGATGCATCAGTATTTATTAGAATGCTAACTCAATGGCAGTTA
GGATTTGATTATGCTGGCAATGCGATAGTATACAATATTAGACTATTAGTGAAGAGTGAAGCAGTAATGTAAGCAATGCATGT
GCTTACTAAAACGTGATAAATCATTGTTGCACCGAATATTAATACACAACATTAGGATGTGCTTGCACTTCCTGTTATTATCT
GTAGCATTGACGAGGACCACTCTCTATTTTCACATGACACTAAATAATGTGTTAAAATCAACTACTTCTAAGTGATTGCAGCA
GTATTTACCACACCAAATTAATATCACAAAATAGATTATGAAATATATTTTCATAATTTAACCATTTAGAGTCCTAGATGATG
TTACTATTTAAGTGATTGCTGGGATGGCTTTACTTTAGCATGGAAGTATTAATAGTATTTTGGGACGATGCGTTAGAATATTG
GTTACTTTATGTGCTGATTGTATTGACTTTCACTAATGGGGCTGTTCTGGCTAGATTATTAAAGCCGTTCGGACTGCTGCTGC
CGTATAGACAAAATACTGTAGAAAAGGTAGAAGCCGGTGGATTAAAGCCGAAATACTGTAGAAAAAGTAGAAGCCGGTGGATT
AAAGCCGCAACGGACAGGATGAATGTCAAATTGTAATCGACATACTTGCTAGAAGGCAATGTATCCATTATAATCGCTGGAAG
ATGTCAAATTTCAGTTATTGTATATGTTCTATGAATCTCCATTATAATTTATTATGCTTGTGACGATTGTTCTATTATCTGTT
GTATTTTTTCAAGAAAAATATCTAGTTTTGCCCTGGAACAATTTTGTTTCATATTTTCATTCTTTCGGCACATACAATTATCT

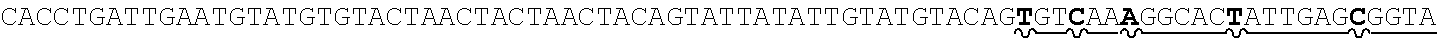

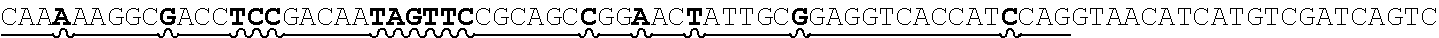

AACACATCGTTTCTGATCCTACAAGACCCTAGTCGTAGTGTTGGCTACCACAGGCTATGCTGTCTATTGATGACCCTAGTGTT
GTACTATGCAACATTCCTTGCAACTGTGTGCTCTACACAGCCAAAATATTTTCTCTGGGCGAAATCTTCAGACTGGACATGCA
CCCTGTGGGTTCTGGGCATTACACGGCAGAAATTCCAGTGACGATACTAAGAGAACCTGCTTGGACCAGCATTTCATGTGGAG
TTGTGCAGATATATAATTGTCCTCTCTGGATGTCTGGGTAGCTTTGGTTATTCCATGCTCCAAATGGTAGAGAGGATAGCATG
CATGTTTTGGACAAAAACGAATAAAACCACTGAAGGCTAGTAGGAAAATTCTTCTTGATGGGTTTTCGTAAGATGTGGCAATT
ATAATATTCCTCTGATAAATATATATATGGCTAGTAGGAAACTTTATAAGTATATATATGGCAGTCAGTTTGGTGAAATGGAG
TTTTAGTGTTGCACATATTTTCTTTTCCCTGAAATTGATTTCGATGAATAAATTAATTGGACTACTTTTGTTTTTTCTTGCAA
TTGAGAAATGAGTACACCATTTAATTCACATTTTGCAGTCGGACCTGTTTGTTATGAATGTCAATATGGAACATCCACTAACA

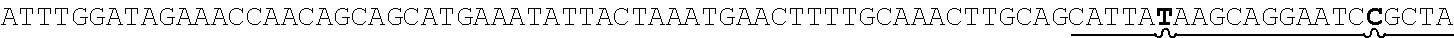

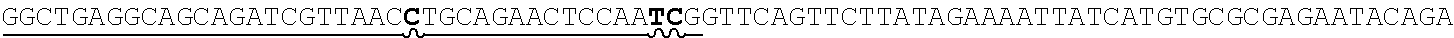

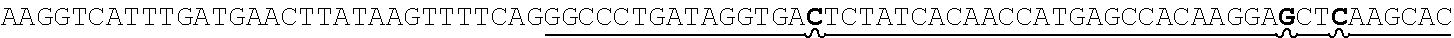

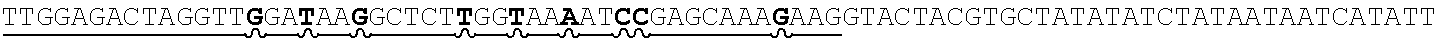

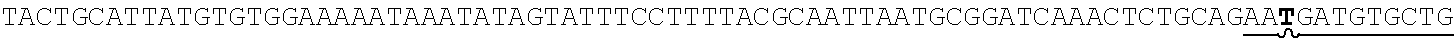

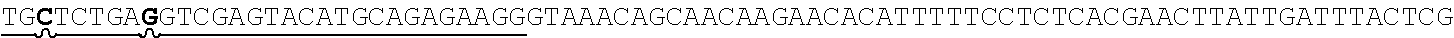

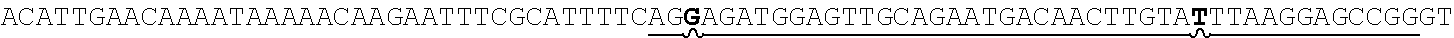

AAGTAACCTGTAGTTGCTAATTAAGAAGGACAGTCTTAGAAGCAAAATATGGAACTGTAATTACTAAAATGCCAAGTTTACCT

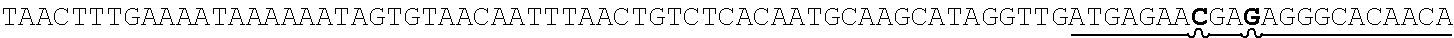

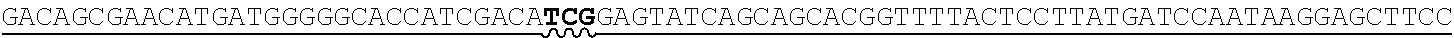

TGCAGTTCAACATCGTGCAGCAGCCTCAGTTCTATTCTCAGCAGGAGGACCGGAAAGACTTCAACGACCAAGGTAGATTTTTT
TTTTATATCTACCACATTTATTTGAAATGTATTTAAATGGACACCCTAAGGTATTTCATAATACCATGTATATTTTTCTAAAT
GAAATAATAATCCCTCCATCTCAAATTATTATTCATTTTAATTCTTGGTTTTTAAGAATGATGATGAATCTAGACACATACAT
CAATTATTAACTGTACGAATCTATTAGAAGTTTAAAATGAATTTTAATTTGGGACAGAGACAATATTTATTAGTCATCACAAA
ACTTGGAAGCCATAGGGCTAGGAAAACAAAATCATTGCAGTTAGTTTTAAATAAAATGCCTAAACCTTCCACTTGGCTATATA
TATATATATATATATATATATATATATATATATATATATATATATATATATATATATATATATATATATATATATATATATAT
ATATATATATATATATATATATATATATATATATATATAATTTCCTCTCCAAGTACATGTTCTGCTATTCTGATTCCTTTTAA
TGTCTTTAGGTGGAAGATAAATATTGGACCTCTCAAGCTTCAGTACTTATCCGTGATGATGCATGACTGCCAGTGAGAAACTG
AGCTACATTACTGTGGAATTATATGTAAAGACTAGTACTACTGCTTCATATATGTGCATGTGCGCACACGCGCACGTAGTATG
CACAATTTCATCCCACTATTATGGCTTGGCACCAACTATGTCTCCTTAATTATCATAGGAGAAAAATAAGTCACGCACAAAAA
AATTCTAAAGATGAGGCGCAGTGTTGGATGACTGAAACTGAAAGATAGGATTCTCGTGGATGGCGGTGGTCATATATATGTCA
TGCACATCTGATCTCTGTTCCAAAATGCCACATCTTCTTTCCCAACCCACTCGAATCTACACCGAGAATATATATGTTTTATT
CGTAATGTATAGACACTATTAGCAATGATACTGTGAACTATGGTTATGGTCCACAATAAGAAATGCATATTTACCTGTG

DNA with ZAG1 CDS decreased to 30% with UTR structure constraints, with coding regions underlined and differences from reference in bold with wavy underline

SEQ ID NO: 61 provides a design for a polynucleotide sequence to help identify what needs
DNA editing with CDS trained with Zm00001eb284010_T001 UTR-sequence-and-structure
constraints to a 30% expression design level with putative encoding of a
Zm00001eb284010_P001 variant of a ZAG1 protein of Zea mays
GGAGCAAGAAACACTCAGAAGCTGCCCAGAGCTACCACCCTTCTTATCCCCACCCCTCCTCCTCCTACCTTTTCTCCTTCAGA

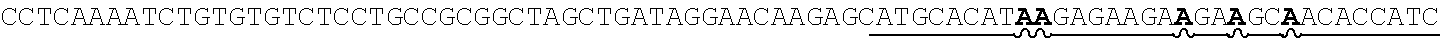

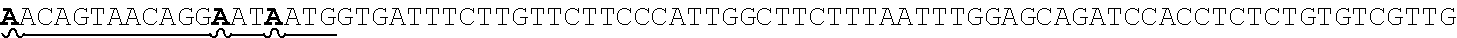

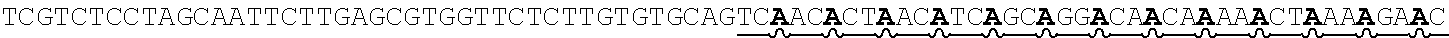

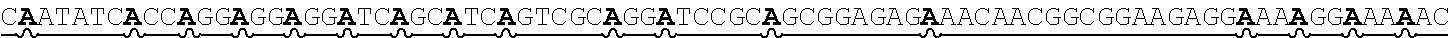

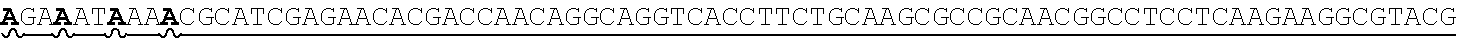

AGCTCTCCGTGCTCTGCGACGCCGAGGTCGCGCTCATCGTCTTCTCCAGCCGCGGCCGCCTCTACGAGTACGCCAACAACAGG
TCTCTTCTTTCTCTCCCCAGCTCTGTGCATCTCTCTCTCTGTGTGGTACTAGCTAGCTAGTACCGCAATATAATGGTAGTAGG
CGCTACTACGACGAATCACCTTCGCTCCAAGAAATGGTAGGCTACATATATAAGTGATGAGTTTGTCGTCCCGTCTAGCTAGC
TAGACACATGATGTGTATTTCGACGACGCTCTCCGAGAATTGAAGCAGCAAGTTAGCTCCCAGCTTTCTTGCTTGCAAAGAGG
AAAGCTGCATCTCGTTTGCTGCTACGACTACATGCGTTCTGTTTATTAACTGTCTTGCTTATTTTTTGTGCTACCGCGGAGAG
GGGCGAAAGAAATCGCAACGGTTTCTTTGCGCGCGTGCGTGCTCCGTCATCCTTTCCGGTTTCTCTTGTGTTCCTGTGAGGTG
AGCTAGATGTCTAGATAGATTCAGCGCAGGGGTATGCTTTGTGAGGTGAGCTTTTCTAGTGCGCGTGCGTGCAATGGGCGGGT
GCCTTGCGGTTGCTTTGGTTTGGTGTGGTGGTCCGCTTCTTTTCTTGCTCCATTTAATTATTTCTTTCCTTTCTATGATCTAT
ATATCCCTGAGGAATATCAGTGAAGACTTATTAGATCTCTCATACCTCATGATACATAGCAAGCATTGTTCTCACACAAGGAA
AAAAAACTTTTTGTTGCTTTGGTCATGTATATATGTGCAGATCTAGTAATTATTATCCTTCGCTCTTAGATTTGGGTCACTAG
CTGGATGTGCGCGCGTGCAGTAACTCGGTAACTGGATGCAAACTTGTGAATGTGAGTGAGGGGAGGTAAGCATTTGAGACGTA
AAAAGATGCCAGGCGCGCATCCTGTCCTGCTAGTGCTATATGATGTTAGGGTTAGGGTTCCATCCCATGTGCGTCCGTCGTCA
TCATCACCATCTGGCCTCTCTCTCTCTCTTCTCTCTCTCTCTCTCTCTCTCTCTCTCTCTCTCTCTCTCTCATGCTGTCATAC
ATTGTGTTTTCTGTAGATGTTACTGTTGTCCCTGCGTGTGTGGTGGTTCTGCAGGTCTCCTCGTCACAGCCATGTATATCTGC
GCGCGCGCTGTGCACTGTGCTGCATATGCATATGCTAAAGTGATGCGCAACTGCCCATCACGGGAGCTCTGTCCTCTCTGCCA
CCACATAGCACTGTAGTTTTTAGGACTGGCAGGAAATAAAAGGGGGGTAATGTAATGCAGAAGGAAACGATAACATCAAATTG
AAAACAAAGAAGCAACCTAGAAACAGCGTGATAGCAAATTAAATTTCCTTAGGCATTTGTGCTTATTTTTTAAGATTTCTTTA
TCACATCCTTATGTTGCACACGTACATGCAATGCAAGTGGCATGAGTGAACGAATATATAGATTCATTACAGCTTTGACAAAT
TGGAACCACTCCATCTGATTGAATGCGTTTGACTTGTAAACTCAAAGGACAATATGTGTTGTGTTTCTTCGCAGAAATTTGAG
CATTATGATTGGGTTCTCGTATCTTCAAGTGAATATTTACTGTTAATAGTAATAGTAGTGTTCATCACACTGAACCATAAATT
TGAGAGAAACACCATTTTGTAAGTAATCAAATTACTTTTCTTGAACAGATTCCAAAAGAATCAAAATTTCATCTATATTGTTG
CAATATTGAAACTAAAAAGTAGCTCGGATGCATTAGTCAACATGGTATTGCAAATATTTGTACAATAAAATCATAGTTTTGAA
ACTGTTGTATCTATATGGTGTTTGATTGGTTACTTGTGTTTGCAATGTATTCAGGCAGTGCAGATTGGTTGTATTTGGTTTGC
CAGACATATATAGTCTCTTATTCATGCTTGCACTTTGCTCAAAGAAAGCACAATAATGCATATTAGCTAGTATGCCAGATTTG
GTCGTACTTAGACAAGGTTTGCACAATCTGATGGGATGGTCATACAAAAATAAAACAACCTCTTCCCTTTACATATACTAAAA
ATATGAGAACTCAAATAGAACATGGATCGGAGGTAGCAATAAAGTGTGAAGAACCAAACATATTTTTTATACACTGCATGTGT
TTCTGATCGACTCAGGTTGCATTGCATAAGGTCTAATGCAGAGAAGACTAATGCAAGCAAACAATTAGAGCCATAACATCTTA
TAATTGATATAAAAGTATACCTATCTATTTCATGATAATTTTAAATATAAAAGGTTTGCTCAAATAAAAATGCAAACTATAAT
TGATTTTTTTTTTGCTCAAAGCTCGACGGAAGCCAGATGTTCGAACGGGTGTAGCTGCAATTCATAGAGATAAAACACTGTAG
AAATATTAGAGCGACCTGTACGAATTAAAGTCAATCGACGCTATAGATATATATTTGGTCTTTGAGGGCGTACGGGGCAACTG
AAATGTGTGCCAATTTGTTTGTGTGATGTGAATGCTCTGTATACGGAACCAGTAGCATGACCAGTTTTCCTTGCCACTGCCAG
AGTTGTATCGGGGGACTACAAGGGGAAGAAAGCTGGCAATGCAAGCTGGTGTTGGTCAGAAGGGTTATGGCACCCAACGGTAG
GGCTGACCAGTTGGGTAACAGCTGAGTCCATGTCTTATCTATGCAGCACTCCTTTGTGACCAATGGGTAGTAGATGTCCACTG
CTTGCAGGCAATCATGTTGTGGGTAATGCATGGCTACTGCCAAGCATGCTCGAGGATGCTAAGGTTTTTGCTATGTTCACCTA
CAGAGTACAGAACAGCAGTACCTTTGAAATTGGTAGCCATCCGTCTCAAAATTCGTTCTCATTTTCGAAAATGCAACCATTTT
AAACTTACAAATATATATAATAAAATATTAATATTACATAATTAGTATCGTTAGAAGATCTTTGAATCTATTTTCATAACAAA
TATTTTTAAAGATACAAATATTACAAATATTTTTTTATAAATTTAAATCAGTCTTGACAAACTTTGACCTGCACGAAATGACT
TAGACAACTATTTTAGAATGGAGAGAGCATATAAATTATGTGAGTCAAAGAGTTTCTTAGAAATAATGGTTGGCACTAAGTGG
GTTCTACATTATTGCCCCCATAAATATTTCTTTGAAGCACTGCTATTAAAATGCTTGGCCTCCGCGATGGATGTTATGAGTTC
CAAAAAAAGTGACGAGTTCCTGTTAATCTGTGTATGGCAACTGCAAAATTGGCATGTTTAGAAAATACATATCTCGTGTAAGC
ATAGGGTTCAAATTTGAGTCAAGATGCATGATTCATGTCTCATATTTCTTTTAGCTAGAATAAAAGTGGTATTTACATTTCCT
TTTTGAACGTAAGACATTTACATGGATATGTCATCTTTTTCTGACAAATATTTAGACAACTGTTTTTTACCATCAAAGCTAAT
TAATGTGAATAACACATCATTTTTTAGATTAGATTGTTTAATTTTTTGCAATGAAAAAATCATTCCTTTGAAGAATCATGTGA
TCCATCTTTCTCCTTTCACTTCCGTTGCACCATTAACATAAGTTATTTAACAATACAATCCATATGTTCCTTTAAATTTGTTT
ATGTCCTATTTAGTAAAAATATCCACTTAAGCAACACAAAGAGAGCTGGCTCAACTTAACTGTGAATGCATCGAATCCACTAA
CTATTGCGCTTTTGGTTATTATCAACAACAAATATTTTTATTAATATATCAAGATAATCTATATTTTATCAGTTTCTCAATGT
TTCAAGATTTAAAAATATAGGTCTTGAGTTTTTTTTCTTGAGAGGGTAATTTTACCAGGATGTTTTTTGGTTCAGTTCAAAAT
TTAATTCAATCCTAATGTTTAGATCTAGACCCCCTTTTTTTAAACAGTTCTTGTAGGATTTGACTTTGTTGAAGAAAACAAGG
ATGAATTTTTTTGCTATGCTAGCTTAGGAGTTCCGATTTTCTTAAAAAGAACTGTAGTAGTTATTAGGTGTGGTATTACATAG
GTTCAAACTCCATGTATGTGCACTACAAACATATTATGGGTCTGTTTGGTTTAGCTGTAGATTCCTAAAACTCTGATTCTAGC
TATGAGCTGTGAGAAAGCTACTATGAACTGACAATTATGAGAAAACTGAAATTAATTTGGCTGAAACAACTGTCAACTATAGA
TTCTATAAGAGCATGACAGCTTGTAACATAGACATCATGAATCCATAAAATCTAGGGTAGGAGGTGATTCTGAATTGAACTAG
AGGAATCTATAGTTTATCTATTCTACTATTGTATATGTTTGATTGAGATTTCAGATTTTGGACAGCAAATCTAGTTCAGAAAG
CTAAACCAAACATACCATATAAGAACTCAAGTTCTATGTAGATGCATCAGTATTTATTAGAATGCTAACTCAATGGCAGTTAG
GATTTGATTATGCTGGCAATGCGATAGTATACAATATTAGACTATTAGTGAAGAGTGAAGCAGTAATGTAAGCAATGCATGTG
CTTACTAAAACGTGATAAATCATTGTTGCACCGAATATTAATACACAACATTAGGATGTGCTTGCACTTCCTGTTATTATCTG
TAGCATTGACGAGGACCACTCTCTATTTTCACATGACACTAAATAATGTGTTAAAATCAACTACTTCTAAGTGATTGCAGCAG
TATTTACCACACCAAATTAATATCACAAAATAGATTATGAAATATATTTTCATAATTTAACCATTTAGAGTCCTAGATGATGT
TACTATTTAAGTGATTGCTGGGATGGCTTTACTTTAGCATGGAAGTATTAATAGTATTTTGGGACGATGCGTTAGAATATTGG
TTACTTTATGTGCTGATTGTATTGACTTTCACTAATGGGGCTGTTCTGGCTAGATTATTAAAGCCGTTCGGACTGCTGCTGCC
GTATAGACAAAATACTGTAGAAAAGGTAGAAGCCGGTGGATTAAAGCCGAAATACTGTAGAAAAAGTAGAAGCCGGTGGATTA
AAGCCGCAACGGACAGGATGAATGTCAAATTGTAATCGACATACTTGCTAGAAGGCAATGTATCCATTATAATCGCTGGAAGA
TGTCAAATTTCAGTTATTGTATATGTTCTATGAATCTCCATTATAATTTATTATGCTTGTGACGATTGTTCTATTATCTGTTG
TATTTTTTCAAGAAAAATATCTAGTTTTGCCCTGGAACAATTTTGTTTCATATTTTCATTCTTTCGGCACATACAATTATCTC
ACCTGATTGAATGTATGTGTACTAACTACTAACTACAGTATTATATTGTATGTACAGCGTGAAGGGCACCATTGAGAGGTACA
AGAAGGCAACCAGTGACAACTCCAGCGCAGCTGGTACGATTGCAGAGGTCACCATTCAGGTAACATCATGTCGATCAGTCAAC
ACATCGTTTCTGATCCTACAAGACCCTAGTCGTAGTGTTGGCTACCACAGGCTATGCTGTCTATTGATGACCCTAGTGTTGTA
CTATGCAACATTCCTTGCAACTGTGTGCTCTACACAGCCAAAATATTTTCTCTGGGCGAAATCTTCAGACTGGACATGCACCC
TGTGGGTTCTGGGCATTACACGGCAGAAATTCCAGTGACGATACTAAGAGAACCTGCTTGGACCAGCATTTCATGTGGAGTTG
TGCAGATATATAATTGTCCTCTCTGGATGTCTGGGTAGCTTTGGTTATTCCATGCTCCAAATGGTAGAGAGGATAGCATGCAT
GTTTTGGACAAAAACGAATAAAACCACTGAAGGCTAGTAGGAAAATTCTTCTTGATGGGTTTTCGTAAGATGTGGCAATTATA
ATATTCCTCTGATAAATATATATATGGCTAGTAGGAAACTTTATAAGTATATATATGGCAGTCAGTTTGGTGAAATGGAGTTT
TAGTGTTGCACATATTTTCTTTTCCCTGAAATTGATTTCGATGAATAAATTAATTGGACTACTTTTGTTTTTTCTTGCAATTG
AGAAATGAGTACACCATTTAATTCACATTTTGCAGTCGGACCTGTTTGTTATGAATGTCAATATGGAACATCCACTAACAATT
TGGATAGAAACCAACAGCAGCATGAAATATTACTAAATGAACTTTTGCAAACTTGCAGCATTACAAGCAGGAATCTGCTAGGC
TGAGGCAGCAGATCGTTAACTTGCAGAACTCCAACAGGTTCAGTTCTTATAGAAAATTATCATGTGCGCGAGAATACAGAAAG
GTCATTTGATGAACTTATAAGTTTTCAGGGCCCTGATAGGTGATTCTATCACAACCATGAGCCACAAGGAACTTAAGCACTTG
GAGACTAGGTTAGACAAAGCTCTCGGAAAGATTAGAGCAAAAAAGGTACTACGTGCTATATATCTATAATAATCATATTTACT
GCATTATGTGTGGAAAAATAAATATAGTATTTCCTTTTACGCAATTAATGCGGATCAAACTCTGCAGAACGATGTGCTGTGTT
CTGAAGTCGAGTACATGCAGAGAAGGGTAAACAGCAACAAGAACACATTTTTCCTCTCACGAACTTATTGATTTACTCGACAT
TGAACAAAATAAAAACAAGAATTTCGCATTTTCAGGAAATGGAGTTGCAGAATGACAACTTGTACTTAAGGAGCCGGGTAAGT
AACCTGTAGTTGCTAATTAAGAAGGACAGTCTTAGAAGCAAAATATGGAACTGTAATTACTAAAATGCCAAGTTTACCTTAAC
TTTGAAAATAAAAAATAGTGTAACAATTTAACTGTCTCACAATGCAAGCATAGGTTGATGAGAATGAAAGGGCACAACAGACA
GCGAACATGATGGGGGCACCATCGACAAGTGAGTATCAGCAGCACGGTTTTACTCCTTATGATCCAATAAGGAGCTTCCTGCA
GTTCAACATCGTGCAGCAGCCTCAGTTCTATTCTCAGCAGGAGGACCGGAAAGACTTCAACGACCAAGGTAGATTTTTTTTTT
ATATCTACCACATTTATTTGAAATGTATTTAAATGGACACCCTAAGGTATTTCATAATACCATGTATATTTTTCTAAATGAAA
TAATAATCCCTCCATCTCAAATTATTATTCATTTTAATTCTTGGTTTTTAAGAATGATGATGAATCTAGACACATACATCAAT
TATTAACTGTACGAATCTATTAGAAGTTTAAAATGAATTTTAATTTGGGACAGAGACAATATTTATTAGTCATCACAAAACTT
GGAAGCCATAGGGCTAGGAAAACAAAATCATTGCAGTTAGTTTTAAATAAAATGCCTAAACCTTCCACTTGGCTATATATATA
TATATATATATATATATATATATATATATATATATATATATATATATATATATATATATATATATATATATATATATATATAT
ATATATATATATATATATATATATATATATATATAATTTCCTCTCCAAGTACATGTTCTGCTATTCTGATTCCTTTTAATGTC
TTTAGGTGGAAGATAAATATTGGACCTCTCAAGCTTCAGTACTTATCCGTGATGATGCATGACTGCCAGTGAGAAACTGAGCT
ACATTACTGTGGAATTATATGTAAAGACTAGTACTACTGCTTCATATATGTGCATGTGCGCACACGCGCACGTAGTATGCACA
ATTTCATCCCACTATTATGGCTTGGCACCAACTATGTCTCCTTAATTATCATAGGAGAAAAATAAGTCACGCACAAAAAAATT
CTAAAGATGAGGCGCAGTGTTGGATGACTGAAACTGAAAGATAGGATTCTCGTGGATGGCGGTGGTCATATATATGTCATGCA
CATCTGATCTCTGTTCCAAAATGCCACATCTTCTTTCCCAACCCACTCGAATCTACACCGAGAATATATATGTTTTATTCGTA
ATGTATAGACACTATTAGCAATGATACTGTGAACTATGGTTATGGTCCACAATAAGAAATGCATATTTACCTGTG

### J. Example 10: Gene Training as a Supplemental Approach to Mitigate, Treat, or Prevent Genetic Disease

In this example we focus on supplementation of HEXA mRNA to help delay, slow, reduce, mute, or prevent development of autosomal recessive Tay Sachs disease.

### Tay Sachs-Supplemental Preventative Approach

HEX protein sequence:

SEQ ID NO: 62 provides the amino acid
sequence of an HEX protein of Homo sapiens

MTSSRLWFSLLLAAAFAGRATALWPWPQNFQTSDQRYVLYPNNFQFQYDV
SSAAQPGCSVLDEAFQRYRDLLFGSGSWPRPYLTGKRHTLEKNVLVVSVV
TPGCNQLPTLESVENYTLTINDDQCLLLSETVWGALRGLETFSQLVWKSA
EGTFFINKTEIEDFPRFPHRGLLLDTSRHYLPLSSILDTLDVMAYNKLNV
FHWHLVDDPSFPYESFTFPELMRKGSYNPVTHIYTAQDVKEVIEYARLRG
IRVLAEFDTPGHTLSWGPGIPGLLTPCYSGSEPSGTFGPVNPSLNNTYEF
MSTFFLEVSSVFPDFYLHLGGDEVDFTCWKSNPEIQDFMRKKGFGEDFKQ
LESFYIQTLLDIVSSYGKGYVVWQEVFDNKVKIQPDTIIQVWREDIPVNY
MKELELVTKAGFRALLSAPWYLNRISYGPDWKDFYIVEPLAFEGTPEQKA
LVIGGEACMWGEYVDNTNLVPRLWPRAGAVAERLWSNKLTSDLTFAYERL
SHFRCELLRRGVQAQPLNVGFCEQEFEQT

HEXA Reference CDS RNA (−MFE=549.00; [0-1] design level=34.20%):

SEQ ID NO: 63 provides the nucleic acid sequence of a HEXA CDS
encoding a variant of a HEX protein from Homo sapiens

AUGACAAGCUCCAGGCUUUGGUUUUCGCUGCUGCUGGCGGCAGCGUUCGCAGGACGGGCGACGGCCCUCUGGCCCUGGCCUCA
GAACUUCCAAACCUCCGACCAGCGCUACGUCCUUUACCCGAACAACUUUCAAUUCCAGUACGAUGUCAGCUCGGCCGCGCAGC
CCGGCUGCUCAGUCCUCGACGAGGCCUUCCAGCGCUAUCGUGACCUGCUUUUCGGUUCCGGGUCUUGGCCCCGUCCUUACCUC
ACAGGGAAACGGCAUACACUGGAGAAGAAUGUGUUGGUUGUCUCUGUAGUCACACCUGGAUGUAACCAGCUUCCUACUUUGGA
GUCAGUGGAGAAUUAUACCCUGACCAUAAAUGAUGACCAGUGUUUACUCCUCUCUGAGACUGUCUGGGGAGCUCUCCGAGGUC
UGGAGACUUUUAGCCAGCUUGUUUGGAAAUCUGCUGAGGGCACAUUCUUUAUCAACAAGACUGAGAUUGAGGACUUUCCCCGC
UUUCCUCACCGGGGCUUGCUGUUGGAUACAUCUCGCCAUUACCUGCCACUCUCUAGCAUCCUGGACACUCUGGAUGUCAUGGC
GUACAAUAAAUUGAACGUGUUCCACUGGCAUCUGGUAGAUGAUCCUUCCUUCCCAUAUGAGAGCUUCACUUUUCCAGAGCUCA
UGAGAAAGGGGUCCUACAACCCUGUCACCCACAUCUACACAGCACAGGAUGUGAAGGAGGUCAUUGAAUACGCACGGCUCCGG
GGUAUCCGUGUGCUUGCAGAGUUUGACACUCCUGGCCACACUUUGUCCUGGGGACCAGGUAUCCCUGGAUUACUGACUCCUUG
CUACUCUGGGUCUGAGCCCUCUGGCACCUUUGGACCAGUGAAUCCCAGUCUCAAUAAUACCUAUGAGUUCAUGAGCACAUUCU
UCUUAGAAGUCAGCUCUGUCUUCCCAGAUUUUUAUCUUCAUCUUGGAGGAGAUGAGGUUGAUUUCACCUGCUGGAAGUCCAAC
CCAGAGAUCCAGGACUUUAUGAGGAAGAAAGGCUUCGGUGAGGACUUCAAGCAGCUGGAGUCCUUCUACAUCCAGACGCUGCU
GGACAUCGUCUCUUCUUAUGGCAAGGGCUAUGUGGUGUGGCAGGAGGUGUUUGAUAAUAAAGUAAAGAUUCAGCCAGACACAA
UCAUACAGGUGUGGCGAGAGGAUAUUCCAGUGAACUAUAUGAAGGAGCUGGAACUGGUCACCAAGGCCGGCUUCCGGGCCCUU
CUCUCUGCCCCCUGGUACCUGAACCGUAUAUCCUAUGGCCCUGACUGGAAGGAUUUCUACAUAGUGGAACCCCUGGCAUUUGA
AGGUACCCCUGAGCAGAAGGCUCUGGUGAUUGGUGGAGAGGCUUGUAUGUGGGGAGAAUAUGUGGACAACACAAACCUGGUCC
CCAGGCUCUGGCCCAGAGCAGGGGCUGUUGCCGAAAGGCUGUGGAGCAACAAGUUGACAUCUGACCUGACAUUUGCCUAUGAA
CGUUUGUCACACUUCCGCUGUGAAUUGCUGAGGCGAGGUGUCCAGGCCCAACCCCUCAAUGUAGGCUUCUGUGAGCAGGAGUU
UGAACAGACCUGA

HEXA CDS RNA via CDSFold with Maximized −MFE:

SEQ ID NO: 64 provides a design for a nucleic acid HEXA CDS sequence trained to a
100% expression design level encoding a variant of a HEX protein from Homo sapiens

AUGACUUCUUCACGUCUGUGGUUCAGCCUCCUGCUCGCAGCGGCGUUUGCCGGUCGGGCUACGGCCUUGUGGCCAUGGCCCCA
AAACUUCCAGACAUCAGAUCAGCGGUAUGUGCUGUAUCCGAACAAUUUUCAAUUCCAGUACGAUGUGAGCAGCGCGGCACAGC
CGGGCUGCUCCGUACUGGAUGAAGCGUUCCAGCGCUACCGCGAUCUGUUGUUUGGAAGUGGGUCAUGGCCACGGCCGUACCUG
ACCGGCAAACGCCACACCCUGGAGAAGAAUGUUCUUGUUGUCAGCGUAGUCACCCCCGGGUGCAAUCAGCUUCCCACCCUUGA
GAGUGUAGAAAACUACACUCUCACUAUUAACGAUGACCAGUGCCUUCUGCUCUCGGAGACCGUGUGGGGGGCUCUACGCGGGC
UGGAGACCUUUAGCCAACUGGUGUGGAAGAGCGCGGAAGGGACUUUCUUCAUCAAUAAGACGGAGAUUGAGGACUUUCCGCGC
UUUCCACACCGGGGGCUGUUGUUGGACACCUCCCGCCACUAUCUCCCGCUGUCGAGCAUACUCGACACCCUUGACGUCAUGGC
GUAUAAUAAGUUGAAUGUGUUUCACUGGCAUUUAGUUGACGACCCCUCUUUUCCGUAUGAGUCCUUUACUUUUCCGGAACUCA
UGCGGAAGGGGUCGUACAACCCAGUGACGCACAUUUAUACUGCGCAGGACGUCAAGGAGGUGAUCGAGUAUGCUCGACUGCGG
GGGAUACGUGUGCUCGCGGAGUUUGAUACUCCCGGGCACACGCUCUCAUGGGGUCCUGGAAUCCCCGGGUUGCUCACCCCGUG
UUAUUCAGGCUCGGAGCCAUCGGGCACUUUUGGCCCGGUGAAUCCGAGCCUGAAUAACACGUACGAGUUUAUGUCCACCUUCU
UCCUCGAGGUGAGUAGUGUCUUCCCUGACUUCUACCUUCACCUCGGGGGAGAUGAGGUGGACUUUACUUGUUGGAAGAGCAAC
CCGGAGAUCCAGGACUUCAUGAGAAAGAAGGGUUUUGGGGAGGACUUCAAGCAAUUGGAGUCCUUCUACAUUCAGACCCUUCU
CGACAUAGUCUCUUCCUACGGGAAGGGCUAUGUCGUGUGGCAGGAGGUGUUCGACAACAAGGUCAAAAUCCAGCCCGAUACGA
UCAUCCAGGUCUGGCGCGAGGAUAUCCCGGUUAAUUAUAUGAAGGAGCUGGAGCUGGUCACGAAAGCAGGCUUUCGUGCCCUG
CUCUCAGCUCCUUGGUAUCUUAACCGGAUAUCCUACGGGCCAGACUGGAAGGAUUUUUAUAUCGUAGAGCCCCUCGCAUUUGA
AGGUACUCCGGAGCAGAAGGCACUGGUCAUCGGGGGGGAAGCUUGCAUGUGGGGGGAGUACGUUGACAACACGAACCUUGUUC
CCAGGUUGUGGCCCCGCGCAGGGGCUGUGGCUGAACGCCUCUGGAGCAACAAGCUCACAUCGGAUUUGACUUUUGCGUACGAG
CGCUUAAGUCAUUUCCGAUGUGAGCUGUUGCGCAGAGGCGUUCAGGCACAGCCCCUGAACGUGGGGUUCUGCGAGCAGGAGUU
UGAGCAGACGUGA

HEXA 5′UTR—

SEQ ID NO: 65 provides the nucleic acid sequence of a HEXA 5′ untranslated region
of Homo sapiens

CUCACGUGGCCAGCCCCCUCCGAGAGGGGAGACCAGCGGGCC

HEXA 3′UTR—

SEQ ID NO: 66 provides the nucleic acid sequence of a HEXA 3′ untranslated region
of Homo sapiens

GCCCCAGGCACCGAGGAGGGUGCUGGCUGUAGGUGAAUGGUAGUGGAGCCAGGCUUCCACUGCAUCCUGGCCAGGGGACGGAG
CCCCUUGCCUUCGUGCCCCUUGCCUGCGUGCCCCUGUGCUUGGAGAGAAAGGGGCCGGUGCUGGCGCUCGCAUUCAAUAAAGA
GUAAUGUGGCAUUUUUCUAUAAUAAACAUGGAUUACCUGUGUUUAAAAAAAAAAGUGUGAAUGGCGUUAGGGUAAGGGCACAG
CCAGGCUGGAGUCAGUGUCUGCCCCUGAGGUCUUUUAAGUUGAGGGCUGGGAAUGAAACCUAUAGCCUUUGUGCUGUUCUGCC
UUGCCUGUGAGCUAUGUCACUCCCCUCCCACUCCUGACCAUAUUCCAGACACCUGCCCUAAUCCUCAGCCUGCUCACUUCACU
UCUGCAUUAUAUCUCCAAGGCGUUGGUAUAUGGAAAAAGAUGUAGGGGCUUGGAGGUGUUCUGGACAGUGGGGAGGGCUCCAG
ACCCAACCUGGUCACAGAAGAGCCUCUCCCCCAUGCAUACUCAUCCACCUCCCUCCCCUAGAGCUAUUCUCCUUUGGGUUUCU
UGCUGCUUCAAUUUUAUACAACCAUUAUUUAAAUAUUAUUAAACACAUAUUGUUCUCUAGGCACUGUGGUAGUGGGUUUUUUU
GUUGUUUUUGUUUUUGAGACUGUCUCAAAAACUCUGUCGCCCAGGCUGACAGUGCAGUGGCACAAUCUUGGCUCACUGCAGCC
UCUGCCUCCUGGGUUCAAGCGAUUCUCGUGCAUCAGCCUCCUGAGUAACUGGAAUUAAUAGGCACGUGCCACCAUGUCCAUCU
AAUUCAUAUAUAUAUAUUUUUUUUUUCUGAGACGGAGUCUCACUGUCACACAGGCUGGAGUGCAGUGGCACGAUCUCGACUCA
CUGCAAGCUCCACCUCCUGGGUUCACGCCAUUCUCCUGCCUCAGCCUCCCCAGUAGCUGGGACUACAGGCGCCCGCCACCACG
CCCGGCUAAUUUUUUGUAUUUUUAGUAGAGAUGGGGUUUCUCCGUGUUAGCCAGGAUGGUCUCGAUCUCCUGACCUCGUGAUC
CGCCCGCCUUGGCCUCCCAAAGUGCUGGGAUUACAGGCGUGAGCCACCGCGCCCGGCCGAAUUCAUCUAUUUUUAGUAGAGAU
GGGGUUUCACUAUAUUGGCCAGGCUGGUCUUGAACUCAUGACCUCAGAUGUUCACUUGUCUUGGCCUCCCAAAGUGCUGGGAU
UAGAGGCGUGAGCCACCGCACGCGGGCCUGUGGUAAAUUGUUGAAUUUGAAGGACUCAGAGGCCCUGGUCAAUUCCAAAAUAA
CGUAGGCGACUUCCAUCCCCCUCCUCCCAACCAUUUUCAGCCCAAAGCAUCUUCGCAGGGAAUGGAUGGCUGCGCGGAGGUGG
GCGGUGGCUCUGGAGAGGGUCUUUGCAGGUGUGAUUUUCUCUAGAAGGAAAUGUCUCGUCGUGGACCCAGACUGCCCCCUCCU
GGUUUCAGAUGCAGAAGUGAUACUGUAAGCCAGAGGCGGGGGCAGUAAUGCAUCGCAGCCAUUUUAGGUGAGGAUUUCCUUGG
CGGUUAUUUGUUAAGUUCUUUGGCUGGGCCCUGGGCUGGGGUAACAAUGGACAGGUUCCAGGCAUUUUUUUCAGAAAGCUUCC
AGUGUAGUGGAUACAGAAACUUCAGGAAGGCAGGGCUGAGAAGGAUCUGAGUAAAACUCGGUCCUUCAACACCAUCCUUCAGC
CCCUGGGUCAUGUUCCUUCGAGGUCCUGGUGGGAGGUAGACAAGCCUAGCCUUGUGCUGUUCCUGUAAGGACAGGGUGGGCAU
UUUCUACCAACAGAAUUCUUGGAAUUUUCACACAGCCCAGCCUAGCCAAGUCCAGGGCUAUAGCCCAGAUACACAAGUUAAGG
UCCCAGCACUGGCACCCACCACAGGAGCCCCCUUACCUCUAUUACCCAGAAGCUUGUAGGAGGGGUGGUCCGCAGACAAGGAC
CCUGCACAGGUGCGACCCUGCUUCCCUCCUGGUCAUAACUUUCAUGUUACUAUUGCUUGGGAUAAUGUUAAGUAAAAAUAGCA
GACACUGAGUUUUAAGUCUCAAGUGGAUGAAGGCAGAGAUCGUGAUACACUUGAGUUAAAGCAGUAGGGUUCUGUCAUUUUCU
AUUCCUGUUGUAAACAUUUUCUUUAAUGUUAUUAUUUUUACCACUAAACUAACGUGGCCUGGUCACGACUUUCAUUGGUAAAG
UGUGCUGUUCCUCACCCUCCACCGUUGCUCCUUUGGUCCACUGAUCAUAAGAGCAUUUACCUGAAGGUCGUCAGACCUCGAAU
GCCAACAGGUCAACUGCAGUGGCCUGCAGUUACCACCCAGUCUGUUCCAAUGAACAGAAUCGCUGUUGCCCCAACUCAUCUCC
CUUCACCUAGGCUGUAAAUUGAAAGUCCCACCCCUGAGCGGAACACAGGCCAUCUUGUGUGCUGUGCACCACCAGGGGGUGGG
GAAGUUUCCAGACUGACUUCCUGGCUCCAGUCAUCCUAGGAAAAGAGUUCUCCAGUCGCUCCCCACCCCCACCCCUUCCCAUU
CCAGGAGUCUAUUAAGGAGGCAAAGCAGGCCUAACGGGUAUCAAAGCAAAGGAGUGAAUGGAGACUGGGAGAGUCUUCAACCU
CUCCUCUCCUUGGUAGGAGCUGAGGCUGCAUGCCAGGUACCUUCCCUUCGAGGAAUCUAAUAAAGCUAGGUCACUGGUGUUUU
CAGGUGCUUCUCAAAGGAUUGCCGUAGGGGUAGGAUAUCAGGAUGUGGGAGCACAGGUGCCACCACAGCACUAGUGAUGGAGA
GUCAUUGCCCCUAGACUUCUGGGACAGUGAGACUGUGAGGAAAGCUGAAAUGAUACUGGGAAAGGGUGAAAGAAAGGAUGUAG
GUGGAAUUUAUUUAGUAUUAAUGUAGGUACACAUACCUUAUGGCAACAUUCCUAGCACUCUAAAUUCUAGAUUUGUAUAGUUU
CUGUCAAUAUCUUUUGUAAGCUUAAUCAAUACAGGGCAUGACAAGUAUGUGUCACAUACUUUUUUUUCCACGAAGAAAAAAAA
UAAGUAGGAAUUGGGUGCUUUGUUUAUCAAAAUUUGUAUUUCCUUUAUAAAUAAACUUUGAAAUAAAGGUUGAAAAUUAGUA

HEX protein with CDSFold-designed CDS with accordingly maximum −MFE [0-1] design level @ 100% expression (−MFE=2232)

SEQ ID NO: 67 provides a design for a nucleic acid HEXA mRNA sequence with CDS
trained to a 100% expression design level encoding a variant of a HEX
protein of Homo sapiens

CUCACGUGGCCAGCCCCCUCCGAGAGGGGAGACCAGCGGGCCAUGACUUCUUCACGUCUGUGGUUCAGCCUCCUGCUCGCAGC
GGCGUUUGCCGGUCGGGCUACGGCCUUGUGGCCAUGGCCCCAAAACUUCCAGACAUCAGAUCAGCGGUAUGUGCUGUAUCCGA
ACAAUUUUCAAUUCCAGUACGAUGUGAGCAGCGCGGCACAGCCGGGCUGCUCCGUACUGGAUGAAGCGUUCCAGCGCUACCGC
GAUCUGUUGUUUGGAAGUGGGUCAUGGCCACGGCCGUACCUGACCGGCAAACGCCACACCCUGGAGAAGAAUGUUCUUGUUGU
CAGCGUAGUCACCCCCGGGUGCAAUCAGCUUCCCACCCUUGAGAGUGUAGAAAACUACACUCUCACUAUUAACGAUGACCAGU
GCCUUCUGCUCUCGGAGACCGUGUGGGGGGCUCUACGCGGGCUGGAGACCUUUAGCCAACUGGUGUGGAAGAGCGCGGAAGGG
ACUUUCUUCAUCAAUAAGACGGAGAUUGAGGACUUUCCGCGCUUUCCACACCGGGGGCUGUUGUUGGACACCUCCCGCCACUA
UCUCCCGCUGUCGAGCAUACUCGACACCCUUGACGUCAUGGCGUAUAAUAAGUUGAAUGUGUUUCACUGGCAUUUAGUUGACG
ACCCCUCUUUUCCGUAUGAGUCCUUUACUUUUCCGGAACUCAUGCGGAAGGGGUCGUACAACCCAGUGACGCACAUUUAUACU
GCGCAGGACGUCAAGGAGGUGAUCGAGUAUGCUCGACUGCGGGGGAUACGUGUGCUCGCGGAGUUUGAUACUCCCGGGCACAC
GCUCUCAUGGGGUCCUGGAAUCCCCGGGUUGCUCACCCCGUGUUAUUCAGGCUCGGAGCCAUCGGGCACUUUUGGCCCGGUGA
AUCCGAGCCUGAAUAACACGUACGAGUUUAUGUCCACCUUCUUCCUCGAGGUGAGUAGUGUCUUCCCUGACUUCUACCUUCAC
CUCGGGGGAGAUGAGGUGGACUUUACUUGUUGGAAGAGCAACCCGGAGAUCCAGGACUUCAUGAGAAAGAAGGGUUUUGGGGA
GGACUUCAAGCAAUUGGAGUCCUUCUACAUUCAGACCCUUCUCGACAUAGUCUCUUCCUACGGGAAGGGCUAUGUCGUGUGGC
AGGAGGUGUUCGACAACAAGGUCAAAAUCCAGCCCGAUACGAUCAUCCAGGUCUGGCGCGAGGAUAUCCCGGUUAAUUAUAUG
AAGGAGCUGGAGCUGGUCACGAAAGCAGGCUUUCGUGCCCUGCUCUCAGCUCCUUGGUAUCUUAACCGGAUAUCCUACGGGCC
AGACUGGAAGGAUUUUUAUAUCGUAGAGCCCCUCGCAUUUGAAGGUACUCCGGAGCAGAAGGCACUGGUCAUCGGGGGGGAAG
CUUGCAUGUGGGGGGAGUACGUUGACAACACGAACCUUGUUCCCAGGUUGUGGCCCCGCGCAGGGGCUGUGGCUGAACGCCUC
UGGAGCAACAAGCUCACAUCGGAUUUGACUUUUGCGUACGAGCGCUUAAGUCAUUUCCGAUGUGAGCUGUUGCGCAGAGGCGU
UCAGGCACAGCCCCUGAACGUGGGGUUCUGCGAGCAGGAGUUUGAGCAGACGUGAGCCCCAGGCACCGAGGAGGGUGCUGGCU
GUAGGUGAAUGGUAGUGGAGCCAGGCUUCCACUGCAUCCUGGCCAGGGGACGGAGCCCCUUGCCUUCGUGCCCCUUGCCUGCG
UGCCCCUGUGCUUGGAGAGAAAGGGGCCGGUGCUGGCGCUCGCAUUCAAUAAAGAGUAAUGUGGCAUUUUUCUAUAAUAAACA
UGGAUUACCUGUGUUUAAAAAAAAAAGUGUGAAUGGCGUUAGGGUAAGGGCACAGCCAGGCUGGAGUCAGUGUCUGCCCCUGA
GGUCUUUUAAGUUGAGGGCUGGGAAUGAAACCUAUAGCCUUUGUGCUGUUCUGCCUUGCCUGUGAGCUAUGUCACUCCCCUCC
CACUCCUGACCAUAUUCCAGACACCUGCCCUAAUCCUCAGCCUGCUCACUUCACUUCUGCAUUAUAUCUCCAAGGCGUUGGUA
UAUGGAAAAAGAUGUAGGGGCUUGGAGGUGUUCUGGACAGUGGGGAGGGCUCCAGACCCAACCUGGUCACAGAAGAGCCUCUC
CCCCAUGCAUACUCAUCCACCUCCCUCCCCUAGAGCUAUUCUCCUUUGGGUUUCUUGCUGCUUCAAUUUUAUACAACCAUUAU
UUAAAUAUUAUUAAACACAUAUUGUUCUCUAGGCACUGUGGUAGUGGGUUUUUUUGUUGUUUUUGUUUUUGAGACUGUCUCAA
AAACUCUGUCGCCCAGGCUGACAGUGCAGUGGCACAAUCUUGGCUCACUGCAGCCUCUGCCUCCUGGGUUCAAGCGAUUCUCG
UGCAUCAGCCUCCUGAGUAACUGGAAUUAAUAGGCACGUGCCACCAUGUCCAUCUAAUUCAUAUAUAUAUAUUUUUUUUUUCU
GAGACGGAGUCUCACUGUCACACAGGCUGGAGUGCAGUGGCACGAUCUCGACUCACUGCAAGCUCCACCUCCUGGGUUCACGC
CAUUCUCCUGCCUCAGCCUCCCCAGUAGCUGGGACUACAGGCGCCCGCCACCACGCCCGGCUAAUUUUUUGUAUUUUUAGUAG
AGAUGGGGUUUCUCCGUGUUAGCCAGGAUGGUCUCGAUCUCCUGACCUCGUGAUCCGCCCGCCUUGGCCUCCCAAAGUGCUGG
GAUUACAGGCGUGAGCCACCGCGCCCGGCCGAAUUCAUCUAUUUUUAGUAGAGAUGGGGUUUCACUAUAUUGGCCAGGCUGGU
CUUGAACUCAUGACCUCAGAUGUUCACUUGUCUUGGCCUCCCAAAGUGCUGGGAUUAGAGGCGUGAGCCACCGCACGCGGGCC
UGUGGUAAAUUGUUGAAUUUGAAGGACUCAGAGGCCCUGGUCAAUUCCAAAAUAACGUAGGCGACUUCCAUCCCCCUCCUCCC
AACCAUUUUCAGCCCAAAGCAUCUUCGCAGGGAAUGGAUGGCUGCGCGGAGGUGGGCGGUGGCUCUGGAGAGGGUCUUUGCAG
GUGUGAUUUUCUCUAGAAGGAAAUGUCUCGUCGUGGACCCAGACUGCCCCCUCCUGGUUUCAGAUGCAGAAGUGAUACUGUAA
GCCAGAGGCGGGGGCAGUAAUGCAUCGCAGCCAUUUUAGGUGAGGAUUUCCUUGGCGGUUAUUUGUUAAGUUCUUUGGCUGGG
CCCUGGGCUGGGGUAACAAUGGACAGGUUCCAGGCAUUUUUUUCAGAAAGCUUCCAGUGUAGUGGAUACAGAAACUUCAGGAA
GGCAGGGCUGAGAAGGAUCUGAGUAAAACUCGGUCCUUCAACACCAUCCUUCAGCCCCUGGGUCAUGUUCCUUCGAGGUCCUG
GUGGGAGGUAGACAAGCCUAGCCUUGUGCUGUUCCUGUAAGGACAGGGUGGGCAUUUUCUACCAACAGAAUUCUUGGAAUUUU
CACACAGCCCAGCCUAGCCAAGUCCAGGGCUAUAGCCCAGAUACACAAGUUAAGGUCCCAGCACUGGCACCCACCACAGGAGC
CCCCUUACCUCUAUUACCCAGAAGCUUGUAGGAGGGGUGGUCCGCAGACAAGGACCCUGCACAGGUGCGACCCUGCUUCCCUC
CUGGUCAUAACUUUCAUGUUACUAUUGCUUGGGAUAAUGUUAAGUAAAAAUAGCAGACACUGAGUUUUAAGUCUCAAGUGGAU
GAAGGCAGAGAUCGUGAUACACUUGAGUUAAAGCAGUAGGGUUCUGUCAUUUUCUAUUCCUGUUGUAAACAUUUUCUUUAAUG
UUAUUAUUUUUACCACUAAACUAACGUGGCCUGGUCACGACUUUCAUUGGUAAAGUGUGCUGUUCCUCACCCUCCACCGUUGC
UCCUUUGGUCCACUGAUCAUAAGAGCAUUUACCUGAAGGUCGUCAGACCUCGAAUGCCAACAGGUCAACUGCAGUGGCCUGCA
GUUACCACCCAGUCUGUUCCAAUGAACAGAAUCGCUGUUGCCCCAACUCAUCUCCCUUCACCUAGGCUGUAAAUUGAAAGUCC
CACCCCUGAGCGGAACACAGGCCAUCUUGUGUGCUGUGCACCACCAGGGGGUGGGGAAGUUUCCAGACUGACUUCCUGGCUCC
AGUCAUCCUAGGAAAAGAGUUCUCCAGUCGCUCCCCACCCCCACCCCUUCCCAUUCCAGGAGUCUAUUAAGGAGGCAAAGCAG
GCCUAACGGGUAUCAAAGCAAAGGAGUGAAUGGAGACUGGGAGAGUCUUCAACCUCUCCUCUCCUUGGUAGGAGCUGAGGCUG
CAUGCCAGGUACCUUCCCUUCGAGGAAUCUAAUAAAGCUAGGUCACUGGUGUUUUCAGGUGCUUCUCAAAGGAUUGCCGUAGG
GGUAGGAUAUCAGGAUGUGGGAGCACAGGUGCCACCACAGCACUAGUGAUGGAGAGUCAUUGCCCCUAGACUUCUGGGACAGU
GAGACUGUGAGGAAAGCUGAAAUGAUACUGGGAAAGGGUGAAAGAAAGGAUGUAGGUGGAAUUUAUUUAGUAUUAAUGUAGGU
ACACAUACCUUAUGGCAACAUUCCUAGCACUCUAAAUUCUAGAUUUGUAUAGUUUCUGUCAAUAUCUUUUGUAAGCUUAAUCA
AUACAGGGCAUGACAAGUAUGUGUCACAUACUUUUUUUUCCACGAAGAAAAAAAAUAAGUAGGAAUUGGGUGCUUUGUUUAUC
AAAAUUUGUAUUUCCUUUAUAAAUAAACUUUGAAAUAAAGGUUGAAAAUUAGUA

An mRNA sequence with maximum −MFE while preserving UTR sequences via CDSFold variant (−MFE=2243). Please note that there are differences between sequence generation with UTR sequence constraints and without UTR sequences when using the CDSFold variant. The differences between the mRNA sequence produced with the CDSFold variant and the HEXA mRNA reference sequence are highlighted below in bold with wavy underline

SEQ ID NO: 68 provides a design for a nucleic acid HEXA mRNA sequence trained with HEXA
UTR-sequence constraints to a 100% expression design level encoding a variant of a HEX
protein of Homo sapiens

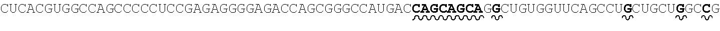

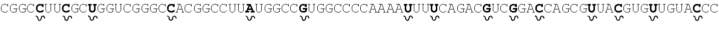

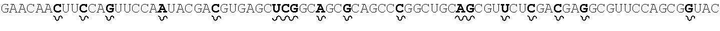

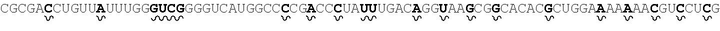

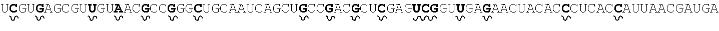

CCAGUGCCUUCUGCUCUCGGAGACCGUGUGGGGGGCUCUACGCGGGCUGGAGACCUUUAGCCAACUGGUGUGGAAGAGCGCGG
AAGGGACUUUCUUCAUCAAUAAGACGGAGAUUGAGGACUUUCCGCGCUUUCCACACCGGGGGCUGUUGUUGGACACCUCCCGC
CACUAUCUCCCGCUGUCGAGCAUACUCGACACCCUUGACGUCAUGGCGUAUAAUAAGUUGAAUGUGUUUCACUGGCAUUUAGU
UGACGACCCCUCUUUUCCGUAUGAGUCCUUUACUUUUCCGGAACUCAUGCGGAAGGGGUCGUACAACCCAGUGACGCACAUUU
AUACUGCGCAGGACGUCAAGGAGGUGAUCGAGUAUGCUCGACUGCGGGGGAUACGUGUGCUCGCGGAGUUUGAUACUCCCGGG
CACACGCUCUCAUGGGGUCCUGGAAUCCCCGGGUUGCUCACCCCGUGUUAUUCAGGCUCGGAGCCAUCGGGCACUUUUGGCCC
GGUGAAUCCGAGCCUGAAUAACACGUACGAGUUUAUGUCCACCUUCUUCCUCGAGGUGAGUAGUGUCUUCCCUGACUUCUACC
UUCACCUCGGGGGAGAUGAGGUGGACUUUACUUGUUGGAAGAGCAACCCGGAGAUCCAGGACUUCAUGAGAAAGAAGGGUUUU
GGGGAGGACUUCAAGCAAUUGGAGUCCUUCUACAUUCAGACCCUUCUCGACAUAGUCUCUUCCUACGGGAAGGGCUAUGUCGU
GUGGCAGGAGGUGUUCGACAACAAGGUCAAAAUCCAGCCCGAUACGAUCAUCCAGGUCUGGCGCGAGGAUAUCCCGGUUAAUU
AUAUGAAGGAGCUGGAGCUGGUCACGAAAGCAGGCUUUCGUGCCCUGCUCUCAGCUCCUUGGUAUCUUAACCGGAUAUCCUAC

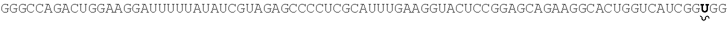

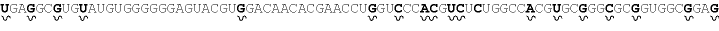

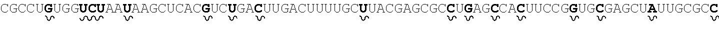

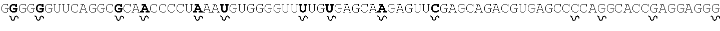

UGCUGGCUGUAGGUGAAUGGUAGUGGAGCCAGGCUUCCACUGCAUCCUGGCCAGGGGACGGAGCCCCUUGCCUUCGUGCCCCU
UGCCUGCGUGCCCCUGUGCUUGGAGAGAAAGGGGCCGGUGCUGGCGCUCGCAUUCAAUAAAGAGUAAUGUGGCAUUUUUCUAU
AAUAAACAUGGAUUACCUGUGUUUAAAAAAAAAAGUGUGAAUGGCGUUAGGGUAAGGGCACAGCCAGGCUGGAGUCAGUGUCU
GCCCCUGAGGUCUUUUAAGUUGAGGGCUGGGAAUGAAACCUAUAGCCUUUGUGCUGUUCUGCCUUGCCUGUGAGCUAUGUCAC
UCCCCUCCCACUCCUGACCAUAUUCCAGACACCUGCCCUAAUCCUCAGCCUGCUCACUUCACUUCUGCAUUAUAUCUCCAAGG
CGUUGGUAUAUGGAAAAAGAUGUAGGGGCUUGGAGGUGUUCUGGACAGUGGGGAGGGCUCCAGACCCAACCUGGUCACAGAAG
AGCCUCUCCCCCAUGCAUACUCAUCCACCUCCCUCCCCUAGAGCUAUUCUCCUUUGGGUUUCUUGCUGCUUCAAUUUUAUACA
ACCAUUAUUUAAAUAUUAUUAAACACAUAUUGUUCUCUAGGCACUGUGGUAGUGGGUUUUUUUGUUGUUUUUGUUUUUGAGAC
UGUCUCAAAAACUCUGUCGCCCAGGCUGACAGUGCAGUGGCACAAUCUUGGCUCACUGCAGCCUCUGCCUCCUGGGUUCAAGC
GAUUCUCGUGCAUCAGCCUCCUGAGUAACUGGAAUUAAUAGGCACGUGCCACCAUGUCCAUCUAAUUCAUAUAUAUAUAUUUU
UUUUUUCUGAGACGGAGUCUCACUGUCACACAGGCUGGAGUGCAGUGGCACGAUCUCGACUCACUGCAAGCUCCACCUCCUGG
GUUCACGCCAUUCUCCUGCCUCAGCCUCCCCAGUAGCUGGGACUACAGGCGCCCGCCACCACGCCCGGCUAAUUUUUUGUAUU
UUUAGUAGAGAUGGGGUUUCUCCGUGUUAGCCAGGAUGGUCUCGAUCUCCUGACCUCGUGAUCCGCCCGCCUUGGCCUCCCAA
AGUGCUGGGAUUACAGGCGUGAGCCACCGCGCCCGGCCGAAUUCAUCUAUUUUUAGUAGAGAUGGGGUUUCACUAUAUUGGCC
AGGCUGGUCUUGAACUCAUGACCUCAGAUGUUCACUUGUCUUGGCCUCCCAAAGUGCUGGGAUUAGAGGCGUGAGCCACCGCA
CGCGGGCCUGUGGUAAAUUGUUGAAUUUGAAGGACUCAGAGGCCCUGGUCAAUUCCAAAAUAACGUAGGCGACUUCCAUCCCC
CUCCUCCCAACCAUUUUCAGCCCAAAGCAUCUUCGCAGGGAAUGGAUGGCUGCGCGGAGGUGGGCGGUGGCUCUGGAGAGGGU
CUUUGCAGGUGUGAUUUUCUCUAGAAGGAAAUGUCUCGUCGUGGACCCAGACUGCCCCCUCCUGGUUUCAGAUGCAGAAGUGA
UACUGUAAGCCAGAGGCGGGGGCAGUAAUGCAUCGCAGCCAUUUUAGGUGAGGAUUUCCUUGGCGGUUAUUUGUUAAGUUCUU
UGGCUGGGCCCUGGGCUGGGGUAACAAUGGACAGGUUCCAGGCAUUUUUUUCAGAAAGCUUCCAGUGUAGUGGAUACAGAAAC
UUCAGGAAGGCAGGGCUGAGAAGGAUCUGAGUAAAACUCGGUCCUUCAACACCAUCCUUCAGCCCCUGGGUCAUGUUCCUUCG
AGGUCCUGGUGGGAGGUAGACAAGCCUAGCCUUGUGCUGUUCCUGUAAGGACAGGGUGGGCAUUUUCUACCAACAGAAUUCUU
GGAAUUUUCACACAGCCCAGCCUAGCCAAGUCCAGGGCUAUAGCCCAGAUACACAAGUUAAGGUCCCAGCACUGGCACCCACC
ACAGGAGCCCCCUUACCUCUAUUACCCAGAAGCUUGUAGGAGGGGUGGUCCGCAGACAAGGACCCUGCACAGGUGCGACCCUG
CUUCCCUCCUGGUCAUAACUUUCAUGUUACUAUUGCUUGGGAUAAUGUUAAGUAAAAAUAGCAGACACUGAGUUUUAAGUCUC
AAGUGGAUGAAGGCAGAGAUCGUGAUACACUUGAGUUAAAGCAGUAGGGUUCUGUCAUUUUCUAUUCCUGUUGUAAACAUUUU
CUUUAAUGUUAUUAUUUUUACCACUAAACUAACGUGGCCUGGUCACGACUUUCAUUGGUAAAGUGUGCUGUUCCUCACCCUCC
ACCGUUGCUCCUUUGGUCCACUGAUCAUAAGAGCAUUUACCUGAAGGUCGUCAGACCUCGAAUGCCAACAGGUCAACUGCAGU
GGCCUGCAGUUACCACCCAGUCUGUUCCAAUGAACAGAAUCGCUGUUGCCCCAACUCAUCUCCCUUCACCUAGGCUGUAAAUU
GAAAGUCCCACCCCUGAGCGGAACACAGGCCAUCUUGUGUGCUGUGCACCACCAGGGGGUGGGGAAGUUUCCAGACUGACUUC
CUGGCUCCAGUCAUCCUAGGAAAAGAGUUCUCCAGUCGCUCCCCACCCCCACCCCUUCCCAUUCCAGGAGUCUAUUAAGGAGG
CAAAGCAGGCCUAACGGGUAUCAAAGCAAAGGAGUGAAUGGAGACUGGGAGAGUCUUCAACCUCUCCUCUCCUUGGUAGGAGC
UGAGGCUGCAUGCCAGGUACCUUCCCUUCGAGGAAUCUAAUAAAGCUAGGUCACUGGUGUUUUCAGGUGCUUCUCAAAGGAUU
GCCGUAGGGGUAGGAUAUCAGGAUGUGGGAGCACAGGUGCCACCACAGCACUAGUGAUGGAGAGUCAUUGCCCCUAGACUUCU
GGGACAGUGAGACUGUGAGGAAAGCUGAAAUGAUACUGGGAAAGGGUGAAAGAAAGGAUGUAGGUGGAAUUUAUUUAGUAUUA
AUGUAGGUACACAUACCUUAUGGCAACAUUCCUAGCACUCUAAAUUCUAGAUUUGUAUAGUUUCUGUCAAUAUCUUUUGUAAG
CUUAAUCAAUACAGGGCAUGACAAGUAUGUGUCACAUACUUUUUUUUCCACGAAGAAAAAAAAUAAGUAGGAAUUGGGUGCUU
UGUUUAUCAAAAUUUGUAUUUCCUUUAUAAAUAAACUUUGAAAUAAAGGUUGAAAAUUAGUA

Applying the difference-based method while preserving UTR sequences and structure (−MFE=1925).

SEQ ID NO: 69 provides a design for a nucleic acid mRNA sequence trained from
Homo sapiens reference HEXA mRNA under UTR-sequence-and-structure constraints
higher expression design level

CUCACGUGGCCAGCCCCCUCCGAGAGGGGAGACCAGCGGGCCAUGACUUCUUCAAGGCUUUGGUUUAGCCUCCUGCUCGCAGC
GGCGUUUGCCGGACGGGCUACGGCCCUCUGGCCCUGGCCUCAAAACUUCCAGACAUCCGACCAGCGCUAUGUCCUUUACCCGA
ACAAUUUUCAAUUCCAGUACGAUGUCAGCUCGGCCGCACAGCCGGGCUGCUCAGUCCUCGAUGAAGCCUUCCAGCGCUAUCGU
GAUCUGCUUUUUGGUUCCGGGUCUUGGCCCCGUCCGUACCUGACCGGGAAACGGCAUACACUGGAGAAGAAUGUGUUGGUUGU
CUCUGUAGUCACACCUGGAUGUAACCAGCUUCCUACCUUGGAGUCAGUGGAAAACUACACUCUGACCAUAAAUGAUGACCAGU
GUUUACUCCUCUCGGAGACCGUGUGGGGGGCUCUACGCGGUCUGGAGACCUUUAGCCAACUGGUUUGGAAGUCUGCGGAGGGG
ACUUUCUUCAUCAAUAAGACUGAGAUUGAGGACUUUCCCCGCUUUCCUCACCGGGGCUUGCUGUUGGACACAUCUCGCCACUA
UCUGCCGCUGUCGAGCAUACUCGACACCCUUGAUGUCAUGGCGUACAAUAAAUUGAACGUGUUUCACUGGCAUUUAGUUGACG
ACCCCUCUUUUCCGUAUGAGUCCUUUACUUUUCCGGAACUCAUGCGGAAGGGGUCGUACAACCCUGUCACGCACAUUUAUACU
GCGCAGGACGUCAAGGAGGUGAUCGAGUAUGCUCGACUGCGGGGGAUCCGUGUGCUCGCGGAGUUUGAUACUCCUGGCCACAC
UCUCUCAUGGGGUCCUGGAAUCCCCGGGUUGCUCACCCCGUGUUAUUCAGGCUCGGAGCCAUCGGGCACUUUUGGCCCGGUGA
AUCCGAGCCUGAAUAACACGUACGAGUUUAUGUCCACCUUCUUCCUCGAGGUGAGUAGUGUCUUCCCUGACUUCUACCUUCAC
CUCGGGGGAGAUGAGGUGGACUUUACUUGUUGGAAGAGCAACCCGGAGAUCCAGGACUUCAUGAGAAAGAAGGGUUUUGGGGA
GGACUUCAAGCAAUUGGAGUCCUUCUACAUUCAGACCCUUCUCGACAUAGUCUCUUCCUACGGGAAGGGCUAUGUCGUGUGGC
AGGAGGUGUUUGACAACAAGGUCAAAAUUCAGCCCGAUACGAUCAUACAGGUCUGGCGAGAGGAUAUUCCAGUGAAUUAUAUG
AAGGAGCUGGAGCUGGUCACGAAAGCCGGCUUUCGGGCCCUUCUCUCUGCCCCCUGGUACCUGAACCGUAUAUCCUAUGGCCC
UGACUGGAAGGAUUUCUAUAUAGUAGAACCCCUGGCAUUUGAAGGUACUCCUGAGCAGAAGGCUCUGGUGAUUGGUGGAGAGG
CUUGCAUGUGGGGGGAAUACGUUGACAACACGAACCUUGUUCCCAGGUUGUGGCCCCGCGCAGGGGCUGUGGCUGAACGCCUC
UGGAGCAACAAGCUCACAUCGGAUUUGACUUUUGCGUACGAGCGCUUAAGUCAUUUCCGAUGUGAGCUGUUGCGCAGAGGCGU
UCAGGCACAGCCCCUGAACGUAGGCUUCUGUGAGCAGGAGUUUGAACAGACCUGAGCCCCAGGCACCGAGGAGGGUGCUGGCU
GUAGGUGAAUGGUAGUGGAGCCAGGCUUCCACUGCAUCCUGGCCAGGGGACGGAGCCCCUUGCCUUCGUGCCCCUUGCCUGCG
UGCCCCUGUGCUUGGAGAGAAAGGGGCCGGUGCUGGCGCUCGCAUUCAAUAAAGAGUAAUGUGGCAUUUUUCUAUAAUAAACA
UGGAUUACCUGUGUUUAAAAAAAAAAGUGUGAAUGGCGUUAGGGUAAGGGCACAGCCAGGCUGGAGUCAGUGUCUGCCCCUGA
GGUCUUUUAAGUUGAGGGCUGGGAAUGAAACCUAUAGCCUUUGUGCUGUUCUGCCUUGCCUGUGAGCUAUGUCACUCCCCUCC
CACUCCUGACCAUAUUCCAGACACCUGCCCUAAUCCUCAGCCUGCUCACUUCACUUCUGCAUUAUAUCUCCAAGGCGUUGGUA
UAUGGAAAAAGAUGUAGGGGCUUGGAGGUGUUCUGGACAGUGGGGAGGGCUCCAGACCCAACCUGGUCACAGAAGAGCCUCUC
CCCCAUGCAUACUCAUCCACCUCCCUCCCCUAGAGCUAUUCUCCUUUGGGUUUCUUGCUGCUUCAAUUUUAUACAACCAUUAU
UUAAAUAUUAUUAAACACAUAUUGUUCUCUAGGCACUGUGGUAGUGGGUUUUUUUGUUGUUUUUGUUUUUGAGACUGUCUCAA
AAACUCUGUCGCCCAGGCUGACAGUGCAGUGGCACAAUCUUGGCUCACUGCAGCCUCUGCCUCCUGGGUUCAAGCGAUUCUCG
UGCAUCAGCCUCCUGAGUAACUGGAAUUAAUAGGCACGUGCCACCAUGUCCAUCUAAUUCAUAUAUAUAUAUUUUUUUUUUCU
GAGACGGAGUCUCACUGUCACACAGGCUGGAGUGCAGUGGCACGAUCUCGACUCACUGCAAGCUCCACCUCCUGGGUUCACGC
CAUUCUCCUGCCUCAGCCUCCCCAGUAGCUGGGACUACAGGCGCCCGCCACCACGCCCGGCUAAUUUUUUGUAUUUUUAGUAG
AGAUGGGGUUUCUCCGUGUUAGCCAGGAUGGUCUCGAUCUCCUGACCUCGUGAUCCGCCCGCCUUGGCCUCCCAAAGUGCUGG
GAUUACAGGCGUGAGCCACCGCGCCCGGCCGAAUUCAUCUAUUUUUAGUAGAGAUGGGGUUUCACUAUAUUGGCCAGGCUGGU
CUUGAACUCAUGACCUCAGAUGUUCACUUGUCUUGGCCUCCCAAAGUGCUGGGAUUAGAGGCGUGAGCCACCGCACGCGGGCC
UGUGGUAAAUUGUUGAAUUUGAAGGACUCAGAGGCCCUGGUCAAUUCCAAAAUAACGUAGGCGACUUCCAUCCCCCUCCUCCC
AACCAUUUUCAGCCCAAAGCAUCUUCGCAGGGAAUGGAUGGCUGCGCGGAGGUGGGCGGUGGCUCUGGAGAGGGUCUUUGCAG
GUGUGAUUUUCUCUAGAAGGAAAUGUCUCGUCGUGGACCCAGACUGCCCCCUCCUGGUUUCAGAUGCAGAAGUGAUACUGUAA
GCCAGAGGCGGGGGCAGUAAUGCAUCGCAGCCAUUUUAGGUGAGGAUUUCCUUGGCGGUUAUUUGUUAAGUUCUUUGGCUGGG
CCCUGGGCUGGGGUAACAAUGGACAGGUUCCAGGCAUUUUUUUCAGAAAGCUUCCAGUGUAGUGGAUACAGAAACUUCAGGAA
GGCAGGGCUGAGAAGGAUCUGAGUAAAACUCGGUCCUUCAACACCAUCCUUCAGCCCCUGGGUCAUGUUCCUUCGAGGUCCUG
GUGGGAGGUAGACAAGCCUAGCCUUGUGCUGUUCCUGUAAGGACAGGGUGGGCAUUUUCUACCAACAGAAUUCUUGGAAUUUU
CACACAGCCCAGCCUAGCCAAGUCCAGGGCUAUAGCCCAGAUACACAAGUUAAGGUCCCAGCACUGGCACCCACCACAGGAGC
CCCCUUACCUCUAUUACCCAGAAGCUUGUAGGAGGGGUGGUCCGCAGACAAGGACCCUGCACAGGUGCGACCCUGCUUCCCUC
CUGGUCAUAACUUUCAUGUUACUAUUGCUUGGGAUAAUGUUAAGUAAAAAUAGCAGACACUGAGUUUUAAGUCUCAAGUGGAU
GAAGGCAGAGAUCGUGAUACACUUGAGUUAAAGCAGUAGGGUUCUGUCAUUUUCUAUUCCUGUUGUAAACAUUUUCUUUAAUG
UUAUUAUUUUUACCACUAAACUAACGUGGCCUGGUCACGACUUUCAUUGGUAAAGUGUGCUGUUCCUCACCCUCCACCGUUGC
UCCUUUGGUCCACUGAUCAUAAGAGCAUUUACCUGAAGGUCGUCAGACCUCGAAUGCCAACAGGUCAACUGCAGUGGCCUGCA
GUUACCACCCAGUCUGUUCCAAUGAACAGAAUCGCUGUUGCCCCAACUCAUCUCCCUUCACCUAGGCUGUAAAUUGAAAGUCC
CACCCCUGAGCGGAACACAGGCCAUCUUGUGUGCUGUGCACCACCAGGGGGUGGGGAAGUUUCCAGACUGACUUCCUGGCUCC
AGUCAUCCUAGGAAAAGAGUUCUCCAGUCGCUCCCCACCCCCACCCCUUCCCAUUCCAGGAGUCUAUUAAGGAGGCAAAGCAG
GCCUAACGGGUAUCAAAGCAAAGGAGUGAAUGGAGACUGGGAGAGUCUUCAACCUCUCCUCUCCUUGGUAGGAGCUGAGGCUG
CAUGCCAGGUACCUUCCCUUCGAGGAAUCUAAUAAAGCUAGGUCACUGGUGUUUUCAGGUGCUUCUCAAAGGAUUGCCGUAGG
GGUAGGAUAUCAGGAUGUGGGAGCACAGGUGCCACCACAGCACUAGUGAUGGAGAGUCAUUGCCCCUAGACUUCUGGGACAGU
GAGACUGUGAGGAAAGCUGAAAUGAUACUGGGAAAGGGUGAAAGAAAGGAUGUAGGUGGAAUUUAUUUAGUAUUAAUGUAGGU
ACACAUACCUUAUGGCAACAUUCCUAGCACUCUAAAUUCUAGAUUUGUAUAGUUUCUGUCAAUAUCUUUUGUAAGCUUAAUCA
AUACAGGGCAUGACAAGUAUGUGUCACAUACUUUUUUUUCCACGAAGAAAAAAAAUAAGUAGGAAUUGGGUGCUUUGUUUAUC
AAAAUUUGUAUUUCCUUUAUAAAUAAACUUUGAAAUAAAGGUUGAAAAUUAGUA

### K. Example 11: Gene Training as a Suppressive Approach to Mitigate, Treat, or Prevent Genetic Disease

In this example we focus on suppressing a V50M mutated TTR to help delay, slow, reduce, mute, or prevent autosomal dominant Familial Amyloid Polyneuropathy and/or Amyloidogenic Transthyretin Amyloidosis.

In this example, we use the forward sequences and reverse complement sequences of Genome GCF_000001405.40_GRCh38.p14 genomic.fna as well as the sequences of Transcriptome rna.fna (mRNA, ncRNA, and miscellaneous RNA) from reference GCF_000001405.40 as a non-pathogenic reference set, retrieved 2024 Apr. 3. In this example, the reference set was chosen to illustrate an example design of MS-ASOs, whereas in practice we may chose a larger, smaller, or different reference set, or simply the reference set from the intended treatment recipient, depending on the tailored or personalized application and target organism(s), both for design of MS-ASOs and/or pareto-specific ASOs, but also to assess or determine applicability of already designed pareto-specific ASOs and/or MS-ASOs to additional target organism(s) or applications. For this example, for illustration only, we use a single genome and single transcriptome in the reference set to avoid obscuring the disclosure and to illustrate an approach to averting inadvertent and/or negative side effects of binding to off-target DNA. However, one skilled in the art shall understand that the above procedures can be applied when the reference set represents many, many non-pathogenic genomes and/or transcriptomes, for example via a labeled directed graph. Furthermore, inclusion of a genome in the reference set in the examples of this disclosure is intended to illustrate averting inadvertent and/or negative side effects of binding to off-target DNA.

A genome need not be included in the reference set at all if the stakeholders are comfortable with the risk of inadvertent and/or negative side effects of an ASO potentially binding to off-target DNA or off-target primary transcripts. Similarly, the transcript sequences of appropriate and relevant non-pathogenic primary transcriptome(s) might be substituted for the genome(s) if the stakeholders are comfortable with the risk of inadvertent and/or negative side effects of an ASO potentially binding to off-target DNA.

In this example, a V50M mutated TTR with a single nucleotide polymorphism, shown below, is used as the (m) RNA sequence target to which each ASO is designed for target-specificity with respect to the selected reference set.

However, one skilled in the art shall recognize and understand that the methods of this disclosure for designing MS-ASOs work for transcripts that may contain multiple nucleotide polymorphisms, insertions, and/or deletions, as well as alternate sequences encoding protein. For the purposes of this simplified example, the V50M mutated TTR is considered pathogenic, whereas the unmutated TTR from the reference set is considered non-pathogenic.

A V50M mutated TTR CDS RNA (mutation site highlighted in bold with wavy underline):

SEQ ID NO: 70 provides a nucleic acid sequence scenario with CDS encoding a variant of a
putatively pathogenic V50M mutant TTR protein of Homo sapiens
AUGGCUUCUCAUCGUCUGCUCCUCCUCUGCCUUGCUGGACUGGUAUUUGUGUCUGAGGCUGGCCCUACGGGCACCGGUGAAUC

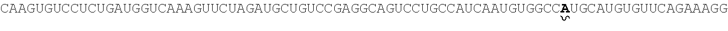

CUGCUGAUGACACCUGGGAGCCAUUUGCCUCUGGGAAAACCAGUGAGUCUGGAGAGCUGCAUGGGCUCACAACUGAGGAGGAA
UUUGUAGAAGGGAUAUACAAAGUGGAAAUAGACACCAAAUCUUACUGGAAGGCACUUGGCAUCUCCCCAUUCCAUGAGCAUGC
AGAGGUGGUAUUCACAGCCAACGACUCCGGCCCCCGCCGCUACACCAUUGCCGCCCUGCUGAGCCCCUACUCCUAUUCCACCA
CGGCUGUCGUCACCAAUCCCAAGGAAUGA

Reverse Complement of the V50M mutated TTR CDS RNA (mutation site highlighted in bold with wavy underline):

SEQ ID NO: 71 provides the reverse complement of a nucleic acid sequence scenario with CDS
encoding a variant of a putatively pathogenic V50M mutant TTR protein of Homo sapiens
UCAUUCCUUGGGAUUGGUGACGACAGCCGUGGUGGAAUAGGAGUAGGGGCUCAGCAGGGCGGCAAUGGUGUAGCGGCGGGGGC
CGGAGUCGUUGGCUGUGAAUACCACCUCUGCAUGCUCAUGGAAUGGGGAGAUGCCAAGUGCCUUCCAGUAAGAUUUGGUGUCU
AUUUCCACUUUGUAUAUCCCUUCUACAAAUUCCUCCUCAGUUGUGAGCCCAUGCAGCUCUCCAGACUCACUGGUUUUCCCAGA

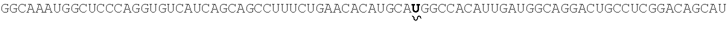

CUAGAACUUUGACCAUCAGAGGACACUUGGAUUCACCGGUGCCCGUAGGGCCAGCCUCAGACACAAAUACCAGUCCAGCAAGG
CAGAGGAGGAGCAGACGAUGAGAAGCCAU

Given the mutated transcript and using the procedures above, the following are some identified doubly-minimal signatures with the presumably pathogenic nucleotide mutation highlighted in bold with wavy underline.

Examples of doubly-minimal signatures for the above V50M mutated TTR with respect to the example reference set, with gene-variation site of interest in bold with wavy underline:

SEQ ID NO: 72 provides a polynucleotide sequence of a doubly-minimal signature of a
simulated V50M mutant TTR of Homo sapiens

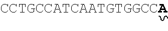

SEQ ID NO: 73 provides a polynucleotide sequence of a doubly-minimal signature of a
simulated V50M mutant TTR of Homo sapiens

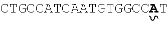

SEQ ID NO: 74 provides a polynucleotide sequence of a doubly-minimal signature of a
simulated V50M mutant TTR of Homo sapiens

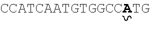

SEQ ID NO: 75 provides a polynucleotide sequence of a doubly-minimal signature of a
simulated V50M mutant TTR of Homo sapiens

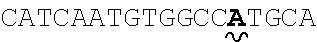

SEQ ID NO: 76 provides a polynucleotide sequence of a doubly-minimal signature of a
simulated V50M mutant TTR of Homo sapiens

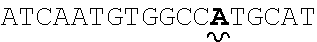

SEQ ID NO: 77 provides a polynucleotide sequence of a doubly-minimal signature of a
simulated V50M mutant TTR of Homo sapiens

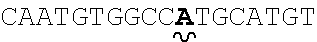

SEQ ID NO: 78 provides a polynucleotide sequence of a doubly-minimal signature of a
simulated V50M mutant TTR of Homo sapiens

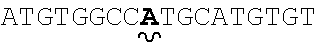

SEQ ID NO: 79 provides a polynucleotide sequence of a doubly-minimal signature of a
simulated V50M mutant TTR of Homo sapiens

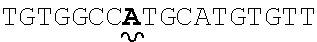

SEQ ID NO: 80 provides a polynucleotide sequence of a doubly-minimal signature of a
simulated V50M mutant TTR of Homo sapiens

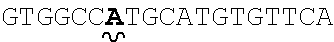

SEQ ID NO: 81 provides a polynucleotide sequence of a doubly-minimal signature of a
simulated V50M mutant TTR of Homo sapiens

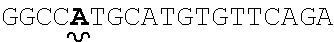

SEQ ID NO: 82 provides a polynucleotide sequence of a doubly-minimal signature of a
simulated V50M mutant TTR of Homo sapiens

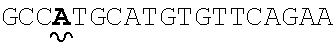

SEQ ID NO: 83 provides a polynucleotide sequence of a doubly-minimal signature of a
simulated V50M mutant TTR of Homo sapiens

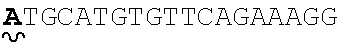

Given the mutated transcript and using the procedures above, the following are some identified maximally-specific ASOs with the gene-variation site-binding nucleotide highlighted in bold with wavy underline that presumably binds the pathogenic mutation.

Examples of Maximally-Specific Antisense Oligonucleotides for the above V50M mutated TTR with respect to the example reference set:

SEQ ID NO: 84 provides a sequence for design of an antisense oligonucleotide corresponding to
a doubly-minimal signature of a V50M mutated TTR of Homo sapiens

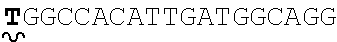

SEQ ID NO: 85 provides a sequence for design of an antisense oligonucleotide corresponding to
a doubly-minimal signature of a simulated V50M mutated TTR of Homo sapiens

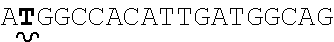

SEQ ID NO: 1 provides a sequence for design of an antisense oligonucleotide corresponding to
a doubly-minimal signature of a simulated V50M mutated TTR of Homo sapiens

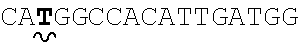

SEQ ID NO: 2 provides a sequence for design of an antisense oligonucleotide corresponding to
a doubly-minimal signature of a simulated V50M mutated TTR of Homo sapiens

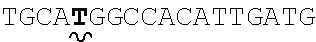

SEQ ID NO: 3 provides a sequence for design of an antisense oligonucleotide corresponding to
a doubly-minimal signature of a simulated V50M mutated TTR of Homo sapiens

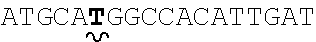

SEQ ID NO: 4 provides a sequence for design of an antisense oligonucleotide corresponding to
a doubly-minimal signature of a simulated V50M mutated TTR of Homo sapiens

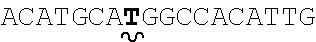

SEQ ID NO: 5 provides a sequence for design of an antisense oligonucleotide corresponding to
a doubly-minimal signature of a simulated V50M mutated TTR of Homo sapiens

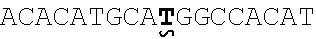

SEQ ID NO: 6 provides a sequence for design of an antisense oligonucleotide corresponding to
a doubly-minimal signature of a simulated V50M mutated TTR of Homo sapiens

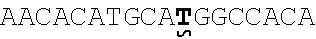

SEQ ID NO: 7 provides a sequence for design of an antisense oligonucleotide corresponding to
a doubly-minimal signature of a simulated V50M mutated TTR of Homo sapiens

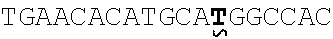

SEQ ID NO: 8 provides a sequence for design of an antisense oligonucleotide corresponding to
a doubly-minimal signature of a simulated V50M mutated TTR of Homo sapiens

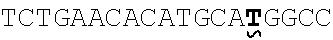

SEQ ID NO: 9 provides a sequence for design of an antisense oligonucleotide corresponding to
a doubly-minimal signature of a simulated V50M mutated TTR of Homo sapiens

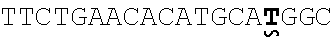

SEQ ID NO: 95 provides a sequence for design of an antisense oligonucleotide corresponding to
a doubly-minimal signature of a simulated V50M mutated TTR of Homo sapiens

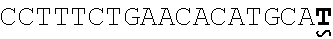

Examples of Antisense Oligonucleotides that are not (and have no extension that is a) MS-ASO for the above V50M mutated TTR with respect to the example reference set with gene-variation-binding site in bold with wavy underline:

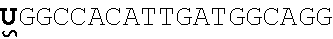

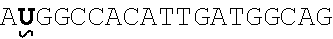

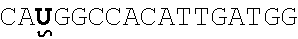

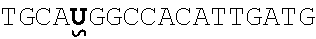

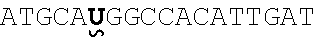

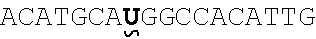

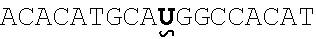

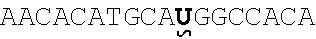

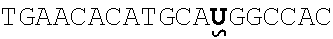

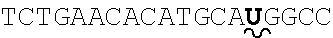

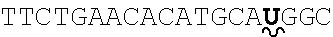

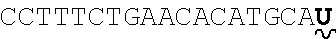

The following are pathogenic minimal signatures of DNA that identify the pathogenic variant of this example with pathogenic variation in bold with wavy underline.

+Example Pathogenic Minimal Signatures with respect to the example reference set DNA:

SEQ ID NO: 96 provides a sequence of a doubly-minimal DNA signature of a simulated V50M
mutant TTR of Homo sapiens

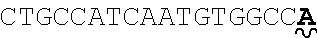

SEQ ID NO: 97 provides a polynucleotide sequence of a doubly-minimal DNA signature of a
simulated V50M mutant TTR of Homo sapiens

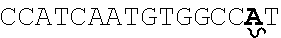

SEQ ID NO: 10 provides a polynucleotide sequence of a doubly-minimal DNA signature of a
simulated V50M mutant TTR of Homo sapiens

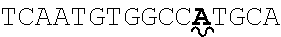

SEQ ID NO: 11 provides a polynucleotide sequence of a doubly-minimal DNA signature of a
simulated V50M mutant TTR of Homo sapiens

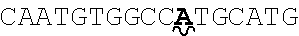

SEQ ID NO: 12 provides a polynucleotide sequence of a doubly-minimal DNA signature of a
simulated V50M mutant TTR of Homo sapiens

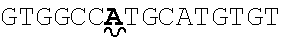

SEQ ID NO: 13 provides a polynucleotide sequence of a doubly-minimal DNA signature of a
simulated V50M mutant TTR of Homo sapiens

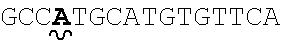

SEQ ID NO: 14 provides a polynucleotide sequence of a doubly-minimal DNA signature of a
simulated V50M mutant TTR of Homo sapiens

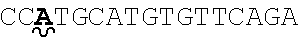

SEQ ID NO: 15 provides a polynucleotide sequence of a doubly-minimal DNA signature of a
simulated V50M mutant TTR of Homo sapiens

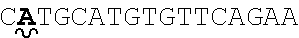

SEQ ID NO: 16 provides a polynucleotide sequence of a doubly-minimal DNA signature of a
simulated V50M mutant TTR of Homo sapiens

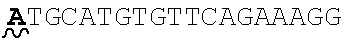

SEQ ID NO: 17 provides a polynucleotide sequence of a doubly-minimal DNA signature of a
simulated V50M mutant TTR of Homo sapiens

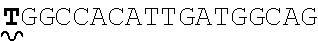

SEQ ID NO: 18 provides a polynucleotide sequence of a doubly-minimal DNA signature of a
simulated V50M mutant TTR of Homo sapiens

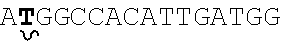

SEQ ID NO: 19 provides a polynucleotide sequence of a doubly-minimal DNA signature of a
simulated V50M mutant TTR of Homo sapiens

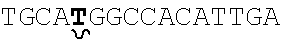

SEQ ID NO: 20 provides a polynucleotide sequence of a doubly-minimal DNA signature of a
simulated V50M mutant TTR of Homo sapiens

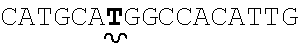

SEQ ID NO: 21 provides a polynucleotide sequence of a doubly-minimal DNA signature of a
simulated V50M mutant TTR of Homo sapiens

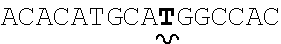

SEQ ID NO: 22 provides a polynucleotide sequence of a doubly-minimal DNA signature of a
simulated V50M mutant TTR of Homo sapiens

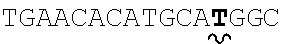

SEQ ID NO: 23 provides a polynucleotide sequence of a doubly-minimal DNA signature of a
simulated V50M mutant TTR of Homo sapiens

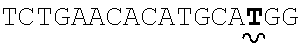

SEQ ID NO: 24 provides a polynucleotide sequence of a doubly-minimal DNA signature of a
simulated V50M mutant TTR of Homo sapiens

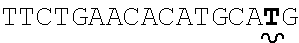

SEQ ID NO: 113 provides a polynucleotide sequence of a doubly-minimal DNA signature of a
simulated V50M mutant TTR of Homo sapiens

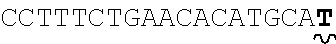

In the above V50M TTR variant example, the provided pathogenic minimal signatures can be used to detect the pathogenic single nucleotide polymorphism shown, that is said to indicate predisposition for the associated pathogenic phenotype(s).

The following is the non-pathogenic TTR CDS RNA variant from the above-mentioned reference set with the mutation site underlined.

SEQ ID NO: 114 provides the nucleic acid
sequence of the TTR CDS encoding a variant
of TTR protein from Homo sapiens

AUGGCUUCUCAUCGUCUGCUCCUCCUCUGCCUUGCUGGACUGGUAUUUG
UGUCUGAGGCUGGCCCUACGGGCACCGGUGAAUCCAAGUGUCCUCUGAU
GGUCAAAGUUCUAGAUGCUGUCCGAGGCAGUCCUGCCAUCAAUGUGGCC
GUGCAUGUGUUCAGAAAGGCUGCUGAUGACACCUGGGAGCCAUUUGCCU
CUGGGAAAACCAGUGAGUCUGGAGAGCUGCAUGGGCUCACAACUGAGGA
GGAAUUUGUAGAAGGGAUAUACAAAGUGGAAAUAGACACCAAAUCUUAC
UGGAAGGCACUUGGCAUCUCCCCAUUCCAUGAGCAUGCAGAGGUGGUAU
UCACAGCCAACGACUCCGGCCCCCGCCGCUACACCAUUGCCGCCCUGCU
GAGCCCCUACUCCUAUUCCACCACGGCUGUCGUCACCAAUCCCAAGGAA
UGA

The following are some example non-pathogenic on-site locus signatures (of non-pathogenic reference DNA) with respect to the reference sets, when the pathogenic set is defined as the non-pathogenic reference set together with the example V50M mutated TTR polynucleotide sequences. The site of the gene-variation of interest is highlighted in bold with wavy underline:

SEQ ID NO: 115 provides a polynucleotide sequence of a minimal on-site locus signature of a
putatively non-pathogenic TTR of Homo sapiens

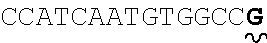

SEQ ID NO: 116 provides a polynucleotide sequence of a minimal on-site locus signature of a
putatively non-pathogenic TTR of Homo sapiens

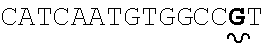

SEQ ID NO: 117 provides a polynucleotide sequence of a minimal on-site locus signature of a
putatively non-pathogenic TTR of Homo sapiens

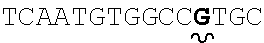

SEQ ID NO: 118 provides a polynucleotide sequence of a minimal on-site locus signature of a
putatively non-pathogenic TTR of Homo sapiens

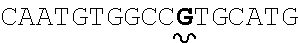

SEQ ID NO: 119 provides a polynucleotide sequence of a minimal on-site locus signature of a
putatively non-pathogenic TTR of Homo sapiens

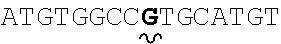

SEQ ID NO: 120 provides a polynucleotide sequence of a minimal on-site locus signature of a
putatively non-pathogenic TTR of Homo sapiens

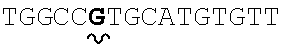

SEQ ID NO: 25 provides a polynucleotide sequence of a minimal on-site locus signature of a
putatively non-pathogenic TTR of Homo sapiens

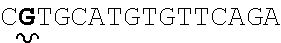

SEQ ID NO: 26 provides a polynucleotide sequence of a minimal on-site locus signature of a
putatively non-pathogenic TTR of Homo sapiens

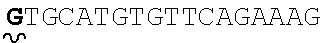

SEQ ID NO: 27 provides a polynucleotide sequence of a minimal on-site locus signature of a
putatively non-pathogenic TTR of Homo sapiens

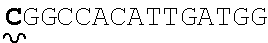

SEQ ID NO: 28 provides a polynucleotide sequence of a minimal on-site locus signature of a
putatively non-pathogenic TTR of Homo sapiens

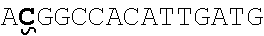

SEQ ID NO: 29 provides a polynucleotide sequence of a minimal on-site locus signature of a
putatively non-pathogenic TTR of Homo sapiens

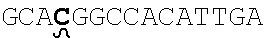

SEQ ID NO: 30 provides a polynucleotide sequence of a minimal on-site locus signature of a
putatively non-pathogenic TTR of Homo sapiens

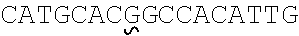

SEQ ID NO: 31 provides a polynucleotide sequence of a minimal on-site locus signature of a
putatively non-pathogenic TTR of Homo sapiens

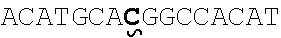

SEQ ID NO: 32 provides a polynucleotide sequence of a minimal on-site locus signature of a
putatively non-pathogenic TTR of Homo sapiens

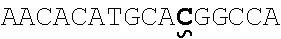

SEQ ID NO: 33 provides a polynucleotide sequence of a minimal on-site locus signature of a
putatively non-pathogenic TTR of Homo sapiens

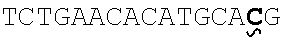

SEQ ID NO: 130 provides a polynucleotide sequence of a minimal on-site locus signature of a
putatively non-pathogenic TTR of Homo sapiens

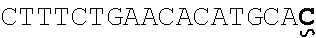

A marker designed to detect a pathogenic signature and a non-pathogenic signature, such as the examples above may be used to help detect a pathogenic predisposition with PCR or digital PCR.

A marker designed to detect a pathogenic signature such as the examples above may be used together with a control gene sequence to help detect a pathogenic predisposition. Signatures including, but not limited to, flanking locus signatures can be used as a control gene sequence, to be used for example with digital PCR. As examples, a couple example flanking locus signatures with respect to the pathogenic and non-pathogenic reference sets for the TTR DNA are provided below.

SEQ ID NO: 131 provides a polynucleotide sequence
of a locus signature flanking the V50M variant
site of TTR of Homo sapiens

TGCATGTGTTCAGAAAGG

SEQ ID NO: 132 provides a polynucleotide sequence
of a locus signature flanking the V50M variant
site of TTR of Homo sapiens

CCTTTCTGAACACATGCA

When an individual's genetics are used as the reference set and are in the heterozygous condition, the respectively identified ASOs may help delay, slow, reduce, mute, and/or prevent the medical conditions of Familial Amyloid Polyneuropathy and Amyloidogenic Transthyretin Amyloidosis. When an individual's genetics are used as the reference set and are in the homozygous for the pathogenic variant, the respectively identified ASOs may help prevent Familial Amyloid Polyneuropathy and help prevent Amyloidogenic Transthyretin Amyloidosis better when delivered properly in combination with supplementation of gene trained TTR mRNA variant from the reference set, such as the one given below.

TTR mRNA with CDS[0-1] Design Level @ 100% Expression

SEQ ID NO: 133 provides a design for a mRNA
sequence with CDS trained to a 100% expression
design level encoding a variant of a TTR
protein from Homo sapiens

ACAGAAGUCCACUCAUUCUUGGCAGGAUGGCCUCCCAUCGCUUGCUCCU
CCUGUGCCUGGCGGGCCUGGUCUUCGUAUCGGAGGCCGGGCCCACAGGC
ACAGGAGAGAGCAAGUGUCCCCUGAUGGUGAAGGUGCUCGACGCGGUUC
GCGGCAGCCCUGCGAUAAACGUGGCAGUCCACGUUUUUCGCAAGGCUGC
CGACGAUACGUGGGAGCCCUUCGCCUCAGGGAAAACCUCCGAAAGUGGU
GAGCUCCACGGGCUCACCACUGAGGAGGAGUUCGUGGAAGGGAUCUACA
AAGUAGAAAUUGAUACCAAGAGCUAUUGGAAAGCUCUUGGUAUCAGUCC
CUUCCACGAACAUGCGGAGGUCGUUUUCACUGCUAAUGAUUCGGGACCA
CGGCGGUAUACUAUAGCUGCGCUGCUAUCGCCGUACAGCUAUAGUACUA
CCGCCGUGGUCACGAAUCCUAAGGAGUGAGGGACUUCUCCUCCAGUGGA
CCUGAAGGACGAGGGAUGGGAUUUCAUGUAACCAAGAGUAUUCCAUUUU
UACUAAAGCAGUGUUUUCACCUCAUAUGCUAUGUUAGAAGUCCAGGCAG
AGACAAUAAAACAUUCCUGUGAAAGGCA

Here, we importantly note that the above antisense oligonucleotides that are not MS-ASOs, May still be applied when the ASO is a locus signature and a pathogenicity-specific supplemental polynucleotide is properly designed such that the PSRC of the ASO does *not* accept a subsequence of the supplemental polynucleotide nor RNA derived therefrom, if the potential risks are acceptable to the stakeholders.

The following is a TTR protein sequence:

SEQ ID NO: 134 provides the amino acid
sequence of an TTR protein of Homo sapiens

MASHRLLLLCLAGLVFVSEAGPTGTGESKCPLMVKVLDAVRGSPAINVA
VHVFRKAADDTWEPFASGKTSESGELHGLTTEEEFVEGIYKVEIDTKSY
WKALGISPFHEHAEVVFTANDSGPRRYTIAALLSPYSYSTTAVVTNPKE

UTR-sequence-and-structure-preserving mRNA with differences from reference underlined and region targeted by a selected ASO in bold.

UTR-Sequence-and-Structure-Preserving TTR mRNA:

UTR-sequence-and-structure-preserving TTR mRNA:
SEQ ID NO: 135 provides a design for a mRNA sequence trained from Homo sapiens reference
TTR mRNA under UTR-sequence-and-structure constraints to a higher expression design level
ACAGAAGUCCACUCAUUCUUGGCAGGAUGGCCUCCCAUCGCUUGCUCCUCCUGUGCCUUGCGGGCCUGGUCUUCGUAUCGGAG
GCCGGGCCCACAGGCACAGGAGAGAGCAAGUGUCCCCUGAUGGUGAAGGUGCUCGACGCGGUCCGAGGCAGUCCUGCCAUAA
ACGUGGCCGUCCAUGUGUUCAGAAAGGCUGCUGAUGAUACGUGGGAGCCAUUCGCCUCUGGGAAAACCUCCGAAAGUGGUGA
GCUCCACGGGCUCACCACUGAGGAGGAGUUCGUAGAAGGGAUCUACAAAGUGGAAAUUGACACCAAAAGCUAUUGGAAAGCUC
UUGGCAUCAGUCCAUUCCACGAACAUGCGGAGGUCGUUUUCACUGCUAAUGAUUCGGGACCACGGCGGUAUACUAUAGCUGCG
CUGCUAUCGCCGUACAGCUAUAGUACUACCGCCGUGGUCACGAAUCCUAAGGAAUGAGGGACUUCUCCUCCAGUGGACCUGAA
GGACGAGGGAUGGGAUUUCAUGUAACCAAGAGUAUUCCAUUUUUACUAAAGCAGUGUUUUCACCUCAUAUGCUAUGUUAGAAG
UCCAGGCAGAGACAAUAAAACAUUCCUGUGAAAGGCA

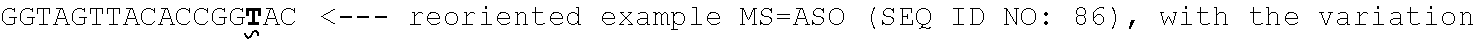

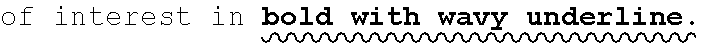

CCAUAAACGUGGCCGUC <--- targeted region of the UTR-sequence-and-structure-preserving
mRNA (SEQ ID NO: 135) already fortified against the example MS-ASO, underlining
indicating unpaired sites.

### L. Example 12: Experimental Gene Training for Hard-to-Treat Disease

In this example we focus on supplementation of HBB mRNA, while retaining secondary structure of UTR regions to help reduce, mute, or prevent development or symptoms of Sickle Cell disease. Sickle Cell disease involving HBB mRNA is an autosomal recessive disease, wherein restoration of non-pathogenic HBB may restore the structural integrity of blood cells that otherwise form the Sickle shape.

### Sickle Cell-Experimental Treatment Approach

HBB Protein Sequence:

SEQ ID NO: 136 provides the amino aci
dsequence of an HBB protein of Homo sapiens

MVHLTPEEKSAVTALWGKVNVDEVGGEALGRLLVVYPWTQRFFESFGDL
STPDAVMGNPKVKAHGKKVLGAFSDGLAHLDNLKGTFATLSELHCDKLH
VDPENFRLLGNVLVCVLAHHFGKEFTPPVQAAYQKVVAGVANALAHKYH

HBB Reference mRNA:

SEQ ID NO: 137 provides the mRNA sequence
encoding an HBB protein from Homo sapiens

ACAUUUGCUUCUGACACAACUGUGUUCACUAGCAACCUCAAACAGACAC
CAUGGUGCAUCUGACUCCUGAGGAGAAGUCUGCCGUUACUGCCCUGUGG
GGCAAGGUGAACGUGGAUGAAGUUGGUGGUGAGGCCCUGGGCAGGCUGC
UGGUGGUCUACCCUUGGACCCAGAGGUUCUUUGAGUCCUUUGGGGAUCU
GUCCACUCCUGAUGCUGUUAUGGGCAACCCUAAGGUGAAGGCUCAUGGC
AAGAAAGUGCUCGGUGCCUUUAGUGAUGGCCUGGCUCACCUGGACAACC
UCAAGGGCACCUUUGCCACACUGAGUGAGCUGCACUGUGACAAGCUGCA
CGUGGAUCCUGAGAACUUCAGGCUCCUGGGCAACGUGCUGGUCUGUGUG
CUGGCCCAUCACUUUGGCAAAGAAUUCACCCCACCAGUGCAGGCUGCCU
AUCAGAAAGUGGUGGCUGGUGUGGCUAAUGCCCUGGCCCACAAGUAUCA
CUAAGCUCGCUUUCUUGCUGUCCAAUUUCUAUUAAAGGUUCCUUUGUUC
CCUAAGUCCAACUACUAAACUGGGGGAUAUUAUGAAGGGCCUUGAGCAU
CUGGAUUCUGCCUAAUAAAAAACAUUUAUUUUCAUUGCAA

A UTR-sequence-preserving HBB mRNA with lower MFE(CR(mRNA)) than reference:

SEQ ID NO: 138 provides a design for a mRNA
sequence trained under UTR-sequence constraints
from Homo sapiens reference HBB mRNA to
a higher expression design level

ACAUUUGCUUCUGACACAACUGUGUUCACUAGCAACCUCAAACAGACAC
CAUGGUUCACCUUACCCCAGAGGAGAAGUCAGCAAAGUCAGCAGUGACU
GCCCUCUGGGGUAAGGUGAACGUGGACGAGGUCGGGGGGGAGGCCCUCG
GGCGCCUUCUUGUCGUGUAUCCUUGGACUCAGAGAUUCUUUGAGUCCUU
UGGGGAUUUGUCCACACCGGACGCGGUGAUGGGCAACCCCAAAGUCAAG
GCGCACGGCAAGAAGGUGCUCGGGGCCUUCUCCGACGGCCUCGCCCACC
UUGAUAACUUGAAGGGCACGUUCGCCACCCUCAGCGAGCUCCAUUGUGA
UAAGCUGCACGUGGACCCGGAGAAUUUUAGACUCCUUGGCAAUGUUUUG
GUGUGCGUACUCGCACACCAUUUUGGCAAGGAGUUUACUCCGCCGGUCC
AGGCAGCUUAUCAGAAGGUGGUCGCUGGGGUGGCGAACGCUCUUGCCCA
CAAGUAUCAUUAAGCUCGCUUUCUUGCUGUCCAAUUUCUAUUAAAGGUU
CCUUUGUUCCCUAAGUCCAACUACUAAACUGGGGGAUAUUAUGAAGGGC
CUUGAGCAUCUGGAUUCUGCCUAAUAAAAAACAUUUAUUUUCAUUGCAA

A UTR-sequence-and-structure-preserving HBB mRNA with lower MFE(CR(mRNA)) than reference with underlining indicating differences with reference mRNA:

SEQ ID NO: 139 provides a design for a mRNA
sequence trained under UTR-sequence-and-structure
constraints from Homo sapiens reference
HBB mRNA to a higher expression design level

ACAUUUGCUUCUGACACAACUGUGUUCACUAGCAACCUCAAACAGACAC
CAUGGUGCAUCUUACUCCAGAGGAGAAGUCUGCAGUUACUGCCCUGUGG
GGCAAGGUGAACGUGGACGAAGUUGGUGGUGAGGCCCUCGGGAGGCUGC
UGGUGGUGUAUCCUUGGACUCAGAGAUUCUUUGAGUCCUUUGGGGAUUU
GUCCACACCGGACGCGGUGAUGGGCAACCCCAAAGUCAAGGCGCACGGC
AAGAAGGUGCUCGGUGCCUUCUCCGACGGCCUGGCCCACCUUGAUAACC
UCAAGGGCACCUUUGCCACACUGAGUGAGCUGCACUGUGACAAGCUGCA
CGUGGAUCCUGAGAACUUCAGGCUCCUGGGCAACGUGCUGGUCUGUGUG
CUGGCCCAUCACUUUGGCAAAGAAUUCACCCCACCAGUGCAGGCUGCCU
AUCAGAAAGUGGUGGCUGGUGUGGCUAAUGCCCUGGCCCACAAGUAUCA
CUAAGCUCGCUUUCUUGCUGUCCAAUUUCUAUUAAAGGUUCCUUUGUUC
CCUAAGUCCAACUACUAAACUGGGGGAUAUUAUGAAGGGCCUUGAGCAU
CUGGAUUCUGCCUAAUAAAAAACAUUUAUUUUCAUUGCAA

### M. Example 13: Experimental Gene Training for Cancer Prevention

In this example, we describe a possible experimental multi-modal cancer prevention treatment of a R337H mutation in the TP53 gene. The R337H mutation is said to cause Li-Fraumeni syndrome, an autosomal dominant syndrome that causes cancer [Gencel-Augusto et al.]. It is also said that homozygous and heterozygous knockouts can also cause cancer [van Boxtel et al.]. It is also said that over-supplementation of TP53 may cause premature aging [Tyner et al.]. Because the autosomal dominant syndrome appears to at least in part be caused by haploinsufficiency (heterozygous knockout, i.e. too little p53, causes cancer) [van Boxtel et al.], and it is said that p53 tetramerization domain mutants (mutant amino acid residues between 311-367) interferes with p53's ability to form functional tetramers [Gencel-Augusto et al], and too much p53 may cause premature aging [Tyner et al.], one putative approach is to reduce or eliminate the formation of the heterotetromers by designing and introducing an ASO that reduces production of the mutant p53 while simultaneously supplementing non-mutated TP53 transcripts in moderation.

In line with this approach, we provide an example suppressive ASO sequence, an example designed supplemental mRNA, and an example fortification intended to reduce the potential interaction of the designed supplemental mRNA with the suppressive ASO.

TP53 reference mRNA sequence:

SEQ ID NO: 140 provides the TP53 mRNA sequence encoding a variant of a
p53 protein from Homo sapiens

AUUCGUGAGGGGUUUGUAAUGCAGGGCUGAGGAGUGUCCGAAGAGAAUGGGCAGGUGAGCGGUGAGACAGUUGUUCUUCCAGA
AGCUUUGCAGUGAAAGGAAUCAAAGAAAUGGAGCCGUGUAUCAGCAGCCAGACUGCCUUCCGGGUCACUGCCAUGGAGGAGCC
GCAGUCAGAUCCUAGCGUCGAGCCCCCUCUGAGUCAGGAAACAUUUUCAGACCUAUGGAAACUACUUCCUGAAAACAACGUUC
UGUCCCCCUUGCCGUCCCAAGCAAUGGAUGAUUUGAUGCUGUCCCCGGACGAUAUUGAACAAUGGUUCACUGAAGACCCAGGU
CCAGAUGAAGCUCCCAGAAUGCCAGAGGCUGCUCCCCCCGUGGCCCCUGCACCAGCAGCUCCUACACCGGCGGCCCCUGCACC
AGCCCCCUCCUGGCCCCUGUCAUCUUCUGUCCCUUCCCAGAAAACCUACCAGGGCAGCUACGGUUUCCGUCUGGGCUUCUUGC
AUUCUGGGACAGCCAAGUCUGUGACUUGCACGUACUCCCCUGCCCUCAACAAGAUGUUUUGCCAACUGGCCAAGACCUGCCCU
GUGCAGCUGUGGGUUGAUUCCACACCCCCGCCCGGCACCCGCGUCCGCGCCAUGGCCAUCUACAAGCAGUCACAGCACAUGAC
GGAGGUUGUGAGGCGCUGCCCCCACCAUGAGCGCUGCUCAGAUAGCGAUGGUCUGGCCCCUCCUCAGCAUCUUAUCCGAGUGG
AAGGAAAUUUGCGUGUGGAGUAUUUGGAUGACAGAAACACUUUUCGACAUAGUGUGGUGGUGCCCUAUGAGCCGCCUGAGGUU
GGCUCUGACUGUACCACCAUCCACUACAACUACAUGUGUAACAGUUCCUGCAUGGGCGGCAUGAACCGGAGGCCCAUCCUCAC
CAUCAUCACACUGGAAGACUCCAGUGGUAAUCUACUGGGACGGAACAGCUUUGAGGUGCGUGUUUGUGCCUGUCCUGGGAGAG
ACCGGCGCACAGAGGAAGAGAAUCUCCGCAAGAAAGGGGAGCCUCACCACGAGCUGCCCCCAGGGAGCACUAAGCGAGCACUG
CCCAACAACACCAGCUCCUCUCCCCAGCCAAAGAAGAAACCACUGGAUGGAGAAUAUUUCACCCUUCAGAUCCGUGGGCGUGA
GCGCUUCGAGAUGUUCCGAGAGCUGAAUGAGGCCUUGGAACUCAAGGAUGCCCAGGCUGGGAAGGAGCCAGGGGGGAGCAGGG
CUCACUCCAGCCACCUGAAGUCCAAAAAGGGUCAGUCUACCUCCCGCCAUAAAAAACUCAUGUUCAAGACAGAAGGGCCUGAC
UCAGACUGACAUUCUCCACUUCUUGUUCCCCACUGACAGCCUCCCACCCCCAUCUCUCCCUCCCCUGCCAUUUUGGGUUUUGG
GUCUUUGAACCCUUGCUUGCAAUAGGUGUGCGUCAGAAGCACCCAGGACUUCCAUUUGCUUUGUCCCGGGGCUCCACUGAACA
AGUUGGCCUGCACUGGUGUUUUGUUGUGGGGAGGAGGAUGGGGAGUAGGACAUACCAGCUUAGAUUUUAAGGUUUUUACUGUG
AGGGAUGUUUGGGAGAUGUAAGAAAUGUUCUUGCAGUUAAGGGUUAGUUUACAAUCAGCCACAUUCUAGGUAGGGGCCCACUU
CACCGUACUAACCAGGGAAGCUGUCCCUCACUGUUGAAUUUUCUCUAACUUCAAGGCCCAUAUCUGUGAAAUGCUGGCAUUUG
CACCUACCUCACAGAGUGCAUUGUGAGGGUUAAUGAAAUAAUGUACAUCUGGCCUUGAAACCACCUUUUAUUACAUGGGGUCU
AGAACUUGACCCCCUUGAGGGUGCUUGUUCCCUCUCCCUGUUGGUCGGUGGGUUGGUAGUUUCUACAGUUGGGCAGCUGGUUA
GGUAGAGGGAGUUGUCAAGUCUCUGCUGGCCCAGCCAAACCCUGUCUGACAACCUCUUGGUGAACCUUAGUACCUAAAAGGAA
AUCUCACCCCAUCCCACACCCUGGAGGAUUUCAUCUCUUGUAUAUGAUGAUCUGGAUCCACCAAGACUUGUUUUAUGCUCAGG
GUCAAUUUCUUUUUUCUUUUUUUUUUUUUUUUUUCUUUUUCUUUGAGACUGGGUCUCGCUUUGUUGCCCAGGCUGGAGUGGAG
UGGCGUGAUCUUGGCUUACUGCAGCCUUUGCCUCCCCGGCUCGAGCAGUCCUGCCUCAGCCUCCGGAGUAGCUGGGACCACAG
GUUCAUGCCACCAUGGCCAGCCAACUUUUGCAUGUUUUGUAGAGAUGGGGUCUCACAGUGUUGCCCAGGCUGGUCUCAAACUC
CUGGGCUCAGGCGAUCCACCUGUCUCAGCCUCCCAGAGUGCUGGGAUUACAAUUGUGAGCCACCACGUCCAGCUGGAAGGGUC
AACAUCUUUUACAUUCUGCAAGCACAUCUGCAUUUUCACCCCACCCUUCCCCUCCUUCUCCCUUUUUAUAUCCCAUUUUUAUA
UCGAUCUCUUAUUUUACAAUAAAACUUUGCUGCCA

### R337H TP53 CDS RNA Pathogenic Mutant

SEQ ID NO: 34 provides a nucleic acid sequence scenario with CDS encoding a variant of a
putatively pathogenic R337H mutant p53 protein of Homo sapiens
AUGGAGGAGCCGCAGUCAGAUCCUAGCGUCGAGCCCCCUCUGAGUCAGGAAACAUUUUCAGACCUAUGGAAACUACUUCCUGA
AAACAACGUUCUGUCCCCCUUGCCGUCCCAAGCAAUGGAUGAUUUGAUGCUGUCCCCGGACGAUAUUGAACAAUGGUUCACUG
AAGACCCAGGUCCAGAUGAAGCUCCCAGAAUGCCAGAGGCUGCUCCCCCCGUGGCCCCUGCACCAGCAGCUCCUACACCGGCG
GCCCCUGCACCAGCCCCCUCCUGGCCCCUGUCAUCUUCUGUCCCUUCCCAGAAAACCUACCAGGGCAGCUACGGUUUCCGUCU
GGGCUUCUUGCAUUCUGGGACAGCCAAGUCUGUGACUUGCACGUACUCCCCUGCCCUCAACAAGAUGUUUUGCCAACUGGCCA
AGACCUGCCCUGUGCAGCUGUGGGUUGAUUCCACACCCCCGCCCGGCACCCGCGUCCGCGCCAUGGCCAUCUACAAGCAGUCA
CAGCACAUGACGGAGGUUGUGAGGCGCUGCCCCCACCAUGAGCGCUGCUCAGAUAGCGAUGGUCUGGCCCCUCCUCAGCAUCU
UAUCCGAGUGGAAGGAAAUUUGCGUGUGGAGUAUUUGGAUGACAGAAACACUUUUCGACAUAGUGUGGUGGUGCCCUAUGAGC
CGCCUGAGGUUGGCUCUGACUGUACCACCAUCCACUACAACUACAUGUGUAACAGUUCCUGCAUGGGCGGCAUGAACCGGAGG
CCCAUCCUCACCAUCAUCACACUGGAAGACUCCAGUGGUAAUCUACUGGGACGGAACAGCUUUGAGGUGCGUGUUUGUGCCUG
UCCUGGGAGAGACCGGCGCACAGAGGAAGAGAAUCUCCGCAAGAAAGGGGAGCCUCACCACGAGCUGCCCCCAGGGAGCACUA
AGCGAGCACUGCCCAACAACACCAGCUCCUCUCCCCAGCCAAAGAAGAAACCACUGGAUGGAGAAUAUUUCACCCUUCAGAUC

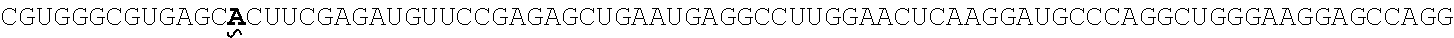

GGGGAGCAGGGCUCACUCCAGCCACCUGAAGUCCAAAAAGGGUCAGUCUACCUCCCGCCAUAAAAAACUCAUGUUCAAGACAG
AAGGGCCUGACUCAGACUGA

A Selected ASO for a simulated R337H TP53 pathogenic mutant with bold with wavy underline indicating variation site of interest:

SEQ ID NO: 142 provides a sequence for design of an antisense oligonucleotide sequence
corresponding to a doubly-minimal signature of a simulated R337H mutated p53 of Homo
sapiens

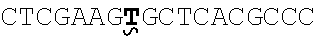

TP53 UTR5:
SEQ ID NO: 143 provides the polynucleotide sequence of a TP53 5' untranslated region of
Homo sapiens
AUUCGUGAGGGGUUUGUAAUGCAGGGCUGAGGAGUGUCCGAAGAGAAUGGGCAGGUGAGCGGUGAGACAGUUGUUCUUCCAGA
AGCUUUGCAGUGAAAGGAAUCAAAGAAAUGGAGCCGUGUAUCAGCAGCCAGACUGCCUUCCGGGUCACUGCC
TP53 UTR3:
SEQ ID NO: 144 provides the polynucleotide sequence of a TP53 3' untranslated region of
Homo sapiens
CAUUCUCCACUUCUUGUUCCCCACUGACAGCCUCCCACCCCCAUCUCUCCCUCCCCUGCCAUUUUGGGUUUUGGGUCUUUGAA
CCCUUGCUUGCAAUAGGUGUGCGUCAGAAGCACCCAGGACUUCCAUUUGCUUUGUCCCGGGGCUCCACUGAACAAGUUGGCCU
GCACUGGUGUUUUGUUGUGGGGAGGAGGAUGGGGAGUAGGACAUACCAGCUUAGAUUUUAAGGUUUUUACUGUGAGGGAUGUU
UGGGAGAUGUAAGAAAUGUUCUUGCAGUUAAGGGUUAGUUUACAAUCAGCCACAUUCUAGGUAGGGGCCCACUUCACCGUACU
AACCAGGGAAGCUGUCCCUCACUGUUGAAUUUUCUCUAACUUCAAGGCCCAUAUCUGUGAAAUGCUGGCAUUUGCACCUACCU
CACAGAGUGCAUUGUGAGGGUUAAUGAAAUAAUGUACAUCUGGCCUUGAAACCACCUUUUAUUACAUGGGGUCUAGAACUUGA
CCCCCUUGAGGGUGCUUGUUCCCUCUCCCUGUUGGUCGGUGGGUUGGUAGUUUCUACAGUUGGGCAGCUGGUUAGGUAGAGGG
AGUUGUCAAGUCUCUGCUGGCCCAGCCAAACCCUGUCUGACAACCUCUUGGUGAACCUUAGUACCUAAAAGGAAAUCUCACCC
CAUCCCACACCCUGGAGGAUUUCAUCUCUUGUAUAUGAUGAUCUGGAUCCACCAAGACUUGUUUUAUGCUCAGGGUCAAUUUC
UUUUUUCUUUUUUUUUUUUUUUUUUCUUUUUCUUUGAGACUGGGUCUCGCUUUGUUGCCCAGGCUGGAGUGGAGUGGCGUGAU
CUUGGCUUACUGCAGCCUUUGCCUCCCCGGCUCGAGCAGUCCUGCCUCAGCCUCCGGAGUAGCUGGGACCACAGGUUCAUGCC
ACCAUGGCCAGCCAACUUUUGCAUGUUUUGUAGAGAUGGGGUCUCACAGUGUUGCCCAGGCUGGUCUCAAACUCCUGGGCUCA
GGCGAUCCACCUGUCUCAGCCUCCCAGAGUGCUGGGAUUACAAUUGUGAGCCACCACGUCCAGCUGGAAGGGUCAACAUCUUU
UACAUUCUGCAAGCACAUCUGCAUUUUCACCCCACCCUUCCCCUCCUUCUCCCUUUUUAUAUCCCAUUUUUAUAUCGAUCUCU
UAUUUUACAAUAAAACUUUGCUGCCA

### TP53 UTR5:

SEQ ID NO: 143 provides the polynucleotide sequence of a TP53 5′ untranslated
region of Homo sapiens

AUUCGUGAGGGGUUUGUAAUGCAGGGCUGAGGAGUGUCCGAAGAGAAUGGGCAGGUGAGCGGUGAGACAGUUGUUCUUCCAGA
AGCUUUGCAGUGAAAGGAAUCAAAGAAAUGGAGCCGUGUAUCAGCAGCCAGACUGCCUUCCGGGUCACUGCC

TP53 UTR3:

SEQ ID NO: 144 provides the polynucleotide sequence of a TP53 3′ untranslated region
of Homo sapiens

CAUUCUCCACUUCUUGUUCCCCACUGACAGCCUCCCACCCCCAUCUCUCCCUCCCCUGCCAUUUUGGGUUUUGGGUCUUUGAA
CCCUUGCUUGCAAUAGGUGUGCGUCAGAAGCACCCAGGACUUCCAUUUGCUUUGUCCCGGGGCUCCACUGAACAAGUUGGCCU
GCACUGGUGUUUUGUUGUGGGGAGGAGGAUGGGGAGUAGGACAUACCAGCUUAGAUUUUAAGGUUUUUACUGUGAGGGAUGUU
UGGGAGAUGUAAGAAAUGUUCUUGCAGUUAAGGGUUAGUUUACAAUCAGCCACAUUCUAGGUAGGGGCCCACUUCACCGUACU
AACCAGGGAAGCUGUCCCUCACUGUUGAAUUUUCUCUAACUUCAAGGCCCAUAUCUGUGAAAUGCUGGCAUUUGCACCUACCU
CACAGAGUGCAUUGUGAGGGUUAAUGAAAUAAUGUACAUCUGGCCUUGAAACCACCUUUUAUUACAUGGGGUCUAGAACUUGA
CCCCCUUGAGGGUGCUUGUUCCCUCUCCCUGUUGGUCGGUGGGUUGGUAGUUUCUACAGUUGGGCAGCUGGUUAGGUAGAGGG
AGUUGUCAAGUCUCUGCUGGCCCAGCCAAACCCUGUCUGACAACCUCUUGGUGAACCUUAGUACCUAAAAGGAAAUCUCACCC
CAUCCCACACCCUGGAGGAUUUCAUCUCUUGUAUAUGAUGAUCUGGAUCCACCAAGACUUGUUUUAUGCUCAGGGUCAAUUUC
UUUUUUCUUUUUUUUUUUUUUUUUUCUUUUUCUUUGAGACUGGGUCUCGCUUUGUUGCCCAGGCUGGAGUGGAGUGGCGUGAU
CUUGGCUUACUGCAGCCUUUGCCUCCCCGGCUCGAGCAGUCCUGCCUCAGCCUCCGGAGUAGCUGGGACCACAGGUUCAUGCC
ACCAUGGCCAGCCAACUUUUGCAUGUUUUGUAGAGAUGGGGUCUCACAGUGUUGCCCAGGCUGGUCUCAAACUCCUGGGCUCA
GGCGAUCCACCUGUCUCAGCCUCCCAGAGUGCUGGGAUUACAAUUGUGAGCCACCACGUCCAGCUGGAAGGGUCAACAUCUUU
UACAUUCUGCAAGCACAUCUGCAUUUUCACCCCACCCUUCCCCUCCUUCUCCCUUUUUAUAUCCCAUUUUUAUAUCGAUCUCU
UAUUUUACAAUAAAACUUUGCUGCCA

A UTR-sequence-and-structure-preserving mRNA encoding p53 (HL=0.384 hours) with gene-variation homologous region underlined.

SEQ ID NO: 145 provides a design for a mRNA sequence trained under
UTR-sequence-and-structure from Homo sapiens reference TP53 mRNA
constraints to a higher expression design level

AUUCGUGAGGGGUUUGUAAUGCAGGGCUGAGGAGUGUCCGAAGAGAAUGGGCAGGUGAGCGGUGAGACAGUUGUUCUUCCAGA
AGCUUUGCAGUGAAAGGAAUCAAAGAAAUGGAGCCGUGUAUCAGCAGCCAGACUGCCUUCCGGGUCACUGCCAUGGAGGAGCC
GCAGUCAGAUCCUAGCGUCGAGCCCCCUCUGAGCCAGGAAACCUUCAGUGACCUCUGGAAGUUGCUUCCUGAGAACAACGUUC
UGUCCCCCUUGCCGUCCCAAGCAAUGGAUGAUUUGAUGCUGUCCCCGGACGACAUCGAACAAUGGUUCACUGAAGAUCCAGGU
CCAGAUGAGGCUCCCAGAAUGCCGGAGGCGGCUCCCCCGGUGGCGCCUGCGCCGGCAGCGCCGACACCGGCGGCGCCGGCGCC
GGCGCCAUCCUGGCCGCUCUCUUCGUCGGUCCCUUCCCAGAAGACCUACCAGGGUAGUUAUGGUUUUCGUCUGGGGUUCUUGC
AUUCUGGGACAGCCAAGUCUGUGACAUGCACGUAUUCGCCAGCGCUGAACAAAAUGUUCUGCCAGCUGGCGAAAACGUGCCCA
GUGCAAUUGUGGGUCGAUUCGACCCCACCUCCGGGGACUCGUGUGCGGGCCAUGGCCAUCUACAAGCAGUCCCAACACAUGAC
UGAGGUGGUGCGGCGGUGCCCCCAUCACGAGCGGUGCUCCGACUCGGAUGGGUUGGCACCGCCGCAGCACCUCAUUCGUGUGG
AGGGAAAUCUGCGUGUAGAAUAUUUGGAUGACCGGAAUACUUUCCGGCAUUCGGUGGUGGUGCCAUAUGAGCCGCCUGAGGUU
GGGUCGGAUUGUACCACAAUUCACUAUAAUUAUAUGUGUAACUCUAGCUGUAUGGGCGGCAUGAACCGCCGGCCGAUUCUCAC
CAUAAUCACACUGGAAGACUCUAGUGGUAAUCUAUUGGGAAGGAACAGCUUUGAGGUGCGUGUUUGUGCCUGCCCUGGGAGAG
ACAGGCGGACCGAGGAGGAGAAUCUCCGCAAGAAAGGGGAGCCUCACCACGAGCUGCCCCCAGGGAGCACUAAGCGAGCACUG
CCCAACAACACCAGCUCCUCUCCCCAGCCAAAGAAGAAACCACUGGAUGGAGAAUAUUUCACCCUUCAGAUCCGUGGGCGUGA
GCGCUUCGAGAUGUUCCGAGAGCUGAAUGAGGCCUUGGAACUCAAGGAUGCCCAGGCUGGGAAGGAGCCAGGGGGGAGCAGGG
CUCACUCCAGCCACCUGAAGUCCAAAAAGGGUCAGUCUACCUCCCGCCAUAAAAAACUCAUGUUCAAGACAGAAGGGCCUGAC
UCAGACUGACAUUCUCCACUUCUUGUUCCCCACUGACAGCCUCCCACCCCCAUCUCUCCCUCCCCUGCCAUUUUGGGUUUUGG
GUCUUUGAACCCUUGCUUGCAAUAGGUGUGCGUCAGAAGCACCCAGGACUUCCAUUUGCUUUGUCCCGGGGCUCCACUGAACA
AGUUGGCCUGCACUGGUGUUUUGUUGUGGGGAGGAGGAUGGGGAGUAGGACAUACCAGCUUAGAUUUUAAGGUUUUUACUGUG
AGGGAUGUUUGGGAGAUGUAAGAAAUGUUCUUGCAGUUAAGGGUUAGUUUACAAUCAGCCACAUUCUAGGUAGGGGCCCACUU
CACCGUACUAACCAGGGAAGCUGUCCCUCACUGUUGAAUUUUCUCUAACUUCAAGGCCCAUAUCUGUGAAAUGCUGGCAUUUG
CACCUACCUCACAGAGUGCAUUGUGAGGGUUAAUGAAAUAAUGUACAUCUGGCCUUGAAACCACCUUUUAUUACAUGGGGUCU
AGAACUUGACCCCCUUGAGGGUGCUUGUUCCCUCUCCCUGUUGGUCGGUGGGUUGGUAGUUUCUACAGUUGGGCAGCUGGUUA
GGUAGAGGGAGUUGUCAAGUCUCUGCUGGCCCAGCCAAACCCUGUCUGACAACCUCUUGGUGAACCUUAGUACCUAAAAGGAA
AUCUCACCCCAUCCCACACCCUGGAGGAUUUCAUCUCUUGUAUAUGAUGAUCUGGAUCCACCAAGACUUGUUUUAUGCUCAGG
GUCAAUUUCUUUUUUCUUUUUUUUUUUUUUUUUUCUUUUUCUUUGAGACUGGGUCUCGCUUUGUUGCCCAGGCUGGAGUGGAG
UGGCGUGAUCUUGGCUUACUGCAGCCUUUGCCUCCCCGGCUCGAGCAGUCCUGCCUCAGCCUCCGGAGUAGCUGGGACCACAG
GUUCAUGCCACCAUGGCCAGCCAACUUUUGCAUGUUUUGUAGAGAUGGGGUCUCACAGUGUUGCCCAGGCUGGUCUCAAACUC
CUGGGCUCAGGCGAUCCACCUGUCUCAGCCUCCCAGAGUGCUGGGAUUACAAUUGUGAGCCACCACGUCCAGCUGGAAGGGUC
AACAUCUUUUACAUUCUGCAAGCACAUCUGCAUUUUCACCCCACCCUUCCCCUCCUUCUCCCUUUUUAUAUCCCAUUUUUAUA
UCGAUCUCUUAUUUUACAAUAAAACUUUGCUGCCA

Below shows the putative before and after binding of the coding regions that would otherwise be targeted by the example R337H mutation-targeting ASO. The nucleotide positions that are underlined are putatively less able to be bound by the example ASO with bold with way underline indicating fortifications and the gene variation-targeting site of the ASO in bold. Note that after fortification, the ability of the example ASO to bind to the fortified encoding is putatively significantly reduced.

GGGCGUGAGCGCUUCGAG <-- region of TP53 supplemental mRNA before fortification
CCCGCACTCGTGAAGCTC <-- reoriented R337H mutation-targeting ASO

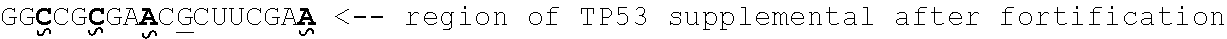

The below is a UTR-sequence-and-structure-preserving Supplemental mRNA (HL-0.383 hours) encoding p53 fortified (as shown above) against the example ASO with analogous targeted region underlined and fortifications shown in bold with wavy underline.

SEQ ID NO: 146 provides a design for a mRNA sequence trained under UTR-sequence-and-
structure constraints from Homo sapiens reference TP53 mRNA to a higher expression design
level and fortified against an R337H ASO design
AUUCGUGAGGGGUUUGUAAUGCAGGGCUGAGGAGUGUCCGAAGAGAAUGGGCAGGUGAGCGGUGAGACAGUUGUUCUUCCAGA
AGCUUUGCAGUGAAAGGAAUCAAAGAAAUGGAGCCGUGUAUCAGCAGCCAGACUGCCUUCCGGGUCACUGCCAUGGAGGAGCC
GCAGUCAGAUCCUAGCGUCGAGCCCCCUCUGAGCCAGGAAACCUUCAGUGACCUCUGGAAGUUGCUUCCUGAGAACAACGUUC
UGUCCCCCUUGCCGUCCCAAGCAAUGGAUGAUUUGAUGCUGUCCCCGGACGACAUCGAACAAUGGUUCACUGAAGAUCCAGGU
CCAGAUGAGGCUCCCAGAAUGCCGGAGGCGGCUCCCCCGGUGGCGCCUGCGCCGGCAGCGCCGACACCGGCGGCGCCGGCGCC
GGCGCCAUCCUGGCCGCUCUCUUCGUCGGUCCCUUCCCAGAAGACCUACCAGGGUAGUAUGGUUUUCGUCUGGGGUUCUUGC
AUUCUGGGACAGCCAAGUCUGUGACAUGCACGUAUUCGCCAGCGCUGAACAAAAUGUUCUGCCAGCUGGCGAAAACGUGCCCA
GUGCAAUUGUGGGUCGAUUCGACCCCACCUCCGGGGACUCGUGUGCGGGCCAUGGCCAUCUACAAGCAGUCCCAACACAUGAC
UGAGGUGGUGCGGCGGUGCCCCCAUCACGAGCGGUGCUCCGACUCGGAUGGGUUGGCACCGCCGCAGCACCUCAUUCGUGUGG
AGGGAAAUCUGCGUGUAGAAUAUUUGGAUGACCGGAAUACUUUCCGGCAUUCGGUGGUGGUGCCAUAUGAGCCGCCUGAGGUU
GGGUCGGAUUGUACCACAAUUCACUAUAAUUAUAUGUGUAACUCUAGCUGUAUGGGCGGCAUGAACCGCCGGCCGAUUCUCAC
CAUAAUCACACUGGAAGACUCUAGUGGUAAUCUAUUGGGAAGGAACAGCUUUGAGGUGCGUGUUUGUGCCUGCCCUGGGAGAG
ACAGGCGGACCGAGGAGGAGAAUCUCCGCAAGAAAGGGGAGCCUCACCACGAGCUGCCCCCAGGGAGCACUAAGCGAGCACUG

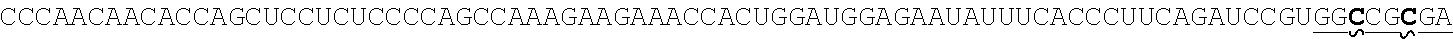

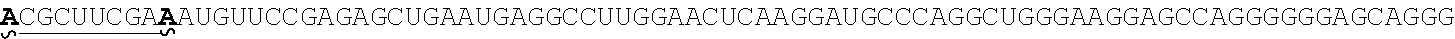

CUCACUCCAGCCACCUGAAGUCCAAAAAGGGUCAGUCUACCUCCCGCCAUAAAAAACUCAUGUUCAAGACAGAAGGGCCUGAC
UCAGACUGACAUUCUCCACUUCUUGUUCCCCACUGACAGCCUCCCACCCCCAUCUCUCCCUCCCCUGCCAUUUUGGGUUUUGG
GUCUUUGAACCCUUGCUUGCAAUAGGUGUGCGUCAGAAGCACCCAGGACUUCCAUUUGCUUUGUCCCGGGGCUCCACUGAACA
AGUUGGCCUGCACUGGUGUUUUGUUGUGGGGAGGAGGAUGGGGAGUAGGACAUACCAGCUUAGAUUUUAAGGUUUUUACUGUG
AGGGAUGUUUGGGAGAUGUAAGAAAUGUUCUUGCAGUUAAGGGUUAGUUUACAAUCAGCCACAUUCUAGGUAGGGGCCCACUU
CACCGUACUAACCAGGGAAGCUGUCCCUCACUGUUGAAUUUUCUCUAACUUCAAGGCCCAUAUCUGUGAAAUGCUGGCAUUUG
CACCUACCUCACAGAGUGCAUUGUGAGGGUUAAUGAAAUAAUGUACAUCUGGCCUUGAAACCACCUUUUAUUACAUGGGGUCU
AGAACUUGACCCCCUUGAGGGUGCUUGUUCCCUCUCCCUGUUGGUCGGUGGGUUGGUAGUUUCUACAGUUGGGCAGCUGGUUA
GGUAGAGGGAGUUGUCAAGUCUCUGCUGGCCCAGCCAAACCCUGUCUGACAACCUCUUGGUGAACCUUAGUACCUAAAAGGAA
AUCUCACCCCAUCCCACACCCUGGAGGAUUUCAUCUCUUGUAUAUGAUGAUCUGGAUCCACCAAGACUUGUUUUAUGCUCAGG
GUCAAUUUCUUUUUUCUUUUUUUUUUUUUUUUUUCUUUUUCUUUGAGACUGGGUCUCGCUUUGUUGCCCAGGCUGGAGUGGAG
UGGCGUGAUCUUGGCUUACUGCAGCCUUUGCCUCCCCGGCUCGAGCAGUCCUGCCUCAGCCUCCGGAGUAGCUGGGACCACAG
GUUCAUGCCACCAUGGCCAGCCAACUUUUGCAUGUUUUGUAGAGAUGGGGUCUCACAGUGUUGCCCAGGCUGGUCUCAAACUC
CUGGGCUCAGGCGAUCCACCUGUCUCAGCCUCCCAGAGUGCUGGGAUUACAAUUGUGAGCCACCACGUCCAGCUGGAAGGGUC
AACAUCUUUUACAUUCUGCAAGCACAUCUGCAUUUUCACCCCACCCUUCCCCUCCUUCUCCCUUUUUAUAUCCCAUUUUUAUA
UCGAUCUCUUAUUUUACAAUAAAACUUUGCUGCCA

### N. Example 14: Experimental Gene Training for Co-Expression Imbalance

Some genetic disorders are putatively caused by an imbalance of co-expression between proteins. For example, some sets of proteins form complexes. For example, PKD1 is said to form a complex with PKD2 proteins [Koyano et al.] and causing an imbalance between PKD1 and PKD2 causes autosomal dominant polycystic kidney disease (ADPKD) [Lakhia et al.]. The following is an example experimental approach to treat genetic disorders caused by co-expression imbalance, but not by decreased protein complex quantity.

Autosomal dominant polycystic kidney disease (ADPKD) is a highly prevalent disease.

Towards an experimental treatment design for ADPKD, based on a hypothesis that imbalance in the ratio of PKD proteins is the cause of ADPKD when a pathogenic mutation occurs in a PKD gene, the following is an example antisense gene training approach to restore the necessary co-expression of putatively necessary PKD proteins in individuals with heterozygous PKD1 state (one non-pathogenic variant together with one pathogenic mutant).

For the purposes of this co-expression example, let PKD1, PKD2 be the proteins that form a complex in an A:B co-expression ratio. Then, if there is a mutation of PKD1 in the heterozygous state that causes the available co-functional PKD1 quantity to drop by half (because of diploidy and the remaining non-pathological PKD1), then the ratio becomes A: 2B.

In some situations where there is natural synonymous variation in heterozygous state in PKD2, one may be able to restore the A:B co-expression ratio by specifically targeting one of the two putatively non-pathological variants of the PKD2 gene using MS-ASOs.

In the following, the non-targeted non-pathological PKD2 variant matches a sequence in the first of two non-pathological reference sets, while the targeted putatively non-pathological PKD2 variant matches a sequence in the second non-pathological reference set. A representation of a pair of PKD2 non-pathological variants using IUPAC codes with the site of variation in bold with wavy underline and an example site of variation of interest underlined.

SEQ ID NO: 147 provides a PKD2 mRNA scenario with a pair of nucleic acid sequences
representing synonymous putatively non-pathogenic variants via IUPAC variant codes encoding
a PKD2 protein from Homo sapiens

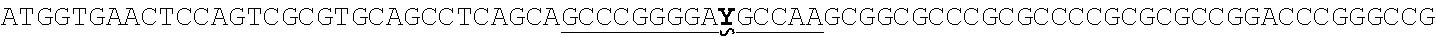

GCTGATGGCTGGCTGCGCGGCCGTGGGCGCCAGCCTCGCCGCCCCGGGCGGCCTCTGCGAGCAGCGGGGCCTGGAGATCGAGA
TGCAGCGCATCCGGCAGGCGGCCGCGCGGGACCCCCCGGCCGGAGCCGCGGCCTCCCCTTCTCCTCCGCTCTCGTCGTGCTCC
CGGCAGGCGTGGAGCCGCGATAACCCCGGCTTCGAGGCCGAGGAGGAGGAGGAGGAGGTGGAAGGGGAAGAAGGCGGAATGGT
GGTGGAGATGGACGTAGAGTGGCGCCCGGGCAGCCGGAGGTCGGCCGCCTCCTCGGCCGTGAGCTCCGTGGGCGCGCGGAGCC
GGGGGCTTGGGGGCTACCACGGCGCGGGCCACCCGAGCGGGAGGCGGCGCCGGCGAGAGGACCAGGGCCCGCCGTGCCCCAGC
CCAGTCGGCGGGGGGACCCGCTGCATCGCCACCTCCCCCTGGAAGGGCAGCCGCCCCGAGTGGCCTGGGCGGAGAGGCTGGT
TCGCGGGCTGCGAGGTCTCTGGGGAACAAGACTCATGGAGGAAAGCAGCACTAACCGAGAGAAATACCTTAAAAGTGTTTTAC
GGGAACTGGTCACATACCTCCTTTTTCTCATAGTCTTGTGCATCTTGACCTACGGCATGATGAGCTCCAATGTGTACTACTAC
ACCCGGATGATGTCACAGCTCTTCCTAGACACCCCCGTGTCCAAAACGGAGAAAACTAACTTTAAAACTCTGTCTTCCATGGA
AGACTTCTGGAAGTTCACAGAAGGCTCCTTATTGGATGGGCTGTACTGGAAGATGCAGCCCAGCAACCAGACTGAAGCTGACA
ACCGAAGTTTCATCTTCTATGAGAACCTGCTGTTAGGGGTTCCACGAATACGGCAACTCCGAGTCAGAAATGGATCCTGCTCT
ATCCCCCAGGACTTGAGAGATGAAATTAAAGAGTGCTATGATGTCTACTCTGTCAGTAGTGAAGATAGGGCTCCCTTTGGGCC
CCGAAATGGAACCGCTTGGATCTACACAAGTGAAAAAGACTTGAATGGTAGTAGCCACTGGGGAATCATTGCAACTTATAGTG
GAGCTGGCTATTATCTGGATTTGTCAAGAACAAGAGAGGAAACAGCTGCACAAGTTGCTAGCCTCAAGAAAAATGTCTGGCTG
GACCGAGGAACCAGGGCAACTTTTATTGACTTCTCAGTGTACAACGCCAACATTAACCTGTTCTGTGTGGTCAGGTTATTGGT
TGAATTCCCAGCAACAGGTGGTGTGATTCCATCTTGGCAATTTCAGCCTTTAAAGCTGATCCGATATGTCACAACTTTTGATT
TCTTCCTGGCAGCCTGTGAGATTATCTTTTGTTTCTTTATCTTTTACTATGTGGTGGAAGAGATATTGGAAATTCGCATTCAC
AAACTACACTATTTCAGGAGTTTCTGGAATTGTCTGGATGTTGTGATCGTTGTGCTGTCAGTGGTAGCTATAGGAATTAACAT
ATACAGAACATCAAATGTGGAGGTGCTACTACAGTTTCTGGAAGATCAAAATACTTTCCCCAACTTTGAGCATCTGGCATATT
GGCAGATACAGTTCAACAATATAGCTGCTGTCACAGTATTTTTTGTCTGGATTAAGCTCTTCAAATTCATCAATTTTAACAGG
ACCATGAGCCAGCTCTCGACAACCATGTCTCGATGTGCCAAAGACCTGTTTGGCTTTGCTATTATGTTCTTCATTATTTTCCT
AGCGTATGCTCAGTTGGCATACCTTGTCTTTGGCACTCAGGTCGATGACTTCAGTACTTTCCAAGAGTGTATCTTCACTCAAT
TCCGTATCATTTTGGGCGATATCAACTTTGCAGAGATTGAGGAAGCTAATCGAGTTTTGGGACCAATTTATTTCACTACATTT
GTGTTCTTTATGTTCTTCATTCTTTTGAATATGTTTTTGGCTATCATCAATGATACTTACTCTGAAGTGAAATCTGACTTGGC
ACAGCAGAAAGCTGAAATGGAACTCTCAGATCTTATCAGAAAGGGCTACCATAAAGCTTTGGTCAAACTAAAACTGAAAAAAA
ATACCGTGGATGACATTTCAGAGAGTCTGCGGCAAGGAGGAGGCAAGTTAAACTTTGACGAACTTCGACAAGATCTCAAAGGG
AAGGGCCATACTGATGCAGAGATTGAGGCAATATTCACAAAGTACGACCAAGATGGAGACCAAGAACTGACCGAACATGAACA
TCAGCAGATGAGAGACGACTTGGAGAAAGAGAGGGAGGACCTGGATTTGGATCACAGTTCTTTACCACGTCCCATGAGCAGCC
GAAGTTTCCCTCGAAGCCTGGATGACTCTGAGGAGGATGACGATGAAGATAGCGGACATAGCTCCAGAAGGAGGGGAAGCATT
TCTAGTGGCGTTTCTTACGAAGAGTTTCAAGTCCTGGTGAGACGAGTGGACCGGATGGAGCATTCCATCGGCAGCATAGTGTC
CAAGATTGACGCCGTGATCGTGAAGCTAGAGATTATGGAGCGAGCCAAACTGAAGAGGAGGGAGGTGCTGGGAAGGCTGTTGG
ATGGGGTGGCCGAGGATGAAAGGCTGGGTCGTGACAGTGAAATCCATAGGGAACAGATGGAACGGCTAGTACGTGAAGAGTTG
GAACGCTGGGAATCCGATGATGCAGCTTCCCAGATCAGTCATGGTTTAGGCACGCCAGTGGGACTAAATGGTCAACCTCGCCC
CAGAAGCTCCCGCCCATCTTCCTCCCAATCTACAGAAGGCATGGAAGGTGCAGGTGGAAATGGGAGTTCTAATGTCCACGTAT
GA

Because targeting the cytosine variant will not produce an MS-ASO with respect to the variation (recall targeting the C variant of a Y SNP, C or T, does not work) an MS-ASO targeting the thymine variant of PKD2 is picked (with the thymine-variant-targeting nucleotide of the MS-ASO in bold with wavy underline).

A thymine-variant-targeting PKD2 MS-ASO for experimental co-expression rebalancing treatment of ADPKD caused by a heterozygous PKD1 mutation:

SEQ ID NO: 148 provides a sequence for design of an antisense oligonucleotide corresponding
to a doubly-minimal signature of a simulated synonymous putatively non-pathogenic variant
encoding a PKD2 of Homo sapiens

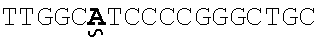

The purpose of targeting a single variant of PKD2 is to reduce the production of PKD2 by half to restore the ratio from A: 2B to A:B. One may also wish include an MS-ASO for the pathological variant of PKD1 if the pathological PKD1 variant is actively disruptive to the healthy formation of the PKD1/PKD2 complex.

### REFERENCES CITED

U.S. PATENTS

| Patent Number | Patent Date | Patentee |
|---|---|---|
| 11,739,317 | Aug. 29, 2023 | Barna et al. |
| U.S. Pat. No. 8,697,359 B1 | Apr. 15, 2014 | Zhang, Feng |
| U.S. Pat. No. 5,811,639A | Sep. 22, 1998 | Fullerton et al |
| U.S. Pat. No. 5,166,195 | Nov. 24, 1992 | Ecker, David J. |

U.S. APPLICATIONS

| Application Number | Application Date | Country |
|---|---|---|
| 17/184,505 | Feb. 24, 2021 | US |
| 17/364,890 | Jul. 1, 2021 | US |
| 18/273,293 | Jan. 20, 2022 | US |
| 18/546,698 | Jun. 30, 2021 | US |
| 18/546,698 | Jul. 1, 2021 | US |
| 11/869,973 | Oct. 10, 2007 | US |
| 18/586,232 | Feb. 23, 2024 | US |

### OTHER PUBLICATIONS

RMCE-Schlake et al. Use of mutated Flp-recognition-target-(FRT-)sites for the exchange of expression cassettes at defined chromosomal loci. Biochemistry. 33 (43), 1994, pp. 12746-12751.
Targeted RMCE-Bateman et al., Site-Specific Transformation of Drosophila via phiC31-Integrase-Mediated Cassette Exchange. Genetics. 173 (2), 2006, 769-777.
Multiplexing-Turan et al., Multiplexing RMCE: Versatile Extensions of Flp-Recombinase-Mediated Cassette-Exchange Technology. Journal of Molecular Biology. 402 (1), 2010, pp. 52-69.
RNAfold-Hofacker et al., Fast Folding and Comparison of Rna Secondary Structures. Monatsh Chem, 1994, 125, pp. 167-188.
CDSFold-Terai et al., CDSFold: An algorithm for designing a protein-coding sequence with the most stable secondary structure, Bioinformatics. vol. 32, No. 6, 2016, pp. 828-834. LinearDesign-Zhang et al., Algorithm for optimized mRNA design improves stability and immunogenicity, Nature, 621, 2023, pp. 396-403.
DegScore-https://github.com/eternagame/DegScore/commit/d6336915750f76b₂₉c2f4fb80a6006604c9bc468, dated Nov. 4, 2020.
DegScore-https://github.com/eternagame/DegScore/commit/0ddb3f3c8227266b₁d4c521757bb73915c4c96 Oa, dated Jun. 2, 2021.
Wayment-Steele et al. Theoretical basis for stabilizing messenger RNA through secondary structure design. bioRxiv. Aug. 24, 2020.
Wayment-Steele et al. Theoretical basis for stabilizing messenger RNA through secondary structure design. bioRxiv. Feb. 19, 2021.
Mauger et al. mRNA structure regulates protein expression through changes in functional half-life. Proceedings of the National Academy of Science, 116 (48), 24075-24083, Nov. 11, 2019.
Leppek et al., Combinatorial optimization of mRNA structure, stability, and translation for RNA-based therapeutics, Nature Communications, 13, Article number: 1536, 2022.
Jammali et al. SplicedFamAlign: CDS-to-gene spliced alignment and identification of transcript orthology groups, BMC Bioinformatics, 20, 2019
Kapustin et al. Splign: algorithms for computing spliced alignments with identification of paralogs. Biology Direct 3 (20), 2018.
Pooled Testing-Dorfman, Robert, The Detection of Defective Members of Large Populations, The Annals of Mathematical Statistics, 14 (4), 1943, 436-440.
Combinatorial Group Testing-Ding-Zhu et al., Combinatorial group testing and its applications. Singapore: World Scientific, 1993.
Probabilistic Group Testing-Sobel et al., Group testing to eliminate efficiently all defectives in a binomial sample, Bell System Technical Journal, 38 (5), 1959, pp. 1179-1252.
CRISPR-Carroll, A CRISPR Approach to Gene Targeting, Molecular Therapy. 20 (9), 2012, pp. 1658-1660.
RMCE-Osterwalder et al., Dual RMCE for efficient re-engineering of mouse mutant alleles, Nature Methods. 7 (11), 2010, pp. 893-895
Breeder's Equation-Kelly, J. K. The breeder's equation, Nature Education Knowledge 4 (5), 2011.
Population Genetics-Halliburton, Richard. Introduction to Population Genetics, 2004.
QTL Analysis-Paterson, Andrew H., Molecular Dissection of Complex Traits. 1998, CRC Press LLC
GWAS-Ioannidis, J.P., Validating, augmenting and refining genome-wide association signals, Nature Reviews Genetics. 10 (5), 2009, pp. 318-329
Engstrom et al., Promoter bashing, microRNAs, and Knox genes. New insights, regulators, and targets-of-regulation in the establishment of lateral organ polarity in Arabidopsis. Plant Physiology. 135 (2), 2004, pp. 685-694
Ag Pipeline-Cooper et al, Predicting the future of plant breeding: complementing empirical evaluation with genetic prediction. Crop & Pasture Science. 65, 2014, pp. 311-336.
Dwarf Corn-Schaefer, Christopher Michael, Breeding Potential of Semi-dwarf Corn for Grain and Forage in the Northern U.S. Corn Belt. 2010.
Dwarf Corn-Winkler et al. Physiological genetics of the dominant gibberellin-nonresponsive maize dwarfs, Dwarf8 and Dwarf9. Planta. 193, 1994, pp. 341-348.
Lee et al. The molecular basis of sulfonylurea herbicide resistance in tobacco, The EMBO Journal. 7 (5) pp. 1241-1248, 1998.
Green, Jerry, Review of Glyphosate and ALS-Inhibiting Herbicide Crop Resistance and Resistant Weed Management, Weed Technology. 21 (2), pp. 547-558, 2007.
Rakesh et al. Diversity of transgenes in sustainable management of insect pests, Transgenic Research. 32, pp. 351-381, 2023.
Duke et al. Genetic Engineering Crops for Improved Weed Management Traits, Crop Biotechnology. Chapter 6, pp. 52-66, 2002.
Rajasekaran et al. Genetic Engineering for Resistance to Phytopathogens, Crop Biotechnology. Chapter 9, pp. 97-117, 2002.
Ensembl Variants-https://ftp.ensemblgenomes.ebi.ac.uk/pub/plants/release-59/variation/vcf/zea_mays/zea_mays.vcf.gz.
NCBI Genomes-https://www.ncbi.nlm.nih.gov/datasets/genome/LCP LR-Fischer et al. Theoretical and Practical Improvements on the RMQ-Problem, with Applications to LCA and LCE. Combinatorial Pattern Matching. 2006, pp. 36-48
LCP-Kasai et al. Linear time longest-common-prefix computation in suffix arrays and its applications. In: Proceedings. CPM Volume 2089 of Lecture Notes in Computer Science., Springer 2001, pp. 181-192.
SA-Karkkainen et al. Simple Linear Work Suffix Array Construction. Proceedings of the 30th International Conference on Automata, Languages, and Programming, 2003, pp. 943-955. Presnyak et al. Codon Optimality is a Major Determinant of mRNA Stability. Cell. 160 (6), 2015, pp. 1111-1124.
Bokobza et al. miR-146b Protects the Perinatal Brain against Microglia-Induced Hypomyelination, Annals of Neurology. 91 (1), pp. 48-65, 2021.
Dillard et al. On the mechanism of tissue-specific mRNA delivery by selective organ targeting nanoparticles, Proceedings of the National Academy of Sciences. 118 (52) e2109256118, 2021. Kaksonen et al. Mechanisms of clathrin-mediated endocytosis, Nature Reviews Molecular Cell Biology, 19, pp. 313-326, 2018.
Gossen et al. Transcriptional activation by tetracyclines in mammalian cells, Science. 268 (5128), 1995, pp. 1766-1769.
Yao et al. Tetracycline repressor, tetR, rather than the tetR-mammalian cell transcription factor fusion derivatives, regulates inducible gene expression in mammalian cells, Human Gene Therapies, 9 (13), 1998, pp. 1939-1950.
Engel et al. Mechanisms and consequences of subcellular RNA localization across diverse cell types, Traffic. 21 (6), pp. 404-418, 2020.
Wang et al. RNA trafficking and subcellular localization—a review of mechanisms, experimental and predictive methodologies, Briefings in Bioinformatics. 24 (5), 2023. RFDiffusion-Watson et al. De novo design of protein structure and function with RFdiffusion, Nature. 620, pp. 1089-1100, 2023.
Benoit et al. Solanum pan-genetics reveals paralogues as contingencies in crop engineering, Nature. 640, pp. 135-145, 2025.
FRUITFULL-like genes regulate flowering time and inflorescence architecture in tomato, The Plant Cell. 34, pp. 1002-1019, 2022.
Bowman et al. Genes Directing Flower Development in Arabidopsis, The Plant Cell Online. 1 (1) pp. 37-52, 1989.
Riechmann et al. Dimerization Specificity of Arabidopsis MADS Domain Homeotic Proteins APETALA1, APETALA3, PISTILLATA, and AGAMOUS, Proceedings of the National Academy of Sciences of the United States of America. 93 (10), pp. 4793-4798, 1996.
Franken et al. The petunia MADS box gene FBP11 determines ovule identity, The Plant Cell. 7 (11), pp. 1859-1868, 1995.
von Rad et al. Two glucosyltransferases are involved in detoxification of benzoxazinoids in maize, The Plant Journal. 28 (6), pp. 633-642, 2001.
Honig et al. Chemical priming of plant defense responses to pathogen attacks, Frontiers in Plant Science. 14, 2023.
Waadt et al. Plant hormone regulation of abiotic stress responses, Nature Reviews Molecular Cell Biology. 23, pp. 680-694, 2022.
Sweetlove et al. The Plant Journal. 105 (2), pp. 283-557, 2021.
Yamada et al. RGF1 controls root meristem size through ROS signaling, Nature. 577 (7788), pp. 85-88, 2020.
Frankenthal et al. Cancer surveillance for patients with Fraumeni Syndrome in Brazil: A cost-effectiveness analysis, Lancet Regional Health Americas. 12, 2022
Tyner et al. p53 mutant mice that display early ageing-associated phenotypes. Nature. 415, 2002, pp. 45-53
Van Boxtel et al. Homozygous and Heterozygous p53 Knockout Rats Develop Metastasizing Sarcomas with High Frequency. The American Journal of Pathology. 179 (4), 2011, pp. 1616-1622.
Gencel-Augusto et al. p53 tetramerization: at the center of the dominant-negative effect of mutant p53. Genes & Development, 34 (17-18), 2020, 1128-1146.
Koyano et al. Pkd2, mutations linking to autosomal dominant polycystic kidney disease, localizes to the endoplasmic reticulum and regulates calcium signaling in fission yeast. Genes to Cells. 28 (11), 2023, pp. 811-820.
Lakhia et al. PKD1 and PKD2 mRNA cis-inhibition drives polycystic kidney disease progression. Nature Communications, 13, #4765, 2022.
Hadas et al. Optimizing Modified mRNA In Vitro Synthesis Protocol for Heart Gene Therapy. Molecular Therapy Methods Clinical Development, 14, 2019, pp. 300-305.
FDA GSRS-https://precision.fda.gov/uniisearch

1. A method for designing a sequence capable of aiming expression of a peptide to a desired relative expression level comprising:
obtaining two RNA sequences that encode the peptide, wherein one of the RNAs expresses at a greater level than the other;
selecting differences between the two RNA sequences, wherein a third variant encoding the peptide is obtained that differs from one of the two RNA sequences by the selected differences.

2. The method of claim 1, wherein the obtaining step is performed by:
obtaining one RNA sequence with an expression level greater than the desired relative expression level and obtaining another RNA sequence with an expression level less than the desired relative expression level.

3. The method of claim 1, wherein the selecting step is performed by:
identifying a difference as a change of codon encoding a corresponding amino acid within the sequence.

4. The method of claim 1, further comprising obtaining an RNA sequence from a reverse translation method.

5. The method of claim 4, wherein one or more reverse translation methods is selected from: CDSFold, and reference RNA.

6. The method of claim 1, further comprising designing a specification for treating an organism with an RNA molecule.

7. The method of claim 6, wherein the designing step comprises formulating the RNA molecule for medical use.

8. The method of claim 7, wherein the formulation step comprises combining the RNA molecule with one or more of: a buffer, a lubricant, a binder, a flavorant, a coating, and an adjuvant.

9. The method of claim 6, further comprising encapsulating the RNA molecule.

10. The method of claim 9, wherein a capsule is selected from: a virus, an adeno-associated virus, a viroid, a virion, a capsid, a micelle, a vesicle, a lipid nanoparticle, a protein nanoparticle, a DNA structure, and an RNA Structure.

11. The method of claim 10, wherein the capsule is decorated with transporter-specific proteins.

12. The method of claim 10, wherein the capsule is decorated with autologous HLA proteins.

13. The method of claim 1, the sequence design further comprising substituting at least one nucleotide analog for a native nucleotide in an RNA molecule.

14. The method of claim 13, wherein the at least one nucleotide analog is selected from: pseudouridine, inosine, I-methyl-pseudouridine, and 5-methyl-cytidine, 1-methoxy-pseudouridine, and pseudo-isocytidine.

15. The method of claim 6, wherein the treatment specification also includes an antisense oligonucleotide (ASO).

16. The method of claim 15, wherein the ASO is selective for a specific gene variant.

17. The method of claim 16, wherein the ASO is selective for one or more RNAs transcribed from the specific gene variant.

18. The method of claim 17, wherein a proper prefix of the ASO is not selective for an RNA transcribed from the specific gene variant.

19. The method of claim 17, wherein a proper suffix of the ASO is not selective for an RNA transcribed from the specific gene variant.

20. The method of claim 17, wherein neither a proper suffix nor a proper prefix of the ASO is selective for an RNA transcribed from the specific gene variant.

21. The method of claim 15, wherein the specification includes a plurality of ASOs.

22. The method of claim 6, wherein the design step comprises altering DNA in a genome to transcribe the RNA molecule for agricultural use.

23. The method of claim 22, wherein the specification of the altering comprises:
determining a difference between the desired RNA sequence and an existing RNA sequence encoding of the peptide;
mapping the difference to a DNA subsequence that is transcribed into the existing RNA sequence encoding the peptide; and
substituting nucleotides into a DNA molecule to create a substituted DNA molecule that transcribes the desired RNA sequence.

24. The method of claim 23, wherein the difference is determined by aligning the i^{th} nucleotide of the desired RNA sequence to the i^{th} nucleotide of the existing RNA sequence.

25. The method of claim 23, wherein the mapping is a set of DNA transcription intervals obtained from: transcription tracing, transformation tracking, or aligning the desired RNA sequence to the DNA sequence.

26. The method of claim 23, wherein the step of substituting nucleotides comprises one or more site-specific or site-directed methods from: DNA editing, recombinase mediated cassette exchange, and transformation.

27. The method of claim 26, wherein the DNA editing is selected from: genome editing, gene editing, prime editing, twin prime editing, and base editing.

28. The method of claim 22, further comprising designing sequences for training expression of a plurality of peptides.

29. The method of claim 28, wherein the plurality of peptides is selected using: a systems biology method, solver, simulation, or experimentation.

30. The method of claim 28, wherein an expression level of the plurality of peptides is recommended using: a systems biology method, solver, simulation, or experimentation.

31. The method of claim 28, wherein the expression level of the plurality of peptides is specified from a user interface.

32. The method of claim 28, wherein the designing of specifications for implementation of an altering step is carried out by a semi-automated, or partially-automated system, or fully-automated system.

33. The method of claim 32, wherein the semi-automated system, or partially-automated system, or fully-automated system carries out implementation in parallel.

34. The method of claim 33, wherein the implementation in parallel is selected from: multiplexing, and breeding.

35. The method of claim 34, wherein the breeding is selected from: trait integration, backcrossing, continuous breeding, speed breeding, forward breeding, and trait stacking.

36. A method for designing a sequence capable of aiming expression of a peptide to a desired relative expression level comprising:
obtaining two RNA sequences that encode the peptide, wherein one of the RNA sequences expresses at a greater level than the other;
selecting differences between the two RNA sequences, wherein a third RNA sequence variant encoding the peptide is obtained that differs from one of the two RNA sequences by the selected differences.

37. A one step computer-based method for computational construction of a first polypeptide-inducing polynucleotide sequence that induces a specified polypeptide wherein the one or more proteins comprising the specified polypeptide are capable of being expressed in vivo at a specified approximated desired relative expression level, wherein the method does not require an explicit starting sequence coding the specified polypeptide or does not require synthesis-based or experiment-based expression testing of polynucleotide sequences, comprising: specifying a desired relative expression level
and a set of constraints comprising the union of (a) a set of codon constraints that implicitly combinatorially describes a set of polypeptide-inducing polynucleotide sequences that induce the specified, and (b) a set of at least one nucleotide constraint independent of the codon constraints, whereby each nucleotide constraint forbids at least one of ‘A’, ‘C’, ‘G’, or ‘T’ at one or more specified positions in the resultant constructed sequence, whereby applying the one step computer-based method to computationally construct a polypeptide-inducing polynucleotide sequence satisfies the specified set of constraints whereby in vivo the resultant constructed polypeptide-inducing polynucleotide sequence is capable of being expressed at approximately the specified desired relative expression level.

38. The method of claim 37, further comprising the step of specifying producing in a cell the first polypeptide-inducing polynucleotide.

39. The method of claim 38, further comprising:
identifying a second polypeptide-inducing polynucleotide in a genome, wherein the second polypeptide-inducing polynucleotide induces the specified polypeptide; and
editing a genome of the cell using gene replacement or gene modification to comprise, transcribe, or transcribe then splice the first polypeptide-inducing polynucleotide in place of the second polypeptide-inducing polynucleotide.

40. The method of claim 38, wherein an mRNA encoding the specified polypeptide is produced by splicing of an RNA transcribed from the first polypeptide-inducing polynucleotide.

41. The method of claim 38, wherein an mRNA encoding the polypeptide is produced by transcription of the first polypeptide-inducing polynucleotide.

42. The method of claim 38, wherein an mRNA is the polypeptide-inducing polynucleotide.

43. The method of claim 37, wherein the desired relative expression level is or is correlated with a difference percentage used as the desired relative expression level.

44. The method of claim 37, wherein the set of at least one nucleotide constraint comprises a plurality of nucleotide constraints.

45. The method of claim 44, further comprising:
specifying a subsequence from one or more untranslated regions for the sequence using one or more constraints for generation of the sequence.

46. The method of claim 44, wherein the set of at least one nucleotide constraint comprises at least one nucleotide constraint that describes nucleotide polymorphisms not known to appear at the respective position of a homologous polynucleotide in germplasm of a species comprising a genome capable of leading to the translation of the specified polypeptide.

47. The method of claim 44, wherein the constraints are selected so that the generated first polypeptide-inducing polynucleotide and derivative RNA is fortified against an ASO.

48. A computer-aided method for designing a sequence for a second polypeptide-inducing polynucleotide that will help train expression of one or more proteins comprising an induced polypeptide to a desired relative expression level comprising:
obtaining a first sequence of a first polypeptide-inducing polynucleotide that induces the induced polypeptide via a first mRNA with first half-life estimate longer than or equal to the half-life estimate of a minimum MFE mRNA that encodes the induced polypeptide; and
generating the sequence for the second polypeptide-inducing polynucleotide that induces the induced polypeptide via a second mRNA with second half-life estimate, wherein the second mRNA design has half-life estimate longer than the first mRNA with first half-life estimate.

49. The method of claim 48, further comprising the step of specifying to introduce in cell the second polypeptide-inducing polynucleotide.

50. The method of claim 49, wherein the second mRNA with second half-life estimate is produced by splicing of an RNA transcribed from the second polypeptide-inducing polynucleotide.

51. The method of claim 49, wherein the second mRNA with second half-life estimate is produced by transcription of the second polypeptide-inducing polynucleotide.

52. The method of claim 49, further comprising:
identifying a third polypeptide-inducing polynucleotide sequence in a genome, wherein the third polypeptide-inducing polynucleotide induces the induced polypeptide; and
editing the genome of the cell using gene replacement or gene modification to comprise, transcribe, or transcribe then splice the second polypeptide-inducing polynucleotide in place of the third polypeptide-inducing polynucleotide.

53. The method of claim 52, wherein the third polypeptide-inducing polynucleotide is the same as the first polypeptide-inducing polynucleotide.

54. The method of claim 48, wherein a difference subset is selected so that the generated second polypeptide-inducing polynucleotide and derivative RNA is fortified against an ASO.

55. A method for designing a sequence for a first polypeptide-inducing polynucleotide that helps train expression of one or more proteins comprising a polypeptide to a desired relative expression level comprising:
obtaining two sequences representing RNAs encoding the polypeptide, wherein one of the sequences has a greater expression design level than the other; and
selecting differences between the sequences, wherein a polypeptide-inducing polynucleotide sequence that induces the polypeptide is generated from a third RNA sequence design, wherein the third RNA sequence differs from one of the first two RNA sequences by the selected differences, wherein an RNA of the third RNA sequence design has an expression design level intermediate of the expression design levels of the RNAs of the first two sequences.

56. The method of claim 55, wherein the method is computer-aided.

57. The method of claim 55, further comprising specifying to introduce in cell an mRNA comprising the RNA of the third sequence, wherein the mRNA comprises, is transcribed from, or is spliced from a transcript from the first polypeptide-inducing polynucleotide.

58. The method of claim 57, wherein the mRNA is producible via transcription.

59. The method of claim 57, wherein the mRNA is producible via a splice of a transcript.

60. The method of claim 55, wherein either or both sequences of the obtaining step represent an existing RNA, wherein the expression level of each existing RNA differs from the desired expression level.

61. The method of claim 60, wherein either or both existing RNA is considered wildtype.

62. The method of claim 55, wherein either or both of sequences of the obtaining step is considered a reference or consensus sequence.

63. The method of claim 55, wherein an RNA of the obtaining step has a minimum or minimal length-adjusted MFE, optionally subject to a set of constraints.

64. The method of claim 55, wherein an RNA of the obtaining step has a maximum or maximal length-adjusted MFE, optionally subject to a set of constraints.

65. The method of claim 55, wherein the RNAs of the obtaining step are mRNAs.

66. The method of claim 65, wherein a sequence of an mRNA of the obtaining step has a half-life estimate longer than an estimated half-life of an mRNA whose sequence has a minimum or minimal length-adjusted MFE, optionally subject to a set of constraints.

67. The method of claim 65, wherein an mRNA of the obtaining step has a half-life estimate shorter than an estimated half-life of an mRNA with a maximal length-adjusted MFE, optionally subject to a set of constraints.

68. The method of claim 55, wherein the selecting step comprises:
selecting a difference set, wherein an indice of a difference in the difference set may identify a codon position in its codon sequence, wherein the codon sequence represents the respective coding region.

69. The method of claim 55, wherein the selecting step comprises:
selecting a difference set wherein an indice of a difference in the difference set identifies the index of a nucleotide in the respective sequence.

70. The method of claim 65, wherein an amino acid sequence of a protein encoded by a first RNA is the same as an amino acid sequence of a protein encoded by a second RNA.

71. The method of claim 65, wherein one or more untranslated regions of the first RNA are the same as the respective untranslated region(s) of the second RNA.

72. The method of claim 57, further comprising:
identifying a second polypeptide-inducing polynucleotide in a genome that is transcribed or transcribed then spliced to an mRNA comprising one of the two RNA sequences of the obtaining step; and
editing the genome of the cell using gene replacement or gene modification to comprise the first polypeptide-inducing polynucleotide in place of the second polypeptide-inducing polynucleotide.

73. The method of claim 57, wherein a difference subset is selected so that a generated polypeptide-inducing polynucleotide and derivative RNA is fortified against an ASO.

74. A method for designing a sequence for a polypeptide-inducing polynucleotide that aims expression of one or more proteins comprising a polypeptide to a desired relative expression level comprising:
generating a series of sequences, wherein each sequence in the series is derived from a selected difference set, wherein every difference set that precedes another difference set in the series is a subset thereof, or wherein every difference set that succeeds another difference set in the series is a subset thereof.

75. A computer-based method for designing a sequence of a polypeptide-inducing polynucleotide that helps aim expression of one or more proteins comprising a polypeptide to a desired relative expression level comprising:
identifying a first sequence of a polynucleotide that induces the polypeptide;
generating a second sequence of a polynucleotide that induces the polypeptide;
computationally testing the second sequence to affirm invariance of a computed property with respect to the first sequence.

76. A method for designing a sequence of a polypeptide-inducing polynucleotide that aims expression of one or more proteins comprising a polypeptide to a desired relative expression level comprising:
applying the method of claim 75 more than once, wherein each test can be considered to be of a set of elements that differ between first sequence and second sequence, wherein a set of applications of the method comprises a method of group testing.

77. A method for designing a sequence for a polypeptide-inducing polynucleotide that aims expression of one or more proteins comprising a polypeptide to a desired relative expression level, wherein method does not use but may optionally be enhanced by synthesis-based or experiment-based expression testing, comprising: generating a series of polynucleotide sequences that induce the polypeptide, wherein each sequence of the series is derived from and described by a difference relative to a first sequence selected for the series, whereby the choices of each difference in the series comprise a search via a divide and conquer approach.

78. The method of claim 77, wherein the divide and conquer approach comprises a binary search.

79. The method of claim 77, wherein the divide and conquer approach comprises a level lowering approach.

80. A computer-aided method for designing a sequence of a second polypeptide-inducing polynucleotide that helps aim expression level of one or more proteins comprising a second polypeptide to an expression level of a first polypeptide, comprising:
identifying a sequence of a first polynucleotide that induces the first polypeptide via a first mRNA;
determining a half-life estimate of the first mRNA; and
generating a sequence for the second polypeptide-inducing polynucleotide that aims expression of the second polypeptide via a second mRNA that encodes the second polypeptide, wherein a half-life estimate of the second mRNA is approximately the same as the half-life estimate of the first mRNA.

81. The method of claim 80, further comprising specifying to produce in cell the second polypeptide-inducing polynucleotide.

82. The method of claim 81, further comprising:
identifying a third polynucleotide in a genome that is transcribed to, or is transcribed then spliced to the first mRNA; and
specifying edits to a genome of the cell via gene modification or gene replacement to comprise the second polypeptide-inducing polynucleotide in place of the third polynucleotide.

83. The method of claim 80, wherein the first mRNA is producible via transcription of the first polypeptide-inducing polynucleotide.

84. The method of claim 80, wherein the second polypeptide is functionally analogous to the first polypeptide.

85. The method of claim 84, wherein the second polypeptide and first polypeptide have the same enzymatic classification.

86. The method of claim 85, wherein the second polypeptide and first polypeptide have the same Enzyme Commission number.

87. The method of claim 80, wherein the second mRNA is mobility analogous to the first mRNA.

88. The method of claim 87, wherein the second mRNA and first mRNA have the same predicted subcellular localizations.

89. The method of claim 80, wherein the second mRNA is analogous to the first mRNA.

90. The method of claim 80, wherein the first polypeptide is a protein encoded by the first mRNA.

91. The method of claim 80, wherein the second polypeptide is a protein encoded by the second mRNA.

92. The method of claim 80, wherein sequences representing a pathogenicity-specific supplemental and derivative RNA is fortified against an ASO.

93. A method for designing a sequence of a polypeptide-inducing polynucleotide that aims expression of one or more proteins comprising a polypeptide to a specified relative expression level wherein the polypeptide-inducing polynucleotide is a CDS RNA and a quantity described by an expression design level is selected from or correlated with a negative length-adjusted MFE or a percentage.

94. A computer-based method for designing a sequence of a polypeptide-inducing polynucleotide that aims expression of one or more proteins comprising a polypeptide to a specified relative expression level without an explicit starting sequence coding the polypeptide or without in vitro or in vivo experimental iteration, wherein the polypeptide-inducing polynucleotide is an mRNA and a quantity described by an expression design level is selected from or correlated with a half-life, a negative length-adjusted MFE, or a percentage.

95. A computer-based method for redesigning a sequence of a polypeptide-inducing polynucleotide that aims expression of one or more proteins comprising a polypeptide to a specified relative expression design level without an explicit starting sequence coding the polypeptide or without in vitro or in vivo experimental iteration, wherein a genome altered by gene modification or gene replacement to comprise the polypeptide-inducing polynucleotide expresses the polypeptide at the specified relative expression design level without changing the associated promoter, wherein a quantity described by an expression design level is selected from or correlated with a protein production capacity per mRNA molecule, a half-life, a negative length-adjusted MFE, or a percentage.

96. A method for redesigning a sequence of a polypeptide-inducing polynucleotide that aims expression of one or more proteins comprising a polypeptide to a specified relative expression design level wherein a genome comprises the polypeptide-inducing polynucleotide and a quantity described by the expression design level is equal to or correlated with transcription initiation efficiency times protein production capacity per mRNA molecule.

97. A method for redesigning a sequence of a polypeptide-inducing polynucleotide that aims expression of one or more proteins comprising a polypeptide to a specified relative expression level wherein a resulting sequence retains one or more specified properties of an original sequence comprising:
identifying a first polypeptide-inducing polynucleotide sequence with the specified properties to use as an original sequence;
generating a second polypeptide-inducing polynucleotide sequence
testing a generated sequence for satisfaction of specified required properties; and
using the second polypeptide-inducing polynucleotide sequence in place of the first polypeptide-inducing polynucleotide sequence if the second polypeptide-inducing polynucleotide sequence satisfies the specified properties.

98. The method of claim 97, wherein a specified property is a subcellular locality or mobility property of the polynucleotide of the generated sequence.

99. The method of claim 97, wherein a specified property is a structural property of the polynucleotide of the generated sequence.

100. The method of claim 97, wherein a specified property is invariance of a computed secondary structure of untranslated regions with respect to the first polynucleotide sequence.

101. A method of designing a sequence for a pathogenicity-specific supplemental polynucleotide capable of being transcribed, optionally spliced, and translated in vivo to a therapeutic protein comprising a wildtype polypeptide from an intended donor whereby the pathogenicity-specific supplemental polynucleotide is to be used as part of a design of a DNA therapeutic for one or more intended recipients to help mute, reduce, delay, slow, treat, or prevent an autosomal recessive condition in an intended recipient, wherein the intended donor and the intended recipients are of the same species, comprising:
identifying an autosomal recessive condition in an intended recipient via two sequences of or from the same locus but from different chromosomal haploids in a genome of the intended recipient, wherein each of the two sequences carries a variant considered pathogenic for the condition when the variant is carried by both sequences;
selecting a subsequence of an intended donor genome, wherein the intended donor genome is different than the intended recipient genome, wherein the subsequence is homologous to the locus originating the autosomal recessive disorder in the intended recipient's genome, wherein the subsequence comprises homologous minimal promoter and exonic template regions of a chromosomal haploid of a locus of the intended donor genome, wherein the subsequence of the intended donor genome does not have a variant considered pathogenic, and confirming in cell that the DNA therapeutic produces one or more proteins comprising the wildtype polypeptide from the intended donor genome, wherein the one or more proteins is functionally analogous to a wildtype donor protein comprising the wildtype polypeptide from the intended donor.

102. The method of claim 101, wherein the subsequence of the intended donor genome is a contiguous sequence of the intended donor genome capable of producing a wildtype protein of the intended donor proteome, wherein the wildtype protein comprises the wildtype polypeptide of the intended donor proteome.

103. The method of claim 101, wherein the subsequence of the intended donor genome has an approximately same relative expression design level as a functionally analogous wildtype polynucleotide that encodes a wildtype donor protein comprising the wildtype polypeptide from the intended donor.

104. A method of designing a sequence for an antisense oligonucleotide that can be used to aim expression of one or more proteins comprising a polypeptide to some desired expression levels comprising:
identifying a subsequence of a target mRNA sequence that is a signature with respect to the target mRNA sequence and an intended treatment recipient's transcriptome and optionally genome; and
using a CSRC of the subsequence as a design of the antisense oligonucleotide sequence.

105. The method of claim 104, wherein the antisense oligonucleotide is a pareto-specific ASO.

106. The method of claim 105, wherein the antisense oligonucleotide is an MS-ASO.

107. The method of claim 104, wherein the antisense oligonucleotide sequence is pairing-sensitive to a transcript against a reference set comprising a transcriptome.

108. The method of claim 104, wherein the antisense oligonucleotide sequence is pairing-sensitive to a class of transcripts with a specific mutation against a reference set comprising a transcriptome.

109. The method of claim 104, wherein the method of identifying a signature comprises use of a suffix automaton, graph traversal, matrix multiplication, or an equivalent thereof.

110. A computer-aided method for a personalized on-target-off-target check of an ASO comprising:
obtaining sequences of a transcriptome of an intended recipient;
computing the loci or variants in the sequences of the transcriptome to which the ASO may bind; and
computing the loci or variants in a reference sequence using computational homology to determine whether the ASO is on target and whether the ASO may also target an unintended variant or locus.

111. A method for a personalized on-target-off-target check of an ASO comprising:
obtaining a sample of an intended treatment recipient;
performing one or more probe-based sequence or copy number assays to determine a number of loci or variants in sequences of a transcriptome to which the ASO may bind; and
computing the loci or variants in a reference sequence using computational homology to determine whether the ASO is on target and whether the ASO may also target an unintended variant or locus.

112. A method of configuring a specification of a delivery mechanism for a nucleic acid treatment comprising:
selecting a mechanism for delivering one or more nucleic acids;
wherein if the mechanism for delivering one or more nucleic acids is via encoding by a DNA structure or by a 3DNA, then the selection includes a promoter to condition the contexts of transcription of deliverable polynucleotides, wherein the promoter optionally allows for controlled transcription, wherein optionally the promoter becomes permissive of transcription initiation when supplied with a small molecule, wherein optionally the promoter is tetracycline-dependent and the small molecule is tetracycline; and
wherein if the mechanism for delivering one or more nucleic acids allows for polypeptide decorations, then the polypeptide decorations facilitate endocytosis of deliverable(s) and includes autologous human leukocyte antigen (HLA) proteins specific to an intended treatment recipient to help avoid immunogenicity, and optionally may also include one or more transporter-specific proteins specific to a target tissue or cell type.

113. The method of claim 112, wherein the mechanism for delivering one or more nucleic acids is selected from: a 3DNA, a DNA structure, a virus, an adeno-associated virus, a viroid, a virion, a capsid, a micelle, a vesicle, a lipid nanoparticle, a protein nanoparticle, and an RNA Structure.

114. The method of claim 113, wherein the mechanism for delivering one or more nucleic acids is a 3DNA.

115-120. (canceled)

121. A computer-aided method of screening an organism for suitability to treat the organism with a supplemental, suppressive, or multi-modal modality pathogenicity-specific DNA therapeutic that targets the root molecular cause and not just a symptom of a pathogenic genetic condition, without the use of gene editing proteins in the recipient, comprising:
detecting one or more sequences containing one or more pathogenic signatures and one or more locus signatures that uniquely identify the same locus of the one or more pathogenic signatures in the organism, wherein the pathogenic signatures indicates the root cause of the pathogenic genetic condition; and
affirming suitability of a modality if the modality is pathogenicity-specific and is among a list of best restorative modalities (BRMs) according to the inheritance phenotype of the pathogenic genetic condition with respect to the pathogenic signatures, wherein determination of a BRM is further refined by oligomeric participation, disruption, and/or haploinsufficiency information.

122. The method of claim 121, wherein if the list of acceptable modalities for a pathogenic genetic condition of the specific pathogenic sequences have the same Mendelian autosomal recessive inheritance phenotype, and the one or more pathogenic signatures comprise two or more pathogenic signatures from different chromosomal haploids of the uniquely identified locus, then a BRM of the affirming step comprises a pathogenicity-specific supplemental polypeptide-inducing polynucleotide.

123. The method of claim 121, wherein if the pathogenic genetic condition of the specific pathogenic sequences have the same X-linked recessive inheritance phenotype, and the one or more pathogenic signatures comprise a pathogenic signature from the active X chromosomal haploid of the uniquely identified locus, then a BRM of the affirming step comprises a pathogenicity-specific supplemental polypeptide-inducing polynucleotide.

124. The method of claim 121, wherein if the pathogenic genetic condition of a specific pathogenic variant has an autosomal dominant phenotype, then a BRM of the affirming step is composed of: (1) a pathogenicity-specific suppressive polynucleotide modality wherein each comprised suppressive is specific to an RNA of or originating a sequence containing a pathogenic signature, when a cause comprises active disruption by protein of or induced by the sequence containing a pathogenic signature; and/or (2) a suppressive polynucleotide modality wherein each comprised suppressive is specific to an RNA of a co-expressed polypeptide of no more than one chromosomal haploid per gene locus, wherein the co-expressed polypeptide is capable of forming a necessary oligomer with a product of RNA of the non-pathogenic allele of the locus of the gene with or inducing the sequence containing a pathogenic signature, such that balanced co-expression ratios are restored, when the cause comprises unbalanced co-expression; and (3) a pathogenicity-specific supplemental polypeptide-inducing polynucleotide modality that supplements via co-expression a set of polypeptide-inducing polynucleotides necessary to restore expression level sufficiency of properly formed and functionally necessary oligomers and maintaining balanced co-expression where necessary, when the cause comprises haploinsufficiency or comprising suppressive polynucleotides cause haploinsufficiency.

125. The method of claim 121, wherein if a pathogenic genetic condition of the specific pathogenic variant has X-linked dominant phenotype, then a BRM of the affirming step is composed of: (1) a pathogenicity-specific suppressive polynucleotide modality wherein each comprised suppressive is specific to an RNA of or originating a sequence containing a pathogenic signature, when the cause comprises active disruption by protein of or induced by the sequence containing the pathogenic signature; and/or (2) a suppressive polynucleotide modality wherein each comprised suppressive is specific to an RNA of a co-expressed polypeptide of no more than one chromosomal haploid per gene locus, wherein the co-expressed polypeptide is capable of forming a necessary oligomer with a product of RNA of the non-pathogenic allele of the locus of the gene with or inducing the sequence containing a pathogenic signature, such that balanced co-expression ratios are restored, when the cause comprises unbalanced co-expression; and (3) a pathogenicity-specific supplemental polypeptide-inducing polynucleotide modality that supplements via co-expression a set of polypeptide-inducing polynucleotides necessary to restore expression level sufficiency of properly formed and functionally necessary oligomers and maintaining balanced co-expression where necessary, when the cause comprises haploinsufficiency or comprising suppressive polynucleotides cause haploinsufficiency.

126. The method of claim 125, wherein if a BRM of the affirming step comprises a supplemental polypeptide-inducing polynucleotide, then the set of co-expressed supplemental polypeptide-inducing polynucleotides and derivative RNA are fortified against each included suppressive(s) as necessary.

127. The method of claim 121, wherein if the pathogenic genetic condition of the specific pathogenic variant has Y-linked, cytoplasmic inheritance, or incomplete-dominance, inheritance and is caused solely by a missing functional transcript then a BRM of the affirming step is a pathogenicity-specific supplemental polypeptide-inducing polynucleotide.

128. The method of claim 121, wherein if the pathogenic genetic condition of the specific pathogenic variant has Y-linked, cytoplasmic inheritance, or incomplete-dominance inheritance, and is caused solely by an actively disruptive transcript then a BRM of the affirming step is a pathogenicity-specific suppressive polynucleotide modality for RNA of or originating the pathogenic sequence.

129. The method of claim 121, wherein if the pathogenic genetic condition of the specific pathogenic variant has Y-linked, cytoplasmic inheritance, or incomplete-dominance, and is caused both by a missing functional transcript and by an actively disruptive transcript then a BRM of the affirming step is pathogenicity-specific multimodal, comprising a pathogenicity-specific suppressive polynucleotide for RNA of or originating the pathogenic sequence and a pathogenicity-specific supplemental polypeptide-inducing polynucleotide.

130. The method of claim 121, wherein if a pathogenicity-specific suppressive polynucleotide is required for a dominant genetic condition, but the targeted locus does not have distinct allelic sequences with respect to the intended recipient, then a pathogenicity-specific multimodal is a BRM of the affirming step only if it comprises co-delivery of a pathogenicity-specific suppressive polynucleotide and pathogenicity-specific supplemental polypeptide-inducing polynucleotide using a (sub) cellular co-delivery mechanism, to ensure atomicity of delivery of the supplemental and suppressive pathogenicity-specific polynucleotides to reduce or mitigate starting risks and discontinuation risks that may be associated with delivery only of the pathogenicity-specific suppressive.

131. The method of claim 124, wherein if a BRM of the affirming step is pathogenicity-specific multimodal, then the set of co-expressed pathogenicity-specific supplemental polynucleotides and derivative RNA are fortified against included suppressive(s) as necessary.

132-134. (canceled)

135. A method of designing a nucleic acid therapy for personalized medical treatment to reduce, mute, delay, slow, prevent, or treat an undesired genetic condition according to a pathogenicity-specific phenotype comprising:
one or more pathogenicity-specific supplemental polynucleotide or pathogenicity-specific suppressive polynucleotide for limiting the undesired genetic condition, wherein if the nucleic acid therapy includes both a suppressive polynucleotide and a supplemental polynucleotide then each included supplemental polynucleotide and derivative RNA is fortified against each deliverable suppressive polynucleotide.

136. The method of claim 135, wherein a pathogenicity-specific supplemental polynucleotide is included for limiting the undesired genetic condition.

137. The method of claim 135, wherein a pathogenicity-specific suppressive polynucleotide is part of the therapy for limiting the undesired genetic condition.

138. The method of claim 135, wherein a pathogenicity-specific supplemental polynucleotide and a suppressive polynucleotide is part of the therapy for limiting the undesired genetic condition.

139. The method of claim 135, wherein if a supplemental polynucleotide is included in the nucleic acid therapy and the supplemental polynucleotide encodes a wildtype protein, then a subcellular localization of the nucleic acid therapy is equal to or approximates the subcellular localization of a wildtype mRNA encoding a wildtype protein.

140. The method of claim 135, wherein the nucleic acid therapy further comprises a delivery mechanism.

141. The method of claim 140, wherein the delivery mechanism supports protein decorations.

142. The method of claim 141, wherein a protein decoration includes a protein to help direct deliverable(s) to specific tissue(s) and/or cell(s).

143. The method of claim 141, wherein a protein decoration includes one or more autologous human leukocyte antigen (HLA) proteins to help reduce immunogenicity.

144. The method of claim 140, wherein the delivery mechanism supports transcription of one or more comprising polynucleotides.

145. The method of claim 144, wherein transcription is dependent upon one or more promoters.

146. The method of claim 145, wherein one or more promoter is tetracycline-dependent.

147. The method of claim 144, wherein the delivery mechanism supports design of a stoichiometric delivery ratio for balancing co-expression.

148. The method of claim 140, wherein the delivery mechanism is selected from a 3DNA, a DNA structure, a virus, an adeno-associated virus, a viroid, a virion, a capsid, a micelle, a vesicle, a lipid nanoparticle, a protein nanoparticle, and an RNA Structure.

149. The method of claim 135, wherein the nucleic acid therapy is for an autosomal recessive disorder phenotype and the therapeutic deliverable consists of a pathogenicity-specific supplemental polynucleotide.

150. The method of claim 149, wherein the nucleic acid therapy comprises a supplemental polynucleotide of hemoglobin subunit beta (HBB) polypeptide-inducing polynucleotide for an autosomal recessive Sickle Cell genetic disorder.

151. The method of claim 149, wherein the nucleic acid therapy comprises a supplemental polynucleotide of hexosaminidase A (HEXA) polypeptide-inducing polynucleotide for an autosomal recessive Tay-Sachs genetic disorder.

152. The method of claim 135, wherein the nucleic acid therapy is for an X-linked recessive disorder phenotype and the therapeutic deliverable consists of a pathogenicity-specific supplemental polynucleotide.

153. The method of claim 135, wherein the nucleic acid therapy is for an autosomal dominant disorder phenotype and the treatment comprises a pathogenicity-specific suppressive polynucleotide.

154. The method of claim 153, wherein the nucleic acid therapy comprises a pathogenicity-specific suppressive polynucleotide for a pathogenic transthyretin (TTR) RNA for an autosomal dominant Familial Amyloid Polyneuropathy and/or Amyloidogenic Transthyretin Amyloidosis disorder.

155. The method of claim 153, wherein the nucleic acid therapy comprises a pathogenicity-specific suppressive polynucleotide for a pathogenic tumor protein p53 (TP53) RNA together with a supplemental polynucleotide of a non-pathogenic TP53 polypeptide-inducing polynucleotide for an autosomal dominant Li-Fraumeni syndrome disorder.

156. The method of claim 153, wherein the nucleic acid therapy comprising a co-expression balance restoring suppressive polynucleotide or multimodal involving PKD1 and/or PKD2 polypeptide-inducing polynucleotides for an autosomal dominant polycystic kidney disease (ADPKD) disorder.

157. The method of claim 135, wherein the nucleic acid therapy is for an X-linked dominant disorder phenotype and the deliverable comprises a pathogenicity-specific suppressive polynucleotide.

158. The method of claim 135, wherein the nucleic acid therapy is for a Y-linked disorder phenotype and the deliverable comprises a suppressive polynucleotide and a pathogenicity-specific supplemental polynucleotide fortified thereagainst.

159. The method of claim 135, wherein the nucleic acid therapy is for a disorder with cytoplasmic-inheritance phenotype.

160. The method of claim 135, wherein the nucleic acid therapy is for a disorder with incomplete dominance phenotype.

161. A computer-aided method of personalized medical treatment comprising:
screening a genome or transcriptome of an organism for one or more pathogenic sequences;
identifying one or more genetic conditions in the organism associated with one or more detected pathogenic sequences; and
obtaining one or more mending treatment sequence designs that are pathogenicity-specific for the detected pathogenic sequences; and
wherein if treatment includes an antisense oligonucleotide, then a personalized on-target-off-target check is performed for the antisense oligonucleotide with respect to an intended recipient using computational homology to a set of reference sequences to ensure no off-target site is accepted by the PSRC of the antisense oligonucleotide, to ensure on-target site(s) are accepted by the PSRC of the antisense oligonucleotide, and to ensure the maximum percent acceptance according to the PSRC of the antisense oligonucleotide over all off-target sites is the minimum possible for maximum safety, wherein a subset the set of reference sequences are obtained via sequence information from a transcriptome and/or genome of the intended recipient, wherein the sequence information comprises extensions of detected pathogenic sequences that are pairing sensitive to the antisense oligonucleotide, pairing sensitive to the maximum proper prefix of the antisense oligonucleotide, and pairing sensitive to the maximum proper suffix of the antisense oligonucleotide, wherein optionally the set of reference sequences may be obtained via sequencing and may include additional reference sequences of the species.

162. The method of claim 161, wherein the personalized on-target-off-target check is performed via probing of an intended recipient's transcriptome.

163. The method of claim 161, wherein the personalized on-target-off-target check is performed with sequences of a transcriptome of the intended recipient.

164. The method of claim 161, wherein the obtaining step further comprises communicating a signal to initiate retrieving, ordering, and/or producing a mending treatment sequence for the one or more detected pathogenic sequences of the screening step.

165. The method of claim 161, wherein the obtaining step further comprises:
retrieving information about or describing relevant pathogenic variant(s), relevant loci(us), relevant pathogenic genetic condition(s), relevant pathogenicity-specific polynucleotide treatment(s), and/or relevant, available, and/or producible mending treatments from a database, dictionary, index, catalogue, or other information store using a pathogenic signature and/or locus signature for clinical decision support to assist a healthcare provider with information about one or more genetic conditions of the intended recipient and to assist a healthcare provider in ascertaining specifically the efficacy, the safety, and the side effects that the specific intended recipient can expect from the one or more potential treatments for the specific genetic condition(s), wherein the efficacy, the safety, or the side effects vary with respect to a potential recipient.

166. The method of claim 161, further comprising a quality control step after the obtaining step wherein sequence designs and/or their metadata of the treatment are checked to ensure that an antisense oligonucleotide is not administered for a recessive pathogenic genetic condition, to ensure that a treatment intended for a dominant pathogenic genetic condition comprises an antisense oligonucleotide along with a PSRC of the antisense oligonucleotide and does not consist only of a supplemented polypeptide-inducing polynucleotide, ensure that the PSRC of the antisense oligonucleotide does not accept any off-target site in the intended recipient, and to ensure that if the PSRC of the antisense oligonucleotide accepts all the active alleles of a locus of the intended recipient then the treatment includes a co-delivered pathogenicity-specific supplemental polypeptide-inducing polynucleotide for the locus, wherein the pathogenicity-specific supplemental polypeptide-inducing polynucleotide is not accepted by the PSRC of the antisense oligonucleotide.

167. A method for identifying a likely splicing from one polynucleotide to a second polynucleotide, wherein the second polynucleotide is believed transcribed and spliced from the one polynucleotide, that utilizes dynamic programming together with longest common prefix queries between suffixes of sequences of the one polynucleotide and the second polynucleotide.

168. A method for designing a specification for altering DNA in a genome that helps aim expression of a polypeptide to a specified relative expression level comprising:
specifying a relative expression level as a quantity selected from or correlated with a protein production capacity per mRNA molecule, a half-life, a negative length-adjusted MFE, or a percentage; and
generating a polynucleotide sequence with the specified relative expression level for use in
gene modification or gene replacement of a gene within the genome.

169. The method of claim 168, further comprising:
identifying a transcription initiation efficiency of a promoter of a gene that induces the polypeptide; and
applying the method to aim expression.

170. The method of claim 168, wherein the altering step comprises:
determining a difference between a desired RNA sequence and an existing RNA sequence encoding of the polypeptide;
mapping the difference to a DNA subsequence that is transcribed into the existing RNA sequence encoding the polypeptide; and
identifying (poly)nucleotides to substitute into a DNA molecule to create a substituted DNA molecule that transcribes and/or splices the desired RNA sequence.

171. The method of claim 170, wherein the difference is determined by an order-preserving alignment that includes all the positions of the existing RNA sequence or an order-preserving alignment that includes all the positions of the desired RNA sequence.

172. The method of claim 170, wherein the mapping is via a set of DNA transcription intervals obtained from data from: tracing transcription, tracing splicing, tracking transformations, or aligning the existing RNA sequence to a DNA sequence.

173. The method of claim 172, wherein the set of DNA transcription intervals are determined from a likely splicing computed using dynamic programming together with longest common prefix queries between suffixes of the DNA subsequence and suffixes of the existing RNA sequence.

174. The method of claim 170, wherein the method of substitution of (poly)nucleotides identified comprises specification of one or more site-specific or site-directed methods from: DNA editing, recombinase mediated cassette exchange, and transformation.

175. The method of claim 174, wherein the specification of DNA editing method is selected from: genome editing, gene editing, prime editing, twin prime editing, and base editing.

176. The method of claim 174, wherein the specification of the DNA editing is selected from: gene modification and gene replacement.

177. The method of claim 168, further comprising computer-aided design of a plurality of polynucleotide sequences to help train expression of a plurality of proteins.

178. The method of claim 177, wherein the plurality of proteins is selected using: a systems biology method, solver, simulation, or experimentation.

179. The method of claim 177, wherein an expression design level of one or more of the plurality of polynucleotide sequences is a recommendation from: a systems biology method, solver, simulation, or experimentation.

180. The method of claim 178, wherein the expression design level of one or more of the polynucleotide sequences is specified from a user interface.

181. The method of claim 177, wherein the computer-aided design of the plurality of sequences with specified expression level design(s) is computer-automated.

182. The method of claim 181, wherein the computer-aided design of the plurality of sequences with specified expression level design(s) is carried out in parallel.

183. A computer-aided method for designing specifications for altering a genome to assess a novel polypeptide sequence, the method comprising:
identifying a sequence of a first potential polynucleotide capable of being transcribed (and optionally spliced) into a first mRNA encoding a first protein, wherein the first mRNA has a first half-life estimate;
generating a sequence of a second potential polynucleotide capable of being transcribed (and optionally spliced) into a second mRNA encoding the novel polypeptide sequence, wherein the second mRNA has approximately a same half-life as the first mRNA;
wherein a sequence of the first protein does not comprise the novel polypeptide; and
wherein alteration of a genome by gene modification or gene replacement to transcribe the second potential polynucleotide in place of the first potential polynucleotide enables assessment of the novel polypeptide sequence.

184. A computer-aided method for designing specifications for altering a genome to assess a novel expression level of a polypeptide, the method comprising:
identifying a sequence of a first polynucleotide capable of being transcribed (and optionally spliced) into a first mRNA encoding the polypeptide, wherein the first mRNA has a first calculated half-life estimate;
generating a sequence of a second polynucleotide capable of being transcribed (and optionally spliced) into a second mRNA encoding the polypeptide, wherein the second mRNA has a novel half-life, wherein the first calculated half-life estimate differs from the novel half-life;
wherein a sequence of the second mRNA differs from a sequence of the first mRNA according to an identity alignment; and
wherein alteration of a genome by gene modification or gene replacement to transcribe the second polynucleotide in place of the first polynucleotide enables assessment of the novel expression level.

185. A computer-based method for genomic prediction of phenotypes, wherein a genomic prediction model comprising
at least one computed relative expression design level estimate for at least one gene, polynucleotide, or polypeptide of interest
is used to infer at least one phenotype of interest.

186. A computer-based method for
performing a genotype to phenotype signal analysis wherein a genotype of at least one locus modeled in genotype to phenotype signal analysis is added or converted to model an independent variable comprising one or more computed relative expression design level estimate for at least one gene, polynucleotide, or polypeptide of interest.

187. (canceled)

188. A computer-aided method for determining a minimal set of polypeptide-inducing polynucleotides of a genome whose relative expression design level increases to all the polypeptide-inducing polynucleotides of the minimal set with respect to the genome enhances a biological pathway for the genome, the method comprising:
performing group testing on subsets of the set of polypeptide-inducing polynucleotides implicated by a signal analysis, wherein each test comprises an increase of relative expression design level of each of the polypeptide-inducing polynucleotides in the set of polypeptide-inducing polynucleotides implicated by the signal analysis, wherein the relative expression design level is implemented using gene modification or gene replacement.

189. A computer-aided method for designing specifications for altering a genome to produce a novel expression level of a polypeptide, the method comprising:
identifying a sequence of a first polynucleotide capable of being transcribed (and optionally spliced) into a first mRNA encoding the polypeptide, wherein the first mRNA has a first calculated half-life estimate;
generating a sequence of a second polynucleotide capable of being transcribed (and optionally spliced) into a second mRNA encoding the polypeptide, wherein the second mRNA has a novel half-life, wherein the first calculated half-life estimate differs from the novel half-life;
wherein a sequence of the second mRNA differs from a sequence of the first mRNA according to an identity alignment; and
wherein alteration of a genome by gene modification or gene replacement to transcribe the second polynucleotide in place of the first polynucleotide enables an altered genome to produce the polypeptide at the novel expression level in cell.

190. A computer-aided method for designing specifications for altering a genome to produce one or more proteins comprising a novel polypeptide variant at a desired expression design level, the method comprising:
identifying a sequence of a first polynucleotide capable of being transcribed (and optionally spliced) into a first mRNA encoding a first protein, wherein the first mRNA has a first calculated half-life estimate;
generating a sequence of a second polynucleotide capable of being transcribed (and optionally spliced) into a second mRNA encoding the novel polypeptide variant, wherein the second mRNA has a desired half-life;
wherein a sequence of the first protein does not comprise the novel polypeptide variant; and
wherein alteration of a genome by gene modification or gene replacement to transcribe the second polynucleotide in the place of the first polynucleotide enables an altered genome to produce a novel polypeptide variant at the desired expression design level in cell.

191. A method for designing specifications for introducing new genetic variation into a germplasm pool comprising claim 189 or claim 190.

192. A method of polynucleotide sequence design for improving the expression of one or more proteins comprising a target polypeptide to improve a performance capability of a genome of an organism for agriculture or carbon capture comprising:
identifying a sequence of a first polynucleotide that induces the target polypeptide and is capable of being transcribed to a first RNA wherein a first mRNA of the first RNA has first expression design level, wherein the target polypeptide is encoded by the first mRNA;
generating a sequence of a second polynucleotide that induces the target polypeptide and is capable of being transcribed to a second RNA wherein a second mRNA of the second RNA has second expression design level, wherein the target polypeptide is encoded by the second mRNA, wherein a protein produced from the second mRNA has an amino acid sequence that is identical to an amino acid sequence of a protein produced from the first mRNA, wherein the second expression design level differs from the first expression design level, wherein the second polynucleotide differs from the first polynucleotide according to an identity alignment;
wherein a genome changed to comprise the second polynucleotide expresses proteins comprising the target polypeptide in a greater quantity than in the scenario the genome comprised the first polynucleotide when a sequence of the second mRNA has greater expression design level than that of a sequence of the first mRNA, and wherein the second polynucleotide expresses the target polypeptide in lesser quantity than the first polynucleotide when the second mRNA sequence has lesser expression design level than that of a sequence of the first mRNA, whereby a difference in polypeptide expression improves a performance capability of an organism with a modified genome for agriculture or carbon capture; and
wherein the polynucleotide sequence design is performed by a method that comprises a method of claim 36, claim 37, claim 48, claim 55 or claim 167.

193. The method of claim 192, wherein if the second expression design level is greater than the first expression design level, then the change improves the performance capability of an organism with a changed genome.

194. The method of claim 192, wherein if the second expression design level is less than the first expression design level, then the change improves the performance capability of an organism with a changed genome.

195. The method of claim 192, wherein the second expression design level is aimed to a specified quantity.

196. The method of claim 195, wherein the specified quantity is determined from a sequence of an identified third polynucleotide that induces the target polypeptide and is capable of being transcribed to a third RNA wherein a third mRNA of the third RNA has third expression design level.

197. The method of claim 196, wherein the second expression design level is equal to the third expression design level.

198. The method of claim 196, wherein the second expression design level is greater than the third expression design level.

199. The method of claim 196, wherein the second expression design level is less than the third expression design level.

200. The method of claim 192, wherein a quantity described by each expression design level is or is correlated with a negative length-adjusted minimum free energy (MFE).

201. The method of claim 192, wherein a quantity described by each expression design level is or is correlated with a half-life.

202. The method of claim 192, wherein a quantity described by each expression design level is a percentage.

203. The method of claim 192, wherein a quantity described by each expression design level is or is correlated with a protein production capacity per mRNA molecule.

204. A method of describing an expression design level as a maximal transcription initiation efficiency when a promoter conditions transcription initiation on a presence of a small molecule.

205. The method of claim 192, wherein a quantity described by each expression design level is equal to transcription initiation efficiency times protein production capacity per mRNA molecule.

206. The method of claim 192, wherein if the change comprises a gene modification of the first polynucleotide to the second polynucleotide or a gene replacement of the first polynucleotide by the second polynucleotide, then the change improves the performance capability of an organism with a changed genome.

207. The method of claim 192, wherein the change comprises a gene insertion by way of gene modification or gene replacement.

208. The method of claim 192, wherein a polypeptide produced from the second mRNA has an amino acid sequence that is identical to an amino acid sequence of the polypeptide produced from the first mRNA.

209. The method of claim 192, wherein the first mRNA is wildtype and the second mRNA differs from wildtype.

210. The method of claim 209, wherein the target polypeptide is an enzyme of a hormone producing pathway, the performance capability of an organism with a changed genome is increased development, and the organism is of a type harvested for agriculture.

211. The method of claim 210, wherein the second mRNA has calculated half-life estimate lower than a calculated half-life estimate of the first mRNA, the enzyme comprises a maize GA20ox3, a stalk circumference has increased development, and the organism is a maize plant.

212. The method of claim 210, wherein the second mRNA sequence has higher expression design level than the sequence of the first mRNA, the enzyme comprises a maize GA20ox3, one or more florescence features have increased development in the absence of a damaging wind event, and the organism is a maize plant.

213. The method of claim 212, wherein the one or more florescence features comprise increased kernel row number.

214. The method of claim 212, wherein the florescence features comprise increased yield.

215. The method of claim 209, wherein the target polypeptide is an insect toxin, the performance capability of an organism with a changed genome is resistance to insect pests, and the organism is of a type harvested for agriculture.

216. The method of claim 215, wherein the second mRNA sequence has calculated half-life estimate longer than the calculated half-life estimate of the first mRNA, the insect toxin comprises a maize Cry3Bb1, the resistance is to Coleopteran, and the organism is a maize plant.

217. The method of claim 209, wherein the target polypeptide is an enzyme of a chemical defense pathway, the performance capability is resistance to bacteria, fungus, and/or pests, and the organism is of the type harvested for agriculture.

218. The method of claim 217 wherein the chemical defense pathway produces DIMBOA glucosides and the organism is a maize plant.

219. The method of claim 218, wherein the second mRNA sequence has higher expression design level than the sequence of the first mRNA and the target polypeptide comprises a maize DIMBOA UDP-glucosyltransferase.

220. The method of claim 192, wherein the target polypeptide is an enzyme designed for herbicide tolerance, the performance capability of an organism with a changed genome is tolerance to an herbicide.

221. The method of claim 220, wherein the second mRNA sequence has higher expression design level than the sequence of the first mRNA, the target polypeptide comprises a maize acetolactate synthase that was redesigned for herbicide tolerance, the herbicide comprises a sulfonylurea and/or triazolopyrimidine herbicide, and the organism is a maize plant.

222. The method of claim 209, wherein the second mRNA sequence has lower calculated half-life estimate than the first mRNA sequence, the target polypeptide is a fruit size regulator, and the performance capability of an organism with a changed genome is increased fruit size.

223. The method of claim 222, wherein the fruit size regulator is a CLAVATA3 and the organism is a tomato plant.

224. The method of claim 222, wherein the fruit size regulator is a CLAVATA3 and the organism is an eggplant plant.

225. The method of claim 209, wherein the second mRNA sequence has greater expression design level than the sequence of the first mRNA, the target polypeptide is a root growth factor inducible transcription factor, the performance capability of an organism with a changed genome is increased root growth, and the organism is a crop valued in part for root growth.

226. The method of claim 225, wherein the value crop valued in part is for carbon capture.

227. A method for designing a sequence for a polynucleotide to help aim expression of one or more proteins comprising a polypeptide in a cell, the method comprising:
obtaining a sequence of a potential first polynucleotide, wherein the first polynucleotide is capable of being transcribed to produce a first mRNA encoding a protein comprising the polypeptide, wherein the first mRNA has a first calculated half-life estimate;
generating a sequence of a potential second polynucleotide, wherein the second polynucleotide is capable of being transcribed into a second mRNA encoding the polypeptide, wherein the second mRNA has a second calculated half-life estimate that differs from the first calculated half-life estimate;
wherein the second mRNA differs from the first mRNA according to an order-preserving alignment with minimum difference; and
wherein if the second calculated half-life estimate is longer than the first calculated half-life estimate, then on average an encoded polypeptide will be produced in greater quantity by the second mRNA molecule in cell than would be produced by the first mRNA molecule in cell or if the second calculated half-life estimate is shorter than the first calculated half-life estimate, then on average an encoded polypeptide will be produced in lesser quantity by the second mRNA molecule in cell than would be produced by the first mRNA molecule in cell.

228. The method of claim 227, wherein the second calculated half-life estimate is longer than the first calculated half-life estimate.

229. The method of claim 227, wherein the second calculated half-life estimate is shorter than the first calculated half-life estimate.

230. The method of claim 227, wherein a protein produced from the second mRNA has an amino acid sequence that is the same as a protein produced from the first mRNA.

231. The method of claim 227, wherein the sequence of the first polynucleotide is a wildtype sequence.

232. A method for designing a sequence for a polynucleotide to help aim expression of one or more proteins comprising a polypeptide in a cell, the method comprising:
obtaining a sequence of a first polynucleotide capable of being transcribed to produce a first mRNA encoding a protein comprising the polypeptide, wherein the first mRNA has a first calculated half-life estimate;
obtaining a sequence of a second polynucleotide capable of being transcribed to produce a second mRNA encoding a protein comprising the polypeptide, wherein the second mRNA has a second calculated half-life estimate, and wherein a sequence of the second mRNA differs from a sequence of the first mRNA according to an order-preserving alignment with minimum size difference;
generating a sequence of a third polynucleotide capable of being transcribed to produce a third mRNA encoding a protein comprising the polypeptide, wherein the third mRNA has a third calculated half-life estimate, wherein a sequence of the third mRNA differs from the sequences of the first mRNA and second mRNA according to an identity alignment, wherein the third calculated half-life estimate is intermediate to the first calculated half-life estimate and the calculated second half-life estimate; and
wherein if the third calculated half-life estimate is longer than the first calculated half-life estimate, a set of proteins comprising the polypeptide resulting from transcription of the third polynucleotide will in aggregate be produced in greater quantity in cell than would be from the first polynucleotide in cell; or if the third calculated half-life estimate is shorter than the first calculated half-life estimate, then a set of proteins comprising the polypeptide resulting from transcription of the third polynucleotide will be produced in lesser quantity in cell than would be from the first polynucleotide in cell.

233. The method of claim 232, wherein the second mRNA has a maximal or minimum MFE for mRNAs encoding the polypeptide.

234. The method of claim 232, wherein the first mRNA has a maximal or minimum MFE for mRNAs encoding the polypeptide.

235. The method of claim 232, wherein a calculated MFE of the third mRNA is greater than a calculated MFE of the second mRNA.

236. The method of claim 232, wherein a calculated MFE of the third mRNA is less than a calculated MFE of the second mRNA.

237. The method of claim 232, wherein a protein produced from the third mRNA has an amino acid sequence that is the same as a protein produced from the first mRNA.

238. The method of claim 232, wherein one or both of the first and second polynucleotide sequences is a wildtype sequence.

239. A method for aiming expression of one or more proteins comprising a target polypeptide in a cell, the method comprising:
identifying a first sequence of a polynucleotide capable of being transcribed to produce a first mRNA encoding a protein comprising the target polypeptide, wherein the first mRNA has a first calculated half-life estimate;
generating a second sequence of a polynucleotide capable of being transcribed produce a second mRNA encoding a protein comprising the target polypeptide, wherein the second mRNA has a second calculated half-life estimate;
wherein the second mRNA varies from the first mRNA by one or more nucleotides and wherein, as a result of the nucleotide variations, the second calculated half-life estimate differs from the first calculated half-life estimate; and
wherein if residing in a cell the second polynucleotide the cell produces the target polypeptide, wherein if the second calculated half-life estimate is longer than the first calculated half-life estimate, then one or more proteins comprising the target polypeptide will be produced in greater quantity than if residing in a cell the first polynucleotide, or if the second calculated half-life estimate is shorter than the first calculated half-life estimate, then one or more proteins comprising the target polypeptide will be produced in lesser quantity than if producing in a cell the first polynucleotide.

240. The method of claim 239, wherein the method further comprises the step of delivering for entry into the cell a carrier of or comprising the second polynucleotide for health applications, or editing the cell genome to comprise the second polynucleotide in the place of an instance of the first polynucleotide whereby the edited genome transcribes from the second polynucleotide instead of from the first polynucleotide for agriculture applications.

241. The method of claim 239, wherein the first and second mRNAs each have a minimum free energy (MFE) estimate, and wherein the second mRNA has a MFE estimate that differs from the MFE estimate of the first mRNA as a result of the nucleotide sequence variations.

242. The method of claim 239, wherein the calculated MFE estimate of the sequence of the second mRNA is greater than the calculated MFE estimate of the sequence of the first mRNA.

243. The method of claim 239, wherein the calculated MFE estimate of the sequence of the second mRNA is less than the calculated MFE estimate of the sequence of the first mRNA.

244. The method of claim 239, wherein the second calculated half-life estimate is longer than the first calculated half-life estimate.

245. The method of claim 239, wherein the second calculated half-life estimate is shorter than the first calculated half-life estimate.

246. The method of claim 239, wherein a protein comprising the target polypeptide produced from the second polynucleotide has an amino acid sequence that is identical to a protein comprising the target polypeptide produced from the first polynucleotide.

247. The method of claim 239, wherein one or both of the first and second polynucleotidesequences is a wildtype sequence.

248. A method for aiming expression of one or more proteins comprising a target polypeptide in a cell, the method comprising:
identifying a sequence of a first polynucleotide capable of being transcribed to produce a first mRNA encoding a protein comprising the target polypeptide, wherein the first mRNA has a first minimum free energy (MFE) estimate;
identifying a sequence of a second polynucleotide capable of being transcribed to produce a second mRNA encoding a protein comprising the target polypeptide, wherein the second mRNA has a second MFE estimate, wherein the second mRNA sequence varies from the first mRNA sequence by one or more nucleotides, wherein the second MFE estimate differs from the first MFE estimate;
generating a sequence of a third polynucleotide capable of being transcribed to produce a third mRNA encoding a protein comprising the target polypeptide, wherein the third mRNA has a third MFE estimate, wherein the third mRNA sequence varies from the first and second mRNA sequences by one or more nucleotides, wherein the third MFE estimate is intermediate to the first MFE estimate and the second MFE estimate, wherein the generation does not require but may optionally be enhanced by non-coding region design;
wherein all else equal, if residing in a cell the third polynucleotide, whereby the cell is capable of producing one or more proteins comprising the target polypeptide, wherein if the third MFE estimate is less than the first MFE estimate, then one or more proteins comprising the target polypeptide will be produced in greater quantity by the third polynucleotide than would be from the first polynucleotide or if the third MFE estimate is greater than the first MFE estimate, then one or more proteins comprising the target polypeptide will be produced in lesser quantity by the third polynucleotide than would be from the first polynucleotide.

249. The method of claim 248, wherein the method further comprises the step of delivering for entry into the cell a carrier of or comprising the third nucleotide sequence for health applications, or editing the cell genome to comprise the third polynucleotide in place of an instance of the first or second polynucleotide whereby the edited genome transcribes from the second polynucleotide instead of from the first polynucleotide for agriculture applications.

250. The method of claim 248, wherein the first or second MFE estimate is a maximal or minimum MFE for mRNAs encoding the target polypeptide.

251. The method of claim 248, wherein the calculated MFE of the third mRNA sequence is greater than the calculated MFE of the first mRNA sequence.

252. The method of claim 248, wherein the calculated MFE of the third mRNA sequence is less than the calculated MFE of the first mRNA sequence.

253. The method of claim 248, wherein a protein comprising the target polypeptide produced from the third mRNA has an amino acid sequence that is identical to a protein comprising the target polypeptide produced from the first mRNA.

254. The method of claim 248, wherein one or both of the first polynucleotide and second polynucleotide sequences is a wildtype sequence.

255. A gene therapy design method, whereby the gene therapy helps prevent, mitigate, delay, slow, reduce, mute, lessen, or treat a target autosomal recessive genetic condition or disorder, comprising:
identifying a gene in which pathogenic variation is believed to modulate (be the root cause of) the target autosomal recessive genetic condition or disorder;
identifying the pathogenicity-specific supplemental polypeptide-inducing polynucleotide sequence comprising a contiguous sequence consisting of the promoter region (optionally minimal) and transcribable portion of a non-pathogenic functional version of the gene or generating a polynucleotide analogous thereto;
specifying or defaulting to a DNA structure, such as single-stranded or double-stranded linear DNA, plasmid DNA, DNA origami, 3DNA, or an analogue thereof, to comprise the polypeptide-inducing polynucleotide, wherein the transcribable structure is decorated with protein comprising a nuclear localization signal to facilitate subcellular localization for transcription, wherein optionally the promoter region of the polynucleotide is preceded in the DNA structure by a small molecule responsive element, such as TRE, for conditional transcription such as by tetracycline or doxycycline;
specifying or defaulting to a carrier to comprise the transcribable structure, or contain and release the transcribable structure, wherein the carrier is a lipid nanoparticle, a protein nanoparticle, DNA origami, another polynucleotide structure, or an analogue thereof, wherein the carrier is decorated by recipient-self-identifying protein, such as HLA protein autologous to the intended recipient, wherein the carrier is decorated by endocytosis-facilitating protein, such as clathrin protein;
wherein transcription of the transcribable portion of the polynucleotide in cell leads to production of mRNA with non-pathogenic wildtype subcellular localization capable of leading to translation of a therapeutic protein;

256. A gene therapy design produced by the method of claim 255 to help prevent, mitigate, delay, slow, reduce, mute, lessen, or treat an HBB-modulated recessive Sickle Cell disease, wherein the pathogenicity-specific supplemental polypeptide-inducing polynucleotide comprises the contiguous HBB promoter region (optionally minimal) and transcribable portion of a properly functional HBB gene, and the therapeutic protein is properly functional non-sickling hemoglobin subunit beta (HBB) protein.

257. (canceled)

258. A gene therapy design produced by the method of claim 255 to help prevent, mitigate, delay, slow, reduce, mute, lessen, or treat a GBA1-modulated recessive Gaucher disease, Parkinsonism, or familial tremor, wherein the pathogenicity-specific supplemental polypeptide-inducing polynucleotide comprises the contiguous GBA1 promoter region (optionally minimal) and transcribable portion of a properly functional GBA1 gene, and the therapeutic protein is properly functional non-pathogenic GBA1 glucocerebrosidase.

259. (canceled)

260. A gene therapy design produced by the method of claim 255 to help prevent, mitigate, delay, slow, reduce, mute, lessen, or treat a ASPA-modulated recessive Canavan disease, wherein the pathogenicity-specific supplemental polypeptide-inducing polynucleotide comprises the contiguous ASPA promoter region (optionally minimal) and transcribable portion of a properly functional ASPA gene, and the therapeutic protein is properly functional non-pathogenic ASPA Aspartoacylase.

261-262. (canceled)

263. A gene therapy design produced by the method of claim 255 to help prevent, mitigate, delay, slow, reduce, mute, lessen, or treat a SLC26A4-modulated recessive Pendred syndrome, wherein the pathogenicity-specific supplemental polypeptide-inducing polynucleotide comprises the contiguous SLC26A4 promoter region (optionally minimal) and transcribable portion of a properly functional SLC26A4 gene, and the therapeutic protein is properly functional non-pathogenic SLC26A4 Pendrin.

264. A gene therapy design produced by the method of claim 255 to help prevent, mitigate, delay, slow, reduce, mute, lessen, or treat a ACADM-modulated recessive Medium-chain acyl-CoA dehydrogenase deficiency, wherein the pathogenicity-specific supplemental polypeptide-inducing polynucleotide comprises the contiguous ACADM promoter region (optionally minimal) and transcribable portion of a properly functional ACADM gene, and the therapeutic protein is properly functional non-pathogenic ACADM medium chain acyl-CoA dehydrogenase.

265. A gene therapy design produced by the method of claim 255 to help prevent, mitigate, delay, slow, reduce, mute, lessen, or treat a CYP21A2-modulated recessive Congenital adrenal hyperplasia, wherein the pathogenicity-specific supplemental polypeptide-inducing polynucleotide comprises the contiguous CYP21A2 promoter region (optionally minimal) and transcribable portion of a properly functional CYP2142 gene, and the therapeutic protein is properly functional non-pathogenic CYP21A2 Steroid 21-hydroxylase.

266. A gene therapy design produced by the method of claim 255 to help prevent, mitigate, delay, slow, reduce, mute, lessen, or treat a ALDOB-modulated recessive Hereditary fructose intolerance, wherein the pathogenicity-specific supplemental polypeptide-inducing polynucleotide comprises the contiguous ALDOB promoter region (optionally minimal) and transcribable portion of a properly functional ALDOB gene, and the therapeutic protein is properly functional non-pathogenic ALDOB Aldolase B.

267-268. (canceled)

269. A gene therapy design produced by the method of claim 255 to help prevent, mitigate, delay, slow, reduce, mute, lessen, or treat a GAA-modulated recessive Pompe disease, wherein the pathogenicity-specific supplemental polypeptide-inducing polynucleotide comprises the contiguous GAA promoter region (optionally minimal) and transcribable portion of a properly functional GAA gene, and the therapeutic protein is properly functional non-pathogenic GAA Acid alpha-glucosidase.

270. A gene therapy design produced by the method of claim 255 to help prevent, mitigate, delay, slow, reduce, mute, lessen, or treat a GALT-modulated recessive Galactose-1-phosphate uridylyltransferase deficiency, wherein the pathogenicity-specific supplemental polypeptide-inducing polynucleotide comprises the contiguous GALT promoter region (optionally minimal) and transcribable portion of a properly functional GALT gene, and the therapeutic protein is properly functional non-pathogenic GALT Galactose-1-phosphate uridyltransferase.

271. A gene therapy design produced by the method of claim 255 to help prevent, mitigate, delay, slow, reduce, mute, lessen, or treat a FXN-modulated recessive Friedreich ataxia, wherein the pathogenicity-specific supplemental polypeptide-inducing polynucleotide comprises the contiguous FXN promoter region (optionally minimal) and transcribable portion of a properly functional FXN gene, and the therapeutic protein is properly functional non-pathogenic FXN Frataxin.

272. A gene therapy design produced by the method of claim 255 to help prevent, mitigate, delay, slow, reduce, mute, lessen, or treat a CYP21A2-modulated recessive Congenital adrenal hyperplasia due to 21-hydroxylase deficiency, wherein the pathogenicity-specific supplemental polypeptide-inducing polynucleotide comprises the contiguous CYP21A2 promoter region (optionally minimal) and transcribable portion of a properly functional CYP21A2 gene, and the therapeutic protein is properly functional non-pathogenic CYP21A2 Steroid 21-hydroxylase.

273. A gene therapy design produced by the method of claim 255 to help prevent, mitigate, delay, slow, reduce, mute, lessen, or treat a ADA-modulated recessive Severe combined immunodeficiency, wherein the pathogenicity-specific supplemental polypeptide-inducing polynucleotide comprises the contiguous ADA promoter region (optionally minimal) and transcribable portion of a properly functional ADA gene, and the therapeutic protein is properly functional non-pathogenic ADA Adenosine deaminase.

274. A gene therapy design produced by the method of claim 255 to help prevent, mitigate, delay, slow, reduce, mute, lessen, or treat a BTD-modulated recessive Biotinidase deficiency, wherein the pathogenicity-specific supplemental polypeptide-inducing polynucleotide comprises the contiguous BTD promoter region (optionally minimal) and transcribable portion of a properly functional BTD gene, and the therapeutic protein is properly functional non-pathogenic BTD Biotinidase.

275. (canceled)

276. A gene therapy design produced by the method of claim 255 to help prevent, mitigate, delay, slow, reduce, mute, lessen, or treat a ACADVL-modulated recessive Very-long-chain acyl-CoA dehydrogenase deficiency, wherein the pathogenicity-specific supplemental polypeptide-inducing polynucleotide comprises the contiguous ACADVL promoter region (optionally minimal) and transcribable portion of a properly functional ACADVL gene, and the therapeutic protein is properly functional non-pathogenic ACADVL Very long-chain specific acyl-CoA dehydrogenase.

277. A gene therapy design produced by the method of claim 255 to help prevent, mitigate, delay, slow, reduce, mute, lessen, or treat a GCDH-modulated recessive Glutaric acidemia type I, wherein the pathogenicity-specific supplemental polypeptide-inducing polynucleotide comprises the contiguous GCDH promoter region (optionally minimal) and transcribable portion of a properly functional GCDH gene, and the therapeutic protein is properly functional non-pathogenic GCDH Glutaryl-CoA dehydrogenase.

278. A gene therapy design produced by the method of claim 255 to help prevent, mitigate, delay, slow, reduce, mute, lessen, or treat a IDUA-modulated recessive Mucopolysaccharidosis type I, wherein the pathogenicity-specific supplemental polypeptide-inducing polynucleotide comprises the contiguous IDUA promoter region (optionally minimal) and transcribable portion of a properly functional IDUA gene, and the therapeutic protein is properly functional non-pathogenic IDUA Iduronidase.

279-280. (canceled)

281. A gene therapy design produced by the method of claim 255 to help prevent, mitigate, delay, slow, reduce, mute, lessen, or treat a CLN3-modulated recessive Juvenile neuronal ceroid lipofuscinosis, wherein the pathogenicity-specific supplemental polypeptide-inducing polynucleotide comprises the contiguous CLN3 promoter region (optionally minimal) and transcribable portion of a properly functional CLN3 gene, and the therapeutic protein is properly functional non-pathogenic CLN3 Battenin.

282. A gene therapy design produced by the method of claim 255 to help prevent, mitigate, delay, slow, reduce, mute, lessen, or treat a FAH-modulated recessive Tyrosinemia type I, wherein the pathogenicity-specific supplemental polypeptide-inducing polynucleotide comprises the contiguous FAH promoter region (optionally minimal) and transcribable portion of a properly functional FAH gene, and the therapeutic protein is properly functional non-pathogenic FAH Fumarylacetoacetate hydrolase.

283. (canceled)

284. A gene therapy design produced by the method of claim 255 to help prevent, mitigate, delay, slow, reduce, mute, lessen, or treat a PKLR-modulated recessive Pyruvate kinase deficiency, wherein the pathogenicity-specific supplemental polypeptide-inducing polynucleotide comprises the contiguous PKLR promoter region (optionally minimal) and transcribable portion of a properly functional PKLR gene, and the therapeutic protein is properly functional non-pathogenic Pyruvate kinase PKLR.

285. A gene therapy design produced by the method of claim 255 to help prevent, mitigate, delay, slow, reduce, mute, lessen, or treat a AGXT-modulated recessive Primary hyperoxaluria type 1, wherein the pathogenicity-specific supplemental polypeptide-inducing polynucleotide comprises the contiguous AGXT promoter region (optionally minimal) and transcribable portion of a properly functional AGXT gene, and the therapeutic protein is properly functional non-pathogenic AGXT Serine-pyruvate aminotransferase.

286. A gene therapy design produced by the method of claim 255 to help prevent, mitigate, delay, slow, reduce, mute, lessen, or treat a ASL-modulated recessive Argininosuccinic aciduria, wherein the pathogenicity-specific supplemental polypeptide-inducing polynucleotide comprises the contiguous ASL promoter region (optionally minimal) and transcribable portion of a properly functional ASL gene, and the therapeutic protein is properly functional non-pathogenic ASL argininosuccinate lyase.

287. A gene therapy design produced by the method of claim 255 to help prevent, mitigate, delay, slow, reduce, mute, lessen, or treat a CTNS-modulated recessive nephropathic Cystinosis, wherein the pathogenicity-specific supplemental polypeptide-inducing polynucleotide comprises the contiguous CTNS promoter region (optionally minimal) and transcribable portion of a properly functional CTNS gene, and the therapeutic protein is properly functional non-pathogenic CTNS cystinosin protein.

288. (canceled)

289. A gene therapy design produced by the method of claim 255 to help prevent, mitigate, delay, slow, reduce, mute, lessen, or treat a IVD-modulated recessive Isovaleric acidemia, wherein the pathogenicity-specific supplemental polypeptide-inducing polynucleotide comprises the contiguous IVD promoter region (optionally minimal) and transcribable portion of a properly functional IVD gene, and the therapeutic protein is properly functional non-pathogenic IVD isovaleryl-CoA dehydrogenase.

290. A gene therapy design produced by the method of claim 255 to help prevent, mitigate, delay, slow, reduce, mute, lessen, or treat a SMPD1-modulated recessive Niemann-Pick disease types A/B, wherein the pathogenicity-specific supplemental polypeptide-inducing polynucleotide comprises the contiguous SMPD1 promoter region (optionally minimal) and transcribable portion of a properly functional SMPD1 gene, and the therapeutic protein is properly functional non-pathogenic Sphingomyelin phosphodiesterase 1 (SMD1).

291. (canceled)

292. A gene therapy design produced by the method of claim 255 to help prevent, mitigate, delay, slow, reduce, mute, lessen, or treat a CBS-modulated recessive Homocystinuria, wherein the pathogenicity-specific supplemental polypeptide-inducing polynucleotide comprises the contiguous CBS promoter region (optionally minimal) and transcribable portion of a properly functional CBS gene, and the therapeutic protein is properly functional non-pathogenic CBS cystathione beta synthase.

293. A gene therapy design produced by the method of claim 255 to help prevent, mitigate, delay, slow, reduce, mute, lessen, or treat a HEXA-modulated recessive Tay-Sachs disease, wherein the pathogenicity-specific supplemental polypeptide-inducing polynucleotide comprises the contiguous HEXA promoter region (optionally minimal) and transcribable portion of a properly functional HEXA gene, and the therapeutic protein is properly functional non-pathogenic Hexosaminidase A (HEXA).

294. (canceled)

295. A gene therapy design produced by the method of claim 255 to help prevent, mitigate, delay, slow, reduce, mute, lessen, or treat a PPT1-modulated recessive Infantile neuronal ceroid lipofuscinoses, wherein the pathogenicity-specific supplemental polypeptide-inducing polynucleotide comprises the contiguous PPT1 promoter region (optionally minimal) and transcribable portion of a properly functional PPT1 gene, and the therapeutic protein is properly functional non-pathogenic Palmitoyl-protein thioesterase 1 (PPT1).

296. (canceled)

297. A gene therapy design produced by the method of claim 255 to help prevent, mitigate, delay, slow, reduce, mute, lessen, or treat a HEXB-modulated recessive Sandhoff disease, wherein the pathogenicity-specific supplemental polypeptide-inducing polynucleotide comprises the contiguous HEXB promoter region (optionally minimal) and transcribable portion of a properly functional HEXB gene, and the therapeutic protein is properly functional non-pathogenic HEXB Beta-hexosaminidase subunit beta.

298. A gene therapy design produced by the method of claim 255 to help prevent, mitigate, delay, slow, reduce, mute, lessen, or treat a MTTP-modulated recessive ABetalipoproteinemia, wherein the pathogenicity-specific supplemental polypeptide-inducing polynucleotide comprises the contiguous MTTP promoter region (optionally minimal) and transcribable portion of a properly functional MTTP gene, and the therapeutic protein is properly functional non-pathogenic MTTP Microsomal triglyceride transfer protein large subunit.

299-300. (canceled)

301. A gene therapy design produced by the method of claim 255 to help prevent, mitigate, delay, slow, reduce, mute, lessen, or treat a SMN1-modulated recessive Spinal muscular atrophy, wherein the pathogenicity-specific supplemental polypeptide-inducing polynucleotide comprises the contiguous SMN1 promoter region (optionally minimal) and transcribable portion of a properly functional SMN1 gene, and the therapeutic protein is properly functional non-pathogenic Survival of motor neuron 1 (SMN1).

302. A gene therapy design method, whereby the gene therapy helps prevent, mitigate, delay, slow, reduce, mute, lessen, or treat a target X-linked recessive genetic condition or disorder, comprising:
identifying a gene in which pathogenic variation is believed to modulate (be the root cause of) the target X-linked recessive genetic condition or disorder;
identifying the pathogenicity-specific supplemental polypeptide-inducing polynucleotide sequence comprising the contiguous sequence consisting of the promoter region (optionally minimal) and transcribable portion of a non-pathogenic functional version of the gene or generating a polynucleotide analogous thereto;
specifying or defaulting to a DNA structure, such as single-stranded or double-stranded linear DNA, plasmid DNA, DNA origami, 3DNA, or an analogue thereof, to comprise the polypeptide-inducing polynucleotide, wherein the transcribable structure is decorated with protein comprising a nuclear localization signal to facilitate subcellular localization for transcription, wherein optionally the promoter region of the polynucleotide is preceded in the DNA structure by a small molecule responsive element, such as TRE, for conditional transcription such as by tetracycline or doxycycline;
specifying or defaulting to a carrier to comprise the transcribable structure, or contain and release the transcribable structure, wherein the carrier is a lipid nanoparticle, a protein nanoparticle, DNA origami, another polynucleotide structure, or an analogue thereof, wherein the carrier is decorated by recipient-self-identifying protein, such as HLA protein autologous to the intended recipient, wherein the carrier is decorated by endocytosis-facilitating protein, such as clathrin protein;
wherein transcription of the transcribable portion of the polynucleotide in cell leads to production of mRNA with non-pathogenic wildtype subcellular localization capable of leading to translation of a therapeutic protein;

303. (canceled)

304. A gene therapy design method, whereby the gene therapy helps prevent, mitigate, delay, slow, reduce, mute, lessen, or treat at least one target autosomal dominant genetic condition or disorder, wherein neither moderate undersupply or moderate oversupply of the mediating genes nor moderate undersupply or moderate oversupply of the genes that they can mediate cause a worse pathogenic condition or disorder, comprising:
identifying a gene in which pathogenic variation is believed to carry the root cause of the target autosomal dominant genetic condition or disorder;
identifying non-pathogenic functional oligomers that comprise protein of a homologous non-pathogenic version of the identified gene;
identifying a non-pathogenic homologous version of each gene whose protein is part of an identified nonpathogenic functional oligomer;
wherein if the cause the pathogenic variation is believed to comprise active disruption, then identifying or generating a suppressive polynucleotide sequence for the detected pathogenic sequence, such as when in cell the pathogenic sequence leads to the production of protein that either (1) pathogenically interferes with the activity of another protein or (2) pathogenically interferes with the formation of a properly functional homodimer, heterodimer, or other oligomeric complex; and/or
wherein if the cause of a targeted dominant genetic condition comprises unbalanced co-expression, then identifying or generating a set of suppressive polynucleotide sequences that target transcripts of only one of the chromosomal haploids of each locus of protein that forms a non-pathogenic properly functional oligomer with protein of the locus pinpointed by the locus signature of the genetic condition, such that balanced co-expression ratios are restored among the non-pathogenic protein variants involved in proper formation of the non-pathogenic properly functional oligomers; and
wherein if one or more suppressive polynucleotides included in the design would cause haploinsufficiency or if the cause of a targeted dominant genetic condition or disorder comprises haploinsufficiency such as because there is insufficient quantity of properly functional protein, heterodimer, or other oligomeric complex, comprising protein induced by a non-pathogenic transcript variant homologous to the transcript with the pathogenic sequence variant, then for each gene necessary to be co-expressed to ensure expression level sufficiency while maintaining balanced co-expression, identifying the pathogenicity-specific supplemental polypeptide-inducing polynucleotide sequence comprising the contiguous sequence consisting of the promoter region (optionally minimal) and transcribable portion of a non-pathogenic functional version of the gene or generating a polynucleotide analogous thereto;
wherein if the gene therapy comprises a set of supplemental polypeptide-inducing polynucleotides, then the set of supplemental polypeptide-inducing polynucleotide is, or is part of, a (optionally conditionally) transcribable structure such as a DNA structure such as single-stranded or double-stranded linear DNA, plasmid DNA, DNA origami, 3DNA, or an analogue thereof, wherein in cell transcription of the transcribable structure is capable of leading to the production of therapeutic protein comprising the induced polypeptide producing transcripts from each supplemental polypeptide-inducing polynucleotide, with the respective non-pathogenic wildtype subcellular localization, capable of leading to translation of the respective induced therapeutic protein;
wherein if the gene therapy comprises a set of one or more suppressive polynucleotides, then each suppressive polynucleotide of the set is capable of being transcribed from (optionally conditionally), is part of, or is, a polynucleotide structure, wherein each of the polynucleotide structures comprise an ASO, an RNA structure, a DNA structure, or an analogue thereof;
wherein if the gene therapy comprises a (optionally conditionally) transcribable structure, then the transcribable structure is decorated with protein comprising a nuclear localization signal to facilitate subcellular localization for transcription, wherein optionally the promoter region of the polynucleotide is preceded in the transcribable structure by a small molecule responsive element, such as TRE, for conditional transcription such as by tetracycline or doxycycline;
wherein if the gene therapy is designed such that it substantively suppresses transcripts that originate from more than one chromosomal haploid at any single gene locus, then the gene therapy also comprises a supplemental polypeptide-inducing polynucleotide for a non-pathogenic homologous gene variant of the locus, and the suppressive polynucleotides and supplemental polynucleotides for the locus must be transcribable under the same general in cell conditions;
wherein if the gene therapy design comprises one or more supplemental polynucleotides together with one or more suppressive polynucleotides, then each supplemental and derivative RNA design is fortified against the overall set of suppressive polynucleotides;
specifying or defaulting to one or more carriers such as a lipid nanoparticle, a protein nanoparticle, DNA origami, or other structure comprise or is capable of containing and releasing one or more of the transcribable or polynucleotide structures, wherein the carrier is decorated by recipient-self-identifying protein, such as HLA protein autologous to the intended recipient, and decorated by endocytosis-facilitating protein such as clathrin protein;

305. A gene therapy design produced by the method of claim 304, wherein the gene therapy design helps prevent, mitigate, delay, slow, reduce, mute, lessen, or treat PC1 or PC2 modulated autosomal dominant polycystic kidney disease,
wherein non-pathogenic PC1 and PC2 form an non-pathogenic heterotetramer, wherein some PC1 and PC2 do not properly form a heterotetramer or do not form a properly functional heterotetramer, because either there is incompatibility between PC1 variant(s) and PC2 variant(s) induced by the PKD1 and PKD2 genes, or there is at least one pathogenic variation such as a so-called nonsense or missense mutation in either the PKD1 or PKD2 genes;
wherein the gene therapy design comprises a suppressive that targets transcripts of no more than one chromosomal haploid of the PKD1 gene locus and/or a suppressive that targets transcripts of no more than one chromosomal haploid of the PKD2 gene locus, such that the remaining PC1 variant(s) induced by PKD1 and the remaining PC2 variant(s) induced by PKD2 do properly oligomerize for proper function, wherein oligomerization between the untargeted variants of PC1 and the untargeted variants of PC2 do form exclusively non-pathogenic, properly functional heterotetramers.

306. A gene therapy design produced by the method of claim 304, wherein the gene therapy design helps prevent, mitigate, delay, slow, reduce, mute, lessen, or treat autosomal dominant MutLα-modulated Lynch syndrome wherein some MutLα do not properly form a dimer or do not form a properly functional dimer, because either there is incompatibility between MSH2 variant(s) and MSH6 variant(s) induced by the MSH2 and MSH6 genes, or there is at least one pathogenic variation such as a so-called nonsense or missense mutation in either the MSH2 or MSH6 genes;
wherein the gene therapy design comprises a suppressive that targets no more than one chromosomal haploid of the MSH2 gene locus and/or a suppressive that targets transcripts of no more than one chromosomal haploid of the MSH6 gene locus, such that the remaining MSH2 variant(s) induced by MSH2 and the remaining MSH6 variant(s) induced by MSH6 do properly dimerize to form a non-pathogenic MutLα that does properly function, wherein dimerization between the untargeted variants of MSH2 and the untargeted variants of MSH6 do form exclusively non-pathogenic, properly functional MutLα.

307. A gene therapy design produced by the method of claim 304, wherein the gene therapy design helps prevent, mitigate, delay, slow, reduce, mute, lessen, or treat autosomal dominant Li-Fraumeni syndrome wherein expressed TP53 variants do not properly tetramerize or do not form a properly functional tetramer, because either there is incompatibility between TP53 variant(s) induced by the TP53 gene locus, or there is at least one pathogenic variation such as a so-called nonsense or missense mutation in either chromosomal haploid of the TP53 gene;
wherein the gene therapy design comprises a suppressive that targets transcripts of no more than one chromosomal haploid of the TP53 gene locus, such that the remaining TP53 variant induced by TP53 does properly tetramerize for proper function, wherein all combinations of supplemented and untargeted variants of TP53 do form exclusively non-pathogenic, properly functional tetramers.

308. (canceled)

309. A gene therapy design produced by the method of claim 304, wherein the gene therapy design helps prevent, mitigate, delay, slow, reduce, mute, lessen, or treat autosomal dominant Familial Amyloid Polyneuropathy, Amyloidogenic Transthyretin Amyloidosis, and/or other TTR-modulated disease or disorder wherein expressed is a pathogenic TTR variant;
wherein the gene therapy design comprises a pathogenicity-specific suppressive that targets exclusively the pathogenic RNA(s) of no more than one chromosomal haploid of the TTR gene locus that produces the pathogenic TTR variant, by targeting a variation, that may be either considered pathogenic or benign, specific to the RNA(s) that produce the pathogenic TTR variant;
wherein optionally the gene therapy design may also comprise a pathogenicity-specific supplemental TTR.

310. An in silico method for designing a superior genome to improve crop performance of at least one phenotype with respect to a benchmark genome, comprising:
identifying a set of one or more genes from one or more reference genomes;
identifying a desired degree of increase, a desired degree of decrease, or a desired design level of expression to specify for each gene, homolog of the gene, or ortholog of the gene;
specifying an expression design level for each (sub) sequence of DNA to be generated, wherein transcription of DNA comprising the (sub) sequence altered expression design level leads to mRNA with half-life capable of implementing the desired degree of expression;
optionally specifying or defaulting to one or more desired resultant sequence properties or constraints under which sequence generation shall occur, directly or indirectly;
generating at least one resultant sequence for each gene of the set, wherein the superior genome design comprises a resultant sequence for each gene of the set, wherein the generation does not require but may optionally be enhanced by non-coding region design;
optionally generating one or more sequences of transformations capable of reifying the expression level designs; and
optionally implementing the superior genome by way of genetic engineering, wherein the difference between the superior genome and the benchmark genome is substantially equivalent to the set of genes and the changes therein.

311. A genome designed by the method of claim 310, wherein the set of genes comprises the USDA MaizeGDB Zm00001eb144510 gene (NCBI reference sequence: XP_008674696.1), a homolog thereof, or an ortholog thereof, wherein the generated resultant sequence may be generated under sequence and/or structure constraints that preserve subcellular localization and has an expression design level that differs from the (homologous) gene in the benchmark genome, wherein the sequence expression design level bestows a capability for increased seed number to increase yield potential or reduced seed number to accommodate limited resource conditions.

312. A genome designed by the method of claim 310, wherein the set of genes comprises the USDA MaizeGDB Zm00001eb396430 gene (NCBI reference sequence: XP_008660225.1), a homolog thereof, or an ortholog thereof, wherein the generated resultant sequence may be generated under sequence and/or structure constraints that preserve subcellular localization and has an expression design level that differs from the (homologous) gene in the benchmark genome, wherein the sequence expression design level bestows a capability for increased seed weight to increase yield potential or reduced seed weight for seed production efficiencies or distribution efficiencies.

313. A genome designed by the method of claim 310, wherein the set of genes comprises the USDA MaizeGDB Zm00001eb334460 gene (NCBI reference sequence: XP_008655551.1), a homolog thereof, or an ortholog thereof, wherein the generated resultant sequence may be generated under sequence and/or structure constraints that preserve subcellular localization and has an expression design level that differs from the (homologous) gene in the benchmark genome, wherein the sequence expression design level improves stalk strength to resist lodging, increases metabolic compatibility for animals and humans with inadequate or insufficient glucocerebrosidase, or increases biomass degradation to facilitate more efficient carbon capture for biofuel production.

314. A genome designed by the method of claim 310, wherein the set of genes comprises the USDA MaizeGDB Zm00001eb273450 gene (NCBI reference sequence: NP_001385188.1), a homolog thereof, or an ortholog thereof, wherein the generated resultant sequence may be generated under sequence and/or structure constraints that preserve subcellular localization and has an expression design level that differs from the (homologous) gene in the benchmark genome, wherein an increased sequence expression design level bestows a capability for increased insecticidal activity, against insect pests such as Lepidopteran or aphids to allow for increased yield potential.

315. A genome designed by the method of claim 310, wherein the set of genes comprises the USDA MaizeGDB Zm00001eb369850 gene (NCBI reference sequence: NP_001141223.1), a homolog thereof, or an ortholog thereof, wherein the generated resultant sequence may be generated under sequence and/or structure constraints that preserve subcellular localization and has an expression design level that differs from the (homologous) gene in the benchmark genome, wherein the sequence expression design level bestows a capability to adjust a florescence trait such as tassel branch number for improved production efficiency.

316. A genome designed by the method of claim 310, wherein the set of genes comprises the USDA MaizeGDB Zm00001eb337270 gene (NCBI gene symbol: LOC100384623), a homolog thereof, or an ortholog thereof, wherein the generated resultant sequence may be generated under sequence and/or structure constraints that preserve subcellular localization and has an expression design level that differs from the (homologous) gene in the benchmark genome, wherein an increased sequence expression design level bestows a capability for increased defense against pathogens such as Rhizoctonia solani in rice and Meloidogyne incognita in tomato to allow for increased yield potential.


SEQUENCE LISTING (148 sequences)

SEQ ID NO: 1 - protein, 463 aa, Zea mays
MRPRLPPNVPSLPSSLSLLANSLSSPVTNTPTRPDSFPAYLQLAHLMVSQERQEPAVPSSSSSSAKRAATSMDASPAPPLLLRAPTPSPSIDLPAGKDKADAAASKAGAAVFDLRREPKIPAPFLWPQEEARPSSAAELEVPMVDVGVLRNGDRAGLRRAAAQVAAACATHGFFQVCGHGVDAALGRAALDGASDFFRLPLAEKQRARRVPGTVSGYTSAHADRFAAKLPWKETLSFGYHDGAASPVVVDYFVGTLGQDFEPMGWVYQRYCEEMKELSLTIMELLELSLGVELRGYYREFFEDSRSIMRCNYYPPCPEPERTLGTGPHCDPTALTILLQDDVGGLEVLVDGEWRPVRPVPGAMVINIGDTFMALSNGRYKSCLHRAVVNQRRARRSLAFFLCPREDRVVRPPASAAPRRYPDFTWADLMRFTQRHYRADTRTLDAFTRWLSHGPAQAAAPPCT

SEQ ID NO: 2 - RNA, 1392 nt, synthetic construct
  source 1..1392: mol_type = other RNA
atgcgcccccggctcccgccgaacgtgccgtcactccccagctccttgagtctcttggcgaactccctgagtagccccgtgaccaacaccccgactcggcccgacagcttccctgcatacttgcagctggctcacctaatggtgagccaggagcggcaggagccggcggtgccgtccagctcgtcgtcgtccgcaaagcgggcggcgacgagcatggacgcatcgccggctccgccgctcctgctgcgtgcacccaccccatcgccctcgatagacctgcccgcgggtaaagacaaagcggacgccgccgcctccaaagcgggggcggcggtgttcgacctgcggcgtgagccgaagatcccggcaccgttcctctggccccaggaggaggcgcgaccctcgtcggccgcagagctggaagtgcccatggtggacgtaggcgtgctgcgaaatggggaccgcgcggggctacgccgcgcggctgcccaggtcgcagccgcctgcgccacccatgggttcttccaggtctgcggccacggggtcgacgccgccctggggcgagcggcactcgacggggcctctgacttcttccgcctacctttggcggagaagcagagggcccgtcgagtgccgggaacggtgtcgggatatacttcggctcacgccgacaggttcgcggcaaaactgccgtggaaggagacactctccttcggctaccacgacggggcggctagccccgtcgtggtagactactttgtcggtaccctcgggcaggacttcgagccgatggggtgggtgtaccagcggtattgtgaggagatgaaagaattatccctcacaataatggagctgttggagctgagtctgggtgttgagttacggggctactacagggagttcttcgaggactcaaggagcataatgcggtgtaattattatcccccgtgcccagagccggaacggactctgggcacggggccgcattgtgatccaacagcactgacaatcctcctacaggacgatgtaggaggattggaggtgctggtggatggggagtggcggcccgttcggccggtgccgggggcgatggtcataaacattggagacacgtttatggccctgagtaatgggcggtataaaagctgcctgcaccgggccgtggtcaaccagcgccgagcgcgtcggagcctggcgttcttcctgtgtccgcgggaggaccgcgtggtccgccctccggctagcgccgcaccgcggcgctatccggattttacttgggcggacctaatgcggttcacccagcggcactacagggcggacaccaggactctcgacgcgttcacgcgctggttgagccacggcccggcgcaggcagctgcgccgccctgtacttag

SEQ ID NO: 3 - RNA, 1392 nt, synthetic construct
  source 1..1392: mol_type = other RNA
atgagaccaagattaccaccaaatgtaccatcattaccatcatcattatcattattagcaaattcattatcatcaccagtaacaaatacaccaacaagaccagattcatttccagcatatttacaattagcacatttaatggtatcacaagaaagacaagaaccagcagtaccatcatcatcatcatcatcagcaaaaagagcagcaacatcaatggacgcatcaccagcaccaccattattattaagagcaccaacaccatcaccatcaatagatttaccagcaggaaaagataaagcagatgcagcagcatcaaaagcaggagcagcagtatttgatttaagaagagaaccaaaaataccagcaccatttttatggccacaagaagaagcaagaccatcatcagcagcagaattagaagtaccaatggtagatgtaggagtattaagaaatggagatagagcaggattaagaagagcagcagcacaagtagcagcagcatgtgcaacacatggattttttcaagtatgcggacatggagtagatgcagcattaggaagagcagcattagacggagcatcagatttttttagattaccattagcagaaaaacaaagagcaagaagagtaccaggaacagtatcaggatatacatcagcacatgcagatagatttgcagcaaaattaccatggaaagaaacattatcatttggatatcatgacggagcagcatcaccagtagtagtagattattttgtaggaacattaggacaagattttgaaccaatgggatgggtatatcaaagatattgtgaagaaatgaaagaattatcattaacaataatggaattattagaattatcattaggagtagaattaagaggatattatagagaattttttgaagattcaagatcaataatgagatgtaattattatccaccatgtccagaaccagaaagaacattaggaacaggaccacattgcgatccaacagcattaacaatattattacaagatgatgtaggaggactagaagtattagtagacggagaatggagaccagtaagaccagtaccaggagcaatggtaataaatataggagatacatttatggcattatcaaatggaagatataaatcatgtttacatagagcagtagtaaatcaaagaagagcaagaagatcattagcattttttttatgtccaagagaagatagagtagtaagaccaccagcatcagcagcaccaagaagatatccagattttacatgggcagatttaatgagatttacacaaagacattatagagcagatacaagaacattagatgcatttacaagatggttatcacatggaccagcacaagcagcagcaccaccatgtacataa

SEQ ID NO: 4 - RNA, 1392 nt, Zea mays
  source 1..1392: mol_type = other RNA
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 5 - RNA, 1392 nt, synthetic construct
  source 1..1392: mol_type = other RNA
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcagtagtaaatcaaagaagagcaagaagatcattagcattttttttatgtccaagagaagatagagtagtaagaccaccagcatcagcagcaccaagaagatatccagattttacatgggcagatttaatgagatttacacaaagacattatagagcagatacaagaacattagatgcatttacaagatggttatcacatggaccagcacaagcagcagcaccaccatgtacataa

SEQ ID NO: 6 - RNA, 1392 nt, synthetic construct
  source 1..1392: mol_type = other RNA
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcacagagcagtagtaaatcaaagaagagcaagaagatcattagcattttttttatgtccaagagaagatagagtagtaagaccaccagcatcagcagcaccaagaagatatccagattttacatgggcagatttaatgagatttacacaaagacattatagagcagatacaagaacattagatgcatttacaagatggttatcacatggaccagcacaagcagcagcaccaccatgtacataa

SEQ ID NO: 7 - DNA, 2841 bp, Zea mays
  source 1..2841: mol_type = other DNA
tgtcgggtcccacatgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaactgatttctgtgaa

SEQ ID NO: 8 - RNA, 2841 nt, synthetic construct
  source 1..2841: mol_type = other RNA
tgtcgggtcccacatgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcagtagtaaatcaaagaagagcaagaagatcattagcattttttttatgtccaagagaagatagagtagtaagaccaccagcatcagcagcaccaagaagatatccagattttacatgggcagatttaatgagatttacacaaagacattatagagcagatacaagaacattagatgcatttacaagatggttatcacatggaccagcacaagcagcagcaccaccatgtacataacgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaactgatttctgtgaa

SEQ ID NO: 9 - protein, 653 aa, Zea mays
MANPNNRSEHDTIKVTPNSELQTNHNQYPLADNPNSTLEELNYKEFLRMTEDSSTEVLDNSTVKDAVGTGISVVGQILGVVGVPFAGALTSFYQSFLNTIWPSDADPWKAFMAQVEVLIDKKIEEYAKSKALAELQGLQNNFEDYVNALNSWKKTPLSLRSKRSQGRIRELFSQAESHFRNSMPSFAVSKFEVLFLPTYAQAANTHLLLLKDAQVFGEEWGYSSEDVAEFYRRQLKLTQQYTDHCVNWYNVGLNGLRGSTYDAWVKFNRFRREMTLTVLDLIVLFPFYDIRLYSKGVKTELTRDIFTDPIFLLTTLQKYGPTFLSIENSIRKPHLFDYLQGIEFHTRLRPGYFGKDSFNYWSGNYVETRPSIGSSKTITSPFYGDKSTEPVQKLSFDGQKVYRTIANTDVAAWPNGKVYLGVTKVDFSQYDDQKNETSTQTYDSKRNNGHVSAQDSIDQLPPETTDEPLEKAYSHQLNYAECFLMQDRRGTIPFFTWTHRSVDFFNTIDAEKITQLPVVKAYALSSGASIIEGPGFTGGNLLFLKESSNSIAKFKVTLNSAALLQRYRVRIRYASTTNLRLFVQNSNNDFLVIYINKTMNKDDDLTYQTFDLATTNSNMGFSGDKNELIIGAESFVSNEKIYIDKIEFIPVQL

SEQ ID NO: 10 - RNA, 1962 nt, synthetic construct
  source 1..1962: mol_type = other RNA
atggcgaatccgaacaaccggtctgagcatgatacgatcaaagtgactccgaactcggagttacagacgaatcataatcagtacccgttggcggataatcccaacagcacactggaagaactcaattataaggagttcttacgaatgaccgaagattcgtcgaccgaggtcctggataactccaccgtgaaagacgcggtggggaccgggatctcggttgtcggtcagatcctcggcgtggtcggagtgcctttcgcgggtgcgctaacgtcgttctaccagtcgtttttaaatacgatctggccgagcgacgcggacccgtggaaggcattcatggcccaggtcgaggttctgatcgacaagaagatagaggagtatgccaagagcaaggcgttggcggagcttcaaggtttacaaaataactttgaagactacgtcaacgccttgaactcttggaaaaagactcctctatctttgcggagtaagcgcagtcagggccggatccgtgagctcttctcccaggcggaatcgcattttcggaactcgatgccaagcttcgctgtgtcgaagttcgaagtcctgttcttgcccacgtatgcccaagcggcgaatacccacctactcctcctcaaagacgcacaggtctttggggaggagtgggggtattcgtcggaggatgtcgccgagttctatcggcgacaactcaagttgacgcagcagtacacagaccactgcgtcaactggtataacgtgggcttgaacggacttcgaggttcgacatacgatgcttgggtcaagttcaatcgattccgccgggagatgacgctcacggttctggacctgattgtgctttttccgttttacgacatccgtctttactccaaaggagtgaagacggagttgactcgcgatatctttacggacccgatatttctgctaacaacgctgcagaaatatgggccgactttcttatcgattgaaaattcgataagaaagccccatttgttcgactaccttcagggcatcgagttccacacccggttgaggcccgggtacttcggtaaagatagcttcaactactggtcggggaactacgtggaaacgaggccctcgatcggtagttcgaagactatcacgtctccgttttacggcgacaagtcgacggagcctgtgcagaagctatcgttcgatggacaaaaagtctatcgaacgatagctaatactgacgtggccgcttggccgaatggaaaggtatatctgggtgtgacgaaagtcgacttcagtcagtacgatgaccagaagaacgagacgtccacccagacgtacgattccaagcggaacaacggccacgtctctgcacaggactccatcgaccagttgccgccggagacgacggacgagccgctggagaaggcgtactctcaccagctgaattatgctgagtgttttctcatgcaggataggagggggacaatccccttcttcacctggacgcatcgatcggttgattttttcaacacgatcgatgcggagaagatcactcagcttccggtggtgaaggcgtacgccttgtccagcggcgcgtccattatcgaaggcccgggcttcaccggggggaacttgttgttcctgaaggagtcgagcaacagcatcgccaaatttaaggtgacgctgaattctgcagcgttgttgcagagatatcgggtccgtataagatacgcgagtacgacgaatcttcggttattcgtgcagaactccaataatgattttctcgttatttacattaataaaactatgaataaggacgacgacctcacttatcaaacgttcgatcttgccaccacgaactccaacatggggttcagtggtgacaagaacgaactgataattggggcggagtcctttgtgagtaacgagaagatctatattgataagattgagttcattccagtgcagctgtaa

SEQ ID NO: 11 - RNA, 1962 nt, synthetic construct
  source 1..1962: mol_type = other RNA
atggcaaacccaaacaacagatcagaacacgacacaataaaagtaacaccaaactcagaactacaaacaaaccacaaccaatacccactagcagacaacccaaactcaacactagaagaactaaactacaaagaattcctaagaatgacagaagactcatcaacagaagtactagacaactcaacagtaaaagacgcagtaggaacaggaatatcagtagtaggacaaatactaggagtagtaggagtaccattcgcaggagcactaacatcattctaccaatcattcctaaacacaatatggccatcagacgcagacccatggaaagcattcatggcacaagtagaagtactaatagacaaaaaaatagaagaatacgcaaaatcaaaagcactagcagaactacaaggactacaaaacaacttcgaagactacgtaaacgcactaaactcatggaaaaaaacaccactatcactaagatcaaaaagatcacaaggaagaataagagaactattctcacaagcagaatcacacttcagaaactcaatgccatcattcgcagtatcaaaattcgaagtactattcctaccaacatacgcacaagcagcaaacacacacctactactactaaaagacgcacaagtattcggagaagaatggggatactcatcagaagacgtagcagaattctacagaagacaactaaaactaacacaacaatacacagaccactgcgtaaactggtacaacgtaggactaaacggactaagaggatcaacatacgacgcatgggtaaaattcaacagattcagaagagaaatgacactaacagtactagacctaatagtactattcccattctacgacataagactatactcaaaaggagtaaaaacagaactaacaagagacatattcacagacccaatattcctactaacaacactacaaaaatacggaccaacattcctatcaatagaaaactcaataagaaaaccacacctattcgactacctacaaggaatagaattccacacaagactaagaccaggatacttcggaaaagactcattcaactactggtcaggaaactacgtagaaacaagaccatcaataggatcatcaaaaacaataacatcaccattctacggagacaaatcaacagaaccagtacaaaaactatcattcgacggacaaaaagtatacagaacaatagcaaacacagacgtagcagcatggccaaacggaaaagtatacctaggagtaacaaaagtagacttctcacaatacgacgaccaaaaaaacgaaacatcaacacaaacatacgactcaaaaagaaacaacggacacgtatcagcacaagactcaatagaccaactaccaccagaaacaacagacgaaccactagaaaaagcatactcacaccaactaaactacgcagaatgcttcctaatgcaagacagaagaggaacaataccattcttcacatggacacacagatcagtagacttcttcaacacaatagacgcagaaaaaataacacaactaccagtagtaaaagcatacgcactatcatcaggagcatcaataatagaaggaccaggattcacaggaggaaacctactattcctaaaagaatcatcaaactcaatagcaaaattcaaagtaacactaaactcagcagcactactacaaagatacagagtaagaataagatacgcatcaacaacaaacctaagactattcgtacaaaactcaaacaacgacttcctagtaatatacataaacaaaacaatgaacaaagacgacgacctaacataccaaacattcgacctagcaacaacaaactcaaacatgggattctcaggagacaaaaacgaactaataataggagcagaatcattcgtatcaaacgaaaaaatatacatagacaaaatagaattcataccagtacaactataa

SEQ ID NO: 12 - RNA, 1962 nt, synthetic construct
  source 1..1962: mol_type = other RNA
atggcgaatccgaacaaccggtctgagcatgatacgatcaaagtgactccgaactcggagttacagacgaatcataatcagtacccgttggcggataatcccaacagcacactggaagaactcaattataaggagttcttacgaatgaccgaagattcgtcgaccgaggtcctggataactccaccgtgaaagacgcggtggggaccgggatctcggttgtcggtcagatcctcggcgtggtcggagtgcctttcgcgggtgcgctaacgtcgttctaccagtcgtttttaaatacgatctggccgagcgacgcggacccgtggaaggcattcatggcccaggtcgaggttctgatcgacaagaagatagaggagtatgccaagagcaaggcgttggcggagcttcaaggtttacaaaataactttgaagactacgtcaacgccttgaactcttggaaaaagactcctctatctttgcggagtaagcgcagtcagggccggatccgtgagctcttctcccaggcggaatcgcattttcggaactcgatgccaagcttcgctgtgtcgaagttcgaagtcctgttcttgcccacgtatgcccaagcggcgaatacccacctactcctcctcaaagacgcacaggtctttggggaggagtgggggtattcgtcggaggatgtcgccgagttctatcggcgacaactcaagttgacgcagcagtacacagaccactgcgtcaactggtataacgtgggcttgaacggacttcgaggttcgacatacgatgcttgggtcaagttcaatcgattccgccgggagatgacgctcacggttctggacctgattgtgctttttccgttttacgacatccgtctttactccaaaggagtgaagacggagttgactcgcgatatctttacggacccgatatttctgctaacaacgctgcagaaatatgggccgactttcttatcgattgaaaattcgataagaaagccccatttgttcgactaccttcagggcatcgagttccacacccggttgaggcccgggtacttcggtaaagatagcttcaactactggtcggggaactacgtggaaacgaggccctcgatcggtagttcgaagactatcacgtctccgttttacggcgacaagtcgacggagcctgtgcagaagctatcgttcgatggacaaaaagtctatcgaacgatagctaatactgacgtggccgcttggccgaatggaaaggtatatctgggtgtgacgaaagtcgacttcagtcagtacgatgaccagaagaacgagacgtccacccagacgtacgattccaagcggaacaacggccacgtatcagcacaagactcaatagaccaactaccaccagaaacaacagacgaaccactagaaaaagcatactcacaccaactaaactacgcagaatgcttcctaatgcaagacagaagaggaacaataccattcttcacatggacacacagatcagtagacttcttcaacacaatagacgcagaaaaaataacacaactaccagtagtaaaagcatacgcactatcatcaggagcatcaataatagaaggaccaggattcacaggaggaaacctactattcctaaaagaatcatcaaactcaatagcaaaattcaaagtaacactaaactcagcagcactactacaaagatacagagtaagaataagatacgcatcaacaacaaacctaagactattcgtacaaaactcaaacaacgacttcctagtaatatacataaacaaaacaatgaacaaagacgacgacctaacataccaaacattcgacctagcaacaacaaactcaaacatgggattctcaggagacaaaaacgaactaataataggagcagaatcattcgtatcaaacgaaaaaatatacatagacaaaatagaattcataccagtacaactataa

SEQ ID NO: 13 - RNA, 1962 nt, synthetic construct
  source 1..1962: mol_type = other RNA
atggcgaatccgaacaaccggtctgagcatgatacgatcaaagtgactccgaactcggagttacagacgaatcataatcagtacccgttggcggataatcccaacagcacactggaagaactcaattataaggagttcttacgaatgaccgaagattcgtcgaccgaggtcctggataactccaccgtgaaagacgcggtggggaccgggatctcggttgtcggtcagatcctcggcgtggtcggagtgcctttcgcgggtgcgctaacgtcgttctaccagtcgtttttaaatacgatctggccgagcgacgcggacccgtggaaggcattcatggcccaggtcgaggttctgatcgacaagaagatagaggagtatgccaagagcaaggcgttggcggagcttcaaggtttacaaaataactttgaagactacgtcaacgccttgaactcttggaaaaagactcctctatctttgcggagtaagcgcagtcagggccggatccgtgagctcttctcccaggcggaatcgcattttcggaactcgatgccaagcttcgctgtgtcgaagttcgaagtcctgttcttgcccacgtatgcccaagcggcgaatacccacctactcctcctcaaagacgcacaggtctttggggaggagtgggggtattcgtcggaggatgtcgccgagttctatcggcgacaactcaagttgacgcagcagtacacagaccactgcgtcaactggtataacgtgggcttgaacggacttcgaggttcgacatacgatgcttgggtcaagttcaatcgattccgccgggagatgacgctcacggttctggacctgattgtgctttttccgttttacgacatccgtctttactccaaaggagtgaagacggagttgactcgcgatatctttacggacccgatatttctgctaacaacgctgcagaaatatgggccgactttcttatcgattgaaaattcgataagaaagccccatttgttcgactaccttcagggcatcgagttccacacccggttgaggcccgggtacttcggtaaagatagcttcaactactggtcggggaactacgtggaaacgaggccctcgatcggtagttcaaaaacaataacatcaccattctacggagacaaatcaacagaaccagtacaaaaactatcattcgacggacaaaaagtatacagaacaatagcaaacacagacgtagcagcatggccaaacggaaaagtatacctaggagtaacaaaagtagacttctcacaatacgacgaccaaaaaaacgaaacatcaacacaaacatacgactcaaaaagaaacaacggacacgtatcagcacaagactcaatagaccaactaccaccagaaacaacagacgaaccactagaaaaagcatactcacaccaactaaactacgcagaatgcttcctaatgcaagacagaagaggaacaataccattcttcacatggacacacagatcagtagacttcttcaacacaatagacgcagaaaaaataacacaactaccagtagtaaaagcatacgcactatcatcaggagcatcaataatagaaggaccaggattcacaggaggaaacctactattcctaaaagaatcatcaaactcaatagcaaaattcaaagtaacactaaactcagcagcactactacaaagatacagagtaagaataagatacgcatcaacaacaaacctaagactattcgtacaaaactcaaacaacgacttcctagtaatatacataaacaaaacaatgaacaaagacgacgacctaacataccaaacattcgacctagcaacaacaaactcaaacatgggattctcaggagacaaaaacgaactaataataggagcagaatcattcgtatcaaacgaaaaaatatacatagacaaaatagaattcataccagtacaactataa

SEQ ID NO: 14 - RNA, 1962 nt, synthetic construct
  source 1..1962: mol_type = other RNA
atggcgaatccgaacaaccggtctgagcatgatacgatcaaagtgactccgaactcggagttacagacgaatcataatcagtacccgttggcggataatcccaacagcacactggaagaactcaattataaggagttcttacgaatgaccgaagattcgtcgaccgaggtcctggataactccaccgtgaaagacgcggtggggaccgggatctcggttgtcggtcagatcctcggcgtggtcggagtgcctttcgcgggtgcgctaacgtcgttctaccagtcgtttttaaatacgatctggccgagcgacgcggacccgtggaaggcattcatggcccaggtcgaggttctgatcgacaagaagatagaggagtatgccaagagcaaggcgttggcggagcttcaaggtttacaaaataactttgaagactacgtcaacgccttgaactcttggaaaaagactcctctatctttgcggagtaagcgcagtcagggccggatccgtgagctcttctcccaggcggaatcgcattttcggaactcgatgccaagcttcgctgtgtcgaagttcgaagtcctgttcttgcccacgtatgcccaagcggcgaatacccacctactcctcctcaaagacgcacaggtctttggggaggagtgggggtattcgtcggaggatgtcgccgagttctatcggcgacaactcaagttgacgcagcagtacacagaccactgcgtcaactggtataacgtgggcttgaacggacttcgaggttcgacatacgatgcttgggtcaagttcaatcgattccgccgggagatgacgctcacggttctggacctgatagtactattcccattctacgacataagactatactcaaaaggagtaaaaacagaactaacaagagacatattcacagacccaatattcctactaacaacactacaaaaatacggaccaacattcctatcaatagaaaactcaataagaaaaccacacctattcgactacctacaaggaatagaattccacacaagactaagaccaggatacttcggaaaagactcattcaactactggtcaggaaactacgtagaaacaagaccatcaataggatcatcaaaaacaataacatcaccattctacggagacaaatcaacagaaccagtacaaaaactatcattcgacggacaaaaagtatacagaacaatagcaaacacagacgtagcagcatggccaaacggaaaagtatacctaggagtaacaaaagtagacttctcacaatacgacgaccaaaaaaacgaaacatcaacacaaacatacgactcaaaaagaaacaacggacacgtatcagcacaagactcaatagaccaactaccaccagaaacaacagacgaaccactagaaaaagcatactcacaccaactaaactacgcagaatgcttcctaatgcaagacagaagaggaacaataccattcttcacatggacacacagatcagtagacttcttcaacacaatagacgcagaaaaaataacacaactaccagtagtaaaagcatacgcactatcatcaggagcatcaataatagaaggaccaggattcacaggaggaaacctactattcctaaaagaatcatcaaactcaatagcaaaattcaaagtaacactaaactcagcagcactactacaaagatacagagtaagaataagatacgcatcaacaacaaacctaagactattcgtacaaaactcaaacaacgacttcctagtaatatacataaacaaaacaatgaacaaagacgacgacctaacataccaaacattcgacctagcaacaacaaactcaaacatgggattctcaggagacaaaaacgaactaataataggagcagaatcattcgtatcaaacgaaaaaatatacatagacaaaatagaattcataccagtacaactataa

SEQ ID NO: 15 - DNA, 1885 bp, Zea mays
  source 1..1885: mol_type = other DNA
acgaaccccaccttcttcgttgcctctcttcctccctctcctctcttccttcctctcgatcgatcgtcatagcaatccaagctagcaagcccgtccgtcgtctctcgtccaagaagcatacaactactaatggggaggtcaccgtgctgcgaccaggacgcgggcgtcaagaaaggcccgtggacgccggaggaggacaagctgctggtggactacatcaacggcaacggccacgggagctggcgccgcctgcccaagcacgccggcctcaaccgctgcggcaagagctgccgcctccgctggaccaactacctccgcccggacatcaagcgcggccgcttcaccgacgacgaggagaagctcatcatccacctccactccctcctcggcaacaagtggtccgccatcgccaccaagctgcccggccggaccgacaacgagatcaagaactactggaacacgcacctccgcaagaagctgctcgccatggggatcgaccccgtcacgcaccagcgccgcaccgacctcaacctcctcgcggccggcggcttcaccaacctcctcgccgccgccaacctcgcggccgccgccgctggtggcggccaagcagctggacctggaggaccgctcgccgccgcgcatgctagctcctgctgggacatcaacgctctcaggctccaggccgacgccgccaagtaccagctcctcgagggcctcgtccgcgtgctcaccgcccccgccgcgcccaccgtcgacctcatgtccctcctcgccgccgccaataatggaggcgcgcccagcggcggcggcggccaccagcagctgctcgccggagtggaccaaagcaccgccagcagggtcgtgcagcagtacgacggcatgctcagccttccagcgctgaccagcgccgtgccggcggtgacgcagggcatgccgtcctcctccgcctactccctctctgggctcctcaacggcttctccggcggtgccgggactacggccgctttcgccggggacgggctcagctccacggagctcggccacagcggggccaccaccgggagcaacgtgactgccgccgcgatgccgccacctttggcagcggcggccaaggggtgcagtgcaggcaacggcggcgccacgtcgtcgaccccatccccgtacgaggagacgccggcgtcgagcccgttcgacggcctggacagcctcacgctggattttgaccccaacaacgacagttggagagaattgctagagtgagtataccacctatagcatcgatctagcataccatgccatgcatgcatcaactgtagtgtttgcttatgtatattttctagctacaaagcagtatacatgcatgtaaattatatacatactagctagactaagattcaaaaaaaaaaaaaaaactttagcgtactaaatagcatgtgtgtagatacatatcctgatttaatttgctattagctacaggtatagatgtatgtaagttatatatatatatatatatatatatatatatatatatatatatatatatatatatatatatatatatatatatatatatatatatatatatatatatagactagatgctataagattccaaaaaaataaaactttagcttactaaatatcatgtgtgtatatatatccttgtttaatctgccagctaaaagtatatatgcatgtaaatatggatatactacctagctatgatttccaaaaaaaaggcttagcttacgaaatagtaacatgtatatagtatttattttcttattgttgcctctgatctcaccatgtattttccttctctttgtcctttggtgcatgtgcaggcaaatgtcatggttgaacaaccccaatgatcagctgtgag

SEQ ID NO: 16 - DNA, 1885 bp, synthetic construct
  source 1..1885: mol_type = other DNA
acgaaccccaccttcttcgttgcctctcttcctccctctcctctcttccttcctctcgatcgatcgtcatagcaatccaagctagcaagcccgtccgtcgtctctcgtccaagaagcatacaactactaatgggtcgcagcccatgctgcgaccaagatgccggcgtgaagaaggggccgtggactcccgaggaagataagctgttggtggattacatcaacggcaacggccacgggagttggcggcgccttcctaagcacgccggcctcaatcggtgcggcaagagctgtcggttgcgatggaccaactacttgcggcccgacattaagcgagggaggtttacggatgatgaggaaaaactcatcatccacctccactcgctgttgggcaacaagtggtcggccatcgcaaccaagctccctggccgcaccgacaacgagatcaagaattattggaacacgcatctgcgcaagaagctcctcgccatgggcattgatccggtcactcaccagcgtcggactgacctcaacctcctcgcggccggcggcttcaccaacctcctcgccgccgccaacctcgcggccgccgccgctggtggcggccaagcagctggacctggaggaccgctcgccgccgcgcatgctagctcctgctgggacatcaacgctctcaggctccaggccgacgccgccaagtaccagctcctcgagggcctcgtccgcgtgctcaccgcccccgccgcgcccaccgtcgacctcatgtccctcctcgccgccgccaataatggaggcgcgcccagcggcggcggcggccaccagcagctgctcgccggagtggaccaaagcaccgccagcagggtcgtgcagcagtacgacggcatgctcagccttccagcgctgaccagcgccgtgccggcggtgacgcagggcatgccgtcctcctccgcctactccctctctgggctcctcaacggcttctccggcggtgccgggactacggccgctttcgccggggacgggctcagctccacggagctcggccacagcggggccaccaccgggagcaacgtgactgccgccgcgatgccgccacctttggcagcggcggccaaggggtgcagtgcaggcaacggcggcgccacgtcgtcgaccccatccccgtacgaggagacgccggcgtcgagcccgttcgacggcctggacagcctcacgctggattttgaccccaacaacgacagttggagagaattgctagagtgagtataccacctatagcatcgatctagcataccatgccatgcatgcatcaactgtagtgtttgcttatgtatattttctagctacaaagcagtatacatgcatgtaaattatatacatactagctagactaagattcaaaaaaaaaaaaaaaactttagcgtactaaatagcatgtgtgtagatacatatcctgatttaatttgctattagctacaggtatagatgtatgtaagttatatatatatatatatatatatatatatatatatatatatatatatatatatatatatatatatatatatatatatatatatatatatatatatatagactagatgctataagattccaaaaaaataaaactttagcttactaaatatcatgtgtgtatatatatccttgtttaatctgccagctaaaagtatatatgcatgtaaatatggatatactacctagctatgatttccaaaaaaaaggcttagcttacgaaatagtaacatgtatatagtatttattttcttattgttgcctctgatctcaccatgtattttccttctctttgtcctttggtgcatgtgcaggcaaatgtcatggttgaacaaccccaatgatcagctgtgag

SEQ ID NO: 17 - DNA, 4039 bp, Zea mays
  source 1..4039: mol_type = other DNA
ccaaaagccggccacttacttgcgtctgcataatactacaaggcatccggcctatatgccgggtgatcgatgatgatgatgatataaaactccgaggaccaaaaaagaaaaaaccgtacgttaccaattaagagcttttgctttcaagaagaatttctctatacaggcgtgcatggtgggtgtttgatttcatttttggaacgtaatggtgtttgatttctagcacagacagagacagagggtgtggcgttacacatctctgcctatatataaacaggaggcgttgcgtgcagagctgccggctggcggttgcctctcctcccctcccctgccctcgcaaacgcacgcaccacagcatcgtccttcgttccattccggcattcccatccatataccccctcgatcggccgggatgctgggtgtggggatggccgcggcggtgctgctcggggccgtggcgttgctgctcgcggacgccgctgcgaggagagcgcactggtggtacagggaggcggctgaggcggtgctggtcggcgccgtggctttggtggtggtggacgccgcggcgcggagggcgcacgggtggtacagggaagcggcgctgggcgcggcgcggcgggcgcggctgccgccgggggagatggggtggcccttggtcggcggcatgtgggccttcctccgcgccttcaagtccggcaagcccgacgccttcatcgcctccttcgtccggcggtgagtcagtgcgctaatcctcgatctcctccctcctcccctgcacgctcgctccgctctgctcctaccgaggatgggccgggccccacgtgtcattgtcctcgtcgacacctgcacctgcacgcaccgggccccacggccgtgtgagcttgtggcgcatgggtgcaccggagcagcattagctagccagcagcaatggatagctgtcgattttctatcccaggctggttaattaatcctcatccctttgcctgctaaataggagtattacttgtactgaaatcaccaaaatctagaacataaccgttatgacagctgtatatatagaatatgaatagttaaatggctcgtcaagccaaattttatctaatcaacagcaaaataactacatatatacaacaaagtaaccatctaaataactcatcaagctatttgtctctttaaattggctctccctcacaggccaaacttgactagctaaactagatagatagtctattggattgagatgttacacatagaatgtaatctttatagaaaagtaaatagatagtcaaataaagagaaaaaaaatgaagagtactgtgaagatgctctactagtagtaccagaaattaagaagtatatataactcacataggcatgcattgcacttgtagtcgcaagcaattcactgctgttacttttgtaaacgaagcagtcagacaggagagctatcgagtatcaaaagaaggccagaccagactgagacagggtagaacgacggtcgacgacgacgtcccgcgcgcaggagaggaaggtaaaggcagtggtgattggtgaataatcactgtcagccaggctaaaagctcagagcatgtacagtcatcagagtcagtcagactgtgatgagtgacatgatccgacgtgaatgcccccacaaaagggcacatcccattccgactgactgattgcaacgccaccactcatcactcgccatctttgacagtctccctttcctggatacatgctcgatgctctcctagtaaacactaaacaccgtgcgtgatgctctgttattgatgccctgcttggcagctaccccaccgccatccctgattctccagccacatgctgccaaaagcaactaaaatttgtctctgacgattcttctgccctcgcggccggtccatccaaagcgactggttttttgtttttttttttaattcagacacgaatatgttctgaacatctatctagctctctctctctgagctagatttatgtggtacgaagaaccctcctgttcagatgggcctatgtatgatacgtgttgctcactcgcttgctcctcctcgcagtctcaatggatggatgaatcagttgtgctgatcaaacagacgaacagtcagatagacaggcaaggcaaccagaccatctatctgacctggacgcgccattaattgcaggttcggtcgcacgggcgtgtaccgaagcttcatgttcagcagcccgactgtgctggtgacgacggcggaggggtgcaagcaggtgctgatggacgacgacgcgttcgtgacggggtggcccaaggcgacggttgcgctggtcgggccgcggtcgttcgtggcgatgccgtacgacgagcaccggcgcatccgcaagctgactgccgcgcccatcaacggcttcgacgcgctgacggggtacctgcccttcatcgaccgcaccgtcacgtcgtcgctgcgcgcgtgggcggaccacggcggcagcgtcgagttcctgacggagctgcggcgcatgacgttcaagatcatcgtgcagatcttcctgggcggcgcggaccaggccaccacgcgcgcgctggagcgcagctacacggagctcaactacggcatgcgcgccatggccatcaacctgcccggcttcgcgtaccgcggggcgctgcgcgcgcgccgccgcctcgtggccgtgctgcagggcgtgctggacgagcgccgcgccgccagggccaagggggtctcgggcggaggagtggacatgatggaccggctgatcgaggcccaggacgagcgcgggcggcacctggacgacgacgagatcatcgacgtgctcgtcatgtacctcaacgccggccacgagtcgtccggccacatcaccatgtgggccaccgtgttcctgcaggagaacccggacatgttcgcgagagcaaaggcaggttctttctttctttctttctttgttgtttaattttatcctgaaagctagctggacaattaagtgcgtgctcttcattcccatgccaccaggcggagcaggaggcgatcatgaggagcatcccgtcgtcgcagcgggggctgacgctcagggacttcaggaagatggagtacctgtcgcaggtctgctccgatccatccattctctgacgatgctagcttgcatcagtgcaaaaacatgctccatggcctgactgactgactgatcccgtgaacgaaggtgatcgacgagacgctgcggctcgtcaacatctccttcgtctccttccgtcaggccaccagagacgtcttcgtgaacggtacgctgcatgctgttgctgtgcggcttcattcattcaggaacctcacctcgctctggaacaggatacctcatccccaaggggtggaaggttcagctctggtaccggagcgtgcacatggacccacaggtgtaccctgaccccaccaagttcgacccgtcgagatgggagggccactcgccgagagccggcacgttcctggcgttcgggctgggcgccaggctctgccccggcaacgacctcgccaagctggagatttccgtcttcctccaccacttcctcctcggctacaagtaactaaataactatctgtgtcgtcgagacgacgattattcctttctttttttttagctggtcgagcagatgaactgttgctgatcgacgggcatgcgtgcgtgcaggctggcgaggacgaaccctaggtgccgggtgaggtacctgccgcacccgaggccggtggacaactgcttggccaagatcaccagagtcggtagctagctgctagattgtgatggaagtacgaatgaaaaaaaagcccagcagaaactctagcggctgggccggctggaaattatgggccgggccattgtggttgccccccctgtctgtgcaaaaataaaacaagcagaaaggaataaaaaaaaggaaaagtattacttcgaacgctgaaattcttggtcgggtcgggcctgttttcggcccatctcaggcagccaggtgcgctcgtgtcgtcccactgtctcgaaatcaaaa

SEQ ID NO: 18 - DNA, 4039 bp, synthetic construct
  source 1..4039: mol_type = other DNA
ccaaaagccggccacttacttgcgtctgcataatactacaaggcatccggcctatatgccgggtgatcgatgatgatgatgatataaaactccgaggaccaaaaaagaaaaaaccgtacgttaccaattaagagcttttgctttcaagaagaatttctctatacaggcgtgcatggtgggtgtttgatttcatttttggaacgtaatggtgtttgatttctagcacagacagagacagagggtgtggcgttacacatctctgcctatatataaacaggaggcgttgcgtgcagagctgccggctggcggttgcctctcctcccctcccctgccctcgcaaacgcacgcaccacagcatcgtccttcgttccattccggcattcccatccatataccccctcgatcggccgggatgctcggcgttggcatggcggccgcggttctcctgggggccgtggccctattgttggcagatgccgcagcgcgtcgagcccattggtggtatcgcgaggccgcggaggctgtgcttgtgggagcggtagccctggtggtggtcgatgccgccgcacgaagggcgcatggatggtatcgggaggccgctcttggagccgcgcggagggcccgccttccccccggcgagatgggttggcctctcgtcggggggatgtgggccttcctgcgcgccttcaagagcggcaaacccgatgccttcattgcgtccttcgtgcggcggtgagtcagtgcgctaatcctcgatctcctccctcctcccctgcacgctcgctccgctctgctcctaccgaggatgggccgggccccacgtgtcattgtcctcgtcgacacctgcacctgcacgcaccgggccccacggccgtgtgagcttgtggcgcatgggtgcaccggagcagcattagctagccagcagcaatggatagctgtcgattttctatcccaggctggttaattaatcctcatccctttgcctgctaaataggagtattacttgtactgaaatcaccaaaatctagaacataaccgttatgacagctgtatatatagaatatgaatagttaaatggctcgtcaagccaaattttatctaatcaacagcaaaataactacatatatacaacaaagtaaccatctaaataactcatcaagctatttgtctctttaaattggctctccctcacaggccaaacttgactagctaaactagatagatagtctattggattgagatgttacacatagaatgtaatctttatagaaaagtaaatagatagtcaaataaagagaaaaaaaatgaagagtactgtgaagatgctctactagtagtaccagaaattaagaagtatatataactcacataggcatgcattgcacttgtagtcgcaagcaattcactgctgttacttttgtaaacgaagcagtcagacaggagagctatcgagtatcaaaagaaggccagaccagactgagacagggtagaacgacggtcgacgacgacgtcccgcgcgcaggagaggaaggtaaaggcagtggtgattggtgaataatcactgtcagccaggctaaaagctcagagcatgtacagtcatcagagtcagtcagactgtgatgagtgacatgatccgacgtgaatgcccccacaaaagggcacatcccattccgactgactgattgcaacgccaccactcatcactcgccatctttgacagtctccctttcctggatacatgctcgatgctctcctagtaaacactaaacaccgtgcgtgatgctctgttattgatgccctgcttggcagctaccccaccgccatccctgattctccagccacatgctgccaaaagcaactaaaatttgtctctgacgattcttctgccctcgcggccggtccatccaaagcgactggttttttgtttttttttttaattcagacacgaatatgttctgaacatctatctagctctctctctctgagctagatttatgtggtacgaagaaccctcctgttcagatgggcctatgtatgatacgtgttgctcactcgcttgctcctcctcgcagtctcaatggatggatgaatcagttgtgctgatcaaacagacgaacagtcagatagacaggcaaggcaaccagaccatctatctgacctggacgcgccattaattgcaggttcggccgcaccggggtctaccgctccttcatgttcagcagcccgactgtgctggtgacgacggcggaggggtgcaagcaggtgctgatggacgacgacgcgttcgtgacggggtggcccaaggcgacggttgcgctggtcgggccgcggtcgttcgtggcgatgccgtacgacgagcaccggcgcatccgcaagctgactgccgcgcccatcaacggcttcgacgcgctgacggggtacctgcccttcatcgaccgcaccgtcacgtcgtcgctgcgcgcgtgggcggaccacggcggcagcgtcgagttcctgacggagctgcggcgcatgacgttcaagatcatcgtgcagatcttcctgggcggcgcggaccaggccaccacgcgcgcgctggagcgcagctacacggagctcaactacggcatgcgcgccatggccatcaacctgcccggcttcgcgtaccgcggggcgctgcgcgcgcgccgccgcctcgtggccgtgctgcagggcgtgctggacgagcgccgcgccgccagggccaagggggtctcgggcggaggagtggacatgatggaccggctgatcgaggcccaggacgagcgcgggcggcacctggacgacgacgagatcatcgacgtgctcgtcatgtacctcaacgccggccacgagtcgtccggccacatcaccatgtgggccaccgtgttcctgcaggagaacccggacatgttcgcgagagcaaaggcaggttctttctttctttctttctttgttgtttaattttatcctgaaagctagctggacaattaagtgcgtgctcttcattcccatgccaccaggcggagcaggaggcgatcatgaggagcatcccgtcgtcgcagcgggggctgacgctcagggacttcaggaagatggagtacctgtcgcaggtctgctccgatccatccattctctgacgatgctagcttgcatcagtgcaaaaacatgctccatggcctgactgactgactgatcccgtgaacgaaggtgatcgacgagacgctgcggctcgtcaacatctccttcgtctccttccgtcaggccaccagagacgtcttcgtgaacggtacgctgcatgctgttgctgtgcggcttcattcattcaggaacctcacctcgctctggaacaggatacctcatccccaaggggtggaaggttcagctctggtaccggagcgtgcacatggacccacaggtgtaccctgaccccaccaagttcgacccgtcgagatgggagggccactcgccgagagccggcacgttcctggcgttcgggctgggcgccaggctctgccccggcaacgacctcgccaagctggagatttccgtcttcctccaccacttcctcctcggctacaagtaactaaataactatctgtgtcgtcgagacgacgattattcctttctttttttttagctggtcgagcagatgaactgttgctgatcgacgggcatgcgtgcgtgcaggctggcgaggacgaaccctaggtgccgggtgaggtacctgccgcacccgaggccggtggacaactgcttggccaagatcaccagagtcggtagctagctgctagattgtgatggaagtacgaatgaaaaaaaagcccagcagaaactctagcggctgggccggctggaaattatgggccgggccattgtggttgccccccctgtctgtgcaaaaataaaacaagcagaaaggaataaaaaaaaggaaaagtattacttcgaacgctgaaattcttggtcgggtcgggcctgttttcggcccatctcaggcagccaggtgcgctcgtgtcgtcccactgtctcgaaatcaaaa

SEQ ID NO: 19 - DNA, 2384 bp, Zea mays
  source 1..2384: mol_type = other DNA
ccacaaaaaaaaacacacacacaggggagggagacgagacgagatccaaaattccctggcggtgggctgtgggctgtggctggtaataaagctcgcgccacgggagccacgcacgcggacgccggcatattcgccaaacgcatcgcatcggcctcagccaccggcgcggctgccctcctcttctccgtcgatccggagaggactttctctcgcctgctcgtctctcccgcccccgccggccggccggtcctcttcccggcccactccattccgatcgaccatggcggctaccgcccccaacggcaatcccactgtcaggtacggctcccgcctcgttccatcatgcgatgagagttaactacccccccccccccccccccccccccccccgtcgcgtgaacagagcgccatccaatgacgccacttcgttgcggttttagatttaccacccactcgttccgagcctgtgcgtggtcaatatgtgtgtgagatgaattacatactgtttctgctcttcttcttctgaaaccgtcgagttgcgtgactaccatcgagcgatgcacagcctgagatttatcgcgcgcgcacaaacacgcatactcgttccaagttcctgatcacctgatgattcaaatgatgggcaattgccaattgggcatgatctgcagctccctcgcggcaaccgttccggtccaggcggttctgttcgacatcgacgggaccctgtgcgactcggatcccctccaccacgtcgctttccaagagatgcttctcgaggtacggtggaacgtcgcttttccctgtttcacttttctttttttaaaccaacgagctgcctgcgatgtgatgtggtttgatttacgaaaccaaaatggctggcatggcatacgcagatcgggtacaacaatggcgtgccgatagacgaggagttcttcatcaagaacatcgctgggaggagtgacgtcgaagctgcccagaacctgttcccggactgggagcttgagaaggggctcaaattcctagaggacaaggaagccaaatacaggaggtacgtacaaatgatagctgtggttttcctcagtattagatcaactgaaagattcagagagtaagcgtgttgtccattgttacaagtttacaacagtaaacccctttgcgtaccgtagcacatacataaataacatccatttcagcgctatcacgcaaaaaaaaaacaccaactttttgctactcagagagatcgattactaggttgtggagagttctgtcatttcctacagcgcccattcgctgctgattctgcggaatgtgatcctttcataatcattacaatacattgcattgcattacagaactgataggttcgttatgacaacgtgcatgagtgactgaatgtttctagttgttggttctttttattcagtctggcaaaagagcgcttggtgcctgtgaaaggcctcgcgaaggtggtccagtgggtgaaagaccacgggtacaagcgcgcggcggtgaccaacgcccccaggatcaacgcggagctgatgatctcgctcctgggcctctcggacttcttccaggccgtcatcgtcggaggcgagtgcgagcagccaaagcccgcgccgtacccttacctcagggccctcaaggagctccaggtgtccgcagagcacagcttcgtcttcgaggtaaaatcaaacgaccttgtgactgggattctgtgatctgatggttgtattacaggagtagtcactgataaaagatgagatttctttttgggttggtgtaggattctcctgctggtatccgggctggcgtcgcggcggggatgcctgtcgtcggtgtggcgacgaggaacccggagaagtccttggtggaagcaggagccgcgttgctcgtcaaggactacgaagatcctaagctctgggcagcgcttgaggagatggacggagaggaagctaagctgaagaaggccagcgaatgatctgatatgacgatgagccattgattagatttattgcagattaaatctgcctccagcatgattctgttcctagttatgcgctgcggagagagctcacctcatcagggtatttatgagattctagcttcgtatattgtttttcttcttcttgattgaactgagatcaaataagacaaagctgctgtgccttgttgtgtgccatctgtgtatgaacttttcggttcagcctccagcagtctttaatctgaaagaaagacgaggtaggaattctctccctcagggaagatgtgtgaagaatgcaccgtgatgcttgttgcttgtcgaatccatggacgtcatcctttttgagcttcagctctgctacgcaaaccgtgttgaaagtggtttcgggggctatgcagca

SEQ ID NO: 20 - DNA, 2384 bp, synthetic construct
  source 1..2384: mol_type = other DNA
ccacaaaaaaaaacacacacacaggggagggagacgagacgagatccaaaattccctggcggtgggctgtgggctgtggctggtaataaagctcgcgccacgggagccacgcacgcggacgccggcatattcgccaaacgcatcgcatcggcctcagccaccggcgcggctgccctcctcttctccgtcgatccggagaggactttctctcgcctgctcgtctctcccgcccccgccggccggccggtcctcttcccggcccactccattccgatcgaccatggccgccacggctcctaacggtaaccctaccgttaggtacggctcccgcctcgttccatcatgcgatgagagttaactacccccccccccccccccccccccccccccgtcgcgtgaacagagcgccatccaatgacgccacttcgttgcggttttagatttaccacccactcgttccgagcctgtgcgtggtcaatatgtgtgtgagatgaattacatactgtttctgctcttcttcttctgaaaccgtcgagttgcgtgactaccatcgagcgatgcacagcctgagatttatcgcgcgcgcacaaacacgcatactcgttccaagttcctgatcacctgatgattcaaatgatgggcaattgccaattgggcatgatctgcagtagcctggcggccaccgtgccggtccaagcggtgctgttcgacatcgacggcacgctctgtgattctgacccactgcaccacgtagccttccaggagatgctcctggaagtacggtggaacgtcgcttttccctgtttcacttttctttttttaaaccaacgagctgcctgcgatgtgatgtggtttgatttacgaaaccaaaatggctggcatggcatacgcagataggctacaataacggtgttcctattgatgaagaattcttcatcaagaacatcgccggccgctccgacgtcgaggctgcgcaaaatttgtttcctgactgggaacttgagaaaggtctcaagttcctagaggacaaggaagccaaatacaggaggtacgtacaaatgatagctgtggttttcctcagtattagatcaactgaaagattcagagagtaagcgtgttgtccattgttacaagtttacaacagtaaacccctttgcgtaccgtagcacatacataaataacatccatttcagcgctatcacgcaaaaaaaaaacaccaactttttgctactcagagagatcgattactaggttgtggagagttctgtcatttcctacagcgcccattcgctgctgattctgcggaatgtgatcctttcataatcattacaatacattgcattgcattacagaactgataggttcgttatgacaacgtgcatgagtgactgaatgtttctagttgttggttctttttattcagtctggcaaaagagcgcttggtgcctgtgaaaggcctcgcgaaggtggtccagtgggtgaaagaccacgggtacaagcgcgcggcggtgaccaacgcccccaggatcaacgcggagctgatgatctcgctcctgggcctctcggacttcttccaggccgtcatcgtcggaggcgagtgcgagcagccaaagcccgcgccgtacccttacctcagggccctcaaggagctccaggtgtccgcagagcacagcttcgtcttcgaggtaaaatcaaacgaccttgtgactgggattctgtgatctgatggttgtattacaggagtagtcactgataaaagatgagatttctttttgggttggtgtaggattctcctgctggtatccgggctggcgtcgcggcggggatgcctgtcgtcggtgtggcgacgaggaacccggagaagtccttggtggaagcaggagccgcgttgctcgtcaaggactacgaagatcctaagctctgggcagcgcttgaggagatggacggagaggaagctaagctgaagaaggccagcgaatgatctgatatgacgatgagccattgattagatttattgcagattaaatctgcctccagcatgattctgttcctagttatgcgctgcggagagagctcacctcatcagggtatttatgagattctagcttcgtatattgtttttcttcttcttgattgaactgagatcaaataagacaaagctgctgtgccttgttgtgtgccatctgtgtatgaacttttcggttcagcctccagcagtctttaatctgaaagaaagacgaggtaggaattctctccctcagggaagatgtgtgaagaatgcaccgtgatgcttgttgcttgtcgaatccatggacgtcatcctttttgagcttcagctctgctacgcaaaccgtgttgaaagtggtttcgggggctatgcagca

SEQ ID NO: 21 - DNA, 1495 bp, Zea mays
  source 1..1495: mol_type = other DNA
tggaacatgagaatgtgagagactaccaccaaccgcgccaactgaaccacggaatcctcaccaactctccctgcctcttcagtcttctccttataaaatgtgcaggcagcatcagcactcgagcagtgaccacaagaccaaaagaagcctcagttttggcatccagcagcaactggccatggaaaggtaatcatccagagtattttctgcatgatttcactcagttctattcccccattcccagagagacagaaactgtcaagatttcgtttcaatccgaccaaaactgtcaagatttcatttcaatccgagcaagttctccgcaaccatggccgtcccagtttaaagttttatcttctttccctgtgtgtgtgtgtgtggttgctttgcagcagtgggatcggcagagtggccccactggaggccgtcctcttcgacatcgacggcaccatggccatctccgacccgttccaccaccgggccacctcggagatgctcctcaaggtgggctacaacaacggcgtgcccatcacgcccgagttcggcatggcgaacatggccggccggagcaacgagcagatcggccgcttcctgttcccggactgggaccagcgccgcctcgacgccttcttcgccgagaaggaggcgctcttcgccaggtacgccgccgaggggctccgggagatcgccggcctcacgccgctctgccgctgggccgccggccgcgggctcaggcgcgccgccgtcaccaacgcacccagggccaacgccgagctcatgatctccatcctcggcctctccgactttttttccctcgtcgtcaccgccgaggagtgcggccgatccaagccctaccctgacccgtacctcagggcgctcgacctgctcggcgtctcgccggaccacgcgctcgtgttcgaggactccaccactggcgtgcaggccggcattgcggcagggatgccggtggttgccatcgccgaggaaagcagagaggacaagcttctcgccgtcggcgcaacgctcgtcatcagggactacgaagaccccaagctatgggcggctttggacaaactggacaccaccaggcctcaagctgctgctgctgagaccaatggaaaactcacacaactgtagctgaggaacacaagtgctagtagccaaaaacaatttcttgcaatattccacttccacgcatgattcaataacaactcattcgtgtatgtatgatgtatcttgatgagatgcatacaaaattgcctgtaggccagtctgttacagacaaatgaaaatggaaacaaaactgatcgcgataacatgcagatttctatttgtatctaaactactctaccacaggctctcacatgagattataatgtgcttcagaaatgggtaaaagctggtgacggtgcacactccttgcaaacatggtacagacaccacatttttttcacaaggttgtcctcatatacacactaacatgtggacagctggt

SEQ ID NO: 22 - DNA, 1495 bp, synthetic construct
  source 1..1495: mol_type = other DNA
tggaacatgagaatgtgagagactaccaccaaccgcgccaactgaaccacggaatcctcaccaactctccctgcctcttcagtcttctccttataaaatgtgccggcagcatcagcactcgagctcggatcacaagaccaagaggagcttgtccttcgggattcagcagcaattggccatggagtcgtaatcatccagagtattttctgcatgatttcactcagttctattcccccattcccagagagacagaaactgtcaagatttcgtttcaatccgaccaaaactgtcaagatttcatttcaatccgagcaagttctccgcaaccatggccgtcccagtttaaagttttatcttctttccctgtgtgtgtgtgtgtggttgctttgcagctcgggcatcggtcgggtggcacccctggaggccgtgctctttgatattgacgggactatggctatctccgacccattccaccaccgtgccaccagcgagatgttgctgaaggtcgggtataataacggggtgccgattactcccgagttcgggatggcgaacatggccggtcgctctaatgagcagatcggccggttcctattcccggactgggatcagcggcgcctcgacgccttcttcgctgagaaggaggcgttattcgcccgctacgcagcggaggggctccgcgaaatcgcggggctcactccgctgtgccgctgggccgccggccgcgggctcaggcgcgccgccgtcaccaacgcacccagggccaacgccgagctcatgatctccatcctcggcctctccgactttttttccctcgtcgtcaccgccgaggagtgcggccgatccaagccctaccctgacccgtacctcagggcgctcgacctgctcggcgtctcgccggaccacgcgctcgtgttcgaggactccaccactggcgtgcaggccggcattgcggcagggatgccggtggttgccatcgccgaggaaagcagagaggacaagcttctcgccgtcggcgcaacgctcgtcatcagggactacgaagaccccaagctatgggcggctttggacaaactggacaccaccaggcctcaagctgctgctgctgagaccaatggaaaactcacacaactgtagctgaggaacacaagtgctagtagccaaaaacaatttcttgcaatattccacttccacgcatgattcaataacaactcattcgtgtatgtatgatgtatcttgatgagatgcatacaaaattgcctgtaggccagtctgttacagacaaatgaaaatggaaacaaaactgatcgcgataacatgcagatttctatttgtatctaaactactctaccacaggctctcacatgagattataatgtgcttcagaaatgggtaaaagctggtgacggtgcacactccttgcaaacatggtacagacaccacatttttttcacaaggttgtcctcatatacacactaacatgtggacagctggt

SEQ ID NO: 23 - DNA, 4512 bp, Zea mays
  source 1..4512: mol_type = other DNA
agttctttaccactaccggcccagacacctaaacgatgacacagcgtgcacccaccaccaccagcgccgagcgccggccgcgataccgtcgtgtccccctccccaccgcattagcgccggccgacaaaattactaggtgcttaatttaactactagtggccctaggcgtgaaacttcaccagctccgccactgcccatggccaacggccgtccggaccctcgctgcgtcgtcctcaccgccgacactgcccgatccctcgtagattccgtcgacgccttcctcttcgactgcgatggtatctgattgttcacctttctccattttgtttgtctgtttttttttcttcgcttcagattttgacccctgcttcgctctgaccggtaattctaattgcttctctgaatttgtctaggggtcatttggaagggagataagctcattgaaggggtcccagagacgatggagctactgaggaaactggtatgggaacatccctttattactatttaagtattgaattcgtttcctcattttcaatagttcaactgtcaggcaaataaatgccaaatgatcaaaacaagtacataattaagggctagcttgttttgtcgtgccttaaaaaaaaaaactggttctagattgattggggctttgaagaatctggttctccaaagttgcatctatttaagctaaaatagagacaggcagtaagtgcaaccacaccacaccaggcaccagataccgtaatgaaatgaaaatcattgacactactactactacattgctttaaagttattctggttttgttcatctacataactcctgtctgaaacaaggataataggcctatatcttttttctatgtgaattctagcatgatgaactcacctcattaaccaagatgtgatctttgttgcgccattatttaagacattgctctagtactcggtggcttattggtttatagaaaaaggtatatacagattatgaaatttattaattgagtaatagtgcatgtattggcttggtgcagggaaagaaattagtttttgtaacaaacaactctagaaagtcgagaaggcaatactcaaagaaattcagatcacttggacttgaagttactgaggtatgttttggtctgagcggagactcttccagtaataaatttgttttgtattgtaatcatttgttactttgttgtaggaagagatttttacatcatcgtttgcggcagccatgttcttgaagttaaataattttcctccagaaaagaaggtatcataaattattgtgatgttctttccttttgttttatgtgaatgacttatgatttcttcggcacatgatacttactgaaattatcaccatgaggaaacaaattaagaaagtggcacatatgtacgcggggaatttgatagcacaacagcctacacatttacagtaagattggtcacgccgaactaatgagtaagggtttgccataagactggaacagctatgtgggaactatgtggttgcgattttcactagaagaccaatcaaaaggcacttatatgagccaatcaaccttgcttcacataacaggagtgttgtagatagtttgggtccgctatttcctgtatacctcactttatttatcttgatttttcttgtgtctggtcttacttgaactgcttatttttctttaggtttatgttgttggtgaagatggaatcttggaagagctcaagttagctggttttgaatgttttggtggtccggtaagacttgcttattctcctcccccaacacacacacacctttctctctcctgattactaatcatttagtttataacaactgttttctgctgaggggatggaaaccatgttaagtgttcaagattttcttgaatttctatctgttatcctttttcttagagttcctttactataatgtgaagatgctatgtggtatactgaatactggtttgatatagttctaacggtgcatccgtgctaatggatcatttttttcatttgcgaacttctctatacattggcatattctaggttctataattttcataatcatagttgacaataaaatataaacatagtggatcattgtatgggatattacctggttgatcgggagtgcctacttcaaggtattacatgttacttcgatgtagtagtcactgtgtgtgtgtgtgtgtgtgtgtgtgtgtgtgtgtgtgtgtgtgtgtgtgtgtgtgtgtgtgtgtgtgtgtgtgtgtgtgttgtgctatatgaaataaaataggccagaaacatgaagtgcattataaaaacatgcctaagtgtcctgagagagccatagcatacggacagtcaaaagattttttttgtatacttgatgagggcttgaaaacataaagatttttcagataagttatccctgttttaccttccatttcaactaatgattttttctgcaggaggatggcaagaaaaacataaagttggaggcggatttctactttgaacatgacaaaagtgtaatgtcaaatctgtttttctgatttttgtatcatctttcacataaaatacaaaagctactcagctgtgatctaaatgaaatttcttttccaggttggagctgtcattgttggacttgatcagtacttcaattattataaaatgcagttattgctctaaccttgctgtctatagccttacctttggcgttgctctccatgctgatcaatcattgcttcacttttgtcatacgtgtattttcttccgtttcaatgcaagtttcatctcgcttctggaaaattgaacgagataaaactacattaaggtccatttgattgatctttcttttcaccatatttttccggtatgttggatgctccacccttggcccttgcctaggcagttgagcttcctttgtcacatgatttagaacatggtatattacatatgatatagaccgagccctcccagatatgcttgttgtgcatttcgttttcatttcctttggtaactcgtgtctcctttaaaccagcggagtaaagtttcattgcttgctcagtgttggtacctctatgttgttcgccgttacttaaaacgtttgttgaaagagtacccgttatttaattcagacctaaaattcagaagcttctaatttgagataatccagccaagcaattataatctgccttatttttatgctttataaagacatatcacagattcacagttccagttttaaacttttgtcggcaaggaccacgttaccacaataaaagtactccgtctgttttttgtagtaaatggaaaataaactaattagcatgtactagagtaatatgttcaaaaaacggagtaagaatttatttgagcatcatttttcaacattagttgtgaccgcagcccatgattctgtttaccttctagatcattagtttgacaatgatcgagtcatagcgatgtgcttgcaggtatgcaagaacatgtattagtgagaaccctgactgccttttcattgcgaccaaccgtgatccaaccgggcatatgacatctgcccaagaatggccaggtattgttaatgtaaaatttattgagaatttttttttttaagtttcgatctgtagcacagtataattggcaagtggttacctcaaacttaacaggagctggagctatggttgctgcagtaagctgctcagtacagaaagaacccattgttgttggaaaaccttcaggctttttgatggacttcctcttgaaaaggtttgcgagttgtattatgagagaaaaaatatatcatttattcataagttcaggggattgcatgctttgttgatttttcaatggcacagtatgagatataaggtttccctgctgcagcttcaatctggagacgtcaaggatgtgcatggttggtgatagactggacacggacatactatttggccagaacaccggctgcaagaccctccttgttttgtctggtcagaggctcctgctctttcttgtatcccgaaccgctttctttctttctttctttctttctcgtgttcaatgatgatgatgatgtgtctcacgtgcgtgggattgcaattgcaggttgcactgccttaccagaagtgcaggatgctcgtaacaatatccatccagatctctacacaaacagcgtgtatgatctagttgggttactgcagaagtaattctccttggccgcgttgcagcgcaatctcagcacgctttggatagcgcctatcagcaatttattacgtggattacttgcatgaagccatgtgttcttgaaacaacaattttgaactttctttggggtataacctgtttgacagtaacaaaatctttcaattggatcctacaaattagaccaatttgacaaccttgtaagcgtggctgtaaagctttaaatctggcaacttgattcccttgctggaaaatatattggttggagtatggaattgtacagaatcgaattgtaatgaagcaacgtgttgtactttgttg

SEQ ID NO: 24 - DNA, 4512 bp, synthetic construct
  source 1..4512: mol_type = other DNA
agttctttaccactaccggcccagacacctaaacgatgacacagcgtgcacccaccaccaccagcgccgagcgccggccgcgataccgtcgtgtccccctccccaccgcattagcgccggccgacaaaattactaggtgcttaatttaactactagtggccctaggcgtgaaacttcaccagctccgccactgcccatggccaacggccgtccggaccctcgctgcgtcgtcctcaccgccgacactgcccgatccctcgtagattccgtcgacgccttcctcttcgactgcgatggtatctgattgttcacctttctccattttgtttgtctgtttttttttcttcgcttcagattttgacccctgcttcgctctgaccggtaattctaattgcttctctgaatttgtctaggggtcatttggaagggagataagctcattgaaggggtcccagagacgatggagctactgaggaaactggtatgggaacatccctttattactatttaagtattgaattcgtttcctcattttcaatagttcaactgtcaggcaaataaatgccaaatgatcaaaacaagtacataattaagggctagcttgttttgtcgtgccttaaaaaaaaaaactggttctagattgattggggctttgaagaatctggttctccaaagttgcatctatttaagctaaaatagagacaggcagtaagtgcaaccacaccacaccaggcaccagataccgtaatgaaatgaaaatcattgacactactactactacattgctttaaagttattctggttttgttcatctacataactcctgtctgaaacaaggataataggcctatatcttttttctatgtgaattctagcatgatgaactcacctcattaaccaagatgtgatctttgttgcgccattatttaagacattgctctagtactcggtggcttattggtttatagaaaaaggtatatacagattatgaaatttattaattgagtaatagtgcatgtattggcttggtgcagggaaagaaattagtttttgtaacaaacaactctagaaagtcgagaaggcaatactcaaagaaattcagatcacttggacttgaagttactgaggtatgttttggtctgagcggagactcttccagtaataaatttgttttgtattgtaatcatttgttactttgttgtaggaagagatttttacatcatcgtttgcggcagccatgttcttgaagttaaataattttcctccagaaaagaaggtatcataaattattgtgatgttctttccttttgttttatgtgaatgacttatgatttcttcggcacatgatacttactgaaattatcaccatgaggaaacaaattaagaaagtggcacatatgtacgcggggaatttgatagcacaacagcctacacatttacagtaagattggtcacgccgaactaatgagtaagggtttgccataagactggaacagctatgtgggaactatgtggttgcgattttcactagaagaccaatcaaaaggcacttatatgagccaatcaaccttgcttcacataacaggagtgttgtagatagtttgggtccgctatttcctgtatacctcactttatttatcttgatttttcttgtgtctggtcttacttgaactgcttatttttctttaggtttatgttgttggtgaagatggaatcttggaagaactaaaactagcaggattcgaatgcttcggaggaccagtaagacttgcttattctcctcccccaacacacacacacctttctctctcctgattactaatcatttagtttataacaactgttttctgctgaggggatggaaaccatgttaagtgttcaagattttcttgaatttctatctgttatcctttttcttagagttcctttactataatgtgaagatgctatgtggtatactgaatactggtttgatatagttctaacggtgcatccgtgctaatggatcatttttttcatttgcgaacttctctatacattggcatattctaggttctataattttcataatcatagttgacaataaaatataaacatagtggatcattgtatgggatattacctggttgatcgggagtgcctacttcaaggtattacatgttacttcgatgtagtagtcactgtgtgtgtgtgtgtgtgtgtgtgtgtgtgtgtgtgtgtgtgtgtgtgtgtgtgtgtgtgtgtgtgtgtgtgtgtgtgtgttgtgctatatgaaataaaataggccagaaacatgaagtgcattataaaaacatgcctaagtgtcctgagagagccatagcatacggacagtcaaaagattttttttgtatacttgatgagggcttgaaaacataaagatttttcagataagttatccctgttttaccttccatttcaactaatgattttttctgcaggaagacggaaaaaaaaacataaaactagaagcagacttctacttcgaacacgacaaatcagtaatgtcaaatctgtttttctgatttttgtatcatctttcacataaaatacaaaagctactcagctgtgatctaaatgaaatttcttttccaggtaggagcagtaatagtaggactagaccaatacttcaactactacaaaatgcagttattgctctaaccttgctgtctatagccttacctttggcgttgctctccatgctgatcaatcattgcttcacttttgtcatacgtgtattttcttccgtttcaatgcaagtttcatctcgcttctggaaaattgaacgagataaaactacattaaggtccatttgattgatctttcttttcaccatatttttccggtatgttggatgctccacccttggcccttgcctaggcagttgagcttcctttgtcacatgatttagaacatggtatattacatatgatatagaccgagccctcccagatatgcttgttgtgcatttcgttttcatttcctttggtaactcgtgtctcctttaaaccagcggagtaaagtttcattgcttgctcagtgttggtacctctatgttgttcgccgttacttaaaacgtttgttgaaagagtacccgttatttaattcagacctaaaattcagaagcttctaatttgagataatccagccaagcaattataatctgccttatttttatgctttataaagacatatcacagattcacagttccagttttaaacttttgtcggcaaggaccacgttaccacaataaaagtactccgtctgttttttgtagtaaatggaaaataaactaattagcatgtactagagtaatatgttcaaaaaacggagtaagaatttatttgagcatcatttttcaacattagttgtgaccgcagcccatgattctgtttaccttctagatcattagtttgacaatgatcgagtcatagcgatgtgcttgcagatacgcaagaacatgcatatcagaaaacccagactgcctattcatagcaacaaacagagacccaacaggacacatgacatcagcacaagaatggccaggtattgttaatgtaaaatttattgagaatttttttttttaagtttcgatctgtagcacagtataattggcaagtggttacctcaaacttaacaggagcaggagcaatggtagcagcagtatcatgctcagtacaaaaagaaccaatagtagtaggaaaaccatcaggattcctaatggacttcctactaaaatcgtttgcgagttgtattatgagagaaaaaatatatcatttattcataagttcaggggattgcatgctttgttgatttttcaatggcacagtatgagatataaggtttccctgctgcagattcaacctagaaacatcaagaatgtgcatggtaggagacagactagacacagacatactattcggacaaaacacaggatgcaaaacactactagtactatcaggtcagaggctcctgctctttcttgtatcccgaaccgctttctttctttctttctttctttctcgtgttcaatgatgatgatgatgtgtctcacgtgcgtgggattgcaattgcaggatgcacagcactaccagaagtacaagacgcaagaaacaacatacacccagacctatacacaaactcagtatacgacctagtaggactactacaaaaataattctccttggccgcgttgcagcgcaatctcagcacgctttggatagcgcctatcagcaatttattacgtggattacttgcatgaagccatgtgttcttgaaacaacaattttgaactttctttggggtataacctgtttgacagtaacaaaatctttcaattggatcctacaaattagaccaatttgacaaccttgtaagcgtggctgtaaagctttaaatctggcaacttgattcccttgctggaaaatatattggttggagtatggaattgtacagaatcgaattgtaatgaagcaacgtgttgtactttgttg

SEQ ID NO: 25 - DNA, 1583 bp, Zea mays
  source 1..1583: mol_type = other DNA
ccaggggccaacacagcaccgacacctaaaaaaaatggtcgtgcacaggagcgttgcacttgcagctgatctcatctgatccgatcaatcaacgtgacatgatctgcagacggtcgaatgatgcttcagtaaaatatttttataagtttacggtcggtccctcaaatcatcatcatcaccaatattataattgttaccaacagctcgcctgcccaccaaatcggcaaatcctggcgtacaggaggacgacccaacccaagcggcacacaagatccaagaggatgccgccgccgccgccgctcgtgctgctggcgaagccgctgttcccgaacttcgcggcggcgctggagggccggtaccgcctcgtcctggccggggacgccgacgcggccacggcggcggaggcccgggtcctgctcgtgccggggctcgtggccgtgacagccgagctcgtcgaccggctcccggcgctggagctcgtggccgcgacctccgtcgggctggaccacgtggacctccgtgcctgccgccgccgcgggctcgccgtcaccaacgccggcgcggccttctccgtcgactccgccgactacgccgtcggcctcgtcgtcgccgtgctgcgcagggtcgccgccgccgaggcgcatctccggcgcggcgggtgggctaccgatggcgagtacccgctcaccaccaaggtatagcagcatactgagaattgccacccaaaaaaaacaaacaaacagaagaacagaattcgacatgtctcggactctcggttgaattaatttgcaggtgagcggcaagcgggtggggatcgtggggctcggcagcatcggctcgctggtggcgcggcgcctggccgccatggggtgccgcgtcgcctaccactcccgcgcgccgaagccgtcgtgctcgtgcccctacgccttcttccccacggcgcgcgccctggccttggccagcgacgtgctggtgctgtcgtgcgcgctcacggaggagacgcggcgcgtggtgggccgcgaggtgctggaggcgctgggacagggcggcgtgctggtcaacgtcggccgcggcggcctggtcgacgagccggagctggtgcggtgcctgcgggagggcgtcatcggcggcgcggggctggacgtgttcgaggacgagcccgacgtgcccgcggagctcctcgccatggacaacgtcgtgctgtcgccccacagggccgtgctcacgccggagtccatgcgcgggttgctggatgtcgtcgcgggaaacctcgacgccttcttcgccggcaggccgctgctcagccccgtgtcgctgtgaattctgaaccacatgtaggatagatacgtcaagtcgtcaagttccattccatccaagaagcatcaaccagacaaccagtgtgtgagactcaccacgaacaatacggaagaacactcagaattggtagggtggttcgggtttcttactttctgttccaagttcatcatccaaaagccaaatttcaaaatgacaacagttcgtcttttatacttgctactggaaaaccaaaacgaattggaatatattacagccagttggctcca

SEQ ID NO: 26 - DNA, 1583 bp, synthetic construct
  source 1..1583: mol_type = other DNA
ccaggggccaacacagcaccgacacctaaaaaaaatggtcgtgcacaggagcgttgcacttgcagctgatctcatctgatccgatcaatcaacgtgacatgatctgcagacggtcgaatgatgcttcagtaaaatatttttataagtttacggtcggtccctcaaatcatcatcatcaccaatattataattgttaccaacagctcgcctgcccaccaaatcggcaaatcctggcgtacaggaggacgacccaacccaagcggcacacaagatccaagaggatgccaccgccaccacctctggtcttgttggccaagcccctcttccccaatttcgccgccgcgctggagggccgctaccggctcgtgctggcaggcgacgccgacgccgccaccgccgccgaggcccgggtcctactggtcccgggcctggtggcggttacggcggagttggtggatcgcctgccagcactagagctggtagcggccaccagcgtggggctcgaccacgtagatctccgcgcttgtcgacggagagggctggccgtgactaatgcaggggcagccttctccgtcgacagcgcggactacgcggtcgggctggtagtcgccgtccttcgccgagtcgccgcggcggaggcacacctccgccgcggcggctgggcgacggacggcgagtacccgttgacgacgaaggtatagcagcatactgagaattgccacccaaaaaaaacaaacaaacagaagaacagaattcgacatgtctcggactctcggttgaattaatttgcaggtgtcgggcaagagagttgggattgtggggctcgggagtataggcagcctggtagcccgtcgcctagcggcgatggggtgccgcgtcgcctaccactcccgcgcgccgaagccgtcgtgctcgtgcccctacgccttcttccccacggcgcgcgccctggccttggccagcgacgtgctggtgctgtcgtgcgcgctcacggaggagacgcggcgcgtggtgggccgcgaggtgctggaggcgctgggacagggcggcgtgctggtcaacgtcggccgcggcggcctggtcgacgagccggagctggtgcggtgcctgcgggagggcgtcatcggcggcgcggggctggacgtgttcgaggacgagcccgacgtgcccgcggagctcctcgccatggacaacgtcgtgctgtcgccccacagggccgtgctcacgccggagtccatgcgcgggttgctggatgtcgtcgcgggaaacctcgacgccttcttcgccggcaggccgctgctcagccccgtgtcgctgtgaattctgaaccacatgtaggatagatacgtcaagtcgtcaagttccattccatccaagaagcatcaaccagacaaccagtgtgtgagactcaccacgaacaatacggaagaacactcagaattggtagggtggttcgggtttcttactttctgttccaagttcatcatccaaaagccaaatttcaaaatgacaacagttcgtcttttatacttgctactggaaaaccaaaacgaattggaatatattacagccagttggctcca

SEQ ID NO: 27 - protein, 256 aa, Arabidopsis thaliana
MGIQKPAWLDALYAEKFFVGCPYHETAKKNERNVCCLDCCTSLCPHCVPSHRFHRLLQVRRYVYHDVVRLEDLQKLIDCSNVQAYTINSAKVVFIKKRPQNRQFKGAGNYCTSCDRSLQEPYIHCSLGCKVDFVMKRYRDITPFLKPCHTLTLGPDYIIPQDLLTDDEVAAYETPRSTVVDGDESMSWSSASSDNNNAGAAAAYAATTTHVVRKKRTGFCLCAKSANSYKEVSEDPDDISACINRRKGVPQRSPLC

SEQ ID NO: 28 - protein, 236 aa, Zea mays
MIMAMWKPGWLEALDTQKFFVACSFHEHAKKNEKNICCLDCCTSICPHCVAAHRAHRLLQVRRYVYHDVVRLEDLEKLIDCSSVQSYTINSSKVVFLKKRPQNRQFKGSGNICTSCDRSLQEPYFHCSLDCKVEYILRQKKKLSAYLRPCKTLQLGPDFFIPHDADDDTTHSTLVDVDEPMGSSDSENLSVPCTNFVRKKRSGPYICARSANRVSEEDMATNMSRRKGVPQRSPLC

SEQ ID NO: 29 - RNA, 711 nt, Zea mays
  source 1..711: mol_type = other RNA
atgatcatggcaatgtggaagccaggatggctagaggcccttgacacacagaagttcttcgtagcatgctctttccatgagcatgccaagaagaacgagaagaacatctgttgccttgactgctgcactagcatctgcccacactgtgtggcagcacaccgtgcacacaggctcctgcaggtgcggcgatacgtctaccatgacgttgtccggctggaggacctggagaagcttattgattgctctagtgttcagtcttatactattaacagctctaaggttgttttcctgaagaagagaccacagaataggcaattcaagggttcagggaatatctgcacctcctgcgacaggagccttcaagaaccgtatttccactgctctctggattgcaaggtagagtatatactacgacagaagaaaaaattgtcagcatatttgcgcccatgcaagaccttgcagcttggccctgatttcttcattcctcatgatgctgatgacgacacaactcactcaacccttgttgatgttgatgagcccatgggatcatcggactcggagaatttgagtgtgccgtgcacaaattttgttcggaaaaaacggagtggaccatatatttgtgcacggtctgcaaacagagtgtctgaagaagacatggccacaaatatgagcagaaggaaaggggttcctcagagatcgcctttgtgctaa

SEQ ID NO: 30 - RNA, 711 nt, synthetic construct
  source 1..711: mol_type = other RNA
atgatcatggccatgtggaagccgggctggctggaggctctggacacacaaaagttcttcgtggcgtgctcgttccacgagcacgccaagaagaacgagaagaacatatgttgcctagattgctgcacctcgatctgcccgcattgtgtggccgctcatcgggcgcaccgcctcctccaggtgcgccgctacgtgtaccacgatgtggtgcggctggaggacctggagaagcttattgattgctctagtgttcagtcttatactattaacagctctaaggttgttttcctgaagaagagaccacagaataggcaattcaagggttcagggaatatctgcacctcctgcgacaggagccttcaagaaccgtatttccactgctctctggattgcaaggtagagtatatactacgacagaagaaaaaattgtcagcatatttgcgcccatgcaagaccttgcagcttggccctgatttcttcattcctcatgatgctgatgacgacacaactcactcaacccttgttgatgttgatgagcccatgggatcatcggactcggagaatttgagtgtgccgtgcacaaattttgttcggaaaaaacggagtggaccatatatttgtgcacggtctgcaaacagagtgtctgaagaagacatggccacaaatatgagcagaaggaaaggggttcctcagagatcgcctttgtgctaa

SEQ ID NO: 31 - DNA, 1453 bp, synthetic construct
  source 1..1453: mol_type = other DNA
cttctaacaagcatactacacacccctcctccctccccatccttcataagctctcatacgtctctctctctctcccttcttcaaaacaccccaccccatccaaaacctagaacacacacaacaccagagggctttacaaccgcgcacatgatcatggtaaggactctaaccttcatgttttcatgtgaattctcgtatgaaatcgccatttattgagcaaaaaaaaatatatggaattgggcaacaccagcaggccatgtggaagccgggctggctggaggctctggacacacaaaagttcttcgtggcgtgctcgttccacgagcacgccaagaagaacgagaagaacatatgttgcctagattgctgcacctcgatctgcccgcattgtgtggccgctcatcgggcgcaccgcctcctccaggtgcgccgctacgtgtaccacgatgtggtgcggctggaggacctggagaagcttattgattgctctagtgttcaggtatatcaattatgcacagtgcatgcatgtgactctggtgttgttctgaggttcaagatgcaagaaggcttataattctgtatcaacgtggtgcagtcttatactattaacagctctaaggttgttttcctgaagaagagaccacagaataggcaattcaagggttcagggaatatctgcacctcctgcgacaggagccttcaagaaccgtatttccactgctctctggattgcaaggtgattaactcgaactgatatgctatggttatcctactatgtttcagaaaataaaattcatgctaaatgaggggagcagaataattatgatctaaaaggtcgttaggaagagcaagtatttcgaacttgtttgtcatgcaatattcatgccagttataaaaaaatttgtttcatagtatgcatcattttcaggtagagtatatactacgacagaagaaaaaattgtcagcatatttgcgcccatgcaagaccttgcagcttggccctgatttcttcattcctcatgatgctgatgacgacacaactcactcaacccttgttgatgttgatgagcccatgggatcatcggactcggagaatttgagtgtgccgtgcacaaattttgttcggaaaaaacggagtggaccatatatttgtgcacggtctgcaaacagagtgtctgaagaagacatggccacaaatatgagcagaaggaaaggggttcctcagagatcgcctttgtgctaataaattcaatagttaattattttcccttttctttacttctttcatgttctttatctcttggactaaactggtgcaacaatgatagaattttgtctcagtactaccatgtgattaaaatacatcactagaggcaattgtactcaattcttcaagttggtgcaccagtttatatatatgtatgctttaacatagttcatgttatcccctttt

SEQ ID NO: 32 - DNA, 1068 bp, synthetic construct
  source 1..1068: mol_type = other DNA
cttctaacaagcatactacacacccctcctccctccccatccttcataagctctcatacgtctctctctctctcccttcttcaaaacaccccaccccatccaaaacctagaacacacacaacaccagagggctttacaaccgcgcacatgatcatggccatgtggaagccgggctggctggaggctctggacacacaaaagttcttcgtggcgtgctcgttccacgagcacgccaagaagaacgagaagaacatatgttgcctagattgctgcacctcgatctgcccgcattgtgtggccgctcatcgggcgcaccgcctcctccaggtgcgccgctacgtgtaccacgatgtggtgcggctggaggacctggagaagcttattgattgctctagtgttcagtcttatactattaacagctctaaggttgttttcctgaagaagagaccacagaataggcaattcaagggttcagggaatatctgcacctcctgcgacaggagccttcaagaaccgtatttccactgctctctggattgcaaggtagagtatatactacgacagaagaaaaaattgtcagcatatttgcgcccatgcaagaccttgcagcttggccctgatttcttcattcctcatgatgctgatgacgacacaactcactcaacccttgttgatgttgatgagcccatgggatcatcggactcggagaatttgagtgtgccgtgcacaaattttgttcggaaaaaacggagtggaccatatatttgtgcacggtctgcaaacagagtgtctgaagaagacatggccacaaatatgagcagaaggaaaggggttcctcagagatcgcctttgtgctaataaattcaatagttaattattttcccttttctttacttctttcatgttctttatctcttggactaaactggtgcaacaatgatagaattttgtctcagtactaccatgtgattaaaatacatcactagaggcaattgtactcaattcttcaagttggtgcaccagtttatatatatgtatgctttaacatagttcatgttatcccctttt

SEQ ID NO: 33 - protein, 236 aa, Zea mays; identical to SEQ ID NO: 28

SEQ ID NO: 34 - RNA, 1476 nt, Zea mays
  source 1..1476: mol_type = other RNA
atgcacacggacgacgtctttctggcatcgctactaccagacagagaggacaggggaggtcagactcagaaacgctcgctcacgacggcagcagccatggcagcatcgtgcggcggccgcgtggtggtcttcccgttcccgttccagggccacttcaacccggtgatgcgcctggcccgcgcgctgcacgcccggggcgtcgggatcaccgtgttccacaccgccggcgcgcgggcgccggacccggccgactaccccgccgactaccgcttcgtgcccgtgcccgtggaggtggccccggagctgatggcgtccgaggacatcgccgccatcgtcacggcgctcaacgccgcctgcgaggcgcccttccgggaccgcctctcggcgctgctctccgccgccgacggcgaggcgggggaggcggggggccgcgtccgctgcgtcctcaccgacgtcagctgggacgccgtgctgtcggcggcccgcggcctcggcgtgcccgcgctcggcgtcatgacggccagcgccgccacgttccgcgtctacatggcgtaccgcaccttggtcgacaagggatacctgccggtgagagaggagcgcaaggacgacgcggtcgccgagctacccccgtaccgcgtgaaggacctgctgcggcacgagacgtgcgacctggaggagttcgcggacctgctgggccgcgtggtcgcggcggcgcggctgtcctcggggctcatcttccacacgttccccttcatcgaggccggcacgctgggcgagatccgggacgacatgtcggtgccggtgtacgccgtggcgccgctcaacaagctggtgccggcggccacggccagcctgcacggcgaggtgcaggcggaccggggctgcctgcgctggctggacgcgcagcgggcgcgctccgtgctgtacgtgagcttcgggagcatggcggccatggacccgcacgagttcgtggagctggcgtgggggctggccgacgccggccgccccttcgtgtgggtggtccggcccaacctcatccgcggcttcgagtcgggcgcgctgcccgacggcgtggaggaccgggtgcgcggccgcggcgtcgtcgtcagctgggcgccgcaggaggaggtgctcgcgcacccggccgtgggcggcttcttcacccactgcggctggaactccaccgtggaggccgtgtcggagggcgtgcccatgatctgccacccgcgccacggggaccagtacggcaacgcgaggtacgtgtgccacgtctggaaggtgggcacggaggtcgccggggaccagctggagagaggggagatcaaggccgccatcgacaggctcatgggcggcagcgaggaaggggagggcatcaggaagaggatgaacgagctcaagatcgctgcggacaagggcatcgatgaatctgctgggtcggatttaactaatttggttcatctcataaactcctactga

SEQ ID NO: 35 - RNA, 1476 nt, synthetic construct
  source 1..1476: mol_type = other RNA
atgcacaccgacgacgtgttcctcgcaagcttgctgccagatcgcgaggaccgcggtggtcagactcaaaaacggagtctgaccaccgcggccgcgatggcagcaagctgcgggggacgcgtcgtggtgttccccttccccttccagggccactttaatcccgtcatgcgcctagcgcgggcactccatgcccgcggggtgggcatcaccgtgttccacaccgccggtgcgcgcgcaccggacccggccgattaccccgccgattaccggtttgtccccgtccccgtggaggtagcgccggaattgatggccagcgaggatatcgcggccatcgtgacggccctgaacgcggcctgcgaggccccgttcagggaccgtctttcggcgctgctctccgccgccgacggcgaggcgggggaggcggggggccgcgtccgctgcgtcctcaccgacgtcagctgggacgccgtgctgtcggcggcccgcggcctcggcgtgcccgcgctcggcgtcatgacggccagcgccgccacgttccgcgtctacatggcgtaccgcaccttggtcgacaagggatacctgccggtgagagaggagcgcaaggacgacgcggtcgccgagctacccccgtaccgcgtgaaggacctgctgcggcacgagacgtgcgacctggaggagttcgcggacctgctgggccgcgtggtcgcggcggcgcggctgtcctcggggctcatcttccacacgttccccttcatcgaggccggcacgctgggcgagatccgggacgacatgtcggtgccggtgtacgccgtggcgccgctcaacaagctggtgccggcggccacggccagcctgcacggcgaggtgcaggcggaccggggctgcctgcgctggctggacgcgcagcgggcgcgctccgtgctgtacgtgagcttcgggagcatggcggccatggacccgcacgagttcgtggagctggcgtgggggctggccgacgccggccgccccttcgtgtgggtggtccggcccaacctcatccgcggcttcgagtcgggcgcgctgcccgacggcgtggaggaccgggtgcgcggccgcggcgtcgtcgtcagctgggcgccgcaggaggaggtgctcgcgcacccggccgtgggcggcttcttcacccactgcggctggaactccaccgtggaggccgtgtcggagggcgtgcccatgatctgccacccgcgccacggggaccagtacggcaacgcgaggtacgtgtgccacgtctggaaggtgggcacggaggtcgccggggaccagctggagagaggggagatcaaggccgccatcgacaggctcatgggcggcagcgaggaaggggagggcatcaggaagaggatgaacgagctcaagatcgctgcggacaagggcatcgatgaatctgctgggtcggatttaactaatttggttcatctcataaactcctactga

SEQ ID NO: 36 - DNA, 2047 bp, synthetic construct
  source 1..2047: mol_type = other DNA
gagcatcggcacacgtagcattggacacggaccggcagcatcagatgcacaccgacgacgtgtacgtttggaatttaagtaatttttttagtggaagaattatatcacccaccacctgctgttggatcgagcgtgtgcgtgtcggtgtctgtgtctcatcatcgccgtcgtcatctgtttaatactactagtcacatgggcatttgctgtaggttcctcgcaagcttgctgccagatcgcgaggaccgcggtggtcagactcaaaaacggagtctgaccaccgcggccgcgatggcagcaagctgcgggggacgcgtcgtggtgttccccttccccttccagggccactttaatcccgtcatgcgcctagcgcgggcactccatgcccgcggggtgggcatcaccgtgttccacaccgccggtgcgcgcgcaccggacccggccgattaccccgccgattaccggtttgtccccgtccccgtggaggtagcgccggaattgatggccagcgaggatatcgcggccatcgtgacggccctgaacgcggcctgcgaggccccgttcagggaccgtctttcggcgctgctctccgccgccgacggcgaggcgggggaggcggggggccgcgtccgctgcgtcctcaccgacgtcagctgggacgccgtgctgtcggcggcccgcggcctcggcgtgcccgcgctcggcgtcatgacggccagcgccgccacgttccgcgtctacatggcgtaccgcaccttggtcgacaagggatacctgccggtgagaggtgagtccgtccaagtcgatttcgtcggcggcggcgaggttagagttagattagatctgcctacttgcttatacataatggtttggattggatgcgtgaatcgatctaatcaaatcagaggagcgcaaggacgacgcggtcgccgagctacccccgtaccgcgtgaaggacctgctgcggcacgagacgtgcgacctggaggagttcgcggacctgctgggccgcgtggtcgcggcggcgcggctgtcctcggggctcatcttccacacgttccccttcatcgaggccggcacgctgggcgagatccgggacgacatgtcggtgccggtgtacgccgtggcgccgctcaacaagctggtgccggcggccacggccagcctgcacggcgaggtgcaggcggaccggggctgcctgcgctggctggacgcgcagcgggcgcgctccgtgctgtacgtgagcttcgggagcatggcggccatggacccgcacgagttcgtggagctggcgtgggggctggccgacgccggccgccccttcgtgtgggtggtccggcccaacctcatccgcggcttcgagtcgggcgcgctgcccgacggcgtggaggaccgggtgcgcggccgcggcgtcgtcgtcagctgggcgccgcaggaggaggtgctcgcgcacccggccgtgggcggcttcttcacccactgcggctggaactccaccgtggaggccgtgtcggagggcgtgcccatgatctgccacccgcgccacggggaccagtacggcaacgcgaggtacgtgtgccacgtctggaaggtgggcacggaggtcgccggggaccagctggagagaggggagatcaaggccgccatcgacaggctcatgggcggcagcgaggaaggggagggcatcaggaagaggatgaacgagctcaagatcgctgcggacaagggcatcgatgaatctgctgggtcggatttaactaatttggttcatctcataaactcctactgatcgatccttgtattgggtaccatgttccggatgatgagttggaataaatcaaaacttgtgttccagtcgatctacgcttctcatcattcatcatcatcaatggacaacaaaacctacagctacagggtttagaacattaacagacttgttttcgagcttcctcaggattcaacccagttgtgctagagcatcgtctctaacagattatggatgttctcctgatattagtaacagattatggatgttctcctgatatta

SEQ ID NO: 37 - protein, 638 aa, Zea mays
MATAAAASTALTGATTAAPKARRRAHLLATRRALAAPIRCSAASPAMPMAPPATPLRPWGPTDPRKGADILVESLERCGVRDVFAYPGGASMEIHQALTRSPVIANHLFRHEQGEAFAASGYARSSGRVGVCIATSGPGATNLVSALADALLDSVPMVAITGQVPRRMIGTDAFQETPIVEVTRSITKHNYLVLDVDDIPRVVQEAFFLASSGRPGPVLVDIPKDIQQQMAVPVWDKPMSLPGYIARLPKPPATELLEQVLRLVGESRRPVLYVGGGCAASGEELRRFVELTGIPVTTTLMGLGNFPSDDPLSLRMLGMHGTVYANYAVDKADLLLALGVRFDDRVTGKIEAFASRAKIVHVDIDPAEIGKNKQPHVSICADVKLALQGMNALLEGSTSKKSFDFGSWNDELDQQKREFPLGYKTSNEEIQPQYAIQVLDELTKGEAIIGTGVGQHQMWAAQYYTYKRPRQWLSSAGLGAMGFGLPAAAGASVANPGVTVVDIDGDGSFLMNVQELAMIRIENLPVKVFVLNNQHLGMVVQWEDRFYKANRAHTYLGNPENESEIYPDFVTIAKGFNIPAVRVTKKNEVRAAIKKMLETPGPYLLDIIVPHQEHVLPMIPSGGAFKDMILDGDGRTVY

SEQ ID NO: 38 - protein, 638 aa, synthetic construct
MATAAAASTALTGATTAAPKARRRAHLLATRRALAAPIRCSAASPAMPMAPPATPLRPWGPTDPRKGADILVESLERCGVRDVFAYPGGASMEIHQALTRSPVIANHLFRHEQGEAFAASGYARSSGRVGVCIATSGPGATNLVSALADALLDSVPMVAITGQVARRMIGTDAFQETPIVEVTRSITKHNYLVLDVDDIPRVVQEAFFLASSGRPGPVLVDIPKDIQQQMAVPVWDKPMSLPGYIARLPKPPATELLEQVLRLVGESRRPVLYVGGGCAASGEELRRFVELTGIPVTTTLMGLGNFPSDDPLSLRMLGMHGTVYANYAVDKADLLLALGVRFDDRVTGKIEAFASRAKIVHVDIDPAEIGKNKQPHVSICADVKLALQGMNALLEGSTSKKSFDFGSWNDELDQQKREFPLGYKTSNEEIQPQYAIQVLDELTKGEAIIGTGVGQHQMWAAQYYTYKRPRQWLSSAGLGAMGFGLPAAAGASVANPGVTVVDIDGDGSFLMNVQELAMIRIENLPVKVFVLNNQHLGMVVQLEDRFYKANRAHTYLGNPENESEIYPDFVTIAKGFNIPAVRVTKKNEVRAAIKKMLETPGPYLLDIIVPHQEHVLPMIPSGGAFKDMILDGDGRTVY

SEQ ID NO: 39 - RNA, 1917 nt, Zea mays
  source 1..1917: mol_type = other RNA
atggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgctgctcgattccgtccccatggtcgccatcacgggacaggtgccgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccggggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcccggcgccctgttctttatgttggcggtggctgcgcagcatctggtgaggagttgcgacgctttgtggagctgactggaatcccggtcacaactactcttatgggcctcggcaacttccccagcgacgacccactgtctctgcgcatgctaggtatgcatggcacggtgtatgcaaattatgcagtggataaggccgatctgttgcttgcacttggtgtgcggtttgatgatcgtgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggccaacccaggtgttactgttgttgacatcgatggagatggtagctttctcatgaacgttcaggagctagctatgatccgaattgagaacctcccggtgaaggtctttgtgctaaacaaccagcacctggggatggtggtgcagtgggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactga

SEQ ID NO: 40 - RNA, 1917 nt, synthetic construct
  source 1..1917: mol_type = other RNA
atggccacggccgccacggccgcagcggcgttgacgggcgcgacgaccgcgacgccaaagtcgcggcgtcgcgcccatcacctggcgacacgccgggcgctggcggcgccgattcggtgtagcgcgctatcgcgcgctacaccgacggcgcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccggggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcccggcgccctgttctttatgttggcggtggctgcgcagcatctggtgaggagttgcgacgctttgtggagctgactggaatcccggtcacaactactcttatgggcctcggcaacttccccagcgacgacccactgtctctgcgcatgctaggtatgcatggcacggtgtatgcaaattatgcagtggataaggccgatctgttgcttgcacttggtgtgcggtttgatgatcgtgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggccaacccaggtgttactgttgttgacatcgatggagatggtagctttctcatgaacgttcaggagctagctatgatccgaattgagaacctcccggtgaaggtctttgtgctaaacaaccagcacctggggatggtggtgcagctggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactga

SEQ ID NO: 41 - DNA, 2507 bp, synthetic construct
  source 1..2507: mol_type = other DNA
ccattcccatcaccatctgagccacacatcctctgaacaaaagcagggaggcctctacgcacatccccctttctcccactccgtgtccgtggcacccaccccaaaccctcgcgccgcctccgagacagccgccgcaaccatggccacggccgccacggccgcagcggcgttgacgggcgcgacgaccgcgacgccaaagtcgcggcgtcgcgcccatcacctggcgacacgccgggcgctggcggcgccgattcggtgtagcgcgctatcgcgcgctacaccgacggcgcccccggccaccccgctccggccgtggggccccaccgatccccgcaagggcgccgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgcggcctccggctacgcgcgctcctcgggccgcgtcggcgtctgcatcgccacctccggccccggcgccaccaaccttgtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccggggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcccggcgccctgttctttatgttggcggtggctgcgcagcatctggtgaggagttgcgacgctttgtggagctgactggaatcccggtcacaactactcttatgggcctcggcaacttccccagcgacgacccactgtctctgcgcatgctaggtatgcatggcacggtgtatgcaaattatgcagtggataaggccgatctgttgcttgcacttggtgtgcggtttgatgatcgtgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggccaacccaggtgttactgttgttgacatcgatggagatggtagctttctcatgaacgttcaggagctagctatgatccgaattgagaacctcccggtgaaggtctttgtgctaaacaaccagcacctggggatggtggtgcagctggaggacaggttctataaggccaacagagcgcacacatacttgggaaacccagagaatgaaagtgagatatatccagatttcgtgacgatcgccaaagggttcaacattccagcggtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgatctaaaatccagcaagcaactgatctaaaatccagcaagcaccgcctccctgctagtacaagggtgatatgtttttatctgtgtgatgttctcctgtattctatctttttttgtaggccgtcagctatctgttatggtaatcctatgtagcttccgaccttgtaattgtgtagtctgttgttttccttctggcatgtgtcataagagatcatttaagtgccttttgctacatataaataagataataagcactgctatgcagtggttctgaattggcttctgttgccaaatttaagtgtccaactggtccttgcttttgttttcgctatttttttccttttttagttattattatattggtaatttcaactcaacatatgatgtatggaataatgctagggctgcaatttcaaactattttacaaaccagaatggcattttcgtggtttga

SEQ ID NO: 42 - protein, 638 aa, Zea mays
MATAATAAAALTGATTATPKSRRRAHHLATRRALAAPIRCSALSRATPTAPPATPLRPWGPNEPRKGSDILVEALERCGVRDVFAYPGGASMEIHQALTRSPVIANHLFRHEQGEAFAASGYARSSGRVGVCIATSGPGATNLVSALADALLDSVPIVAITGQVPRRMIGTDAFQETPIVEVTRSITKHNYLVLDVDDIPRVVQEAFFLASSGRPGPVLVDIPKDIQQQMAVPAWDTPMSLPGYIARLPKPPATEFLEQVLRLVGESRRPVLYVGGGCAASGEELCRFVELTGIPVTTTLMGLGNFPSDDPLSLRMLGMHGTVYANYAVDKADLLLAFGVRFDDRVTGKIEAFAGRAKIVHIDIDPAEIGKNKQPHVSICADVKLALQGMNTLLEGSTSKKSFDFGSWHDELDQQKREFPLGYKIFNEEIQPQYAIQVLDELTKGKAIIATGVGQHQMWAAQYYTYKRPRQWLSSAGLGAMGFGLPAAAGAAVANPGVTVVDIDGDGSFLMNIQELAMIRIENLPVKVFVLNNQHLGMVVQWEDRFYKANRAHTFLGNPENESEIYPDFVAIAKGFNIPAVRVTKKSEVHAAIKKMLEAPGPYLLDIIVPHQEHVLPMIPSGGAFKDMILDGDGRTVY

SEQ ID NO: 43 - protein, 638 aa, synthetic construct
MATAATAAAALTGATTATPKSRRRAHHLATRRALAAPIRCSALSRATPTAPPATPLRPWGPNEPRKGSDILVEALERCGVRDVFAYPGGASMEIHQALTRSPVIANHLFRHEQGEAFAASGYARSSGRVGVCIATSGPGATNLVSALADALLDSVPIVAITGQVARRMIGTDAFQETPIVEVTRSITKHNYLVLDVDDIPRVVQEAFFLASSGRPGPVLVDIPKDIQQQMAVPAWDTPMSLPGYIARLPKPPATEFLEQVLRLVGESRRPVLYVGGGCAASGEELCRFVELTGIPVTTTLMGLGNFPSDDPLSLRMLGMHGTVYANYAVDKADLLLAFGVRFDDRVTGKIEAFAGRAKIVHIDIDPAEIGKNKQPHVSICADVKLALQGMNTLLEGSTSKKSFDFGSWHDELDQQKREFPLGYKIFNEEIQPQYAIQVLDELTKGKAIIATGVGQHQMWAAQYYTYKRPRQWLSSAGLGAMGFGLPAAAGAAVANPGVTVVDIDGDGSFLMNIQELAMIRIENLPVKVFVLNNQHLGMVVQLEDRFYKANRAHTFLGNPENESEIYPDFVAIAKGFNIPAVRVTKKSEVHAAIKKMLEAPGPYLLDIIVPHQEHVLPMIPSGGAFKDMILDGDGRTVY

SEQ ID NO: 44 - RNA, 1916 nt, Zea mays
  source 1..1916: mol_type = other RNA
atggccaccgccgccaccgcggccgccgcgctcaccggcgccactaccgctacgcccaagtcgaggcgccgagcccaccacttggccacccggcgcgccctcgccgcgcccatcaggtgctcagcgttgtcacgcgccacgccgacggctcccccggccactccgctacgtccgtggggccccaacgagccccgcaagggctccgacatcctcgtcgaggctctcgagcgctgtggcgtccgtgacgtcttcgcctaccccggcggcgcatccatggagatccaccaggcactcacccgctcccccgtcatcgccaaccacctcttccgccacgaacaaggggaggccttcgccgcctccggctacgcgcgctcctcgggccgcgttggcgtctgcatcgccacctccggccccggcgccaccaacctagtctctgcgctcgcagacgcgttgctcgactccgtccccattgtcgccatcacgggacaggtggcgcgacgcatgattggcaccgacgcctttcaggagacgcccatcgtcgaggtcacccgctccatcaccaagcacaactacctggtcctcgacgtcgacgacatcccccgcgtcgtgcaggaggccttcttcctcgcatcctctggtcgcccggggccggtgcttgttgacatccccaaggacatccagcagcagatggcggtgccggcctgggacacgcccatgagtctgcctgggtacatcgcgcgccttcccaagcctcccgcgactgaatttcttgagcaggtgctgcgtcttgttggtgaatcacggcgccctgttctttatgttggcggtggctgtgcagcatcaggtgaggagttgtgccgctttgtggagttgactggaatcccagtcacaactactcttatgggccttggcaacttccccagcgacgacccactgtcactgcgcatgcttggtatgcatggccagtgtatgcaaattatgcagtggataaggccgatctgttgcttgcatttggtgtgcggtttgatgatcgtgtgacagggaaaattgaggcttttgcaggcagagctaagattgtgcacattgatattgatcctgctgagattggcaagaacaagcagccacatgtgtccatctgtgcagatgttaagcttgctttgcagggcatgaatactcttctggaaggaagcacatcaaagaagagctttgacttcggctcatggcatgatgaattggatcagcaaaagcgggagtttccccttgggtataaaatcttcaatgaggaaatccagccacaatatgctattcaggttcttgatgagttgacgaaggggaaggccatcattgccacaggtgttgggcagcaccagatgtgggcggcacagtattacacttacaagcggccaaggcagtggctgtcttcagctggtcttggggctatgggatttggtttgccggctgctgctggtgctgctgtggccaacccaggtgtcactgttgttgacatcgacggagatggtagcttcctcatgaacattcaggagctagctatgatccgtattgagaacctcccagtcaaggtctttgtgctaaacaaccagcacctcgggatggtggtgcagctggaggacaggttctataaggccaatagagcacacacattcttgggaaacccagagaacgaaagtgagatatatccagattttgtggcaattgccaaagggttcaacattccagcagtccgtgtgacaaagaagagcgaagtccatgcagcaatcaagaagatgcttgaggctccagggccgtacctcttggatataatcgtcccgcaccaggagcatgtgttgcctatgatccctagtggtggggctttcaaggatatgatcctggatggtgatggcaggactgtgtattga

SEQ ID NO: 45 - RNA, 1917 nt, synthetic construct
  source 1..1917: mol_type = other RNA
atggccacggccgccacggccgcagcggcgttgacgggcgcgacgaccgcgacgccaaagtcgcggcgtcgcgcccatcacctggcgacacgccgggcgctggcggcgccgattcggtgtagcgcgctatcgcgcgctacaccgacggcgccgccagcgacgccgctacgtccgtggggccccaacgagccccgcaagggctccgacatcctcgtcgaggctctcgagcgctgtggcgtccgtgacgtcttcgcctaccccggcggcgcatccatggagatccaccaggcactcacccgctcccccgtcatcgccaaccacctcttccgccacgaacaaggggaggccttcgccgcctccggctacgcgcgctcctcgggccgcgttggcgtctgcatcgccacctccggccccggcgccaccaacctagtctctgcgctcgcagacgcgttgctcgactccgtccccattgtcgccatcacgggacaggtggcgcgacgcatgattggcaccgacgcctttcaggagacgcccatcgtcgaggtcacccgctccatcaccaagcacaactacctggtcctcgacgtcgacgacatcccccgcgtcgtgcaggaggccttcttcctcgcatcctctggtcgcccggggccggtgcttgttgacatccccaaggacatccagcagcagatggcggtgccggcctgggacacgcccatgagtctgcctgggtacatcgcgcgccttcccaagcctcccgcgactgaatttcttgagcaggtgctgcgtcttgttggtgaatcacggcgccctgttctttatgttggcggtggctgtgcagcatcaggtgaggagttgtgccgctttgtggagttgactggaatcccagtcacaactactcttatgggccttggcaacttccccagcgacgacccactgtcactgcgcatgcttggtatgcatggcacagtgtatgcaaattatgcagtggataaggccgatctgttgcttgcatttggtgtgcggtttgatgatcgtgtgacagggaaaattgaggcttttgcaggcagagctaagattgtgcacattgatattgatcctgctgagattggcaagaacaagcagccacatgtgtccatctgtgcagatgttaagcttgctttgcagggcatgaatactcttctggaaggaagcacatcaaagaagagctttgacttcggctcatggcatgatgaattggatcagcaaaagcgggagtttccccttgggtataaaatcttcaatgaggaaatccagccacaatatgctattcaggttcttgatgagttgacgaaggggaaggccatcattgccacaggtgttgggcagcaccagatgtgggcggcacagtattacacttacaagcggccaaggcagtggctgtcttcagctggtcttggggctatgggatttggtttgccggctgctgctggtgctgctgtggccaacccaggtgtcactgttgttgacatcgacggagatggtagcttcctcatgaacattcaggagctagctatgatccgtattgagaacctcccagtcaaggtctttgtgctaaacaaccagcacctcgggatggtggtgcagctggaggacaggttctataaggccaatagagcacacacattcttgggaaacccagagaacgaaagtgagatatatccagattttgtggcaattgccaaagggttcaacattccagcagtccgtgtgacaaagaagagcgaagtccatgcagcaatcaagaagatgcttgaggctccagggccgtacctcttggatataatcgtcccgcaccaggagcatgtgttgcctatgatccctagtggtggggctttcaaggatatgatcctggatggtgatggcaggactgtgtattga

SEQ ID NO: 46 - DNA, 2387 bp, synthetic construct
  source 1..2387: mol_type = other DNA
ccccctttcccacaatcccactccgtgccaggtgccaccctccccaagccctcgcgccgcctccgagacagccgcccgcaaccatggccacggccgccacggccgcagcggcgttgacgggcgcgacgaccgcgacgccaaagtcgcggcgtcgcgcccatcacctggcgacacgccgggcgctggcggcgccgattcggtgtagcgcgctatcgcgcgctacaccgacggcgccgccagcgacgccgctacgtccgtggggccccaacgagccccgcaagggctccgacatcctcgtcgaggctctcgagcgctgtggcgtccgtgacgtcttcgcctaccccggcggcgcatccatggagatccaccaggcactcacccgctcccccgtcatcgccaaccacctcttccgccacgaacaaggggaggccttcgccgcctccggctacgcgcgctcctcgggccgcgttggcgtctgcatcgccacctccggccccggcgccaccaacctagtctctgcgctcgcagacgcgttgctcgactccgtccccattgtcgccatcacgggacaggtggcgcgacgcatgattggcaccgacgcctttcaggagacgcccatcgtcgaggtcacccgctccatcaccaagcacaactacctggtcctcgacgtcgacgacatcccccgcgtcgtgcaggaggccttcttcctcgcatcctctggtcgcccggggccggtgcttgttgacatccccaaggacatccagcagcagatggcggtgccggcctgggacacgcccatgagtctgcctgggtacatcgcgcgccttcccaagcctcccgcgactgaatttcttgagcaggtgctgcgtcttgttggtgaatcacggcgccctgttctttatgttggcggtggctgtgcagcatcaggtgaggagttgtgccgctttgtggagttgactggaatcccagtcacaactactcttatgggccttggcaacttccccagcgacgacccactgtcactgcgcatgcttggtatgcatggcacagtgtatgcaaattatgcagtggataaggccgatctgttgcttgcatttggtgtgcggtttgatgatcgtgtgacagggaaaattgaggcttttgcaggcagagctaagattgtgcacattgatattgatcctgctgagattggcaagaacaagcagccacatgtgtccatctgtgcagatgttaagcttgctttgcagggcatgaatactcttctggaaggaagcacatcaaagaagagctttgacttcggctcatggcatgatgaattggatcagcaaaagcgggagtttccccttgggtataaaatcttcaatgaggaaatccagccacaatatgctattcaggttcttgatgagttgacgaaggggaaggccatcattgccacaggtgttgggcagcaccagatgtgggcggcacagtattacacttacaagcggccaaggcagtggctgtcttcagctggtcttggggctatgggatttggtttgccggctgctgctggtgctgctgtggccaacccaggtgtcactgttgttgacatcgacggagatggtagcttcctcatgaacattcaggagctagctatgatccgtattgagaacctcccagtcaaggtctttgtgctaaacaaccagcacctcgggatggtggtgcagctggaggacaggttctataaggccaatagagcacacacattcttgggaaacccagagaacgaaagtgagatatatccagattttgtggcaattgccaaagggttcaacattccagcagtccgtgtgacaaagaagagcgaagtccatgcagcaatcaagaagatgcttgaggctccagggccgtacctcttggatataatcgtcccgcaccaggagcatgtgttgcctatgatccctagtggtggggctttcaaggatatgatcctggatggtgatggcaggactgtgtattgatctaaagttcagcaagcactgcctacctgcctatctttgacatgcatgagctagtacaagtgtgatatgtttttatcaatgtgatggtactctgttatggtaatcttaagtagcatccaaccctgtgtgtagtatgttgttttcgtgttggcatatgtttcagaagccatcatgtaagtgccttttactacatataaataaggtaataagcattgttatgcaccggttctgaattggtcttcttttgccaaatataggtcctgtttgatacctatagctctagaaaatttggtgtggttggtggagcaggccattaggtgttccataaaatagtggagttgtaagactttagaagacatttagataaattattttatttattattta

SEQ ID NO: 47 - protein, 463 aa, Zea mays; identical to SEQ ID NO: 1

SEQ ID NO: 48 - RNA, 1392 nt, Zea mays; identical to SEQ ID NO: 4
  source 1..1392: mol_type = other RNA

SEQ ID NO: 49 - RNA, 1392 nt, synthetic construct
  source 1..1392: mol_type = other RNA
atgaggccgcgcctcccgccgaacgttccgtcactccccagctccttgagtctcttggcgaactccctgagtagccccgtgaccaacaccccgactcggcccgacagcttccctgcatacttgcagctggctcacctaatggtgagccaggaacgacaggagccggcggtgccgtccagctcgtcgtcgtccgcaaagcgggcggcgacgagcatggacgcaagcccggccccgccgctcctgctgcgtgcacccaccccatcgccctcgatagacctgcccgcgggtaaagacaaagcggacgccgccgcctccaaagcgggggcggcggtgttcgacctgcggcgtgagccgaagatcccggcaccgttcctctggccccaggaggaggcgcgaccctcgtcggccgcagagctggaagtgcccatggtggacgtaggcgtgctgcgaaatggggaccgcgcggggctacgccgcgcggctgcccaggtcgcagccgcctgcgccacccatgggttcttccaggtctgcggccacggggtcgacgccgccctggggcgagcggcactcgacggggcctctgacttcttccgcctacctttggcggagaagcagagggcccgtcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 50 - DNA, 2841 bp, synthetic construct
  source 1..2841: mol_type = other DNA
tgtcgggtcccacatgaggccgcgcctcccgccgaacgttccgtcactccccagctccttgagtctcttggcgaactccctgagtagccccgtgaccaacaccccgactcggcccgacagcttccctgcatacttgcagctggctcacctaatggtgagccaggaacgacaggagccggcggtgccgtccagctcgtcgtcgtccgcaaagcgggcggcgacgagcatggacgcaagcccggccccgccgctcctgctgcgtgcacccaccccatcgccctcgatagacctgcccgcgggtaaagacaaagcggacgccgccgcctccaaagcgggggcggcggtgttcgacctgcggcgtgagccgaagatcccggcaccgttcctctggccccaggaggaggcgcgaccctcgtcggccgcagagctggaagtgcccatggtggacgtaggcgtgctgcgaaatggggaccgcgcggggctacgccgcgcggctgcccaggtcgcagccgcctgcgccacccatgggttcttccaggtctgcggccacggggtcgacgccgccctggggcgagcggcactcgacggggcctctgacttcttccgcctacctttggcggagaagcagagggcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaactgatttctgtgaa

SEQ ID NO: 51 - protein, 286 aa, Zea mays
MHIREEEATPSTVTGIMSTLTSAGQQKLKEPISPGGGSASVAGSAAERNNGGRGKGKTEIKRIENTTNRQVTFCKRRNGLLKKAYELSVLCDAEVALIVFSSRGRLYEYANNSVKGTIERYKKATSDNSSAAGTIAEVTIQHYKQESARLRQQIVNLQNSNRALIGDSITTMSHKELKHLETRLDKALGKIRAKKNDVLCSEVEYMQRREMELQNDNLYLRSRVDENERAQQTANMMGAPSTSEYQQHGFTPYDPIRSFLQFNIVQQPQFYSQQEDRKDFNDQGGR

SEQ ID NO: 52 - RNA, 861 nt, synthetic construct
  source 1..861: mol_type = other RNA
atgcatatccgtgaggaggaggccacgccttctaccgtcacgggtatcatgtctacactgacctccgctggtcagcagaagttgaaagagccgatctctcctggtgggggtagtgcttcggttgctggcagtgctgcagagaggaacaatgggggtcgtgggaagggtaagaccgagatcaagcggatcgagaacactacgaaccgccaggtcaccttctgcaagaggcgaaacggcctcttgaagaaggcttatgagctgtcggtactctgcgacgcggaggtggccctcatcgtgttctcgtcgcggggccgcttgtatgagtacgcgaacaatagtgtcaaaggcactattgagcggtacaaaaaggcgacctccgacaatagttccgcagccggaactattgcggaggtcaccatccagcattataagcaggagtccgcccggctccgccagcagattgtcaatctgcagaactccaatcgggcgctgattggtgactccatcactaccatgagccacaaggagctcaagcaccttgagactcgcttggataaggctcttggtaaaatccgagcaaagaagaatgatgttctttgctcggaggtggagtacatgcagcgccgcgagatggagctgcagaatgacaatctgtatttgcggagccgggtggacgaaaacgagcgggcccagcagacggcgaacatgatgggggccccctccacgtcggagtaccagcagcacggttttaccccctacgaccccattcgttcctttctgcagtttaacattgtccagcaaccgcagttctactcccagcaggaggatcggaaagacttcaatgaccagggaggtcggtga

SEQ ID NO: 53 - RNA, 861 nt, synthetic construct
  source 1..861: mol_type = other RNA
atgcacataagagaagaagaagcaacaccatcaacagtaacaggaataatgtcaacactaacatcagcaggacaacaaaaactaaaagaaccaatatcaccaggaggaggatcagcatcagtagcaggatcagcagcagaaagaaacaacggaggaagaggaaaaggaaaaacagaaataaaaagaatagaaaacacaacaaacagacaagtaacattctgcaaaagaagaaacggactactaaaaaaagcatacgaactatcagtactatgcgacgcagaagtagcactaatagtattctcatcaagaggaagactatacgaatacgcaaacaactcagtaaaaggaacaatagaaagatacaaaaaagcaacatcagacaactcatcagcagcaggaacaatagcagaagtaacaatacaacactacaaacaagaatcagcaagactaagacaacaaatagtaaacctacaaaactcaaacagagcactaataggagactcaataacaacaatgtcacacaaagaactaaaacacctagaaacaagactagacaaagcactaggaaaaataagagcaaaaaaaaacgacgtactatgctcagaagtagaatacatgcaaagaagagaaatggaactacaaaacgacaacctatacctaagatcaagagtagacgaaaacgaaagagcacaacaaacagcaaacatgatgggagcaccatcaacatcagaataccaacaacacggattcacaccatacgacccaataagatcattcctacaattcaacatagtacaacaaccacaattctactcacaacaagaagacagaaaagacttcaacgaccaaggaggaagataa

SEQ ID NO: 54 - RNA, 860 nt, Zea mays
  source 1..860: mol_type = other RNA
atgcacatccgagaagaggaggctacaccatccacagtaacaggcatcatgtcgaccctgacttcggcggggcagcagaagctgaaggagcccatatcccctggtggcggctccgcgtcggtcgctgggtccgctgcggagaggaacaacggcggcaggggcaagggcaagactgagatcaagcgcatcgagaacacgaccaacaggcaggtcaccttctgcaagcgccgcaacggcctcctcaagaaggcgtacgagctctccgtgctctgcgacgccgaggtcgcgctcatcgtcttctccagccgcggccgcctctacgagtacgccaacaacagcgtgaagggcaccattgagaggtacaagaaggcaaccagtgacaactccagcgcagctggtacgattgcagaggtcaccattcagcattacaagcaggaatctgctaggctgaggcagcagatcgttaacttgcagaactccaacagggccctgataggtgattctatcacaaccatgagccacaaggaacttaagcacttggagactaggttagacaaagctctcggaaagattagagcaaaaaagaacgatgtgctgtgttctgaagtcgagtacatgcagagaagggaaatggagttgcagaatgacaacttgtacttaaggagccgggttgatgagaatgaaagggcacaacagacagcgaacatgatgggggcaccatcgacaagtgagtatcagcagcacggttttactccttatgatccaataaggagcttcctgcagttcaacatcgtgcagcagcctcagttctattctcagcaggaggaccggaaagacttcaacgaccaaggtggaagata

SEQ ID NO: 55 - RNA, 861 nt, synthetic construct
  source 1..861: mol_type = other RNA
atgcatatccgtgaggaggaggccacgccttctaccgtcacgggtatcatgtctacactgacctccgctggtcagcagaagttgaaagagccgatctctcctggtgggggtagtgcttcggttgctggcagtgctgcagagaggaacaatgggggtcgtgggaagggtaagaccgagatcaagcggatcgagaacactacgaaccgccaggtcaccttctgcaagaggcgaaacggcctcttgaagaaggcttatgagctgtccgtgctctgcgacgccgaggtcgcgctcatcgtcttctccagccgcggccgcctctacgagtacgccaacaacagcgtgaagggcaccattgagaggtacaagaaggcaaccagtgacaactccagcgcagctggtacgattgcagaggtcaccattcagcattacaagcaggaatctgctaggctgaggcagcagatcgttaacttgcagaactccaacagggccctgataggtgattctatcacaaccatgagccacaaggaacttaagcacttggagactaggttagacaaagctctcggaaagattagagcaaaaaagaacgatgtgctgtgttctgaagtcgagtacatgcagagaagggaaatggagttgcagaatgacaacttgtacttaaggagccgggttgatgagaatgaaagggcacaacagacagcgaacatgatgggggcaccatcgacaagtgagtatcagcagcacggttttactccttatgatccaataaggagcttcctgcagttcaacatcgtgcagcagcctcagttctattctcagcaggaggaccggaaagacttcaacgaccaaggtggaagataa

SEQ ID NO: 56 - RNA, 861 nt, synthetic construct; identical to SEQ ID NO: 55
  source 1..861: mol_type = other RNA

SEQ ID NO: 57 - DNA, 8538 bp, Zea mays
  source 1..8538: mol_type = other DNA
ggagcaagaaacactcagaagctgcccagagctaccacccttcttatccccacccctcctcctcctaccttttctccttcagacctcaaaatctgtgtgtctcctgccgcggctagctgataggaacaagagcatgcacatccgagaagaggaggctacaccatccacagtaacaggcatcatggtgatttcttgttcttcccattggcttctttaatttggagcagatccacctctctgtgtcgttgtcgtctcctagcaattcttgagcgtggttctcttgtgtgcagtcgaccctgacttcggcggggcagcagaagctgaaggagcccatatcccctggtggcggctccgcgtcggtcgctgggtccgctgcggagaggaacaacggcggcaggggcaagggcaagactgagatcaagcgcatcgagaacacgaccaacaggcaggtcaccttctgcaagcgccgcaacggcctcctcaagaaggcgtacgagctctccgtgctctgcgacgccgaggtcgcgctcatcgtcttctccagccgcggccgcctctacgagtacgccaacaacaggtctcttctttctctccccagctctgtgcatctctctctctgtgtggtactagctagctagtaccgcaatataatggtagtaggcgctactacgacgaatcaccttcgctccaagaaatggtaggctacatatataagtgatgagtttgtcgtcccgtctagctagctagacacatgatgtgtatttcgacgacgctctccgagaattgaagcagcaagttagctcccagctttcttgcttgcaaagaggaaagctgcatctcgtttgctgctacgactacatgcgttctgtttattaactgtcttgcttattttttgtgctaccgcggagaggggcgaaagaaatcgcaacggtttctttgcgcgcgtgcgtgctccgtcatcctttccggtttctcttgtgttcctgtgaggtgagctagatgtctagatagattcagcgcaggggtatgctttgtgaggtgagcttttctagtgcgcgtgcgtgcaatgggcgggtgccttgcggttgctttggtttggtgtggtggtccgcttcttttcttgctccatttaattatttctttcctttctatgatctatatatccctgaggaatatcagtgaagacttattagatctctcatacctcatgatacatagcaagcattgttctcacacaaggaaaaaaaactttttgttgctttggtcatgtatatatgtgcagatctagtaattattatccttcgctcttagatttgggtcactagctggatgtgcgcgcgtgcagtaactcggtaactggatgcaaacttgtgaatgtgagtgaggggaggtaagcatttgagacgtaaaaagatgccaggcgcgcatcctgtcctgctagtgctatatgatgttagggttagggttccatcccatgtgcgtccgtcgtcatcatcaccatctggcctctctctctctcttctctctctctctctctctctctctctctctctctctctctcatgctgtcatacattgtgttttctgtagatgttactgttgtccctgcgtgtgtggtggttctgcaggtctcctcgtcacagccatgtatatctgcgcgcgcgctgtgcactgtgctgcatatgcatatgctaaagtgatgcgcaactgcccatcacgggagctctgtcctctctgccaccacatagcactgtagtttttaggactggcaggaaataaaaggggggtaatgtaatgcagaaggaaacgataacatcaaattgaaaacaaagaagcaacctagaaacagcgtgatagcaaattaaatttccttaggcatttgtgcttattttttaagatttctttatcacatccttatgttgcacacgtacatgcaatgcaagtggcatgagtgaacgaatatatagattcattacagctttgacaaattggaaccactccatctgattgaatgcgtttgacttgtaaactcaaaggacaatatgtgttgtgtttcttcgcagaaatttgagcattatgattgggttctcgtatcttcaagtgaatatttactgttaatagtaatagtagtgttcatcacactgaaccataaatttgagagaaacaccattttgtaagtaatcaaattacttttcttgaacagattccaaaagaatcaaaatttcatctatattgttgcaatattgaaactaaaaagtagctcggatgcattagtcaacatggtattgcaaatatttgtacaataaaatcatagttttgaaactgttgtatctatatggtgtttgattggttacttgtgtttgcaatgtattcaggcagtgcagattggttgtatttggtttgccagacatatatagtctcttattcatgcttgcactttgctcaaagaaagcacaataatgcatattagctagtatgccagatttggtcgtacttagacaaggtttgcacaatctgatgggatggtcatacaaaaataaaacaacctcttccctttacatatactaaaaatatgagaactcaaatagaacatggatcggaggtagcaataaagtgtgaagaaccaaacatattttttatacactgcatgtgtttctgatcgactcaggttgcattgcataaggtctaatgcagagaagactaatgcaagcaaacaattagagccataacatcttataattgatataaaagtatacctatctatttcatgataattttaaatataaaaggtttgctcaaataaaaatgcaaactataattgattttttttttgctcaaagctcgacggaagccagatgttcgaacgggtgtagctgcaattcatagagataaaacactgtagaaatattagagcgacctgtacgaattaaagtcaatcgacgctatagatatatatttggtctttgagggcgtacggggcaactgaaatgtgtgccaatttgtttgtgtgatgtgaatgctctgtatacggaaccagtagcatgaccagttttccttgccactgccagagttgtatcgggggactacaaggggaagaaagctggcaatgcaagctggtgttggtcagaagggttatggcacccaacggtagggctgaccagttgggtaacagctgagtccatgtcttatctatgcagcactcctttgtgaccaatgggtagtagatgtccactgcttgcaggcaatcatgttgtgggtaatgcatggctactgccaagcatgctcgaggatgctaaggtttttgctatgttcacctacagagtacagaacagcagtacctttgaaattggtagccatccgtctcaaaattcgttctcattttcgaaaatgcaaccattttaaacttacaaatatatataataaaatattaatattacataattagtatcgttagaagatctttgaatctattttcataacaaatatttttaaagatacaaatattacaaatatttttttataaatttaaatcagtcttgacaaactttgacctgcacgaaatgacttagacaactattttagaatggagagagcatataaattatgtgagtcaaagagtttcttagaaataatggttggcactaagtgggttctacattattgcccccataaatatttctttgaagcactgctattaaaatgcttggcctccgcgatggatgttatgagttccaaaaaaagtgacgagttcctgttaatctgtgtatggcaactgcaaaattggcatgtttagaaaatacatatctcgtgtaagcatagggttcaaatttgagtcaagatgcatgattcatgtctcatatttcttttagctagaataaaagtggtatttacatttcctttttgaacgtaagacatttacatggatatgtcatctttttctgacaaatatttagacaactgttttttaccatcaaagctaattaatgtgaataacacatcattttttagattagattgtttaattttttgcaatgaaaaaatcattcctttgaagaatcatgtgatccatctttctcctttcacttccgttgcaccattaacataagttatttaacaatacaatccatatgttcctttaaatttgtttatgtcctatttagtaaaaatatccacttaagcaacacaaagagagctggctcaacttaactgtgaatgcatcgaatccactaactattgcgcttttggttattatcaacaacaaatatttttattaatatatcaagataatctatattttatcagtttctcaatgtttcaagatttaaaaatataggtcttgagttttttttcttgagagggtaattttaccaggatgttttttggttcagttcaaaatttaattcaatcctaatgtttagatctagaccccctttttttaaacagttcttgtaggatttgactttgttgaagaaaacaaggatgaatttttttgctatgctagcttaggagttccgattttcttaaaaagaactgtagtagttattaggtgtggtattacataggttcaaactccatgtatgtgcactacaaacatattatgggtctgtttggtttagctgtagattcctaaaactctgattctagctatgagctgtgagaaagctactatgaactgacaattatgagaaaactgaaattaatttggctgaaacaactgtcaactatagattctataagagcatgacagcttgtaacatagacatcatgaatccataaaatctagggtaggaggtgattctgaattgaactagaggaatctatagtttatctattctactattgtatatgtttgattgagatttcagattttggacagcaaatctagttcagaaagctaaaccaaacataccatataagaactcaagttctatgtagatgcatcagtatttattagaatgctaactcaatggcagttaggatttgattatgctggcaatgcgatagtatacaatattagactattagtgaagagtgaagcagtaatgtaagcaatgcatgtgcttactaaaacgtgataaatcattgttgcaccgaatattaatacacaacattaggatgtgcttgcacttcctgttattatctgtagcattgacgaggaccactctctattttcacatgacactaaataatgtgttaaaatcaactacttctaagtgattgcagcagtatttaccacaccaaattaatatcacaaaatagattatgaaatatattttcataatttaaccatttagagtcctagatgatgttactatttaagtgattgctgggatggctttactttagcatggaagtattaatagtattttgggacgatgcgttagaatattggttactttatgtgctgattgtattgactttcactaatggggctgttctggctagattattaaagccgttcggactgctgctgccgtatagacaaaatactgtagaaaaggtagaagccggtggattaaagccgaaatactgtagaaaaagtagaagccggtggattaaagccgcaacggacaggatgaatgtcaaattgtaatcgacatacttgctagaaggcaatgtatccattataatcgctggaagatgtcaaatttcagttattgtatatgttctatgaatctccattataatttattatgcttgtgacgattgttctattatctgttgtattttttcaagaaaaatatctagttttgccctggaacaattttgtttcatattttcattctttcggcacatacaattatctcacctgattgaatgtatgtgtactaactactaactacagtattatattgtatgtacagcgtgaagggcaccattgagaggtacaagaaggcaaccagtgacaactccagcgcagctggtacgattgcagaggtcaccattcaggtaacatcatgtcgatcagtcaacacatcgtttctgatcctacaagaccctagtcgtagtgttggctaccacaggctatgctgtctattgatgaccctagtgttgtactatgcaacattccttgcaactgtgtgctctacacagccaaaatattttctctgggcgaaatcttcagactggacatgcaccctgtgggttctgggcattacacggcagaaattccagtgacgatactaagagaacctgcttggaccagcatttcatgtggagttgtgcagatatataattgtcctctctggatgtctgggtagctttggttattccatgctccaaatggtagagaggatagcatgcatgttttggacaaaaacgaataaaaccactgaaggctagtaggaaaattcttcttgatgggttttcgtaagatgtggcaattataatattcctctgataaatatatatatggctagtaggaaactttataagtatatatatggcagtcagtttggtgaaatggagttttagtgttgcacatattttcttttccctgaaattgatttcgatgaataaattaattggactacttttgttttttcttgcaattgagaaatgagtacaccatttaattcacattttgcagtcggacctgtttgttatgaatgtcaatatggaacatccactaacaatttggatagaaaccaacagcagcatgaaatattactaaatgaacttttgcaaacttgcagcattacaagcaggaatctgctaggctgaggcagcagatcgttaacttgcagaactccaacaggttcagttcttatagaaaattatcatgtgcgcgagaatacagaaaggtcatttgatgaacttataagttttcagggccctgataggtgattctatcacaaccatgagccacaaggaacttaagcacttggagactaggttagacaaagctctcggaaagattagagcaaaaaaggtactacgtgctatatatctataataatcatatttactgcattatgtgtggaaaaataaatatagtatttccttttacgcaattaatgcggatcaaactctgcagaacgatgtgctgtgttctgaagtcgagtacatgcagagaagggtaaacagcaacaagaacacatttttcctctcacgaacttattgatttactcgacattgaacaaaataaaaacaagaatttcgcattttcaggaaatggagttgcagaatgacaacttgtacttaaggagccgggtaagtaacctgtagttgctaattaagaaggacagtcttagaagcaaaatatggaactgtaattactaaaatgccaagtttaccttaactttgaaaataaaaaatagtgtaacaatttaactgtctcacaatgcaagcataggttgatgagaatgaaagggcacaacagacagcgaacatgatgggggcaccatcgacaagtgagtatcagcagcacggttttactccttatgatccaataaggagcttcctgcagttcaacatcgtgcagcagcctcagttctattctcagcaggaggaccggaaagacttcaacgaccaaggtagattttttttttatatctaccacatttatttgaaatgtatttaaatggacaccctaaggtatttcataataccatgtatatttttctaaatgaaataataatccctccatctcaaattattattcattttaattcttggtttttaagaatgatgatgaatctagacacatacatcaattattaactgtacgaatctattagaagtttaaaatgaattttaatttgggacagagacaatatttattagtcatcacaaaacttggaagccatagggctaggaaaacaaaatcattgcagttagttttaaataaaatgcctaaaccttccacttggctatatatatatatatatatatatatatatatatatatatatatatatatatatatatatatatatatatatatatatatatatatatatatatatatatatatatatatatatatatatatatatataatttcctctccaagtacatgttctgctattctgattccttttaatgtctttaggtggaagataaatattggacctctcaagcttcagtacttatccgtgatgatgcatgactgccagtgagaaactgagctacattactgtggaattatatgtaaagactagtactactgcttcatatatgtgcatgtgcgcacacgcgcacgtagtatgcacaatttcatcccactattatggcttggcaccaactatgtctccttaattatcataggagaaaaataagtcacgcacaaaaaaattctaaagatgaggcgcagtgttggatgactgaaactgaaagataggattctcgtggatggcggtggtcatatatatgtcatgcacatctgatctctgttccaaaatgccacatcttctttcccaacccactcgaatctacaccgagaatatatatgttttattcgtaatgtatagacactattagcaatgatactgtgaactatggttatggtccacaataagaaatgcatatttacctgtg

SEQ ID NO: 58 - DNA, 8538 bp, synthetic construct
  source 1..8538: mol_type = other DNA
ggagcaagaaacactcagaagctgcccagagctaccacccttcttatccccacccctcctcctcctaccttttctccttcagacctcaaaatctgtgtgtctcctgccgcggctagctgataggaacaagagcatgcatatccgtgaggaggaggccacgccttctaccgtcacgggtatcatggtgatttcttgttcttcccattggcttctttaatttggagcagatccacctctctgtgtcgttgtcgtctcctagcaattcttgagcgtggttctcttgtgtgcagtctacactgacctccgctggtcagcagaagttgaaagagccgatctctcctggtgggggtagtgcttcggttgctggcagtgctgcagagaggaacaatgggggtcgtgggaagggtaagaccgagatcaagcggatcgagaacactacgaaccgccaggtcaccttctgcaagaggcgaaacggcctcttgaagaaggcttatgagctgtccgtgctctgcgacgccgaggtcgcgctcatcgtcttctccagccgcggccgcctctacgagtacgccaacaacaggtctcttctttctctccccagctctgtgcatctctctctctgtgtggtactagctagctagtaccgcaatataatggtagtaggcgctactacgacgaatcaccttcgctccaagaaatggtaggctacatatataagtgatgagtttgtcgtcccgtctagctagctagacacatgatgtgtatttcgacgacgctctccgagaattgaagcagcaagttagctcccagctttcttgcttgcaaagaggaaagctgcatctcgtttgctgctacgactacatgcgttctgtttattaactgtcttgcttattttttgtgctaccgcggagaggggcgaaagaaatcgcaacggtttctttgcgcgcgtgcgtgctccgtcatcctttccggtttctcttgtgttcctgtgaggtgagctagatgtctagatagattcagcgcaggggtatgctttgtgaggtgagcttttctagtgcgcgtgcgtgcaatgggcgggtgccttgcggttgctttggtttggtgtggtggtccgcttcttttcttgctccatttaattatttctttcctttctatgatctatatatccctgaggaatatcagtgaagacttattagatctctcatacctcatgatacatagcaagcattgttctcacacaaggaaaaaaaactttttgttgctttggtcatgtatatatgtgcagatctagtaattattatccttcgctcttagatttgggtcactagctggatgtgcgcgcgtgcagtaactcggtaactggatgcaaacttgtgaatgtgagtgaggggaggtaagcatttgagacgtaaaaagatgccaggcgcgcatcctgtcctgctagtgctatatgatgttagggttagggttccatcccatgtgcgtccgtcgtcatcatcaccatctggcctctctctctctcttctctctctctctctctctctctctctctctctctctctctcatgctgtcatacattgtgttttctgtagatgttactgttgtccctgcgtgtgtggtggttctgcaggtctcctcgtcacagccatgtatatctgcgcgcgcgctgtgcactgtgctgcatatgcatatgctaaagtgatgcgcaactgcccatcacgggagctctgtcctctctgccaccacatagcactgtagtttttaggactggcaggaaataaaaggggggtaatgtaatgcagaaggaaacgataacatcaaattgaaaacaaagaagcaacctagaaacagcgtgatagcaaattaaatttccttaggcatttgtgcttattttttaagatttctttatcacatccttatgttgcacacgtacatgcaatgcaagtggcatgagtgaacgaatatatagattcattacagctttgacaaattggaaccactccatctgattgaatgcgtttgacttgtaaactcaaaggacaatatgtgttgtgtttcttcgcagaaatttgagcattatgattgggttctcgtatcttcaagtgaatatttactgttaatagtaatagtagtgttcatcacactgaaccataaatttgagagaaacaccattttgtaagtaatcaaattacttttcttgaacagattccaaaagaatcaaaatttcatctatattgttgcaatattgaaactaaaaagtagctcggatgcattagtcaacatggtattgcaaatatttgtacaataaaatcatagttttgaaactgttgtatctatatggtgtttgattggttacttgtgtttgcaatgtattcaggcagtgcagattggttgtatttggtttgccagacatatatagtctcttattcatgcttgcactttgctcaaagaaagcacaataatgcatattagctagtatgccagatttggtcgtacttagacaaggtttgcacaatctgatgggatggtcatacaaaaataaaacaacctcttccctttacatatactaaaaatatgagaactcaaatagaacatggatcggaggtagcaataaagtgtgaagaaccaaacatattttttatacactgcatgtgtttctgatcgactcaggttgcattgcataaggtctaatgcagagaagactaatgcaagcaaacaattagagccataacatcttataattgatataaaagtatacctatctatttcatgataattttaaatataaaaggtttgctcaaataaaaatgcaaactataattgattttttttttgctcaaagctcgacggaagccagatgttcgaacgggtgtagctgcaattcatagagataaaacactgtagaaatattagagcgacctgtacgaattaaagtcaatcgacgctatagatatatatttggtctttgagggcgtacggggcaactgaaatgtgtgccaatttgtttgtgtgatgtgaatgctctgtatacggaaccagtagcatgaccagttttccttgccactgccagagttgtatcgggggactacaaggggaagaaagctggcaatgcaagctggtgttggtcagaagggttatggcacccaacggtagggctgaccagttgggtaacagctgagtccatgtcttatctatgcagcactcctttgtgaccaatgggtagtagatgtccactgcttgcaggcaatcatgttgtgggtaatgcatggctactgccaagcatgctcgaggatgctaaggtttttgctatgttcacctacagagtacagaacagcagtacctttgaaattggtagccatccgtctcaaaattcgttctcattttcgaaaatgcaaccattttaaacttacaaatatatataataaaatattaatattacataattagtatcgttagaagatctttgaatctattttcataacaaatatttttaaagatacaaatattacaaatatttttttataaatttaaatcagtcttgacaaactttgacctgcacgaaatgacttagacaactattttagaatggagagagcatataaattatgtgagtcaaagagtttcttagaaataatggttggcactaagtgggttctacattattgcccccataaatatttctttgaagcactgctattaaaatgcttggcctccgcgatggatgttatgagttccaaaaaaagtgacgagttcctgttaatctgtgtatggcaactgcaaaattggcatgtttagaaaatacatatctcgtgtaagcatagggttcaaatttgagtcaagatgcatgattcatgtctcatatttcttttagctagaataaaagtggtatttacatttcctttttgaacgtaagacatttacatggatatgtcatctttttctgacaaatatttagacaactgttttttaccatcaaagctaattaatgtgaataacacatcattttttagattagattgtttaattttttgcaatgaaaaaatcattcctttgaagaatcatgtgatccatctttctcctttcacttccgttgcaccattaacataagttatttaacaatacaatccatatgttcctttaaatttgtttatgtcctatttagtaaaaatatccacttaagcaacacaaagagagctggctcaacttaactgtgaatgcatcgaatccactaactattgcgcttttggttattatcaacaacaaatatttttattaatatatcaagataatctatattttatcagtttctcaatgtttcaagatttaaaaatataggtcttgagttttttttcttgagagggtaattttaccaggatgttttttggttcagttcaaaatttaattcaatcctaatgtttagatctagaccccctttttttaaacagttcttgtaggatttgactttgttgaagaaaacaaggatgaatttttttgctatgctagcttaggagttccgattttcttaaaaagaactgtagtagttattaggtgtggtattacataggttcaaactccatgtatgtgcactacaaacatattatgggtctgtttggtttagctgtagattcctaaaactctgattctagctatgagctgtgagaaagctactatgaactgacaattatgagaaaactgaaattaatttggctgaaacaactgtcaactatagattctataagagcatgacagcttgtaacatagacatcatgaatccataaaatctagggtaggaggtgattctgaattgaactagaggaatctatagtttatctattctactattgtatatgtttgattgagatttcagattttggacagcaaatctagttcagaaagctaaaccaaacataccatataagaactcaagttctatgtagatgcatcagtatttattagaatgctaactcaatggcagttaggatttgattatgctggcaatgcgatagtatacaatattagactattagtgaagagtgaagcagtaatgtaagcaatgcatgtgcttactaaaacgtgataaatcattgttgcaccgaatattaatacacaacattaggatgtgcttgcacttcctgttattatctgtagcattgacgaggaccactctctattttcacatgacactaaataatgtgttaaaatcaactacttctaagtgattgcagcagtatttaccacaccaaattaatatcacaaaatagattatgaaatatattttcataatttaaccatttagagtcctagatgatgttactatttaagtgattgctgggatggctttactttagcatggaagtattaatagtattttgggacgatgcgttagaatattggttactttatgtgctgattgtattgactttcactaatggggctgttctggctagattattaaagccgttcggactgctgctgccgtatagacaaaatactgtagaaaaggtagaagccggtggattaaagccgaaatactgtagaaaaagtagaagccggtggattaaagccgcaacggacaggatgaatgtcaaattgtaatcgacatacttgctagaaggcaatgtatccattataatcgctggaagatgtcaaatttcagttattgtatatgttctatgaatctccattataatttattatgcttgtgacgattgttctattatctgttgtattttttcaagaaaaatatctagttttgccctggaacaattttgtttcatattttcattctttcggcacatacaattatctcacctgattgaatgtatgtgtactaactactaactacagtattatattgtatgtacagcgtgaagggcaccattgagaggtacaagaaggcaaccagtgacaactccagcgcagctggtacgattgcagaggtcaccattcaggtaacatcatgtcgatcagtcaacacatcgtttctgatcctacaagaccctagtcgtagtgttggctaccacaggctatgctgtctattgatgaccctagtgttgtactatgcaacattccttgcaactgtgtgctctacacagccaaaatattttctctgggcgaaatcttcagactggacatgcaccctgtgggttctgggcattacacggcagaaattccagtgacgatactaagagaacctgcttggaccagcatttcatgtggagttgtgcagatatataattgtcctctctggatgtctgggtagctttggttattccatgctccaaatggtagagaggatagcatgcatgttttggacaaaaacgaataaaaccactgaaggctagtaggaaaattcttcttgatgggttttcgtaagatgtggcaattataatattcctctgataaatatatatatggctagtaggaaactttataagtatatatatggcagtcagtttggtgaaatggagttttagtgttgcacatattttcttttccctgaaattgatttcgatgaataaattaattggactacttttgttttttcttgcaattgagaaatgagtacaccatttaattcacattttgcagtcggacctgtttgttatgaatgtcaatatggaacatccactaacaatttggatagaaaccaacagcagcatgaaatattactaaatgaacttttgcaaacttgcagcattacaagcaggaatctgctaggctgaggcagcagatcgttaacttgcagaactccaacaggttcagttcttatagaaaattatcatgtgcgcgagaatacagaaaggtcatttgatgaacttataagttttcagggccctgataggtgattctatcacaaccatgagccacaaggaacttaagcacttggagactaggttagacaaagctctcggaaagattagagcaaaaaaggtactacgtgctatatatctataataatcatatttactgcattatgtgtggaaaaataaatatagtatttccttttacgcaattaatgcggatcaaactctgcagaacgatgtgctgtgttctgaagtcgagtacatgcagagaagggtaaacagcaacaagaacacatttttcctctcacgaacttattgatttactcgacattgaacaaaataaaaacaagaatttcgcattttcaggaaatggagttgcagaatgacaacttgtacttaaggagccgggtaagtaacctgtagttgctaattaagaaggacagtcttagaagcaaaatatggaactgtaattactaaaatgccaagtttaccttaactttgaaaataaaaaatagtgtaacaatttaactgtctcacaatgcaagcataggttgatgagaatgaaagggcacaacagacagcgaacatgatgggggcaccatcgacaagtgagtatcagcagcacggttttactccttatgatccaataaggagcttcctgcagttcaacatcgtgcagcagcctcagttctattctcagcaggaggaccggaaagacttcaacgaccaaggtagattttttttttatatctaccacatttatttgaaatgtatttaaatggacaccctaaggtatttcataataccatgtatatttttctaaatgaaataataatccctccatctcaaattattattcattttaattcttggtttttaagaatgatgatgaatctagacacatacatcaattattaactgtacgaatctattagaagtttaaaatgaattttaatttgggacagagacaatatttattagtcatcacaaaacttggaagccatagggctaggaaaacaaaatcattgcagttagttttaaataaaatgcctaaaccttccacttggctatatatatatatatatatatatatatatatatatatatatatatatatatatatatatatatatatatatatatatatatatatatatatatatatatatatatatatatatatatatatatatataatttcctctccaagtacatgttctgctattctgattccttttaatgtctttaggtggaagataaatattggacctctcaagcttcagtacttatccgtgatgatgcatgactgccagtgagaaactgagctacattactgtggaattatatgtaaagactagtactactgcttcatatatgtgcatgtgcgcacacgcgcacgtagtatgcacaatttcatcccactattatggcttggcaccaactatgtctccttaattatcataggagaaaaataagtcacgcacaaaaaaattctaaagatgaggcgcagtgttggatgactgaaactgaaagataggattctcgtggatggcggtggtcatatatatgtcatgcacatctgatctctgttccaaaatgccacatcttctttcccaacccactcgaatctacaccgagaatatatatgttttattcgtaatgtatagacactattagcaatgatactgtgaactatggttatggtccacaataagaaatgcatatttacctgtg

SEQ ID NO: 59 - DNA, 8538 bp, synthetic construct; identical to SEQ ID NO: 58
  source 1..8538: mol_type = other DNA

SEQ ID NO: 60 - DNA, 8538 bp, synthetic construct; identical to SEQ ID NO: 58
  source 1..8538: mol_type = other DNA

SEQ ID NO: 61 - DNA, 8538 bp, synthetic construct; identical to SEQ ID NO: 58
  source 1..8538: mol_type = other DNA

SEQ ID NO: 62 - protein, 529 aa, Homo sapiens
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 63 - RNA, 1590 nt, Homo sapiens
  source 1..1590: mol_type = other RNA
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacatagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgaattgctgaggcgaggtgtccaggcccaacccctcaatgtaggcttctgtgagcaggagtttgaacagacctga

SEQ ID NO: 64 - RNA, 1590 nt, synthetic construct
  source 1..1590: mol_type = other RNA
atgacttcttcacgtctgtggttcagcctcctgctcgcagcggcgtttgccggtcgggctacggccttgtggccatggccccaaaacttccagacatcagatcagcggtatgtgctgtatccgaacaattttcaattccagtacgatgtgagcagcgcggcacagccgggctgctccgtactggatgaagcgttccagcgctaccgcgatctgttgtttggaagtgggtcatggccacggccgtacctgaccggcaaacgccacaccctggagaagaatgttcttgttgtcagcgtagtcacccccgggtgcaatcagcttcccacccttgagagtgtagaaaactacactctcactattaacgatgaccagtgccttctgctctcggagaccgtgtggggggctctacgcgggctggagacctttagccaactggtgtggaagagcgcggaagggactttcttcatcaataagacggagattgaggactttccgcgctttccacaccgggggctgttgttggacacctcccgccactatctcccgctgtcgagcatactcgacacccttgacgtcatggcgtataataagttgaatgtgtttcactggcatttagttgacgacccctcttttccgtatgagtcctttacttttccggaactcatgcggaaggggtcgtacaacccagtgacgcacatttatactgcgcaggacgtcaaggaggtgatcgagtatgctcgactgcgggggatacgtgtgctcgcggagtttgatactcccgggcacacgctctcatggggtcctggaatccccgggttgctcaccccgtgttattcaggctcggagccatcgggcacttttggcccggtgaatccgagcctgaataacacgtacgagtttatgtccaccttcttcctcgaggtgagtagtgtcttccctgacttctaccttcacctcgggggagatgaggtggactttacttgttggaagagcaacccggagatccaggacttcatgagaaagaagggttttggggaggacttcaagcaattggagtccttctacattcagacccttctcgacatagtctcttcctacgggaagggctatgtcgtgtggcaggaggtgttcgacaacaaggtcaaaatccagcccgatacgatcatccaggtctggcgcgaggatatcccggttaattatatgaaggagctggagctggtcacgaaagcaggctttcgtgccctgctctcagctccttggtatcttaaccggatatcctacgggccagactggaaggatttttatatcgtagagcccctcgcatttgaaggtactccggagcagaaggcactggtcatcgggggggaagcttgcatgtggggggagtacgttgacaacacgaaccttgttcccaggttgtggccccgcgcaggggctgtggctgaacgcctctggagcaacaagctcacatcggatttgacttttgcgtacgagcgcttaagtcatttccgatgtgagctgttgcgcagaggcgttcaggcacagcccctgaacgtggggttctgcgagcaggagtttgagcagacgtga

SEQ ID NO: 65 - RNA, 42 nt, Homo sapiens
  source 1..42: mol_type = other RNA
ctcacgtggccagccccctccgagaggggagaccagcgggcc

SEQ ID NO: 66 - RNA, 3153 nt, Homo sapiens
  source 1..3153: mol_type = other RNA
gccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacagaagagcctctcccccatgcatactcatccacctccctcccctagagctattctcctttgggtttcttgctgcttcaattttatacaaccattatttaaatattattaaacacatattgttctctaggcactgtggtagtgggtttttttgttgtttttgtttttgagactgtctcaaaaactctgtcgcccaggctgacagtgcagtggcacaatcttggctcactgcagcctctgcctcctgggttcaagcgattctcgtgcatcagcctcctgagtaactggaattaataggcacgtgccaccatgtccatctaattcatatatatatattttttttttctgagacggagtctcactgtcacacaggctggagtgcagtggcacgatctcgactcactgcaagctccacctcctgggttcacgccattctcctgcctcagcctccccagtagctgggactacaggcgcccgccaccacgcccggctaattttttgtatttttagtagagatggggtttctccgtgttagccaggatggtctcgatctcctgacctcgtgatccgcccgccttggcctcccaaagtgctgggattacaggcgtgagccaccgcgcccggccgaattcatctatttttagtagagatggggtttcactatattggccaggctggtcttgaactcatgacctcagatgttcacttgtcttggcctcccaaagtgctgggattagaggcgtgagccaccgcacgcgggcctgtggtaaattgttgaatttgaaggactcagaggccctggtcaattccaaaataacgtaggcgacttccatccccctcctcccaaccattttcagcccaaagcatcttcgcagggaatggatggctgcgcggaggtgggcggtggctctggagagggtctttgcaggtgtgattttctctagaaggaaatgtctcgtcgtggacccagactgccccctcctggtttcagatgcagaagtgatactgtaagccagaggcgggggcagtaatgcatcgcagccattttaggtgaggatttccttggcggttatttgttaagttctttggctgggccctgggctggggtaacaatggacaggttccaggcatttttttcagaaagcttccagtgtagtggatacagaaacttcaggaaggcagggctgagaaggatctgagtaaaactcggtccttcaacaccatccttcagcccctgggtcatgttccttcgaggtcctggtgggaggtagacaagcctagccttgtgctgttcctgtaaggacagggtgggcattttctaccaacagaattcttggaattttcacacagcccagcctagccaagtccagggctatagcccagatacacaagttaaggtcccagcactggcacccaccacaggagcccccttacctctattacccagaagcttgtaggaggggtggtccgcagacaaggaccctgcacaggtgcgaccctgcttccctcctggtcataactttcatgttactattgcttgggataatgttaagtaaaaatagcagacactgagttttaagtctcaagtggatgaaggcagagatcgtgatacacttgagttaaagcagtagggttctgtcattttctattcctgttgtaaacattttctttaatgttattatttttaccactaaactaacgtggcctggtcacgactttcattggtaaagtgtgctgttcctcaccctccaccgttgctcctttggtccactgatcataagagcatttacctgaaggtcgtcagacctcgaatgccaacaggtcaactgcagtggcctgcagttaccacccagtctgttccaatgaacagaatcgctgttgccccaactcatctcccttcacctaggctgtaaattgaaagtcccacccctgagcggaacacaggccatcttgtgtgctgtgcaccaccagggggtggggaagtttccagactgacttcctggctccagtcatcctaggaaaagagttctccagtcgctccccacccccaccccttcccattccaggagtctattaaggaggcaaagcaggcctaacgggtatcaaagcaaaggagtgaatggagactgggagagtcttcaacctctcctctccttggtaggagctgaggctgcatgccaggtaccttcccttcgaggaatctaataaagctaggtcactggtgttttcaggtgcttctcaaaggattgccgtaggggtaggatatcaggatgtgggagcacaggtgccaccacagcactagtgatggagagtcattgcccctagacttctgggacagtgagactgtgaggaaagctgaaatgatactgggaaagggtgaaagaaaggatgtaggtggaatttatttagtattaatgtaggtacacataccttatggcaacattcctagcactctaaattctagatttgtatagtttctgtcaatatcttttgtaagcttaatcaatacagggcatgacaagtatgtgtcacatacttttttttccacgaagaaaaaaaataagtaggaattgggtgctttgtttatcaaaatttgtatttcctttataaataaactttgaaataaaggttgaaaattagta

SEQ ID NO: 67 - RNA, 4785 nt, synthetic construct
  source 1..4785: mol_type = other RNA
ctcacgtggccagccccctccgagaggggagaccagcgggccatgacttcttcacgtctgtggttcagcctcctgctcgcagcggcgtttgccggtcgggctacggccttgtggccatggccccaaaacttccagacatcagatcagcggtatgtgctgtatccgaacaattttcaattccagtacgatgtgagcagcgcggcacagccgggctgctccgtactggatgaagcgttccagcgctaccgcgatctgttgtttggaagtgggtcatggccacggccgtacctgaccggcaaacgccacaccctggagaagaatgttcttgttgtcagcgtagtcacccccgggtgcaatcagcttcccacccttgagagtgtagaaaactacactctcactattaacgatgaccagtgccttctgctctcggagaccgtgtggggggctctacgcgggctggagacctttagccaactggtgtggaagagcgcggaagggactttcttcatcaataagacggagattgaggactttccgcgctttccacaccgggggctgttgttggacacctcccgccactatctcccgctgtcgagcatactcgacacccttgacgtcatggcgtataataagttgaatgtgtttcactggcatttagttgacgacccctcttttccgtatgagtcctttacttttccggaactcatgcggaaggggtcgtacaacccagtgacgcacatttatactgcgcaggacgtcaaggaggtgatcgagtatgctcgactgcgggggatacgtgtgctcgcggagtttgatactcccgggcacacgctctcatggggtcctggaatccccgggttgctcaccccgtgttattcaggctcggagccatcgggcacttttggcccggtgaatccgagcctgaataacacgtacgagtttatgtccaccttcttcctcgaggtgagtagtgtcttccctgacttctaccttcacctcgggggagatgaggtggactttacttgttggaagagcaacccggagatccaggacttcatgagaaagaagggttttggggaggacttcaagcaattggagtccttctacattcagacccttctcgacatagtctcttcctacgggaagggctatgtcgtgtggcaggaggtgttcgacaacaaggtcaaaatccagcccgatacgatcatccaggtctggcgcgaggatatcccggttaattatatgaaggagctggagctggtcacgaaagcaggctttcgtgccctgctctcagctccttggtatcttaaccggatatcctacgggccagactggaaggatttttatatcgtagagcccctcgcatttgaaggtactccggagcagaaggcactggtcatcgggggggaagcttgcatgtggggggagtacgttgacaacacgaaccttgttcccaggttgtggccccgcgcaggggctgtggctgaacgcctctggagcaacaagctcacatcggatttgacttttgcgtacgagcgcttaagtcatttccgatgtgagctgttgcgcagaggcgttcaggcacagcccctgaacgtggggttctgcgagcaggagtttgagcagacgtgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacagaagagcctctcccccatgcatactcatccacctccctcccctagagctattctcctttgggtttcttgctgcttcaattttatacaaccattatttaaatattattaaacacatattgttctctaggcactgtggtagtgggtttttttgttgtttttgtttttgagactgtctcaaaaactctgtcgcccaggctgacagtgcagtggcacaatcttggctcactgcagcctctgcctcctgggttcaagcgattctcgtgcatcagcctcctgagtaactggaattaataggcacgtgccaccatgtccatctaattcatatatatatattttttttttctgagacggagtctcactgtcacacaggctggagtgcagtggcacgatctcgactcactgcaagctccacctcctgggttcacgccattctcctgcctcagcctccccagtagctgggactacaggcgcccgccaccacgcccggctaattttttgtatttttagtagagatggggtttctccgtgttagccaggatggtctcgatctcctgacctcgtgatccgcccgccttggcctcccaaagtgctgggattacaggcgtgagccaccgcgcccggccgaattcatctatttttagtagagatggggtttcactatattggccaggctggtcttgaactcatgacctcagatgttcacttgtcttggcctcccaaagtgctgggattagaggcgtgagccaccgcacgcgggcctgtggtaaattgttgaatttgaaggactcagaggccctggtcaattccaaaataacgtaggcgacttccatccccctcctcccaaccattttcagcccaaagcatcttcgcagggaatggatggctgcgcggaggtgggcggtggctctggagagggtctttgcaggtgtgattttctctagaaggaaatgtctcgtcgtggacccagactgccccctcctggtttcagatgcagaagtgatactgtaagccagaggcgggggcagtaatgcatcgcagccattttaggtgaggatttccttggcggttatttgttaagttctttggctgggccctgggctggggtaacaatggacaggttccaggcatttttttcagaaagcttccagtgtagtggatacagaaacttcaggaaggcagggctgagaaggatctgagtaaaactcggtccttcaacaccatccttcagcccctgggtcatgttccttcgaggtcctggtgggaggtagacaagcctagccttgtgctgttcctgtaaggacagggtgggcattttctaccaacagaattcttggaattttcacacagcccagcctagccaagtccagggctatagcccagatacacaagttaaggtcccagcactggcacccaccacaggagcccccttacctctattacccagaagcttgtaggaggggtggtccgcagacaaggaccctgcacaggtgcgaccctgcttccctcctggtcataactttcatgttactattgcttgggataatgttaagtaaaaatagcagacactgagttttaagtctcaagtggatgaaggcagagatcgtgatacacttgagttaaagcagtagggttctgtcattttctattcctgttgtaaacattttctttaatgttattatttttaccactaaactaacgtggcctggtcacgactttcattggtaaagtgtgctgttcctcaccctccaccgttgctcctttggtccactgatcataagagcatttacctgaaggtcgtcagacctcgaatgccaacaggtcaactgcagtggcctgcagttaccacccagtctgttccaatgaacagaatcgctgttgccccaactcatctcccttcacctaggctgtaaattgaaagtcccacccctgagcggaacacaggccatcttgtgtgctgtgcaccaccagggggtggggaagtttccagactgacttcctggctccagtcatcctaggaaaagagttctccagtcgctccccacccccaccccttcccattccaggagtctattaaggaggcaaagcaggcctaacgggtatcaaagcaaaggagtgaatggagactgggagagtcttcaacctctcctctccttggtaggagctgaggctgcatgccaggtaccttcccttcgaggaatctaataaagctaggtcactggtgttttcaggtgcttctcaaaggattgccgtaggggtaggatatcaggatgtgggagcacaggtgccaccacagcactagtgatggagagtcattgcccctagacttctgggacagtgagactgtgaggaaagctgaaatgatactgggaaagggtgaaagaaaggatgtaggtggaatttatttagtattaatgtaggtacacataccttatggcaacattcctagcactctaaattctagatttgtatagtttctgtcaatatcttttgtaagcttaatcaatacagggcatgacaagtatgtgtcacatacttttttttccacgaagaaaaaaaataagtaggaattgggtgctttgtttatcaaaatttgtatttcctttataaataaactttgaaataaaggttgaaaattagta

SEQ ID NO: 68 - RNA, 4785 nt, synthetic construct
  source 1..4785: mol_type = other RNA
ctcacgtggccagccccctccgagaggggagaccagcgggccatgaccagcagcaggctgtggttcagcctgctgctggccgcggccttcgctggtcgggccacggccttatggccgtggccccaaaattttcagacgtcggaccagcgttacgtgttgtacccgaacaacttccagttccaatacgacgtgagctcggcagcgcagcccggctgcagcgttctcgacgaggcgttccagcggtaccgcgacctgttatttgggtcggggtcatggccccgaccctatttgacaggtaagcggcacacgctggaaaaaaacgtcctcgtcgtgagcgttgtaacgccgggctgcaatcagctgccgacgctcgagtcggttgagaactacaccctcaccattaacgatgaccagtgccttctgctctcggagaccgtgtggggggctctacgcgggctggagacctttagccaactggtgtggaagagcgcggaagggactttcttcatcaataagacggagattgaggactttccgcgctttccacaccgggggctgttgttggacacctcccgccactatctcccgctgtcgagcatactcgacacccttgacgtcatggcgtataataagttgaatgtgtttcactggcatttagttgacgacccctcttttccgtatgagtcctttacttttccggaactcatgcggaaggggtcgtacaacccagtgacgcacatttatactgcgcaggacgtcaaggaggtgatcgagtatgctcgactgcgggggatacgtgtgctcgcggagtttgatactcccgggcacacgctctcatggggtcctggaatccccgggttgctcaccccgtgttattcaggctcggagccatcgggcacttttggcccggtgaatccgagcctgaataacacgtacgagtttatgtccaccttcttcctcgaggtgagtagtgtcttccctgacttctaccttcacctcgggggagatgaggtggactttacttgttggaagagcaacccggagatccaggacttcatgagaaagaagggttttggggaggacttcaagcaattggagtccttctacattcagacccttctcgacatagtctcttcctacgggaagggctatgtcgtgtggcaggaggtgttcgacaacaaggtcaaaatccagcccgatacgatcatccaggtctggcgcgaggatatcccggttaattatatgaaggagctggagctggtcacgaaagcaggctttcgtgccctgctctcagctccttggtatcttaaccggatatcctacgggccagactggaaggatttttatatcgtagagcccctcgcatttgaaggtactccggagcagaaggcactggtcatcggtggtgaggcgtgtatgtggggggagtacgtggacaacacgaacctggtcccacgtctctggccacgtgcgggcgcggtggcggagcgcctgtggtctaataagctcacgtctgacttgacttttgcttacgagcgcctgagccacttccggtgcgagctattgcgccggggggttcaggcgcaacccctaaatgtggggttttgtgagcaagagttcgagcagacgtgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacagaagagcctctcccccatgcatactcatccacctccctcccctagagctattctcctttgggtttcttgctgcttcaattttatacaaccattatttaaatattattaaacacatattgttctctaggcactgtggtagtgggtttttttgttgtttttgtttttgagactgtctcaaaaactctgtcgcccaggctgacagtgcagtggcacaatcttggctcactgcagcctctgcctcctgggttcaagcgattctcgtgcatcagcctcctgagtaactggaattaataggcacgtgccaccatgtccatctaattcatatatatatattttttttttctgagacggagtctcactgtcacacaggctggagtgcagtggcacgatctcgactcactgcaagctccacctcctgggttcacgccattctcctgcctcagcctccccagtagctgggactacaggcgcccgccaccacgcccggctaattttttgtatttttagtagagatggggtttctccgtgttagccaggatggtctcgatctcctgacctcgtgatccgcccgccttggcctcccaaagtgctgggattacaggcgtgagccaccgcgcccggccgaattcatctatttttagtagagatggggtttcactatattggccaggctggtcttgaactcatgacctcagatgttcacttgtcttggcctcccaaagtgctgggattagaggcgtgagccaccgcacgcgggcctgtggtaaattgttgaatttgaaggactcagaggccctggtcaattccaaaataacgtaggcgacttccatccccctcctcccaaccattttcagcccaaagcatcttcgcagggaatggatggctgcgcggaggtgggcggtggctctggagagggtctttgcaggtgtgattttctctagaaggaaatgtctcgtcgtggacccagactgccccctcctggtttcagatgcagaagtgatactgtaagccagaggcgggggcagtaatgcatcgcagccattttaggtgaggatttccttggcggttatttgttaagttctttggctgggccctgggctggggtaacaatggacaggttccaggcatttttttcagaaagcttccagtgtagtggatacagaaacttcaggaaggcagggctgagaaggatctgagtaaaactcggtccttcaacaccatccttcagcccctgggtcatgttccttcgaggtcctggtgggaggtagacaagcctagccttgtgctgttcctgtaaggacagggtgggcattttctaccaacagaattcttggaattttcacacagcccagcctagccaagtccagggctatagcccagatacacaagttaaggtcccagcactggcacccaccacaggagcccccttacctctattacccagaagcttgtaggaggggtggtccgcagacaaggaccctgcacaggtgcgaccctgcttccctcctggtcataactttcatgttactattgcttgggataatgttaagtaaaaatagcagacactgagttttaagtctcaagtggatgaaggcagagatcgtgatacacttgagttaaagcagtagggttctgtcattttctattcctgttgtaaacattttctttaatgttattatttttaccactaaactaacgtggcctggtcacgactttcattggtaaagtgtgctgttcctcaccctccaccgttgctcctttggtccactgatcataagagcatttacctgaaggtcgtcagacctcgaatgccaacaggtcaactgcagtggcctgcagttaccacccagtctgttccaatgaacagaatcgctgttgccccaactcatctcccttcacctaggctgtaaattgaaagtcccacccctgagcggaacacaggccatcttgtgtgctgtgcaccaccagggggtggggaagtttccagactgacttcctggctccagtcatcctaggaaaagagttctccagtcgctccccacccccaccccttcccattccaggagtctattaaggaggcaaagcaggcctaacgggtatcaaagcaaaggagtgaatggagactgggagagtcttcaacctctcctctccttggtaggagctgaggctgcatgccaggtaccttcccttcgaggaatctaataaagctaggtcactggtgttttcaggtgcttctcaaaggattgccgtaggggtaggatatcaggatgtgggagcacaggtgccaccacagcactagtgatggagagtcattgcccctagacttctgggacagtgagactgtgaggaaagctgaaatgatactgggaaagggtgaaagaaaggatgtaggtggaatttatttagtattaatgtaggtacacataccttatggcaacattcctagcactctaaattctagatttgtatagtttctgtcaatatcttttgtaagcttaatcaatacagggcatgacaagtatgtgtcacatacttttttttccacgaagaaaaaaaataagtaggaattgggtgctttgtttatcaaaatttgtatttcctttataaataaactttgaaataaaggttgaaaattagta

SEQ ID NO: 69 - RNA, 4785 nt, synthetic construct
  source 1..4785: mol_type = other RNA
ctcacgtggccagccccctccgagaggggagaccagcgggccatgacttcttcaaggctttggtttagcctcctgctcgcagcggcgtttgccggacgggctacggccctctggccctggcctcaaaacttccagacatccgaccagcgctatgtcctttacccgaacaattttcaattccagtacgatgtcagctcggccgcacagccgggctgctcagtcctcgatgaagccttccagcgctatcgtgatctgctttttggttccgggtcttggccccgtccgtacctgaccgggaaacggcatacactggagaagaatgtgttggttgtctctgtagtcacacctggatgtaaccagcttcctaccttggagtcagtggaaaactacactctgaccataaatgatgaccagtgtttactcctctcggagaccgtgtggggggctctacgcggtctggagacctttagccaactggtttggaagtctgcggaggggactttcttcatcaataagactgagattgaggactttccccgctttcctcaccggggcttgctgttggacacatctcgccactatctgccgctgtcgagcatactcgacacccttgatgtcatggcgtacaataaattgaacgtgtttcactggcatttagttgacgacccctcttttccgtatgagtcctttacttttccggaactcatgcggaaggggtcgtacaaccctgtcacgcacatttatactgcgcaggacgtcaaggaggtgatcgagtatgctcgactgcgggggatccgtgtgctcgcggagtttgatactcctggccacactctctcatggggtcctggaatccccgggttgctcaccccgtgttattcaggctcggagccatcgggcacttttggcccggtgaatccgagcctgaataacacgtacgagtttatgtccaccttcttcctcgaggtgagtagtgtcttccctgacttctaccttcacctcgggggagatgaggtggactttacttgttggaagagcaacccggagatccaggacttcatgagaaagaagggttttggggaggacttcaagcaattggagtccttctacattcagacccttctcgacatagtctcttcctacgggaagggctatgtcgtgtggcaggaggtgtttgacaacaaggtcaaaattcagcccgatacgatcatacaggtctggcgagaggatattccagtgaattatatgaaggagctggagctggtcacgaaagccggctttcgggcccttctctctgccccctggtacctgaaccgtatatcctatggccctgactggaaggatttctatatagtagaacccctggcatttgaaggtactcctgagcagaaggctctggtgattggtggagaggcttgcatgtggggggaatacgttgacaacacgaaccttgttcccaggttgtggccccgcgcaggggctgtggctgaacgcctctggagcaacaagctcacatcggatttgacttttgcgtacgagcgcttaagtcatttccgatgtgagctgttgcgcagaggcgttcaggcacagcccctgaacgtaggcttctgtgagcaggagtttgaacagacctgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacagaagagcctctcccccatgcatactcatccacctccctcccctagagctattctcctttgggtttcttgctgcttcaattttatacaaccattatttaaatattattaaacacatattgttctctaggcactgtggtagtgggtttttttgttgtttttgtttttgagactgtctcaaaaactctgtcgcccaggctgacagtgcagtggcacaatcttggctcactgcagcctctgcctcctgggttcaagcgattctcgtgcatcagcctcctgagtaactggaattaataggcacgtgccaccatgtccatctaattcatatatatatattttttttttctgagacggagtctcactgtcacacaggctggagtgcagtggcacgatctcgactcactgcaagctccacctcctgggttcacgccattctcctgcctcagcctccccagtagctgggactacaggcgcccgccaccacgcccggctaattttttgtatttttagtagagatggggtttctccgtgttagccaggatggtctcgatctcctgacctcgtgatccgcccgccttggcctcccaaagtgctgggattacaggcgtgagccaccgcgcccggccgaattcatctatttttagtagagatggggtttcactatattggccaggctggtcttgaactcatgacctcagatgttcacttgtcttggcctcccaaagtgctgggattagaggcgtgagccaccgcacgcgggcctgtggtaaattgttgaatttgaaggactcagaggccctggtcaattccaaaataacgtaggcgacttccatccccctcctcccaaccattttcagcccaaagcatcttcgcagggaatggatggctgcgcggaggtgggcggtggctctggagagggtctttgcaggtgtgattttctctagaaggaaatgtctcgtcgtggacccagactgccccctcctggtttcagatgcagaagtgatactgtaagccagaggcgggggcagtaatgcatcgcagccattttaggtgaggatttccttggcggttatttgttaagttctttggctgggccctgggctggggtaacaatggacaggttccaggcatttttttcagaaagcttccagtgtagtggatacagaaacttcaggaaggcagggctgagaaggatctgagtaaaactcggtccttcaacaccatccttcagcccctgggtcatgttccttcgaggtcctggtgggaggtagacaagcctagccttgtgctgttcctgtaaggacagggtgggcattttctaccaacagaattcttggaattttcacacagcccagcctagccaagtccagggctatagcccagatacacaagttaaggtcccagcactggcacccaccacaggagcccccttacctctattacccagaagcttgtaggaggggtggtccgcagacaaggaccctgcacaggtgcgaccctgcttccctcctggtcataactttcatgttactattgcttgggataatgttaagtaaaaatagcagacactgagttttaagtctcaagtggatgaaggcagagatcgtgatacacttgagttaaagcagtagggttctgtcattttctattcctgttgtaaacattttctttaatgttattatttttaccactaaactaacgtggcctggtcacgactttcattggtaaagtgtgctgttcctcaccctccaccgttgctcctttggtccactgatcataagagcatttacctgaaggtcgtcagacctcgaatgccaacaggtcaactgcagtggcctgcagttaccacccagtctgttccaatgaacagaatcgctgttgccccaactcatctcccttcacctaggctgtaaattgaaagtcccacccctgagcggaacacaggccatcttgtgtgctgtgcaccaccagggggtggggaagtttccagactgacttcctggctccagtcatcctaggaaaagagttctccagtcgctccccacccccaccccttcccattccaggagtctattaaggaggcaaagcaggcctaacgggtatcaaagcaaaggagtgaatggagactgggagagtcttcaacctctcctctccttggtaggagctgaggctgcatgccaggtaccttcccttcgaggaatctaataaagctaggtcactggtgttttcaggtgcttctcaaaggattgccgtaggggtaggatatcaggatgtgggagcacaggtgccaccacagcactagtgatggagagtcattgcccctagacttctgggacagtgagactgtgaggaaagctgaaatgatactgggaaagggtgaaagaaaggatgtaggtggaatttatttagtattaatgtaggtacacataccttatggcaacattcctagcactctaaattctagatttgtatagtttctgtcaatatcttttgtaagcttaatcaatacagggcatgacaagtatgtgtcacatacttttttttccacgaagaaaaaaaataagtaggaattgggtgctttgtttatcaaaatttgtatttcctttataaataaactttgaaataaaggttgaaaattagta

SEQ ID NO: 70 - RNA, 444 nt, Homo sapiens
  source 1..444: mol_type = other RNA
atggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccatgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 71 - RNA, 444 nt, Homo sapiens
  source 1..444: mol_type = other RNA
tcattccttgggattggtgacgacagccgtggtggaataggagtaggggctcagcagggcggcaatggtgtagcggcgggggccggagtcgttggctgtgaataccacctctgcatgctcatggaatggggagatgccaagtgccttccagtaagatttggtgtctatttccactttgtatatcccttctacaaattcctcctcagttgtgagcccatgcagctctccagactcactggttttcccagaggcaaatggctcccaggtgtcatcagcagcctttctgaacacatgcatggccacattgatggcaggactgcctcggacagcatctagaactttgaccatcagaggacacttggattcaccggtgcccgtagggccagcctcagacacaaataccagtccagcaaggcagaggaggagcagacgatgagaagccat

SEQ ID NO: 72 - DNA, 19 bp, Homo sapiens
  source 1..19: mol_type = other DNA
cctgccatcaatgtggcca

SEQ ID NO: 73 - DNA, 19 bp, Homo sapiens
  source 1..19: mol_type = other DNA
ctgccatcaatgtggccat

SEQ ID NO: 74 - DNA, 17 bp, Homo sapiens
  source 1..17: mol_type = other DNA
ccatcaatgtggccatg

SEQ ID NO: 75 - DNA, 18 bp, Homo sapiens
  source 1..18: mol_type = other DNA
catcaatgtggccatgca

SEQ ID NO: 76 - DNA, 18 bp, Homo sapiens
  source 1..18: mol_type = other DNA
atcaatgtggccatgcat

SEQ ID NO: 77 - DNA, 18 bp, Homo sapiens
  source 1..18: mol_type = other DNA
caatgtggccatgcatgt

SEQ ID NO: 78 - DNA, 18 bp, Homo sapiens
  source 1..18: mol_type = other DNA
atgtggccatgcatgtgt

SEQ ID NO: 79 - DNA, 18 bp, Homo sapiens
  source 1..18: mol_type = other DNA
tgtggccatgcatgtgtt

SEQ ID NO: 80 - DNA, 19 bp, Homo sapiens
  source 1..19: mol_type = other DNA
gtggccatgcatgtgttca

SEQ ID NO: 81 - DNA, 19 bp, Homo sapiens
  source 1..19: mol_type = other DNA
ggccatgcatgtgttcaga

SEQ ID NO: 82 - DNA, 19 bp, Homo sapiens
  source 1..19: mol_type = other DNA
gccatgcatgtgttcagaa

SEQ ID NO: 83 - DNA, 19 bp, Homo sapiens
  source 1..19: mol_type = other DNA
atgcatgtgttcagaaagg

SEQ ID NO: 84 - RNA, 19 nt, synthetic construct
  source 1..19: mol_type = other RNA
tggccacattgatggcagg

SEQ ID NO: 85 - RNA, 19 nt, synthetic construct
  source 1..19: mol_type = other RNA
atggccacattgatggcag

SEQ ID NO: 86 - RNA, 17 nt, synthetic construct
  source 1..17: mol_type = other RNA
catggccacattgatgg

SEQ ID NO: 87 - RNA, 18 nt, synthetic construct
  source 1..18: mol_type = other RNA
tgcatggccacattgatg

SEQ ID NO: 88 - RNA, 18 nt, synthetic construct
  source 1..18: mol_type = other RNA
atgcatggccacattgat

SEQ ID NO: 89 - RNA, 18 nt, synthetic construct
  source 1..18: mol_type = other RNA
acatgcatggccacattg

SEQ ID NO: 90 - RNA, 18 nt, synthetic construct
  source 1..18: mol_type = other RNA
acacatgcatggccacat

SEQ ID NO: 91 - RNA, 18 nt, synthetic construct
  source 1..18: mol_type = other RNA
aacacatgcatggccaca

SEQ ID NO: 92 - RNA, 19 nt, synthetic construct
  source 1..19: mol_type = other RNA
tgaacacatgcatggccac

SEQ ID NO: 93 - RNA, 19 nt, synthetic construct
  source 1..19: mol_type = other RNA
tctgaacacatgcatggcc

SEQ ID NO: 94 - RNA, 19 nt, synthetic construct
  source 1..19: mol_type = other RNA
ttctgaacacatgcatggc

SEQ ID NO: 95 - RNA, 19 nt, synthetic construct
  source 1..19: mol_type = other RNA
cctttctgaacacatgcat

SEQ ID NO: 96 - DNA, 18 bp, Homo sapiens
  source 1..18: mol_type = other DNA
ctgccatcaatgtggcca

SEQ ID NO: 97 - DNA, 16 bp, Homo sapiens
  source 1..16: mol_type = other DNA
ccatcaatgtggccat

SEQ ID NO: 98 - DNA, 16 bp, Homo sapiens
  source 1..16: mol_type = other DNA
tcaatgtggccatgca

SEQ ID NO: 99 - DNA, 17 bp, Homo sapiens
  source 1..17: mol_type = other DNA
caatgtggccatgcatg

SEQ ID NO: 100 - DNA, 16 bp, Homo sapiens
  source 1..16: mol_type = other DNA
gtggccatgcatgtgt

SEQ ID NO: 101 - DNA, 16 bp, Homo sapiens
  source 1..16: mol_type = other DNA
gccatgcatgtgttca

SEQ ID NO: 102 - DNA, 17 bp, Homo sapiens
  source 1..17: mol_type = other DNA
ccatgcatgtgttcaga

SEQ ID NO: 103 - DNA, 17 bp, Homo sapiens
  source 1..17: mol_type = other DNA
catgcatgtgttcagaa

SEQ ID NO: 104 - DNA, 19 bp, Homo sapiens
  source 1..19: mol_type = other DNA
atgcatgtgttcagaaagg

SEQ ID NO: 105 - DNA, 18 bp, Homo sapiens
  source 1..18: mol_type = other DNA
tggccacattgatggcag

SEQ ID NO: 106 - DNA, 16 bp, Homo sapiens
  source 1..16: mol_type = other DNA
atggccacattgatgg

SEQ ID NO: 107 - DNA, 16 bp, Homo sapiens
  source 1..16: mol_type = other DNA
tgcatggccacattga

SEQ ID NO: 108 - DNA, 17 bp, Homo sapiens
  source 1..17: mol_type = other DNA
catgcatggccacattg

SEQ ID NO: 109 - DNA, 16 bp, Homo sapiens
  source 1..16: mol_type = other DNA
acacatgcatggccac

SEQ ID NO: 110 - DNA, 16 bp, Homo sapiens
  source 1..16: mol_type = other DNA
tgaacacatgcatggc

SEQ ID NO: 111 - DNA, 17 bp, Homo sapiens
  source 1..17: mol_type = other DNA
tctgaacacatgcatgg

SEQ ID NO: 112 - DNA, 17 bp, Homo sapiens
  source 1..17: mol_type = other DNA
ttctgaacacatgcatg

SEQ ID NO: 113 - DNA, 19 bp, Homo sapiens
  source 1..19: mol_type = other DNA
cctttctgaacacatgcat

SEQ ID NO: 114 - RNA, 444 nt, Homo sapiens
  source 1..444: mol_type = other RNA
atggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 115 - DNA, 15 bp, Homo sapiens
  source 1..15: mol_type = other DNA
ccatcaatgtggccg

SEQ ID NO: 116 - DNA, 15 bp, Homo sapiens
  source 1..15: mol_type = other DNA
catcaatgtggccgt

SEQ ID NO: 117 - DNA, 15 bp, Homo sapiens
  source 1..15: mol_type = other DNA
tcaatgtggccgtgc

SEQ ID NO: 118 - DNA, 17 bp, Homo sapiens
  source 1..17: mol_type = other DNA
caatgtggccgtgcatg

SEQ ID NO: 119 - DNA, 16 bp, Homo sapiens
  source 1..16: mol_type = other DNA
atgtggccgtgcatgt

SEQ ID NO: 120 - DNA, 16 bp, Homo sapiens
  source 1..16: mol_type = other DNA
tggccgtgcatgtgtt

SEQ ID NO: 121 - DNA, 16 bp, Homo sapiens
  source 1..16: mol_type = other DNA
cgtgcatgtgttcaga

SEQ ID NO: 122 - DNA, 18 bp, Homo sapiens
  source 1..18: mol_type = other DNA
gtgcatgtgttcagaaag

SEQ ID NO: 123 - DNA, 15 bp, Homo sapiens
  source 1..15: mol_type = other DNA
cggccacattgatgg

SEQ ID NO: 124 - DNA, 15 bp, Homo sapiens
  source 1..15: mol_type = other DNA
acggccacattgatg

SEQ ID NO: 125 - DNA, 15 bp, Homo sapiens
  source 1..15: mol_type = other DNA
gcacggccacattga

SEQ ID NO: 126 - DNA, 17 bp, Homo sapiens
  source 1..17: mol_type = other DNA
catgcacggccacattg

SEQ ID NO: 127 - DNA, 16 bp, Homo sapiens
  source 1..16: mol_type = other DNA
acatgcacggccacat

SEQ ID NO: 128 - DNA, 16 bp, Homo sapiens
  source 1..16: mol_type = other DNA
aacacatgcacggcca

SEQ ID NO: 129 - DNA, 16 bp, Homo sapiens
  source 1..16: mol_type = other DNA
tctgaacacatgcacg

SEQ ID NO: 130 - DNA, 18 bp, Homo sapiens
  source 1..18: mol_type = other DNA
ctttctgaacacatgcac

SEQ ID NO: 131 - DNA, 18 bp, Homo sapiens
  source 1..18: mol_type = other DNA
tgcatgtgttcagaaagg

SEQ ID NO: 132 - DNA, 18 bp, Homo sapiens
  source 1..18: mol_type = other DNA
cctttctgaacacatgca

SEQ ID NO: 133 - RNA, 616 nt, synthetic construct
  source 1..616: mol_type = other RNA
acagaagtccactcattcttggcaggatggcctcccatcgcttgctcctcctgtgcctggcgggcctggtcttcgtatcggaggccgggcccacaggcacaggagagagcaagtgtcccctgatggtgaaggtgctcgacgcggttcgcggcagccctgcgataaacgtggcagtccacgtttttcgcaaggctgccgacgatacgtgggagcccttcgcctcagggaaaacctccgaaagtggtgagctccacgggctcaccactgaggaggagttcgtggaagggatctacaaagtagaaattgataccaagagctattggaaagctcttggtatcagtcccttccacgaacatgcggaggtcgttttcactgctaatgattcgggaccacggcggtatactatagctgcgctgctatcgccgtacagctatagtactaccgccgtggtcacgaatcctaaggagtgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggca

SEQ ID NO: 134 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 135 - RNA, 616 nt, synthetic construct
  source 1..616: mol_type = other RNA
acagaagtccactcattcttggcaggatggcctcccatcgcttgctcctcctgtgccttgcgggcctggtcttcgtatcggaggccgggcccacaggcacaggagagagcaagtgtcccctgatggtgaaggtgctcgacgcggtccgaggcagtcctgccataaacgtggccgtccatgtgttcagaaaggctgctgatgatacgtgggagccattcgcctctgggaaaacctccgaaagtggtgagctccacgggctcaccactgaggaggagttcgtagaagggatctacaaagtggaaattgacaccaaaagctattggaaagctcttggcatcagtccattccacgaacatgcggaggtcgttttcactgctaatgattcgggaccacggcggtatactatagctgcgctgctatcgccgtacagctatagtactaccgccgtggtcacgaatcctaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggca

SEQ ID NO: 136 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 137 - RNA, 628 nt, Homo sapiens
  source 1..628: mol_type = mRNA
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 138 - RNA, 628 nt, synthetic construct
  source 1..628: mol_type = other RNA
acatttgcttctgacacaactgtgttcactagcaacctcaaacagacaccatggttcaccttaccccagaggagaagtcagcagtgactgccctctggggtaaggtgaacgtggacgaggtcgggggggaggccctcgggcgccttcttgtcgtgtatccttggactcagagattctttgagtcctttggggatttgtccacaccggacgcggtgatgggcaaccccaaagtcaaggcgcacggcaagaaggtgctcggggccttctccgacggcctcgcccaccttgataacttgaagggcacgttcgccaccctcagcgagctccattgtgataagctgcacgtggacccggagaattttagactccttggcaatgttttggtgtgcgtactcgcacaccattttggcaaggagtttactccgccggtccaggcagcttatcagaaggtggtcgctggggtggcgaacgctcttgcccacaagtatcattaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 139 - RNA, 628 nt, synthetic construct
  source 1..628: mol_type = other RNA
acatttgcttctgacacaactgtgttcactagcaacctcaaacagacaccatggtgcatcttactccagaggagaagtctgcagttactgccctgtggggcaaggtgaacgtggacgaagttggtggtgaggccctcgggaggctgctggtggtgtatccttggactcagagattctttgagtcctttggggatttgtccacaccggacgcggtgatgggcaaccccaaagtcaaggcgcacggcaagaaggtgctcggtgccttctccgacggcctggcccaccttgataacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 140 - RNA, 2525 nt, Homo sapiens
  source 1..2525: mol_type = mRNA
attcgtgaggggtttgtaatgcagggctgaggagtgtccgaagagaatgggcaggtgagcggtgagacagttgttcttccagaagctttgcagtgaaaggaatcaaagaaatggagccgtgtatcagcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 141 - RNA, 1182 nt, Homo sapiens
  source 1..1182: mol_type = other RNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcacttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 142 - RNA, 18 nt, synthetic construct
  source 1..18: mol_type = other RNA
ctcgaagtgctcacgccc

SEQ ID NO: 143 - RNA, 155 nt, Homo sapiens
  source 1..155: mol_type = other RNA
attcgtgaggggtttgtaatgcagggctgaggagtgtccgaagagaatgggcaggtgagcggtgagacagttgttcttccagaagctttgcagtgaaaggaatcaaagaaatggagccgtgtatcagcagccagactgccttccgggtcactgcc

SEQ ID NO: 144 - RNA, 1188 nt, Homo sapiens
  source 1..1188: mol_type = other RNA
cattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 145 - RNA, 2525 nt, synthetic construct
  source 1..2525: mol_type = other RNA
attcgtgaggggtttgtaatgcagggctgaggagtgtccgaagagaatgggcaggtgagcggtgagacagttgttcttccagaagctttgcagtgaaaggaatcaaagaaatggagccgtgtatcagcagccagactgccttccgggtcactgccatggaggagccgcagtcagatcctagcgtcgagccccctctgagccaggaaaccttcagtgacctctggaagttgcttcctgagaacaacgttctgtcccccttgccgtcccaagcaatggatgatttgatgctgtccccggacgacatcgaacaatggttcactgaagatccaggtccagatgaggctcccagaatgccggaggcggctcccccggtggcgcctgcgccggcagcgccgacaccggcggcgccggcgccggcgccatcctggccgctctcttcgtcggtcccttcccagaagacctaccagggtagttatggttttcgtctggggttcttgcattctgggacagccaagtctgtgacatgcacgtattcgccagcgctgaacaaaatgttctgccagctggcgaaaacgtgcccagtgcaattgtgggtcgattcgaccccacctccggggactcgtgtgcgggccatggccatctacaagcagtcccaacacatgactgaggtggtgcggcggtgcccccatcacgagcggtgctccgactcggatgggttggcaccgccgcagcacctcattcgtgtggagggaaatctgcgtgtagaatatttggatgaccggaatactttccggcattcggtggtggtgccatatgagccgcctgaggttgggtcggattgtaccacaattcactataattatatgtgtaactctagctgtatgggcggcatgaaccgccggccgattctcaccataatcacactggaagactctagtggtaatctattgggaaggaacagctttgaggtgcgtgtttgtgcctgccctgggagagacaggcggaccgaggaggagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 146 - RNA, 2525 nt, synthetic construct
  source 1..2525: mol_type = other RNA
attcgtgaggggtttgtaatgcagggctgaggagtgtccgaagagaatgggcaggtgagcggtgagacagttgttcttccagaagctttgcagtgaaaggaatcaaagaaatggagccgtgtatcagcagccagactgccttccgggtcactgccatggaggagccgcagtcagatcctagcgtcgagccccctctgagccaggaaaccttcagtgacctctggaagttgcttcctgagaacaacgttctgtcccccttgccgtcccaagcaatggatgatttgatgctgtccccggacgacatcgaacaatggttcactgaagatccaggtccagatgaggctcccagaatgccggaggcggctcccccggtggcgcctgcgccggcagcgccgacaccggcggcgccggcgccggcgccatcctggccgctctcttcgtcggtcccttcccagaagacctaccagggtagttatggttttcgtctggggttcttgcattctgggacagccaagtctgtgacatgcacgtattcgccagcgctgaacaaaatgttctgccagctggcgaaaacgtgcccagtgcaattgtgggtcgattcgaccccacctccggggactcgtgtgcgggccatggccatctacaagcagtcccaacacatgactgaggtggtgcggcggtgcccccatcacgagcggtgctccgactcggatgggttggcaccgccgcagcacctcattcgtgtggagggaaatctgcgtgtagaatatttggatgaccggaatactttccggcattcggtggtggtgccatatgagccgcctgaggttgggtcggattgtaccacaattcactataattatatgtgtaactctagctgtatgggcggcatgaaccgccggccgattctcaccataatcacactggaagactctagtggtaatctattgggaaggaacagctttgaggtgcgtgtttgtgcctgccctgggagagacaggcggaccgaggaggagaatctccgcaagaaaggggagcctcaccacgagctgcccccagggagcactaagcgagcactgcccaacaacaccagctcctctccccagccaaagaagaaaccactggatggagaatatttcacccttcagatccgtggccgcgaacgcttcgaaatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgcca

SEQ ID NO: 147 - RNA, 2907 nt, Homo sapiens
  source 1..2907: mol_type = mRNA
atggtgaactccagtcgcgtgcagcctcagcagcccggggaygccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatga

SEQ ID NO: 148 - RNA, 18 nt, synthetic construct
  source 1..18: mol_type = other RNA
ttggcatccccgggctgc